(19) United States
(12) Patent Application Publication
(10) Pub. No.: US 2017/0007679 A1
(43) Pub. Date: Jan. 12, 2017

(54) CRISPR/CAS-RELATED METHODS AND COMPOSITIONS FOR TREATING HIV INFECTION AND AIDS

(71) Applicant: EDITAS MEDICINE INC., Cambridge, MA (US)

(72) Inventors: Morgan L. Maeder, Jamaica Plain, MA (US); Ari E. Friedland, Boston, MA (US); G. Grant Welstead, Cambridge, MA (US); David A. Bumcrot, Belmont, MA (US)

(73) Assignee: EDITAS MEDICINE INC., Cambridge, MA (US)

(21) Appl. No.: 15/274,728

(22) Filed: Sep. 23, 2016

Related U.S. Application Data
(63) Continuation of application No. PCT/US2015/022497, filed on Mar. 25, 2015.
(60) Provisional application No. 61/970,237, filed on Mar. 25, 2014.

(51) Int. Cl. A61K 38/46; C12N 15/113; C12N 9/22

(52) U.S. Cl. CPC A61K 38/465; C12N 9/22; C12Y 301/00; C12N 15/1138; A61K 48/00

(57) ABSTRACT

CRISPR/CAS-related compositions and methods for treatment of a subject at risk for or having a HIV infection or AIDS are disclosed.

### REFERENCE TO RELATED APPLICATIONS

This application is a continuation of PCT International Patent Application No. PCT/US2015/022497, filed on Mar. 25, 2015, which claims the benefit of U.S. Provisional Application No. 61/970,237, filed Mar. 25, 2014, the contents of each of which are hereby incorporated by reference in their entirety herein, and to each of which priority is claimed.

### SEQUENCE LISTING

The specification further incorporates by reference the Sequence Listing submitted herewith via EFS on Sep. 23, 2016. Pursuant to 37 C.F.R. §1.52(e)(5), the Sequence Listing text file, identified as 084177.0124SEQ.txt, is 2,093,238 bytes and was created on Sep. 23, 2016. The Sequence Listing, electronically filed herewith, does not extend beyond the scope of the specification and thus does not contain new matter.

### FIELD OF THE INVENTION

The invention relates to CRISPR/CAS-related methods and components for editing of a target nucleic acid sequence, and applications thereof in connection with Human Immunodeficiency Virus (HIV) infection and Acquired Immunodeficiency Syndrome (AIDS).

### BACKGROUND

Human Immunodeficiency Virus (HIV) is a virus that causes severe immunodeficiency. In the United States, more than 1 million people are infected with the virus. Worldwide, approximately 30-40 million people are infected.

HIV preferentially infects CD4 T cells. It causes declining CD4 T cell counts, severe opportunistic infections and certain cancers, including Kaposi's sarcoma and Burkitt's lymphoma. Untreated HIV infection is a chronic, progressive disease that leads to acquired immunodeficiency syndrome (AIDS) and death in nearly all subjects.

HIV was untreatable and invariably led to death in all subjects until the late 1980's. Since then, antiretroviral therapy (ART) has dramatically slowed the course of HIV infection. Highly active antiretroviral therapy (HAART) is the use of three or more agents in combination to slow HIV. Treatment with HAART has significantly altered the life expectancy of those infected with HIV. A subject in the developed world who maintains their HAART regimen can expect to live into his or her 60's and possibly 70's. However, HAART regimens are associated with significant, long-term side effects. The dosing regimens are complex and associated with strict dietary requirements. Compliance rates with dosing can be lower than 50% in some populations in the United States. In addition, there are significant toxicities associated with HAART treatment, including diabetes, nausea, malaise and sleep disturbances. A subject who does not adhere to dosing requirements of HAART therapy may have a return of viral load in their blood and is at risk for progression of the disease and its associated complications.

HIV is a single-stranded RNA virus that preferentially infects CD4 T-cells. The virus must bind to receptors and coreceptors on the surface of CD4 cells to enter and infect these cells. This binding and infection step is vital to the pathogenesis of HIV. The virus attaches to the CD4 receptor on the cell surface via its own surface glycoproteins, gp120 and gp41. Gp120 binds to a CD4 receptor and must also bind to another coreceptor in order for the virus to enter the host cell. In macrophage-(M-tropic) viruses, the coreceptor is CCR5, also referred to as the CCR5 receptor. CCR5 receptors are expressed by CD4 cells, T cells, gut-associated lymphoid tissue (GALT), macrophages, dendritic cells and microglia. HIV establishes initial infection and replicates in the host most commonly via CCR5 co-receptors.

As most HIV infections and early stage HIV is due to entry and propagation of M-tropic virus, CCR5-Δ32 mutation results in a non-functional CCR5 receptor that does not allow M-tropic HIV-1 virus entry. Individuals carrying two copies of the CCR5-Δ32 allele are resistant to HIV infection and CCR5-Δ32 heterozygous carriers have slow progression of the disease.

CCR5 antagonists (e.g. maraviroc) exist and are used in the treatment of HIV. However, current CCR5 antagonists decrease HIV progression but cannot cure the disease. In addition, there are considerable risks of side effects of these CCR5 antagonists, including severe liver toxicity.

In spite of considerable advances in the treatment of HIV, there remain considerable needs for agents that could prevent, treat, and eliminate HIV infection or AIDS. Therapies that are free from significant toxicities and involve a single or multi-dose regimen (versus current daily dose regimen for the lifetime of a patient) would be superior to current HIV treatment. A reduction or complete elimination of CCR5 expression in myeloid and lymphoid cells would prevent HIV infection and progression, and even cure this disease.

### SUMMARY OF THE INVENTION

Methods and compositions discussed herein, allow for the prevention and treatment of HIV infection and AIDS, by introducing one or more mutations in the gene for C-C chemokine receptor type 5 (CCR5). The CCR5 gene is also known as CKR5, CCR-5, CD195, CKR-5, CCCKR5, CMKBR5, IDDM22, and CC-CKR-5.

Methods and compositions discussed herein, provide for prevention or reduction of HIV infection and/or prevention or reduction of the ability for HIV to enter host cells, e.g., in subjects who are already infected. Exemplary host cells for HIV include, but are not limited to, CD4 cells, T cells, gut associated lymphatic tissue (GALT), macrophages, dendritic cells, myeloid precursor cell, and microglia. Viral entry into the host cells requires interaction of the viral glycoproteins gp41 and gp120 with both the CD4 receptor and a co-receptor, e.g., CCR5. If a co-receptor, e.g., CCR5, is not present on the surface of the host cells, the virus cannot bind and enter the host cells. The progress of the disease is thus impeded. By knocking out or knocking down CCR5 in the host cells, e.g., by introducing a protective mutation (such as a CCR5 delta 32 mutation), entry of the HIV virus into the host cells is prevented.

Methods and compositions discussed herein, provide for treating or delaying the onset or progression of HIV infection or AIDS by gene editing, e.g., using CRISPR-Cas9 mediated methods to alter a CCR5 gene. Altering the CCR5 gene herein refers to reducing or eliminating (1) CCR5 gene expression, (2) CCR5 protein function, or (3) the level of CCR5 protein.

In one aspect, the methods and compositions discussed herein, inhibit or block a critical aspect of the HIV life cycle, i.e., CCR5-mediated entry into T cells, by alteration (e.g., inactivation) of the CCR5 gene. Exemplary mechanisms that can be associated with the alteration of the CCR5 gene include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion. Alteration of the CCR5 gene, e.g., mediated by NHEJ, can result in a mutation, which typically comprises a deletion or insertion (indel). The introduced mutation can take place in any region of the CCR5 gene, e.g., a promoter region or other non-coding region, or a coding region, so long as the mutation results in reduced or loss of the ability to mediate HIV entry into the cell.

In another aspect, the methods and compositions discussed herein may be used to alter the CCR5 gene to treat or prevent HIV infection or AIDS by targeting the coding sequence of the CCR5 gene.

In an embodiment, the gene, e.g., the coding sequence of the CCR5 gene, is targeted to knock out the gene, e.g., to eliminate expression of the gene, e.g., to knock out both alleles of the CCR5 gene, e.g., by introduction of an alteration comprising a mutation (e.g., an insertion or deletion) in the CCR5 gene. This type of alteration is sometimes referred to as “knocking out” the CCR5 gene. While not wishing to be bound by theory, in an embodiment, a targeted knockout approach is mediated by NHEJ using a CRISPR/Cas system comprising a Cas9 molecule, e.g., an enzymatically active Cas9 (eaCas9) molecule, as described herein.

In another aspect, the methods and compositions discussed herein may be used to alter the CCR5 gene to treat or prevent HIV infection or AIDS by targeting a non-coding sequence of the CCR5 gene, e.g., a promoter, an enhancer, an intron, a 3′UTR, and/or a polyadenylation signal.

In one embodiment, the gene, e.g., the non-coding sequence of the CCR5 gene, is targeted to knock out the gene, e.g., to eliminate expression of the gene, e.g., to knock out both alleles of the CCR5 gene, e.g., by introduction of an alteration comprising a mutation (e.g., an insertion or deletion) in the CCR5 gene. In an embodiment, the method provides an alteration that comprises an insertion or deletion. This type of alteration is also sometimes referred to as “knocking out” the CCR5 gene. While not wishing to be bound by theory, in an embodiment, a targeted knockout approach is mediated by NHEJ using a CRISPR/Cas system comprising a Cas9 molecule, e.g., an enzymatically active Cas9 (eaCas9) molecule, as described herein.

In an embodiment, methods and compositions discussed herein, provide for altering (e.g., knocking out) the CCR5 gene. In an embodiment, knocking out the CCR5 gene herein refers to (1) insertion or deletion (e.g., NHEJ-mediated insertion or deletion) of one or more nucleotides of the CCR5 gene (e.g., in close proximity to or within an early coding region or in a non-coding region), or (2) deletion (e.g., NHEJ-mediated deletion) of a genomic sequence of the CCR5 gene (e.g., in a coding region or in a non-coding region). Both approaches give rise to alteration of the CCR5 gene as described herein. In an embodiment, a CCR5 target knockout position is altered by genome editing using the CRISPR/Cas9 system. The CCR5 target knockout position may be targeted by cleaving with either one or more nucleases, or one or more nickases, or a combination thereof.

“CCR5 target knockout position”, as used herein, refers to a position in the CCR5 gene, which if altered, e.g., disrupted by insertion or deletion of one or more nucleotides, e.g., by NHEJ-mediated alteration, results in alteration of the CCR5 gene. In an embodiment, the position is in the CCR5 coding region, e.g., an early coding region. In another embodiment, the position is in a non-coding sequence of the CCR5 gene, e.g., a promoter, an enhancer, an intron, a 3′UTR, and/or a polyadenylation signal.

In another embodiment, the CCR5 gene is targeted to knock down the gene, e.g., to reduce or eliminate expression of the gene, e.g., to knock down one or both alleles of the CCR5 gene.

In one embodiment, the coding region of the CCR5 gene, is targeted to alter the expression of the gene. In another embodiment, a non-coding region (e.g., an enhancer region, a promoter region, an intron, a 5′ UTR, a 3′UTR, or a polyadenylation signal) of the CCR5 gene is targeted to alter the expression of the gene. In an embodiment, the promoter region of the CCR5 gene is targeted to knock down the expression of the CCR5 gene. This type of alteration is also sometimes referred to as “knocking down” the CCR5 gene. While not wishing to be bound by theory, in an embodiment, a targeted knockdown approach is mediated by a CRISPR/Cas system comprising a Cas9 molecule, e.g., an enzymatically inactive Cas9 (eiCas9) molecule or an eiCas9 fusion protein (e.g., an eiCas9 fused to a transcription repressor domain or chromatin modifying protein), as described herein. In an embodiment, the CCR5 gene is targeted to alter (e.g., to block, reduce, or decrease) the transcription of the CCR5 gene. In another embodiment, the CCR5 gene is targeted to alter the chromatin structure (e.g., one or more histone and/or DNA modifications) of the CCR5 gene. In an embodiment, a CCR5 target knockdown position is targeted by genome editing using the CRISPR/Cas9 system. In an embodiment, one or more gRNA molecules comprising a targeting domain are configured to target an enzymatically inactive Cas9 (eiCas9) molecule or an eiCas9 fusion protein (e.g., an eiCas9 fused to a transcription repressor domain), sufficiently close to a CCR5 target knockdown position to reduce, decrease or repress expression of the CCR5 gene.

“CCR5 target knockdown position”, as used herein, refers to a position in the CCR5 gene, which if targeted, e.g., by an eiCas9 molecule or an eiCas9 fusion described herein, results in reduction or elimination of expression of functional CCR5 gene product. In an embodiment, the transcription of the CCR5 gene is reduced or eliminated. In another embodiment, the chromatin structure of the CCR5 gene is altered. In an embodiment, the position is in the CCR5 promoter sequence. In an embodiment, a position in the promoter sequence of the CCR5 gene is targeted by an enzymatically inactive Cas9 (eiCas9) molecule or an eiCas9 fusion protein, as described herein.

“CCR5 target position”, as used herein, refers to any position that results in inactivation of the CCR5 gene. In an embodiment, a CCR5 target position refers to any of a CCR5 target knockout position or a CCR5 target knockdown position, as described herein.

In one aspect, disclosed herein is a gRNA molecule, e.g., an isolated or non-naturally occurring gRNA molecule, comprising a targeting domain which is complementary with a target domain from the CCR5 gene.

In an embodiment, the targeting domain of the gRNA molecule is configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene. In an embodiment, the alteration comprises an insertion or deletion. In an embodiment, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 400, 450, or 500 nucleotides of a CCR5 target position. The break, e.g., a double strand or single strand break, can be positioned upstream or downstream of a CCR5 target position in the CCR5 gene.

In an embodiment, a second gRNA molecule comprising a second targeting domain is configured to provide a cleavage event, e.g., a double strand break or a single strand break, sufficiently close to the CCR5 target position in the CCR5 gene, to allow alteration, e.g., alteration associated with NHEJ, of the CCR5 target position in the CCR5 gene, either alone or in combination with the break positioned by said first gRNA molecule. In an embodiment, the targeting domains of the first and second gRNA molecules are configured such that a cleavage event, e.g., a double strand or single strand break, is positioned, independently for each of the gRNA molecules, within 1, 2, 3, 4, 5, 10, 15, 20, 25, 30, 35, 40, 45, 50, 60, 70, 80, 90, 100, 150, 200, 300, 400, 450, or 500 nucleotides of the target position. In an embodiment, the breaks, e.g., double strand or single strand breaks, are positioned on both sides of a nucleotide of a CCR5 target position in the CCR5 gene. In an embodiment, the breaks, e.g., double strand or single strand breaks, are positioned on one side, e.g., upstream or downstream, of a nucleotide of a CCR5 target position in the CCR5 gene.

In an embodiment, a single strand break is accompanied by an additional single strand break, positioned by a second gRNA molecule, as discussed below. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 400, 450, or 500 nucleotides of a CCR5 target position. In an embodiment, the first and second gRNA molecules are configured such, that when guiding a Cas9 molecule, e.g., a Cas9 nickase, a single strand break will be accompanied by an additional single strand break, positioned by a second gRNA, sufficiently close to one another to result in alteration of a CCR5 target position in the CCR5 gene. In an embodiment, the first and second gRNA molecules are configured such that a single strand break positioned by said second gRNA is within 10, 20, 30, 40, or 50 nucleotides of the break positioned by said first gRNA molecule, e.g., when the Cas9 molecule is a nickase. In an embodiment, the two gRNA molecules are configured to position cuts at the same position, or within a few nucleotides of one another, on different strands, e.g., essentially mimicking a double strand break.

In an embodiment, a double strand break can be accompanied by an additional double strand break, positioned by a second gRNA molecule, as is discussed below. For example, the targeting domain of a first gRNA molecule is configured such that a double strand break is positioned upstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domain of a second gRNA molecule is configured such that a double strand break is positioned downstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position.

In an embodiment, a double strand break can be accompanied by two additional single strand breaks, positioned by a second gRNA molecule and a third gRNA molecule. For example, the targeting domain of a first gRNA molecule is configured such that a double strand break is positioned upstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domains of a second and third gRNA molecule are configured such that two single strand breaks are positioned downstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In an embodiment, the targeting domain of the first, second and third gRNA molecules are configured such that a cleavage event, e.g., a double strand or single strand break, is positioned, independently for each of the gRNA molecules.

In an embodiment, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domains of a third and fourth gRNA molecule are configured such that two single strand breaks are positioned downstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position.

It is contemplated herein that, in an embodiment, when multiple gRNAs are used to generate (1) two single stranded breaks in close proximity, (2) two double stranded breaks, e.g., flanking a CCR5 target position (e.g., to remove a piece of DNA, e.g., a insertion or deletion mutation) or to create more than one indel in an early coding region, (3) one double stranded break and two paired nicks flanking a CCR5 target position (e.g., to remove a piece of DNA, e.g., a insertion or deletion mutation) or (4) four single stranded breaks, two on each side of a CCR5 target position, that they are targeting the same CCR5 target position. It is further contemplated herein that in an embodiment multiple gRNAs may be used to target more than one target position in the same gene.

In an embodiment, the targeting domain of the first gRNA molecule and the targeting domain of the second gRNA molecules are complementary to opposite strands of the target nucleic acid molecule. In an embodiment, the gRNA molecule and the second gRNA molecule are configured such that the PAMs are oriented outward.

In an embodiment, the targeting domain of a gRNA molecule is configured to avoid unwanted target chromosome elements, such as repeat elements, e.g., Alu repeats, in the target domain. The gRNA molecule may be a first, second, third and/or fourth gRNA molecule, as described herein.

In an embodiment, the targeting domain of a gRNA molecule is configured to position a cleavage event sufficiently far from a preselected nucleotide, e.g., the nucleotide of a coding region, such that the nucleotide is not altered. In an embodiment, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

In an embodiment, a CCR5 target position is targeted and the targeting domain of a gRNA molecule comprises a sequence that is the same as, or differs by no more than 1, 2, 3, 4, or 5 nucleotides from, a targeting domain sequence from any one of Tables 1A-1F, 2A-2C, 3A-3E, or 4A-4C. In an embodiment, the targeting domain is independently selected from those in Tables 1A-1F, 2A-2C, 3A-3E, or 4A-4C. In an embodiment, the targeting domain is independently selected from:

(SEQ ID NO: 387)

CCUGCCUCCGCUCUACUCAC;

(SEQ ID NO: 388)

GCUGCCGCCCAGUGGGACUU;

(SEQ ID NO: 389)

ACAAUGUGUCAACUCUUGAC;

(SEQ ID NO: 390)

GGUGACAAGUGUGAUCACUU;

(SEQ ID NO: 391)

CCAGGUACCUAUCGAUUGUC;

(SEQ ID NO: 392)

CUUCACAUUGAUUUUUUGGC;

(SEQ ID NO: 393)

GCAGCAUAGUGAGCCCAGAA;

(SEQ ID NO: 394)

GGUACCUAUCGAUUGUCAGG;

(SEQ ID NO: 395)

GUGAGUAGAGCGGAGGCAGG;

(SEQ ID NO: 396)

GCCUCCGCUCUACUCAC;

(SEQ ID NO: 397)

GCCGCCCAGUGGGACUU;

(SEQ ID NO: 398)

AUGUGUCAACUCUUGAC;

(SEQ ID NO: 399)

GACAAUCGAUAGGUACC;

(SEQ ID NO: 400)

CACAUUGAUUUUUUGGC;

(SEQ ID NO: 401)

GCAUAGUGAGCCCAGAA;
or

(SEQ ID NO: 402)

GGUACCUAUCGAUUGUC.

In an embodiment, the targeting domain is independently selected from those in Table 2A. In an embodiment, the targeting domain is independently selected from those in Table 3A. In an embodiment, the targeting domain is independently selected from those in Table 4A.

In an embodiment, more than one gRNA is used to position breaks, e.g., two single stranded breaks or two double stranded breaks, or a combination of single strand and double strand breaks, e.g., to create one or more indels, in the target nucleic acid sequence. In an embodiment, the targeting domain of each guide RNA is independently selected from any one of Tables 1A-1F, 2A-2C, 3A-3E, or 4A-4C.

In an embodiment, the targeting domain of the gRNA molecule is configured to target an enzymatically inactive Cas9 (eiCas9) molecule or an eiCas9 fusion protein (e.g., an eiCas9 fused to a transcription repressor domain), sufficiently close to a CCR5 transcription start site (TSS) to reduce (e.g., block) transcription, e.g., transcription initiation or elongation, binding of one or more transcription enhancers or activators, and/or RNA polymerase. In an embodiment, the targeting domain is configured to target between 1000 bp upstream and 1000 bp downstream (e.g., between 500 bp upstream and 1000 bp downstream, between 1000 bp upstream and 500 bp downstream, between 500 bp upstream and 500 bp downstream, within 500 bp or 200 bp upstream, or within 500 bp or 200 bp downstream) of the TSS of the CCR5 gene. One or more gRNAs may be used to target an eiCas9 to the promoter region of the CCR5 gene.

In an embodiment, the targeting domain comprises a sequence that is the same as, or differs by no more than 1, 2, 3, 4, or 5 nucleotides from, a targeting domain sequence from any one of Tables 5A-5C, 6A-6E, or 7A-7C. In an embodiment, the targeting domain is independently selected from those in Tables 5A-5C, 6A-6E, or 7A-7C.

In an embodiment, the targeting domain is independently selected from those in Table 5A. In an embodiment, the targeting domain is independently selected from those in Table 6A. In an embodiment, the targeting domain is independently selected from those in Table 7A.

In an embodiment, when the CCR5 promoter region is targeted, e.g., for knockdown, the targeting domain can comprise a sequence that is the same as, or differs by no more than 1, 2, 3, 4, or 5 nucleotides from, a targeting domain sequence from any one of Tables 5A-5C, 6A-6E, or 7A-7C. In an embodiment, the targeting domain is independently selected from those in Tables 5A-5C, 6A-6E, or 7A-7C.

In an embodiment, when the CCR5 target knockdown position is the CCR5 promoter region and more than one gRNA is used to position an eiCas9 molecule or an eiCas9-fusion protein (e.g., an eiCas9-transcription repressor domain fusion protein), in the target nucleic acid sequence, the targeting domain for each guide RNA is independently selected from one of Tables 5A-5C, 6A-6E, or 7A-7C.

In an embodiment, the targeting domain comprises a sequence that is the same as, or differs by no more than 1, 2, 3, 4, or 5 nucleotides from, a targeting domain sequence from Table 18. In an embodiment, the targeting domain is independently selected from those in Table 18.

In an embodiment, the targeting domain which is complementary with a target domain from the CCR5 target position in the CCR5 gene is 16 nucleotides or more in length. In an embodiment, the targeting domain is 16 nucleotides in length. In an embodiment, the targeting domain is 17 nucleotides in length. In other embodiments, the targeting domain is 18 nucleotides in length. In still other embodiments, the targeting domain is 19 nucleotides in length. In still other embodiments, the targeting domain is 20 nucleotides in length. In an embodiment, the targeting domain is 21 nucleotides in length. In an embodiment, the targeting domain is 22 nucleotides in length. In an embodiment, the targeting domain is 23 nucleotides in length. In an embodiment, the targeting domain is 24 nucleotides in length. In an embodiment, the targeting domain is 25 nucleotides in length. In an embodiment, the targeting domain is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

A gRNA as described herein may comprise from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a gRNA comprises a linking domain of no more than 25 nucleotides in length; a proximal and tail domain, that taken together, are at least 20 nucleotides in length; and a targeting domain equal to or greater than 16, 17, 18, 19, 20, 21, 22, 23, 24, 25 or 26 nucleotides in length.

In another embodiment, a gRNA comprises a linking domain of no more than 25 nucleotides in length; a proximal and tail domain, that taken together, are at least 25 nucleotides in length; and a targeting domain equal to or greater than 16, 17, 18, 19, 20, 21, 22, 23, 24, 25 or 26 nucleotides in length.

In another embodiment, a gRNA comprises a linking domain of no more than 25 nucleotides in length; a proximal and tail domain, that taken together, are at least 30 nucleotides in length; and a targeting domain equal to or greater than 16, 17, 18, 19, 20, 21, 22, 23, 24, 25 or 26 nucleotides in length.

In another embodiment, a gRNA comprises a linking domain of no more than 25 nucleotides in length; a proximal and tail domain, that taken together, are at least 40 nucleotides in length; and a targeting domain equal to or greater than 16, 17, 18, 19, 20, 21, 22, 23, 24, 25 or 26 nucleotides in length.

A cleavage event, e.g., a double strand or single strand break, is generated by a Cas9 molecule. The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule).

In an embodiment, the eaCas9 molecule catalyzes a double strand break.

In some embodiments, the eaCas9 molecule comprises HNH-like domain cleavage activity but has no, or no significant, N-terminal RuvC-like domain cleavage activity. In this case, the eaCas9 molecule is an HNH-like domain nickase, e.g., the eaCas9 molecule comprises a mutation at D10, e.g., D10A. In other embodiments, the eaCas9 molecule comprises N-terminal RuvC-like domain cleavage activity but has no, or no significant, HNH-like domain cleavage activity. In an embodiment, the eaCas9 molecule is an N-terminal RuvC-like domain nickase, e.g., the eaCas9 molecule comprises a mutation at H840, e.g., H840A. In an embodiment, the eaCas9 molecule is an N-terminal RuvC-like domain nickase, e.g., the eaCas9 molecule comprises a mutation at N863, e.g., N863A.

In an embodiment, a single strand break is formed in the strand of the target nucleic acid to which the targeting domain of said gRNA is complementary. In another embodiment, a single strand break is formed in the strand of the target nucleic acid other than the strand to which the targeting domain of said gRNA is complementary.

In another aspect, disclosed herein is a nucleic acid, e.g., an isolated or non-naturally occurring nucleic acid, e.g., DNA, that comprises (a) a sequence that encodes a gRNA molecule comprising a targeting domain that is complementary with a CCR5 target position in the CCR5 gene as disclosed herein.

In an embodiment, the nucleic acid encodes a gRNA molecule, e.g., a first gRNA molecule, comprising a targeting domain configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene.

In an embodiment, the nucleic acid encodes a gRNA molecule, e.g., a first gRNA molecule, comprising a targeting domain configured to target an enzymatically inactive Cas9 (eiCas9) molecule or an eiCas9 fusion protein (e.g., an eiCas9 fused to a transcription repressor domain or chromatin modifying protein), sufficiently close to a CCR5 knockdown target position to reduce, decrease or repress expression of the CCR5 gene.

In an embodiment, the nucleic acid encodes a gRNA molecule, e.g., the first gRNA molecule, comprising a targeting domain comprising a sequence that is the same as, or differs by no more than 1, 2, 3, 4, or 5 nucleotides from, a targeting domain sequence from any one of Tables 1A-1F, 2A-2C, 3A-3E, 4A-4C, 5A-5C, 6A-6E, 7A-7C, or 18. In an embodiment, the nucleic acid encodes a gRNA molecule comprising a targeting domain is selected from those in Tables 1A-1F, 2A-2C, 3A-3E, 4A-4C, 5A-5C, 6A-6E, 7A-7C, or 18.

In an embodiment, the nucleic acid encodes a gRNA molecule, e.g., the first gRNA molecule, comprising a targeting domain comprising a sequence that is the same as, or differs by no more than 1, 2, 3, 4, or 5 nucleotides from, a targeting domain sequence from any one of Tables 1A-1F, 2A-2C, 3A-3E, or 4A-4C. In an embodiment, the nucleic acid encodes a gRNA molecule comprising a targeting domain is selected from those in Tables 1A-1F, 2A-2C, 3A-3E, or 4A-4C.

In an embodiment, the nucleic acid encodes a gRNA molecule, e.g., the first gRNA molecule, comprising a targeting domain comprising a sequence that is the same as, or differs by no more than 1, 2, 3, 4, or 5 nucleotides from, a targeting domain sequence from any one of Tables 5A-5C, 6A-6E, or 7A-7C. In an embodiment, the nucleic acid encodes a gRNA molecule comprising a targeting domain is selected from those in Tables 5A-5C, 6A-6E, or 7A-7C.

In an embodiment, the nucleic acid encodes a modular gRNA, e.g., one or more nucleic acids encode a modular gRNA. In other embodiments, the nucleic acid encodes a chimeric gRNA. The nucleic acid may encode a gRNA, e.g., the first gRNA molecule, comprising a targeting domain comprising 16 nucleotides or more in length. In an embodiment, the nucleic acid encodes a gRNA, e.g., the first gRNA molecule, comprising a targeting domain that is 16 nucleotides in length. In another embodiment, the nucleic acid encodes a gRNA, e.g., the first gRNA molecule, comprising a targeting domain that is 17 nucleotides in length. In yet another embodiment, the nucleic acid encodes a gRNA, e.g., the first gRNA molecule, comprising a targeting domain that is 18 nucleotides in length. In still another embodiment, the nucleic acid encodes a gRNA, e.g., the first gRNA molecule, comprising a targeting domain that is 19 nucleotides in length. In still another embodiment, the nucleic acid encodes a gRNA, e.g., the first gRNA molecule, comprising a targeting domain that is 20 nucleotides in length. In still another embodiment, the nucleic acid encodes a gRNA, e.g., the first gRNA molecule, comprising a targeting domain that is 21 nucleotides in length. In still another embodiment, the nucleic acid encodes a gRNA, e.g., the first gRNA molecule, comprising a targeting domain that is 22 nucleotides in length. In still another embodiment, the nucleic acid encodes a gRNA, e.g., the first gRNA molecule, comprising a targeting domain that is 23 nucleotides in length. In still another embodiment, the nucleic acid encodes a gRNA, e.g., the first gRNA molecule, comprising a targeting domain that is 24 nucleotides in length. In still another embodiment, the nucleic acid encodes a gRNA, e.g., the first gRNA molecule, comprising a targeting domain that is 25 nucleotides in length. In still another embodiment, the nucleic acid encodes a gRNA, e.g., the first gRNA molecule, comprising a targeting domain that is 26 nucleotides in length. In an embodiment, a nucleic acid encodes a gRNA comprising from 5′ to 3′: a targeting domain (comprising a “core domain”, and optionally a “secondary domain”); a first complementarity domain; a linking domain; a second complementarity domain; a proximal domain; and a tail domain. In an embodiment, the proximal domain and tail domain are taken together as a single domain.

In an embodiment, a nucleic acid encodes a gRNA e.g., the first gRNA molecule, comprising a linking domain of no more than 25 nucleotides in length; a proximal and tail domain, that taken together, are at least 20 nucleotides in length; and a targeting domain equal to or greater than 16, 17, 18, 19, 20, 21, 22, 23, 24, 25 or 26 nucleotides in length.

In an embodiment, a nucleic acid encodes a gRNA e.g., the first gRNA molecule, comprising a linking domain of no more than 25 nucleotides in length; a proximal and tail domain, that taken together, are at least 25 nucleotides in length; and a targeting equal to or greater than 16, 17, 18, 19, 20, 21, 22, 23, 24, 25 or 26 nucleotides in length.

In an embodiment, a nucleic acid encodes a gRNA e.g., the first gRNA molecule, comprising a linking domain of no more than 25 nucleotides in length; a proximal and tail domain, that taken together, are at least 30 nucleotides in length; and a targeting domain equal to or greater than 16, 17, 18, 19, 20, 21, 22, 23, 24, 25 or 26 nucleotides in length.

In an embodiment, a nucleic acid encodes a gRNA comprising e.g., the first gRNA molecule, a linking domain of no more than 25 nucleotides in length; a proximal and tail domain, that taken together, are at least 40 nucleotides in length; and a targeting domain equal to or greater than 16, 17, 18, 19, 20, 21, 22, 23, 24, 25 or 26 nucleotides in length.

In an embodiment, a nucleic acid comprises (a) a sequence that encodes a gRNA molecule e.g., the first gRNA molecule, comprising a targeting domain that is complementary with a target domain in the CCR5 gene as disclosed herein, and further comprising (b) a sequence that encodes a Cas9 molecule.

The Cas9 molecule may be a nickase molecule, an enzymatically active Cas9 (eaCas9) molecule, e.g., an eaCas9 molecule that forms a double strand break in a target nucleic acid and/or an eaCas9 molecule that forms a single strand break in a target nucleic acid. In an embodiment, a single strand break is formed in the strand of the target nucleic acid to which the targeting domain of said gRNA is complementary. In another embodiment, a single strand break is formed in the strand of the target nucleic acid other than the strand to which to which the targeting domain of said gRNA is complementary.

In an embodiment, the eaCas9 molecule catalyzes a double strand break.

In an embodiment, the eaCas9 molecule comprises HNH-like domain cleavage activity but has no, or no significant, N-terminal RuvC-like domain cleavage activity. In another embodiment, the said eaCas9 molecule is an HNH-like domain nickase, e.g., the eaCas9 molecule comprises a mutation at D10, e.g., D10A. In another embodiment, the eaCas9 molecule comprises N-terminal RuvC-like domain cleavage activity but has no, or no significant, HNH-like domain cleavage activity. In another embodiment, the eaCas9 molecule is an N-terminal RuvC-like domain nickase, e.g., the eaCas9 molecule comprises a mutation at H840, e.g., H840A. In another embodiment, the eaCas9 molecule is an N-terminal RuvC-like domain nickase, e.g., the eaCas9 molecule comprises a mutation at N863, e.g., N863A.

A nucleic acid disclosed herein may comprise (a) a sequence that encodes a gRNA molecule comprising a targeting domain that is complementary with a target domain in the CCR5 gene as disclosed herein; (b) a sequence that encodes a Cas9 molecule, e.g., a Cas9 molecule described herein.

In an embodiment, the Cas9 molecule is an enzymatically active Cas9 (eaCas9) molecule. In an embodiment, the Cas9 molecule is an enzymatically inactive Cas9 (eiCas9) molecule or a modified eiCas9 molecule, e.g., the eiCas9 molecule is fused to Krüppel-associated box (KRAB) to generate an eiCas9-KRAB fusion protein molecule.

A nucleic acid disclosed herein may comprise (a) a sequence that encodes a gRNA molecule comprising a targeting domain that is complementary with a target domain in the CCR5 gene as disclosed herein; (b) a sequence that encodes a Cas9 molecule; and further may comprise (c)(i) a sequence that encodes a second gRNA molecule described herein having a targeting domain that is complementary to a second target domain of the CCR5 gene, and optionally, (c)(ii) a sequence that encodes a third gRNA molecule described herein having a targeting domain that is complementary to a third target domain of the CCR5 gene; and optionally, (c)(iii) a sequence that encodes a fourth gRNA molecule described herein having a targeting domain that is complementary to a fourth target domain of the CCR5 gene.

In an embodiment, a nucleic acid encodes a second gRNA molecule comprising a targeting domain configured to provide a cleavage event, e.g., a double strand break or a single strand break, sufficiently close to a CCR5 target position in the CCR5 gene, to allow alteration, e.g., alteration associated with NHEJ, of a CCR5 target position in the CCR5 gene, either alone or in combination with the break positioned by said first gRNA molecule.

In an embodiment, a nucleic acid encodes a second gRNA molecule comprising a targeting domain configured to target an enzymatically inactive Cas9 (eiCas9) molecule or an eiCas9 fusion protein (e.g., an eiCas9 fused to a transcription repressor domain or chromatin modifying protein), sufficiently close to a CCR5 knockdown target position to reduce, decrease or repress expression of the CCR5 gene.

In an embodiment, a nucleic acid encodes a third gRNA molecule comprising a targeting domain configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene, either alone or in combination with the break positioned by the first and/or second gRNA molecule.

In an embodiment, a nucleic acid encodes a third gRNA molecule comprising a targeting domain configured to target an enzymatically inactive Cas9 (eiCas9) molecule or an eiCas9 fusion protein (e.g., an eiCas9 fused to a transcription repressor domain or chromatin remodeling protein), sufficiently close to a CCR5 knockdown target position to reduce, decrease or repress expression of the CCR5 gene.

In an embodiment, a nucleic acid encodes a fourth gRNA molecule comprising a targeting domain configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene, either alone or in combination with the break positioned by the first gRNA molecule, the second gRNA molecule and/or the third gRNA molecule.

In an embodiment, the nucleic acid encodes a second gRNA molecule. The second gRNA is selected to target the same CCR5 target position as the first gRNA molecule. Optionally, the nucleic acid may encode a third gRNA, and further optionally, the nucleic acid may encode a fourth gRNA molecule. The third gRNA molecule and the fourth gRNA molecule are selected to target the same CCR5 target position as the first and second gRNA molecules.

In an embodiment, the nucleic acid encodes a second gRNA molecule comprising a targeting domain comprising a sequence that is the same as, or differs by no more than 1, 2, 3, 4, or 5 nucleotides from, a targeting domain sequence from one of Tables 1A-1F, 2A-2C, 3A-3E, 4A-4C, 5A-5C, 6A-6E, 7A-7C, or 18. In an embodiment, the nucleic acid encodes a second gRNA molecule comprising a targeting domain selected from those in Tables 1A-1F, 2A-2C, 3A-3E, 4A-4C, 5A-5C, 6A-6E, 7A-7C, or 18. In an embodiment, when a third or fourth gRNA molecule are present, the third and fourth gRNA molecules may independently comprise a targeting domain comprising a sequence that is the same as, or differs by no more than 1, 2, 3, 4, or 5 nucleotides from, a targeting domain sequence from one of Tables 1A-1F, 2A-2C, 3A-3E, 4A-4C, 5A-5C, 6A-6E, 7A-7C, or 18. In a further embodiment, when a third or fourth gRNA molecule are present, the third and fourth gRNA molecules may independently comprise a targeting domain selected from those in Tables 1A-1F, 2A-2C, 3A-3E, 4A-4C, 5A-5C, 6A-6E, 7A-7C, or 18.

In an embodiment, the nucleic acid encodes a second gRNA molecule comprising a targeting domain comprising a sequence that is the same as, or differs by no more than 1, 2, 3, 4, or 5 nucleotides from, a targeting domain sequence from one of Tables 1A-1F, 2A-2C, 3A-3E, or 4A-4C. In an embodiment, the nucleic acid encodes a second gRNA molecule comprising a targeting domain selected from those in Tables 1A-1F, 2A-2C, 3A-3E, or 4A-4C. In an embodiment, when a third or fourth gRNA molecule are present, the third and fourth gRNA molecules may independently comprise a targeting domain comprising a sequence that is the same as, or differs by no more than 1, 2, 3, 4, or 5 nucleotides from, a targeting domain sequence from one of Tables 1A-1F, 2A-2C, 3A-3E, or 4A-4C. In a further embodiment, when a third or fourth gRNA molecule are present, the third and fourth gRNA molecules may independently comprise a targeting domain selected from those in Tables 1A-1F, 2A-2C, 3A-3E, or 4A-4C.

In an embodiment, the nucleic acid encodes a second gRNA molecule comprising a targeting domain comprising a sequence that is the same as, or differs by no more than 1, 2, 3, 4, or 5 nucleotides from, a targeting domain sequence from one of Tables 5A-5C, 6A-6E, or 7A-7C. In an embodiment, the nucleic acid encodes a second gRNA molecule comprising a targeting domain selected from those in Tables 5A-5C, 6A-6E, or 7A-7C. In an embodiment, when a third or fourth gRNA molecule are present, the third and fourth gRNA molecules may independently comprise a targeting domain comprising a sequence that is the same as, or differs by no more than 1, 2, 3, 4, or 5 nucleotides from, a targeting domain sequence from one of Tables 5A-5C, 6A-6E, or 7A-7C. In a further embodiment, when a third or fourth gRNA molecule are present, the third and fourth gRNA molecules may independently comprise a targeting domain selected from those in Tables 5A-5C, 6A-6E, or 7A-7C.

In an embodiment, the nucleic acid encodes a second gRNA which is a modular gRNA, e.g., wherein one or more nucleic acid molecules encode a modular gRNA. In another embodiment, the nucleic acid encoding a second gRNA is a chimeric gRNA. In yet another embodiment, when a nucleic acid encodes a third or fourth gRNA, the third and fourth gRNA may be a modular gRNA or a chimeric gRNA. When multiple gRNAs are used, any combination of modular or chimeric gRNAs may be used.

A nucleic acid may encode a second, a third, and/or a fourth gRNA, each independently, comprising a targeting domain comprising 16 nucleotides or more in length. In an embodiment, the nucleic acid encodes a second gRNA comprising a targeting domain that is 16 nucleotides in length. In another embodiment, the nucleic acid encodes a second gRNA comprising a targeting domain that is 17 nucleotides in length. In yet another embodiment, the nucleic acid encodes a second gRNA comprising a targeting domain that is 18 nucleotides in length. In still another embodiment, the nucleic acid encodes a second gRNA comprising a targeting domain that is 19 nucleotides in length. In still other embodiments, the nucleic acid encodes a second gRNA comprising a targeting domain that is 20 nucleotides in length. In still another embodiment, the nucleic acid encodes a second gRNA comprising a targeting domain that is 21 nucleotides in length. In still another embodiment, the nucleic acid encodes a second gRNA comprising a targeting domain that is 22 nucleotides in length. In still another embodiment, the nucleic acid encodes a second gRNA comprising a targeting domain that is 23 nucleotides in length. In still another embodiment, the nucleic acid encodes a second gRNA comprising a targeting domain that is 24 nucleotides in length. In still another embodiment, the nucleic acid encodes a second gRNA comprising a targeting domain that is 25 nucleotides in length. In still another embodiment, the nucleic acid encodes a second gRNA comprising a targeting domain that is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

In an embodiment, a nucleic acid encodes a second, a third, and/or a fourth gRNA, each independently, comprising from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a nucleic acid encodes a second, a third, and/or a fourth gRNA comprising a linking domain of no more than 25 nucleotides in length; a proximal and tail domain, that taken together, are at least 20 nucleotides in length; and a targeting domain equal to or greater than 16, 17, 18, 19, 20, 21, 22, 23, 24, 25 or 26 nucleotides in length.

In an embodiment, a nucleic acid encodes a second, a third, and/or a fourth gRNA comprising a linking domain of no more than 25 nucleotides in length; a proximal and tail domain, that taken together, are at least 25 nucleotides in length; and a targeting domain equal to or greater than 16, 17, 18, 19, 20, 21, 22, 23, 24, 25 or 26 nucleotides in length.

In an embodiment, a nucleic acid encodes a second, a third, and/or a fourth gRNA comprising a linking domain of no more than 25 nucleotides in length; a proximal and tail domain, that taken together, are at least 30 nucleotides in length; and a targeting domain equal to or greater than 16, 17, 18, 19, 20, 21, 22, 23, 24, 25 or 26 nucleotides in length.

In an embodiment, a nucleic acid encodes a second, a third, and/or a fourth gRNA comprising a linking domain of no more than 25 nucleotides in length; a proximal and tail domain, that taken together, are at least 40 nucleotides in length; and a targeting domain equal to or greater than 16, 17, 18, 19, 20, 21, 22, 23, 24, 25 or 26 nucleotides in length.

In an embodiment, a nucleic acid encodes (a) a sequence that encodes a gRNA molecule comprising a targeting domain that is complementary with a target domain in the CCR5 gene as disclosed herein, and (b) a sequence that encodes a Cas9 molecule, e.g., a Cas9 molecule described herein. In an embodiment, (a) and (b) are present on the same nucleic acid molecule, e.g., the same vector, e.g., the same viral vector, e.g., the same adeno-associated virus (AAV) vector. In an embodiment, the nucleic acid molecule is an AAV vector. Exemplary AAV vectors that may be used in any of the described compositions and methods include an AAV1 vector, a modified AAV1 vector, an AAV2 vector, a modified AAV2 vector, an AAV3 vector, an AAV4 vector, a modified AAV4 vector, an AAV5 vector, a modified AAV5 vector, a modified AAV3 vector, an AAV6 vector, a modified AAV6 vector, an AAV8 vector an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector.

In another embodiment, (a) is present on a first nucleic acid molecule, e.g. a first vector, e.g., a first viral vector, e.g., a first AAV vector; and (b) is present on a second nucleic acid molecule, e.g., a second vector, e.g., a second vector, e.g., a second AAV vector. The first and second nucleic acid molecules may be AAV vectors.

In another embodiment, a nucleic acid encodes (a) a sequence that encodes a gRNA molecule comprising a targeting domain that is complementary with a target domain in the CCR5 gene as disclosed herein, and (b) a sequence that encodes a Cas9 molecule, e.g., a Cas9 molecule described herein; and further comprises (c)(i) a sequence that encodes a second gRNA molecule as described herein and optionally, (c)(ii) a sequence that encodes a third gRNA molecule described herein having a targeting domain that is complementary to a third target domain of the CCR5 gene; and optionally, (c)(iii) a sequence that encodes a fourth gRNA molecule described herein having a targeting domain that is complementary to a fourth target domain of the CCR5 gene. In an embodiment, the nucleic acid comprises (a), (b) and (c)(i). In an embodiment, the nucleic acid comprises (a), (b), (c)(i) and (c)(ii). In an embodiment, the nucleic acid comprises (a), (b), (c)(i), (c)(ii) and (c)(iii). Each of (a) and (c)(i) may be present on the same nucleic acid molecule, e.g., the same vector, e.g., the same viral vector, e.g., the same adeno-associated virus (AAV) vector. In an embodiment, the nucleic acid molecule is an AAV vector.

In an embodiment, (a) and (c)(i) are on different vectors. For example, (a) may be present on a first nucleic acid molecule, e.g. a first vector, e.g., a first viral vector, e.g., a first AAV vector; and (c)(i) may be present on a second nucleic acid molecule, e.g., a second vector, e.g., a second vector, e.g., a second AAV vector. In an embodiment, the first and second nucleic acid molecules are AAV vectors.

In another embodiment, each of (a), (b), and (c)(i) are present on the same nucleic acid molecule, e.g., the same vector, e.g., the same viral vector, e.g., an AAV vector. In an embodiment, the nucleic acid molecule is an AAV vector. In an alternate embodiment, one of (a), (b), and (c)(i) is encoded on a first nucleic acid molecule, e.g., a first vector, e.g., a first viral vector, e.g., a first AAV vector; and a second and third of (a), (b), and (c)(i) is encoded on a second nucleic acid molecule, e.g., a second vector, e.g., a second vector, e.g., a second AAV vector. The first and second nucleic acid molecule may be AAV vectors.

In an embodiment, (a) is present on a first nucleic acid molecule, e.g., a first vector, e.g., a first viral vector, a first AAV vector; and (b) and (c)(i) are present on a second nucleic acid molecule, e.g., a second vector, e.g., a second vector, e.g., a second AAV vector. The first and second nucleic acid molecule may be AAV vectors.

In another embodiment, (b) is present on a first nucleic acid molecule, e.g., a first vector, e.g., a first viral vector, e.g., a first AAV vector; and (a) and (c)(i) are present on a second nucleic acid molecule, e.g., a second vector, e.g., a second vector, e.g., a second AAV vector. The first and second nucleic acid molecule may be AAV vectors.

In another embodiment, (c)(i) is present on a first nucleic acid molecule, e.g., a first vector, e.g., a first viral vector, e.g., a first AAV vector; and (b) and (a) are present on a second nucleic acid molecule, e.g., a second vector, e.g., a second vector, e.g., a second AAV vector. The first and second nucleic acid molecule may be AAV vectors.

In another embodiment, each of (a), (b) and (c)(i) are present on different nucleic acid molecules, e.g., different vectors, e.g., different viral vectors, e.g., different AAV vector. For example, (a) may be on a first nucleic acid molecule, (b) on a second nucleic acid molecule, and (c)(i) on a third nucleic acid molecule. The first, second and third nucleic acid molecule may be AAV vectors.

In another embodiment, when a third and/or fourth gRNA molecule are present, each of (a), (b), (c)(i), (c)(ii) and (c)(iii) may be present on the same nucleic acid molecule, e.g., the same vector, e.g., the same viral vector, e.g., an AAV vector. In an embodiment, the nucleic acid molecule is an AAV vector. In an alternate embodiment, each of (a), (b), (c)(i), (c)(ii) and (c)(iii) may be present on the different nucleic acid molecules, e.g., different vectors, e.g., the different viral vectors, e.g., different AAV vectors. In a further embodiment, each of (a), (b), (c)(i), (c)(ii) and (c)(iii) may be present on more than one nucleic acid molecule, but fewer than five nucleic acid molecules, e.g., AAV vectors.

The nucleic acids described herein may comprise a promoter operably linked to the sequence that encodes the gRNA molecule of (a), e.g., a promoter described herein. The nucleic acid may further comprise a second promoter operably linked to the sequence that encodes the second, third and/or fourth gRNA molecule of (c), e.g., a promoter described herein. The promoter and second promoter differ from one another. In some embodiments, the promoter and second promoter are the same.

The nucleic acids described herein may further comprise a promoter operably linked to the sequence that encodes the Cas9 molecule of (b), e.g., a promoter described herein.

In another aspect, disclosed herein is a composition comprising (a) a gRNA molecule comprising a targeting domain that is complementary with a target domain in the CCR5 gene, as described herein. The composition of (a) may further comprise (b) a Cas9 molecule, e.g., a Cas9 molecule as described herein. A composition of (a) and (b) may further comprise (c) a second, third and/or fourth gRNA molecule, e.g., a second, third and/or fourth gRNA molecule described herein. In an embodiment, the composition is a pharmaceutical composition. The compositions described herein, e.g., pharmaceutical compositions described herein, can be used in the treatment or prevention of HIV or AIDS in a subject, e.g., in accordance with a method disclosed herein.

In another aspect, disclosed herein is a method of altering a cell, e.g., altering the structure, e.g., altering the sequence, of a target nucleic acid of a cell, comprising contacting said cell with: (a) a gRNA that targets the CCR5 gene, e.g., a gRNA as described herein; (b) a Cas9 molecule, e.g., a Cas9 molecule as described herein; and optionally, (c) a second, third and/or fourth gRNA that targets CCR5 gene, e.g., a second, third and/or fourth gRNA as described herein.

In an embodiment, the method comprises contacting said cell with (a) and (b).

In an embodiment, the method comprises contacting said cell with (a), (b), and (c).

The gRNA of (a) and optionally (c) may be selected from any of Tables 1A-1F, 2A-2C, 3A-3E, 4A-4C, 5A-5C, 6A-6E, 7A-7C, or 18, or a gRNA that differs by no more than 1, 2, 3, 4, or 5 nucleotides from, a targeting domain sequence from any of Tables 1A-1F, 2A-2C, 3A-3E, 4A-4C, 5A-5C, 6A-6E, 7A-7C, or 18.

In an embodiment, the method comprises contacting a cell from a subject suffering from or likely to develop an HIV infection or AIDS. The cell may be from a subject who does not have a mutation at a CCR5 target position.

In an embodiment, the cell being contacted in the disclosed method is a target cell from a circulating blood cell, a progenitor cell, or a stem cell, e.g., a hematopoietic stem cell (HSC) or a hematopoietic stem/progenitor cell (HSPC). In an embodiment, the target cell is a T cell (e.g., a CD4+ T cell, a CD8+ T cell, a helper T cell, a regulatory T cell, a cytotoxic T cell, a memory T cell, a T cell precursor or a natural killer T cell), a B cell (e.g., a progenitor B cell, a Pre B cell, a Pro B cell, a memory B cell, a plasma B cell), a monocyte, a megakaryocyte, a neutrophil, an eosinophil, a basophil, a mast cell, a reticulocyte, a lymphoid progenitor cell, a myeloid progenitor cell, or a hematopoietic stem cell. In an embodiment, the target cell is a bone marrow cell, (e.g., a lymphoid progenitor cell, a myeloid progenitor cell, an erythroid progenitor cell, a hematopoietic stem cell, or a mesenchymal stem cell). In an embodiment, the cell is a CD4 cell, a T cell, a gut associated lymphatic tissue (GALT), a macrophage, a dendritic cell, a myeloid precursor cell, or a microglia. The contacting may be performed ex vivo and the contacted cell may be returned to the subject's body after the contacting step. In another embodiment, the contacting step may be performed in vivo.

In an embodiment, the method of altering a cell as described herein comprises acquiring knowledge of the presence of a CCR5 target position in said cell, prior to the contacting step. Acquiring knowledge of the presence of a CCR5 target position in the cell may be by sequencing the CCR5 gene, or a portion of the CCR5 gene.

In an embodiment, the contacting step of the method comprises contacting the cell with a nucleic acid, e.g., a vector, e.g., an AAV vector, that expresses at least one of (a), (b), and (c). In an embodiment, the contacting step of the method comprises contacting the cell with a nucleic acid, e.g., a vector, e.g., an AAV vector, that encodes each of (a), (b), and (c). In another embodiment, the contacting step of the method comprises delivering to the cell a Cas9 molecule of (b) and a nucleic acid which encodes a gRNA of (a) and optionally, a second gRNA of (c)(i) (and further optionally, a third gRNA of (c)(ii) and/or fourth gRNA of (c)(iii).

In an embodiment, the contacting step comprises contacting the cell with a nucleic acid, e.g., a vector, e.g., an AAV vector, e.g., an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector. a described herein.

In an embodiment, the contacting step comprises delivering to the cell a Cas9 molecule of (b), as a protein or an mRNA, and a nucleic acid which encodes a gRNA of (a) and optionally a second, third and/or fourth gRNA of (c).

In an embodiment, the contacting step comprises delivering to the cell a Cas9 molecule of (b), as a protein or an mRNA, said gRNA of (a), as an RNA, and optionally said second, third and/or fourth gRNA of (c), as an RNA.

In an embodiment, the contacting step comprises delivering to the cell a gRNA of (a) as an RNA, optionally the second, third and/or fourth gRNA of (c) as an RNA, and a nucleic acid that encodes the Cas9 molecule of (b).

In an embodiment, the contacting step further comprises contacting the cell with an HSC self-renewal agonist, e.g., UM171 ((1r,4r)-N1-(2-benzyl-7-(2-methyl-2H-tetrazol-5-yl)-9H-pyrimido[4,5-b]indol-4-yl)cyclohexane-1,4-diamine) or a pyrimidoindole derivative described in Fares et al., Science, 2014, 345(6203): 1509-1512). In an embodiment, the cell is contacted with the HSC self-reneal agonist before (e.g., at least 1, 2, 4, 8, 12, 24, 36, or 48 hours before, e.g., about 2 hours before) the cell is contacted with a gRNA molecule and/or a Cas9 molecule. In another embodiment, the cell is contacted with the HSC self-reneal agonist after (e.g., at least 1, 2, 4, 8, 12, 24, 36, or 48 hours after, e.g., about 24 hours after) the cell is contacted with a gRNA molecule and/or a Cas9 molecule. In yet another embodiment, the cell is contacted with the HSC self-reneal agonist before (e.g., at least 1, 2, 4, 8, 12, 24, 36, or 48 hours before) and after (e.g., at least 1, 2, 4, 8, 12, 24, 36, or 48 hours after) the cell is contacted with a gRNA molecule and/or a Cas9 molecule. In an embodiment, the cell is contacted with the HSC self-reneal agonist about 2 hours before and about 24 hours after the cell is contacted with a gRNA molecule and/or a Cas9 molecule. In an embodiment, the cell is contacted with the HSC self-reneal agonist at the same time the cell is contacted with a gRNA molecule and/or a Cas9 molecule. In an embodiment, the HSC self-renewal agonist, e.g., UM171, is used at a concentration between 5 and 200 nM, e.g., between 10 and 100 nM or between 20 and 50 nM, e.g., about 40 nM.

In another aspect, disclosed herein is a cell or a population of cells produced (e.g., altered) by a method described herein.

In another aspect, disclosed herein is a method of treating a subject suffering from or likely to develop an HIV infection or AIDS, e.g., altering the structure, e.g., sequence, of a target nucleic acid of the subject, comprising contacting the subject (or a cell from the subject) with:

(a) a gRNA that targets the CCR5 gene, e.g., a gRNA disclosed herein;

(b) a Cas9 molecule, e.g., a Cas9 molecule disclosed herein; and

optionally, (c)(i) a second gRNA that targets the CCR5 gene, e.g., a second gRNA disclosed herein, and

further optionally, (c)(ii) a third gRNA, and still further optionally, (c)(iii) a fourth gRNA that target the CCR5 gene, e.g., a third and fourth gRNA disclosed herein.

In some embodiments, contacting comprises contacting with (a) and (b).

In some embodiments, contacting comprises contacting with (a), (b), and (c)(i). In some embodiments, contacting comprises contacting with (a), (b), (c)(i) and (c)(ii). In some embodiments, contacting comprises contacting with (a), (b), (c)(i), (c)(ii) and (c)(iii).

The gRNA of (a) or (c) (e.g., (c)(i), (c)(ii), or (c)(iii)) may be selected from any of Tables 1A-1F, 2A-2C, 3A-3E, 4A-4C, 5A-5C, 6A-6E, 7A-7C, or 18, or a gRNA that differs by no more than 1, 2, 3, 4, or 5 nucleotides from, a targeting domain sequence from any of Tables 1A-1F, 2A-2C, 3A-3E, 4A-4C, 5A-5C, 6A-6E, 7A-7C, or 18.

In an embodiment, the method comprises acquiring knowledge of the presence or absence of a mutation at a CCR5 target position in said subject.

In an embodiment, the method comprises acquiring knowledge of the presence or absence of a mutation at a CCR5 target position in said subject by sequencing the CCR5 gene or a portion of the CCR5 gene.

In an embodiment, the method comprises introducing a mutation at a CCR5 target position.

In an embodiment, the method comprises introducing a mutation at a CCR5 target position by NHEJ.

When the method comprises introducing a mutation at a CCR5 target position, e.g., by NHEJ in the coding region or a non-coding region, a Cas9 of (b) and at least one guide RNA (e.g., a guide RNA of (a)) are included in the contacting step.

In an embodiment, a cell of the subject is contacted ex vivo with (a), (b) and optionally (c)(i), further optionally (c)(ii), and still further optionally (c)(iii). In an embodiment, said cell is returned to the subject's body.

In an embodiment, a cell of the subject is contacted is in vivo with (a), (b) and optionally (c)(i), further optionally (c)(ii), and still further optionally (c)(iii). In an embodiment, the cell of the subject is contacted in vivo by intravenous delivery of (a), (b) and optionally (c)(i), further optionally (c)(ii), and still further optionally (c)(iii).

In an embodiment, the contacting step comprises contacting the subject with a nucleic acid, e.g., a vector, e.g., an AAV vector, described herein, e.g., a nucleic acid that encodes at least one of (a), (b), and optionally (c)(i), further optionally (c)(ii), and still further optionally (c)(iii).

In an embodiment, the contacting step comprises delivering to said subject said Cas9 molecule of (b), as a protein or mRNA, and a nucleic acid which encodes (a) and optionally (c)(i), further optionally (c)(ii), and still further optionally (c)(iii).

In an embodiment, the contacting step comprises delivering to the subject the Cas9 molecule of (b), as a protein or mRNA, said gRNA of (a), as an RNA, and optionally said second gRNA of (c)(i), further optionally said third gRNA of (c)(ii), and still further optionally said fourth gRNA of (c)(iii), as an RNA.

In an embodiment, the contacting step comprises delivering to the subject the gRNA of (a), as an RNA, optionally said second gRNA of (c)(i), further optionally said third gRNA of (c)(ii), and still further optionally said fourth gRNA of (c)(iii), as an RNA, and a nucleic acid that encodes the Cas9 molecule of (b).

In another aspect, disclosed herein is a reaction mixture comprising a gRNA molecule, a nucleic acid, or a composition described herein, and a cell, e.g., a cell from a subject having, or likely to develop and HIV infection or AIDS, or a subject having a mutation at a CCR5 target position (e.g., a heterozygous carrier of a CCR5 mutation).

In another aspect, disclosed herein is a kit comprising, (a) a gRNA molecule described herein, or a nucleic acid that encodes the gRNA, and one or more of the following:

(b) a Cas9 molecule, e.g., a Cas9 molecule described herein, or a nucleic acid or mRNA that encodes the Cas9;

(c)(i) a second gRNA molecule, e.g., a second gRNA molecule described herein or a nucleic acid that encodes (c)(i);

(c)(ii) a third gRNA molecule, e.g., a third gRNA molecule described herein or a nucleic acid that encodes (c)(ii);

(c)(iii) a fourth gRNA molecule, e.g., a fourth gRNA molecule described herein or a nucleic acid that encodes (c)(iii).

In an embodiment, the kit comprises a nucleic acid, e.g., an AAV vector, that encodes one or more of (a), (b), (c)(i), (c)(ii), and (c)(iii).

In yet another aspect, disclosed herein is a gRNA molecule, e.g., a gRNA molecule described herein, for use in treating, or delaying the onset or progression of, HIV infection or

AIDS in a subject, e.g., in accordance with a method of treating, or delaying the onset or progression of, HIV infection or AIDS as described herein.

In an embodiment, the gRNA molecule in used in combination with a Cas9 molecule, e.g., a Cas9 molecule described herein. Additionally or alternatively, in an embodiment, the gRNA molecule is used in combination with a second, third and/or fourth gRNA molecule, e.g., a second, third and/or fourth gRNA molecule described herein.

In still another aspect, disclosed herein is use of a gRNA molecule, e.g., a gRNA molecule described herein, in the manufacture of a medicament for treating, or delaying the onset or progression of, HIV infection or AIDS in a subject, e.g., in accordance with a method of treating, or delaying the onset or progression of, HIV infection or AIDS as described herein.

In an embodiment, the medicament comprises a Cas9 molecule, e.g., a Cas9 molecule described herein. Additionally or alternatively, in an embodiment, the medicament comprises a second, third and/or fourth gRNA molecule, e.g., a second, third and/or fourth gRNA molecule described herein.

The gRNA molecules and methods, as disclosed herein, can be used in combination with a governing gRNA molecule. As used herein, a governing gRNA molecule refers to a gRNA molecule comprising a targeting domain which is complementary to a target domain on a nucleic acid that encodes a component of the CRISPR/Cas system introduced into a cell or subject. For example, the methods described herein can further include contacting a cell or subject with a governing gRNA molecule or a nucleic acid encoding a governing molecule. In an embodiment, the governing gRNA molecule targets a nucleic acid that encodes a Cas9 molecule or a nucleic acid that encodes a target gene gRNA molecule. In an embodiment, the governing gRNA comprises a targeting domain that is complementary to a target domain in a sequence that encodes a Cas9 component, e.g., a Cas9 molecule or target gene gRNA molecule. In an embodiment, the target domain is designed with, or has, minimal homology to other nucleic acid sequences in the cell, e.g., to minimize off-target cleavage. For example, the targeting domain on the governing gRNA can be selected to reduce or minimize off-target effects. In an embodiment, a target domain for a governing gRNA can be disposed in the control or coding region of a Cas9 molecule or disposed between a control region and a transcribed region. In an embodiment, a target domain for a governing gRNA can be disposed in the control or coding region of a target gene gRNA molecule or disposed between a control region and a transcribed region for a target gene gRNA. While not wishing to be bound by theory, in an embodiment, it is believed that altering, e.g., inactivating, a nucleic acid that encodes a Cas9 molecule or a nucleic acid that encodes a target gene gRNA molecule can be effected by cleavage of the targeted nucleic acid sequence or by binding of a Cas9 molecule/governing gRNA molecule complex to the targeted nucleic acid sequence.

The compositions, reaction mixtures and kits, as disclosed herein, can also include a governing gRNA molecule, e.g., a governing gRNA molecule disclosed herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Headings, including numeric and alphabetical headings and subheadings, are for organization and presentation and are not intended to be limiting.

Other features and advantages of the invention will be apparent from the detailed description, drawings, and from the claims.

### BRIEF DESCRIPTION OF THE DRAWINGS

FIGS. 1A-1I are representations of several exemplary gRNAs.

FIG. 1A depicts a modular gRNA molecule derived in part (or modeled on a sequence in part) from Streptococcus pyogenes (S. pyogenes) as a duplexed structure (SEQ ID NOS: 42 and 43, respectively, in order of appearance);

FIG. 1B depicts a unimolecular (or chimeric) gRNA molecule derived in part from S. pyogenes as a duplexed structure (SEQ ID NO: 44);

FIG. 1C depicts a unimolecular gRNA molecule derived in part from S. pyogenes as a duplexed structure (SEQ ID NO: 45);

FIG. 1D depicts a unimolecular gRNA molecule derived in part from S. pyogenes as a duplexed structure (SEQ ID NO: 46);

FIG. 1E depicts a unimolecular gRNA molecule derived in part from S. pyogenes as a duplexed structure (SEQ ID NO: 47);

FIG. 1F depicts a modular gRNA molecule derived in part from Streptococcus thermophilus (S. thermophilus) as a duplexed structure (SEQ ID NOS: 48 and 49, respectively, in order of appearance);

FIG. 1G depicts an alignment of modular gRNA molecules of S. pyogenes and S. thermophilus (SEQ ID NOS: 50-53, respectively, in order of appearance).

FIGS. 1H-1I depicts additional exemplary structures of unimolecular gRNA molecules. FIG. 1H shows an exemplary structure of a unimolecular gRNA molecule derived in part from S. pyogenes as a duplexed structure (SEQ ID NO: 45). FIG. 1I shows an exemplary structure of a unimolecular gRNA molecule derived in part from S. aureus as a duplexed structure (SEQ ID NO: 40).

FIGS. 2A-2G depict an alignment of Cas9 sequences from Chylinski et al. (RNA Biol. 2013; 10(5): 726-737). The N-terminal RuvC-like domain is boxed and indicated with a “Y”. The other two RuvC-like domains are boxed and indicated with a “B”. The HNH-like domain is boxed and indicated by a “G”. Sm: S. mutans (SEQ ID NO: 1); Sp: S. pyogenes (SEQ ID NO: 2); St: S. thermophilus (SEQ ID NO: 3); Li: L. innocua (SEQ ID NO: 4). Motif: this is a motif based on the four sequences: residues conserved in all four sequences are indicated by single letter amino acid abbreviation; “*” indicates any amino acid found in the corresponding position of any of the four sequences; and “-” indicates any amino acid, e.g., any of the 20 naturally occurring amino acids, or absent.

FIGS. 3A-3B show an alignment of the N-terminal RuvC-like domain from the Cas9 molecules disclosed in Chylinski et at (SEQ ID NOS: 54-103, respectively, in order of appearance). The last line of FIG. 3B identifies 4 highly conserved residues.

FIGS. 4A-4B show an alignment of the N-terminal RuvC-like domain from the Cas9 molecules disclosed in Chylinski et al. with sequence outliers removed (SEQ ID NOS: 104-177, respectively, in order of appearance). The last line of FIG. 4B identifies 3 highly conserved residues.

FIGS. 5A-5C show an alignment of the HNH-like domain from the Cas9 molecules disclosed in Chylinski et at (SEQ ID NOS: 178-252, respectively, in order of appearance). The last line of FIG. 5C identifies conserved residues.

FIGS. 6A-6B show an alignment of the HNH-like domain from the Cas9 molecules disclosed in Chylinski et al. with sequence outliers removed (SEQ ID NOS: 253-302, respectively, in order of appearance). The last line of FIG. 6B identifies 3 highly conserved residues.

FIGS. 7A-7B depict an alignment of Cas9 sequences from S. pyogenes and Neisseria meningitidis (N. meningitidis). The N-terminal RuvC-like domain is boxed and indicated with a “Y”. The other two RuvC-like domains are boxed and indicated with a “B”. The HNH-like domain is boxed and indicated with a “G”. Sp: S. pyogenes; Nm: N. meningitidis. Motif: this is a motif based on the two sequences: residues conserved in both sequences are indicated by a single amino acid designation; “*” indicates any amino acid found in the corresponding position of any of the two sequences; “-” indicates any amino acid, e.g., any of the 20 naturally occurring amino acids, and “-” indicates any amino acid, e.g., any of the 20 naturally occurring amino acids, or absent.

FIG. 8 shows a nucleic acid sequence encoding Cas9 of N. meningitidis (SEQ ID NO: 303). Sequence indicated by an “R” is an SV40 NLS; sequence indicated as “G” is an HA tag; and sequence indicated by an “O” is a synthetic NLS sequence; the remaining (unmarked) sequence is the open reading frame (ORF).

FIGS. 9A-9B are schematic representations of the domain organization of S. pyogenes Cas 9. FIG. 9A shows the organization of the Cas9 domains, including amino acid positions, in reference to the two lobes of Cas9 (recognition (REC) and nuclease (NUC) lobes). FIG. 9B shows the percent homology of each domain across 83 Cas9 orthologs.

FIG. 10 depicts the efficiency of NHEJ mediated by a Cas9 molecule and exemplary gRNA molecules targeting the CCR5 locus.

FIG. 11 depicts flow cytometry analysis of genome edited HSCs to determine co-expression of stem cell phenotypic markers CD34 and CD90 and for viability (7-AAD-AnnexinV− cells). CD34+ HSCs maintain phenotype and viability after Nucleofection™ with Cas9 and CCR5 gRNA plasmid DNA (96 hours).

### DETAILED DESCRIPTION

### Definitions

“CCR5 target position”, as used herein, refers to any position that results in inactivation of the CCR5 gene. In an embodiment, a CCR5 target position refers to any of a CCR5 target knockout position or a CCR5 target knockdown position, as described herein.

“Domain”, as used herein, is used to describe segments of a protein or nucleic acid. Unless otherwise indicated, a domain is not required to have any specific functional property.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Governing gRNA molecule”, as used herein, refers to a gRNA molecule that comprises a targeting domain that is complementary to a target domain on a nucleic acid that comprises a sequence that encodes a component of the CRISPR/Cas system that is introduced into a cell or subject. A governing gRNA does not target an endogenous cell or subject sequence. In an embodiment, a governing gRNA molecule comprises a targeting domain that is complementary with a target sequence on: (a) a nucleic acid that encodes a Cas9 molecule; (b) a nucleic acid that encodes a gRNA which comprises a targeting domain that targets the CCR5 gene (a target gene gRNA); or on more than one nucleic acid that encodes a CRISPR/Cas component, e.g., both (a) and (b). In an embodiment, a nucleic acid molecule that encodes a CRISPR/Cas component, e.g., that encodes a Cas9 molecule or a target gene gRNA, comprises more than one target domain that is complementary with a governing gRNA targeting domain. While not wishing to be bound by theory, in an embodiment, it is believed that a governing gRNA molecule complexes with a Cas9 molecule and results in Cas9 mediated inactivation of the targeted nucleic acid, e.g., by cleavage or by binding to the nucleic acid, and results in cessation or reduction of the production of a CRISPR/Cas system component. In an embodiment, the Cas9 molecule forms two complexes: a complex comprising a Cas9 molecule with a target gene gRNA, which complex will alter the CCR5 gene; and a complex comprising a Cas9 molecule with a governing gRNA molecule, which complex will act to prevent further production of a CRISPR/Cas system component, e.g., a Cas9 molecule or a target gene gRNA molecule. In an embodiment, a governing gRNA molecule/Cas9 molecule complex binds to or promotes cleavage of a control region sequence, e.g., a promoter, operably linked to a sequence that encodes a Cas9 molecule, a sequence that encodes a transcribed region, an exon, or an intron, for the Cas9 molecule. In an embodiment, a governing gRNA molecule/Cas9 molecule complex binds to or promotes cleavage of a control region sequence, e.g., a promoter, operably linked to a gRNA molecule, or a sequence that encodes the gRNA molecule. In an embodiment, the governing gRNA, e.g., a Cas9-targeting governing gRNA molecule, or a target gene gRNA-targeting governing gRNA molecule, limits the effect of the Cas9 molecule/target gene gRNA molecule complex-mediated gene targeting. In an embodiment, a governing gRNA places temporal, level of expression, or other limits, on activity of the Cas9 molecule/target gene gRNA molecule complex. In an embodiment, a governing gRNA reduces off-target or other unwanted activity. In an embodiment, a governing gRNA molecule inhibits, e.g., entirely or substantially entirely inhibits, the production of a component of the Cas9 system and thereby limits, or governs, its activity.

“Modulator”, as used herein, refers to an entity, e.g., a drug, that can alter the activity (e.g., enzymatic activity, transcriptional activity, or translational activity), amount, distribution, or structure of a subject molecule or genetic sequence. In an embodiment, modulation comprises cleavage, e.g., breaking of a covalent or non-covalent bond, or the forming of a covalent or non-covalent bond, e.g., the attachment of a moiety, to the subject molecule. In an embodiment, a modulator alters the, three dimensional, secondary, tertiary, or quaternary structure, of a subject molecule. A modulator can increase, decrease, initiate, or eliminate a subject activity.

“Large molecule”, as used herein, refers to a molecule having a molecular weight of at least 2, 3, 5, 10, 20, 30, 40, 50, 60, 70, 80, 90, or 100 kD. Large molecules include proteins, polypeptides, nucleic acids, biologics, and carbohydrates.

“Polypeptide”, as used herein, refers to a polymer of amino acids having less than 100 amino acid residues. In an embodiment, it has less than 50, 20, or 10 amino acid residues.

“Reference molecule”, e.g., a reference Cas9 molecule or reference gRNA, as used herein, refers to a molecule to which a subject molecule, e.g., a subject Cas9 molecule of subject gRNA molecule, e.g., a modified or candidate Cas9 molecule is compared. For example, a Cas9 molecule can be characterized as having no more than 10% of the nuclease activity of a reference Cas9 molecule. Examples of reference Cas9 molecules include naturally occurring unmodified Cas9 molecules, e.g., a naturally occurring Cas9 molecule such as a Cas9 molecule of S. pyogenes, S. aureus or S. thermophilus. In an embodiment, the reference Cas9 molecule is the naturally occurring Cas9 molecule having the closest sequence identity or homology with the Cas9 molecule to which it is being compared. In an embodiment, the reference Cas9 molecule is a sequence, e.g., a naturally occurring or known sequence, which is the parental form on which a change, e.g., a mutation has been made.

“Replacement”, or “replaced”, as used herein with reference to a modification of a molecule does not require a process limitation but merely indicates that the replacement entity is present.

“Small molecule”, as used herein, refers to a compound having a molecular weight less than about 2 kD, e.g., less than about 2 kD, less than about 1.5 kD, less than about 1 kD, or less than about 0.75 kD.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human. In other embodiments, the subject is poultry.

“Treat”, “treating” and “treatment”, as used herein, mean the treatment of a disease in a mammal, e.g., in a human, including (a) inhibiting the disease, i.e., arresting or preventing its development; (b) relieving the disease, i.e., causing regression of the disease state; and (c) curing the disease.

“Prevent”, “preventing” and “prevention”, as used herein, means the prevention of a disease in a mammal, e.g., in a human, including (a) avoiding or precluding the disease; (2) affecting the predisposition toward the disease, e.g., preventing at least one symptom of the disease or to delay onset of at least one symptom of the disease.

“X” as used herein in the context of an amino acid sequence, refers to any amino acid (e.g., any of the twenty natural amino acids) unless otherwise specified.

### Human Immunodeficiency Virus

Human Immunodeficiency Virus (HIV) is a virus that causes severe immunodeficiency. In the United States, more than 1 million people are infected with the virus. Worldwide, approximately 30-40 million people are infected.

HIV is a single-stranded RNA virus that preferentially infects CD4 cells. The virus binds to receptors on the surface of CD4+ cells to enter and infect these cells. This binding and infection step is vital to the pathogenesis of HIV. The virus attaches to the CD4 receptor on the cell surface via its own surface glycoproteins, gp120 and gp41. These proteins are made from the cleavage product of gp160. Gp120 binds to a CD4 receptor and must also bind to another coreceptor in order for the virus to enter the host cell. In macrophage-(M-tropic) viruses, the coreceptor is CCR5 occasionally referred to as the CCR5 receptor. M-tropic virus is found most commonly in the early stages of HIV infection.

There are two types of HIV—HIV-1 and HIV-2. HIV-1 is the predominant global form and is a more virulent strain of the virus. HIV-2 has lower rates of infection and, at present, predominantly affects populations in West Africa. HIV is transmitted primarily through sexual exposure, although the sharing of needles in intravenous drug use is another mode of transmission.

As HIV infection progresses, the virus infects CD4 cells and a subject's CD4 counts fall. With declining CD4 counts, a subject is subject to increasing risk of opportunistic infections (OI). Severely declining CD4 counts are associated with a very high likelihood of OIs, specific cancers (such as Kaposi's sarcoma, Burkitt's lymphoma) and wasting syndrome. Normal CD4 counts are between 600-1200 cells/microliter.

Untreated HIV infection is a chronic, progressive disease that leads to acquired immunodeficiency syndrome (AIDS) and death in the vast majority of subjects. Diagnosis of AIDS is made based on infection with a variety of opportunistic pathogens, presence of certain cancers and/or CD4 counts below 200 cells/μL.

HIV was untreatable and invariably led to death until the late 1980's. Since then, antiretroviral therapy (ART) has dramatically slowed the course of HIV infection. Highly active antiretroviral therapy (HAART) is the use of three or more agents in combination to slow HIV. Antiretroviral therapy (ART) is indicated in a subject whose CD4 counts has dropped below 500 cells/μL. Viral load is the most common measurement of the efficacy of HIV treatment and disease progression. Viral load measures the amount of HIV RNA present in the blood.

Treatment with HAART has significantly altered the life expectancy of those infected with HIV. A subject in the developed world who maintains their HAART regimen can expect to live into their 60's and possibly 70's. However, HAART regimens are associated with significant, long term side effects. First, the dosing regimens are complex and associated with strict food requirements. Compliance rates with dosing can be lower than 50% in some populations in the United States. In addition, there are significant toxicities associated with HAART treatment, including diabetes, nausea, malaise, sleep disturbances. A subject who does not adhere to dosing requirements of HAART therapy may have return of viral load in their blood and are at risk for progression to disease and its associated complications.

### Methods to Treat or Prevent HIV Infection or AIDS

Methods and compositions described herein provide for a therapy, e.g., a one-time therapy, or a multi-dose therapy, that prevents or treats HIV infection and/or AIDS. In an embodiment, a disclosed therapy prevents, inhibits, or reduces the entry of HIV into CD4 cells of a subject who is already infected. While not wishing to be bound by theory, in an embodiment, it is believed that knocking out CCR5 on CD4 cells, renders the HIV virus unable to enter CD4 cells. Viral entry into CD4 cells requires interaction of the viral glycoproteins gp41 and gp120 with both the CD4 receptor and acoreceptor, e.g., CCR5. Once a functional coreceptor such as CCR5 has been eliminated from the surface of the CD4 cells, the virus is prevented from binding and entering the host CD4 cells. In an embodiment, the disease does not progress or has delayed progression compared to a subject who has not received the therapy.

While not wishing to be bound by theory, subjects with naturally occurring CCR5 receptor mutations who have delayed HIV progression may confer protection by the mechanism of action described herein. Subjects with a specific deletion in the CCR5 gene (e.g., the delta 32 deletion) have been shown to have much higher likelihood of being long-term non-progressors (meaning they did not require HAART and their HIV infection did not progress). See, e.g., Stewart G J et al., 1997 The Australian Long-Term Non-Progressor Study Group. Aids. 11:1833-1838. In addition, a subject who was CCR5+ (had a wild type CCR5 receptor) and infected with HIV underwent a bone marrow transplant for acute myeloid lymphoma. See, e.g., Hutter G et al., 2009N ENGL J MED. 360:692-698. The bone marrow transplant (BMT) was from a subject homozygous for a CCR5 delta 32 deletion. Following BMT, the subject did not have progression of HIV and did not require treatment with ART. These subjects offer evidence for the fact that introduction of a protective mutation of the CCR5 gene, or knockout or knockdown of the CCR5 gene prevents, delays or diminishes the ability of HIV to infect the subject. Mutation or deletion of the CCR5 gene, or reduced CCR5 gene expression, should therefore reduce the progression, virulence and pathology of HIV. In an embodiment, a method described herein is used to treat a subject having HIV.

In an embodiment, a method described herein is used to treat a subject having AIDS.

In an embodiment, a method described herein is used to prevent, or delay the onset or progression of, HIV infection and AIDS in a subject at high risk for HIV infection.

In an embodiment, a method described herein results in a selective advantage to survival of treated CD4 cells. Some proportion of CD4 cells will be modified and have a CCR5 protective mutation. These cells are not subject to infection with HIV. Cells that are not modified may be infected with HIV and are expected to undergo cell death. In an embodiment, after the treatment described herein, treated cells survive, while untreated cells die. This selective advantage drives eventual colonization in all body compartments with 100% CCR5-negative CD4 cells derived from treated cells, conferring complete protection in treated subjects against infection with M tropic HIV.

In an embodiment, the method comprises initiating treatment of a subject prior to disease onset.

In an embodiment, the method comprises initiating treatment of a subject after disease onset.

In an embodiment, the method comprises initiating treatment of a subject after disease onset, e.g., 1, 2, 3, 4, 5, 6, 7, 8, 9, 10, 12, 16, 24, 36, 48 or more months after onset of HIV infection or AIDS. While not wishing to be bound by theory, it is believed that this may be effective as disease progression is slow in some cases and a subject may present well into the course of illness.

In an embodiment, the method comprises initiating treatment of a subject in an advanced stage of disease, e.g., to slow viral replication and viral load.

Overall, initiation of treatment for a subject at all stages of disease is expected to prevent or reduce disease progression and benefit a subject.

In an embodiment, the method comprises initiating treatment of a subject prior to disease onset and prior to infection with HIV.

In an embodiment, the method comprises initiating treatment of a subject in an early stage of disease, e.g., when a subject has tested positive for HIV infection but has no signs or symptoms associated with HIV.

In an embodiment, the method comprises initiating treatment of a patient at the appearance of a reduced CD4 count or a positive HIV test.

In an embodiment, the method comprises treating a subject considered at risk for developing HIV infection.

In an embodiment, the method comprises treating a subject who is the spouse, partner, sexual partner, newborn, infant, or child of a subject with HIV.

In an embodiment, the method comprises treating a subject for the prevention or reduction of HIV infection.

In an embodiment, the method comprises treating a subject at the appearance of any of the following findings consistent with HIV: low CD4 count; opportunistic infections associated with HIV, including but not limited to: candidiasis, mycobacterium tuberculosis, cryptococcosis, cryptosporidiosis, cytomegalovirus; and/or malignancy associated with HIV, including but not limited to: lymphoma, Burkitt's lymphoma, or Kaposi's sarcoma.

In an embodiment, a cell is treated ex vivo and returned to a patient.

In an embodiment, an autologous CD4 cell can be treated ex vivo and returned to the subject.

In an embodiment, a heterologous CD4 cells can be treated ex vivo and transplanted into the subject.

In an embodiment, an autologous stem cell can be treated ex vivo and returned to the subject.

In an embodiment, a heterologous stem cell can be treated ex vivo and transplanted into the subject.

In an embodiment, the treatment comprises delivery of gRNA by intravenous injection, intramuscular injection; subcutaneous injection; intrathecal injection; or intraventricular injection.

In an embodiment, the treatment comprises delivery of a gRNA by an AAV.

In an embodiment, the treatment comprises delivery of a gRNA by a lentivirus.

In an embodiment, the treatment comprises delivery of a gRNA by a nanoparticle.

In an embodiment, the treatment comprises delivery of a gRNA by a parvovirus, e.g., a specifically a modified parvovirus designed to target bone marrow cells and/or CD4 cells.

In an embodiment, the treatment is initiated after a subject is determined to not have a mutation (e.g., an inactivating mutation, e.g., an inactivating mutation in either or both alleles) in CCR5 by genetic screening, e.g., genotyping, wherein the genetic testing was performed prior to or after disease onset.

### Methods of Targeting CCR5

As disclosed herein, the CCR5 gene can be targeted (e.g., altered) by gene editing, e.g., using CRISPR-Cas9 mediated methods as described herein.

Methods and compositions discussed herein, provide for targeting (e.g., altering) a CCR5 target position in the CCR5 gene. A CCR5 target position can be targeted (e.g., altered) by gene editing, e.g., using CRISPR-Cas9 mediated methods to target (e.g. alter) the CCR5 gene.

Disclosed herein are methods for targeting (e.g., altering) a CCR5 target position in the CCR5 gene. Targeting (e.g., altering) the CCR5 target position is achieved, e.g., by:

(1) knocking out the CCR5 gene:

(a) insertion or deletion (e.g., NHEJ-mediated insertion or deletion) of one or more nucleotides in close proximity to or within the early coding region of the CCR5 gene, or

(b) deletion (e.g., NHEJ-mediated deletion) of a genomic sequence including at least a portion of the CCR5 gene, or

(2) knocking down the CCR5 gene mediated by enzymatically inactive Cas9 (eiCas9) molecule or an eiCas9-fusion protein by targeting non-coding region, e.g., a promoter region, of the gene.

All approaches give rise to targeting (e.g., alteration) of the CCR5 gene.

In one embodiment, methods described herein introduce one or more breaks near the early coding region in at least one allele of the CCR5 gene. In another embodiment, methods described herein introduce two or more breaks to flank at least a portion of the CCR5 gene. The two or more breaks remove (e.g., delete) a genomic sequence including at least a portion of the CCR5 gene. In another embodiment, methods described herein comprise knocking down the CCR5 gene mediated by enzymatically inactive Cas9 (eiCas9) molecule or an eiCas9-fusion protein by targeting the promoter region of CCR5 target knockdown position. All methods described herein result in targeting (e.g., alteration) of the CCR5 gene.

The targeting (e.g., alteration) of the CCR5 gene can be mediated by any mechanism. Exemplary mechanisms that can be associated with the alteration of the CCR5 gene include, but are not limited to, non-homologous end joining (e.g., classical or alternative), microhomology-mediated end joining (MMEJ), homology-directed repair (e.g., endogenous donor template mediated), SDSA (synthesis dependent strand annealing), single strand annealing or single strand invasion.

### Knocking Out CCR5 by Introducing an Indel or a Deletion in the CCR5 Gene

In an embodiment, the method comprises introducing an insertion or deletion of one more nucleotides in close proximity to the CCR5 target knockout position (e.g., the early coding region) of the CCR5 gene. As described herein, in one embodiment, the method comprises the introduction of one or more breaks (e.g., single strand breaks or double strand breaks) sufficiently close to (e.g., either 5′ or 3′ to) the early coding region of the CCR5 target knockout position, such that the break-induced indel could be reasonably expected to span the CCR5 target knockout position (e.g., the early coding region). While not wishing to be bound by theory, it is believed that NHEJ-mediated repair of the break(s) allows for the NHEJ-mediated introduction of an indel in close proximity to within the early coding region of the CCR5 target knockout position.

In an embodiment, the method comprises introducing a deletion of a genomic sequence comprising at least a portion of the CCR5 gene. As described herein, in an embodiment, the method comprises the introduction of two double stand breaks—one 5′ and the other 3′ to (i.e., flanking) the CCR5 target position. In an embodiment, two gRNAs, e.g., unimolecular (or chimeric) or modular gRNA molecules, are configured to position the two double strand breaks on opposite sides of the CCR5 target knockout position in the CCR5 gene.

In an embodiment, a single strand break is introduced (e.g., positioned by one gRNA molecule) at or in close proximity to a CCR5 target position in the CCR5 gene. In an embodiment, a single gRNA molecule (e.g., with a Cas9 nickase) is used to create a single strand break at or in close proximity to the CCR5 target position, e.g., the gRNA is configured such that the single strand break is positioned either upstream (e.g., within 500 bp upstream, e.g., within 200 bp upstream) or downstream (e.g., within 500 bp downstream, e.g., within 200 bp downstream) of the CCR5 target position. In an embodiment, the break is positioned to avoid unwanted target chromosome elements, such as repeat elements, e.g., an Alu repeat.

In an embodiment, a double strand break is introduced (e.g., positioned by one gRNA molecule) at or in close proximity to a CCR5 target position in the CCR5 gene. In an embodiment, a single gRNA molecule (e.g., with a Cas9 nuclease other than a Cas9 nickase) is used to create a double strand break at or in close proximity to the CCR5 target position, e.g., the gRNA molecule is configured such that the double strand break is positioned either upstream (e.g., within 500 bp upstream, e.g., within 200 bp upstream) or downstream of (e.g., within 500 bp downstream, e.g., within 200 bp downstream) of a CCR5 target position. In an embodiment, the break is positioned to avoid unwanted target chromosome elements, such as repeat elements, e.g., an Alu repeat.

In an embodiment, two single strand breaks are introduced (e.g., positioned by two gRNA molecules) at or in close proximity to a CCR5 target position in the CCR5 gene. In an embodiment, two gRNA molecules (e.g., with one or two Cas9 nickases) are used to create two single strand breaks at or in close proximity to the CCR5 target position, e.g., the gRNAs molecules are configured such that both of the single strand breaks are positioned e.g., within 500 by upstream, e.g., within 200 bp upstream) or downstream (e.g., within 500 bp downstream, e.g., within 200 bp downstream) of the CCR5 target position. In another embodiment, two gRNA molecules (e.g., with two Cas9 nickases) are used to create two single strand breaks at or in close proximity to the CCR5 target position, e.g., the gRNAs molecules are configured such that one single strand break is positioned upstream (e.g., within 200 bp upstream) and a second single strand break is positioned downstream (e.g., within 200 bp downstream) of the CCR5 target position. In an embodiment, the breaks are positioned to avoid unwanted target chromosome elements, such as repeat elements, e.g., an Alu repeat.

In an embodiment, two double strand breaks are introduced (e.g., positioned by two gRNA molecules) at or in close proximity to a CCR5 target position in the CCR5 gene. In an embodiment, two gRNA molecules (e.g., with one or two Cas9 nucleases that are not Cas9 nickases) are used to create two double strand breaks to flank a CCR5 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CCR5 target position. In an embodiment, the breaks are positioned to avoid unwanted target chromosome elements, such as repeat elements, e.g., an Alu repeat.

In an embodiment, one double strand break and two single strand breaks are introduced (e.g., positioned by three gRNA molecules) at or in close proximity to a CCR5 target position in the CCR5 gene. In an embodiment, three gRNA molecules (e.g., with a Cas9 nuclease other than a Cas9 nickase and one or two Cas9 nickases) to create one double strand break and two single strand breaks to flank a CCR5 target position, e.g., the gRNA molecules are configured such that the double strand break is positioned upstream or downstream of (e.g., within 500 bp, e.g., within 200 bp upstream or downstream) of the CCR5 target position, and the two single strand breaks are positioned at the opposite site, e.g., downstream or upstream (e.g., within 500 bp, e.g., within 200 bp downstream or upstream), of the CCR5 target position. In an embodiment, the breaks are positioned to avoid unwanted target chromosome elements, such as repeat elements, e.g., an Alu repeat.

In an embodiment, four single strand breaks are introduced (e.g., positioned by four gRNA molecules) at or in close proximity to a CCR5 target position in the CCR5 gene. In an embodiment, four gRNA molecule (e.g., with one or more Cas9 nickases are used to create four single strand breaks to flank a CCR5 target position in the CCR5 gene, e.g., the gRNA molecules are configured such that a first and second single strand breaks are positioned upstream (e.g., within 500 bp upstream, e.g., within 200 bp upstream) of the CCR5 target position, and a third and a fourth single stranded breaks are positioned downstream (e.g., within 500 bp downstream, e.g., within 200 bp downstream) of the CCR5 target position. In an embodiment, the breaks are positioned to avoid unwanted target chromosome elements, such as repeat elements, e.g., an Alu repeat.

In an embodiment, two or more (e.g., three or four) gRNA molecules are used with one Cas9 molecule. In another embodiment, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

### Knocking Out CCR5 bp Deleting (e.g., NHEJ-Mediated Deletion) a Genomic Sequence Including at Least a Portion of the CCR5 Gene

In an embodiment, the method comprises deleting (e.g., NHEJ-mediated deletion) a genomic sequence including at least a portion of the CCR5 gene. As described herein, in one embodiment, the method comprises the introduction two sets of breaks (e.g., a pair of double strand breaks, one double strand break or a pair of single strand breaks, or two pairs of single strand breaks) to flank a region of the CCR5 gene (e.g., a coding region, e.g., an early coding region, or a non-coding region, e.g., a non-coding sequence of the CCR5 gene, e.g., a promoter, an enhancer, an intron, a 3′UTR, and/or a polyadenylation signal). While not wishing to be bound by theory, it is believed that NHEJ-mediated repair of the break(s) allows for alteration of the CCR5 gene as described herein, which reduces or eliminates expression of the gene, e.g., to knock out one or both alleles of the CCR5 gene.

In an embodiment, two double strand breaks are introduced (e.g., positioned by two gRNA molecules) at or in close proximity to a CCR5 target position in the CCR5 gene. In an embodiment, two gRNA molecules (e.g., with one or two Cas9 nucleases that are not Cas9 nickases) are used to create two double strand breaks to flank a CCR5 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CCR5 target position. In an embodiment, the breaks are positioned to avoid unwanted target chromosome elements, such as repeat elements, e.g., an Alu repeat.

In an embodiment, one double strand break and two single strand breaks are introduced (e.g., positioned by three gRNA molecules) at or in close proximity to a CCR5 target position in the CCR5 gene. In an embodiment, three gRNA molecules (e.g., with a Cas9 nuclease other than a Cas9 nickase and one or two Cas9 nickases) to create one double strand break and two single strand breaks to flank a CCR5 target position, e.g., the gRNA molecules are configured such that the double strand break is positioned upstream or downstream of (e.g., within 500 bp, e.g., within 200 bp upstream or downstream) of the CCR5 target position, and the two single strand breaks are positioned at the opposite site, e.g., downstream or upstream (e.g., within 500 bp, e.g., within 200 bp downstream or upstream), of the CCR5 target position. In an embodiment, the breaks are positioned to avoid unwanted target chromosome elements, such as repeat elements, e.g., an Alu repeat.

In an embodiment, four single strand breaks are introduced (e.g., positioned by four gRNA molecules) at or in close proximity to a CCR5 target position in the CCR5 gene. In an embodiment, four gRNA molecule (e.g., with one or more Cas9 nickases are used to create four single strand breaks to flank a CCR5 target position in the CCR5 gene, e.g., the gRNA molecules are configured such that a first and second single strand breaks are positioned upstream (e.g., within 500 bp upstream, e.g., within 200 bp upstream) of the CCR5 target position, and a third and a fourth single stranded breaks are positioned downstream (e.g., within 500 bp downstream, e.g., within 200 bp downstream) of the CCR5 target position. In an embodiment, the breaks are positioned to avoid unwanted target chromosome elements, such as repeat elements, e.g., an Alu repeat.

In an embodiment, two or more (e.g., three or four) gRNA molecules are used with one Cas9 molecule. In another embodiment, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

Knocking Down CCR5 Mediated by an Enzymatically Inactive Cas9 (eiCas9) Molecule

A targeted knockdown approach reduces or eliminates expression of functional CCR5 gene product. As described herein, in an embodiment, a targeted knockdown is mediated by targeting an enzymatically inactive Cas9 (eiCas9) molecule or an eiCas9 fused to a transcription repressor domain or chromatin modifying protein to alter transcription, e.g., to block, reduce, or decrease transcription, of the CCR5 gene.

Methods and compositions discussed herein may be used to alter the expression of the CCR5 gene to treat or prevent HIV infection or AIDS by targeting a promoter region of the CCR5 gene. In an embodiment, the promoter region is targeted to knock down expression of the CCR5 gene. A targeted knockdown approach reduces or eliminates expression of functional CCR5 gene product. As described herein, in an embodiment, a targeted knockdown is mediated by targeting an enzymatically inactive Cas9 (eiCas9) or an eiCas9 fused to a transcription repressor domain or chromatin modifying protein to alter transcription, e.g., to block, reduce, or decrease transcription, of the CCR5 gene.

In an embodiment, one or more eiCas9s may be used to block binding of one or more endogenous transcription factors. In another embodiment, an eiCas9 can be fused to a chromatin modifying protein. Altering chromatin status can result in decreased expression of the target gene. One or more eiCas9s fused to one or more chromatin modifying proteins may be used to alter chromatin status.

I. gRNA Molecules

A gRNA molecule, as that term is used herein, refers to a nucleic acid that promotes the specific targeting or homing of a gRNA molecule/Cas9 molecule complex to a target nucleic acid. gRNA molecules can be unimolecular (having a single RNA molecule), sometimes referred to herein as “chimeric” gRNAs, or modular (comprising more than one, and typically two, separate RNA molecules). A gRNA molecule comprises a number of domains. The gRNA molecule domains are described in more detail below.

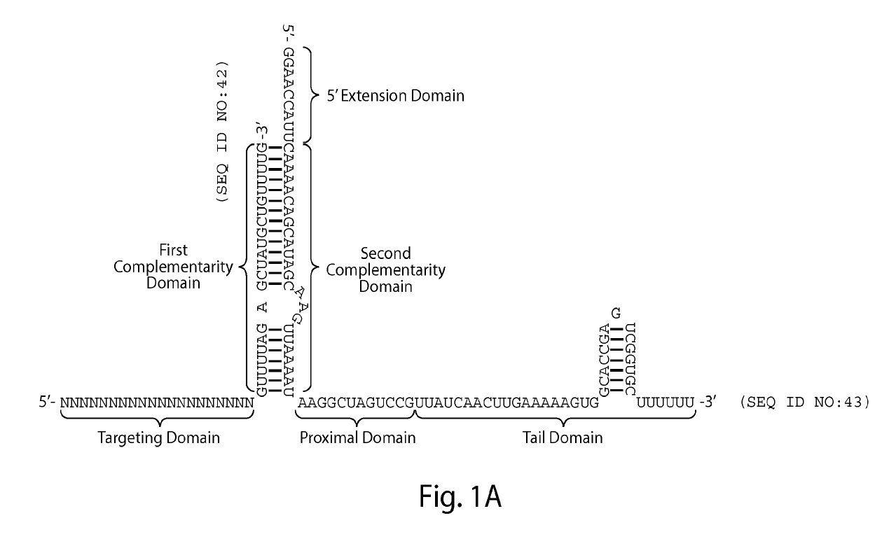

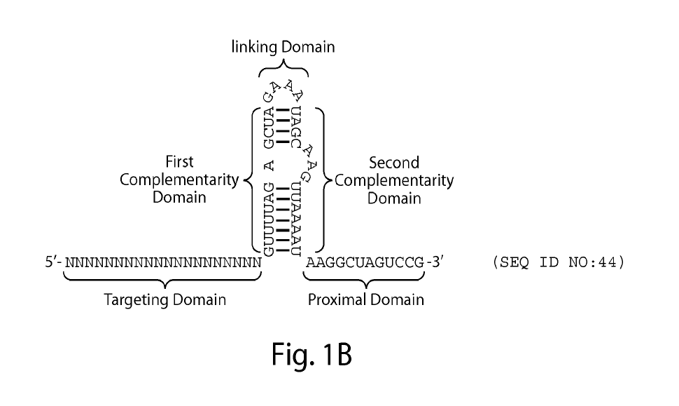

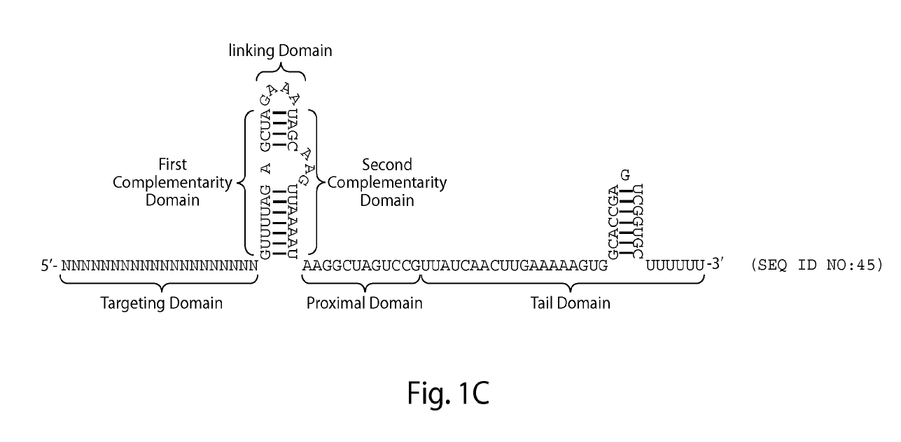

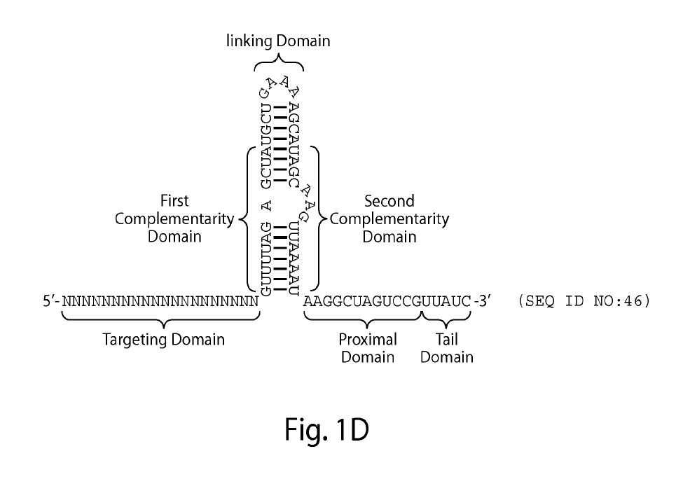

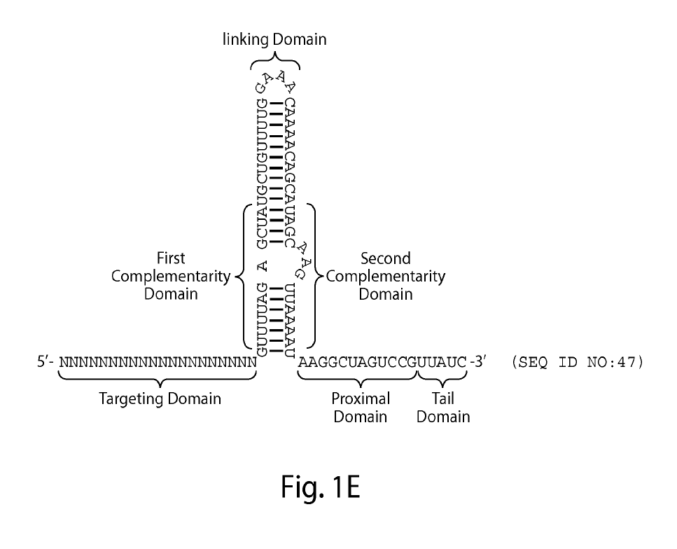

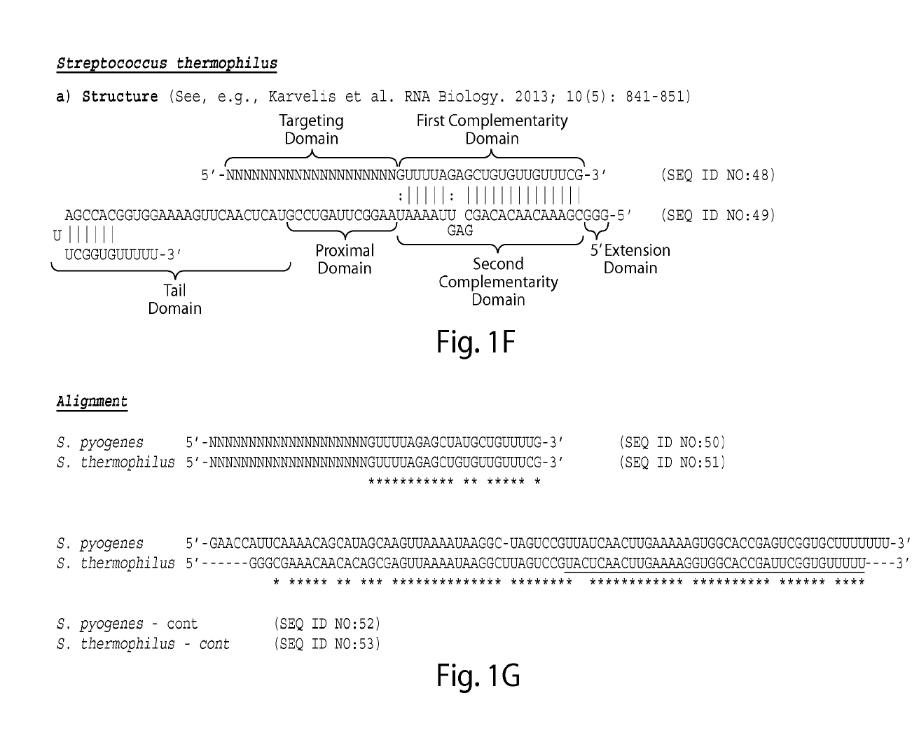

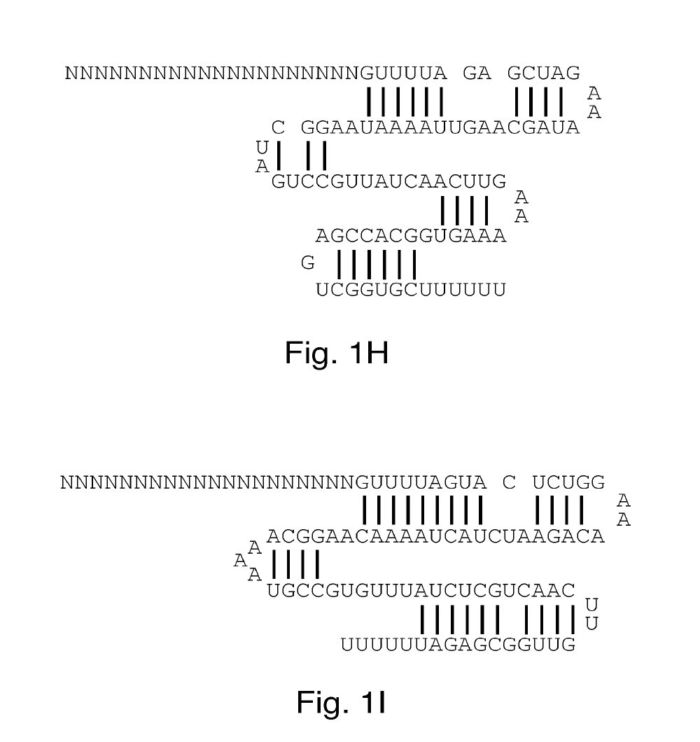

Several exemplary gRNA structures, with domains indicated thereon, are provided in FIG. 1. While not wishing to be bound by theory, in an embodiment, with regard to the three dimensional form, or intra- or inter-strand interactions of an active form of a gRNA, regions of high complementarity are sometimes shown as duplexes in FIGS. 1A-1G and other depictions provided herein.

In an embodiment, a unimolecular, or chimeric, gRNA comprises, preferably from 5′ to 3′:

a targeting domain (which is complementary to a target nucleic acid in the CCR5 gene, e.g., a targeting domain from any of Tables 1A-1F);

a first complementarity domain;

a linking domain;

a second complementarity domain (which is complementary to the first complementarity domain);

a proximal domain; and

optionally, a tail domain.

In an embodiment, a modular gRNA comprises:
a first strand comprising, preferably from 5′ to 3′;
   a targeting domain (which is complementary to a target nucleic acid in the CCR5 gene, e.g., a targeting domain from Tables 1A-1F); and
   a first complementarity domain; and
a second strand, comprising, preferably from 5′ to 3′:
   optionally, a 5′ extension domain;
   a second complementarity domain;
   a proximal domain; and
   optionally, a tail domain.

The domains are discussed briefly below:

The Targeting Domain

FIGS. 1A-1G provide examples of the placement of targeting domains.

The targeting domain comprises a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The strand of the target nucleic acid with which the targeting domain is complementary is referred to herein as the complementary strand. Some or all of the nucleotides of the domain can have a modification, e.g., a modification found in Section VIII herein.

In an embodiment, the targeting domain is 16 nucleotides in length.

In an embodiment, the targeting domain is 17 nucleotides in length.

In an embodiment, the targeting domain is 18 nucleotides in length.

In an embodiment, the targeting domain is 19 nucleotides in length.

In an embodiment, the targeting domain is 20 nucleotides in length.

In an embodiment, the targeting domain is 21 nucleotides in length.

In an embodiment, the targeting domain is 22 nucleotides in length.

In an embodiment, the targeting domain is 23 nucleotides in length.

In an embodiment, the targeting domain is 24 nucleotides in length.

In an embodiment, the targeting domain is 25 nucleotides in length.

In an embodiment, the targeting domain is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

Targeting domains are discussed in more detail below.

The First Complementarity Domain

FIGS. 1A-1G provide examples of first complementarity domains.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain is 5 to 25 nucleotides in length. In an embodiment, the first complementary domain is 7 to 25 nucleotides in length. In an embodiment, the first complementary domain is 7 to 22 nucleotides in length. In an embodiment, the first complementary domain is 7 to 18 nucleotides in length. In an embodiment, the first complementary domain is 7 to 15 nucleotides in length. In an embodiment, the first complementary domain is 5, 6, 7, 8, 9, 10, 11, 12, 13, 14, 15, 16, 17, 18, 19, 20, 21, 22, 23, 24, or 25 nucleotides in length.

In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4-9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length.

The first complementarity domain can share homology with, or be derived from, a naturally occurring first complementarity domain. In an embodiment, it has at least 50% homology with a first complementarity domain disclosed herein, e.g., an S. pyogenes, S. aureus or S. thermophilus, first complementarity domain.

Some or all of the nucleotides of the domain can have a modification, e.g., modification found in Section VIII herein.

First complementarity domains are discussed in more detail below.

The Linking Domain

FIGS. 1A-1G provide examples of linking domains.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain covalently couples the first and second complementarity domains, see, e.g., FIGS. 1B-1E. In an embodiment, the linking domain is, or comprises, a covalent bond interposed between the first complementarity domain and the second complementarity domain. Typically the linking domain comprises one or more, e.g., 2, 3, 4, 5, 6, 7, 8, 9, or 10 nucleotides.

In modular gRNA molecules the two molecules are associated by virtue of the hybridization of the complementarity domains see e.g., FIG. 1A.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length. In an embodiment, a linking domain is 2, 3, 4, 5, 6, 7, 8, 9, 10, 15, 20, or 25 or more nucleotides in length. In an embodiment, a linking domain is 2 to 50, 2 to 40, 2 to 30, 2 to 20, 2 to 10, or 2 to 5 nucleotides in length. In an embodiment, a linking domain shares homology with, or is derived from, a naturally occurring sequence, e.g., the sequence of a tracrRNA that is 5′ to the second complementarity domain. In an embodiment, the linking domain has at least 50% homology with a linking domain disclosed herein.

Some or all of the nucleotides of the domain can have a modification, e.g., modification found in Section VIII herein.

Linking domains are discussed in more detail below.

The 5′ Extension Domain

In an embodiment, a modular gRNA can comprise additional sequence, 5′ to the second complementarity domain, referred to herein as the 5′ extension domain, see, e.g., FIG. 1A. In an embodiment, the 5′ extension domain is, 2 to 10, 2 to 9, 2 to 8, 2 to 7, 2 to 6, 2 to 5, or 2 to 4 nucleotides in length. In an embodiment, the 5′ extension domain is 2, 3, 4, 5, 6, 7, 8, 9, or 10 or more nucleotides in length.

The Second Complementarity Domain

FIGS. 1A-1G provide examples of second complementarity domains.

The second complementarity domain is complementary with the first complementarity domain, and in an embodiment, has sufficient complementarity to the second complementarity domain to form a duplexed region under at least some physiological conditions. In an embodiment, e.g., as shown in FIGS. 1A-1B, the second complementarity domain can include sequence that lacks complementarity with the first complementarity domain, e.g., sequence that loops out from the duplexed region.

In an embodiment, the second complementarity domain is 5 to 27 nucleotides in length. In an embodiment, it is longer than the first complementarity region. In an embodiment the second complementary domain is 7 to 27 nucleotides in length. In an embodiment, the second complementary domain is 7 to 25 nucleotides in length. In an embodiment, the second complementary domain is 7 to 20 nucleotides in length. In an embodiment, the second complementary domain is 7 to 17 nucleotides in length. In an embodiment, the complementary domain is 5, 6, 7, 8, 9, 10, 11, 12, 13, 14, 15, 16, 17, 18, 19, 20, 21, 22, 23, 24 or 25 nucleotides in length.

In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length.

In an embodiment, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In an embodiment, it has at least 50% homology with a second complementarity domain disclosed herein, e.g., an S. pyogenes, S. aureus or S. thermophilus, first complementarity domain.

Some or all of the nucleotides of the domain can have a modification, e.g., modification found in Section VIII herein.

A Proximal Domain

FIGS. 1A-1G provide examples of proximal domains.

In an embodiment, the proximal domain is 5 to 20 nucleotides in length. In an embodiment, the proximal domain can share homology with or be derived from a naturally occurring proximal domain. In an embodiment, it has at least 50% homology with a proximal domain disclosed herein, e.g., an S. pyogenes, S. aureus or S. thermophilus, proximal domain.

Some or all of the nucleotides of the domain can have a modification, e.g., modification found in Section VIII herein.

A Tail Domain

FIGS. 1A-1G provide examples of tail domains.

As can be seen by inspection of the tail domains in FIGS. 1A-1E, a broad spectrum of tail domains are suitable for use in gRNA molecules. In an embodiment, the tail domain is 0 (absent), 1, 2, 3, 4, 5, 6, 7, 8, 9, or 10 nucleotides in length. In embodiment, the tail domain nucleotides are from or share homology with sequence from the 5′ end of a naturally occurring tail domain, see e.g., FIG. 1D or FIG. 1E. In an embodiment, the tail domain includes sequences that are complementary to each other and which, under at least some physiological conditions, form a duplexed region.

In an embodiment, the tail domain is absent or is 1 to 50 nucleotides in length. In an embodiment, the tail domain can share homology with or be derived from a naturally occurring proximal tail domain. In an embodiment, it has at least 50% homology with a tail domain disclosed herein, e.g., an S. pyogenes, S. aureus or S. thermophilus, tail domain.

In an embodiment, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

The domains of gRNA molecules are described in more detail below.

The Targeting Domain

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011).

In an embodiment, the targeting domain is 16, 17, 18, 19, 20, 21, 22, 23, 24, 25 or 26 nucleotides in length.

In an embodiment, the targeting domain is 16 nucleotides in length.

In an embodiment, the targeting domain is 17 nucleotides in length.

In an embodiment, the targeting domain is 18 nucleotides in length.

In an embodiment, the targeting domain is 19 nucleotides in length.

In an embodiment, the targeting domain is 20 nucleotides in length.

In an embodiment, the targeting domain is 21 nucleotides in length.

In an embodiment, the targeting domain is 22 nucleotides in length.

In an embodiment, the targeting domain is 23 nucleotides in length.

In an embodiment, the targeting domain is 24 nucleotides in length.

In an embodiment, the targeting domain is 25 nucleotides in length.

In an embodiment, the targeting domain is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

In an embodiment, the targeting domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the targeting domain is 20+/−5 nucleotides in length.

In an embodiment, the targeting domain is 20+/−10, 30+/−10, 40+/−10, 50+/−10, 60+/−10, 70+/−10, 80+/−10, 90+/−10, or 100+/−10 nucleotides, in length.

In an embodiment, the targeting domain is 30+/−10 nucleotides in length.

In an embodiment, the targeting domain is 10 to 100, 10 to 90, 10 to 80, 10 to 70, 10 to 60, 10 to 50, 10 to 40, 10 to 30, 10 to 20 or 10 to 15 nucleotides in length. In another embodiment, the targeting domain is 20 to 100, 20 to 90, 20 to 80, 20 to 70, 20 to 60, 20 to 50, 20 to 40, 20 to 30, or 20 to 25 nucleotides in length.

Typically the targeting domain has full complementarity with the target sequence. In an embodiment, the targeting domain has or includes 1, 2, 3, 4, 5, 6, 7 or 8 nucleotides that are not complementary with the corresponding nucleotide of the targeting domain.

In an embodiment, the target domain includes 1, 2, 3, 4 or 5 nucleotides that are complementary with the corresponding nucleotide of the targeting domain within 5 nucleotides of its 5′ end. In an embodiment, the target domain includes 1, 2, 3, 4 or 5 nucleotides that are complementary with the corresponding nucleotide of the targeting domain within 5 nucleotides of its 3′ end.

In an embodiment, the target domain includes 1, 2, 3, or 4 nucleotides that are not complementary with the corresponding nucleotide of the targeting domain within 5 nucleotides of its 5′ end. In an embodiment, the target domain includes 1, 2, 3, or 4 nucleotides that are not complementary with the corresponding nucleotide of the targeting domain within 5 nucleotides of its 3′ end.

In an embodiment, the degree of complementarity, together with other properties of the gRNA, is sufficient to allow targeting of a Cas9 molecule to the target nucleic acid.

In some embodiments, the targeting domain comprises two consecutive nucleotides that are not complementary to the target domain (“non-complementary nucleotides”), e.g., two consecutive noncomplementary nucleotides that are within 5 nucleotides of the 5′ end of the targeting domain, within 5 nucleotides of the 3′ end of the targeting domain, or more than 5 nucleotides away from one or both ends of the targeting domain.

In an embodiment, no two consecutive nucleotides within 5 nucleotides of the 5′ end of the targeting domain, within 5 nucleotides of the 3′ end of the targeting domain, or within a region that is more than 5 nucleotides away from one or both ends of the targeting domain, are not complementary to the targeting domain.

In an embodiment, there are no noncomplementary nucleotides within 5 nucleotides of the 5′ end of the targeting domain, within 5 nucleotides of the 3′ end of the targeting domain, or within a region that is more than 5 nucleotides away from one or both ends of the targeting domain.

In an embodiment, the targeting domain nucleotides do not comprise modifications, e.g., modifications of the type provided in Section VIII. However, in an embodiment, the targeting domain comprises one or more modifications, e.g., modifications that it render it less susceptible to degradation or more bio-compatible, e.g., less immunogenic. By way of example, the backbone of the targeting domain can be modified with a phosphorothioate, or other modification(s) from Section VIII. In an embodiment, a nucleotide of the targeting domain can comprise a 2′ modification, e.g., a 2-acetylation, e.g., a 2′ methylation, or other modification(s) from Section VIII.

In some embodiments, the targeting domain includes 1, 2, 3, 4, 5, 6, 7 or 8 or more modifications. In an embodiment, the targeting domain includes 1, 2, 3, or 4 modifications within 5 nucleotides of its 5′ end. In an embodiment, the targeting domain comprises as many as 1, 2, 3, or 4 modifications within 5 nucleotides of its 3′ end.

In some embodiments, the targeting domain comprises modifications at two consecutive nucleotides, e.g., two consecutive nucleotides that are within 5 nucleotides of the 5′ end of the targeting domain, within 5 nucleotides of the 3′ end of the targeting domain, or more than 5 nucleotides away from one or both ends of the targeting domain.

In an embodiment, no two consecutive nucleotides are modified within 5 nucleotides of the 5′ end of the targeting domain, within 5 nucleotides of the 3′ end of the targeting domain, or within a region that is more than 5 nucleotides away from one or both ends of the targeting domain. In an embodiment, no nucleotide is modified within 5 nucleotides of the 5′ end of the targeting domain, within 5 nucleotides of the 3′ end of the targeting domain, or within a region that is more than 5 nucleotides away from one or both ends of the targeting domain.

Modifications in the targeting domain can be selected to not interfere with targeting efficacy, which can be evaluated by testing a candidate modification in the system described in Section IV. gRNAs having a candidate targeting domain having a selected length, sequence, degree of complementarity, or degree of modification, can be evaluated in a system in Section IV. The candidate targeting domain can be placed, either alone, or with one or more other candidate changes in a gRNA molecule/Cas9 molecule system known to be functional with a selected target and evaluated.

In an embodiment, all of the modified nucleotides are complementary to and capable of hybridizing to corresponding nucleotides present in the target domain. In another embodiment, 1, 2, 3, 4, 5, 6, 7 or 8 or more modified nucleotides are not complementary to or capable of hybridizing to corresponding nucleotides present in the target domain.

In an embodiment, the targeting domain comprises, preferably in the 5′→3′ direction: a secondary domain and a core domain. These domains are discussed in more detail below.

The Core Domain and Secondary Domain of the Targeting Domain

The “core domain” of the targeting domain is complementary to the “core domain target” on the target nucleic acid. In an embodiment, the core domain comprises about 8 to about 13 nucleotides from the 3′ end of the targeting domain (e.g., the most 3′ 8 to 13 nucleotides of the targeting domain).

In an embodiment, the core domain and targeting domain, are independently, 6+/−2, 7+/−2, 8+/−2, 9+/−2, 10+/−2, 11+/−2, 12+/−2, 13+/−2, 14+/−2, 15+/−2, or 16+-2, 17+/−2, or 18+/−2, nucleotides in length.

In an embodiment, the core domain and targeting domain, are independently 10+/−2 nucleotides in length.

In an embodiment, the core domain and targeting domain, are independently, 10+/−4 nucleotides in length.

In an embodiment, the core domain and targeting domain are independently 6, 7, 8, 9, 10, 11, 12, 13, 14, 15, 16, 17, or 18, nucleotides in length.

In an embodiment, the core domain and targeting domain are independently 3 to 20, 4 to 20, 5 to 20, 6 to 20, 7 to 20, 8 to 20, 9 to 20 10 to 20 or 15 to 20 nucleotides in length.

In an embodiment, the core domain and targeting domain are independently 3 to 15, e.g., 6 to 15, 7 to 14, 7 to 13, 6 to 12, 7 to 12, 7 to 11, 7 to 10, 8 to 14, 8 to 13, 8 to 12, 8 to 11, 8 to 10 or 8 to 9 nucleotides in length.

The “core domain” is complementary with the “core domain target” of the target nucleic acid. Typically the core domain has exact complementarity with the core domain target. In some embodiments, the core domain can have 1, 2, 3, 4 or 5 nucleotides that are not complementary with the corresponding nucleotide of the core domain. In an embodiment, the degree of complementarity, together with other properties of the gRNA, is sufficient to allow targeting of a Cas9 molecule to the target nucleic acid.

The “secondary domain” of the targeting domain of the gRNA is complementary to the “secondary domain target” of the target nucleic acid.

In an embodiment, the secondary domain is positioned 5′ to the core domain.

In an embodiment, the secondary domain is absent or optional.

In an embodiment, if the targeting domain is 26 nucleotides in length and the core domain (counted from the 3′ end of the targeting domain) is 8 to 13 nucleotides in length, the secondary domain is 12 to 17 nucleotides in length.

In an embodiment, if the targeting domain is 25 nucleotides in length and the core domain (counted from the 3′ end of the targeting domain) is 8 to 13 nucleotides in length, the secondary domain is 12 to 17 nucleotides in length.

In an embodiment, if the targeting domain is 24 nucleotides in length and the core domain (counted from the 3′ end of the targeting domain) is 8 to 13 nucleotides in length, the secondary domain is 11 to 16 nucleotides in length.

In an embodiment, if the targeting domain is 23 nucleotides in length and the core domain (counted from the 3′ end of the targeting domain) is 8 to 13 nucleotides in length, the secondary domain is 10 to 15 nucleotides in length.

In an embodiment, if the targeting domain is 22 nucleotides in length and the core domain (counted from the 3′ end of the targeting domain) is 8 to 13 nucleotides in length, the secondary domain is 9 to 14 nucleotides in length.

In an embodiment, if the targeting domain is 21 nucleotides in length and the core domain (counted from the 3′ end of the targeting domain) is 8 to 13 nucleotides in length, the secondary domain is 8 to 13 nucleotides in length.

In an embodiment, if the targeting domain is 20 nucleotides in length and the core domain (counted from the 3′ end of the targeting domain) is 8 to 13 nucleotides in length, the secondary domain is 7 to 12 nucleotides in length.

In an embodiment, if the targeting domain is 19 nucleotides in length and the core domain (counted from the 3′ end of the targeting domain) is 8 to 13 nucleotides in length, the secondary domain is 6 to 11 nucleotides in length.

In an embodiment, if the targeting domain is 18 nucleotides in length and the core domain (counted from the 3′ end of the targeting domain) is 8 to 13 nucleotides in length, the secondary domain is 5 to 10 nucleotides in length.

In an embodiment, if the targeting domain is 17 nucleotides in length and the core domain (counted from the 3′ end of the targeting domain) is 8 to 13 nucleotides in length, the secondary domain is 4 to 9 nucleotides in length.

In an embodiment, if the targeting domain is 16 nucleotides in length and the core domain (counted from the 3′ end of the targeting domain) is 8 to 13 nucleotides in length, the secondary domain is 3 to 8 nucleotides in length.

In an embodiment, the secondary domain is 0, 1, 2, 3, 4, 5, 6, 7, 8, 9, 10, 11, 12, 13, 14 or 15 nucleotides in length.

The secondary domain is complementary with the secondary domain target. Typically the secondary domain has exact complementarity with the secondary domain target. In some embodiments the secondary domain can have 1, 2, 3, 4 or 5 nucleotides that are not complementary with the corresponding nucleotide of the secondary domain. In an embodiment, the degree of complementarity, together with other properties of the gRNA, is sufficient to allow targeting of a Cas9 molecule to the target nucleic acid.

In an embodiment, the core domain nucleotides do not comprise modifications, e.g., modifications of the type provided in Section VIII. However, in an embodiment, the core domain comprises one or more modifications, e.g., modifications that it render it less susceptible to degradation or more bio-compatible, e.g., less immunogenic. By way of example, the backbone of the core domain can be modified with a phosphorothioate, or other modification(s) from Section VIII. In an embodiment a nucleotide of the core domain can comprise a 2′ modification, e.g., a 2-acetylation, e.g., a 2′ methylation, or other modification(s) from Section VIII. Typically, a core domain will contain no more than 1, 2, or 3 modifications.

Modifications in the core domain can be selected to not interfere with targeting efficacy, which can be evaluated by testing a candidate modification in the system described in Section IV. gRNAs having a candidate core domain having a selected length, sequence, degree of complementarity, or degree of modification, can be evaluated in the system described at Section IV. The candidate core domain can be placed, either alone, or with one or more other candidate changes in a gRNA molecule/Cas9 molecule system known to be functional with a selected target and evaluated.

In an embodiment, the secondary domain nucleotides do not comprise modifications, e.g., modifications of the type provided in Section VIII. However, in an embodiment, the secondary domain comprises one or more modifications, e.g., modifications that render it less susceptible to degradation or more bio-compatible, e.g., less immunogenic. By way of example, the backbone of the secondary domain can be modified with a phosphorothioate, or other modification(s) from Section VIII. In an embodiment a nucleotide of the secondary domain can comprise a 2′ modification, e.g., a 2-acetylation, e.g., a 2′ methylation, or other modification(s) from Section VIII. Typically, a secondary domain will contain no more than 1, 2, or 3 modifications.

Modifications in the secondary domain can be selected to not interfere with targeting efficacy, which can be evaluated by testing a candidate modification in the system described in Section IV. gRNAs having a candidate secondary domain having a selected length, sequence, degree of complementarity, or degree of modification, can be evaluated in the system described at Section IV. The candidate secondary domain can be placed, either alone, or with one or more other candidate changes in a gRNA molecule/Cas9 molecule system known to be functional with a selected target and evaluated.

In an embodiment, (1) the degree of complementarity between the core domain and its target, and (2) the degree of complementarity between the secondary domain and its target, may differ. In an embodiment, (1) may be greater than (2). In an embodiment, (1) may be less than (2). In an embodiment, (1) and (2) are the same, e.g., each may be completely complementary with its target.

In an embodiment, (1) the number of modifications (e.g., modifications from Section VIII) of the nucleotides of the core domain and (2) the number of modification (e.g., modifications from Section VIII) of the nucleotides of the secondary domain, may differ. In an embodiment, (1) may be less than (2). In an embodiment, (1) may be greater than (2). In an embodiment, (1) and (2) may be the same, e.g., each may be free of modifications.

The First and Second Complementarity Domains

The first complementarity domain is complementary with the second complementarity domain.

Typically the first domain does not have exact complementarity with the second complementarity domain target. In some embodiments, the first complementarity domain can have 1, 2, 3, 4 or 5 nucleotides that are not complementary with the corresponding nucleotide of the second complementarity domain. In an embodiment, 1, 2, 3, 4, 5 or 6, e.g., 3 nucleotides, will not pair in the duplex, and, e.g., form a non-duplexed or looped-out region. In an embodiment, an unpaired, or loop-out, region, e.g., a loop-out of 3 nucleotides, is present on the second complementarity domain. In an embodiment, the unpaired region begins 1, 2, 3, 4, 5, or 6, e.g., 4, nucleotides from the 5′ end of the second complementarity domain.

In an embodiment, the degree of complementarity, together with other properties of the gRNA, is sufficient to allow targeting of a Cas9 molecule to the target nucleic acid.

In an embodiment, the first and second complementarity domains are:

independently, 6+/−2, 7+/−2, 8+/−2, 9+/−2, 10+/−2, 11+/−2, 12+/−2, 13+/−2, 14+/−2, 15+/−2, 16+/−2, 17+/−2, 18+/−2, 19+/−2, or 20+/−2, 21+/−2, 22+/−2, 23+/−2, or 24+/−2 nucleotides in length;

independently, 6, 7, 8, 9, 10, 11, 12, 13, 14, 14, 16, 17, 18, 19, 20, 21, 22, 23, 24, 25, or 26, nucleotides in length; or

independently, 5 to 24, 5 to 23, 5 to 22, 5 to 21, 5 to 20, 7 to 18, 9 to 16, or 10 to 14 nucleotides in length.

In an embodiment, the second complementarity domain is longer than the first complementarity domain, e.g., 2, 3, 4, 5, or 6, e.g., 6, nucleotides longer.

In an embodiment, the first and second complementary domains, independently, do not comprise modifications, e.g., modifications of the type provided in Section VIII.

In an embodiment, the first and second complementary domains, independently, comprise one or more modifications, e.g., modifications that the render the domain less susceptible to degradation or more bio-compatible, e.g., less immunogenic. By way of example, the backbone of the domain can be modified with a phosphorothioate, or other modification(s) from Section VIII. In an embodiment, a nucleotide of the domain can comprise a 2′ modification, e.g., a 2-acetylation, e.g., a 2′ methylation, or other modification(s) from Section VIII.

In an embodiment, the first and second complementary domains, independently, include 1, 2, 3, 4, 5, 6, 7 or 8 or more modifications. In an embodiment, the first and second complementary domains, independently, include 1, 2, 3, or 4 modifications within 5 nucleotides of its 5′ end. In an embodiment, the first and second complementary domains, independently, include as many as 1, 2, 3, or 4 modifications within 5 nucleotides of its 3′ end.

In an embodiment, the first and second complementary domains, independently, include modifications at two consecutive nucleotides, e.g., two consecutive nucleotides that are within 5 nucleotides of the 5′ end of the domain, within 5 nucleotides of the 3′ end of the domain, or more than 5 nucleotides away from one or both ends of the domain. In an embodiment, the first and second complementary domains, independently, include no two consecutive nucleotides that are modified, within 5 nucleotides of the 5′ end of the domain, within 5 nucleotides of the 3′ end of the domain, or within a region that is more than 5 nucleotides away from one or both ends of the domain. In an embodiment, the first and second complementary domains, independently, include no nucleotide that is modified within 5 nucleotides of the 5′ end of the domain, within 5 nucleotides of the 3′ end of the domain, or within a region that is more than 5 nucleotides away from one or both ends of the domain.

Modifications in a complementarity domain can be selected to not interfere with targeting efficacy, which can be evaluated by testing a candidate modification in the system described in Section IV. gRNAs having a candidate complementarity domain having a selected length, sequence, degree of complementarity, or degree of modification, can be evaluated in the system described in Section IV. The candidate complementarity domain can be placed, either alone, or with one or more other candidate changes in a gRNA molecule/Cas9 molecule system known to be functional with a selected target and evaluated.

In an embodiment, the first complementarity domain has at least 60, 70, 80, 85%, 90% or 95% homology with, or differs by no more than 1, 2, 3, 4, 5, or 6 nucleotides from, a reference first complementarity domain, e.g., a naturally occurring, e.g., an S. pyogenes, S. aureus or S. thermophilus, first complementarity domain, or a first complementarity domain described herein, e.g., from FIGS. 1A-1G.

In an embodiment, the second complementarity domain has at least 60, 70, 80, 85%, 90%, or 95% homology with, or differs by no more than 1, 2, 3, 4, 5, or 6 nucleotides from, a reference second complementarity domain, e.g., a naturally occurring, e.g., an S. pyogenes, S. aureus or S. thermophilus, second complementarity domain, or a second complementarity domain described herein, e.g., from FIGS. 1A-1G.

The duplexed region formed by first and second complementarity domains is typically 6, 7, 8, 9, 10, 11, 12, 13, 14, 15, 16, 17, 18, 19, 20, 21 or 22 base pairs in length (excluding any looped out or unpaired nucleotides).

In some embodiments, the first and second complementarity domains, when duplexed, comprise 11 paired nucleotides, for example, in the gRNA sequence (one paired strand underlined, one bolded):

(SEQ ID NO: 5)

NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGC.

In some embodiments, the first and second complementarity domains, when duplexed, comprise 15 paired nucleotides, for example in the gRNA sequence (one paired strand underlined, one bolded):

(SEQ ID NO: 27)

NNNNNNNNNNNNNNNNNNNNGUUUUAGAGCUAUGCUGAAAAGCAUAGCAA
GUUAAAAUAAGGCUAGUCCGUUAUCAACUUGAAAAAGUGGCACCGAGUCG
GUGC.

In some embodiments the first and second complementarity domains, when duplexed, comprise 16 paired nucleotides, for example in the gRNA sequence (one paired strand underlined, one bolded):

(SEQ ID NO: 28)

NNNNNNNNNNNNNNNNNNNNGUUUUAGAGCUAUGCUGGAAACAGCAUAGC
AAGUUAAAAUAAGGCUAGUCCGUUAUCAACUUGAAAAAGUGGCACCGAG
UCGGUGC.

In some embodiments the first and second complementarity domains, when duplexed, comprise 21 paired nucleotides, for example in the gRNA sequence (one paired strand underlined, one bolded):

(SEQ ID NO: 29)

NNNNNNNNNNNNNNNNNNNNGUUUUAGAGCUAUGCUGUUUUGGAAACAAA
ACAGCAUAGCAAGUUAAAAUAAGGCUAGUCCGUUAUCAACUUGAAAAAGU
GGCACCGAGUCGGUGC.

In some embodiments, nucleotides are exchanged to remove poly-U tracts, for example in the gRNA sequences (exchanged nucleotides underlined):

(SEQ ID NO: 30)

NNNNNNNNNNNNNNNNNNNNGUAUUAGAGCUAGAAAUAGCAAGUUAAUAU
AAGGCUAGUCCGUUAUCAACUUGAAAAAGUGGCACCGAGUCGGUGC;

(SEQ ID NO: 31)

NNNNNNNNNNNNNNNNNNNNGUUUAAGAGCUAGAAAUAGCAAGUUUAAAU
AAGGCUAGUCCGUUAUCAACUUGAAAAAGUGGCACCGAGUCGGUGC;
or

(SEQ ID NO: 32)

NNNNNNNNNNNNNNNNNNNNGUAUUAGAGCUAUGCUGUAUUGGAAACAAU
ACAGCAUAGCAAGUUAAUAUAAGGCUAGUCCGUUAUCAACUUGAAAAAGU
GGCACCGAGUCGGUGC.

### The 5′ Extension Domain

In an embodiment, a modular gRNA can comprise additional sequence, 5′ to the second complementarity domain. In an embodiment, the 5′ extension domain is 2 to 10, 2 to 9, 2 to 8, 2 to 7, 2 to 6, 2 to 5, or 2 to 4 nucleotides in length. In an embodiment, the 5′ extension domain is 2, 3, 4, 5, 6, 7, 8, 9, or 10 or more nucleotides in length.

In an embodiment, the 5′ extension domain nucleotides do not comprise modifications, e.g., modifications of the type provided in Section VIII. However, in an embodiment, the 5′ extension domain comprises one or more modifications, e.g., modifications that it render it less susceptible to degradation or more bio-compatible, e.g., less immunogenic. By way of example, the backbone of the 5′ extension domain can be modified with a phosphorothioate, or other modification(s) from Section VIII. In an embodiment, a nucleotide of the 5′ extension domain can comprise a 2′ modification, e.g., a 2-acetylation, e.g., a 2′ methylation, or other modification(s) from Section VIII.

In some embodiments, the 5′ extension domain can comprise as many as 1, 2, 3, 4, 5, 6, 7 or 8 modifications. In an embodiment, the 5′ extension domain comprises as many as 1, 2, 3, or 4 modifications within 5 nucleotides of its 5′ end, e.g., in a modular gRNA molecule. In an embodiment, the 5′ extension domain comprises as many as 1, 2, 3, or 4 modifications within 5 nucleotides of its 3′ end, e.g., in a modular gRNA molecule.

In some embodiments, the 5′ extension domain comprises modifications at two consecutive nucleotides, e.g., two consecutive nucleotides that are within 5 nucleotides of the 5′ end of the 5′ extension domain, within 5 nucleotides of the 3′ end of the 5′ extension domain, or more than 5 nucleotides away from one or both ends of the 5′ extension domain. In an embodiment, no two consecutive nucleotides are modified within 5 nucleotides of the 5′ end of the 5′ extension domain, within 5 nucleotides of the 3′ end of the 5′ extension domain, or within a region that is more than 5 nucleotides away from one or both ends of the 5′ extension domain. In an embodiment, no nucleotide is modified within 5 nucleotides of the 5′ end of the 5′ extension domain, within 5 nucleotides of the 3′ end of the 5′ extension domain, or within a region that is more than 5 nucleotides away from one or both ends of the 5′ extension domain.

Modifications in the 5′ extension domain can be selected to not interfere with gRNA molecule efficacy, which can be evaluated by testing a candidate modification in the system described in Section IV. gRNAs having a candidate 5′ extension domain having a selected length, sequence, degree of complementarity, or degree of modification, can be evaluated in the system described at Section IV. The candidate 5′ extension domain can be placed, either alone, or with one or more other candidate changes in a gRNA molecule/Cas9 molecule system known to be functional with a selected target and evaluated.

In an embodiment, the 5′ extension domain has at least 60, 70, 80, 85, 90 or 95% homology with, or differs by no more than 1, 2, 3, 4, 5, or 6 nucleotides from, a reference 5′ extension domain, e.g., a naturally occurring, e.g., an S. pyogenes, S. aureus or S. thermophilus, 5′ extension domain, or a 5′ extension domain described herein, e.g., from FIGS. 1A-1G.

### The Linking Domain

In a unimolecular gRNA molecule the linking domain is disposed between the first and second complementarity domains. In a modular gRNA molecule, the two molecules are associated with one another by the complementarity domains.

In an embodiment, the linking domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the linking domain is 20+/−10, 30+/−10, 40+/−10, 50+/−10, 60+/−10, 70+/−10, 80+/−10, 90+/−10, or 100+/−10 nucleotides, in length.

In an embodiment, the linking domain is 10 to 100, 10 to 90, 10 to 80, 10 to 70, 10 to 60, 10 to 50, 10 to 40, 10 to 30, 10 to 20 or 10 to 15 nucleotides in length. In other embodiments, the linking domain is 20 to 100, 20 to 90, 20 to 80, 20 to 70, 20 to 60, 20 to 50, 20 to 40, 20 to 30, or 20 to 25 nucleotides in length.

In an embodiment, the linking domain is 1, 2, 3, 4, 5, 6, 7, 8, 9, 10, 11, 12, 13, 14, 15, 16 17, 18, 19, or 20 nucleotides in length.

In and embodiment, the linking domain is a covalent bond.

In an embodiment, the linking domain comprises a duplexed region, typically adjacent to or within 1, 2, or 3 nucleotides of the 3′ end of the first complementarity domain and/or the 5-end of the second complementarity domain. In an embodiment, the duplexed region can be 20+/−10 base pairs in length. In an embodiment, the duplexed region can be 10+/−5, 15+/−5, 20+/−5, or 30+/−5 base pairs in length. In an embodiment, the duplexed region can be 1, 2, 3, 4, 5, 6, 7, 8, 9, 10, 11, 12, 13, 14, or 15 base pairs in length.

Typically the sequences forming the duplexed region have exact complementarity with one another, though in some embodiments as many as 1, 2, 3, 4, 5, 6, 7 or 8 nucleotides are not complementary with the corresponding nucleotides.

In an embodiment, the linking domain nucleotides do not comprise modifications, e.g., modifications of the type provided in Section VIII. However, in an embodiment, the linking domain comprises one or more modifications, e.g., modifications that it render it less susceptible to degradation or more bio-compatible, e.g., less immunogenic. By way of example, the backbone of the linking domain can be modified with a phosphorothioate, or other modification(s) from Section VIII. In an embodiment a nucleotide of the linking domain can comprise a 2′ modification, e.g., a 2-acetylation, e.g., a 2′ methylation, or other modification(s) from Section VIII.

In some embodiments, the linking domain can comprise as many as 1, 2, 3, 4, 5, 6, 7 or 8 modifications.

Modifications in a linking domain can be selected to not interfere with targeting efficacy, which can be evaluated by testing a candidate modification in the system described in Section IV. gRNAs having a candidate linking domain having a selected length, sequence, degree of complementarity, or degree of modification, can be evaluated a system described in Section IV. A candidate linking domain can be placed, either alone, or with one or more other candidate changes in a gRNA molecule/Cas9 molecule system known to be functional with a selected target and evaluated.

In an embodiment, the linking domain has at least 60, 70, 80, 85, 90 or 95% homology with, or differs by no more than 1, 2, 3, 4, 5, or 6 nucleotides from, a reference linking domain, e.g., a linking domain described herein, e.g., from FIGS. 1A-1G.

The Proximal Domain

In an embodiment, the proximal domain is 6+/−2, 7+/−2, 8+/−2, 9+/−2, 10+/−2, 11+/−2, 12+/−2, 13+/−2, 14+/−2, 14+/−2, 16+/−2, 17+/−2, 18+/−2, 19+/−2, or 20+/−2 nucleotides in length.

In an embodiment, the proximal domain is 6, 7, 8, 9, 10, 11, 12, 13, 14, 15, 16, 17, 18, 19, 20, 21, 22, 23, 24, 25 or 26 nucleotides in length.

In an embodiment, the proximal domain is 5 to 20, 7, to 18, 9 to 16, or 10 to 14 nucleotides in length.

In an embodiment, the proximal domain nucleotides do not comprise modifications, e.g., modifications of the type provided in Section VIII. However, in an embodiment, the proximal domain comprises one or more modifications, e.g., modifications that it render it less susceptible to degradation or more bio-compatible, e.g., less immunogenic. By way of example, the backbone of the proximal domain can be modified with a phosphorothioate, or other modification(s) from Section VIII. In an embodiment a nucleotide of the proximal domain can comprise a 2′ modification, e.g., a 2-acetylation, e.g., a 2′ methylation, or other modification(s) from Section VIII.

In some embodiments, the proximal domain can comprise as many as 1, 2, 3, 4, 5, 6, 7 or 8 modifications. In an embodiment, the proximal domain comprises as many as 1, 2, 3, or 4 modifications within 5 nucleotides of its 5′ end, e.g., in a modular gRNA molecule. In an embodiment, the target domain comprises as many as 1, 2, 3, or 4 modifications within 5 nucleotides of its 3′ end, e.g., in a modular gRNA molecule.

In some embodiments, the proximal domain comprises modifications at two consecutive nucleotides, e.g., two consecutive nucleotides that are within 5 nucleotides of the 5′ end of the proximal domain, within 5 nucleotides of the 3′ end of the proximal domain, or more than 5 nucleotides away from one or both ends of the proximal domain. In an embodiment, no two consecutive nucleotides are modified within 5 nucleotides of the 5′ end of the proximal domain, within 5 nucleotides of the 3′ end of the proximal domain, or within a region that is more than 5 nucleotides away from one or both ends of the proximal domain. In an embodiment, no nucleotide is modified within 5 nucleotides of the 5′ end of the proximal domain, within 5 nucleotides of the 3′ end of the proximal domain, or within a region that is more than 5 nucleotides away from one or both ends of the proximal domain.

Modifications in the proximal domain can be selected so as to not interfere with gRNA molecule efficacy, which can be evaluated by testing a candidate modification in the system described in Section IV. gRNAs having a candidate proximal domain having a selected length, sequence, degree of complementarity, or degree of modification, can be evaluated in the system described at Section IV. The candidate proximal domain can be placed, either alone, or with one or more other candidate changes in a gRNA molecule/Cas9 molecule system known to be functional with a selected target and evaluated.

In an embodiment, the proximal domain has at least 60, 70, 80, 85 90 or 95% homology with, or differs by no more than 1, 2, 3, 4, 5, or 6 nucleotides from, a reference proximal domain, e.g., a naturally occurring, e.g., an S. pyogenes, S. aureus or S. thermophilus, proximal domain, or a proximal domain described herein, e.g., from FIGS. 1A-1G.

The Tail Domain

In an embodiment, the tail domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the tail domain is 20+/−5 nucleotides in length.

In an embodiment, the tail domain is 20+/−10, 30+/−10, 40+/−10, 50+/−10, 60+/−10, 70+/−10, 80+/−10, 90+/−10, or 100+/−10 nucleotides, in length.

In an embodiment, the tail domain is 25+/−10 nucleotides in length.

In an embodiment, the tail domain is 10 to 100, 10 to 90, 10 to 80, 10 to 70, 10 to 60, 10 to 50, 10 to 40, 10 to 30, 10 to 20 or 10 to 15 nucleotides in length.

In other embodiments, the tail domain is 20 to 100, 20 to 90, 20 to 80, 20 to 70, 20 to 60, 20 to 50, 20 to 40, 20 to 30, or 20 to 25 nucleotides in length.

In an embodiment, the tail domain is 1 to 20, 1 to 15, 1 to 10, or 1 to 5 nucleotides in length.

In an embodiment, the tail domain nucleotides do not comprise modifications, e.g., modifications of the type provided in Section VIII. However, in an embodiment, the tail domain comprises one or more modifications, e.g., modifications that it render it less susceptible to degradation or more bio-compatible, e.g., less immunogenic. By way of example, the backbone of the tail domain can be modified with a phosphorothioate, or other modification(s) from Section VIII. In an embodiment a nucleotide of the tail domain can comprise a 2′ modification, e.g., a 2-acetylation, e.g., a 2′ methylation, or other modification(s) from Section VIII.

In some embodiments, the tail domain can have as many as 1, 2, 3, 4, 5, 6, 7 or 8 modifications. In an embodiment, the target domain comprises as many as 1, 2, 3, or 4 modifications within 5 nucleotides of its 5′ end. In an embodiment, the target domain comprises as many as 1, 2, 3, or 4 modifications within 5 nucleotides of its 3′ end.

In an embodiment, the tail domain comprises a tail duplex domain, which can form a tail duplexed region. In an embodiment, the tail duplexed region can be 3, 4, 5, 6, 7, 8, 9, 10, 11, or 12 base pairs in length. In an embodiment, a further single stranded domain, exists 3′ to the tail duplexed domain. In an embodiment, this domain is 3, 4, 5, 6, 7, 8, 9, or 10 nucleotides in length. In an embodiment it is 4 to 6 nucleotides in length.

In an embodiment, the tail domain has at least 60, 70, 80, or 90% homology with, or differs by no more than 1, 2, 3, 4, 5, or 6 nucleotides from, a reference tail domain, e.g., a naturally occurring, e.g., an S. pyogenes, S. aureus or S. thermophilus, tail domain, or a tail domain described herein, e.g., from FIGS. 1A-1G.

In an embodiment, the proximal and tail domain, taken together comprise the following sequences:

(SEQ ID NO: 33)

AAGGCUAGUCCGUUAUCAACUUGAAAAAGUGGCACCGAGUCGGUGCU,
or

(SEQ ID NO: 34)

AAGGCUAGUCCGUUAUCAACUUGAAAAAGUGGCACCGAGUCGGUGGUGC,
or

(SEQ ID NO: 35)

AAGGCUAGUCCGUUAUCAACUUGAAAAAGUGGCACCGAGUCGGUGCGGAU
C,
or

(SEQ ID NO: 36)

AAGGCUAGUCCGUUAUCAACUUGAAAAAGUG,
or

(SEQ ID NO: 37)

AAGGCUAGUCCGUUAUCA,
or

(SEQ ID NO: 38)

AAGGCUAGUCCG.

In an embodiment, the tail domain comprises the 3′ sequence UUUUUU, e.g., if a U6 promoter is used for transcription.

In an embodiment, the tail domain comprises the 3′ sequence UUUU, e.g., if an H1 promoter is used for transcription.

In an embodiment, tail domain comprises variable numbers of 3′ Us depending, e.g., on the termination signal of the pol-III promoter used.

In an embodiment, the tail domain comprises variable 3′ sequence derived from the DNA template if a T7 promoter is used.

In an embodiment, the tail domain comprises variable 3′ sequence derived from the DNA template, e.g., if in vitro transcription is used to generate the RNA molecule.

In an embodiment, the tail domain comprises variable 3′ sequence derived from the DNA template, e.g., if a pol-II promoter is used to drive transcription.

Modifications in the tail domain can be selected to not interfere with targeting efficacy, which can be evaluated by testing a candidate modification in the system described in Section IV. gRNAs having a candidate tail domain having a selected length, sequence, degree of complementarity, or degree of modification, can be evaluated in the system described in Section IV. The candidate tail domain can be placed, either alone, or with one or more other candidate changes in a gRNA molecule/Cas9 molecule system known to be functional with a selected target and evaluated.

In an embodiment, the tail domain comprises modifications at two consecutive nucleotides, e.g., two consecutive nucleotides that are within 5 nucleotides of the 5′ end of the tail domain, within 5 nucleotides of the 3′ end of the tail domain, or more than 5 nucleotides away from one or both ends of the tail domain. In an embodiment, no two consecutive nucleotides are modified within 5 nucleotides of the 5′ end of the tail domain, within 5 nucleotides of the 3′ end of the tail domain, or within a region that is more than 5 nucleotides away from one or both ends of the tail domain. In an embodiment, no nucleotide is modified within 5 nucleotides of the 5′ end of the tail domain, within 5 nucleotides of the 3′ end of the tail domain, or within a region that is more than 5 nucleotides away from one or both ends of the tail domain.

In an embodiment, a gRNA has the following structure:

5′ [targeting domain]-[first complementarity domain]-[linking domain]-[second complementarity domain]-[proximal domain]-[tail domain]-3′

wherein, the targeting domain comprises a core domain and optionally a secondary domain, and is 10 to 50 nucleotides in length;

the first complementarity domain is 5 to 25 nucleotides in length and, in an embodiment, has at least 50, 60, 70, 80, 85, 90 or 95% homology with a reference first complementarity domain disclosed herein;

the linking domain is 1 to 5 nucleotides in length;

the second complementarity domain is 5 to 27 nucleotides in length and, in an embodiment has at least 50, 60, 70, 80, 85, 90 or 95% homology with a reference second complementarity domain disclosed herein;

the proximal domain is 5 to 20 nucleotides in length and, in an embodiment, has at least 50, 60, 70, 80, 85, 90 or 95% homology with a reference proximal domain disclosed herein; and

the tail domain is absent or a nucleotide sequence is 1 to 50 nucleotides in length and, in an embodiment, has at least 50, 60, 70, 80, 85, 90 or 95% homology with a reference tail domain disclosed herein.

Exemplary Chimeric gRNAs

In an embodiment, a unimolecular, or chimeric, gRNA comprises, preferably from 5′ to 3′:

a targeting domain (which is complementary to a target nucleic acid);

a first complementarity domain, e.g., comprising 15, 16, 17, 18, 19, 20, 21, 22, 23, 24, 25, or 26 nucleotides;

a linking domain;

a second complementarity domain (which is complementary to the first complementarity domain);

a proximal domain; and

a tail domain, wherein,

(a) the proximal and tail domain, when taken together, comprise

at least 15, 18, 20, 25, 30, 31, 35, 40, 45, 49, 50, or 53 nucleotides;

(b) there are at least 15, 18, 20, 25, 30, 31, 35, 40, 45, 49, 50, or 53 nucleotides 3′ to the last nucleotide of the second complementarity domain; or

(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein.

In an embodiment, the proximal and tail domain, when taken together, comprise at least 15, 18, 20, 25, 30, 31, 35, 40, 45, 49, 50, or 53 nucleotides.

In an embodiment, there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length.

In an embodiment, the targeting domain comprises, has, or consists of, 17 nucleotides (e.g., 17 consecutive nucleotides) having complementarity with the target domain, e.g., the targeting domain is 17 nucleotides in length.

In an embodiment, the targeting domain comprises, has, or consists of, 18 nucleotides (e.g., 18 consecutive nucleotides) having complementarity with the target domain, e.g., the targeting domain is 18 nucleotides in length.

In an embodiment, the targeting domain comprises, has, or consists of, 19 nucleotides (e.g., 19 consecutive nucleotides) having complementarity with the target domain, e.g., the targeting domain is 19 nucleotides in length.

In an embodiment, the targeting domain comprises, has, or consists of, 20 nucleotides (e.g., 20 consecutive nucleotides) having complementarity with the target domain, e.g., the targeting domain is 20 nucleotides in length.

In an embodiment, the targeting domain comprises, has, or consists of, 21 nucleotides (e.g., 21 consecutive nucleotides) having complementarity with the target domain, e.g., the targeting domain is 21 nucleotides in length.

In an embodiment, the targeting domain comprises, has, or consists of, 22 nucleotides (e.g., 22 consecutive nucleotides) having complementarity with the target domain, e.g., the targeting domain is 22 nucleotides in length.

In an embodiment, the targeting domain comprises, has, or consists of, 23 nucleotides (e.g., 23 consecutive nucleotides) having complementarity with the target domain, e.g., the targeting domain is 23 nucleotides in length.

In an embodiment, the targeting domain comprises, has, or consists of, 24 nucleotides (e.g., 24 consecutive nucleotides) having complementarity with the target domain, e.g., the targeting domain is 24 nucleotides in length.

In an embodiment, the targeting domain comprises, has, or consists of, 25 nucleotides (e.g., 25 consecutive nucleotides) having complementarity with the target domain, e.g., the targeting domain is 25 nucleotides in length.

In an embodiment, the targeting domain comprises, has, or consists of, 26 nucleotides (e.g., 26 consecutive nucleotides) having complementarity with the target domain, e.g., the targeting domain is 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGAGCUAGAAAUAGCAAGUUAAAAUAAGG CUAGUCCGUUAUCAACUUGAAAAAGUGGCACCGAGUCGGUGCUUUUUU (SEQ ID NO: 45). In an embodiment, the unimolecular, or chimeric, gRNA molecule is a S. pyogenes gRNA molecule.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGUACUCUGGAAACAGAAUCUACUAAAAC AAGGCAAAAUGCCGUGUUUAUCUCGUCAACUUGUUGGCGAGAUUUUUU (SEQ ID NO: 40). In an embodiment, the unimolecular, or chimeric, gRNA molecule is a S. aureus gRNA molecule.

The sequences and structures of exemplary chimeric gRNAs are also shown in FIGS. 1H-1I.

Exemplary Modular gRNAs

In an embodiment, a modular gRNA comprises:
a first strand comprising, preferably from 5′ to 3′;
   a targeting domain, e.g., comprising 15, 16, 17, 18, 19, 20, 21, 22, 23, 24, 25, or 26 nucleotides;
   a first complementarity domain; and
   a second strand, comprising, preferably from 5′ to 3′:
   optionally a 5′ extension domain;
   a second complementarity domain;
   a proximal domain; and
   a tail domain,
wherein:

(a) the proximal and tail domain, when taken together, comprise

at least 15, 18, 20, 25, 30, 31, 35, 40, 45, 49, 50, or 53 nucleotides;

(b) there are at least 15, 18, 20, 25, 30, 31, 35, 40, 45, 49, 50, or 53 nucleotides 3′ to the last nucleotide of the second complementarity domain; or

(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein.

In an embodiment, the proximal and tail domain, when taken together, comprise at least 15, 18, 20, 25, 30, 31, 35, 40, 45, 49, 50, or 53 nucleotides.

In an embodiment, there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length.

In an embodiment, the targeting domain comprises, has, or consists of, 17 nucleotides (e.g., 17 consecutive nucleotides) having complementarity with the target domain, e.g., the targeting domain is 17 nucleotides in length.

In an embodiment, the targeting domain comprises, has, or consists of, 18 nucleotides (e.g., 18 consecutive nucleotides) having complementarity with the target domain, e.g., the targeting domain is 18 nucleotides in length.

In an embodiment, the targeting domain comprises, has, or consists of, 19 nucleotides (e.g., 19 consecutive nucleotides) having complementarity with the target domain, e.g., the targeting domain is 19 nucleotides in length.

In an embodiment, the targeting domain comprises, has, or consists of, 20 nucleotides (e.g., 20 consecutive nucleotides) having complementarity with the target domain, e.g., the targeting domain is 20 nucleotides in length.

In an embodiment, the targeting domain comprises, has, or consists of, 21 nucleotides (e.g., 21 consecutive nucleotides) having complementarity with the target domain, e.g., the targeting domain is 21 nucleotides in length.

In an embodiment, the targeting domain comprises, has, or consists of, 22 nucleotides (e.g., 22 consecutive nucleotides) having complementarity with the target domain, e.g., the targeting domain is 22 nucleotides in length.

In an embodiment, the targeting domain comprises, has, or consists of, 23 nucleotides (e.g., 23 consecutive nucleotides) having complementarity with the target domain, e.g., the targeting domain is 23 nucleotides in length.

In an embodiment, the targeting domain comprises, has, or consists of, 24 nucleotides (e.g., 24 consecutive nucleotides) having complementarity with the target domain, e.g., the targeting domain is 24 nucleotides in length.

In an embodiment, the targeting domain comprises, has, or consists of, 25 nucleotides (e.g., 25 consecutive nucleotides) having complementarity with the target domain, e.g., the targeting domain is 25 nucleotides in length.

In an embodiment, the targeting domain comprises, has, or consists of, 26 nucleotides (e.g., 26 consecutive nucleotides) having complementarity with the target domain, e.g., the targeting domain is 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

II. Methods for Designing gRNAs

Methods for designing gRNAs are described herein, including methods for selecting, designing and validating target domains. Exemplary targeting domains are also provided herein. Targeting Domains discussed herein can be incorporated into the gRNAs described herein.

Methods for selection and validation of target sequences as well as off-target analyses are described, e.g., in Mali et al., 2013 SCIENCE 339(6121): 823-826; Hsu et al. NAT BIOTECHNOL, 31(9): 827-32; Fu et al., 2014 NAT BIOTECHNOL, doi: 10.1038/nbt.2808. PubMed PMID: 24463574; Heigwer et al., 2014 NAT METHODS 11(2):122-3. doi: 10.1038/nmeth.2812. PubMed PMID: 24481216; Bae et al., 2014 BIOINFORMATICS PubMed PMID: 24463181; Xiao A et al., 2014 BIOINFORMATICS PubMed PMID: 24389662.

For example, a software tool can be used to optimize the choice of gRNA within a user's target sequence, e.g., to minimize total off-target activity across the genome. Off target activity may be other than cleavage. For each possible gRNA choice using S. pyogenes Cas9, the tool can identify all off-target sequences (preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate gRNA molecules can be evaluated by art-known methods or as described in Section IV herein.

Guide RNAs (gRNAs) for use with S. pyogenes, S. aureus and N. meningitidis Cas9s were identified using a DNA sequence searching algorithm. Guide RNA design was carried out using a custom guide RNA design software based on the public tool cas-offinder (reference: Cas-OFFinder: a fast and versatile algorithm that searches for potential off-target sites of Cas9 RNA-guided endonucleases, Bioinformatics. 2014 Feb. 17. Bae S, Park J, Kim J S. PMID:24463181). Said custom guide RNA design software scores guides after calculating their genomewide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, gRNAs were ranked into tiers based on their distance to the target site, their orthogonality or presence of a 5′ G (based on identification of close matches in the human genome containing a relevant PAM, e.g., in the case of S. pyogenes, a NGG PAM, in the case of S. aureus, NNGRR (e.g, a NNGRRT or NNGRRV) PAM, and in the case of N. meningitides, a NNNNGATT or NNNNGCTT PAM. Orthogonality refers to the number of sequences in the human genome that contain a minimum number of mismatches to the target sequence. A “high level of orthogonality” or “good orthogonality” may, for example, refer to 20-mer gRNAs that have no identical sequences in the human genome besides the intended target, nor any sequences that contain one or two mismatches in the target sequence. Targeting domains with good orthogonality are selected to minimize off-target DNA cleavage.

As an example, for S. pyogenes and N. meningitides targets, 17-mer, or 20-mer gRNAs were designed. As another example, for S. aureus targets, 18-mer, 19-mer, 20-mer, 21-mer, 22-mer, 23-mer and 24-mer gRNAs were designed. Targeting domains, disclosed herein, may comprise the 17-mer described in Tables 1A-1F, 2A-2C, 3A-3E, 4A-4C, 5A-5C, 6A-6E or 7A-7C, e.g., the targeting domains of 18 or more nucleotides may comprise the 17-mer gRNAs described in Tables 1A-1F, 2A-2C, 3A-3E, 4A-4C, 5A-5C, 6A-6E or 7A-7C. Targeting domains, disclosed herein, may comprises the 18-mer described in Tables 1A-1F, 2A-2C, 3A-3E, 4A-4C, 5A-5C, 6A-6E or 7A-7C, e.g., the targeting domains of 19 or more nucleotides may comprise the 18-mer gRNAs described in Tables 1A-1F, 2A-2C, 3A-3E, 4A-4C, 5A-5C, 6A-6E or 7A-7C. Targeting domains, disclosed herein, may comprises the 19-mer described in Tables 1A-1F, 2A-2C, 3A-3E, 4A-4C, 5A-5C, 6A-6E or 7A-7C, e.g., the targeting domains of 20 or more nucleotides may comprise the 19-mer gRNAs described in Tables 1A-1F, 2A-2C, 3A-3E, 4A-4C, 5A-5C, 6A-6E or 7A-7C. Targeting domains, disclosed herein, may comprises the 20-mer gRNAs described in Tables 1A-1F, 2A-2C, 3A-3E, 4A-4C, 5A-5C, 6A-6E or 7A-7C e.g., the targeting domains of 21 or more nucleotides may comprise the 20-mer gRNAs described in Tables 1A-1F, 2A-2C, 3A-3E, 4A-4C, 5A-5C, 6A-6E or 7A-7C. Targeting domains, disclosed herein, may comprises the 21-mer described in Tables 1A-1F, 2A-2C, 3A-3E, 4A-4C, 5A-5C, 6A-6E or 7A-7C e.g., the targeting domains of 22 or more nucleotides may comprise the 21-mer gRNAs described in Tables 1A-1F, 2A-2C, 3A-3E, 4A-4C, 5A-5C, 6A-6E or 7A-7C. Targeting domains, disclosed herein, may comprises the 22-mer described in Tables 1A-1F, 2A-2C, 3A-3E, 4A-4C, 5A-5C, 6A-6E or 7A-7C, e.g., the targeting domains of 23 or more nucleotides may comprise the 22-mer gRNAs described in Tables 1A-1F, 2A-2C, 3A-3E, 4A-4C, 5A-5C, 6A-6E or 7A-7C. Targeting domains, disclosed herein, may comprises the 23-mer described in Tables 1A-1F, 2A-2C, 3A-3E, 4A-4C, 5A-5C, 6A-6E or 7A-7C e.g., the targeting domains of 24 or more nucleotides may comprise the 23-mer gRNAs described in Tables 1A-1F, 2A-2C, 3A-3E, 4A-4C, 5A-5C, 6A-6E or 7A-7C. Targeting domains, disclosed herein, may comprises the 24-mer described in Tables 1A-1F, 2A-2C, 3A-3E, 4A-4C, 5A-5C, 6A-6E or 7A-7C, e.g., the targeting domains of 25 or more nucleotides may comprise the 24-mer gRNAs described in Tables 1A-1F, 2A-2C, 3A-3E, 4A-4C, 5A-5C, 6A-6E or 7A-7C. gRNAs were identified for both single-gRNA nuclease cleavage and for a dual-gRNA paired “nickase” strategy. Criteria for selecting gRNAs and the determination for which gRNAs can be used for which strategy is based on several considerations:

gRNA pairs should be oriented on the DNA such that PAMs are facing out and cutting with the D10A Cas9 nickase will result in 5′ overhangs.

An assumption that cleaving with dual nickase pairs will result in deletion of the entire intervening sequence at a reasonable frequency. However, it will also often result in indel mutations at the site of only one of the gRNAs. Candidate pair members can be tested for how efficiently they remove the entire sequence versus just causing indel mutations at the site of one gRNA.

The Targeting Domains discussed herein can be incorporated into the gRNAs described herein.

Strategies to Identify gRNAs for S. pyogenes, S. Aureus, and N. meningitides to Knock Out the CCR5 Gene

As an example, two strategies were utilized to identify gRNAs for use with S. pyogenes, S. aureus and N. meningitidis Cas9 enzymes.

In one strategy, gRNAs were designed for use with S. pyogenes Cas9 enzymes (Tables 1A-1D). While it can be desirable to have gRNAs start with a 5′ G, this requirement was relaxed for some gRNAs in tier 1 in order to identify guides in the correct orientation, within a reasonable distance to the mutation and with a high level of orthogonality. In order to find a pair for the dual-nickase strategy it was necessary to either extend the distance from the mutation or remove the requirement for the 5′G. For selection of tier 2 gRNAs, the distance restriction was relaxed in some cases such that a longer sequence was scanned, but the 5′G was required for all gRNAs. Whether or not the distance requirement was relaxed depended on how many sites were found within the original search window. Tier 3 uses the same distance restriction as tier 2, but removes the requirement for a 5′G. Note that tiers are non-inclusive (each gRNA is listed only once). Tier 4 gRNAs were selected based on location in coding sequence of gene.

As discussed above, gRNAs were identified for single-gRNA nuclease cleavage as well as for a dual-gRNA paired “nickase” strategy, as indicated.

gRNAs for use with the Neisseria meningitidis and Staphylococcus aureus Cas9s were identified manually by scanning genomic DNA sequence for the presence of PAM sequences. These gRNAs were not separated into tiers, but are provided in single lists for each species (Table 1E for S. aureus and Table 1F for N. meningitides).

As discussed above, gRNAs were identified for single-gRNA nuclease cleavage as well as for a dual-gRNA paired “nickase” strategy, as indicated.

In another strategy, gRNAs were designed for use with S. pyogenes, S. aureus and N. meningitidis Cas9 enzymes. The gRNAs were identified and ranked into 3 tiers for S. pyogenes (Tables 2A-2C). The targeting domain to be used with S. pyogenes Cas9 enzymes for tier 1 gRNA molecules were selected based on (1) distance to a target site (e.g., start codon), e.g., within 500 bp (e.g., downstream) of the target site (e.g., start codon) and (2) a high level of orthogonality. The targeting domain to be used with S. pyogenes Cas9 enzymes for tier 2 gRNA molecules were selected based on (1) distance to the target site (e.g., start codon), e.g., within 500 bp (e.g., downstream) of the target site (e.g., start codon). The targeting domain to be used with S. pyogenes Cas9 enzymes for tier 3 gRNA molecules were selected based on distance to the target site (e.g., start codon), e.g., within reminder of the coding sequence, e.g., downstream of the first 500 bp of coding sequence (e.g., anywhere from +500 (relative to the start codon) to the stop codon). The gRNAs were identified and ranked into 5 tiers for S. aureus, when the relevant PAM was NNGRRT or NNGRRV (Tables 3A-3E). The targeting domain to be used with S. aureus Cas9 enzymes for tier 1 gRNA molecules were selected based on (1) distance to the target site (e.g., start codon), e.g., within 500 bp (e.g., downstream) of the target site (e.g., start codon), (2) a high level of orthogonality, and (3) PAM is NNGRRT. The targeting domain to be used with S. aureus Cas9 enzymes for tier 2 gRNA molecules were selected based on (1) distance to the target site (e.g., start codon), e.g., within 500 bp (e.g., downstream) of the target site (e.g., start codon), and (2) PAM is NNGRRT. The targeting domain to be used with S. aureus Cas9 enzymes for tier 3 gRNA molecules were selected based on (1) distance to a the target site (e.g., start codon), e.g., within 500 bp (e.g., downstream) of the target site (e.g., start codon), and (2) PAM is NNGRRV. The targeting domain to be used with S. aureus Cas9 enzymes for tier 4 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon), and (2) PAM is NNGRRT. The targeting domain to be used with S. aureus Cas9 enzymes for tier 5 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon), and (2) PAM is NNGRRV. The gRNAs were identified and ranked into 3 tiers for N. meningitidis (Tables 4A-4C). The targeting domain to be used with N. meningitidis Cas9 enzymes for tier 1 gRNA molecules were selected based on (1) distance to the target site, e.g., within 500 bp (e.g., downstream) of the target site (e.g., start codon) and (2) a high level of orthogonality. The targeting domain to be used with N. meningitidis Cas9 enzymes for tier 2 gRNA molecules were selected based on (1) distance to the target site (e.g., start codon), e.g., within 500 bp (e.g., downstream) of the target site (e.g., start codon). The targeting domain to be used with N. meningitidis Cas9 enzymes for tier 3 gRNA molecules were selected based on distance to the target site (e.g., start codon), e.g., within reminder of the coding sequence, e.g., downstream of the first 500 bp of coding sequence (e.g., anywhere from +500 (relative to the start codon) to the stop codon). Note that tiers are non-inclusive (each gRNA is listed only once for the strategy). In certain instances, no gRNA was identified based on the criteria of the particular tier.

In an embodiment, when a single gRNA molecule is used to target a Cas9 nickase to create a single strand break in close proximity to the CCR5 target position, e.g., the gRNA is used to target either upstream of (e.g., within 500 bp, e.g., within 200 bp upstream of the CCR5 target position), or downstream of (e.g., within 500 bp, e.g., within 200 bp downstream of the CCR5 target position) in the CCR5 gene.

In an embodiment, when a single gRNA molecule is used to target a Cas9 nuclease to create a double strand break to in close proximity to the CCR5 target position, e.g., the gRNA is used to target either upstream of (e.g., within 500 bp, e.g., within 200 bp upstream of the CCR5 target position), or downstream of (e.g., within 500 bp, e.g., within 200 bp downstream of the CCR5 target position) in the CCR5 gene.

In an embodiment, dual targeting is used to create two double strand breaks to in close proximity to the mutation, e.g., the gRNA is used to target either upstream of (e.g., within 500 bp, e.g., within 200 bp upstream of the CCR5 target position), or downstream of (e.g., within 500 bp, e.g., within 200 bp downstream of the CCR5 target position) in the CCR5 gene. In an embodiment, the first and second gRNAs are used to target two Cas9 nucleases to flank, e.g., the first of gRNA is used to target upstream of (e.g., within 500 bp, e.g., within 200 bp upstream of the CCR5 target position), and the second gRNA is used to target downstream of (e.g., within 500 bp, e.g., within 200 bp downstream of the CCR5 target position) in the CCR5 gene.

In an embodiment, dual targeting is used to create a double strand break and a pair of single strand breaks to delete a genomic sequence including the CCR5 target position. In an embodiment, the first, second and third gRNAs are used to target one Cas9 nuclease and two Cas9 nickases to flank, e.g., the first gRNA that will be used with the Cas9 nuclease is used to target upstream of (e.g., within 500 bp, e.g., within 200 bp upstream of the CCR5 target position) or downstream of (e.g., within 500 bp, e.g., within 200 bp downstream of the CCR5 target position), and the second and third gRNAs that will be used with the Cas9 nickase pair are used to target the opposite side of the mutation (e.g., within 200 bp upstream or downstream of the CCR5 target position) in the CCR5 gene.

In an embodiment, when four gRNAs (e.g., two pairs) are used to target four Cas9 nickases to create four single strand breaks to delete genomic sequence including the mutation, the first pair and second pair of gRNAs are used to target four Cas9 nickases to flank, e.g., the first pair of gRNAs are used to target upstream of (e.g., within 500 bp, e.g., within 200 bp upstream of the CCR5 target position), and the second pair of gRNAs are used to target downstream of (e.g., within 500 bp, e.g., within 200 bp downstream of the CCR5 target position) in the CCR5 gene.

Strategies to Identify gRNAs for S. pyogenes, S. Aureus, and N. meningitides to Knock Down the CCR5 Gene

In yet another strategy, gRNAs were designed for use with S. pyogenes, S. aureus and N. meningitidis Cas9 enzymes. The gRNAs were identified and ranked into 3 tiers for S. pyogenes (Tables 5A-5C). The targeting domain to be used with S. pyogenes Cas9 enzymes for tier 1 gRNA molecules were selected based on (1) distance to a target site (e.g., the transcription start site), e.g., within 500 bp (e.g., upstream or downstream) of the target site (e.g., the transcription start site) and (2) a high level of orthogonality. The targeting domain to be used with S. pyogenes Cas9 enzymes for tier 2 gRNA molecules were selected based on (1) distance to the target site (e.g., the transcription start site), e.g., within 500 bp (e.g., upstream or downstream) of the target site (e.g., the transcription start site). The targeting domain to be used with S. pyogenes Cas9 enzymes for tier 3 gRNA molecules were selected based on distance to the target site (e.g., the transcription start site), e.g., within the additional 500 bp upstream and downstream of the transcription start site (i.e., extending to 1 kb upstream and downstream of the transcription start site. The gRNAs were identified and ranked into 5 tiers for S. aureus, when the relevant PAM was NNGRRT or NNGRRV (Tables 6A-6E). The targeting domain to be used with S. aureus Cas9 enzymes for tier 1 gRNA molecules were selected based on (1) distance to the target site (e.g., the transcription start site), e.g., within 500 bp (e.g., upstream or downstream) of the target site (e.g., the transcription start site), (2) a high level of orthogonality, and (3) PAM is NNGRRT. The targeting domain to be used with S. aureus Cas9 enzymes for tier 2 gRNA molecules were selected based on (1) distance to the target site (e.g., the transcription start site), e.g., within 500 bp (e.g., upstream or downstream) of the target site (e.g., the transcription start site), and (2) PAM is NNGRRT. The targeting domain to be used with S. aureus Cas9 enzymes for tier 3 gRNA molecules were selected based on (1) distance to a target site (e.g., the transcription start site), e.g., within 500 bp (e.g., upstream or downstream) of the target site (e.g., the transcription start site), and (2) PAM is NNGRRV. The targeting domain to be used with S. aureus Cas9 enzymes for tier 4 gRNA molecules were selected based on (1) distance to the target site (e.g., the transcription start site), e.g., within the additional 500 bp upstream and downstream of the transcription start site (i.e., extending to 1 kb upstream and downstream of the transcription start site, and (2) PAM is NNGRRT. The targeting domain to be used with S. aureus Cas9 enzymes for tier 5 gRNA molecules were selected based on (1) distance to the target site (e.g., the transcription start site), e.g., within the additional 500 bp upstream and downstream of the transcription start site (i.e., extending to 1 kb upstream and downstream of the transcription start site, and (2) PAM is NNGRRV. The gRNAs were identified and ranked into 3 tiers for N. meningitidis (Tables 7A-7C). The targeting domain to be used with N. meningitidis Cas9 enzymes for tier 1 gRNA molecules were selected based on (1) distance to a target site (e.g., the transcription start site), e.g., within 500 bp (e.g., upstream or downstream) of the target site (e.g., the transcription start site) and (2) a high level of orthogonality. The targeting domain to be used with N. meningitidis Cas9 enzymes for tier 2 gRNA molecules were selected based on (1) distance to the target site (e.g., the transcription start site), e.g., within 500 bp (e.g., upstream or downstream) of the target site (e.g., the transcription start site). The targeting domain to be used with N. meningitidis Cas9 enzymes for tier 3 gRNA molecules were selected based on distance to the target site (e.g., the transcription start site), e.g., within the additional 500 bp upstream and downstream of the transcription start site (i.e., extending to 1 kb upstream and downstream of the transcription start site. Note that tiers are non-inclusive (each gRNA is listed only once for the strategy). In certain instances, no gRNA was identified based on the criteria of the particular tier.

Any of the targeting domains in the tables described herein can be used with a Cas9 nickase molecule to generate a single strand break.

Any of the targeting domains in the tables described herein can be used with a Cas9 nuclease molecule to generate a double strand break.

In an embodiment, dual targeting (e.g., dual nicking) is used to create two nicks on opposite DNA strands by using S. pyogenes, S. aureus and N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

When two gRNAs designed for use to target two Cas9 molecules, one Cas9 can be one species, the second Cas9 can be from a different species. Both Cas9 species are used to generate a single or double-strand break, as desired.

### Exemplary Targeting Domains

Table 1A provides exemplary targeting domains for knocking out the CCR5 gene selected according to first tier parameters, and are selected based on the presence of a 5′ G (except for CCR5-51, -52, -60, -63, -64 and -66), close proximity to the start codon and orthogonality in the human genome. In an embodiment, the targeting domain is the exact complement of the target domain. Any of the targeting domains in the table can be used with a Cas9 molecule (e.g., a S. pyogenes Cas9 molecule) that gives double stranded cleavage. Any of the targeting domains in the table can be used with Cas9 single-stranded break nucleases (nickases) (e.g., S. pyogenes Cas9 single-stranded break nucleases). In an embodiment, dual targeting is used to create two nicks. When selecting gRNAs for use in a nickase pair, one gRNA targets a domain in the complementary strand and the second gRNA targets a domain in the non-complementary strand. In an embodiment, two 20-mer guide RNAs are used to target two S. pyogenes Cas9 nucleases or two S. pyogenes Cas9 nickases, e.g., CCR5-63 and CCR5-49, or CCR5-63 and CCR5-41 are used. In an embodiment, two 17-mer guide RNAs are used to target two Cas9 nucleases or two Cas9 nickases, e.g., CCR5-4 and CCR5-3 are used.

TABLE 1A
1st Tier

| | | | | SEQ |
|---|---|---|---|---|
| gRNA | DNA | | Target Site | ID |
| Name | Strand | Targeting Domain | Length | NO |
| CCR5-66 | − | CCUGCCUCCGCUCUACUCAC | 20 | 387 |
| CCR5-43 | − | GCUGCCGCCCAGUGGGACUU | 20 | 388 |
| CCR5-51 | − | ACAAUGUGUCAACUCUUGAC | 20 | 389 |
| CCR5-58 | − | GGUGACAAGUGUGAUCACUU | 20 | 390 |
| CCR5-60 | + | CCAGGUACCUAUCGAUUGUC | 20 | 391 |
| CCR5-63 | + | CUUCACAUUGAUUUUUUGGC | 20 | 392 |
| CCR5-47 | + | GCAGCAUAGUGAGCCCAGAA | 20 | 393 |
| CCR5-45 | + | GGUACCUAUCGAUUGUCAGG | 20 | 394 |
| CCR5-49 | + | GUGAGUAGAGCGGAGGCAGG | 20 | 395 |
| CCR5-1 | − | GCCUCCGCUCUACUCAC | 17 | 396 |
| CCR5-3 | − | GCCGCCCAGUGGGACUU | 17 | 397 |
| CCR5-52 | − | AUGUGUCAACUCUUGAC | 17 | 398 |
| CCR5-10 | − | GACAAUCGAUAGGUACC | 17 | 399 |
| CCR5-64 | + | CACAUUGAUUUUUUGGC | 17 | 400 |
| CCR5-4 | + | GCAUAGUGAGCCCAGAA | 17 | 401 |
| CCR5-14 | + | GGUACCUAUCGAUUGUC | 17 | 402 |

Table 1B provides exemplary targeting domains for knocking out the CCR5 gene selected according to the second tier parameters and are selected based on the presence of a 5′ G and close proximity to the start codon. In an embodiment, the targeting domain is the exact complement of the target domain. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks.

TABLE 1B
2nd Tier

| | | | Target | |
|---|---|---|---|---|
| gRNA | DNA | | Site | SEQ |
| Name | Strand | Targeting Domain | Length | ID NO |
| CCR5-5 | + | GAAAAACAGGUCAGAGA | 17 | 403 |
| CCR5-13 | − | GACAAGUGUGAUCACUU | 17 | 404 |
| CCR5-85 | − | GACAAGUGUGAUCACUUGGG | 20 | 405 |
| CCR5-12 | − | GACGGUCACCUUUGGGG | 17 | 406 |
| CCR5-8 | + | GAGCGGAGGCAGGAGGC | 17 | 407 |
| CCR5-11 | − | GCCAGGACGGUCACCUU | 17 | 408 |
| CCR5-6 | + | GCCUUUUGCAGUUUAUC | 17 | 409 |
| CCR5-59 | − | GCUGUGUUUGCGUCUCUCCC | 20 | 410 |
| CCR5-9 | + | GCUUCACAUUGAUUUUU | 17 | 411 |
| CCR5-48 | + | GGACAGUAAGAAGGAAAAAC | 20 | 412 |
| CCR5-46 | + | GGCAGCAUAGUGAGCCCAGA | 20 | 413 |
| CCR5-41 | − | GGUGUUCAUCUUUGGUUUUG | 20 | 414 |
| CCR5-50 | + | GUAGAGCGGAGGCAGGAGGC | 20 | 415 |
| CCR5-7 | + | GUGAGUAGAGCGGAGGC | 17 | 416 |
| CCR5-42 | − | GUGUUCAUCUUUGGUUUUGU | 20 | 417 |
| CCR5-129 | − | GUGUUUGCGUCUCUCCC | 17 | 418 |
| CCR5-2 | − | GUUCAUCUUUGGUUUUG | 17 | 419 |
| CCR5-79 | − | GUUUGCUUUAAAAGCCAGGA | 20 | 420 |

Table 1C provides exemplary targeting domains for knocking out the CCR5 gene selected according to the third tier parameters and are selected based on close proximity to the start codon. In an embodiment, the targeting domain is the exact complement of the target domain. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks.

TABLE 1C
3rd Tier

| | | | Target | |
|---|---|---|---|---|
| gRNA | DNA | | Site | SEQ |
| Name | Strand | Targeting Domain | Length | ID NO |
| CCR5-87 | + | AAAACAGGUCAGAGAUGGCC | 20 | 421 |
| CCR5-80 | − | AAAGCCAGGACGGUCACCUU | 20 | 422 |
| CCR5-130 | + | AACACCAGUGAGUAGAG | 17 | 423 |
| CCR5-88 | + | AACACCAGUGAGUAGAGCGG | 20 | 424 |
| CCR5-81 | − | AAGCCAGGACGGUCACCUUU | 20 | 425 |
| CCR5-89 | + | AAGGAAAAACAGGUCAGAGA | 20 | 426 |
| CCR5-127 | − | AAGUGUGAUCACUUGGG | 17 | 427 |
| CCR5-86 | − | AAGUGUGAUCACUUGGGUGG | 20 | 428 |
| CCR5-90 | + | ACACAGCAUGGACGACAGCC | 20 | 429 |
| CCR5-119 | − | ACAGGGCUCUAUUUUAU | 17 | 430 |
| CCR5-131 | + | ACAGGUCAGAGAUGGCC | 17 | 431 |
| CCR5-132 | + | ACAUUGAUUUUUUGGCA | 17 | 432 |
| CCR5-133 | + | ACCAGUGAGUAGAGCGG | 17 | 433 |
| CCR5-134 | + | ACCUAUCGAUUGUCAGG | 17 | 434 |
| CCR5-115 | − | ACUAUGCUGCCGCCCAG | 17 | 435 |
| CCR5-135 | + | ACUUGUCACCACCCCAA | 17 | 436 |
| CCR5-136 | + | AGAAGGGGACAGUAAGA | 17 | 437 |
| CCR5-137 | + | AGAGCGGAGGCAGGAGG | 17 | 438 |
| CCR5-138 | + | AGAUGGCCAGGUUGAGC | 17 | 439 |
| CCR5-139 | + | AGCAUAGUGAGCCCAGA | 17 | 440 |
| CCR5-82 | − | AGCCAGGACGGUCACCUUUG | 20 | 441 |
| CCR5-65 | + | AGUAGAGCGGAGGCAGG | 17 | 442 |
| CCR5-91 | + | AGUAGAGCGGAGGCAGGAGG | 20 | 443 |
| CCR5-92 | + | AUGAACACCAGUGAGUAGAG | 20 | 444 |
| CCR5-141 | + | AUUUCCAAAGUCCCACU | 17 | 445 |
| CCR5-93 | + | AUUUCCAAAGUCCCACUGGG | 20 | 446 |
| CCR5-76 | − | CAAUGUGUCAACUCUUGACA | 20 | 447 |
| CCR5-94 | + | CACACUUGUCACCACCCCAA | 20 | 448 |
| CCR5-95 | + | CACCCCAAAGGUGACCGUCC | 20 | 449 |
| CCR5-96 | + | CAGAGAUGGCCAGGUUGAGC | 20 | 450 |
| CCR5-97 | + | CAGCAUAGUGAGCCCAGAAG | 20 | 451 |
| CCR5-143 | + | CAGCAUGGACGACAGCC | 17 | 452 |
| CCR5-125 | − | CAGGACGGUCACCUUUG | 17 | 453 |
| CCR5-83 | − | CAGGACGGUCACCUUUGGGG | 20 | 454 |
| CCR5-144 | + | CAGUAAGAAGGAAAAAC | 17 | 455 |
| CCR5-145 | + | CAUAGUGAGCCCAGAAG | 17 | 456 |
| CCR5-107 | − | CAUCAAUUAUUAUACAU | 17 | 457 |
| CCR5-112 | − | CAUCUACCUGCUCAACC | 17 | 458 |
| CCR5-124 | − | CCAGGACGGUCACCUUU | 17 | 459 |
| CCR5-98 | + | CCAGUGAGUAGAGCGGAGGC | 20 | 460 |
| CCR5-146 | + | CCCAAAGGUGACCGUCC | 17 | 461 |
| CCR5-99 | + | CCCAGAAGGGGACAGUAAGA | 20 | 462 |
| CCR5-57 | − | CCUGACAAUCGAUAGGUACC | 20 | 463 |
| CCR5-73 | − | CCUUCUUACUGUCCCCUUCU | 20 | 464 |
| CCR5-116 | − | CUAUGCUGCCGCCCAGU | 17 | 465 |
| CCR5-74 | − | CUCACUAUGCUGCCGCCCAG | 20 | 466 |
| CCR5-78 | − | CUGUGUUUGCUUUAAAAGCC | 20 | 467 |
| CCR5-100 | + | CUUUUAAAGCAAACACAGCA | 20 | 468 |
| CCR5-101 | + | UAAUAAUUGAUGUCAUAGAU | 20 | 469 |
| CCR5-147 | + | UAAUUGAUGUCAUAGAU | 17 | 470 |
| CCR5-68 | − | UACUCACUGGUGUUCAUCUU | 20 | 471 |
| CCR5-148 | + | UAUUUCCAAAGUCCCAC | 17 | 472 |
| CCR5-77 | − | UAUUUUAUAGGCUUCUUCUC | 20 | 473 |
| CCR5-75 | − | UCACUAUGCUGCCGCCCAGU | 20 | 474 |
| CCR5-108 | − | UCACUGGUGUUCAUCUU | 17 | 475 |
| CCR5-62 | + | UCAGCCUUUUGCAGUUUAUC | 20 | 476 |
| CCR5-55 | − | UCAUCCUCCUGACAAUCGAU | 20 | 477 |
| CCR5-70 | − | UCAUCCUGAUAAACUGCAAA | 20 | 478 |
| CCR5-149 | + | UCCAAAGUCCCACUGGG | 17 | 479 |
| CCR5-121 | − | UCCUCCUGACAAUCGAU | 17 | 480 |
| CCR5-111 | − | UCCUGAUAAACUGCAAA | 17 | 481 |
| CCR5-72 | − | UCCUUCUUACUGUCCCCUUC | 20 | 482 |
| CCR5-114 | − | UCUUACUGUCCCCUUCU | 17 | 483 |
| CCR5-126 | − | UGACAAGUGUGAUCACU | 17 | 484 |
| CCR5-67 | − | UGACAUCAAUUAUUAUACAU | 20 | 485 |
| CCR5-71 | − | UGACAUCUACCUGCUCAACC | 20 | 486 |
| CCR5-150 | + | UGCAGUUUAUCAGGAUG | 17 | 487 |
| CCR5-123 | − | UGCUUUAAAAGCCAGGA | 17 | 488 |
| CCR5-84 | − | UGGUGACAAGUGUGAUCACU | 20 | 489 |
| CCR5-69 | − | UGGUUUUGUGGGCAACAUGC | 20 | 490 |
| CCR5-102 | + | UGUAUUUCCAAAGUCCCACU | 20 | 491 |
| CCR5-128 | − | UGUGAUCACUUGGGUGG | 17 | 492 |
| CCR5-118 | − | UGUGUCAACUCUUGACA | 17 | 493 |
| CCR5-122 | − | UGUUUGCUUUAAAAGCC | 17 | 494 |
| CCR5-151 | + | UUAAAGCAAACACAGCA | 17 | 495 |
| CCR5-103 | + | UUCACAUUGAUUUUUUGGCA | 20 | 496 |
| CCR5-109 | − | UUCAUCUUUGGUUUUGU | 17 | 497 |
| CCR5-113 | − | UUCUUACUGUCCCCUUC | 17 | 498 |
| CCR5-53 | − | UUGACAGGGCUCUAUUUUAU | 20 | 499 |
| CCR5-104 | + | UUGUAUUUCCAAAGUCCCAC | 20 | 500 |
| CCR5-120 | − | UUUAUAGGCUUCUUCUC | 17 | 501 |
| CCR5-105 | + | UUUGCUUCACAUUGAUUUUU | 20 | 502 |
| CCR5-106 | + | UUUUGCAGUUUAUCAGGAUG | 20 | 503 |
| CCR5-110 | − | UUUUGUGGGCAACAUGC | 17 | 504 |

Table 1D provides exemplary targeting domains for knocking out the CCR5 gene selected according to the fourth tier parameters and are selected on location in coding sequence of gene. In an embodiment, the targeting domain is the exact complement of the target domain. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks.

TABLE 1D
4th Tier

| | | | Target | |
|---|---|---|---|---|
| gRNA | DNA | | Site | SEQ |
| Name | Strand | Targeting Domain | Length | ID NO |
| CCR5-152 | − | CAUACAGUCAGUAUCAAUUC | 20 | 505 |
| CCR5-153 | − | GACAUUAAAGAUAGUCAUCU | 20 | 506 |
| CCR5-154 | − | ACAUUAAAGAUAGUCAUCUU | 20 | 507 |
| CCR5-155 | − | CAUUAAAGAUAGUCAUCUUG | 20 | 508 |
| CCR5-156 | − | AAAGAUAGUCAUCUUGGGGC | 20 | 509 |
| CCR5-157 | − | GGUCCUGCCGCUGCUUGUCA | 20 | 510 |
| CCR5-158 | − | UGUCAUGGUCAUCUGCUACU | 20 | 511 |
| CCR5-159 | − | GUCAUGGUCAUCUGCUACUC | 20 | 512 |
| CCR5-160 | − | GAAUCCUAAAAACUCUGCUU | 20 | 513 |
| CCR5-161 | − | GGUGUCGAAAUGAGAAGAAG | 20 | 514 |
| CCR5-162 | − | GAAAUGAGAAGAAGAGGCAC | 20 | 515 |
| CCR5-163 | − | AAAUGAGAAGAAGAGGCACA | 20 | 516 |
| CCR5-164 | − | AGAAGAGGCACAGGGCUGUG | 20 | 517 |
| CCR5-165 | − | UGAUUGUUUAUUUUCUCUUC | 20 | 518 |
| CCR5-166 | − | GAUUGUUUAUUUUCUCUUCU | 20 | 519 |
| CCR5-167 | − | CCUUCUCCUGAACACCUUCC | 20 | 520 |
| CCR5-168 | − | AACACCUUCCAGGAAUUCUU | 20 | 521 |
| CCR5-169 | − | AUAAUUGCAGUAGCUCUAAC | 20 | 522 |
| CCR5-170 | − | UUGCAGUAGCUCUAACAGGU | 20 | 523 |
| CCR5-171 | − | CAGGUUGGACCAAGCUAUGC | 20 | 524 |
| CCR5-172 | − | AUGCAGGUGACAGAGACUCU | 20 | 525 |
| CCR5-173 | − | UGCAGGUGACAGAGACUCUU | 20 | 526 |
| CCR5-174 | − | CCCAUCAUCUAUGCCUUUGU | 20 | 527 |
| CCR5-175 | − | CCAUCAUCUAUGCCUUUGUC | 20 | 528 |
| CCR5-176 | − | CAUCAUCUAUGCCUUUGUCG | 20 | 529 |
| CCR5-177 | − | CUGUUCUAUUUUCCAGCAAG | 20 | 530 |
| CCR5-178 | − | UCAGUUUACACCCGAUCCAC | 20 | 531 |
| CCR5-179 | − | CAGUUUACACCCGAUCCACU | 20 | 532 |
| CCR5-180 | − | AGUUUACACCCGAUCCACUG | 20 | 533 |
| CCR5-181 | − | CACCCGAUCCACUGGGGAGC | 20 | 534 |
| CCR5-182 | − | UGGGGAGCAGGAAAUAUCUG | 20 | 535 |
| CCR5-183 | − | GGGGAGCAGGAAAUAUCUGU | 20 | 536 |
| CCR5-184 | − | AUAUCUGUGGGCUUGUGACA | 20 | 537 |
| CCR5-185 | − | GCUUGUGACACGGACUCAAG | 20 | 538 |
| CCR5-186 | − | CUUGUGACACGGACUCAAGU | 20 | 539 |
| CCR5-187 | − | UGACACGGACUCAAGUGGGC | 20 | 540 |
| CCR5-188 | − | CCCAGUCAGAGUUGUGCACA | 20 | 541 |
| CCR5-189 | − | CUUAGUUUUCAUACACAGCC | 20 | 542 |
| CCR5-190 | − | UUAGUUUUCAUACACAGCCU | 20 | 543 |
| CCR5-191 | − | UUUUCAUACACAGCCUGGGC | 20 | 544 |
| CCR5-192 | − | UUUCAUACACAGCCUGGGCU | 20 | 545 |
| CCR5-193 | − | UUCAUACACAGCCUGGGCUG | 20 | 546 |
| CCR5-194 | − | UCAUACACAGCCUGGGCUGG | 20 | 547 |
| CCR5-195 | − | UACACAGCCUGGGCUGGGGG | 20 | 548 |
| CCR5-196 | − | ACACAGCCUGGGCUGGGGGU | 20 | 549 |
| CCR5-197 | − | CACAGCCUGGGCUGGGGGUG | 20 | 550 |
| CCR5-198 | − | AGCCUGGGCUGGGGGUGGGG | 20 | 551 |
| CCR5-199 | − | GCCUGGGCUGGGGGUGGGGU | 20 | 552 |
| CCR5-200 | − | GGCUGGGGGUGGGGUGGGAG | 20 | 553 |
| CCR5-201 | − | UGGGAGAGGUCUUUUUUAAA | 20 | 554 |
| CCR5-202 | − | AAAGGAAGUUACUGUUAUAG | 20 | 555 |
| CCR5-203 | − | AAGGAAGUUACUGUUAUAGA | 20 | 556 |
| CCR5-204 | − | CUAAGAUUCAUCCAUUUAUU | 20 | 557 |
| CCR5-205 | − | ACAACUUUUUACCUAGUACA | 20 | 558 |
| CCR5-206 | − | CCUAGUACAAGGCAACAUAU | 20 | 559 |
| CCR5-207 | − | GUUGUAAAUGUGUUUAAAAC | 20 | 560 |
| CCR5-208 | − | AACAGGUCUUUGUCUUGCUA | 20 | 561 |
| CCR5-209 | − | ACAGGUCUUUGUCUUGCUAU | 20 | 562 |
| CCR5-210 | − | CAGGUCUUUGUCUUGCUAUG | 20 | 563 |
| CCR5-211 | − | CAUGUGUGAUUUCCCCUCCA | 20 | 564 |
| CCR5-212 | − | GUGAUUUCCCCUCCAAGGUA | 20 | 565 |
| CCR5-213 | − | AGUUUCACUGACUUAGAACC | 20 | 566 |
| CCR5-214 | − | AGAACCAGGCGAGAGACUUG | 20 | 567 |
| CCR5-215 | − | CAGGCGAGAGACUUGUGGCC | 20 | 568 |
| CCR5-216 | − | AGGCGAGAGACUUGUGGCCU | 20 | 569 |
| CCR5-217 | − | GACUUGUGGCCUGGGAGAGC | 20 | 570 |
| CCR5-218 | − | ACUUGUGGCCUGGGAGAGCU | 20 | 571 |
| CCR5-219 | − | CUUGUGGCCUGGGAGAGCUG | 20 | 572 |
| CCR5-220 | − | GGGAAGCUUCUUAAAUGAGA | 20 | 573 |
| CCR5-221 | − | AAAUGAGAAGGAAUUUGAGU | 20 | 574 |
| CCR5-222 | − | UGAGUUGGAUCAUCUAUUGC | 20 | 575 |
| CCR5-223 | − | GCCUCACUGCAAGCACUGCA | 20 | 576 |
| CCR5-224 | − | CCUCACUGCAAGCACUGCAU | 20 | 577 |
| CCR5-225 | − | AAGCACUGCAUGGGCAAGCU | 20 | 578 |
| CCR5-226 | − | UGGGCAAGCUUGGCUGUAGA | 20 | 579 |
| CCR5-227 | − | GCUGUAGAAGGAGACAGAGC | 20 | 580 |
| CCR5-228 | − | UAGAAGGAGACAGAGCUGGU | 20 | 581 |
| CCR5-229 | − | AGAAGGAGACAGAGCUGGUU | 20 | 582 |
| CCR5-230 | − | CAGAGCUGGUUGGGAAGACA | 20 | 583 |
| CCR5-231 | − | AGAGCUGGUUGGGAAGACAU | 20 | 584 |
| CCR5-232 | − | GAGCUGGUUGGGAAGACAUG | 20 | 585 |
| CCR5-233 | − | CUGGUUGGGAAGACAUGGGG | 20 | 586 |
| CCR5-234 | − | UUGGGAAGACAUGGGGAGGA | 20 | 587 |
| CCR5-235 | − | AGACAUGGGGAGGAAGGACA | 20 | 588 |
| CCR5-236 | − | UAGAUCAUGAAGAACCUUGA | 20 | 589 |
| CCR5-237 | − | GUCUAAGUCAUGAGCUGAGC | 20 | 590 |
| CCR5-238 | − | UCUAAGUCAUGAGCUGAGCA | 20 | 591 |
| CCR5-239 | − | UGAGCUGAGCAGGGAGAUCC | 20 | 592 |
| CCR5-240 | − | CUGAGCAGGGAGAUCCUGGU | 20 | 593 |
| CCR5-241 | − | AUCCUGGUUGGUGUUGCAGA | 20 | 594 |
| CCR5-242 | − | GUUGCAGAAGGUUUACUCUG | 20 | 595 |
| CCR5-243 | − | AAGGUUUACUCUGUGGCCAA | 20 | 596 |
| CCR5-244 | − | GUUUACUCUGUGGCCAAAGG | 20 | 597 |
| CCR5-245 | − | UUUACUCUGUGGCCAAAGGA | 20 | 598 |
| CCR5-246 | − | UCUGUGGCCAAAGGAGGGUC | 20 | 599 |
| CCR5-247 | − | UGGCCAAAGGAGGGUCAGGA | 20 | 600 |
| CCR5-248 | − | GUCAGGAAGGAUGAGCAUUU | 20 | 601 |
| CCR5-249 | − | UCAGGAAGGAUGAGCAUUUA | 20 | 602 |
| CCR5-250 | − | AAGGAUGAGCAUUUAGGGCA | 20 | 603 |
| CCR5-251 | − | GGAGACCACCAACAGCCCUC | 20 | 604 |
| CCR5-252 | − | CCACCAACAGCCCUCAGGUC | 20 | 605 |
| CCR5-253 | − | CACCAACAGCCCUCAGGUCA | 20 | 606 |
| CCR5-254 | − | ACAGCCCUCAGGUCAGGGUG | 20 | 607 |
| CCR5-255 | − | CCCUCAGGUCAGGGUGAGGA | 20 | 608 |
| CCR5-256 | − | GAUGGCCUCUGCUAAGCUCA | 20 | 609 |
| CCR5-257 | − | UCUGCUAAGCUCAAGGCGUG | 20 | 610 |
| CCR5-258 | − | CUAAGCUCAAGGCGUGAGGA | 20 | 611 |
| CCR5-259 | − | UAAGCUCAAGGCGUGAGGAU | 20 | 612 |
| CCR5-260 | − | CUCAAGGCGUGAGGAUGGGA | 20 | 613 |
| CCR5-261 | − | AAGGCGUGAGGAUGGGAAGG | 20 | 614 |
| CCR5-262 | − | AGGCGUGAGGAUGGGAAGGA | 20 | 615 |
| CCR5-263 | − | CGUGAGGAUGGGAAGGAGGG | 20 | 616 |
| CCR5-264 | − | GAAGGAGGGAGGUAUUCGUA | 20 | 617 |
| CCR5-265 | − | GAGGGAGGUAUUCGUAAGGA | 20 | 618 |
| CCR5-266 | − | AGGGAGGUAUUCGUAAGGAU | 20 | 619 |
| CCR5-267 | − | AGGUAUUCGUAAGGAUGGGA | 20 | 620 |
| CCR5-268 | − | UAUUCGUAAGGAUGGGAAGG | 20 | 621 |
| CCR5-269 | − | AUUCGUAAGGAUGGGAAGGA | 20 | 622 |
| CCR5-270 | − | CGUAAGGAUGGGAAGGAGGG | 20 | 623 |
| CCR5-271 | − | AGGUAUUCGUGCAGCAUAUG | 20 | 624 |
| CCR5-272 | − | GGAUGCAGAGUCAGCAGAAC | 20 | 625 |
| CCR5-273 | − | GAUGCAGAGUCAGCAGAACU | 20 | 626 |
| CCR5-274 | − | AUGCAGAGUCAGCAGAACUG | 20 | 627 |
| CCR5-275 | − | CAGAGUCAGCAGAACUGGGG | 20 | 628 |
| CCR5-276 | − | CAGCAGAACUGGGGUGGAUU | 20 | 629 |
| CCR5-277 | − | AGCAGAACUGGGGUGGAUUU | 20 | 630 |
| CCR5-278 | − | GAACUGGGGUGGAUUUGGGU | 20 | 631 |
| CCR5-279 | − | GUGGAUUUGGGUUGGAAGUG | 20 | 632 |
| CCR5-280 | − | UGGAUUUGGGUUGGAAGUGA | 20 | 633 |
| CCR5-281 | − | GUUGGAAGUGAGGGUCAGAG | 20 | 634 |
| CCR5-282 | − | UCCCUAGUCUUCAAGCAGAU | 20 | 635 |
| CCR5-283 | − | GAAAAGACAUCAAGCACAGA | 20 | 636 |
| CCR5-284 | − | AAGACAUCAAGCACAGAAGG | 20 | 637 |
| CCR5-285 | − | ACAUCAAGCACAGAAGGAGG | 20 | 638 |
| CCR5-286 | − | UCAAGCACAGAAGGAGGAGG | 20 | 639 |
| CCR5-287 | − | AGCACAGAAGGAGGAGGAGG | 20 | 640 |
| CCR5-288 | − | GAAGGAGGAGGAGGAGGUUU | 20 | 641 |
| CCR5-289 | − | GGUUUAGGUCAAGAAGAAGA | 20 | 642 |
| CCR5-290 | − | AGGUCAAGAAGAAGAUGGAU | 20 | 643 |
| CCR5-291 | − | AGAAGAUGGAUUGGUGUAAA | 20 | 644 |
| CCR5-292 | − | GAUGGAUUGGUGUAAAAGGA | 20 | 645 |
| CCR5-293 | − | AUGGAUUGGUGUAAAAGGAU | 20 | 646 |
| CCR5-294 | − | UUGGUGUAAAAGGAUGGGUC | 20 | 647 |
| CCR5-295 | − | CACAGUCUCACCCAGACUCC | 20 | 648 |
| CCR5-296 | − | CCAUCCCAGCUGAAAUACUG | 20 | 649 |
| CCR5-297 | − | CAUCCCAGCUGAAAUACUGA | 20 | 650 |
| CCR5-298 | − | AUCCCAGCUGAAAUACUGAG | 20 | 651 |
| CCR5-299 | − | UGAAAUACUGAGGGGUCUCC | 20 | 652 |
| CCR5-300 | − | AAUACUGAGGGGUCUCCAGG | 20 | 653 |
| CCR5-301 | − | ACUAGAUUUAUGAAUACACG | 20 | 654 |
| CCR5-302 | − | UUAUGAAUACACGAGGUAUG | 20 | 655 |
| CCR5-303 | − | AUACACGAGGUAUGAGGUCU | 20 | 656 |
| CCR5-304 | − | UCAGCUCACACAUGAGAUCU | 20 | 657 |
| CCR5-305 | − | UCACACAUGAGAUCUAGGUG | 20 | 658 |
| CCR5-306 | − | AUUACCUAGUAGUCAUUUCA | 20 | 659 |
| CCR5-307 | − | UUACCUAGUAGUCAUUUCAU | 20 | 660 |
| CCR5-308 | − | GUAGUCAUUUCAUGGGUUGU | 20 | 661 |
| CCR5-309 | − | UAGUCAUUUCAUGGGUUGUU | 20 | 662 |
| CCR5-310 | − | UCAUUUCAUGGGUUGUUGGG | 20 | 663 |
| CCR5-311 | − | GUUGUUGGGAGGAUUCUAUG | 20 | 664 |
| CCR5-312 | − | GGAUUCUAUGAGGCAACCAC | 20 | 665 |
| CCR5-313 | − | AAACUCUUAGUUACUCAUUC | 20 | 666 |
| CCR5-314 | − | AACUCUUAGUUACUCAUUCA | 20 | 667 |
| CCR5-315 | − | CUGAGCAAAGCAUUGAGCAA | 20 | 668 |
| CCR5-316 | − | UGAGCAAAGCAUUGAGCAAA | 20 | 669 |
| CCR5-317 | − | GAGCAAAGCAUUGAGCAAAG | 20 | 670 |
| CCR5-318 | − | UGAGCAAAGGGGUCCCAUAG | 20 | 671 |
| CCR5-319 | − | AAAGGGGUCCCAUAGAGGUG | 20 | 672 |
| CCR5-320 | − | AAGGGGUCCCAUAGAGGUGA | 20 | 673 |
| CCR5-321 | − | UGCCCAGUGCACACAAGUGU | 20 | 674 |
| CCR5-322 | − | UUCUGCAUUUAACCGUCAAU | 20 | 675 |
| CCR5-323 | − | AUUUAACCGUCAAUAGGCAA | 20 | 676 |
| CCR5-324 | − | UUUAACCGUCAAUAGGCAAA | 20 | 677 |
| CCR5-325 | − | UUAACCGUCAAUAGGCAAAG | 20 | 678 |
| CCR5-326 | − | UAACCGUCAAUAGGCAAAGG | 20 | 679 |
| CCR5-327 | − | AACCGUCAAUAGGCAAAGGG | 20 | 680 |
| CCR5-328 | − | GUCAAUAGGCAAAGGGGGGA | 20 | 681 |
| CCR5-329 | − | UCAAUAGGCAAAGGGGGGAA | 20 | 682 |
| CCR5-330 | − | GGGGAAGGGACAUAUUCAUU | 20 | 683 |
| CCR5-331 | − | CCUCCGUAUUUCAGACUGAA | 20 | 684 |
| CCR5-332 | − | CUCCGUAUUUCAGACUGAAU | 20 | 685 |
| CCR5-333 | − | UCCGUAUUUCAGACUGAAUG | 20 | 686 |
| CCR5-334 | − | CCGUAUUUCAGACUGAAUGG | 20 | 687 |
| CCR5-335 | − | UAUUUCAGACUGAAUGGGGG | 20 | 688 |
| CCR5-336 | − | AUUUCAGACUGAAUGGGGGU | 20 | 689 |
| CCR5-337 | − | UUUCAGACUGAAUGGGGGUG | 20 | 690 |
| CCR5-338 | − | UUCAGACUGAAUGGGGGUGG | 20 | 691 |
| CCR5-339 | − | UCAGACUGAAUGGGGGUGGG | 20 | 692 |
| CCR5-340 | − | CAGACUGAAUGGGGGUGGGG | 20 | 693 |
| CCR5-341 | − | AGACUGAAUGGGGGUGGGGG | 20 | 694 |
| CCR5-342 | − | GGGGGUGGGGGGGGCGCCUU | 20 | 695 |
| CCR5-343 | − | UGAAUAUACCCCUUAGUGUU | 20 | 696 |
| CCR5-344 | − | GAAUAUACCCCUUAGUGUUU | 20 | 697 |
| CCR5-345 | − | UUUGGGUAUAUUCAUUUCAA | 20 | 698 |
| CCR5-346 | − | UUGGGUAUAUUCAUUUCAAA | 20 | 699 |
| CCR5-347 | − | CAUUUCAAAGGGAGAGAGAG | 20 | 700 |
| CCR5-348 | − | ACUUGAGACUGUUUUGAAUU | 20 | 701 |
| CCR5-349 | − | CUUGAGACUGUUUUGAAUUU | 20 | 702 |
| CCR5-350 | − | UUGAGACUGUUUUGAAUUUG | 20 | 703 |
| CCR5-351 | − | UGAGACUGUUUUGAAUUUGG | 20 | 704 |
| CCR5-352 | − | ACUGUUUUGAAUUUGGGGGA | 20 | 705 |
| CCR5-353 | − | GGCUAAAACCAUCAUAGUAC | 20 | 706 |
| CCR5-354 | − | AAACCAUCAUAGUACAGGUA | 20 | 707 |
| CCR5-355 | − | AUCAUAGUACAGGUAAGGUG | 20 | 708 |
| CCR5-356 | − | UCAUAGUACAGGUAAGGUGA | 20 | 709 |
| CCR5-357 | − | UAAGGUGAGGGAAUAGUAAG | 20 | 710 |
| CCR5-358 | − | GUAAGUGGUGAGAACUACUC | 20 | 711 |
| CCR5-359 | − | UAAGUGGUGAGAACUACUCA | 20 | 712 |
| CCR5-360 | − | GAGAACUACUCAGGGAAUGA | 20 | 713 |
| CCR5-361 | − | GAAGGUGUCAGAAUAAUAAG | 20 | 714 |
| CCR5-362 | − | UCUCAGCCUCUGAAUAUGAA | 20 | 715 |
| CCR5-363 | − | AAUAUGAACGGUGAGCAUUG | 20 | 716 |
| CCR5-364 | − | UGAGCAUUGUGGCUGUCAGC | 20 | 717 |
| CCR5-365 | − | CUGUCAGCAGGAAGCAACGA | 20 | 718 |
| CCR5-366 | − | UGUCAGCAGGAAGCAACGAA | 20 | 719 |
| CCR5-367 | − | UUCCUUUUGCUCUUAAGUUG | 20 | 720 |
| CCR5-368 | − | GGAGAGUGCAACAGUAGCAU | 20 | 721 |
| CCR5-369 | − | UAGCAUAGGACCCUACCCUC | 20 | 722 |
| CCR5-370 | − | AGCAUAGGACCCUACCCUCU | 20 | 723 |
| CCR5-371 | − | ACAGUCAGUAUCAAUUC | 17 | 724 |
| CCR5-372 | − | AUUAAAGAUAGUCAUCU | 17 | 725 |
| CCR5-373 | − | UUAAAGAUAGUCAUCUU | 17 | 726 |
| CCR5-374 | − | UAAAGAUAGUCAUCUUG | 17 | 727 |
| CCR5-375 | − | GAUAGUCAUCUUGGGGC | 17 | 728 |
| CCR5-376 | − | CCUGCCGCUGCUUGUCA | 17 | 729 |
| CCR5-377 | − | CAUGGUCAUCUGCUACU | 17 | 730 |
| CCR5-378 | − | AUGGUCAUCUGCUACUC | 17 | 731 |
| CCR5-379 | − | UCCUAAAAACUCUGCUU | 17 | 732 |
| CCR5-380 | − | GUCGAAAUGAGAAGAAG | 17 | 733 |
| CCR5-381 | − | AUGAGAAGAAGAGGCAC | 17 | 734 |
| CCR5-382 | − | UGAGAAGAAGAGGCACA | 17 | 735 |
| CCR5-383 | − | AGAGGCACAGGGCUGUG | 17 | 736 |
| CCR5-384 | − | UUGUUUAUUUUCUCUUC | 17 | 737 |
| CCR5-385 | − | UGUUUAUUUUCUCUUCU | 17 | 738 |
| CCR5-386 | − | UCUCCUGAACACCUUCC | 17 | 739 |
| CCR5-387 | − | ACCUUCCAGGAAUUCUU | 17 | 740 |
| CCR5-388 | − | AUUGCAGUAGCUCUAAC | 17 | 741 |
| CCR5-389 | − | CAGUAGCUCUAACAGGU | 17 | 742 |
| CCR5-390 | − | GUUGGACCAAGCUAUGC | 17 | 743 |
| CCR5-391 | − | CAGGUGACAGAGACUCU | 17 | 744 |
| CCR5-392 | − | AGGUGACAGAGACUCUU | 17 | 745 |
| CCR5-393 | − | AUCAUCUAUGCCUUUGU | 17 | 746 |
| CCR5-394 | − | UCAUCUAUGCCUUUGUC | 17 | 747 |
| CCR5-395 | − | CAUCUAUGCCUUUGUCG | 17 | 748 |
| CCR5-396 | − | UUCUAUUUUCCAGCAAG | 17 | 749 |
| CCR5-397 | − | GUUUACACCCGAUCCAC | 17 | 750 |
| CCR5-398 | − | UUUACACCCGAUCCACU | 17 | 751 |
| CCR5-399 | − | UUACACCCGAUCCACUG | 17 | 752 |
| CCR5-400 | − | CCGAUCCACUGGGGAGC | 17 | 753 |
| CCR5-401 | − | GGAGCAGGAAAUAUCUG | 17 | 754 |
| CCR5-402 | − | GAGCAGGAAAUAUCUGU | 17 | 755 |
| CCR5-403 | − | UCUGUGGGCUUGUGACA | 17 | 756 |
| CCR5-404 | − | UGUGACACGGACUCAAG | 17 | 757 |
| CCR5-405 | − | GUGACACGGACUCAAGU | 17 | 758 |
| CCR5-406 | − | CACGGACUCAAGUGGGC | 17 | 759 |
| CCR5-407 | − | AGUCAGAGUUGUGCACA | 17 | 760 |
| CCR5-408 | − | AGUUUUCAUACACAGCC | 17 | 761 |
| CCR5-409 | − | GUUUUCAUACACAGCCU | 17 | 762 |
| CCR5-410 | − | UCAUACACAGCCUGGGC | 17 | 763 |
| CCR5-411 | − | CAUACACAGCCUGGGCU | 17 | 764 |
| CCR5-412 | − | AUACACAGCCUGGGCUG | 17 | 765 |
| CCR5-413 | − | UACACAGCCUGGGCUGG | 17 | 766 |
| CCR5-414 | − | ACAGCCUGGGCUGGGGG | 17 | 767 |
| CCR5-415 | − | CAGCCUGGGCUGGGGGU | 17 | 768 |
| CCR5-416 | − | AGCCUGGGCUGGGGGUG | 17 | 769 |
| CCR5-417 | − | CUGGGCUGGGGGUGGGG | 17 | 770 |
| CCR5-418 | − | UGGGCUGGGGGUGGGGU | 17 | 771 |
| CCR5-419 | − | UGGGGGUGGGGUGGGAG | 17 | 772 |
| CCR5-420 | − | GAGAGGUCUUUUUUAAA | 17 | 773 |
| CCR5-421 | − | GGAAGUUACUGUUAUAG | 17 | 774 |
| CCR5-422 | − | GAAGUUACUGUUAUAGA | 17 | 775 |
| CCR5-423 | − | AGAUUCAUCCAUUUAUU | 17 | 776 |
| CCR5-424 | − | ACUUUUUACCUAGUACA | 17 | 777 |
| CCR5-425 | − | AGUACAAGGCAACAUAU | 17 | 778 |
| CCR5-426 | − | GUAAAUGUGUUUAAAAC | 17 | 779 |
| CCR5-427 | − | AGGUCUUUGUCUUGCUA | 17 | 780 |
| CCR5-428 | − | GGUCUUUGUCUUGCUAU | 17 | 781 |
| CCR5-429 | − | GUCUUUGUCUUGCUAUG | 17 | 782 |
| CCR5-430 | − | GUGUGAUUUCCCCUCCA | 17 | 783 |
| CCR5-431 | − | AUUUCCCCUCCAAGGUA | 17 | 784 |
| CCR5-432 | − | UUCACUGACUUAGAACC | 17 | 785 |
| CCR5-433 | − | ACCAGGCGAGAGACUUG | 17 | 786 |
| CCR5-434 | − | GCGAGAGACUUGUGGCC | 17 | 787 |
| CCR5-435 | − | CGAGAGACUUGUGGCCU | 17 | 788 |
| CCR5-436 | − | UUGUGGCCUGGGAGAGC | 17 | 789 |
| CCR5-437 | − | UGUGGCCUGGGAGAGCU | 17 | 790 |
| CCR5-438 | − | GUGGCCUGGGAGAGCUG | 17 | 791 |
| CCR5-439 | − | AAGCUUCUUAAAUGAGA | 17 | 792 |
| CCR5-440 | − | UGAGAAGGAAUUUGAGU | 17 | 793 |
| CCR5-441 | − | GUUGGAUCAUCUAUUGC | 17 | 794 |
| CCR5-442 | − | UCACUGCAAGCACUGCA | 17 | 795 |
| CCR5-443 | − | CACUGCAAGCACUGCAU | 17 | 796 |
| CCR5-444 | − | CACUGCAUGGGCAAGCU | 17 | 797 |
| CCR5-445 | − | GCAAGCUUGGCUGUAGA | 17 | 798 |
| CCR5-446 | − | GUAGAAGGAGACAGAGC | 17 | 799 |
| CCR5-447 | − | AAGGAGACAGAGCUGGU | 17 | 800 |
| CCR5-448 | − | AGGAGACAGAGCUGGUU | 17 | 801 |
| CCR5-449 | − | AGCUGGUUGGGAAGACA | 17 | 802 |
| CCR5-450 | − | GCUGGUUGGGAAGACAU | 17 | 803 |
| CCR5-451 | − | CUGGUUGGGAAGACAUG | 17 | 804 |
| CCR5-452 | − | GUUGGGAAGACAUGGGG | 17 | 805 |
| CCR5-453 | − | GGAAGACAUGGGGAGGA | 17 | 806 |
| CCR5-454 | − | CAUGGGGAGGAAGGACA | 17 | 807 |
| CCR5-455 | − | AUCAUGAAGAACCUUGA | 17 | 808 |
| CCR5-456 | − | UAAGUCAUGAGCUGAGC | 17 | 809 |
| CCR5-457 | − | AAGUCAUGAGCUGAGCA | 17 | 810 |
| CCR5-458 | − | GCUGAGCAGGGAGAUCC | 17 | 811 |
| CCR5-459 | − | AGCAGGGAGAUCCUGGU | 17 | 812 |
| CCR5-460 | − | CUGGUUGGUGUUGCAGA | 17 | 813 |
| CCR5-461 | − | GCAGAAGGUUUACUCUG | 17 | 814 |
| CCR5-462 | − | GUUUACUCUGUGGCCAA | 17 | 815 |
| CCR5-463 | − | UACUCUGUGGCCAAAGG | 17 | 816 |
| CCR5-464 | − | ACUCUGUGGCCAAAGGA | 17 | 817 |
| CCR5-465 | − | GUGGCCAAAGGAGGGUC | 17 | 818 |
| CCR5-466 | − | CCAAAGGAGGGUCAGGA | 17 | 819 |
| CCR5-467 | − | AGGAAGGAUGAGCAUUU | 17 | 820 |
| CCR5-468 | − | GGAAGGAUGAGCAUUUA | 17 | 821 |
| CCR5-469 | − | GAUGAGCAUUUAGGGCA | 17 | 822 |
| CCR5-470 | − | GACCACCAACAGCCCUC | 17 | 823 |
| CCR5-471 | − | CCAACAGCCCUCAGGUC | 17 | 824 |
| CCR5-472 | − | CAACAGCCCUCAGGUCA | 17 | 825 |
| CCR5-473 | − | GCCCUCAGGUCAGGGUG | 17 | 826 |
| CCR5-474 | − | UCAGGUCAGGGUGAGGA | 17 | 827 |
| CCR5-475 | − | GGCCUCUGCUAAGCUCA | 17 | 828 |
| CCR5-476 | − | GCUAAGCUCAAGGCGUG | 17 | 829 |
| CCR5-477 | − | AGCUCAAGGCGUGAGGA | 17 | 830 |
| CCR5-478 | − | GCUCAAGGCGUGAGGAU | 17 | 831 |
| CCR5-479 | − | AAGGCGUGAGGAUGGGA | 17 | 832 |
| CCR5-480 | − | GCGUGAGGAUGGGAAGG | 17 | 833 |
| CCR5-481 | − | CGUGAGGAUGGGAAGGA | 17 | 834 |
| CCR5-482 | − | GAGGAUGGGAAGGAGGG | 17 | 835 |
| CCR5-483 | − | GGAGGGAGGUAUUCGUA | 17 | 836 |
| CCR5-484 | − | GGAGGUAUUCGUAAGGA | 17 | 837 |
| CCR5-485 | − | GAGGUAUUCGUAAGGAU | 17 | 838 |
| CCR5-486 | − | UAUUCGUAAGGAUGGGA | 17 | 839 |
| CCR5-487 | − | UCGUAAGGAUGGGAAGG | 17 | 840 |
| CCR5-488 | − | CGUAAGGAUGGGAAGGA | 17 | 841 |
| CCR5-489 | − | AAGGAUGGGAAGGAGGG | 17 | 842 |
| CCR5-490 | − | UAUUCGUGCAGCAUAUG | 17 | 843 |
| CCR5-491 | − | UGCAGAGUCAGCAGAAC | 17 | 844 |
| CCR5-492 | − | GCAGAGUCAGCAGAACU | 17 | 845 |
| CCR5-493 | − | CAGAGUCAGCAGAACUG | 17 | 846 |
| CCR5-494 | − | AGUCAGCAGAACUGGGG | 17 | 847 |
| CCR5-495 | − | CAGAACUGGGGUGGAUU | 17 | 848 |
| CCR5-496 | − | AGAACUGGGGUGGAUUU | 17 | 849 |
| CCR5-497 | − | CUGGGGUGGAUUUGGGU | 17 | 850 |
| CCR5-498 | − | GAUUUGGGUUGGAAGUG | 17 | 851 |
| CCR5-499 | − | AUUUGGGUUGGAAGUGA | 17 | 852 |
| CCR5-500 | − | GGAAGUGAGGGUCAGAG | 17 | 853 |
| CCR5-501 | − | CUAGUCUUCAAGCAGAU | 17 | 854 |
| CCR5-502 | − | AAGACAUCAAGCACAGA | 17 | 855 |
| CCR5-503 | − | ACAUCAAGCACAGAAGG | 17 | 856 |
| CCR5-504 | − | UCAAGCACAGAAGGAGG | 17 | 857 |
| CCR5-505 | − | AGCACAGAAGGAGGAGG | 17 | 858 |
| CCR5-506 | − | ACAGAAGGAGGAGGAGG | 17 | 859 |
| CCR5-507 | − | GGAGGAGGAGGAGGUUU | 17 | 860 |
| CCR5-508 | − | UUAGGUCAAGAAGAAGA | 17 | 861 |
| CCR5-509 | − | UCAAGAAGAAGAUGGAU | 17 | 862 |
| CCR5-510 | − | AGAUGGAUUGGUGUAAA | 17 | 863 |
| CCR5-511 | − | GGAUUGGUGUAAAAGGA | 17 | 864 |
| CCR5-512 | − | GAUUGGUGUAAAAGGAU | 17 | 865 |
| CCR5-513 | − | GUGUAAAAGGAUGGGUC | 17 | 866 |
| CCR5-514 | − | AGUCUCACCCAGACUCC | 17 | 867 |
| CCR5-515 | − | UCCCAGCUGAAAUACUG | 17 | 868 |
| CCR5-516 | − | CCCAGCUGAAAUACUGA | 17 | 869 |
| CCR5-517 | − | CCAGCUGAAAUACUGAG | 17 | 870 |
| CCR5-518 | − | AAUACUGAGGGGUCUCC | 17 | 871 |
| CCR5-519 | − | ACUGAGGGGUCUCCAGG | 17 | 872 |
| CCR5-520 | − | AGAUUUAUGAAUACACG | 17 | 873 |
| CCR5-521 | − | UGAAUACACGAGGUAUG | 17 | 874 |
| CCR5-522 | − | CACGAGGUAUGAGGUCU | 17 | 875 |
| CCR5-523 | − | GCUCACACAUGAGAUCU | 17 | 876 |
| CCR5-524 | − | CACAUGAGAUCUAGGUG | 17 | 877 |
| CCR5-525 | − | ACCUAGUAGUCAUUUCA | 17 | 878 |
| CCR5-526 | − | CCUAGUAGUCAUUUCAU | 17 | 879 |
| CCR5-527 | − | GUCAUUUCAUGGGUUGU | 17 | 880 |
| CCR5-528 | − | UCAUUUCAUGGGUUGUU | 17 | 881 |
| CCR5-529 | − | UUUCAUGGGUUGUUGGG | 17 | 882 |
| CCR5-530 | − | GUUGGGAGGAUUCUAUG | 17 | 883 |
| CCR5-531 | − | UUCUAUGAGGCAACCAC | 17 | 884 |
| CCR5-532 | − | CUCUUAGUUACUCAUUC | 17 | 885 |
| CCR5-533 | − | UCUUAGUUACUCAUUCA | 17 | 886 |
| CCR5-534 | − | AGCAAAGCAUUGAGCAA | 17 | 887 |
| CCR5-535 | − | GCAAAGCAUUGAGCAAA | 17 | 888 |
| CCR5-536 | − | CAAAGCAUUGAGCAAAG | 17 | 889 |
| CCR5-537 | − | GCAAAGGGGUCCCAUAG | 17 | 890 |
| CCR5-538 | − | GGGGUCCCAUAGAGGUG | 17 | 891 |
| CCR5-539 | − | GGGUCCCAUAGAGGUGA | 17 | 892 |
| CCR5-540 | − | CCAGUGCACACAAGUGU | 17 | 893 |
| CCR5-541 | − | UGCAUUUAACCGUCAAU | 17 | 894 |
| CCR5-542 | − | UAACCGUCAAUAGGCAA | 17 | 895 |
| CCR5-543 | − | AACCGUCAAUAGGCAAA | 17 | 896 |
| CCR5-544 | − | ACCGUCAAUAGGCAAAG | 17 | 897 |
| CCR5-545 | − | CCGUCAAUAGGCAAAGG | 17 | 898 |
| CCR5-546 | − | CGUCAAUAGGCAAAGGG | 17 | 899 |
| CCR5-547 | − | AAUAGGCAAAGGGGGGA | 17 | 900 |
| CCR5-548 | − | AUAGGCAAAGGGGGGAA | 17 | 901 |
| CCR5-549 | − | GAAGGGACAUAUUCAUU | 17 | 902 |
| CCR5-550 | − | CCGUAUUUCAGACUGAA | 17 | 903 |
| CCR5-551 | − | CGUAUUUCAGACUGAAU | 17 | 904 |
| CCR5-552 | − | GUAUUUCAGACUGAAUG | 17 | 905 |
| CCR5-553 | − | UAUUUCAGACUGAAUGG | 17 | 906 |
| CCR5-554 | − | UUCAGACUGAAUGGGGG | 17 | 907 |
| CCR5-555 | − | UCAGACUGAAUGGGGGU | 17 | 908 |
| CCR5-556 | − | CAGACUGAAUGGGGGUG | 17 | 909 |
| CCR5-557 | − | AGACUGAAUGGGGGUGG | 17 | 910 |
| CCR5-558 | − | GACUGAAUGGGGGUGGG | 17 | 911 |
| CCR5-559 | − | ACUGAAUGGGGGUGGGG | 17 | 912 |
| CCR5-560 | − | CUGAAUGGGGGUGGGGG | 17 | 913 |
| CCR5-561 | − | GGUGGGGGGGGCGCCUU | 17 | 914 |
| CCR5-562 | − | AUAUACCCCUUAGUGUU | 17 | 915 |
| CCR5-563 | − | UAUACCCCUUAGUGUUU | 17 | 916 |
| CCR5-564 | − | GGGUAUAUUCAUUUCAA | 17 | 917 |
| CCR5-565 | − | GGUAUAUUCAUUUCAAA | 17 | 918 |
| CCR5-566 | − | UUCAAAGGGAGAGAGAG | 17 | 919 |
| CCR5-567 | − | UGAGACUGUUUUGAAUU | 17 | 920 |
| CCR5-568 | − | GAGACUGUUUUGAAUUU | 17 | 921 |
| CCR5-569 | − | AGACUGUUUUGAAUUUG | 17 | 922 |
| CCR5-570 | − | GACUGUUUUGAAUUUGG | 17 | 923 |
| CCR5-571 | − | GUUUUGAAUUUGGGGGA | 17 | 924 |
| CCR5-572 | − | UAAAACCAUCAUAGUAC | 17 | 925 |
| CCR5-573 | − | CCAUCAUAGUACAGGUA | 17 | 926 |
| CCR5-574 | − | AUAGUACAGGUAAGGUG | 17 | 927 |
| CCR5-575 | − | UAGUACAGGUAAGGUGA | 17 | 928 |
| CCR5-576 | − | GGUGAGGGAAUAGUAAG | 17 | 929 |
| CCR5-577 | − | AGUGGUGAGAACUACUC | 17 | 930 |
| CCR5-578 | − | GUGGUGAGAACUACUCA | 17 | 931 |
| CCR5-579 | − | AACUACUCAGGGAAUGA | 17 | 932 |
| CCR5-580 | − | GGUGUCAGAAUAAUAAG | 17 | 933 |
| CCR5-581 | − | CAGCCUCUGAAUAUGAA | 17 | 934 |
| CCR5-582 | − | AUGAACGGUGAGCAUUG | 17 | 935 |
| CCR5-583 | − | GCAUUGUGGCUGUCAGC | 17 | 936 |
| CCR5-584 | − | UCAGCAGGAAGCAACGA | 17 | 937 |
| CCR5-585 | − | CAGCAGGAAGCAACGAA | 17 | 938 |
| CCR5-586 | − | CUUUUGCUCUUAAGUUG | 17 | 939 |
| CCR5-587 | − | GAGUGCAACAGUAGCAU | 17 | 940 |
| CCR5-588 | − | CAUAGGACCCUACCCUC | 17 | 941 |
| CCR5-589 | − | AUAGGACCCUACCCUCU | 17 | 942 |
| CCR5-590 | + | AUGUCAGAAUGUCUUUGACU | 20 | 943 |
| CCR5-591 | + | AUGUCUUUGACUUGGCCCAG | 20 | 944 |
| CCR5-592 | + | UGUCUUUGACUUGGCCCAGA | 20 | 945 |
| CCR5-593 | + | UUUGACUUGGCCCAGAGGGU | 20 | 946 |
| CCR5-594 | + | UUGACUUGGCCCAGAGGGUA | 20 | 947 |
| CCR5-595 | + | CUCCACAACUUAAGAGCAAA | 20 | 948 |
| CCR5-596 | + | UGCUCACCGUUCAUAUUCAG | 20 | 949 |
| CCR5-597 | + | UCACCUUACCUGUACUAUGA | 20 | 950 |
| CCR5-598 | + | AUGAAUAUACCCAAACACUA | 20 | 951 |
| CCR5-599 | + | UGAAUAUACCCAAACACUAA | 20 | 952 |
| CCR5-600 | + | GAAUAUACCCAAACACUAAG | 20 | 953 |
| CCR5-601 | + | AAGGGGUAUAUUCAUUUCAA | 20 | 954 |
| CCR5-602 | + | AGGGGUAUAUUCAUUUCAAA | 20 | 955 |
| CCR5-603 | + | GGUAUAUUCAUUUCAAAGGG | 20 | 956 |
| CCR5-604 | + | GUAUAUUCAUUUCAAAGGGA | 20 | 957 |
| CCR5-605 | + | ACGAUUUUUUCUGUUGCUUC | 20 | 958 |
| CCR5-606 | + | UCUGUUGCUUCUGGUUUGUC | 20 | 959 |
| CCR5-607 | + | GCUUCUGGUUUGUCUGGAGA | 20 | 960 |
| CCR5-608 | + | GUUUGUCUGGAGAAGGCAUC | 20 | 961 |
| CCR5-609 | + | GCAUCUGGAAUAAGUACCUA | 20 | 962 |
| CCR5-610 | + | CCCCCAUUCAGUCUGAAAUA | 20 | 963 |
| CCR5-611 | + | CCAUUCAGUCUGAAAUACGG | 20 | 964 |
| CCR5-612 | + | UCAGUCUGAAAUACGGAGGC | 20 | 965 |
| CCR5-613 | + | GCUGGUAAAUUGUACUUUUG | 20 | 966 |
| CCR5-614 | + | CUGGUAAAUUGUACUUUUGU | 20 | 967 |
| CCR5-615 | + | UUGUACUUUUGUGGGUUUUA | 20 | 968 |
| CCR5-616 | + | UUUGUGGGUUUUAAGGCUCA | 20 | 969 |
| CCR5-617 | + | UUCCCCCCUUUGCCUAUUGA | 20 | 970 |
| CCR5-618 | + | AUACCUACACUUGUGUGCAC | 20 | 971 |
| CCR5-619 | + | UACCUACACUUGUGUGCACU | 20 | 972 |
| CCR5-620 | + | UACACUUGUGUGCACUGGGC | 20 | 973 |
| CCR5-621 | + | AGGCAGCAUCUUAGUUUUUC | 20 | 974 |
| CCR5-622 | + | UCAGGCUUCCCUCACCUCUA | 20 | 975 |
| CCR5-623 | + | CAGGCUUCCCUCACCUCUAU | 20 | 976 |
| CCR5-624 | + | UAUGUGCUAAAUGCUGCCUG | 20 | 977 |
| CCR5-625 | + | CAACCCAUGAAAUGACUACU | 20 | 978 |
| CCR5-626 | + | UCAUAAAUCUAGUCUCCUCC | 20 | 979 |
| CCR5-627 | + | AGACCCCUCAGUAUUUCAGC | 20 | 980 |
| CCR5-628 | + | GACCCCUCAGUAUUUCAGCU | 20 | 981 |
| CCR5-629 | + | CCUCAGUAUUUCAGCUGGGA | 20 | 982 |
| CCR5-630 | + | CUCAGUAUUUCAGCUGGGAU | 20 | 983 |
| CCR5-631 | + | GUAUUUCAGCUGGGAUGGGA | 20 | 984 |
| CCR5-632 | + | GCAUUCAGUGAAAGACAGCC | 20 | 985 |
| CCR5-633 | + | GUGAAAGACAGCCUGGAGUC | 20 | 986 |
| CCR5-634 | + | UGAAAGACAGCCUGGAGUCU | 20 | 987 |
| CCR5-635 | + | CUGUGCUUGAUGUCUUUUCA | 20 | 988 |
| CCR5-636 | + | UGUGCUUGAUGUCUUUUCAA | 20 | 989 |
| CCR5-637 | + | CUCCAAUCUGCUUGAAGACU | 20 | 990 |
| CCR5-638 | + | UCCAAUCUGCUUGAAGACUA | 20 | 991 |
| CCR5-639 | + | UCACGCCUUGAGCUUAGCAG | 20 | 992 |
| CCR5-640 | + | GCCAUCCUCACCCUGACCUG | 20 | 993 |
| CCR5-641 | + | CCAUCCUCACCCUGACCUGA | 20 | 994 |
| CCR5-642 | + | CACCCUGACCUGAGGGCUGU | 20 | 995 |
| CCR5-643 | + | CCUGACCUGAGGGCUGUUGG | 20 | 996 |
| CCR5-644 | + | CAUCCUUCCUGACCCUCCUU | 20 | 997 |
| CCR5-645 | + | AACCUUCUGCAACACCAACC | 20 | 998 |
| CCR5-646 | + | UGCUCAGCUCAUGACUUAGA | 20 | 999 |
| CCR5-647 | + | UAGACGGAGCAAUGCCGUCA | 20 | 1000 |
| CCR5-648 | + | CCCAUGCAGUGCUUGCAGUG | 20 | 1001 |
| CCR5-649 | + | GAAGCUUCCCCAGCUCUCCC | 20 | 1002 |
| CCR5-650 | + | CAGGCCACAAGUCUCUCGCC | 20 | 1003 |
| CCR5-651 | + | GAAACUUAUUAACCAUACCU | 20 | 1004 |
| CCR5-652 | + | ACUUAUUAACCAUACCUUGG | 20 | 1005 |
| CCR5-653 | + | CUUAUUAACCAUACCUUGGA | 20 | 1006 |
| CCR5-654 | + | UUAUUAACCAUACCUUGGAG | 20 | 1007 |
| CCR5-655 | + | CCUAUAUGUUGCCUUGUACU | 20 | 1008 |
| CCR5-656 | + | GUACAUUUCUGAAAUAAUUU | 20 | 1009 |
| CCR5-657 | + | CAAGAAUCAGCAAUUCUCUG | 20 | 1010 |
| CCR5-658 | + | CUUUCUUUUAAAUAUACAUA | 20 | 1011 |
| CCR5-659 | + | AAAUAUACAUAAGGAACUUU | 20 | 1012 |
| CCR5-660 | + | AUAAGGAACUUUCGGAGUGA | 20 | 1013 |
| CCR5-661 | + | UAAGGAACUUUCGGAGUGAA | 20 | 1014 |
| CCR5-662 | + | CAAUAACUUGAUGCAUGUGA | 20 | 1015 |
| CCR5-663 | + | AAUAACUUGAUGCAUGUGAA | 20 | 1016 |
| CCR5-664 | + | AUAACUUGAUGCAUGUGAAG | 20 | 1017 |
| CCR5-665 | + | CAUGUGAAGGGGAGAUAAAA | 20 | 1018 |
| CCR5-666 | + | UUCAUCAACAUAUUUUGAUU | 20 | 1019 |
| CCR5-667 | + | AUUUGGCUUUCUAUAAUUGA | 20 | 1020 |
| CCR5-668 | + | UUUGGCUUUCUAUAAUUGAU | 20 | 1021 |
| CCR5-669 | + | UUAAACAGAUGCCAAAUAAA | 20 | 1022 |
| CCR5-670 | + | UCCCACCCCACCCCCAGCCC | 20 | 1023 |
| CCR5-671 | + | GCCAUGUGCACAACUCUGAC | 20 | 1024 |
| CCR5-672 | + | CCAUGUGCACAACUCUGACU | 20 | 1025 |
| CCR5-673 | + | AGAUAUUUCCUGCUCCCCAG | 20 | 1026 |
| CCR5-674 | + | UUUCCUGCUCCCCAGUGGAU | 20 | 1027 |
| CCR5-675 | + | UUCCUGCUCCCCAGUGGAUC | 20 | 1028 |
| CCR5-676 | + | GUAAACUGAGCUUGCUCGCU | 20 | 1029 |
| CCR5-677 | + | UAAACUGAGCUUGCUCGCUC | 20 | 1030 |
| CCR5-678 | + | CUCGCUCGGGAGCCUCUUGC | 20 | 1031 |
| CCR5-679 | + | ACAGCAUUUGCAGAAGCGUU | 20 | 1032 |
| CCR5-680 | + | AGCGUUUGGCAAUGUGCUUU | 20 | 1033 |
| CCR5-681 | + | GCUUUUGGAAGAAGACUAAG | 20 | 1034 |
| CCR5-682 | + | UCUGAACUUCUCCCCGACAA | 20 | 1035 |
| CCR5-683 | + | CCCGACAAAGGCAUAGAUGA | 20 | 1036 |
| CCR5-684 | + | CCGACAAAGGCAUAGAUGAU | 20 | 1037 |
| CCR5-685 | + | CGACAAAGGCAUAGAUGAUG | 20 | 1038 |
| CCR5-686 | + | UCUCUGUCACCUGCAUAGCU | 20 | 1039 |
| CCR5-687 | + | UAGAGCUACUGCAAUUAUUC | 20 | 1040 |
| CCR5-688 | + | UAUUCAGGCCAAAGAAUUCC | 20 | 1041 |
| CCR5-689 | + | CAGGCCAAAGAAUUCCUGGA | 20 | 1042 |
| CCR5-690 | + | AGAAUUCCUGGAAGGUGUUC | 20 | 1043 |
| CCR5-691 | + | CCUGGAAGGUGUUCAGGAGA | 20 | 1044 |
| CCR5-692 | + | CAGGAGAAGGACAAUGUUGU | 20 | 1045 |
| CCR5-693 | + | AGGAGAAGGACAAUGUUGUA | 20 | 1046 |
| CCR5-694 | + | GAGAAAAUAAACAAUCAUGA | 20 | 1047 |
| CCR5-695 | + | GACACCGAAGCAGAGUUUUU | 20 | 1048 |
| CCR5-696 | + | CAGAUGACCAUGACAAGCAG | 20 | 1049 |
| CCR5-697 | + | UGACCAUGACAAGCAGCGGC | 20 | 1050 |
| CCR5-698 | + | AGAUGACUAUCUUUAAUGUC | 20 | 1051 |
| CCR5-699 | + | CAGAAUUGAUACUGACUGUA | 20 | 1052 |
| CCR5-700 | + | GUAUGGAAAAUGAGAGCUGC | 20 | 1053 |
| CCR5-701 | + | UCAGAAUGUCUUUGACU | 17 | 1054 |
| CCR5-702 | + | UCUUUGACUUGGCCCAG | 17 | 1055 |
| CCR5-703 | + | CUUUGACUUGGCCCAGA | 17 | 1056 |
| CCR5-704 | + | GACUUGGCCCAGAGGGU | 17 | 1057 |
| CCR5-705 | + | ACUUGGCCCAGAGGGUA | 17 | 1058 |
| CCR5-706 | + | CACAACUUAAGAGCAAA | 17 | 1059 |
| CCR5-707 | + | UCACCGUUCAUAUUCAG | 17 | 1060 |
| CCR5-708 | + | CCUUACCUGUACUAUGA | 17 | 1061 |
| CCR5-709 | + | AAUAUACCCAAACACUA | 17 | 1062 |
| CCR5-710 | + | AUAUACCCAAACACUAA | 17 | 1063 |
| CCR5-711 | + | UAUACCCAAACACUAAG | 17 | 1064 |
| CCR5-712 | + | GGGUAUAUUCAUUUCAA | 17 | 1065 |
| CCR5-713 | + | GGUAUAUUCAUUUCAAA | 17 | 1066 |
| CCR5-714 | + | AUAUUCAUUUCAAAGGG | 17 | 1067 |
| CCR5-715 | + | UAUUCAUUUCAAAGGGA | 17 | 1068 |
| CCR5-716 | + | AUUUUUUCUGUUGCUUC | 17 | 1069 |
| CCR5-717 | + | GUUGCUUCUGGUUUGUC | 17 | 1070 |
| CCR5-718 | + | UCUGGUUUGUCUGGAGA | 17 | 1071 |
| CCR5-719 | + | UGUCUGGAGAAGGCAUC | 17 | 1072 |
| CCR5-720 | + | UCUGGAAUAAGUACCUA | 17 | 1073 |
| CCR5-721 | + | CCAUUCAGUCUGAAAUA | 17 | 1074 |
| CCR5-722 | + | UUCAGUCUGAAAUACGG | 17 | 1075 |
| CCR5-723 | + | GUCUGAAAUACGGAGGC | 17 | 1076 |
| CCR5-724 | + | GGUAAAUUGUACUUUUG | 17 | 1077 |
| CCR5-725 | + | GUAAAUUGUACUUUUGU | 17 | 1078 |
| CCR5-726 | + | UACUUUUGUGGGUUUUA | 17 | 1079 |
| CCR5-727 | + | GUGGGUUUUAAGGCUCA | 17 | 1080 |
| CCR5-728 | + | CCCCCUUUGCCUAUUGA | 17 | 1081 |
| CCR5-729 | + | CCUACACUUGUGUGCAC | 17 | 1082 |
| CCR5-730 | + | CUACACUUGUGUGCACU | 17 | 1083 |
| CCR5-731 | + | ACUUGUGUGCACUGGGC | 17 | 1084 |
| CCR5-732 | + | CAGCAUCUUAGUUUUUC | 17 | 1085 |
| CCR5-733 | + | GGCUUCCCUCACCUCUA | 17 | 1086 |
| CCR5-734 | + | GCUUCCCUCACCUCUAU | 17 | 1087 |
| CCR5-735 | + | GUGCUAAAUGCUGCCUG | 17 | 1088 |
| CCR5-736 | + | CCCAUGAAAUGACUACU | 17 | 1089 |
| CCR5-737 | + | UAAAUCUAGUCUCCUCC | 17 | 1090 |
| CCR5-738 | + | CCCCUCAGUAUUUCAGC | 17 | 1091 |
| CCR5-739 | + | CCCUCAGUAUUUCAGCU | 17 | 1092 |
| CCR5-740 | + | CAGUAUUUCAGCUGGGA | 17 | 1093 |
| CCR5-741 | + | AGUAUUUCAGCUGGGAU | 17 | 1094 |
| CCR5-742 | + | UUUCAGCUGGGAUGGGA | 17 | 1095 |
| CCR5-743 | + | UUCAGUGAAAGACAGCC | 17 | 1096 |
| CCR5-744 | + | AAAGACAGCCUGGAGUC | 17 | 1097 |
| CCR5-745 | + | AAGACAGCCUGGAGUCU | 17 | 1098 |
| CCR5-746 | + | UGCUUGAUGUCUUUUCA | 17 | 1099 |
| CCR5-747 | + | GCUUGAUGUCUUUUCAA | 17 | 1100 |
| CCR5-748 | + | CAAUCUGCUUGAAGACU | 17 | 1101 |
| CCR5-749 | + | AAUCUGCUUGAAGACUA | 17 | 1102 |
| CCR5-750 | + | CGCCUUGAGCUUAGCAG | 17 | 1103 |
| CCR5-751 | + | AUCCUCACCCUGACCUG | 17 | 1104 |
| CCR5-752 | + | UCCUCACCCUGACCUGA | 17 | 1105 |
| CCR5-753 | + | CCUGACCUGAGGGCUGU | 17 | 1106 |
| CCR5-754 | + | GACCUGAGGGCUGUUGG | 17 | 1107 |
| CCR5-755 | + | CCUUCCUGACCCUCCUU | 17 | 1108 |
| CCR5-756 | + | CUUCUGCAACACCAACC | 17 | 1109 |
| CCR5-757 | + | UCAGCUCAUGACUUAGA | 17 | 1110 |
| CCR5-758 | + | ACGGAGCAAUGCCGUCA | 17 | 1111 |
| CCR5-759 | + | AUGCAGUGCUUGCAGUG | 17 | 1112 |
| CCR5-760 | + | GCUUCCCCAGCUCUCCC | 17 | 1113 |
| CCR5-761 | + | GCCACAAGUCUCUCGCC | 17 | 1114 |
| CCR5-762 | + | ACUUAUUAACCAUACCU | 17 | 1115 |
| CCR5-763 | + | UAUUAACCAUACCUUGG | 17 | 1116 |
| CCR5-764 | + | AUUAACCAUACCUUGGA | 17 | 1117 |
| CCR5-765 | + | UUAACCAUACCUUGGAG | 17 | 1118 |
| CCR5-766 | + | AUAUGUUGCCUUGUACU | 17 | 1119 |
| CCR5-767 | + | CAUUUCUGAAAUAAUUU | 17 | 1120 |
| CCR5-768 | + | GAAUCAGCAAUUCUCUG | 17 | 1121 |
| CCR5-769 | + | UCUUUUAAAUAUACAUA | 17 | 1122 |
| CCR5-770 | + | UAUACAUAAGGAACUUU | 17 | 1123 |
| CCR5-771 | + | AGGAACUUUCGGAGUGA | 17 | 1124 |
| CCR5-772 | + | GGAACUUUCGGAGUGAA | 17 | 1125 |
| CCR5-773 | + | UAACUUGAUGCAUGUGA | 17 | 1126 |
| CCR5-774 | + | AACUUGAUGCAUGUGAA | 17 | 1127 |
| CCR5-775 | + | ACUUGAUGCAUGUGAAG | 17 | 1128 |
| CCR5-776 | + | GUGAAGGGGAGAUAAAA | 17 | 1129 |
| CCR5-777 | + | AUCAACAUAUUUUGAUU | 17 | 1130 |
| CCR5-778 | + | UGGCUUUCUAUAAUUGA | 17 | 1131 |
| CCR5-779 | + | GGCUUUCUAUAAUUGAU | 17 | 1132 |
| CCR5-780 | + | AACAGAUGCCAAAUAAA | 17 | 1133 |
| CCR5-781 | + | CACCCCACCCCCAGCCC | 17 | 1134 |
| CCR5-782 | + | AUGUGCACAACUCUGAC | 17 | 1135 |
| CCR5-783 | + | UGUGCACAACUCUGACU | 17 | 1136 |
| CCR5-784 | + | UAUUUCCUGCUCCCCAG | 17 | 1137 |
| CCR5-785 | + | CCUGCUCCCCAGUGGAU | 17 | 1138 |
| CCR5-786 | + | CUGCUCCCCAGUGGAUC | 17 | 1139 |
| CCR5-787 | + | AACUGAGCUUGCUCGCU | 17 | 1140 |
| CCR5-788 | + | ACUGAGCUUGCUCGCUC | 17 | 1141 |
| CCR5-789 | + | GCUCGGGAGCCUCUUGC | 17 | 1142 |
| CCR5-790 | + | GCAUUUGCAGAAGCGUU | 17 | 1143 |
| CCR5-791 | + | GUUUGGCAAUGUGCUUU | 17 | 1144 |
| CCR5-792 | + | UUUGGAAGAAGACUAAG | 17 | 1145 |
| CCR5-793 | + | GAACUUCUCCCCGACAA | 17 | 1146 |
| CCR5-794 | + | GACAAAGGCAUAGAUGA | 17 | 1147 |
| CCR5-795 | + | ACAAAGGCAUAGAUGAU | 17 | 1148 |
| CCR5-796 | + | CAAAGGCAUAGAUGAUG | 17 | 1149 |
| CCR5-797 | + | CUGUCACCUGCAUAGCU | 17 | 1150 |
| CCR5-798 | + | AGCUACUGCAAUUAUUC | 17 | 1151 |
| CCR5-799 | + | UCAGGCCAAAGAAUUCC | 17 | 1152 |
| CCR5-800 | + | GCCAAAGAAUUCCUGGA | 17 | 1153 |
| CCR5-801 | + | AUUCCUGGAAGGUGUUC | 17 | 1154 |
| CCR5-802 | + | GGAAGGUGUUCAGGAGA | 17 | 1155 |
| CCR5-803 | + | GAGAAGGACAAUGUUGU | 17 | 1156 |
| CCR5-804 | + | AGAAGGACAAUGUUGUA | 17 | 1157 |
| CCR5-805 | + | AAAAUAAACAAUCAUGA | 17 | 1158 |
| CCR5-806 | + | ACCGAAGCAGAGUUUUU | 17 | 1159 |
| CCR5-807 | + | AUGACCAUGACAAGCAG | 17 | 1160 |
| CCR5-808 | + | CCAUGACAAGCAGCGGC | 17 | 1161 |
| CCR5-809 | + | UGACUAUCUUUAAUGUC | 17 | 1162 |
| CCR5-810 | + | AAUUGAUACUGACUGUA | 17 | 1163 |
| CCR5-811 | + | UGGAAAAUGAGAGCUGC | 17 | 1164 |

Table 1E provides targeting domains for knocking out the CCR5 gene. In an embodiment, the targeting domain is the exact complement of the target domain. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks.

TABLE 1E

| | | | Target | SEQ |
|---|---|---|---|---|
| | DNA | | Site | ID |
| gRNA Name | Strand | Targeting Domain | Length | NO |
| CCR5-812 | − | AUGACAUCAAUUAUUAUACA | 20 | 1165 |
| CCR5-813 | − | UGACAUCAAUUAUUAUACAU | 20 | 1166 |
| CCR5-814 | − | AGCCCUGCCAAAAAAUCAAU | 20 | 1167 |
| CCR5-815 | − | UGGUGUUCAUCUUUGGUUUU | 20 | 1168 |
| CCR5-816 | − | UCCUGAUAAACUGCAAAAGG | 20 | 1169 |
| CCR5-817 | − | UGAUAAACUGCAAAAGGCUG | 20 | 1170 |
| CCR5-818 | − | UUCCUUCUUACUGUCCCCUU | 20 | 1171 |
| CCR5-819 | − | GCUCACUAUGCUGCCGCCCA | 20 | 1172 |
| CCR5-820 | − | CUCACUAUGCUGCCGCCCAG | 20 | 1173 |
| CCR5-821 | − | UGCUGCCGCCCAGUGGGACU | 20 | 1174 |
| CCR5-822 | − | GCUGCCGCCCAGUGGGACUU | 20 | 1175 |
| CCR5-823 | − | UACAAUGUGUCAACUCUUGA | 20 | 1176 |
| CCR5-824 | − | CUAUUUUAUAGGCUUCUUCU | 20 | 1177 |
| CCR5-825 | − | UAUUUUAUAGGCUUCUUCUC | 20 | 1178 |
| CCR5-826 | − | GCUGUGUUUGCUUUAAAAGC | 20 | 1179 |
| CCR5-827 | − | AAAAGCCAGGACGGUCACCU | 20 | 1180 |
| CCR5-828 | − | AAAGCCAGGACGGUCACCUU | 20 | 1181 |
| CCR5-829 | − | GUGGUGACAAGUGUGAUCAC | 20 | 1182 |
| CCR5-830 | − | GGCUGUGUUUGCGUCUCUCC | 20 | 1183 |
| CCR5-831 | − | GCUGUGUUUGCGUCUCUCCC | 20 | 1184 |
| CCR5-832 | − | ACAUCAAUUAUUAUACA | 17 | 1185 |
| CCR5-833 | − | CAUCAAUUAUUAUACAU | 17 | 1186 |
| CCR5-834 | − | CCUGCCAAAAAAUCAAU | 17 | 1187 |
| CCR5-835 | − | UGUUCAUCUUUGGUUUU | 17 | 1188 |
| CCR5-836 | − | UGAUAAACUGCAAAAGG | 17 | 1189 |
| CCR5-837 | − | UAAACUGCAAAAGGCUG | 17 | 1190 |
| CCR5-838 | − | CUUCUUACUGUCCCCUU | 17 | 1191 |
| CCR5-839 | − | CACUAUGCUGCCGCCCA | 17 | 1192 |
| CCR5-840 | − | ACUAUGCUGCCGCCCAG | 17 | 1193 |
| CCR5-841 | − | UGCCGCCCAGUGGGACU | 17 | 1194 |
| CCR5-842 | − | GCCGCCCAGUGGGACUU | 17 | 1195 |
| CCR5-843 | − | AAUGUGUCAACUCUUGA | 17 | 1196 |
| CCR5-844 | − | UUUUAUAGGCUUCUUCU | 17 | 1197 |
| CCR5-845 | − | UUUAUAGGCUUCUUCUC | 17 | 1198 |
| CCR5-846 | − | GUGUUUGCUUUAAAAGC | 17 | 1199 |
| CCR5-847 | − | AGCCAGGACGGUCACCU | 17 | 1200 |
| CCR5-848 | − | GCCAGGACGGUCACCUU | 17 | 1201 |
| CCR5-849 | − | GUGACAAGUGUGAUCAC | 17 | 1202 |
| CCR5-850 | − | UGUGUUUGCGUCUCUCC | 17 | 1203 |
| CCR5-851 | − | GUGUUUGCGUCUCUCCC | 17 | 1204 |
| CCR5-852 | + | GCUUUUAAAGCAAACACAGC | 20 | 1205 |
| CCR5-853 | + | GCCAGGUACCUAUCGAUUGU | 20 | 1206 |
| CCR5-854 | + | CCAGGUACCUAUCGAUUGUC | 20 | 1207 |
| CCR5-855 | + | AGGUACCUAUCGAUUGUCAG | 20 | 1208 |
| CCR5-856 | + | UAUCGAUUGUCAGGAGGAUG | 20 | 1209 |
| CCR5-857 | + | CGAUUGUCAGGAGGAUGAUG | 20 | 1210 |
| CCR5-858 | + | GAGGAUGAUGAAGAAGAUUC | 20 | 1211 |
| CCR5-859 | + | GGAUGAUGAAGAAGAUUCCA | 20 | 1212 |
| CCR5-860 | + | UGAUGAAGAAGAUUCCAGAG | 20 | 1213 |
| CCR5-861 | + | CAGAGAAGAAGCCUAUAAAA | 20 | 1214 |
| CCR5-862 | + | CUAUAAAAUAGAGCCCUGUC | 20 | 1215 |
| CCR5-863 | + | AUUGUAUUUCCAAAGUCCCA | 20 | 1216 |
| CCR5-864 | + | UCCCACUGGGCGGCAGCAUA | 20 | 1217 |
| CCR5-865 | + | GGGCGGCAGCAUAGUGAGCC | 20 | 1218 |
| CCR5-866 | + | CGGCAGCAUAGUGAGCCCAG | 20 | 1219 |
| CCR5-867 | + | GGCAGCAUAGUGAGCCCAGA | 20 | 1220 |
| CCR5-868 | + | GCAGCAUAGUGAGCCCAGAA | 20 | 1221 |
| CCR5-869 | + | UGAGCCCAGAAGGGGACAGU | 20 | 1222 |
| CCR5-870 | + | GCCCAGAAGGGGACAGUAAG | 20 | 1223 |
| CCR5-871 | + | CCCAGAAGGGGACAGUAAGA | 20 | 1224 |
| CCR5-872 | + | AGUAAGAAGGAAAAACAGGU | 20 | 1225 |
| CCR5-873 | + | ACAGGUCAGAGAUGGCCAGG | 20 | 1226 |
| CCR5-874 | + | UUCAGCCUUUUGCAGUUUAU | 20 | 1227 |
| CCR5-875 | + | GCCUUUUGCAGUUUAUCAGG | 20 | 1228 |
| CCR5-876 | + | CUUUUGCAGUUUAUCAGGAU | 20 | 1229 |
| CCR5-877 | + | UGUUGCCCACAAAACCAAAG | 20 | 1230 |
| CCR5-878 | + | AAAACCAAAGAUGAACACCA | 20 | 1231 |
| CCR5-879 | + | CAAAGAUGAACACCAGUGAG | 20 | 1232 |
| CCR5-880 | + | GAUGAACACCAGUGAGUAGA | 20 | 1233 |
| CCR5-881 | + | AUGAACACCAGUGAGUAGAG | 20 | 1234 |
| CCR5-882 | + | ACCAGUGAGUAGAGCGGAGG | 20 | 1235 |
| CCR5-883 | + | CCAGUGAGUAGAGCGGAGGC | 20 | 1236 |
| CCR5-884 | + | GAGUAGAGCGGAGGCAGGAG | 20 | 1237 |
| CCR5-885 | + | GCUUCACAUUGAUUUUUUGG | 20 | 1238 |
| CCR5-886 | + | AUAAUAAUUGAUGUCAUAGA | 20 | 1239 |
| CCR5-887 | + | UUUAAAGCAAACACAGC | 17 | 1240 |
| CCR5-888 | + | AGGUACCUAUCGAUUGU | 17 | 1241 |
| CCR5-889 | + | GGUACCUAUCGAUUGUC | 17 | 1242 |
| CCR5-890 | + | UACCUAUCGAUUGUCAG | 17 | 1243 |
| CCR5-891 | + | CGAUUGUCAGGAGGAUG | 17 | 1244 |
| CCR5-892 | + | UUGUCAGGAGGAUGAUG | 17 | 1245 |
| CCR5-893 | + | GAUGAUGAAGAAGAUUC | 17 | 1246 |
| CCR5-894 | + | UGAUGAAGAAGAUUCCA | 17 | 1247 |
| CCR5-895 | + | UGAAGAAGAUUCCAGAG | 17 | 1248 |
| CCR5-896 | + | AGAAGAAGCCUAUAAAA | 17 | 1249 |
| CCR5-897 | + | UAAAAUAGAGCCCUGUC | 17 | 1250 |
| CCR5-898 | + | GUAUUUCCAAAGUCCCA | 17 | 1251 |
| CCR5-899 | + | CACUGGGCGGCAGCAUA | 17 | 1252 |
| CCR5-900 | + | CGGCAGCAUAGUGAGCC | 17 | 1253 |
| CCR5-901 | + | CAGCAUAGUGAGCCCAG | 17 | 1254 |
| CCR5-902 | + | AGCAUAGUGAGCCCAGA | 17 | 1255 |
| CCR5-903 | + | GCAUAGUGAGCCCAGAA | 17 | 1256 |
| CCR5-904 | + | GCCCAGAAGGGGACAGU | 17 | 1257 |
| CCR5-905 | + | CAGAAGGGGACAGUAAG | 17 | 1258 |
| CCR5-906 | + | AGAAGGGGACAGUAAGA | 17 | 1259 |
| CCR5-907 | + | AAGAAGGAAAAACAGGU | 17 | 1260 |
| CCR5-908 | + | GGUCAGAGAUGGCCAGG | 17 | 1261 |
| CCR5-909 | + | AGCCUUUUGCAGUUUAU | 17 | 1262 |
| CCR5-910 | + | UUUUGCAGUUUAUCAGG | 17 | 1263 |
| CCR5-911 | + | UUGCAGUUUAUCAGGAU | 17 | 1264 |
| CCR5-912 | + | UGCCCACAAAACCAAAG | 17 | 1265 |
| CCR5-913 | + | ACCAAAGAUGAACACCA | 17 | 1266 |
| CCR5-914 | + | AGAUGAACACCAGUGAG | 17 | 1267 |
| CCR5-915 | + | GAACACCAGUGAGUAGA | 17 | 1268 |
| CCR5-916 | + | AACACCAGUGAGUAGAG | 17 | 1269 |
| CCR5-917 | + | AGUGAGUAGAGCGGAGG | 17 | 1270 |
| CCR5-918 | + | GUGAGUAGAGCGGAGGC | 17 | 1271 |
| CCR5-919 | + | UAGAGCGGAGGCAGGAG | 17 | 1272 |
| CCR5-920 | + | UCACAUUGAUUUUUUGG | 17 | 1273 |
| CCR5-921 | + | AUAAUUGAUGUCAUAGA | 17 | 1274 |
| CCR5-922 | − | CCAUACAGUCAGUAUCAAUU | 20 | 1275 |
| CCR5-923 | − | CAUACAGUCAGUAUCAAUUC | 20 | 1276 |
| CCR5-924 | − | ACAGUCAGUAUCAAUUCUGG | 20 | 1277 |
| CCR5-925 | − | AGACAUUAAAGAUAGUCAUC | 20 | 1278 |
| CCR5-926 | − | GACAUUAAAGAUAGUCAUCU | 20 | 1279 |
| CCR5-927 | − | UUGUCAUGGUCAUCUGCUAC | 20 | 1280 |
| CCR5-928 | − | UGUCAUGGUCAUCUGCUACU | 20 | 1281 |
| CCR5-929 | − | GUCAUGGUCAUCUGCUACUC | 20 | 1282 |
| CCR5-930 | − | CUAAAAACUCUGCUUCGGUG | 20 | 1283 |
| CCR5-931 | − | AACUCUGCUUCGGUGUCGAA | 20 | 1284 |
| CCR5-932 | − | CUCUGCUUCGGUGUCGAAAU | 20 | 1285 |
| CCR5-933 | − | UGCUUCGGUGUCGAAAUGAG | 20 | 1286 |
| CCR5-934 | − | UUCGGUGUCGAAAUGAGAAG | 20 | 1287 |
| CCR5-935 | − | CGAAAUGAGAAGAAGAGGCA | 20 | 1288 |
| CCR5-936 | − | AGAAGAAGAGGCACAGGGCU | 20 | 1289 |
| CCR5-937 | − | AUGAUUGUUUAUUUUCUCUU | 20 | 1290 |
| CCR5-938 | − | CCUACAACAUUGUCCUUCUC | 20 | 1291 |
| CCR5-939 | − | UCCUUCUCCUGAACACCUUC | 20 | 1292 |
| CCR5-940 | − | CCUUCUCCUGAACACCUUCC | 20 | 1293 |
| CCR5-941 | − | CCUUCCAGGAAUUCUUUGGC | 20 | 1294 |
| CCR5-942 | − | AUUGCAGUAGCUCUAACAGG | 20 | 1295 |
| CCR5-943 | − | GGACCAAGCUAUGCAGGUGA | 20 | 1296 |
| CCR5-944 | − | UAUGCAGGUGACAGAGACUC | 20 | 1297 |
| CCR5-945 | − | AUGCAGGUGACAGAGACUCU | 20 | 1298 |
| CCR5-946 | − | CCCCAUCAUCUAUGCCUUUG | 20 | 1299 |
| CCR5-947 | − | CCCAUCAUCUAUGCCUUUGU | 20 | 1300 |
| CCR5-948 | − | CCAUCAUCUAUGCCUUUGUC | 20 | 1301 |
| CCR5-949 | − | CAUCAUCUAUGCCUUUGUCG | 20 | 1302 |
| CCR5-950 | − | UCAUCUAUGCCUUUGUCGGG | 20 | 1303 |
| CCR5-951 | − | GCCUUUGUCGGGGAGAAGUU | 20 | 1304 |
| CCR5-952 | − | AUGCUGUUCUAUUUUCCAGC | 20 | 1305 |
| CCR5-953 | − | UAUUUUCCAGCAAGAGGCUC | 20 | 1306 |
| CCR5-954 | − | UUCCAGCAAGAGGCUCCCGA | 20 | 1307 |
| CCR5-955 | − | CUCAGUUUACACCCGAUCCA | 20 | 1308 |
| CCR5-956 | − | UCAGUUUACACCCGAUCCAC | 20 | 1309 |
| CCR5-957 | − | CAGUUUACACCCGAUCCACU | 20 | 1310 |
| CCR5-958 | − | AGUUUACACCCGAUCCACUG | 20 | 1311 |
| CCR5-959 | − | ACACCCGAUCCACUGGGGAG | 20 | 1312 |
| CCR5-960 | − | CACCCGAUCCACUGGGGAGC | 20 | 1313 |
| CCR5-961 | − | CUGGGGAGCAGGAAAUAUCU | 20 | 1314 |
| CCR5-962 | − | AAUAUCUGUGGGCUUGUGAC | 20 | 1315 |
| CCR5-963 | − | GGCUUGUGACACGGACUCAA | 20 | 1316 |
| CCR5-964 | − | AAGUGGGCUGGUGACCCAGU | 20 | 1317 |
| CCR5-965 | − | GCUUAGUUUUCAUACACAGC | 20 | 1318 |
| CCR5-966 | − | GUUUUCAUACACAGCCUGGG | 20 | 1319 |
| CCR5-967 | − | UUUUCAUACACAGCCUGGGC | 20 | 1320 |
| CCR5-968 | − | UUUCAUACACAGCCUGGGCU | 20 | 1321 |
| CCR5-969 | − | AUACACAGCCUGGGCUGGGG | 20 | 1322 |
| CCR5-970 | − | UACACAGCCUGGGCUGGGGG | 20 | 1323 |
| CCR5-971 | − | CAGCCUGGGCUGGGGGUGGG | 20 | 1324 |
| CCR5-972 | − | AGCCUGGGCUGGGGGUGGGG | 20 | 1325 |
| CCR5-973 | − | GCCUGGGCUGGGGGUGGGGU | 20 | 1326 |
| CCR5-974 | − | CUGGGCUGGGGGUGGGGUGG | 20 | 1327 |
| CCR5-975 | − | GUGGGAGAGGUCUUUUUUAA | 20 | 1328 |
| CCR5-976 | − | UGGGAGAGGUCUUUUUUAAA | 20 | 1329 |
| CCR5-977 | − | UUAAAAGGAAGUUACUGUUA | 20 | 1330 |
| CCR5-978 | − | AAAAGGAAGUUACUGUUAUA | 20 | 1331 |
| CCR5-979 | − | UCUUUUAAGCCCAUCAAUUA | 20 | 1332 |
| CCR5-980 | − | AGCCAAAUCAAAAUAUGUUG | 20 | 1333 |
| CCR5-981 | − | UGACAAACUCUCCCUUCACU | 20 | 1334 |
| CCR5-982 | − | AGUUCCUUAUGUAUAUUUAA | 20 | 1335 |
| CCR5-983 | − | GUAUAUUUAAAAGAAAGCCU | 20 | 1336 |
| CCR5-984 | − | AUAUUUAAAAGAAAGCCUCA | 20 | 1337 |
| CCR5-985 | − | CCUCAGAGAAUUGCUGAUUC | 20 | 1338 |
| CCR5-986 | − | UGAUUCUUGAGUUUAGUGAU | 20 | 1339 |
| CCR5-987 | − | CUUGAGUUUAGUGAUCUGAA | 20 | 1340 |
| CCR5-988 | − | CAGAAAUACCAAAAUUAUUU | 20 | 1341 |
| CCR5-989 | − | AAACAGGUCUUUGUCUUGCU | 20 | 1342 |
| CCR5-990 | − | AACAGGUCUUUGUCUUGCUA | 20 | 1343 |
| CCR5-991 | − | ACAGGUCUUUGUCUUGCUAU | 20 | 1344 |
| CCR5-992 | − | CAGGUCUUUGUCUUGCUAUG | 20 | 1345 |
| CCR5-993 | − | GGUCUUUGUCUUGCUAUGGG | 20 | 1346 |
| CCR5-994 | − | UUGCUAUGGGGAGAAAAGAC | 20 | 1347 |
| CCR5-995 | − | AGACAUGAAUAUGAUUAGUA | 20 | 1348 |
| CCR5-996 | − | GUUAAUAAGUUUCACUGACU | 20 | 1349 |
| CCR5-997 | − | UUUCACUGACUUAGAACCAG | 20 | 1350 |
| CCR5-998 | − | UCACUGACUUAGAACCAGGC | 20 | 1351 |
| CCR5-999 | − | CCAGGCGAGAGACUUGUGGC | 20 | 1352 |
| CCR5-1000 | − | CAGGCGAGAGACUUGUGGCC | 20 | 1353 |
| CCR5-1001 | − | AGGCGAGAGACUUGUGGCCU | 20 | 1354 |
| CCR5-1002 | − | GCGAGAGACUUGUGGCCUGG | 20 | 1355 |
| CCR5-1003 | − | AGACUUGUGGCCUGGGAGAG | 20 | 1356 |
| CCR5-1004 | − | GACUUGUGGCCUGGGAGAGC | 20 | 1357 |
| CCR5-1005 | − | ACUUGUGGCCUGGGAGAGCU | 20 | 1358 |
| CCR5-1006 | − | CUUGUGGCCUGGGAGAGCUG | 20 | 1359 |
| CCR5-1007 | − | GAGCUGGGGAAGCUUCUUAA | 20 | 1360 |
| CCR5-1008 | − | GCUGGGGAAGCUUCUUAAAU | 20 | 1361 |
| CCR5-1009 | − | GGGGAAGCUUCUUAAAUGAG | 20 | 1362 |
| CCR5-1010 | − | GGGAAGCUUCUUAAAUGAGA | 20 | 1363 |
| CCR5-1011 | − | CUUCUUAAAUGAGAAGGAAU | 20 | 1364 |
| CCR5-1012 | − | UAAAUGAGAAGGAAUUUGAG | 20 | 1365 |
| CCR5-1013 | − | UCAUCUAUUGCUGGCAAAGA | 20 | 1366 |
| CCR5-1014 | − | AGCCUCACUGCAAGCACUGC | 20 | 1367 |
| CCR5-1015 | − | UGCAUGGGCAAGCUUGGCUG | 20 | 1368 |
| CCR5-1016 | − | AUGGGCAAGCUUGGCUGUAG | 20 | 1369 |
| CCR5-1017 | − | UGGGCAAGCUUGGCUGUAGA | 20 | 1370 |
| CCR5-1018 | − | AGCUUGGCUGUAGAAGGAGA | 20 | 1371 |
| CCR5-1019 | − | GUAGAAGGAGACAGAGCUGG | 20 | 1372 |
| CCR5-1020 | − | UAGAAGGAGACAGAGCUGGU | 20 | 1373 |
| CCR5-1021 | − | AGAAGGAGACAGAGCUGGUU | 20 | 1374 |
| CCR5-1022 | − | ACAGAGCUGGUUGGGAAGAC | 20 | 1375 |
| CCR5-1023 | − | CAGAGCUGGUUGGGAAGACA | 20 | 1376 |
| CCR5-1024 | − | AGAGCUGGUUGGGAAGACAU | 20 | 1377 |
| CCR5-1025 | − | GAGCUGGUUGGGAAGACAUG | 20 | 1378 |
| CCR5-1026 | − | GCUGGUUGGGAAGACAUGGG | 20 | 1379 |
| CCR5-1027 | − | CUGGUUGGGAAGACAUGGGG | 20 | 1380 |
| CCR5-1028 | − | GUUGGGAAGACAUGGGGAGG | 20 | 1381 |
| CCR5-1029 | − | AGGAAGGACAAGGCUAGAUC | 20 | 1382 |
| CCR5-1030 | − | AAGGACAAGGCUAGAUCAUG | 20 | 1383 |
| CCR5-1031 | − | GGCAUUGCUCCGUCUAAGUC | 20 | 1384 |
| CCR5-1032 | − | UGCUCCGUCUAAGUCAUGAG | 20 | 1385 |
| CCR5-1033 | − | CGUCUAAGUCAUGAGCUGAG | 20 | 1386 |
| CCR5-1034 | − | GUCUAAGUCAUGAGCUGAGC | 20 | 1387 |
| CCR5-1035 | − | UCUAAGUCAUGAGCUGAGCA | 20 | 1388 |
| CCR5-1036 | − | GGAGAUCCUGGUUGGUGUUG | 20 | 1389 |
| CCR5-1037 | − | GAAGGUUUACUCUGUGGCCA | 20 | 1390 |
| CCR5-1038 | − | AAGGUUUACUCUGUGGCCAA | 20 | 1391 |
| CCR5-1039 | − | GGUUUACUCUGUGGCCAAAG | 20 | 1392 |
| CCR5-1040 | − | CUCUGUGGCCAAAGGAGGGU | 20 | 1393 |
| CCR5-1041 | − | UCUGUGGCCAAAGGAGGGUC | 20 | 1394 |
| CCR5-1042 | − | GUGGCCAAAGGAGGGUCAGG | 20 | 1395 |
| CCR5-1043 | − | CCAAAGGAGGGUCAGGAAGG | 20 | 1396 |
| CCR5-1044 | − | GGUCAGGAAGGAUGAGCAUU | 20 | 1397 |
| CCR5-1045 | − | GAAGGAUGAGCAUUUAGGGC | 20 | 1398 |
| CCR5-1046 | − | AAGGAUGAGCAUUUAGGGCA | 20 | 1399 |
| CCR5-1047 | − | ACCACCAACAGCCCUCAGGU | 20 | 1400 |
| CCR5-1048 | − | CCAACAGCCCUCAGGUCAGG | 20 | 1401 |
| CCR5-1049 | − | AACAGCCCUCAGGUCAGGGU | 20 | 1402 |
| CCR5-1050 | − | GCCUCUGCUAAGCUCAAGGC | 20 | 1403 |
| CCR5-1051 | − | CUCUGCUAAGCUCAAGGCGU | 20 | 1404 |
| CCR5-1052 | − | GCUAAGCUCAAGGCGUGAGG | 20 | 1405 |
| CCR5-1053 | − | CUAAGCUCAAGGCGUGAGGA | 20 | 1406 |
| CCR5-1054 | − | UAAGCUCAAGGCGUGAGGAU | 20 | 1407 |
| CCR5-1055 | − | GCUCAAGGCGUGAGGAUGGG | 20 | 1408 |
| CCR5-1056 | − | CUCAAGGCGUGAGGAUGGGA | 20 | 1409 |
| CCR5-1057 | − | CAAGGCGUGAGGAUGGGAAG | 20 | 1410 |
| CCR5-1058 | − | AAGGCGUGAGGAUGGGAAGG | 20 | 1411 |
| CCR5-1059 | − | AGGCGUGAGGAUGGGAAGGA | 20 | 1412 |
| CCR5-1060 | − | GGAAGGAGGGAGGUAUUCGU | 20 | 1413 |
| CCR5-1061 | − | GGAGGGAGGUAUUCGUAAGG | 20 | 1414 |
| CCR5-1062 | − | GAGGGAGGUAUUCGUAAGGA | 20 | 1415 |
| CCR5-1063 | − | AGGGAGGUAUUCGUAAGGAU | 20 | 1416 |
| CCR5-1064 | − | GAGGUAUUCGUAAGGAUGGG | 20 | 1417 |
| CCR5-1065 | − | AGGUAUUCGUAAGGAUGGGA | 20 | 1418 |
| CCR5-1066 | − | GUAUUCGUAAGGAUGGGAAG | 20 | 1419 |
| CCR5-1067 | − | UAUUCGUAAGGAUGGGAAGG | 20 | 1420 |
| CCR5-1068 | − | AUUCGUAAGGAUGGGAAGGA | 20 | 1421 |
| CCR5-1069 | − | GGGAGGUAUUCGUGCAGCAU | 20 | 1422 |
| CCR5-1070 | − | GAGGUAUUCGUGCAGCAUAU | 20 | 1423 |
| CCR5-1071 | − | UCGUGCAGCAUAUGAGGAUG | 20 | 1424 |
| CCR5-1072 | − | AUAUGAGGAUGCAGAGUCAG | 20 | 1425 |
| CCR5-1073 | − | AGGAUGCAGAGUCAGCAGAA | 20 | 1426 |
| CCR5-1074 | − | GGAUGCAGAGUCAGCAGAAC | 20 | 1427 |
| CCR5-1075 | − | GCAGAGUCAGCAGAACUGGG | 20 | 1428 |
| CCR5-1076 | − | UCAGCAGAACUGGGGUGGAU | 20 | 1429 |
| CCR5-1077 | − | AGAACUGGGGUGGAUUUGGG | 20 | 1430 |
| CCR5-1078 | − | GAACUGGGGUGGAUUUGGGU | 20 | 1431 |
| CCR5-1079 | − | GGGGUGGAUUUGGGUUGGAA | 20 | 1432 |
| CCR5-1080 | − | GGUGGAUUUGGGUUGGAAGU | 20 | 1433 |
| CCR5-1081 | − | UUUGGGUUGGAAGUGAGGGU | 20 | 1434 |
| CCR5-1082 | − | UGGGUUGGAAGUGAGGGUCA | 20 | 1435 |
| CCR5-1083 | − | GGUUGGAAGUGAGGGUCAGA | 20 | 1436 |
| CCR5-1084 | − | GUUGGAAGUGAGGGUCAGAG | 20 | 1437 |
| CCR5-1085 | − | AGUGAGGGUCAGAGAGGAGU | 20 | 1438 |
| CCR5-1086 | − | UGAGGGUCAGAGAGGAGUCA | 20 | 1439 |
| CCR5-1087 | − | AGGGUCAGAGAGGAGUCAGA | 20 | 1440 |
| CCR5-1088 | − | AUCCCUAGUCUUCAAGCAGA | 20 | 1441 |
| CCR5-1089 | − | UCCCUAGUCUUCAAGCAGAU | 20 | 1442 |
| CCR5-1090 | − | CCUAGUCUUCAAGCAGAUUG | 20 | 1443 |
| CCR5-1091 | − | CAAGCAGAUUGGAGAAACCC | 20 | 1444 |
| CCR5-1092 | − | CCUUGAAAAGACAUCAAGCA | 20 | 1445 |
| CCR5-1093 | − | UGAAAAGACAUCAAGCACAG | 20 | 1446 |
| CCR5-1094 | − | GAAAAGACAUCAAGCACAGA | 20 | 1447 |
| CCR5-1095 | − | AAAGACAUCAAGCACAGAAG | 20 | 1448 |
| CCR5-1096 | − | AAGACAUCAAGCACAGAAGG | 20 | 1449 |
| CCR5-1097 | − | GACAUCAAGCACAGAAGGAG | 20 | 1450 |
| CCR5-1098 | − | ACAUCAAGCACAGAAGGAGG | 20 | 1451 |
| CCR5-1099 | − | AUCAAGCACAGAAGGAGGAG | 20 | 1452 |
| CCR5-1100 | − | UCAAGCACAGAAGGAGGAGG | 20 | 1453 |
| CCR5-1101 | − | AGGAGGAGGAGGUUUAGGUC | 20 | 1454 |
| CCR5-1102 | − | AGGAGGAGGUUUAGGUCAAG | 20 | 1455 |
| CCR5-1103 | − | AGGUUUAGGUCAAGAAGAAG | 20 | 1456 |
| CCR5-1104 | − | AAGAAGAUGGAUUGGUGUAA | 20 | 1457 |
| CCR5-1105 | − | AGAUGGAUUGGUGUAAAAGG | 20 | 1458 |
| CCR5-1106 | − | AAAAGGAUGGGUCUGGUUUG | 20 | 1459 |
| CCR5-1107 | − | AUGGGUCUGGUUUGCAGAGC | 20 | 1460 |
| CCR5-1108 | − | AGACUCCAGGCUGUCUUUCA | 20 | 1461 |
| CCR5-1109 | − | AGAUUUCCUUCCCAUCCCAG | 20 | 1462 |
| CCR5-1110 | − | UUCCCAUCCCAGCUGAAAUA | 20 | 1463 |
| CCR5-1111 | − | CCCAUCCCAGCUGAAAUACU | 20 | 1464 |
| CCR5-1112 | − | CCAUCCCAGCUGAAAUACUG | 20 | 1465 |
| CCR5-1113 | − | CUGAAAUACUGAGGGGUCUC | 20 | 1466 |
| CCR5-1114 | − | UGAAAUACUGAGGGGUCUCC | 20 | 1467 |
| CCR5-1115 | − | AAAUACUGAGGGGUCUCCAG | 20 | 1468 |
| CCR5-1116 | − | AAUACUGAGGGGUCUCCAGG | 20 | 1469 |
| CCR5-1117 | − | UCCAGGAGGAGACUAGAUUU | 20 | 1470 |
| CCR5-1118 | − | GAGACUAGAUUUAUGAAUAC | 20 | 1471 |
| CCR5-1119 | − | GAUUUAUGAAUACACGAGGU | 20 | 1472 |
| CCR5-1120 | − | AAUACACGAGGUAUGAGGUC | 20 | 1473 |
| CCR5-1121 | − | AUACACGAGGUAUGAGGUCU | 20 | 1474 |
| CCR5-1122 | − | GAACAUACUUCAGCUCACAC | 20 | 1475 |
| CCR5-1123 | − | AGCUCACACAUGAGAUCUAG | 20 | 1476 |
| CCR5-1124 | − | CUCACACAUGAGAUCUAGGU | 20 | 1477 |
| CCR5-1125 | − | GAUUACCUAGUAGUCAUUUC | 20 | 1478 |
| CCR5-1126 | − | AGUAGUCAUUUCAUGGGUUG | 20 | 1479 |
| CCR5-1127 | − | GUAGUCAUUUCAUGGGUUGU | 20 | 1480 |
| CCR5-1128 | − | UAGUCAUUUCAUGGGUUGUU | 20 | 1481 |
| CCR5-1129 | − | GUCAUUUCAUGGGUUGUUGG | 20 | 1482 |
| CCR5-1130 | − | UGGGUUGUUGGGAGGAUUCU | 20 | 1483 |
| CCR5-1131 | − | CAAACUCUUAGUUACUCAUU | 20 | 1484 |
| CCR5-1132 | − | AAACUCUUAGUUACUCAUUC | 20 | 1485 |
| CCR5-1133 | − | UUACUCAUUCAGGGAUAGCA | 20 | 1486 |
| CCR5-1134 | − | GGAUAGCACUGAGCAAAGCA | 20 | 1487 |
| CCR5-1135 | − | ACUGAGCAAAGCAUUGAGCA | 20 | 1488 |
| CCR5-1136 | − | CUGAGCAAAGCAUUGAGCAA | 20 | 1489 |
| CCR5-1137 | − | CAUUGAGCAAAGGGGUCCCA | 20 | 1490 |
| CCR5-1138 | − | AGCAAAGGGGUCCCAUAGAG | 20 | 1491 |
| CCR5-1139 | − | CAAAGGGGUCCCAUAGAGGU | 20 | 1492 |
| CCR5-1140 | − | AAAGGGGUCCCAUAGAGGUG | 20 | 1493 |
| CCR5-1141 | − | AAGGGGUCCCAUAGAGGUGA | 20 | 1494 |
| CCR5-1142 | − | CCCAUAGAGGUGAGGGAAGC | 20 | 1495 |
| CCR5-1143 | − | CAUUUAACCGUCAAUAGGCA | 20 | 1496 |
| CCR5-1144 | − | AUUUAACCGUCAAUAGGCAA | 20 | 1497 |
| CCR5-1145 | − | UUUAACCGUCAAUAGGCAAA | 20 | 1498 |
| CCR5-1146 | − | UUAACCGUCAAUAGGCAAAG | 20 | 1499 |
| CCR5-1147 | − | UAACCGUCAAUAGGCAAAGG | 20 | 1500 |
| CCR5-1148 | − | AACCGUCAAUAGGCAAAGGG | 20 | 1501 |
| CCR5-1149 | − | CGUCAAUAGGCAAAGGGGGG | 20 | 1502 |
| CCR5-1150 | − | GUCAAUAGGCAAAGGGGGGA | 20 | 1503 |
| CCR5-1151 | − | GGGGGAAGGGACAUAUUCAU | 20 | 1504 |
| CCR5-1152 | − | GGGGAAGGGACAUAUUCAUU | 20 | 1505 |
| CCR5-1153 | − | UCAUUUGGAAAUAAGCUGCC | 20 | 1506 |
| CCR5-1154 | − | ACCAGCCUCCGUAUUUCAGA | 20 | 1507 |
| CCR5-1155 | − | GCCUCCGUAUUUCAGACUGA | 20 | 1508 |
| CCR5-1156 | − | CCUCCGUAUUUCAGACUGAA | 20 | 1509 |
| CCR5-1157 | − | CUCCGUAUUUCAGACUGAAU | 20 | 1510 |
| CCR5-1158 | − | GUAUUUCAGACUGAAUGGGG | 20 | 1511 |
| CCR5-1159 | − | UAUUUCAGACUGAAUGGGGG | 20 | 1512 |
| CCR5-1160 | − | AUUUCAGACUGAAUGGGGGU | 20 | 1513 |
| CCR5-1161 | − | UUUCAGACUGAAUGGGGGUG | 20 | 1514 |
| CCR5-1162 | − | UUCAGACUGAAUGGGGGUGG | 20 | 1515 |
| CCR5-1163 | − | UCAGACUGAAUGGGGGUGGG | 20 | 1516 |
| CCR5-1164 | − | GAUGCCUUCUCCAGACAAAC | 20 | 1517 |
| CCR5-1165 | − | UCCAGACAAACCAGAAGCAA | 20 | 1518 |
| CCR5-1166 | − | AAAAUCGUCUCUCCCUCCCU | 20 | 1519 |
| CCR5-1167 | − | CGUCUCUCCCUCCCUUUGAA | 20 | 1520 |
| CCR5-1168 | − | AUGAAUAUACCCCUUAGUGU | 20 | 1521 |
| CCR5-1169 | − | GUUUGGGUAUAUUCAUUUCA | 20 | 1522 |
| CCR5-1170 | − | UUUGGGUAUAUUCAUUUCAA | 20 | 1523 |
| CCR5-1171 | − | UUGGGUAUAUUCAUUUCAAA | 20 | 1524 |
| CCR5-1172 | − | GGGUAUAUUCAUUUCAAAGG | 20 | 1525 |
| CCR5-1173 | − | GUAUAUUCAUUUCAAAGGGA | 20 | 1526 |
| CCR5-1174 | − | AUAUUCAUUUCAAAGGGAGA | 20 | 1527 |
| CCR5-1175 | − | AUUCAUUUCAAAGGGAGAGA | 20 | 1528 |
| CCR5-1176 | − | UCAUAUGAUUGUGCACAUAC | 20 | 1529 |
| CCR5-1177 | − | UGCACAUACUUGAGACUGUU | 20 | 1530 |
| CCR5-1178 | − | UACUUGAGACUGUUUUGAAU | 20 | 1531 |
| CCR5-1179 | − | ACUUGAGACUGUUUUGAAUU | 20 | 1532 |
| CCR5-1180 | − | CUUGAGACUGUUUUGAAUUU | 20 | 1533 |
| CCR5-1181 | − | UUGAGACUGUUUUGAAUUUG | 20 | 1534 |
| CCR5-1182 | − | ACCAUCAUAGUACAGGUAAG | 20 | 1535 |
| CCR5-1183 | − | CAUCAUAGUACAGGUAAGGU | 20 | 1536 |
| CCR5-1184 | − | AUCAUAGUACAGGUAAGGUG | 20 | 1537 |
| CCR5-1185 | − | UCAUAGUACAGGUAAGGUGA | 20 | 1538 |
| CCR5-1186 | − | AGGUGAGGGAAUAGUAAGUG | 20 | 1539 |
| CCR5-1187 | − | GUGAGGGAAUAGUAAGUGGU | 20 | 1540 |
| CCR5-1188 | − | AGUAAGUGGUGAGAACUACU | 20 | 1541 |
| CCR5-1189 | − | GUAAGUGGUGAGAACUACUC | 20 | 1542 |
| CCR5-1190 | − | UAAGUGGUGAGAACUACUCA | 20 | 1543 |
| CCR5-1191 | − | UGGUGAGAACUACUCAGGGA | 20 | 1544 |
| CCR5-1192 | − | UACUCAGGGAAUGAAGGUGU | 20 | 1545 |
| CCR5-1193 | − | AAUGAAGGUGUCAGAAUAAU | 20 | 1546 |
| CCR5-1194 | − | GCUACUGACUUUCUCAGCCU | 20 | 1547 |
| CCR5-1195 | − | GACUUUCUCAGCCUCUGAAU | 20 | 1548 |
| CCR5-1196 | − | UCAGCCUCUGAAUAUGAACG | 20 | 1549 |
| CCR5-1197 | − | GUGAGCAUUGUGGCUGUCAG | 20 | 1550 |
| CCR5-1198 | − | UGAGCAUUGUGGCUGUCAGC | 20 | 1551 |
| CCR5-1199 | − | GUGGCUGUCAGCAGGAAGCA | 20 | 1552 |
| CCR5-1200 | − | GCUGUCAGCAGGAAGCAACG | 20 | 1553 |
| CCR5-1201 | − | CUGUCAGCAGGAAGCAACGA | 20 | 1554 |
| CCR5-1202 | − | UGUCAGCAGGAAGCAACGAA | 20 | 1555 |
| CCR5-1203 | − | UUUCCUUUUGCUCUUAAGUU | 20 | 1556 |
| CCR5-1204 | − | UUCCUUUUGCUCUUAAGUUG | 20 | 1557 |
| CCR5-1205 | − | CCUUUUGCUCUUAAGUUGUG | 20 | 1558 |
| CCR5-1206 | − | UGGAGAGUGCAACAGUAGCA | 20 | 1559 |
| CCR5-1207 | − | GUAGCAUAGGACCCUACCCU | 20 | 1560 |
| CCR5-1208 | − | AUUUGCAUAUUCUUAUGUAU | 20 | 1561 |
| CCR5-1209 | − | AUGUGAAAGUUACAAAUUGC | 20 | 1562 |
| CCR5-1210 | − | GAAAGUUACAAAUUGCUUGA | 20 | 1563 |
| CCR5-1211 | − | UACAGUCAGUAUCAAUU | 17 | 1564 |
| CCR5-1212 | − | ACAGUCAGUAUCAAUUC | 17 | 1565 |
| CCR5-1213 | − | GUCAGUAUCAAUUCUGG | 17 | 1566 |
| CCR5-1214 | − | CAUUAAAGAUAGUCAUC | 17 | 1567 |
| CCR5-1215 | − | AUUAAAGAUAGUCAUCU | 17 | 1568 |
| CCR5-1216 | − | UCAUGGUCAUCUGCUAC | 17 | 1569 |
| CCR5-1217 | − | CAUGGUCAUCUGCUACU | 17 | 1570 |
| CCR5-1218 | − | AUGGUCAUCUGCUACUC | 17 | 1571 |
| CCR5-1219 | − | AAAACUCUGCUUCGGUG | 17 | 1572 |
| CCR5-1220 | − | UCUGCUUCGGUGUCGAA | 17 | 1573 |
| CCR5-1221 | − | UGCUUCGGUGUCGAAAU | 17 | 1574 |
| CCR5-1222 | − | UUCGGUGUCGAAAUGAG | 17 | 1575 |
| CCR5-1223 | − | GGUGUCGAAAUGAGAAG | 17 | 1576 |
| CCR5-1224 | − | AAUGAGAAGAAGAGGCA | 17 | 1577 |
| CCR5-1225 | − | AGAAGAGGCACAGGGCU | 17 | 1578 |
| CCR5-1226 | − | AUUGUUUAUUUUCUCUU | 17 | 1579 |
| CCR5-1227 | − | ACAACAUUGUCCUUCUC | 17 | 1580 |
| CCR5-1228 | − | UUCUCCUGAACACCUUC | 17 | 1581 |
| CCR5-1229 | − | UCUCCUGAACACCUUCC | 17 | 1582 |
| CCR5-1230 | − | UCCAGGAAUUCUUUGGC | 17 | 1583 |
| CCR5-1231 | − | GCAGUAGCUCUAACAGG | 17 | 1584 |
| CCR5-1232 | − | CCAAGCUAUGCAGGUGA | 17 | 1585 |
| CCR5-1233 | − | GCAGGUGACAGAGACUC | 17 | 1586 |
| CCR5-1234 | − | CAGGUGACAGAGACUCU | 17 | 1587 |
| CCR5-1235 | − | CAUCAUCUAUGCCUUUG | 17 | 1588 |
| CCR5-1236 | − | AUCAUCUAUGCCUUUGU | 17 | 1589 |
| CCR5-1237 | − | UCAUCUAUGCCUUUGUC | 17 | 1590 |
| CCR5-1238 | − | CAUCUAUGCCUUUGUCG | 17 | 1591 |
| CCR5-1239 | − | UCUAUGCCUUUGUCGGG | 17 | 1592 |
| CCR5-1240 | − | UUUGUCGGGGAGAAGUU | 17 | 1593 |
| CCR5-1241 | − | CUGUUCUAUUUUCCAGC | 17 | 1594 |
| CCR5-1242 | − | UUUCCAGCAAGAGGCUC | 17 | 1595 |
| CCR5-1243 | − | CAGCAAGAGGCUCCCGA | 17 | 1596 |
| CCR5-1244 | − | AGUUUACACCCGAUCCA | 17 | 1597 |
| CCR5-1245 | − | GUUUACACCCGAUCCAC | 17 | 1598 |
| CCR5-1246 | − | UUUACACCCGAUCCACU | 17 | 1599 |
| CCR5-1247 | − | UUACACCCGAUCCACUG | 17 | 1600 |
| CCR5-1248 | − | CCCGAUCCACUGGGGAG | 17 | 1601 |
| CCR5-1249 | − | CCGAUCCACUGGGGAGC | 17 | 1602 |
| CCR5-1250 | − | GGGAGCAGGAAAUAUCU | 17 | 1603 |
| CCR5-1251 | − | AUCUGUGGGCUUGUGAC | 17 | 1604 |
| CCR5-1252 | − | UUGUGACACGGACUCAA | 17 | 1605 |
| CCR5-1253 | − | UGGGCUGGUGACCCAGU | 17 | 1606 |
| CCR5-1254 | − | UAGUUUUCAUACACAGC | 17 | 1607 |
| CCR5-1255 | − | UUCAUACACAGCCUGGG | 17 | 1608 |
| CCR5-1256 | − | UCAUACACAGCCUGGGC | 17 | 1609 |
| CCR5-1257 | − | CAUACACAGCCUGGGCU | 17 | 1610 |
| CCR5-1258 | − | CACAGCCUGGGCUGGGG | 17 | 1611 |
| CCR5-1259 | − | ACAGCCUGGGCUGGGGG | 17 | 1612 |
| CCR5-1260 | − | CCUGGGCUGGGGGUGGG | 17 | 1613 |
| CCR5-1261 | − | CUGGGCUGGGGGUGGGG | 17 | 1614 |
| CCR5-1262 | − | UGGGCUGGGGGUGGGGU | 17 | 1615 |
| CCR5-1263 | − | GGCUGGGGGUGGGGUGG | 17 | 1616 |
| CCR5-1264 | − | GGAGAGGUCUUUUUUAA | 17 | 1617 |
| CCR5-1265 | − | GAGAGGUCUUUUUUAAA | 17 | 1618 |
| CCR5-1266 | − | AAAGGAAGUUACUGUUA | 17 | 1619 |
| CCR5-1267 | − | AGGAAGUUACUGUUAUA | 17 | 1620 |
| CCR5-1268 | − | UUUAAGCCCAUCAAUUA | 17 | 1621 |
| CCR5-1269 | − | CAAAUCAAAAUAUGUUG | 17 | 1622 |
| CCR5-1270 | − | CAAACUCUCCCUUCACU | 17 | 1623 |
| CCR5-1271 | − | UCCUUAUGUAUAUUUAA | 17 | 1624 |
| CCR5-1272 | − | UAUUUAAAAGAAAGCCU | 17 | 1625 |
| CCR5-1273 | − | UUUAAAAGAAAGCCUCA | 17 | 1626 |
| CCR5-1274 | − | CAGAGAAUUGCUGAUUC | 17 | 1627 |
| CCR5-1275 | − | UUCUUGAGUUUAGUGAU | 17 | 1628 |
| CCR5-1276 | − | GAGUUUAGUGAUCUGAA | 17 | 1629 |
| CCR5-1277 | − | AAAUACCAAAAUUAUUU | 17 | 1630 |
| CCR5-1278 | − | CAGGUCUUUGUCUUGCU | 17 | 1631 |
| CCR5-1279 | − | AGGUCUUUGUCUUGCUA | 17 | 1632 |
| CCR5-1280 | − | GGUCUUUGUCUUGCUAU | 17 | 1633 |
| CCR5-1281 | − | GUCUUUGUCUUGCUAUG | 17 | 1634 |
| CCR5-1282 | − | CUUUGUCUUGCUAUGGG | 17 | 1635 |
| CCR5-1283 | − | CUAUGGGGAGAAAAGAC | 17 | 1636 |
| CCR5-1284 | − | CAUGAAUAUGAUUAGUA | 17 | 1637 |
| CCR5-1285 | − | AAUAAGUUUCACUGACU | 17 | 1638 |
| CCR5-1286 | − | CACUGACUUAGAACCAG | 17 | 1639 |
| CCR5-1287 | − | CUGACUUAGAACCAGGC | 17 | 1640 |
| CCR5-1288 | − | GGCGAGAGACUUGUGGC | 17 | 1641 |
| CCR5-1289 | − | GCGAGAGACUUGUGGCC | 17 | 1642 |
| CCR5-1290 | − | CGAGAGACUUGUGGCCU | 17 | 1643 |
| CCR5-1291 | − | AGAGACUUGUGGCCUGG | 17 | 1644 |
| CCR5-1292 | − | CUUGUGGCCUGGGAGAG | 17 | 1645 |
| CCR5-1293 | − | UUGUGGCCUGGGAGAGC | 17 | 1646 |
| CCR5-1294 | − | UGUGGCCUGGGAGAGCU | 17 | 1647 |
| CCR5-1295 | − | GUGGCCUGGGAGAGCUG | 17 | 1648 |
| CCR5-1296 | − | CUGGGGAAGCUUCUUAA | 17 | 1649 |
| CCR5-1297 | − | GGGGAAGCUUCUUAAAU | 17 | 1650 |
| CCR5-1298 | − | GAAGCUUCUUAAAUGAG | 17 | 1651 |
| CCR5-1299 | − | AAGCUUCUUAAAUGAGA | 17 | 1652 |
| CCR5-1300 | − | CUUAAAUGAGAAGGAAU | 17 | 1653 |
| CCR5-1301 | − | AUGAGAAGGAAUUUGAG | 17 | 1654 |
| CCR5-1302 | − | UCUAUUGCUGGCAAAGA | 17 | 1655 |
| CCR5-1303 | − | CUCACUGCAAGCACUGC | 17 | 1656 |
| CCR5-1304 | − | AUGGGCAAGCUUGGCUG | 17 | 1657 |
| CCR5-1305 | − | GGCAAGCUUGGCUGUAG | 17 | 1658 |
| CCR5-1306 | − | GCAAGCUUGGCUGUAGA | 17 | 1659 |
| CCR5-1307 | − | UUGGCUGUAGAAGGAGA | 17 | 1660 |
| CCR5-1308 | − | GAAGGAGACAGAGCUGG | 17 | 1661 |
| CCR5-1309 | − | AAGGAGACAGAGCUGGU | 17 | 1662 |
| CCR5-1310 | − | AGGAGACAGAGCUGGUU | 17 | 1663 |
| CCR5-1311 | − | GAGCUGGUUGGGAAGAC | 17 | 1664 |
| CCR5-1312 | − | AGCUGGUUGGGAAGACA | 17 | 1665 |
| CCR5-1313 | − | GCUGGUUGGGAAGACAU | 17 | 1666 |
| CCR5-1314 | − | CUGGUUGGGAAGACAUG | 17 | 1667 |
| CCR5-1315 | − | GGUUGGGAAGACAUGGG | 17 | 1668 |
| CCR5-1316 | − | GUUGGGAAGACAUGGGG | 17 | 1669 |
| CCR5-1317 | − | GGGAAGACAUGGGGAGG | 17 | 1670 |
| CCR5-1318 | − | AAGGACAAGGCUAGAUC | 17 | 1671 |
| CCR5-1319 | − | GACAAGGCUAGAUCAUG | 17 | 1672 |
| CCR5-1320 | − | AUUGCUCCGUCUAAGUC | 17 | 1673 |
| CCR5-1321 | − | UCCGUCUAAGUCAUGAG | 17 | 1674 |
| CCR5-1322 | − | CUAAGUCAUGAGCUGAG | 17 | 1675 |
| CCR5-1323 | − | UAAGUCAUGAGCUGAGC | 17 | 1676 |
| CCR5-1324 | − | AAGUCAUGAGCUGAGCA | 17 | 1677 |
| CCR5-1325 | − | GAUCCUGGUUGGUGUUG | 17 | 1678 |
| CCR5-1326 | − | GGUUUACUCUGUGGCCA | 17 | 1679 |
| CCR5-1327 | − | GUUUACUCUGUGGCCAA | 17 | 1680 |
| CCR5-1328 | − | UUACUCUGUGGCCAAAG | 17 | 1681 |
| CCR5-1329 | − | UGUGGCCAAAGGAGGGU | 17 | 1682 |
| CCR5-1330 | − | GUGGCCAAAGGAGGGUC | 17 | 1683 |
| CCR5-1331 | − | GCCAAAGGAGGGUCAGG | 17 | 1684 |
| CCR5-1332 | − | AAGGAGGGUCAGGAAGG | 17 | 1685 |
| CCR5-1333 | − | CAGGAAGGAUGAGCAUU | 17 | 1686 |
| CCR5-1334 | − | GGAUGAGCAUUUAGGGC | 17 | 1687 |
| CCR5-1335 | − | GAUGAGCAUUUAGGGCA | 17 | 1688 |
| CCR5-1336 | − | ACCAACAGCCCUCAGGU | 17 | 1689 |
| CCR5-1337 | − | ACAGCCCUCAGGUCAGG | 17 | 1690 |
| CCR5-1338 | − | AGCCCUCAGGUCAGGGU | 17 | 1691 |
| CCR5-1339 | − | UCUGCUAAGCUCAAGGC | 17 | 1692 |
| CCR5-1340 | − | UGCUAAGCUCAAGGCGU | 17 | 1693 |
| CCR5-1341 | − | AAGCUCAAGGCGUGAGG | 17 | 1694 |
| CCR5-1342 | − | AGCUCAAGGCGUGAGGA | 17 | 1695 |
| CCR5-1343 | − | GCUCAAGGCGUGAGGAU | 17 | 1696 |
| CCR5-1344 | − | CAAGGCGUGAGGAUGGG | 17 | 1697 |
| CCR5-1345 | − | AAGGCGUGAGGAUGGGA | 17 | 1698 |
| CCR5-1346 | − | GGCGUGAGGAUGGGAAG | 17 | 1699 |
| CCR5-1347 | − | GCGUGAGGAUGGGAAGG | 17 | 1700 |
| CCR5-1348 | − | CGUGAGGAUGGGAAGGA | 17 | 1701 |
| CCR5-1349 | − | AGGAGGGAGGUAUUCGU | 17 | 1702 |
| CCR5-1350 | − | GGGAGGUAUUCGUAAGG | 17 | 1703 |
| CCR5-1351 | − | GGAGGUAUUCGUAAGGA | 17 | 1704 |
| CCR5-1352 | − | GAGGUAUUCGUAAGGAU | 17 | 1705 |
| CCR5-1353 | − | GUAUUCGUAAGGAUGGG | 17 | 1706 |
| CCR5-1354 | − | UAUUCGUAAGGAUGGGA | 17 | 1707 |
| CCR5-1355 | − | UUCGUAAGGAUGGGAAG | 17 | 1708 |
| CCR5-1356 | − | UCGUAAGGAUGGGAAGG | 17 | 1709 |
| CCR5-1357 | − | CGUAAGGAUGGGAAGGA | 17 | 1710 |
| CCR5-1358 | − | AGGUAUUCGUGCAGCAU | 17 | 1711 |
| CCR5-1359 | − | GUAUUCGUGCAGCAUAU | 17 | 1712 |
| CCR5-1360 | − | UGCAGCAUAUGAGGAUG | 17 | 1713 |
| CCR5-1361 | − | UGAGGAUGCAGAGUCAG | 17 | 1714 |
| CCR5-1362 | − | AUGCAGAGUCAGCAGAA | 17 | 1715 |
| CCR5-1363 | − | UGCAGAGUCAGCAGAAC | 17 | 1716 |
| CCR5-1364 | − | GAGUCAGCAGAACUGGG | 17 | 1717 |
| CCR5-1365 | − | GCAGAACUGGGGUGGAU | 17 | 1718 |
| CCR5-1366 | − | ACUGGGGUGGAUUUGGG | 17 | 1719 |
| CCR5-1367 | − | CUGGGGUGGAUUUGGGU | 17 | 1720 |
| CCR5-1368 | − | GUGGAUUUGGGUUGGAA | 17 | 1721 |
| CCR5-1369 | − | GGAUUUGGGUUGGAAGU | 17 | 1722 |
| CCR5-1370 | − | GGGUUGGAAGUGAGGGU | 17 | 1723 |
| CCR5-1371 | − | GUUGGAAGUGAGGGUCA | 17 | 1724 |
| CCR5-1372 | − | UGGAAGUGAGGGUCAGA | 17 | 1725 |
| CCR5-1373 | − | GGAAGUGAGGGUCAGAG | 17 | 1726 |
| CCR5-1374 | − | GAGGGUCAGAGAGGAGU | 17 | 1727 |
| CCR5-1375 | − | GGGUCAGAGAGGAGUCA | 17 | 1728 |
| CCR5-1376 | − | GUCAGAGAGGAGUCAGA | 17 | 1729 |
| CCR5-1377 | − | CCUAGUCUUCAAGCAGA | 17 | 1730 |
| CCR5-1378 | − | CUAGUCUUCAAGCAGAU | 17 | 1731 |
| CCR5-1379 | − | AGUCUUCAAGCAGAUUG | 17 | 1732 |
| CCR5-1380 | − | GCAGAUUGGAGAAACCC | 17 | 1733 |
| CCR5-1381 | − | UGAAAAGACAUCAAGCA | 17 | 1734 |
| CCR5-1382 | − | AAAGACAUCAAGCACAG | 17 | 1735 |
| CCR5-1383 | − | AAGACAUCAAGCACAGA | 17 | 1736 |
| CCR5-1384 | − | GACAUCAAGCACAGAAG | 17 | 1737 |
| CCR5-1385 | − | ACAUCAAGCACAGAAGG | 17 | 1738 |
| CCR5-1386 | − | AUCAAGCACAGAAGGAG | 17 | 1739 |
| CCR5-1387 | − | UCAAGCACAGAAGGAGG | 17 | 1740 |
| CCR5-1388 | − | AAGCACAGAAGGAGGAG | 17 | 1741 |
| CCR5-1389 | − | AGCACAGAAGGAGGAGG | 17 | 1742 |
| CCR5-1390 | − | AGGAGGAGGUUUAGGUC | 17 | 1743 |
| CCR5-1391 | − | AGGAGGUUUAGGUCAAG | 17 | 1744 |
| CCR5-1392 | − | UUUAGGUCAAGAAGAAG | 17 | 1745 |
| CCR5-1393 | − | AAGAUGGAUUGGUGUAA | 17 | 1746 |
| CCR5-1394 | − | UGGAUUGGUGUAAAAGG | 17 | 1747 |
| CCR5-1395 | − | AGGAUGGGUCUGGUUUG | 17 | 1748 |
| CCR5-1396 | − | GGUCUGGUUUGCAGAGC | 17 | 1749 |
| CCR5-1397 | − | CUCCAGGCUGUCUUUCA | 17 | 1750 |
| CCR5-1398 | − | UUUCCUUCCCAUCCCAG | 17 | 1751 |
| CCR5-1399 | − | CCAUCCCAGCUGAAAUA | 17 | 1752 |
| CCR5-1400 | − | AUCCCAGCUGAAAUACU | 17 | 1753 |
| CCR5-1401 | − | UCCCAGCUGAAAUACUG | 17 | 1754 |
| CCR5-1402 | − | AAAUACUGAGGGGUCUC | 17 | 1755 |
| CCR5-1403 | − | AAUACUGAGGGGUCUCC | 17 | 1756 |
| CCR5-1404 | − | UACUGAGGGGUCUCCAG | 17 | 1757 |
| CCR5-1405 | − | ACUGAGGGGUCUCCAGG | 17 | 1758 |
| CCR5-1406 | − | AGGAGGAGACUAGAUUU | 17 | 1759 |
| CCR5-1407 | − | ACUAGAUUUAUGAAUAC | 17 | 1760 |
| CCR5-1408 | − | UUAUGAAUACACGAGGU | 17 | 1761 |
| CCR5-1409 | − | ACACGAGGUAUGAGGUC | 17 | 1762 |
| CCR5-1410 | − | CACGAGGUAUGAGGUCU | 17 | 1763 |
| CCR5-1411 | − | CAUACUUCAGCUCACAC | 17 | 1764 |
| CCR5-1412 | − | UCACACAUGAGAUCUAG | 17 | 1765 |
| CCR5-1413 | − | ACACAUGAGAUCUAGGU | 17 | 1766 |
| CCR5-1414 | − | UACCUAGUAGUCAUUUC | 17 | 1767 |
| CCR5-1415 | − | AGUCAUUUCAUGGGUUG | 17 | 1768 |
| CCR5-1416 | − | GUCAUUUCAUGGGUUGU | 17 | 1769 |
| CCR5-1417 | − | UCAUUUCAUGGGUUGUU | 17 | 1770 |
| CCR5-1418 | − | AUUUCAUGGGUUGUUGG | 17 | 1771 |
| CCR5-1419 | − | GUUGUUGGGAGGAUUCU | 17 | 1772 |
| CCR5-1420 | − | ACUCUUAGUUACUCAUU | 17 | 1773 |
| CCR5-1421 | − | CUCUUAGUUACUCAUUC | 17 | 1774 |
| CCR5-1422 | − | CUCAUUCAGGGAUAGCA | 17 | 1775 |
| CCR5-1423 | − | UAGCACUGAGCAAAGCA | 17 | 1776 |
| CCR5-1424 | − | GAGCAAAGCAUUGAGCA | 17 | 1777 |
| CCR5-1425 | − | AGCAAAGCAUUGAGCAA | 17 | 1778 |
| CCR5-1426 | − | UGAGCAAAGGGGUCCCA | 17 | 1779 |
| CCR5-1427 | − | AAAGGGGUCCCAUAGAG | 17 | 1780 |
| CCR5-1428 | − | AGGGGUCCCAUAGAGGU | 17 | 1781 |
| CCR5-1429 | − | GGGGUCCCAUAGAGGUG | 17 | 1782 |
| CCR5-1430 | − | GGGUCCCAUAGAGGUGA | 17 | 1783 |
| CCR5-1431 | − | AUAGAGGUGAGGGAAGC | 17 | 1784 |
| CCR5-1432 | − | UUAACCGUCAAUAGGCA | 17 | 1785 |
| CCR5-1433 | − | UAACCGUCAAUAGGCAA | 17 | 1786 |
| CCR5-1434 | − | AACCGUCAAUAGGCAAA | 17 | 1787 |
| CCR5-1435 | − | ACCGUCAAUAGGCAAAG | 17 | 1788 |
| CCR5-1436 | − | CCGUCAAUAGGCAAAGG | 17 | 1789 |
| CCR5-1437 | − | CGUCAAUAGGCAAAGGG | 17 | 1790 |
| CCR5-1438 | − | CAAUAGGCAAAGGGGGG | 17 | 1791 |
| CCR5-1439 | − | AAUAGGCAAAGGGGGGA | 17 | 1792 |
| CCR5-1440 | − | GGAAGGGACAUAUUCAU | 17 | 1793 |
| CCR5-1441 | − | GAAGGGACAUAUUCAUU | 17 | 1794 |
| CCR5-1442 | − | UUUGGAAAUAAGCUGCC | 17 | 1795 |
| CCR5-1443 | − | AGCCUCCGUAUUUCAGA | 17 | 1796 |
| CCR5-1444 | − | UCCGUAUUUCAGACUGA | 17 | 1797 |
| CCR5-1445 | − | CCGUAUUUCAGACUGAA | 17 | 1798 |
| CCR5-1446 | − | CGUAUUUCAGACUGAAU | 17 | 1799 |
| CCR5-1447 | − | UUUCAGACUGAAUGGGG | 17 | 1800 |
| CCR5-1448 | − | UUCAGACUGAAUGGGGG | 17 | 1801 |
| CCR5-1449 | − | UCAGACUGAAUGGGGGU | 17 | 1802 |
| CCR5-1450 | − | CAGACUGAAUGGGGGUG | 17 | 1803 |
| CCR5-1451 | − | AGACUGAAUGGGGGUGG | 17 | 1804 |
| CCR5-1452 | − | GACUGAAUGGGGGUGGG | 17 | 1805 |
| CCR5-1453 | − | GCCUUCUCCAGACAAAC | 17 | 1806 |
| CCR5-1454 | − | AGACAAACCAGAAGCAA | 17 | 1807 |
| CCR5-1455 | − | AUCGUCUCUCCCUCCCU | 17 | 1808 |
| CCR5-1456 | − | CUCUCCCUCCCUUUGAA | 17 | 1809 |
| CCR5-1457 | − | AAUAUACCCCUUAGUGU | 17 | 1810 |
| CCR5-1458 | − | UGGGUAUAUUCAUUUCA | 17 | 1811 |
| CCR5-1459 | − | GGGUAUAUUCAUUUCAA | 17 | 1812 |
| CCR5-1460 | − | GGUAUAUUCAUUUCAAA | 17 | 1813 |
| CCR5-1461 | − | UAUAUUCAUUUCAAAGG | 17 | 1814 |
| CCR5-1462 | − | UAUUCAUUUCAAAGGGA | 17 | 1815 |
| CCR5-1463 | − | UUCAUUUCAAAGGGAGA | 17 | 1816 |
| CCR5-1464 | − | CAUUUCAAAGGGAGAGA | 17 | 1817 |
| CCR5-1465 | − | UAUGAUUGUGCACAUAC | 17 | 1818 |
| CCR5-1466 | − | ACAUACUUGAGACUGUU | 17 | 1819 |
| CCR5-1467 | − | UUGAGACUGUUUUGAAU | 17 | 1820 |
| CCR5-1468 | − | UGAGACUGUUUUGAAUU | 17 | 1821 |
| CCR5-1469 | − | GAGACUGUUUUGAAUUU | 17 | 1822 |
| CCR5-1470 | − | AGACUGUUUUGAAUUUG | 17 | 1823 |
| CCR5-1471 | − | AUCAUAGUACAGGUAAG | 17 | 1824 |
| CCR5-1472 | − | CAUAGUACAGGUAAGGU | 17 | 1825 |
| CCR5-1473 | − | AUAGUACAGGUAAGGUG | 17 | 1826 |
| CCR5-1474 | − | UAGUACAGGUAAGGUGA | 17 | 1827 |
| CCR5-1475 | − | UGAGGGAAUAGUAAGUG | 17 | 1828 |
| CCR5-1476 | − | AGGGAAUAGUAAGUGGU | 17 | 1829 |
| CCR5-1477 | − | AAGUGGUGAGAACUACU | 17 | 1830 |
| CCR5-1478 | − | AGUGGUGAGAACUACUC | 17 | 1831 |
| CCR5-1479 | − | GUGGUGAGAACUACUCA | 17 | 1832 |
| CCR5-1480 | − | UGAGAACUACUCAGGGA | 17 | 1833 |
| CCR5-1481 | − | UCAGGGAAUGAAGGUGU | 17 | 1834 |
| CCR5-1482 | − | GAAGGUGUCAGAAUAAU | 17 | 1835 |
| CCR5-1483 | − | ACUGACUUUCUCAGCCU | 17 | 1836 |
| CCR5-1484 | − | UUUCUCAGCCUCUGAAU | 17 | 1837 |
| CCR5-1485 | − | GCCUCUGAAUAUGAACG | 17 | 1838 |
| CCR5-1486 | − | AGCAUUGUGGCUGUCAG | 17 | 1839 |
| CCR5-1487 | − | GCAUUGUGGCUGUCAGC | 17 | 1840 |
| CCR5-1488 | − | GCUGUCAGCAGGAAGCA | 17 | 1841 |
| CCR5-1489 | − | GUCAGCAGGAAGCAACG | 17 | 1842 |
| CCR5-1490 | − | UCAGCAGGAAGCAACGA | 17 | 1843 |
| CCR5-1491 | − | CAGCAGGAAGCAACGAA | 17 | 1844 |
| CCR5-1492 | − | CCUUUUGCUCUUAAGUU | 17 | 1845 |
| CCR5-1493 | − | CUUUUGCUCUUAAGUUG | 17 | 1846 |
| CCR5-1494 | − | UUUGCUCUUAAGUUGUG | 17 | 1847 |
| CCR5-1495 | − | AGAGUGCAACAGUAGCA | 17 | 1848 |
| CCR5-1496 | − | GCAUAGGACCCUACCCU | 17 | 1849 |
| CCR5-1497 | − | UGCAUAUUCUUAUGUAU | 17 | 1850 |
| CCR5-1498 | − | UGAAAGUUACAAAUUGC | 17 | 1851 |
| CCR5-1499 | − | AGUUACAAAUUGCUUGA | 17 | 1852 |
| CCR5-1500 | + | UUUGUAACUUUCACAUACAU | 20 | 1853 |
| CCR5-1501 | + | AUAUGCAAAUACUAAGAUGU | 20 | 1854 |
| CCR5-1502 | + | AGAAUGUCUUUGACUUGGCC | 20 | 1855 |
| CCR5-1503 | + | AAUGUCUUUGACUUGGCCCA | 20 | 1856 |
| CCR5-1504 | + | CUUUGACUUGGCCCAGAGGG | 20 | 1857 |
| CCR5-1505 | + | UGUUGCACUCUCCACAACUU | 20 | 1858 |
| CCR5-1506 | + | UCUCCACAACUUAAGAGCAA | 20 | 1859 |
| CCR5-1507 | + | CUCCACAACUUAAGAGCAAA | 20 | 1860 |
| CCR5-1508 | + | CAAUGCUCACCGUUCAUAUU | 20 | 1861 |
| CCR5-1509 | + | UCACCGUUCAUAUUCAGAGG | 20 | 1862 |
| CCR5-1510 | + | ACCGUUCAUAUUCAGAGGCU | 20 | 1863 |
| CCR5-1511 | + | UAUUCUGACACCUUCAUUCC | 20 | 1864 |
| CCR5-1512 | + | UCAAGUAUGUGCACAAUCAU | 20 | 1865 |
| CCR5-1513 | + | AUGUGCACAAUCAUAUGAGA | 20 | 1866 |
| CCR5-1514 | + | CACAAUCAUAUGAGACAGAA | 20 | 1867 |
| CCR5-1515 | + | AAAAACCUCUCUCUCUCCCU | 20 | 1868 |
| CCR5-1516 | + | CCUCUCUCUCUCCCUUUGAA | 20 | 1869 |
| CCR5-1517 | + | AAUGAAUAUACCCAAACACU | 20 | 1870 |
| CCR5-1518 | + | AUGAAUAUACCCAAACACUA | 20 | 1871 |
| CCR5-1519 | + | UAAGGGGUAUAUUCAUUUCA | 20 | 1872 |
| CCR5-1520 | + | AAGGGGUAUAUUCAUUUCAA | 20 | 1873 |
| CCR5-1521 | + | AGGGGUAUAUUCAUUUCAAA | 20 | 1874 |
| CCR5-1522 | + | GGGUAUAUUCAUUUCAAAGG | 20 | 1875 |
| CCR5-1523 | + | GGUAUAUUCAUUUCAAAGGG | 20 | 1876 |
| CCR5-1524 | + | GUAUAUUCAUUUCAAAGGGA | 20 | 1877 |
| CCR5-1525 | + | AUAUUCAUUUCAAAGGGAGG | 20 | 1878 |
| CCR5-1526 | + | UUCUGUUGCUUCUGGUUUGU | 20 | 1879 |
| CCR5-1527 | + | UCUGUUGCUUCUGGUUUGUC | 20 | 1880 |
| CCR5-1528 | + | UGUUGCUUCUGGUUUGUCUG | 20 | 1881 |
| CCR5-1529 | + | GGUUUGUCUGGAGAAGGCAU | 20 | 1882 |
| CCR5-1530 | + | GUUUGUCUGGAGAAGGCAUC | 20 | 1883 |
| CCR5-1531 | + | CCCCCCCACCCCCAUUCAGU | 20 | 1884 |
| CCR5-1532 | + | ACCCCCAUUCAGUCUGAAAU | 20 | 1885 |
| CCR5-1533 | + | CCCCCAUUCAGUCUGAAAUA | 20 | 1886 |
| CCR5-1534 | + | GGCUGGUAAAUUGUACUUUU | 20 | 1887 |
| CCR5-1535 | + | UCAAGGCAGCUUAUUUCCAA | 20 | 1888 |
| CCR5-1536 | + | UGCCUAUUGACGGUUAAAUG | 20 | 1889 |
| CCR5-1537 | + | GAUACCUACACUUGUGUGCA | 20 | 1890 |
| CCR5-1538 | + | UUCAGGCUUCCCUCACCUCU | 20 | 1891 |
| CCR5-1539 | + | UCAGGCUUCCCUCACCUCUA | 20 | 1892 |
| CCR5-1540 | + | UGCUUUGCUCAGUGCUAUCC | 20 | 1893 |
| CCR5-1541 | + | UUGCUCAGUGCUAUCCCUGA | 20 | 1894 |
| CCR5-1542 | + | CUAUCCCUGAAUGAGUAACU | 20 | 1895 |
| CCR5-1543 | + | AACUAAGAGUUUGAUGCUUA | 20 | 1896 |
| CCR5-1544 | + | UGCUGCCUGUGGUUGCCUCA | 20 | 1897 |
| CCR5-1545 | + | UAGAAUCCUCCCAACAACCC | 20 | 1898 |
| CCR5-1546 | + | UCCUCACCUAGAUCUCAUGU | 20 | 1899 |
| CCR5-1547 | + | ACCUAGAUCUCAUGUGUGAG | 20 | 1900 |
| CCR5-1548 | + | UUCAUAAAUCUAGUCUCCUC | 20 | 1901 |
| CCR5-1549 | + | UCAUAAAUCUAGUCUCCUCC | 20 | 1902 |
| CCR5-1550 | + | GAGACCCCUCAGUAUUUCAG | 20 | 1903 |
| CCR5-1551 | + | AGACCCCUCAGUAUUUCAGC | 20 | 1904 |
| CCR5-1552 | + | CCCUCAGUAUUUCAGCUGGG | 20 | 1905 |
| CCR5-1553 | + | CCUCAGUAUUUCAGCUGGGA | 20 | 1906 |
| CCR5-1554 | + | CUCAGUAUUUCAGCUGGGAU | 20 | 1907 |
| CCR5-1555 | + | AGUAUUUCAGCUGGGAUGGG | 20 | 1908 |
| CCR5-1556 | + | GUAUUUCAGCUGGGAUGGGA | 20 | 1909 |
| CCR5-1557 | + | CUGGGAUGGGAAGGAAAUCU | 20 | 1910 |
| CCR5-1558 | + | GGGAAGGAAAUCUAUGAAGU | 20 | 1911 |
| CCR5-1559 | + | UAUGAAGUCAGAAGCAUUCA | 20 | 1912 |
| CCR5-1560 | + | AGCAUUCAGUGAAAGACAGC | 20 | 1913 |
| CCR5-1561 | + | GCAUUCAGUGAAAGACAGCC | 20 | 1914 |
| CCR5-1562 | + | AGUGAAAGACAGCCUGGAGU | 20 | 1915 |
| CCR5-1563 | + | AAAGACAGCCUGGAGUCUGG | 20 | 1916 |
| CCR5-1564 | + | UCUGUGCUUGAUGUCUUUUC | 20 | 1917 |
| CCR5-1565 | + | CAAGGGUUUCUCCAAUCUGC | 20 | 1918 |
| CCR5-1566 | + | UCUCCAAUCUGCUUGAAGAC | 20 | 1919 |
| CCR5-1567 | + | CUCCAAUCUGCUUGAAGACU | 20 | 1920 |
| CCR5-1568 | + | UCUGCAUCCUCAUAUGCUGC | 20 | 1921 |
| CCR5-1569 | + | CCUCCCUCCUUCCCAUCCUU | 20 | 1922 |
| CCR5-1570 | + | CUCCUUCCCAUCCUCACGCC | 20 | 1923 |
| CCR5-1571 | + | UCCUCACGCCUUGAGCUUAG | 20 | 1924 |
| CCR5-1572 | + | GAGGCCAUCCUCACCCUGAC | 20 | 1925 |
| CCR5-1573 | + | GGCCAUCCUCACCCUGACCU | 20 | 1926 |
| CCR5-1574 | + | UCCUGACCCUCCUUUGGCCA | 20 | 1927 |
| CCR5-1575 | + | AAACCUUCUGCAACACCAAC | 20 | 1928 |
| CCR5-1576 | + | CUGCUCAGCUCAUGACUUAG | 20 | 1929 |
| CCR5-1577 | + | UGCUCAGCUCAUGACUUAGA | 20 | 1930 |
| CCR5-1578 | + | UUGCCCAUGCAGUGCUUGCA | 20 | 1931 |
| CCR5-1579 | + | ACUCAAAUUCCUUCUCAUUU | 20 | 1932 |
| CCR5-1580 | + | UCUCGCCUGGUUCUAAGUCA | 20 | 1933 |
| CCR5-1581 | + | UGAAACUUAUUAACCAUACC | 20 | 1934 |
| CCR5-1582 | + | GAAACUUAUUAACCAUACCU | 20 | 1935 |
| CCR5-1583 | + | AACUUAUUAACCAUACCUUG | 20 | 1936 |
| CCR5-1584 | + | ACUUAUUAACCAUACCUUGG | 20 | 1937 |
| CCR5-1585 | + | CUUAUUAACCAUACCUUGGA | 20 | 1938 |
| CCR5-1586 | + | UUAUUAACCAUACCUUGGAG | 20 | 1939 |
| CCR5-1587 | + | CCUUGGAGGGGAAAUCACAC | 20 | 1940 |
| CCR5-1588 | + | AGGUAAAAAGUUGUACAUUU | 20 | 1941 |
| CCR5-1589 | + | CUGUUCAGAUCACUAAACUC | 20 | 1942 |
| CCR5-1590 | + | ACUCAAGAAUCAGCAAUUCU | 20 | 1943 |
| CCR5-1591 | + | GCUUUCUUUUAAAUAUACAU | 20 | 1944 |
| CCR5-1592 | + | CUUUCUUUUAAAUAUACAUA | 20 | 1945 |
| CCR5-1593 | + | UAAAUAUACAUAAGGAACUU | 20 | 1946 |
| CCR5-1594 | + | AAAUAUACAUAAGGAACUUU | 20 | 1947 |
| CCR5-1595 | + | AUACAUAAGGAACUUUCGGA | 20 | 1948 |
| CCR5-1596 | + | CAUAAGGAACUUUCGGAGUG | 20 | 1949 |
| CCR5-1597 | + | AUAAGGAACUUUCGGAGUGA | 20 | 1950 |
| CCR5-1598 | + | UAAGGAACUUUCGGAGUGAA | 20 | 1951 |
| CCR5-1599 | + | AGGAACUUUCGGAGUGAAGG | 20 | 1952 |
| CCR5-1600 | + | UUGUCAAUAACUUGAUGCAU | 20 | 1953 |
| CCR5-1601 | + | UCAAUAACUUGAUGCAUGUG | 20 | 1954 |
| CCR5-1602 | + | CAAUAACUUGAUGCAUGUGA | 20 | 1955 |
| CCR5-1603 | + | AAUAACUUGAUGCAUGUGAA | 20 | 1956 |
| CCR5-1604 | + | AUAACUUGAUGCAUGUGAAG | 20 | 1957 |
| CCR5-1605 | + | GAUUUGGCUUUCUAUAAUUG | 20 | 1958 |
| CCR5-1606 | + | UUUAAACAGAUGCCAAAUAA | 20 | 1959 |
| CCR5-1607 | + | AACAGAUGCCAAAUAAAUGG | 20 | 1960 |
| CCR5-1608 | + | ACCCCCAGCCCAGGCUGUGU | 20 | 1961 |
| CCR5-1609 | + | AGCCAUGUGCACAACUCUGA | 20 | 1962 |
| CCR5-1610 | + | UGACUGGGUCACCAGCCCAC | 20 | 1963 |
| CCR5-1611 | + | CAGAUAUUUCCUGCUCCCCA | 20 | 1964 |
| CCR5-1612 | + | AUUUCCUGCUCCCCAGUGGA | 20 | 1965 |
| CCR5-1613 | + | CCCAGUGGAUCGGGUGUAAA | 20 | 1966 |
| CCR5-1614 | + | UGUAAACUGAGCUUGCUCGC | 20 | 1967 |
| CCR5-1615 | + | GUAAACUGAGCUUGCUCGCU | 20 | 1968 |
| CCR5-1616 | + | UAAACUGAGCUUGCUCGCUC | 20 | 1969 |
| CCR5-1617 | + | GCUCGCUCGGGAGCCUCUUG | 20 | 1970 |
| CCR5-1618 | + | CUCGCUCGGGAGCCUCUUGC | 20 | 1971 |
| CCR5-1619 | + | GGGAGCCUCUUGCUGGAAAA | 20 | 1972 |
| CCR5-1620 | + | GGAAAAUAGAACAGCAUUUG | 20 | 1973 |
| CCR5-1621 | + | AAGCGUUUGGCAAUGUGCUU | 20 | 1974 |
| CCR5-1622 | + | AGCGUUUGGCAAUGUGCUUU | 20 | 1975 |
| CCR5-1623 | + | GUUUGGCAAUGUGCUUUUGG | 20 | 1976 |
| CCR5-1624 | + | UGUGCUUUUGGAAGAAGACU | 20 | 1977 |
| CCR5-1625 | + | AGAAGACUAAGAGGUAGUUU | 20 | 1978 |
| CCR5-1626 | + | CCCCGACAAAGGCAUAGAUG | 20 | 1979 |
| CCR5-1627 | + | CCCGACAAAGGCAUAGAUGA | 20 | 1980 |
| CCR5-1628 | + | AUGCAGCAGUGCGUCAUCCC | 20 | 1981 |
| CCR5-1629 | + | CAUAGCUUGGUCCAACCUGU | 20 | 1982 |
| CCR5-1630 | + | UACUGCAAUUAUUCAGGCCA | 20 | 1983 |
| CCR5-1631 | + | UUAUUCAGGCCAAAGAAUUC | 20 | 1984 |
| CCR5-1632 | + | UAUUCAGGCCAAAGAAUUCC | 20 | 1985 |
| CCR5-1633 | + | AAGAAUUCCUGGAAGGUGUU | 20 | 1986 |
| CCR5-1634 | + | AGAAUUCCUGGAAGGUGUUC | 20 | 1987 |
| CCR5-1635 | + | AAUUCCUGGAAGGUGUUCAG | 20 | 1988 |
| CCR5-1636 | + | UCCUGGAAGGUGUUCAGGAG | 20 | 1989 |
| CCR5-1637 | + | UCAGGAGAAGGACAAUGUUG | 20 | 1990 |
| CCR5-1638 | + | CAGGAGAAGGACAAUGUUGU | 20 | 1991 |
| CCR5-1639 | + | AGGAGAAGGACAAUGUUGUA | 20 | 1992 |
| CCR5-1640 | + | GGACAAUGUUGUAGGGAGCC | 20 | 1993 |
| CCR5-1641 | + | CAAUGUUGUAGGGAGCCCAG | 20 | 1994 |
| CCR5-1642 | + | AUGUUGUAGGGAGCCCAGAA | 20 | 1995 |
| CCR5-1643 | + | GAAAAUAAACAAUCAUGAUG | 20 | 1996 |
| CCR5-1644 | + | CUCUUCUUCUCAUUUCGACA | 20 | 1997 |
| CCR5-1645 | + | UUCUCAUUUCGACACCGAAG | 20 | 1998 |
| CCR5-1646 | + | CGACACCGAAGCAGAGUUUU | 20 | 1999 |
| CCR5-1647 | + | AAGCAGAGUUUUUAGGAUUC | 20 | 2000 |
| CCR5-1648 | + | AUGACCAUGACAAGCAGCGG | 20 | 2001 |
| CCR5-1649 | + | AAGAUGACUAUCUUUAAUGU | 20 | 2002 |
| CCR5-1650 | + | AGAUGACUAUCUUUAAUGUC | 20 | 2003 |
| CCR5-1651 | + | UUAAUGUCUGGAAAUUCUUC | 20 | 2004 |
| CCR5-1652 | + | CCAGAAUUGAUACUGACUGU | 20 | 2005 |
| CCR5-1653 | + | CAGAAUUGAUACUGACUGUA | 20 | 2006 |
| CCR5-1654 | + | UGAUACUGACUGUAUGGAAA | 20 | 2007 |
| CCR5-1655 | + | AUACUGACUGUAUGGAAAAU | 20 | 2008 |
| CCR5-1656 | + | AAAUGAGAGCUGCAGGUGUA | 20 | 2009 |
| CCR5-1657 | + | GUGUAAUGAAGACCUUCUUU | 20 | 2010 |
| CCR5-1658 | + | GUAACUUUCACAUACAU | 17 | 2011 |
| CCR5-1659 | + | UGCAAAUACUAAGAUGU | 17 | 2012 |
| CCR5-1660 | + | AUGUCUUUGACUUGGCC | 17 | 2013 |
| CCR5-1661 | + | GUCUUUGACUUGGCCCA | 17 | 2014 |
| CCR5-1662 | + | UGACUUGGCCCAGAGGG | 17 | 2015 |
| CCR5-1663 | + | UGCACUCUCCACAACUU | 17 | 2016 |
| CCR5-1664 | + | CCACAACUUAAGAGCAA | 17 | 2017 |
| CCR5-1665 | + | CACAACUUAAGAGCAAA | 17 | 2018 |
| CCR5-1666 | + | UGCUCACCGUUCAUAUU | 17 | 2019 |
| CCR5-1667 | + | CCGUUCAUAUUCAGAGG | 17 | 2020 |
| CCR5-1668 | + | GUUCAUAUUCAGAGGCU | 17 | 2021 |
| CCR5-1669 | + | UCUGACACCUUCAUUCC | 17 | 2022 |
| CCR5-1670 | + | AGUAUGUGCACAAUCAU | 17 | 2023 |
| CCR5-1671 | + | UGCACAAUCAUAUGAGA | 17 | 2024 |
| CCR5-1672 | + | AAUCAUAUGAGACAGAA | 17 | 2025 |
| CCR5-1673 | + | AACCUCUCUCUCUCCCU | 17 | 2026 |
| CCR5-1674 | + | CUCUCUCUCCCUUUGAA | 17 | 2027 |
| CCR5-1675 | + | GAAUAUACCCAAACACU | 17 | 2028 |
| CCR5-1676 | + | AAUAUACCCAAACACUA | 17 | 2029 |
| CCR5-1677 | + | GGGGUAUAUUCAUUUCA | 17 | 2030 |
| CCR5-1678 | + | GGGUAUAUUCAUUUCAA | 17 | 2031 |
| CCR5-1679 | + | GGUAUAUUCAUUUCAAA | 17 | 2032 |
| CCR5-1680 | + | UAUAUUCAUUUCAAAGG | 17 | 2033 |
| CCR5-1681 | + | AUAUUCAUUUCAAAGGG | 17 | 2034 |
| CCR5-1682 | + | UAUUCAUUUCAAAGGGA | 17 | 2035 |
| CCR5-1683 | + | UUCAUUUCAAAGGGAGG | 17 | 2036 |
| CCR5-1684 | + | UGUUGCUUCUGGUUUGU | 17 | 2037 |
| CCR5-1685 | + | GUUGCUUCUGGUUUGUC | 17 | 2038 |
| CCR5-1686 | + | UGCUUCUGGUUUGUCUG | 17 | 2039 |
| CCR5-1687 | + | UUGUCUGGAGAAGGCAU | 17 | 2040 |
| CCR5-1688 | + | UGUCUGGAGAAGGCAUC | 17 | 2041 |
| CCR5-1689 | + | CCCCACCCCCAUUCAGU | 17 | 2042 |
| CCR5-1690 | + | CCCAUUCAGUCUGAAAU | 17 | 2043 |
| CCR5-1691 | + | CCAUUCAGUCUGAAAUA | 17 | 2044 |
| CCR5-1692 | + | UGGUAAAUUGUACUUUU | 17 | 2045 |
| CCR5-1693 | + | AGGCAGCUUAUUUCCAA | 17 | 2046 |
| CCR5-1694 | + | CUAUUGACGGUUAAAUG | 17 | 2047 |
| CCR5-1695 | + | ACCUACACUUGUGUGCA | 17 | 2048 |
| CCR5-1696 | + | AGGCUUCCCUCACCUCU | 17 | 2049 |
| CCR5-1697 | + | GGCUUCCCUCACCUCUA | 17 | 2050 |
| CCR5-1698 | + | UUUGCUCAGUGCUAUCC | 17 | 2051 |
| CCR5-1699 | + | CUCAGUGCUAUCCCUGA | 17 | 2052 |
| CCR5-1700 | + | UCCCUGAAUGAGUAACU | 17 | 2053 |
| CCR5-1701 | + | UAAGAGUUUGAUGCUUA | 17 | 2054 |
| CCR5-1702 | + | UGCCUGUGGUUGCCUCA | 17 | 2055 |
| CCR5-1703 | + | AAUCCUCCCAACAACCC | 17 | 2056 |
| CCR5-1704 | + | UCACCUAGAUCUCAUGU | 17 | 2057 |
| CCR5-1705 | + | UAGAUCUCAUGUGUGAG | 17 | 2058 |
| CCR5-1706 | + | AUAAAUCUAGUCUCCUC | 17 | 2059 |
| CCR5-1707 | + | UAAAUCUAGUCUCCUCC | 17 | 2060 |
| CCR5-1708 | + | ACCCCUCAGUAUUUCAG | 17 | 2061 |
| CCR5-1709 | + | CCCCUCAGUAUUUCAGC | 17 | 2062 |
| CCR5-1710 | + | UCAGUAUUUCAGCUGGG | 17 | 2063 |
| CCR5-1711 | + | CAGUAUUUCAGCUGGGA | 17 | 2064 |
| CCR5-1712 | + | AGUAUUUCAGCUGGGAU | 17 | 2065 |
| CCR5-1713 | + | AUUUCAGCUGGGAUGGG | 17 | 2066 |
| CCR5-1714 | + | UUUCAGCUGGGAUGGGA | 17 | 2067 |
| CCR5-1715 | + | GGAUGGGAAGGAAAUCU | 17 | 2068 |
| CCR5-1716 | + | AAGGAAAUCUAUGAAGU | 17 | 2069 |
| CCR5-1717 | + | GAAGUCAGAAGCAUUCA | 17 | 2070 |
| CCR5-1718 | + | AUUCAGUGAAAGACAGC | 17 | 2071 |
| CCR5-1719 | + | UUCAGUGAAAGACAGCC | 17 | 2072 |
| CCR5-1720 | + | GAAAGACAGCCUGGAGU | 17 | 2073 |
| CCR5-1721 | + | GACAGCCUGGAGUCUGG | 17 | 2074 |
| CCR5-1722 | + | GUGCUUGAUGUCUUUUC | 17 | 2075 |
| CCR5-1723 | + | GGGUUUCUCCAAUCUGC | 17 | 2076 |
| CCR5-1724 | + | CCAAUCUGCUUGAAGAC | 17 | 2077 |
| CCR5-1725 | + | CAAUCUGCUUGAAGACU | 17 | 2078 |
| CCR5-1726 | + | GCAUCCUCAUAUGCUGC | 17 | 2079 |
| CCR5-1727 | + | CCCUCCUUCCCAUCCUU | 17 | 2080 |
| CCR5-1728 | + | CUUCCCAUCCUCACGCC | 17 | 2081 |
| CCR5-1729 | + | UCACGCCUUGAGCUUAG | 17 | 2082 |
| CCR5-1730 | + | GCCAUCCUCACCCUGAC | 17 | 2083 |
| CCR5-1731 | + | CAUCCUCACCCUGACCU | 17 | 2084 |
| CCR5-1732 | + | UGACCCUCCUUUGGCCA | 17 | 2085 |
| CCR5-1733 | + | CCUUCUGCAACACCAAC | 17 | 2086 |
| CCR5-1734 | + | CUCAGCUCAUGACUUAG | 17 | 2087 |
| CCR5-1735 | + | UCAGCUCAUGACUUAGA | 17 | 2088 |
| CCR5-1736 | + | CCCAUGCAGUGCUUGCA | 17 | 2089 |
| CCR5-1737 | + | CAAAUUCCUUCUCAUUU | 17 | 2090 |
| CCR5-1738 | + | CGCCUGGUUCUAAGUCA | 17 | 2091 |
| CCR5-1739 | + | AACUUAUUAACCAUACC | 17 | 2092 |
| CCR5-1740 | + | ACUUAUUAACCAUACCU | 17 | 2093 |
| CCR5-1741 | + | UUAUUAACCAUACCUUG | 17 | 2094 |
| CCR5-1742 | + | UAUUAACCAUACCUUGG | 17 | 2095 |
| CCR5-1743 | + | AUUAACCAUACCUUGGA | 17 | 2096 |
| CCR5-1744 | + | UUAACCAUACCUUGGAG | 17 | 2097 |
| CCR5-1745 | + | UGGAGGGGAAAUCACAC | 17 | 2098 |
| CCR5-1746 | + | UAAAAAGUUGUACAUUU | 17 | 2099 |
| CCR5-1747 | + | UUCAGAUCACUAAACUC | 17 | 2100 |
| CCR5-1748 | + | CAAGAAUCAGCAAUUCU | 17 | 2101 |
| CCR5-1749 | + | UUCUUUUAAAUAUACAU | 17 | 2102 |
| CCR5-1750 | + | UCUUUUAAAUAUACAUA | 17 | 2103 |
| CCR5-1751 | + | AUAUACAUAAGGAACUU | 17 | 2104 |
| CCR5-1752 | + | UAUACAUAAGGAACUUU | 17 | 2105 |
| CCR5-1753 | + | CAUAAGGAACUUUCGGA | 17 | 2106 |
| CCR5-1754 | + | AAGGAACUUUCGGAGUG | 17 | 2107 |
| CCR5-1755 | + | AGGAACUUUCGGAGUGA | 17 | 2108 |
| CCR5-1756 | + | GGAACUUUCGGAGUGAA | 17 | 2109 |
| CCR5-1757 | + | AACUUUCGGAGUGAAGG | 17 | 2110 |
| CCR5-1758 | + | UCAAUAACUUGAUGCAU | 17 | 2111 |
| CCR5-1759 | + | AUAACUUGAUGCAUGUG | 17 | 2112 |
| CCR5-1760 | + | UAACUUGAUGCAUGUGA | 17 | 2113 |
| CCR5-1761 | + | AACUUGAUGCAUGUGAA | 17 | 2114 |
| CCR5-1762 | + | ACUUGAUGCAUGUGAAG | 17 | 2115 |
| CCR5-1763 | + | UUGGCUUUCUAUAAUUG | 17 | 2116 |
| CCR5-1764 | + | AAACAGAUGCCAAAUAA | 17 | 2117 |
| CCR5-1765 | + | AGAUGCCAAAUAAAUGG | 17 | 2118 |
| CCR5-1766 | + | CCCAGCCCAGGCUGUGU | 17 | 2119 |
| CCR5-1767 | + | CAUGUGCACAACUCUGA | 17 | 2120 |
| CCR5-1768 | + | CUGGGUCACCAGCCCAC | 17 | 2121 |
| CCR5-1769 | + | AUAUUUCCUGCUCCCCA | 17 | 2122 |
| CCR5-1770 | + | UCCUGCUCCCCAGUGGA | 17 | 2123 |
| CCR5-1771 | + | AGUGGAUCGGGUGUAAA | 17 | 2124 |
| CCR5-1772 | + | AAACUGAGCUUGCUCGC | 17 | 2125 |
| CCR5-1773 | + | AACUGAGCUUGCUCGCU | 17 | 2126 |
| CCR5-1774 | + | ACUGAGCUUGCUCGCUC | 17 | 2127 |
| CCR5-1775 | + | CGCUCGGGAGCCUCUUG | 17 | 2128 |
| CCR5-1776 | + | GCUCGGGAGCCUCUUGC | 17 | 2129 |
| CCR5-1777 | + | AGCCUCUUGCUGGAAAA | 17 | 2130 |
| CCR5-1778 | + | AAAUAGAACAGCAUUUG | 17 | 2131 |
| CCR5-1779 | + | CGUUUGGCAAUGUGCUU | 17 | 2132 |
| CCR5-1780 | + | GUUUGGCAAUGUGCUUU | 17 | 2133 |
| CCR5-1781 | + | UGGCAAUGUGCUUUUGG | 17 | 2134 |
| CCR5-1782 | + | GCUUUUGGAAGAAGACU | 17 | 2135 |
| CCR5-1783 | + | AGACUAAGAGGUAGUUU | 17 | 2136 |
| CCR5-1784 | + | CGACAAAGGCAUAGAUG | 17 | 2137 |
| CCR5-1785 | + | GACAAAGGCAUAGAUGA | 17 | 2138 |
| CCR5-1786 | + | CAGCAGUGCGUCAUCCC | 17 | 2139 |
| CCR5-1787 | + | AGCUUGGUCCAACCUGU | 17 | 2140 |
| CCR5-1788 | + | UGCAAUUAUUCAGGCCA | 17 | 2141 |
| CCR5-1789 | + | UUCAGGCCAAAGAAUUC | 17 | 2142 |
| CCR5-1790 | + | UCAGGCCAAAGAAUUCC | 17 | 2143 |
| CCR5-1791 | + | AAUUCCUGGAAGGUGUU | 17 | 2144 |
| CCR5-1792 | + | AUUCCUGGAAGGUGUUC | 17 | 2145 |
| CCR5-1793 | + | UCCUGGAAGGUGUUCAG | 17 | 2146 |
| CCR5-1794 | + | UGGAAGGUGUUCAGGAG | 17 | 2147 |
| CCR5-1795 | + | GGAGAAGGACAAUGUUG | 17 | 2148 |
| CCR5-1796 | + | GAGAAGGACAAUGUUGU | 17 | 2149 |
| CCR5-1797 | + | AGAAGGACAAUGUUGUA | 17 | 2150 |
| CCR5-1798 | + | CAAUGUUGUAGGGAGCC | 17 | 2151 |
| CCR5-1799 | + | UGUUGUAGGGAGCCCAG | 17 | 2152 |
| CCR5-1800 | + | UUGUAGGGAGCCCAGAA | 17 | 2153 |
| CCR5-1801 | + | AAUAAACAAUCAUGAUG | 17 | 2154 |
| CCR5-1802 | + | UUCUUCUCAUUUCGACA | 17 | 2155 |
| CCR5-1803 | + | UCAUUUCGACACCGAAG | 17 | 2156 |
| CCR5-1804 | + | CACCGAAGCAGAGUUUU | 17 | 2157 |
| CCR5-1805 | + | CAGAGUUUUUAGGAUUC | 17 | 2158 |
| CCR5-1806 | + | ACCAUGACAAGCAGCGG | 17 | 2159 |
| CCR5-1807 | + | AUGACUAUCUUUAAUGU | 17 | 2160 |
| CCR5-1808 | + | UGACUAUCUUUAAUGUC | 17 | 2161 |
| CCR5-1809 | + | AUGUCUGGAAAUUCUUC | 17 | 2162 |
| CCR5-1810 | + | GAAUUGAUACUGACUGU | 17 | 2163 |
| CCR5-1811 | + | AAUUGAUACUGACUGUA | 17 | 2164 |
| CCR5-1812 | + | UACUGACUGUAUGGAAA | 17 | 2165 |
| CCR5-1813 | + | CUGACUGUAUGGAAAAU | 17 | 2166 |
| CCR5-1814 | + | UGAGAGCUGCAGGUGUA | 17 | 2167 |
| CCR5-1815 | + | UAAUGAAGACCUUCUUU | 17 | 2168 |

Table 1F provides exemplary targeting domains for knocking out the CCR5 gene. In an embodiment, the targeting domain is the exact complement of the target domain. Any of the targeting domains in the table can be used with an N. meningitides Cas9 molecule that gives double stranded cleavage. Any of the targeting domains in the table can be used with an N. meningitides Cas9 single-stranded break nucleases (nickases). In an embodiment, dual targeting is used to create two nicks.

TABLE 1F

| | | | Target | |
|---|---|---|---|---|
| gRNA | DNA | | Site | SEQ ID |
| Name | Strand | Targeting Domain | Length | NO |
| CCR5-1816 | + | AUGGACGACAGCCAGGUACC | 20 | 2169 |
| CCR5-1817 | + | GAUUGUCAGGAGGAUGAUGA | 20 | 2170 |
| CCR5-1818 | + | GAGCGGAGGCAGGAGGCGGG | 20 | 2171 |
| CCR5-1819 | + | GCGGGCUGCGAUUUGCUUCA | 20 | 2172 |
| CCR5-1820 | + | CGAUGUAUAAUAAUUGAUGU | 20 | 2173 |
| CCR5-1821 | + | GACGACAGCCAGGUACC | 17 | 2174 |
| CCR5-1822 | + | UGUCAGGAGGAUGAUGA | 17 | 2175 |
| CCR5-1823 | + | CGGAGGCAGGAGGCGGG | 17 | 2176 |
| CCR5-1824 | + | GGCUGCGAUUUGCUUCA | 17 | 2177 |
| CCR5-1825 | + | UGUAUAAUAAUUGAUGU | 17 | 2178 |
| CCR5-1826 | − | UGUGAGGCUUAUCUUCACCA | 20 | 2179 |
| CCR5-1827 | − | AAGUUACUGUUAUAGAGGGU | 20 | 2180 |
| CCR5-1828 | − | UUUAUUUGGCAUCUGUUUAA | 20 | 2181 |
| CCR5-1829 | − | AAAAGAAAGCCUCAGAGAAU | 20 | 2182 |
| CCR5-1830 | − | UAUGGGGAGAAAAGACAUGA | 20 | 2183 |
| CCR5-1831 | − | AAAGAAAUGACACUUUUCAU | 20 | 2184 |
| CCR5-1832 | − | UGCAGAGUCAGCAGAACUGG | 20 | 2185 |
| CCR5-1833 | − | GAGAGAAUCCCUAGUCUUCA | 20 | 2186 |
| CCR5-1834 | − | GAGGUUUAGGUCAAGAAGAA | 20 | 2187 |
| CCR5-1835 | − | UCACUGAAUGCUUCUGACUU | 20 | 2188 |
| CCR5-1836 | − | UGAGGGGUCUCCAGGAGGAG | 20 | 2189 |
| CCR5-1837 | − | GCUCACACAUGAGAUCUAGG | 20 | 2190 |
| CCR5-1838 | − | ACACAUGAGAUCUAGGUGAG | 20 | 2191 |
| CCR5-1839 | − | AGUCAUUUCAUGGGUUGUUG | 20 | 2192 |
| CCR5-1840 | − | GUUUUUUUCUGUUCUGUCUC | 20 | 2193 |
| CCR5-1841 | − | GAGGCUUAUCUUCACCA | 17 | 2194 |
| CCR5-1842 | − | UUACUGUUAUAGAGGGU | 17 | 2195 |
| CCR5-1843 | − | AUUUGGCAUCUGUUUAA | 17 | 2196 |
| CCR5-1844 | − | AGAAAGCCUCAGAGAAU | 17 | 2197 |
| CCR5-1845 | − | GGGGAGAAAAGACAUGA | 17 | 2198 |
| CCR5-1846 | − | GAAAUGACACUUUUCAU | 17 | 2199 |
| CCR5-1847 | − | AGAGUCAGCAGAACUGG | 17 | 2200 |
| CCR5-1848 | − | AGAAUCCCUAGUCUUCA | 17 | 2201 |
| CCR5-1849 | − | GUUUAGGUCAAGAAGAA | 17 | 2202 |
| CCR5-1850 | − | CUGAAUGCUUCUGACUU | 17 | 2203 |
| CCR5-1851 | − | GGGGUCUCCAGGAGGAG | 17 | 2204 |
| CCR5-1852 | − | CACACAUGAGAUCUAGG | 17 | 2205 |
| CCR5-1853 | − | CAUGAGAUCUAGGUGAG | 17 | 2206 |
| CCR5-1854 | − | CAUUUCAUGGGUUGUUG | 17 | 2207 |
| CCR5-1855 | − | UUUUUCUGUUCUGUCUC | 17 | 2208 |
| CCR5-1856 | + | UUCAUUUCAAAGGGAGGGAG | 20 | 2209 |
| CCR5-1857 | + | UCUCCAAUCUGCUUGAAGAC | 20 | 2210 |
| CCR5-1858 | + | UGCUAUUUUUCAUCAACAUA | 20 | 2211 |
| CCR5-1859 | + | UCGACACCGAAGCAGAGUUU | 20 | 2212 |
| CCR5-1860 | + | AUUUCAAAGGGAGGGAG | 17 | 2213 |
| CCR5-1861 | + | CCAAUCUGCUUGAAGAC | 17 | 2214 |
| CCR5-1862 | + | UAUUUUUCAUCAACAUA | 17 | 2215 |
| CCR5-1863 | + | ACACCGAAGCAGAGUUU | 17 | 2216 |

Table 2A provides exemplary targeting domains for knocking out the CCR5 gene selected according to the first tier parameters. The targeting domains bind within the first 500 bp of the coding sequence (e.g., within 500 bp downstream from the start codon) and have a high level of orthogonality. It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 2A
1st Tier

| | | | Target | |
|---|---|---|---|---|
| gRNA | DNA | | Site | SEQ ID |
| Name | Strand | Targeting Domain | Length | NO |
| CCR5-115 | − | ACUAUGCUGCCGCCCAG | 17 | 4343 |
| CCR5-121 | − | UCCUCCUGACAAUCGAU | 17 | 4344 |
| CCR5-116 | − | CUAUGCUGCCGCCCAGU | 17 | 4345 |
| CCR5-3 | − | GCCGCCCAGUGGGACUU | 17 | 4346 |
| CCR5-53 | − | UUGACAGGGCUCUAUUUUAU | 20 | 4347 |
| CCR5-75 | − | UCACUAUGCUGCCGCCCAGU | 20 | 4348 |

Table 2B provides exemplary targeting domains for knocking out the CCR5 gene selected according to the second tier parameters. The targeting domains bind within the first 500 bp of the coding sequence (e.g., within 500 bp downstream from the start codon). It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 2B
2nd Tier

| | | | Target | |
|---|---|---|---|---|
| gRNA | DNA | | Site | SEQ ID |
| Name | Strand | Targeting Domain | Length | NO |
| CCR5-111 | − | UCCUGAUAAACUGCAAA | 17 | 4349 |
| CCR5-135 | + | ACUUGUCACCACCCCAA | 17 | 4350 |
| CCR5-4 | + | GCAUAGUGAGCCCAGAA | 17 | 4351 |
| CCR5-1864 | − | CUUUUUAUUUAUGCACA | 17 | 4352 |
| CCR5-118 | − | UGUGUCAACUCUUGACA | 17 | 4353 |
| CCR5-151 | + | UUAAAGCAAACACAGCA | 17 | 4354 |
| CCR5-132 | + | ACAUUGAUUUUUUGGCA | 17 | 4355 |
| CCR5-1865 | − | ACCAGAUCUCAAAAAGA | 17 | 4356 |
| CCR5-1866 | − | CACAGGGUGGAACAAGA | 17 | 4357 |
| CCR5-136 | + | AGAAGGGGACAGUAAGA | 17 | 4358 |
| CCR5-139 | + | AGCAUAGUGAGCCCAGA | 17 | 4359 |
| CCR5-5 | + | GAAAAACAGGUCAGAGA | 17 | 4360 |
| CCR5-123 | − | UGCUUUAAAAGCCAGGA | 17 | 4361 |
| CCR5-144 | + | CAGUAAGAAGGAAAAAC | 17 | 4362 |
| CCR5-148 | + | UAUUUCCAAAGUCCCAC | 17 | 4363 |
| CCR5-1867 | − | ACUUUUUAUUUAUGCAC | 17 | 4364 |
| CCR5-1 | − | GCCUCCGCUCUACUCAC | 17 | 4365 |
| CCR5-52 | − | AUGUGUCAACUCUUGAC | 17 | 4366 |
| CCR5-112 | − | CAUCUACCUGCUCAACC | 17 | 4367 |
| CCR5-10 | − | GACAAUCGAUAGGUACC | 17 | 4368 |
| CCR5-129 | − | GUGUUUGCGUCUCUCCC | 17 | 4369 |
| CCR5-122 | − | UGUUUGCUUUAAAAGCC | 17 | 4370 |
| CCR5-143 | + | CAGCAUGGACGACAGCC | 17 | 4371 |
| CCR5-131 | + | ACAGGUCAGAGAUGGCC | 17 | 4372 |
| CCR5-146 | + | CCCAAAGGUGACCGUCC | 17 | 4373 |
| CCR5-1868 | + | CUGGUAAAGAUGAUUCC | 17 | 4374 |
| CCR5-138 | + | AGAUGGCCAGGUUGAGC | 17 | 4375 |
| CCR5-8 | + | GAGCGGAGGCAGGAGGC | 17 | 4376 |
| CCR5-7 | + | GUGAGUAGAGCGGAGGC | 17 | 4377 |
| CCR5-64 | + | CACAUUGAUUUUUUGGC | 17 | 4378 |
| CCR5-110 | − | UUUUGUGGGCAACAUGC | 17 | 4379 |
| CCR5-1869 | + | ACCUUCUUUUUGAGAUC | 17 | 4380 |
| CCR5-6 | + | GCCUUUUGCAGUUUAUC | 17 | 4381 |
| CCR5-120 | − | UUUAUAGGCUUCUUCUC | 17 | 4382 |
| CCR5-14 | + | GGUACCUAUCGAUUGUC | 17 | 4383 |
| CCR5-113 | − | UUCUUACUGUCCCCUUC | 17 | 4384 |
| CCR5-145 | + | CAUAGUGAGCCCAGAAG | 17 | 4385 |
| CCR5-130 | + | AACACCAGUGAGUAGAG | 17 | 4386 |
| CCR5-65 | + | AGUAGAGCGGAGGCAGG | 17 | 4387 |
| CCR5-134 | + | ACCUAUCGAUUGUCAGG | 17 | 4388 |
| CCR5-137 | + | AGAGCGGAGGCAGGAGG | 17 | 4389 |
| CCR5-133 | + | ACCAGUGAGUAGAGCGG | 17 | 4390 |
| CCR5-1870 | − | UUUAUUUAUGCACAGGG | 17 | 4391 |
| CCR5-12 | − | GACGGUCACCUUUGGGG | 17 | 4392 |
| CCR5-149 | + | UCCAAAGUCCCACUGGG | 17 | 4393 |
| CCR5-127 | − | AAGUGUGAUCACUUGGG | 17 | 4394 |
| CCR5-128 | − | UGUGAUCACUUGGGUGG | 17 | 4395 |
| CCR5-150 | + | UGCAGUUUAUCAGGAUG | 17 | 4396 |
| CCR5-125 | − | CAGGACGGUCACCUUUG | 17 | 4397 |
| CCR5-2 | − | GUUCAUCUUUGGUUUUG | 17 | 4398 |
| CCR5-107 | − | CAUCAAUUAUUAUACAU | 17 | 4399 |
| CCR5-147 | + | UAAUUGAUGUCAUAGAU | 17 | 4400 |
| CCR5-119 | − | ACAGGGCUCUAUUUUAU | 17 | 4401 |
| CCR5-141 | + | AUUUCCAAAGUCCCACU | 17 | 4402 |
| CCR5-126 | − | UGACAAGUGUGAUCACU | 17 | 4403 |
| CCR5-1871 | + | UGGUAAAGAUGAUUCCU | 17 | 4404 |
| CCR5-114 | − | UCUUACUGUCCCCUUCU | 17 | 4405 |
| CCR5-109 | − | UUCAUCUUUGGUUUUGU | 17 | 4406 |
| CCR5-13 | − | GACAAGUGUGAUCACUU | 17 | 4407 |
| CCR5-11 | − | GCCAGGACGGUCACCUU | 17 | 4408 |
| CCR5-108 | − | UCACUGGUGUUCAUCUU | 17 | 4409 |
| CCR5-124 | − | CCAGGACGGUCACCUUU | 17 | 4410 |
| CCR5-9 | + | GCUUCACAUUGAUUUUU | 17 | 4411 |
| CCR5-70 | − | UCAUCCUGAUAAACUGCAAA | 20 | 4412 |
| CCR5-94 | + | CACACUUGUCACCACCCCAA | 20 | 4413 |
| CCR5-47 | + | GCAGCAUAGUGAGCCCAGAA | 20 | 4414 |
| CCR5-76 | − | CAAUGUGUCAACUCUUGACA | 20 | 4415 |
| CCR5-100 | + | CUUUUAAAGCAAACACAGCA | 20 | 4416 |
| CCR5-103 | + | UUCACAUUGAUUUUUUGGCA | 20 | 4417 |
| CCR5-1872 | − | UUUACCAGAUCUCAAAAAGA | 20 | 4418 |
| CCR5-1873 | − | AUGCACAGGGUGGAACAAGA | 20 | 4419 |
| CCR5-99 | + | CCCAGAAGGGGACAGUAAGA | 20 | 4420 |
| CCR5-46 | + | GGCAGCAUAGUGAGCCCAGA | 20 | 4421 |
| CCR5-89 | + | AAGGAAAAACAGGUCAGAGA | 20 | 4422 |
| CCR5-79 | − | GUUUGCUUUAAAAGCCAGGA | 20 | 4423 |
| CCR5-48 | + | GGACAGUAAGAAGGAAAAAC | 20 | 4424 |
| CCR5-104 | + | UUGUAUUUCCAAAGUCCCAC | 20 | 4425 |
| CCR5-66 | − | CCUGCCUCCGCUCUACUCAC | 20 | 4426 |
| CCR5-51 | − | ACAAUGUGUCAACUCUUGAC | 20 | 4427 |
| CCR5-71 | − | UGACAUCUACCUGCUCAACC | 20 | 4428 |
| CCR5-57 | − | CCUGACAAUCGAUAGGUACC | 20 | 4429 |
| CCR5-59 | − | GCUGUGUUUGCGUCUCUCCC | 20 | 4430 |
| CCR5-78 | − | CUGUGUUUGCUUUAAAAGCC | 20 | 4431 |
| CCR5-90 | + | ACACAGCAUGGACGACAGCC | 20 | 4432 |
| CCR5-87 | + | AAAACAGGUCAGAGAUGGCC | 20 | 4433 |
| CCR5-95 | + | CACCCCAAAGGUGACCGUCC | 20 | 4434 |
| CCR5-1874 | + | GAUCUGGUAAAGAUGAUUCC | 20 | 4435 |
| CCR5-96 | + | CAGAGAUGGCCAGGUUGAGC | 20 | 4436 |
| CCR5-50 | + | GUAGAGCGGAGGCAGGAGGC | 20 | 4437 |
| CCR5-98 | + | CCAGUGAGUAGAGCGGAGGC | 20 | 4438 |
| CCR5-63 | + | CUUCACAUUGAUUUUUUGGC | 20 | 4439 |
| CCR5-69 | − | UGGUUUUGUGGGCAACAUGC | 20 | 4440 |
| CCR5-1875 | + | AAGACCUUCUUUUUGAGAUC | 20 | 4441 |
| CCR5-62 | + | UCAGCCUUUUGCAGUUUAUC | 20 | 4442 |
| CCR5-77 | − | UAUUUUAUAGGCUUCUUCUC | 20 | 4443 |
| CCR5-60 | + | CCAGGUACCUAUCGAUUGUC | 20 | 4444 |
| CCR5-72 | − | UCCUUCUUACUGUCCCCUUC | 20 | 4445 |
| CCR5-97 | + | CAGCAUAGUGAGCCCAGAAG | 20 | 4446 |
| CCR5-74 | − | CUCACUAUGCUGCCGCCCAG | 20 | 4447 |
| CCR5-92 | + | AUGAACACCAGUGAGUAGAG | 20 | 4448 |
| CCR5-49 | + | GUGAGUAGAGCGGAGGCAGG | 20 | 4449 |
| CCR5-45 | + | GGUACCUAUCGAUUGUCAGG | 20 | 4450 |
| CCR5-91 | + | AGUAGAGCGGAGGCAGGAGG | 20 | 4451 |
| CCR5-88 | + | AACACCAGUGAGUAGAGCGG | 20 | 4452 |
| CCR5-1876 | − | CUUUUUAUUUAUGCACAGGG | 20 | 4453 |
| CCR5-83 | − | CAGGACGGUCACCUUUGGGG | 20 | 4454 |
| CCR5-93 | + | AUUUCCAAAGUCCCACUGGG | 20 | 4455 |
| CCR5-85 | − | GACAAGUGUGAUCACUUGGG | 20 | 4456 |
| CCR5-86 | − | AAGUGUGAUCACUUGGGUGG | 20 | 4457 |
| CCR5-106 | + | UUUUGCAGUUUAUCAGGAUG | 20 | 4458 |
| CCR5-82 | − | AGCCAGGACGGUCACCUUUG | 20 | 4459 |
| CCR5-41 | − | GGUGUUCAUCUUUGGUUUUG | 20 | 4460 |
| CCR5-67 | − | UGACAUCAAUUAUUAUACAU | 20 | 4461 |
| CCR5-101 | + | UAAUAAUUGAUGUCAUAGAU | 20 | 4462 |
| CCR5-55 | − | UCAUCCUCCUGACAAUCGAU | 20 | 4463 |
| CCR5-102 | + | UGUAUUUCCAAAGUCCCACU | 20 | 4464 |
| CCR5-84 | − | UGGUGACAAGUGUGAUCACU | 20 | 4465 |
| CCR5-1877 | + | AUCUGGUAAAGAUGAUUCCU | 20 | 4466 |
| CCR5-73 | − | CCUUCUUACUGUCCCCUUCU | 20 | 4467 |
| CCR5-42 | − | GUGUUCAUCUUUGGUUUUGU | 20 | 4468 |
| CCR5-58 | − | GGUGACAAGUGUGAUCACUU | 20 | 4469 |
| CCR5-43 | − | GCUGCCGCCCAGUGGGACUU | 20 | 4470 |
| CCR5-80 | − | AAAGCCAGGACGGUCACCUU | 20 | 4471 |
| CCR5-68 | − | UACUCACUGGUGUUCAUCUU | 20 | 4472 |
| CCR5-81 | − | AAGCCAGGACGGUCACCUUU | 20 | 4473 |
| CCR5-105 | + | UUUGCUUCACAUUGAUUUUU | 20 | 4474 |

Table 2C provides exemplary targeting domains for knocking out the CCR5 gene selected according to the third tier parameters. The targeting domains fall in the coding sequence of the gene, downstream of the first 500 bp of coding sequence (e.g., anywhere from +500 (relative to the start codon) to the stop codon of the gene). It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 2C
3rd Tier

| | | | Target | |
|---|---|---|---|---|
| gRNA | DNA | | Site | SEQ ID |
| Name | Strand | Targeting Domain | Length | NO |
| CCR5-793 | + | GAACUUCUCCCCGACAA | 17 | 4475 |
| CCR5-382 | − | UGAGAAGAAGAGGCACA | 17 | 4476 |
| CCR5-403 | − | UCUGUGGGCUUGUGACA | 17 | 4477 |
| CCR5-376 | − | CCUGCCGCUGCUUGUCA | 17 | 4478 |
| CCR5-1865 | − | ACCAGAUCUCAAAAAGA | 17 | 4479 |
| CCR5-802 | + | GGAAGGUGUUCAGGAGA | 17 | 4480 |
| CCR5-800 | + | GCCAAAGAAUUCCUGGA | 17 | 4481 |
| CCR5-805 | + | AAAAUAAACAAUCAUGA | 17 | 4482 |
| CCR5-794 | + | GACAAAGGCAUAGAUGA | 17 | 4483 |
| CCR5-810 | + | AAUUGAUACUGACUGUA | 17 | 4484 |
| CCR5-804 | + | AGAAGGACAAUGUUGUA | 17 | 4485 |
| CCR5-388 | − | AUUGCAGUAGCUCUAAC | 17 | 4486 |
| CCR5-397 | − | GUUUACACCCGAUCCAC | 17 | 4487 |
| CCR5-381 | − | AUGAGAAGAAGAGGCAC | 17 | 4488 |
| CCR5-799 | + | UCAGGCCAAAGAAUUCC | 17 | 4489 |
| CCR5-1868 | + | CUGGUAAAGAUGAUUCC | 17 | 4490 |
| CCR5-386 | − | UCUCCUGAACACCUUCC | 17 | 4491 |
| CCR5-400 | − | CCGAUCCACUGGGGAGC | 17 | 4492 |
| CCR5-808 | + | CCAUGACAAGCAGCGGC | 17 | 4493 |
| CCR5-375 | − | GAUAGUCAUCUUGGGGC | 17 | 4494 |
| CCR5-406 | − | CACGGACUCAAGUGGGC | 17 | 4495 |
| CCR5-390 | − | GUUGGACCAAGCUAUGC | 17 | 4496 |
| CCR5-811 | + | UGGAAAAUGAGAGCUGC | 17 | 4497 |
| CCR5-789 | + | GCUCGGGAGCCUCUUGC | 17 | 4498 |
| CCR5-1869 | + | ACCUUCUUUUUGAGAUC | 17 | 4499 |
| CCR5-786 | + | CUGCUCCCCAGUGGAUC | 17 | 4500 |
| CCR5-378 | − | AUGGUCAUCUGCUACUC | 17 | 4501 |
| CCR5-788 | + | ACUGAGCUUGCUCGCUC | 17 | 4502 |
| CCR5-809 | + | UGACUAUCUUUAAUGUC | 17 | 4503 |
| CCR5-394 | − | UCAUCUAUGCCUUUGUC | 17 | 4504 |
| CCR5-371 | − | ACAGUCAGUAUCAAUUC | 17 | 4505 |
| CCR5-798 | + | AGCUACUGCAAUUAUUC | 17 | 4506 |
| CCR5-384 | − | UUGUUUAUUUUCUCUUC | 17 | 4507 |
| CCR5-801 | + | AUUCCUGGAAGGUGUUC | 17 | 4508 |
| CCR5-396 | − | UUCUAUUUUCCAGCAAG | 17 | 4509 |
| CCR5-404 | − | UGUGACACGGACUCAAG | 17 | 4510 |
| CCR5-380 | − | GUCGAAAUGAGAAGAAG | 17 | 4511 |
| CCR5-792 | + | UUUGGAAGAAGACUAAG | 17 | 4512 |
| CCR5-784 | + | UAUUUCCUGCUCCCCAG | 17 | 4513 |
| CCR5-807 | + | AUGACCAUGACAAGCAG | 17 | 4514 |
| CCR5-395 | − | CAUCUAUGCCUUUGUCG | 17 | 4515 |
| CCR5-796 | + | CAAAGGCAUAGAUGAUG | 17 | 4516 |
| CCR5-399 | − | UUACACCCGAUCCACUG | 17 | 4517 |
| CCR5-401 | − | GGAGCAGGAAAUAUCUG | 17 | 4518 |
| CCR5-383 | − | AGAGGCACAGGGCUGUG | 17 | 4519 |
| CCR5-374 | − | UAAAGAUAGUCAUCUUG | 17 | 4520 |
| CCR5-785 | + | CCUGCUCCCCAGUGGAU | 17 | 4521 |
| CCR5-795 | + | ACAAAGGCAUAGAUGAU | 17 | 4522 |
| CCR5-398 | − | UUUACACCCGAUCCACU | 17 | 4523 |
| CCR5-377 | − | CAUGGUCAUCUGCUACU | 17 | 4524 |
| CCR5-1871 | + | UGGUAAAGAUGAUUCCU | 17 | 4525 |
| CCR5-797 | + | CUGUCACCUGCAUAGCU | 17 | 4526 |
| CCR5-787 | + | AACUGAGCUUGCUCGCU | 17 | 4527 |
| CCR5-372 | − | AUUAAAGAUAGUCAUCU | 17 | 4528 |
| CCR5-391 | − | CAGGUGACAGAGACUCU | 17 | 4529 |
| CCR5-385 | − | UGUUUAUUUUCUCUUCU | 17 | 4530 |
| CCR5-405 | − | GUGACACGGACUCAAGU | 17 | 4531 |
| CCR5-389 | − | CAGUAGCUCUAACAGGU | 17 | 4532 |
| CCR5-402 | − | GAGCAGGAAAUAUCUGU | 17 | 4533 |
| CCR5-803 | + | GAGAAGGACAAUGUUGU | 17 | 4534 |
| CCR5-393 | − | AUCAUCUAUGCCUUUGU | 17 | 4535 |
| CCR5-379 | − | UCCUAAAAACUCUGCUU | 17 | 4536 |
| CCR5-373 | − | UUAAAGAUAGUCAUCUU | 17 | 4537 |
| CCR5-392 | − | AGGUGACAGAGACUCUU | 17 | 4538 |
| CCR5-387 | − | ACCUUCCAGGAAUUCUU | 17 | 4539 |
| CCR5-790 | + | GCAUUUGCAGAAGCGUU | 17 | 4540 |
| CCR5-791 | + | GUUUGGCAAUGUGCUUU | 17 | 4541 |
| CCR5-806 | + | ACCGAAGCAGAGUUUUU | 17 | 4542 |
| CCR5-682 | + | UCUGAACUUCUCCCCGACAA | 20 | 4543 |
| CCR5-163 | − | AAAUGAGAAGAAGAGGCACA | 20 | 4544 |
| CCR5-184 | − | AUAUCUGUGGGCUUGUGACA | 20 | 4545 |
| CCR5-157 | − | GGUCCUGCCGCUGCUUGUCA | 20 | 4546 |
| CCR5-1872 | − | UUUACCAGAUCUCAAAAAGA | 20 | 4547 |
| CCR5-691 | + | CCUGGAAGGUGUUCAGGAGA | 20 | 4548 |
| CCR5-689 | + | CAGGCCAAAGAAUUCCUGGA | 20 | 4549 |
| CCR5-694 | + | GAGAAAAUAAACAAUCAUGA | 20 | 4550 |
| CCR5-683 | + | CCCGACAAAGGCAUAGAUGA | 20 | 4551 |
| CCR5-699 | + | CAGAAUUGAUACUGACUGUA | 20 | 4552 |
| CCR5-693 | + | AGGAGAAGGACAAUGUUGUA | 20 | 4553 |
| CCR5-169 | − | AUAAUUGCAGUAGCUCUAAC | 20 | 4554 |
| CCR5-178 | − | UCAGUUUACACCCGAUCCAC | 20 | 4555 |
| CCR5-162 | − | GAAAUGAGAAGAAGAGGCAC | 20 | 4556 |
| CCR5-688 | + | UAUUCAGGCCAAAGAAUUCC | 20 | 4557 |
| CCR5-1874 | + | GAUCUGGUAAAGAUGAUUCC | 20 | 4558 |
| CCR5-167 | − | CCUUCUCCUGAACACCUUCC | 20 | 4559 |
| CCR5-181 | − | CACCCGAUCCACUGGGGAGC | 20 | 4560 |
| CCR5-697 | + | UGACCAUGACAAGCAGCGGC | 20 | 4561 |
| CCR5-156 | − | AAAGAUAGUCAUCUUGGGGC | 20 | 4562 |
| CCR5-187 | − | UGACACGGACUCAAGUGGGC | 20 | 4563 |
| CCR5-171 | − | CAGGUUGGACCAAGCUAUGC | 20 | 4564 |
| CCR5-700 | + | GUAUGGAAAAUGAGAGCUGC | 20 | 4565 |
| CCR5-678 | + | CUCGCUCGGGAGCCUCUUGC | 20 | 4566 |
| CCR5-1875 | + | AAGACCUUCUUUUUGAGAUC | 20 | 4567 |
| CCR5-675 | + | UUCCUGCUCCCCAGUGGAUC | 20 | 4568 |
| CCR5-159 | − | GUCAUGGUCAUCUGCUACUC | 20 | 4569 |
| CCR5-677 | + | UAAACUGAGCUUGCUCGCUC | 20 | 4570 |
| CCR5-698 | + | AGAUGACUAUCUUUAAUGUC | 20 | 4571 |
| CCR5-175 | − | CCAUCAUCUAUGCCUUUGUC | 20 | 4572 |
| CCR5-152 | − | CAUACAGUCAGUAUCAAUUC | 20 | 4573 |
| CCR5-687 | + | UAGAGCUACUGCAAUUAUUC | 20 | 4574 |
| CCR5-165 | − | UGAUUGUUUAUUUUCUCUUC | 20 | 4575 |
| CCR5-690 | + | AGAAUUCCUGGAAGGUGUUC | 20 | 4576 |
| CCR5-177 | − | CUGUUCUAUUUUCCAGCAAG | 20 | 4577 |
| CCR5-185 | − | GCUUGUGACACGGACUCAAG | 20 | 4578 |
| CCR5-161 | − | GGUGUCGAAAUGAGAAGAAG | 20 | 4579 |
| CCR5-681 | + | GCUUUUGGAAGAAGACUAAG | 20 | 4580 |
| CCR5-673 | + | AGAUAUUUCCUGCUCCCCAG | 20 | 4581 |
| CCR5-696 | + | CAGAUGACCAUGACAAGCAG | 20 | 4582 |
| CCR5-176 | − | CAUCAUCUAUGCCUUUGUCG | 20 | 4583 |
| CCR5-685 | + | CGACAAAGGCAUAGAUGAUG | 20 | 4584 |
| CCR5-180 | − | AGUUUACACCCGAUCCACUG | 20 | 4585 |
| CCR5-182 | − | UGGGGAGCAGGAAAUAUCUG | 20 | 4586 |
| CCR5-164 | − | AGAAGAGGCACAGGGCUGUG | 20 | 4587 |
| CCR5-155 | − | CAUUAAAGAUAGUCAUCUUG | 20 | 4588 |
| CCR5-674 | + | UUUCCUGCUCCCCAGUGGAU | 20 | 4589 |
| CCR5-684 | + | CCGACAAAGGCAUAGAUGAU | 20 | 4590 |
| CCR5-179 | − | CAGUUUACACCCGAUCCACU | 20 | 4591 |
| CCR5-158 | − | UGUCAUGGUCAUCUGCUACU | 20 | 4592 |
| CCR5-1877 | + | AUCUGGUAAAGAUGAUUCCU | 20 | 4593 |
| CCR5-686 | + | UCUCUGUCACCUGCAUAGCU | 20 | 4594 |
| CCR5-676 | + | GUAAACUGAGCUUGCUCGCU | 20 | 4595 |
| CCR5-153 | − | GACAUUAAAGAUAGUCAUCU | 20 | 4596 |
| CCR5-172 | − | AUGCAGGUGACAGAGACUCU | 20 | 4597 |
| CCR5-166 | − | GAUUGUUUAUUUUCUCUUCU | 20 | 4598 |
| CCR5-186 | − | CUUGUGACACGGACUCAAGU | 20 | 4599 |
| CCR5-170 | − | UUGCAGUAGCUCUAACAGGU | 20 | 4600 |
| CCR5-183 | − | GGGGAGCAGGAAAUAUCUGU | 20 | 4601 |
| CCR5-692 | + | CAGGAGAAGGACAAUGUUGU | 20 | 4602 |
| CCR5-174 | − | CCCAUCAUCUAUGCCUUUGU | 20 | 4603 |
| CCR5-160 | − | GAAUCCUAAAAACUCUGCUU | 20 | 4604 |
| CCR5-154 | − | ACAUUAAAGAUAGUCAUCUU | 20 | 4605 |
| CCR5-173 | − | UGCAGGUGACAGAGACUCUU | 20 | 4606 |
| CCR5-168 | − | AACACCUUCCAGGAAUUCUU | 20 | 4607 |
| CCR5-679 | + | ACAGCAUUUGCAGAAGCGUU | 20 | 4608 |
| CCR5-680 | + | AGCGUUUGGCAAUGUGCUUU | 20 | 4609 |
| CCR5-695 | + | GACACCGAAGCAGAGUUUUU | 20 | 4610 |

Table 3A provides exemplary targeting domains for knocking out the CCR5 gene selected according to the first tier parameters. The targeting domains bind within the first 500 bp of the coding sequence (e.g., within 500 bp downstream from the start codon), have a high level of orthogonality and PAM is NNGRRT. It is contemplated herein that in an embodiment the targeting domain hybridizes to the target domain through complementary base pairing. Any of the targeting domains in the table can be used with a S. aureus Cas9 molecule that generates a double stranded break (Cas9 nuclease) or a single-stranded break (Cas9 nickase).

TABLE 3A
1st Tier

| gRNA | DNA | | Target Site | SEQ ID |
|---|---|---|---|---|
| Name | Strand | Targeting Domain | Length | NO |
| CCR5-1878 | + | AUAAAAUAGAGCCCUGUC | 18 | 4611 |
| CCR5-1879 | + | UAUAAAAUAGAGCCCUGUC | 19 | 4612 |
| CCR5-862 | + | CUAUAAAAUAGAGCCCUGUC | 20 | 4613 |
| CCR5-1880 | + | CCUAUAAAAUAGAGCCCUGUC | 21 | 4614 |
| CCR5-1881 | + | GCCUAUAAAAUAGAGCCCUGUC | 22 | 4615 |
| CCR5-1882 | + | AGCCUAUAAAAUAGAGCCCUGUC | 23 | 4616 |
| CCR5-1883 | + | AAGCCUAUAAAAUAGAGCCCUGUC | 24 | 4617 |
| CCR5-1884 | + | UUUGCAGUUUAUCAGGAU | 18 | 4618 |
| CCR5-1885 | + | UUUUGCAGUUUAUCAGGAU | 19 | 4619 |
| CCR5-876 | + | CUUUUGCAGUUUAUCAGGAU | 20 | 4620 |
| CCR5-1886 | − | GGUGACAAGUGUGAUCAC | 18 | 4621 |
| CCR5-1887 | − | UGGUGACAAGUGUGAUCAC | 19 | 4622 |
| CCR5-829 | − | GUGGUGACAAGUGUGAUCAC | 20 | 4623 |
| CCR5-1888 | − | GGUGGUGACAAGUGUGAUCAC | 21 | 4624 |
| CCR5-1889 | − | GGGUGGUGACAAGUGUGAUCAC | 22 | 4625 |
| CCR5-1890 | − | GGGGUGGUGACAAGUGUGAUCAC | 23 | 4626 |
| CCR5-1891 | − | UGGGGUGGUGACAAGUGUGAUCAC | 24 | 4627 |
| CCR5-1892 | − | UUAUGCACAGGGUGGAACAAG | 21 | 4628 |
| CCR5-1893 | − | UUUAUGCACAGGGUGGAACAAG | 22 | 4629 |
| CCR5-1894 | − | AUUUAUGCACAGGGUGGAACAAG | 23 | 4630 |
| CCR5-1895 | − | UAUUUAUGCACAGGGUGGAACAAG | 24 | 4631 |

Table 3B provides exemplary targeting domains for knocking out the CCR5 gene selected according to the second tier parameters. The targeting domains bind within the first 500 bp of the coding sequence (e.g., with 500 bp downstream from the start codon) and PAM is NNGRRT. It is contemplated herein that in an embodiment the targeting domain hybridizes to the target domain through complementary base pairing. Any of the targeting domains in the table can be used with a S. aureus Cas9 molecule that generates a double stranded break (Cas9 nuclease) or a single-stranded break (Cas9 nickase).

TABLE 3B
2nd Tier

| gRNA | DNA | | Target Site | SEQ ID |
|---|---|---|---|---|
| Name | Strand | Targeting Domain | Length | NO |
| CCR5-1896 | + | AACCAAAGAUGAACACCA | 18 | 4632 |
| CCR5-1897 | + | AAACCAAAGAUGAACACCA | 19 | 4633 |
| CCR5-878 | + | AAAACCAAAGAUGAACACCA | 20 | 4634 |
| CCR5-1898 | + | CAAAACCAAAGAUGAACACCA | 21 | 4635 |
| CCR5-1899 | + | ACAAAACCAAAGAUGAACACCA | 22 | 4636 |
| CCR5-1900 | + | CACAAAACCAAAGAUGAACACCA | 23 | 4637 |
| CCR5-1901 | + | CCACAAAACCAAAGAUGAACACCA | 24 | 4638 |
| CCR5-1902 | + | GUACCUAUCGAUUGUCAG | 18 | 4639 |
| CCR5-1903 | + | GGUACCUAUCGAUUGUCAG | 19 | 4640 |
| CCR5-855 | + | AGGUACCUAUCGAUUGUCAG | 20 | 4641 |
| CCR5-1904 | + | CAGGUACCUAUCGAUUGUCAG | 21 | 4642 |
| CCR5-1905 | + | CCAGGUACCUAUCGAUUGUCAG | 22 | 4643 |
| CCR5-1906 | + | GCCAGGUACCUAUCGAUUGUCAG | 23 | 4644 |
| CCR5-1907 | + | AGCCAGGUACCUAUCGAUUGUCAG | 24 | 4645 |
| CCR5-1908 | + | CCUUUUGCAGUUUAUCAGGAU | 21 | 4646 |
| CCR5-1909 | + | GCCUUUUGCAGUUUAUCAGGAU | 22 | 4647 |
| CCR5-1910 | + | AGCCUUUUGCAGUUUAUCAGGAU | 23 | 4648 |
| CCR5-1911 | + | CAGCCUUUUGCAGUUUAUCAGGAU | 24 | 4649 |
| CCR5-1912 | + | CAGCCUUUUGCAGUUUAU | 18 | 4650 |
| CCR5-1913 | + | UCAGCCUUUUGCAGUUUAU | 19 | 4651 |
| CCR5-874 | + | UUCAGCCUUUUGCAGUUUAU | 20 | 4652 |
| CCR5-1914 | + | CUUCAGCCUUUUGCAGUUUAU | 21 | 4653 |
| CCR5-1915 | + | UCUUCAGCCUUUUGCAGUUUAU | 22 | 4654 |
| CCR5-1916 | + | CUCUUCAGCCUUUUGCAGUUUAU | 23 | 4655 |
| CCR5-1917 | + | GCUCUUCAGCCUUUUGCAGUUUAU | 24 | 4656 |
| CCR5-1918 | − | UGUGUUUGCGUCUCUCCC | 18 | 4657 |
| CCR5-1919 | − | CUGUGUUUGCGUCUCUCCC | 19 | 4658 |
| CCR5-59 | − | GCUGUGUUUGCGUCUCUCCC | 20 | 4659 |
| CCR5-1920 | − | GGCUGUGUUUGCGUCUCUCCC | 21 | 4660 |
| CCR5-1921 | − | UGGCUGUGUUUGCGUCUCUCCC | 22 | 4661 |
| CCR5-1922 | − | GUGGCUGUGUUUGCGUCUCUCCC | 23 | 4662 |
| CCR5-1923 | − | GGUGGCUGUGUUUGCGUCUCUCCC | 24 | 4663 |
| CCR5-1924 | − | UUUUAUAGGCUUCUUCUC | 18 | 4664 |
| CCR5-1925 | − | AUUUUAUAGGCUUCUUCUC | 19 | 4665 |
| CCR5-77 | − | UAUUUUAUAGGCUUCUUCUC | 20 | 4666 |
| CCR5-1926 | − | CUAUUUUAUAGGCUUCUUCUC | 21 | 4667 |
| CCR5-1927 | − | UCUAUUUUAUAGGCUUCUUCUC | 22 | 4668 |
| CCR5-1928 | − | CUCUAUUUUAUAGGCUUCUUCUC | 23 | 4669 |
| CCR5-1929 | − | GCUCUAUUUUAUAGGCUUCUUCUC | 24 | 4670 |
| CCR5-1930 | − | UGCACAGGGUGGAACAAG | 18 | 4671 |
| CCR5-1931 | − | AUGCACAGGGUGGAACAAG | 19 | 4672 |
| CCR5-1932 | − | UAUGCACAGGGUGGAACAAG | 20 | 4673 |
| CCR5-1933 | − | AGCCAGGACGGUCACCUU | 18 | 4674 |
| CCR5-1934 | − | AAGCCAGGACGGUCACCUU | 19 | 4675 |
| CCR5-80 | − | AAAGCCAGGACGGUCACCUU | 20 | 4676 |
| CCR5-1935 | − | AAAAGCCAGGACGGUCACCUU | 21 | 4677 |
| CCR5-1936 | − | UAAAAGCCAGGACGGUCACCUU | 22 | 4678 |
| CCR5-1937 | − | UUAAAAGCCAGGACGGUCACCUU | 23 | 4679 |
| CCR5-1938 | − | UUUAAAAGCCAGGACGGUCACCUU | 24 | 4680 |

Table 3C provides exemplary targeting domains for knocking out the CCR5 gene selected according to the third tier parameters. The targeting domains bind within the first 500 bp of the coding sequence (e.g., with 500 bp downstream from the start codon) and PAM is NNGRRV. It is contemplated herein that in an embodiment the targeting domain hybridizes to the target domain through complementary base pairing. Any of the targeting domains in the table can be used with a S. aureus Cas9 molecule that generates a double stranded break (Cas9 nuclease) or a single-stranded break (Cas9 nickase).

TABLE 3C
3rd Tier

| gRNA | DNA | | Target Site | SEQ ID |
|---|---|---|---|---|
| Name | Strand | Targeting Domain | Length | NO |
| CCR5-2255 | + | GAUAUUUCCUGCUCCCCA | 18 | 4681 |
| CCR5-2256 | + | AGAUAUUUCCUGCUCCCCA | 19 | 4682 |
| CCR5-1611 | + | CAGAUAUUUCCUGCUCCCCA | 20 | 4683 |
| CCR5-2257 | + | ACAGAUAUUUCCUGCUCCCCA | 21 | 4684 |
| CCR5-2258 | + | CACAGAUAUUUCCUGCUCCCCA | 22 | 4685 |
| CCR5-2259 | + | CCACAGAUAUUUCCUGCUCCCCA | 23 | 4686 |
| CCR5-2260 | + | CCCACAGAUAUUUCCUGCUCCCCA | 24 | 4687 |
| CCR5-2261 | + | CUGCAAUUAUUCAGGCCA | 18 | 4688 |
| CCR5-2262 | + | ACUGCAAUUAUUCAGGCCA | 19 | 4689 |
| CCR5-1630 | + | UACUGCAAUUAUUCAGGCCA | 20 | 4690 |
| CCR5-2263 | + | CUACUGCAAUUAUUCAGGCCA | 21 | 4691 |
| CCR5-2264 | + | GCUACUGCAAUUAUUCAGGCCA | 22 | 4692 |
| CCR5-2265 | + | AGCUACUGCAAUUAUUCAGGCCA | 23 | 4693 |
| CCR5-2266 | + | GAGCUACUGCAAUUAUUCAGGCCA | 24 | 4694 |
| CCR5-2267 | + | UUCCUGCUCCCCAGUGGA | 18 | 4695 |
| CCR5-2268 | + | UUUCCUGCUCCCCAGUGGA | 19 | 4696 |
| CCR5-1612 | + | AUUUCCUGCUCCCCAGUGGA | 20 | 4697 |
| CCR5-2269 | + | UAUUUCCUGCUCCCCAGUGGA | 21 | 4698 |
| CCR5-2270 | + | AUAUUUCCUGCUCCCCAGUGGA | 22 | 4699 |
| CCR5-2271 | + | GAUAUUUCCUGCUCCCCAGUGGA | 23 | 4700 |
| CCR5-2272 | + | AGAUAUUUCCUGCUCCCCAGUGGA | 24 | 4701 |
| CCR5-2273 | + | CGACAAAGGCAUAGAUGA | 18 | 4702 |
| CCR5-2274 | + | CCGACAAAGGCAUAGAUGA | 19 | 4703 |
| CCR5-683 | + | CCCGACAAAGGCAUAGAUGA | 20 | 4704 |
| CCR5-2275 | + | CCCCGACAAAGGCAUAGAUGA | 21 | 4705 |
| CCR5-2276 | + | UCCCCGACAAAGGCAUAGAUGA | 22 | 4706 |
| CCR5-2277 | + | CUCCCCGACAAAGGCAUAGAUGA | 23 | 4707 |
| CCR5-2278 | + | UCUCCCCGACAAAGGCAUAGAUGA | 24 | 4708 |
| CCR5-2279 | + | GCAGCAGUGCGUCAUCCC | 18 | 4709 |
| CCR5-2280 | + | UGCAGCAGUGCGUCAUCCC | 19 | 4710 |
| CCR5-1628 | + | AUGCAGCAGUGCGUCAUCCC | 20 | 4711 |
| CCR5-2281 | + | GAUGCAGCAGUGCGUCAUCCC | 21 | 4712 |
| CCR5-2282 | + | UGAUGCAGCAGUGCGUCAUCCC | 22 | 4713 |
| CCR5-2283 | + | UUGAUGCAGCAGUGCGUCAUCCC | 23 | 4714 |
| CCR5-2284 | + | GUUGAUGCAGCAGUGCGUCAUCCC | 24 | 4715 |
| CCR5-2285 | + | GCAGAGUUUUUAGGAUUC | 18 | 4716 |
| CCR5-2286 | + | AGCAGAGUUUUUAGGAUUC | 19 | 4717 |
| CCR5-1647 | + | AAGCAGAGUUUUUAGGAUUC | 20 | 4718 |
| CCR5-2287 | + | GAAGCAGAGUUUUUAGGAUUC | 21 | 4719 |
| CCR5-2288 | + | CGAAGCAGAGUUUUUAGGAUUC | 22 | 4720 |
| CCR5-2289 | + | CCGAAGCAGAGUUUUUAGGAUUC | 23 | 4721 |
| CCR5-2290 | + | ACCGAAGCAGAGUUUUUAGGAUUC | 24 | 4722 |
| CCR5-2291 | + | AAUGUCUGGAAAUUCUUC | 18 | 4723 |
| CCR5-2292 | + | UAAUGUCUGGAAAUUCUUC | 19 | 4724 |
| CCR5-1651 | + | UUAAUGUCUGGAAAUUCUUC | 20 | 4725 |
| CCR5-2293 | + | UUUAAUGUCUGGAAAUUCUUC | 21 | 4726 |
| CCR5-2294 | + | CUUUAAUGUCUGGAAAUUCUUC | 22 | 4727 |
| CCR5-2295 | + | UCUUUAAUGUCUGGAAAUUCUUC | 23 | 4728 |
| CCR5-2296 | + | AUCUUUAAUGUCUGGAAAUUCUUC | 24 | 4729 |
| CCR5-2297 | + | CUCAUUUCGACACCGAAG | 18 | 4730 |
| CCR5-2298 | + | UCUCAUUUCGACACCGAAG | 19 | 4731 |
| CCR5-1645 | + | UUCUCAUUUCGACACCGAAG | 20 | 4732 |
| CCR5-2299 | + | CUUCUCAUUUCGACACCGAAG | 21 | 4733 |
| CCR5-2300 | + | UCUUCUCAUUUCGACACCGAAG | 22 | 4734 |
| CCR5-2301 | + | UUCUUCUCAUUUCGACACCGAAG | 23 | 4735 |
| CCR5-2302 | + | CUUCUUCUCAUUUCGACACCGAAG | 24 | 4736 |
| CCR5-2303 | + | ACACCGAAGCAGAGUUUU | 18 | 4737 |
| CCR5-2304 | + | GACACCGAAGCAGAGUUUU | 19 | 4738 |
| CCR5-1646 | + | CGACACCGAAGCAGAGUUUU | 20 | 4739 |
| CCR5-2305 | + | UCGACACCGAAGCAGAGUUUU | 21 | 4740 |
| CCR5-2306 | + | UUCGACACCGAAGCAGAGUUUU | 22 | 4741 |
| CCR5-2307 | + | UUUCGACACCGAAGCAGAGUUUU | 23 | 4742 |
| CCR5-2308 | + | AUUUCGACACCGAAGCAGAGUUUU | 24 | 4743 |
| CCR5-2309 | − | UUCUCCUGAACACCUUCC | 18 | 4744 |
| CCR5-2310 | − | CUUCUCCUGAACACCUUCC | 19 | 4745 |
| CCR5-167 | − | CCUUCUCCUGAACACCUUCC | 20 | 4746 |
| CCR5-2311 | − | UCCUUCUCCUGAACACCUUCC | 21 | 4747 |
| CCR5-2312 | − | GUCCUUCUCCUGAACACCUUCC | 22 | 4748 |
| CCR5-2313 | − | UGUCCUUCUCCUGAACACCUUCC | 23 | 4749 |
| CCR5-2314 | − | UUGUCCUUCUCCUGAACACCUUCC | 24 | 4750 |
| CCR5-2315 | − | UUCCAGGAAUUCUUUGGC | 18 | 4751 |
| CCR5-2316 | − | CUUCCAGGAAUUCUUUGGC | 19 | 4752 |
| CCR5-941 | − | CCUUCCAGGAAUUCUUUGGC | 20 | 4753 |
| CCR5-2317 | − | ACCUUCCAGGAAUUCUUUGGC | 21 | 4754 |
| CCR5-2318 | − | CACCUUCCAGGAAUUCUUUGGC | 22 | 4755 |
| CCR5-2319 | − | ACACCUUCCAGGAAUUCUUUGGC | 23 | 4756 |
| CCR5-2320 | − | AACACCUUCCAGGAAUUCUUUGGC | 24 | 4757 |
| CCR5-2321 | − | CAUGGUCAUCUGCUACUC | 18 | 4758 |
| CCR5-2322 | − | UCAUGGUCAUCUGCUACUC | 19 | 4759 |
| CCR5-159 | − | GUCAUGGUCAUCUGCUACUC | 20 | 4760 |
| CCR5-2323 | − | UGUCAUGGUCAUCUGCUACUC | 21 | 4761 |
| CCR5-2324 | − | UUGUCAUGGUCAUCUGCUACUC | 22 | 4762 |
| CCR5-2325 | − | CUUGUCAUGGUCAUCUGCUACUC | 23 | 4763 |
| CCR5-2326 | − | GCUUGUCAUGGUCAUCUGCUACUC | 24 | 4764 |
| CCR5-2327 | − | AGUCAGUAUCAAUUCUGG | 18 | 4765 |
| CCR5-2328 | − | CAGUCAGUAUCAAUUCUGG | 19 | 4766 |
| CCR5-924 | − | ACAGUCAGUAUCAAUUCUGG | 20 | 4767 |
| CCR5-2329 | − | UACAGUCAGUAUCAAUUCUGG | 21 | 4768 |
| CCR5-2330 | − | AUACAGUCAGUAUCAAUUCUGG | 22 | 4769 |
| CCR5-2331 | − | CAUACAGUCAGUAUCAAUUCUGG | 23 | 4770 |
| CCR5-2332 | − | CCAUACAGUCAGUAUCAAUUCUGG | 24 | 4771 |
| CCR5-2333 | − | GCAGGUGACAGAGACUCU | 18 | 4772 |
| CCR5-2334 | − | UGCAGGUGACAGAGACUCU | 19 | 4773 |
| CCR5-172 | − | AUGCAGGUGACAGAGACUCU | 20 | 4774 |
| CCR5-2335 | − | UAUGCAGGUGACAGAGACUCU | 21 | 4775 |
| CCR5-2336 | − | CUAUGCAGGUGACAGAGACUCU | 22 | 4776 |
| CCR5-2337 | − | GCUAUGCAGGUGACAGAGACUCU | 23 | 4777 |
| CCR5-2338 | − | AGCUAUGCAGGUGACAGAGACUCU | 24 | 4778 |

Table 3D provides exemplary targeting domains for knocking out the CCR5 gene selected according to the fourth tier parameters. The targeting domains fall in the coding sequence of the gene, downstream of the first 500 bp of coding sequence (e.g., anywhere from +500 (relative to the start codon) to the stop codon of the gene.) and PAM is NNGRRT. It is contemplated herein that in an embodiment the targeting domain hybridizes to the target domain through complementary base pairing. Any of the targeting domains in the table can be used with a S. aureus Cas9 molecule that generates a double stranded break (Cas9 nuclease) or a single-stranded break (Cas9 nickase).

TABLE 3D
3rd Tier

| gRNA | DNA | | Target Site | SEQ ID |
|---|---|---|---|---|
| Name | Strand | Targeting Domain | Length | NO |
| CCR5-1939 | + | GAGAAGAAGCCUAUAAAA | 18 | 4779 |
| CCR5-1940 | + | AGAGAAGAAGCCUAUAAAA | 19 | 4780 |
| CCR5-861 | + | CAGAGAAGAAGCCUAUAAAA | 20 | 4781 |
| CCR5-1941 | + | CCAGAGAAGAAGCCUAUAAAA | 21 | 4782 |
| CCR5-1942 | + | UCCAGAGAAGAAGCCUAUAAAA | 22 | 4783 |
| CCR5-1943 | + | UUCCAGAGAAGAAGCCUAUAAAA | 23 | 4784 |
| CCR5-1944 | + | AUUCCAGAGAAGAAGCCUAUAAAA | 24 | 4785 |
| CCR5-1945 | + | AGCAUAGUGAGCCCAGAA | 18 | 4786 |
| CCR5-1946 | + | CAGCAUAGUGAGCCCAGAA | 19 | 4787 |
| CCR5-47 | + | GCAGCAUAGUGAGCCCAGAA | 20 | 4788 |
| CCR5-1947 | + | GGCAGCAUAGUGAGCCCAGAA | 21 | 4789 |
| CCR5-1948 | + | CGGCAGCAUAGUGAGCCCAGAA | 22 | 4790 |
| CCR5-1949 | + | GCGGCAGCAUAGUGAGCCCAGAA | 23 | 4791 |
| CCR5-1950 | + | GGCGGCAGCAUAGUGAGCCCAGAA | 24 | 4792 |
| CCR5-1951 | + | UGUAUUUCCAAAGUCCCA | 18 | 4793 |
| CCR5-1952 | + | UUGUAUUUCCAAAGUCCCA | 19 | 4794 |
| CCR5-863 | + | AUUGUAUUUCCAAAGUCCCA | 20 | 4795 |
| CCR5-1953 | + | CAUUGUAUUUCCAAAGUCCCA | 21 | 4796 |
| CCR5-1954 | + | ACAUUGUAUUUCCAAAGUCCCA | 22 | 4797 |
| CCR5-1955 | + | CACAUUGUAUUUCCAAAGUCCCA | 23 | 4798 |
| CCR5-1956 | + | ACACAUUGUAUUUCCAAAGUCCCA | 24 | 4799 |
| CCR5-1957 | + | AUGAUGAAGAAGAUUCCA | 18 | 4800 |
| CCR5-1958 | + | GAUGAUGAAGAAGAUUCCA | 19 | 4801 |
| CCR5-859 | + | GGAUGAUGAAGAAGAUUCCA | 20 | 4802 |
| CCR5-1959 | + | AGGAUGAUGAAGAAGAUUCCA | 21 | 4803 |
| CCR5-1960 | + | GAGGAUGAUGAAGAAGAUUCCA | 22 | 4804 |
| CCR5-1961 | + | GGAGGAUGAUGAAGAAGAUUCCA | 23 | 4805 |
| CCR5-1962 | + | AGGAGGAUGAUGAAGAAGAUUCCA | 24 | 4806 |
| CCR5-1963 | + | CAGAAGGGGACAGUAAGA | 18 | 4807 |
| CCR5-1964 | + | CCAGAAGGGGACAGUAAGA | 19 | 4808 |
| CCR5-99 | + | CCCAGAAGGGGACAGUAAGA | 20 | 4809 |
| CCR5-1965 | + | GCCCAGAAGGGGACAGUAAGA | 21 | 4810 |
| CCR5-1966 | + | AGCCCAGAAGGGGACAGUAAGA | 22 | 4811 |
| CCR5-1967 | + | GAGCCCAGAAGGGGACAGUAAGA | 23 | 4812 |
| CCR5-1968 | + | UGAGCCCAGAAGGGGACAGUAAGA | 24 | 4813 |
| CCR5-1969 | + | CAGCAUAGUGAGCCCAGA | 18 | 4814 |
| CCR5-1970 | + | GCAGCAUAGUGAGCCCAGA | 19 | 4815 |
| CCR5-46 | + | GGCAGCAUAGUGAGCCCAGA | 20 | 4816 |
| CCR5-1971 | + | CGGCAGCAUAGUGAGCCCAGA | 21 | 4817 |
| CCR5-1972 | + | GCGGCAGCAUAGUGAGCCCAGA | 22 | 4818 |
| CCR5-1973 | + | GGCGGCAGCAUAGUGAGCCCAGA | 23 | 4819 |
| CCR5-1974 | + | GGGCGGCAGCAUAGUGAGCCCAGA | 24 | 4820 |
| CCR5-1975 | + | AAUAAUUGAUGUCAUAGA | 18 | 4821 |
| CCR5-1976 | + | UAAUAAUUGAUGUCAUAGA | 19 | 4822 |
| CCR5-886 | + | AUAAUAAUUGAUGUCAUAGA | 20 | 4823 |
| CCR5-1977 | + | UAUAAUAAUUGAUGUCAUAGA | 21 | 4824 |
| CCR5-1978 | + | GUAUAAUAAUUGAUGUCAUAGA | 22 | 4825 |
| CCR5-1979 | + | UGUAUAAUAAUUGAUGUCAUAGA | 23 | 4826 |
| CCR5-1980 | + | AUGUAUAAUAAUUGAUGUCAUAGA | 24 | 4827 |
| CCR5-1981 | + | UGAACACCAGUGAGUAGA | 18 | 4828 |
| CCR5-1982 | + | AUGAACACCAGUGAGUAGA | 19 | 4829 |
| CCR5-880 | + | GAUGAACACCAGUGAGUAGA | 20 | 4830 |
| CCR5-1983 | + | AGAUGAACACCAGUGAGUAGA | 21 | 4831 |
| CCR5-1984 | + | AAGAUGAACACCAGUGAGUAGA | 22 | 4832 |
| CCR5-1985 | + | AAAGAUGAACACCAGUGAGUAGA | 23 | 4833 |
| CCR5-1986 | + | CAAAGAUGAACACCAGUGAGUAGA | 24 | 4834 |
| CCR5-1987 | + | CCACUGGGCGGCAGCAUA | 18 | 4835 |
| CCR5-1988 | + | CCCACUGGGCGGCAGCAUA | 19 | 4836 |
| CCR5-864 | + | UCCCACUGGGCGGCAGCAUA | 20 | 4837 |
| CCR5-1989 | + | GUCCCACUGGGCGGCAGCAUA | 21 | 4838 |
| CCR5-1990 | + | AGUCCCACUGGGCGGCAGCAUA | 22 | 4839 |
| CCR5-1991 | + | AAGUCCCACUGGGCGGCAGCAUA | 23 | 4840 |
| CCR5-1992 | + | AAAGUCCCACUGGGCGGCAGCAUA | 24 | 4841 |
| CCR5-1993 | + | GCGGCAGCAUAGUGAGCC | 18 | 4842 |
| CCR5-1994 | + | GGCGGCAGCAUAGUGAGCC | 19 | 4843 |
| CCR5-865 | + | GGGCGGCAGCAUAGUGAGCC | 20 | 4844 |
| CCR5-1995 | + | UGGGCGGCAGCAUAGUGAGCC | 21 | 4845 |
| CCR5-1996 | + | CUGGGCGGCAGCAUAGUGAGCC | 22 | 4846 |
| CCR5-1997 | + | ACUGGGCGGCAGCAUAGUGAGCC | 23 | 4847 |
| CCR5-1998 | + | CACUGGGCGGCAGCAUAGUGAGCC | 24 | 4848 |
| CCR5-1999 | + | UCUGGUAAAGAUGAUUCC | 18 | 4849 |
| CCR5-2000 | + | AUCUGGUAAAGAUGAUUCC | 19 | 4850 |
| CCR5-1874 | + | GAUCUGGUAAAGAUGAUUCC | 20 | 4851 |
| CCR5-2001 | + | AGAUCUGGUAAAGAUGAUUCC | 21 | 4852 |
| CCR5-2002 | + | GAGAUCUGGUAAAGAUGAUUCC | 22 | 4853 |
| CCR5-2003 | + | UGAGAUCUGGUAAAGAUGAUUCC | 23 | 4854 |
| CCR5-2004 | + | UUGAGAUCUGGUAAAGAUGAUUCC | 24 | 4855 |
| CCR5-2005 | + | UUUUAAAGCAAACACAGC | 18 | 4856 |
| CCR5-2006 | + | CUUUUAAAGCAAACACAGC | 19 | 4857 |
| CCR5-852 | + | GCUUUUAAAGCAAACACAGC | 20 | 4858 |
| CCR5-2007 | + | GGCUUUUAAAGCAAACACAGC | 21 | 4859 |
| CCR5-2008 | + | UGGCUUUUAAAGCAAACACAGC | 22 | 4860 |
| CCR5-2009 | + | CUGGCUUUUAAAGCAAACACAGC | 23 | 4861 |
| CCR5-2010 | + | CCUGGCUUUUAAAGCAAACACAGC | 24 | 4862 |
| CCR5-2011 | + | AGUGAGUAGAGCGGAGGC | 18 | 4863 |
| CCR5-2012 | + | CAGUGAGUAGAGCGGAGGC | 19 | 4864 |
| CCR5-98 | + | CCAGUGAGUAGAGCGGAGGC | 20 | 4865 |
| CCR5-2013 | + | ACCAGUGAGUAGAGCGGAGGC | 21 | 4866 |
| CCR5-2014 | + | CACCAGUGAGUAGAGCGGAGGC | 22 | 4867 |
| CCR5-2015 | + | ACACCAGUGAGUAGAGCGGAGGC | 23 | 4868 |
| CCR5-2016 | + | AACACCAGUGAGUAGAGCGGAGGC | 24 | 4869 |
| CCR5-2017 | + | AGGUACCUAUCGAUUGUC | 18 | 4870 |
| CCR5-2018 | + | CAGGUACCUAUCGAUUGUC | 19 | 4871 |
| CCR5-60 | + | CCAGGUACCUAUCGAUUGUC | 20 | 4872 |
| CCR5-2019 | + | GCCAGGUACCUAUCGAUUGUC | 21 | 4873 |
| CCR5-2020 | + | AGCCAGGUACCUAUCGAUUGUC | 22 | 4874 |
| CCR5-2021 | + | CAGCCAGGUACCUAUCGAUUGUC | 23 | 4875 |
| CCR5-2022 | + | ACAGCCAGGUACCUAUCGAUUGUC | 24 | 4876 |
| CCR5-2023 | + | GGAUGAUGAAGAAGAUUC | 18 | 4877 |
| CCR5-2024 | + | AGGAUGAUGAAGAAGAUUC | 19 | 4878 |
| CCR5-858 | + | GAGGAUGAUGAAGAAGAUUC | 20 | 4879 |
| CCR5-2025 | + | GGAGGAUGAUGAAGAAGAUUC | 21 | 4880 |
| CCR5-2026 | + | AGGAGGAUGAUGAAGAAGAUUC | 22 | 4881 |
| CCR5-2027 | + | CAGGAGGAUGAUGAAGAAGAUUC | 23 | 4882 |
| CCR5-2028 | + | UCAGGAGGAUGAUGAAGAAGAUUC | 24 | 4883 |
| CCR5-2029 | + | AUCUGGUAAAGAUGAUUC | 18 | 4884 |
| CCR5-2030 | + | GAUCUGGUAAAGAUGAUUC | 19 | 4885 |
| CCR5-2031 | + | AGAUCUGGUAAAGAUGAUUC | 20 | 4886 |
| CCR5-2032 | + | GAGAUCUGGUAAAGAUGAUUC | 21 | 4887 |
| CCR5-2033 | + | UGAGAUCUGGUAAAGAUGAUUC | 22 | 4888 |
| CCR5-2034 | + | UUGAGAUCUGGUAAAGAUGAUUC | 23 | 4889 |
| CCR5-2035 | + | UUUGAGAUCUGGUAAAGAUGAUUC | 24 | 4890 |
| CCR5-2036 | + | UUGCCCACAAAACCAAAG | 18 | 4891 |
| CCR5-2037 | + | GUUGCCCACAAAACCAAAG | 19 | 4892 |
| CCR5-877 | + | UGUUGCCCACAAAACCAAAG | 20 | 4893 |
| CCR5-2038 | + | AUGUUGCCCACAAAACCAAAG | 21 | 4894 |
| CCR5-2039 | + | CAUGUUGCCCACAAAACCAAAG | 22 | 4895 |
| CCR5-2040 | + | GCAUGUUGCCCACAAAACCAAAG | 23 | 4896 |
| CCR5-2041 | + | AGCAUGUUGCCCACAAAACCAAAG | 24 | 4897 |
| CCR5-2042 | + | CCAGAAGGGGACAGUAAG | 18 | 4898 |
| CCR5-2043 | + | CCCAGAAGGGGACAGUAAG | 19 | 4899 |
| CCR5-870 | + | GCCCAGAAGGGGACAGUAAG | 20 | 4900 |
| CCR5-2044 | + | AGCCCAGAAGGGGACAGUAAG | 21 | 4901 |
| CCR5-2045 | + | GAGCCCAGAAGGGGACAGUAAG | 22 | 4902 |
| CCR5-2046 | + | UGAGCCCAGAAGGGGACAGUAAG | 23 | 4903 |
| CCR5-2047 | + | GUGAGCCCAGAAGGGGACAGUAAG | 24 | 4904 |
| CCR5-2048 | + | GCAGCAUAGUGAGCCCAG | 18 | 4905 |
| CCR5-2049 | + | GGCAGCAUAGUGAGCCCAG | 19 | 4906 |
| CCR5-866 | + | CGGCAGCAUAGUGAGCCCAG | 20 | 4907 |
| CCR5-2050 | + | GCGGCAGCAUAGUGAGCCCAG | 21 | 4908 |
| CCR5-2051 | + | GGCGGCAGCAUAGUGAGCCCAG | 22 | 4909 |
| CCR5-2052 | + | GGGCGGCAGCAUAGUGAGCCCAG | 23 | 4910 |
| CCR5-2053 | + | UGGGCGGCAGCAUAGUGAGCCCAG | 24 | 4911 |
| CCR5-2054 | + | AUGAAGAAGAUUCCAGAG | 18 | 4912 |
| CCR5-2055 | + | GAUGAAGAAGAUUCCAGAG | 19 | 4913 |
| CCR5-860 | + | UGAUGAAGAAGAUUCCAGAG | 20 | 4914 |
| CCR5-2056 | + | AUGAUGAAGAAGAUUCCAGAG | 21 | 4915 |
| CCR5-2057 | + | GAUGAUGAAGAAGAUUCCAGAG | 22 | 4916 |
| CCR5-2058 | + | GGAUGAUGAAGAAGAUUCCAGAG | 23 | 4917 |
| CCR5-2059 | + | AGGAUGAUGAAGAAGAUUCCAGAG | 24 | 4918 |
| CCR5-2060 | + | GAACACCAGUGAGUAGAG | 18 | 4919 |
| CCR5-2061 | + | UGAACACCAGUGAGUAGAG | 19 | 4920 |
| CCR5-92 | + | AUGAACACCAGUGAGUAGAG | 20 | 4921 |
| CCR5-2062 | + | GAUGAACACCAGUGAGUAGAG | 21 | 4922 |
| CCR5-2063 | + | AGAUGAACACCAGUGAGUAGAG | 22 | 4923 |
| CCR5-2064 | + | AAGAUGAACACCAGUGAGUAGAG | 23 | 4924 |
| CCR5-2065 | + | AAAGAUGAACACCAGUGAGUAGAG | 24 | 4925 |
| CCR5-2066 | + | GUAGAGCGGAGGCAGGAG | 18 | 4926 |
| CCR5-2067 | + | AGUAGAGCGGAGGCAGGAG | 19 | 4927 |
| CCR5-884 | + | GAGUAGAGCGGAGGCAGGAG | 20 | 4928 |
| CCR5-2068 | + | UGAGUAGAGCGGAGGCAGGAG | 21 | 4929 |
| CCR5-2069 | + | GUGAGUAGAGCGGAGGCAGGAG | 22 | 4930 |
| CCR5-2070 | + | AGUGAGUAGAGCGGAGGCAGGAG | 23 | 4931 |
| CCR5-2071 | + | CAGUGAGUAGAGCGGAGGCAGGAG | 24 | 4932 |
| CCR5-2072 | + | AAGAUGAACACCAGUGAG | 18 | 4933 |
| CCR5-2073 | + | AAAGAUGAACACCAGUGAG | 19 | 4934 |
| CCR5-879 | + | CAAAGAUGAACACCAGUGAG | 20 | 4935 |
| CCR5-2074 | + | CCAAAGAUGAACACCAGUGAG | 21 | 4936 |
| CCR5-2075 | + | ACCAAAGAUGAACACCAGUGAG | 22 | 4937 |
| CCR5-2076 | + | AACCAAAGAUGAACACCAGUGAG | 23 | 4938 |
| CCR5-2077 | + | AAACCAAAGAUGAACACCAGUGAG | 24 | 4939 |
| CCR5-2078 | + | AGGUCAGAGAUGGCCAGG | 18 | 4940 |
| CCR5-2079 | + | CAGGUCAGAGAUGGCCAGG | 19 | 4941 |
| CCR5-873 | + | ACAGGUCAGAGAUGGCCAGG | 20 | 4942 |
| CCR5-2080 | + | AACAGGUCAGAGAUGGCCAGG | 21 | 4943 |
| CCR5-2081 | + | AAACAGGUCAGAGAUGGCCAGG | 22 | 4944 |
| CCR5-2082 | + | AAAACAGGUCAGAGAUGGCCAGG | 23 | 4945 |
| CCR5-2083 | + | AAAAACAGGUCAGAGAUGGCCAGG | 24 | 4946 |
| CCR5-2084 | + | CUUUUGCAGUUUAUCAGG | 18 | 4947 |
| CCR5-2085 | + | CCUUUUGCAGUUUAUCAGG | 19 | 4948 |
| CCR5-875 | + | GCCUUUUGCAGUUUAUCAGG | 20 | 4949 |
| CCR5-2086 | + | AGCCUUUUGCAGUUUAUCAGG | 21 | 4950 |
| CCR5-2087 | + | CAGCCUUUUGCAGUUUAUCAGG | 22 | 4951 |
| CCR5-2088 | + | UCAGCCUUUUGCAGUUUAUCAGG | 23 | 4952 |
| CCR5-2089 | + | UUCAGCCUUUUGCAGUUUAUCAGG | 24 | 4953 |
| CCR5-2090 | + | CAGUGAGUAGAGCGGAGG | 18 | 4954 |
| CCR5-2091 | + | CCAGUGAGUAGAGCGGAGG | 19 | 4955 |
| CCR5-882 | + | ACCAGUGAGUAGAGCGGAGG | 20 | 4956 |
| CCR5-2092 | + | CACCAGUGAGUAGAGCGGAGG | 21 | 4957 |
| CCR5-2093 | + | ACACCAGUGAGUAGAGCGGAGG | 22 | 4958 |
| CCR5-2094 | + | AACACCAGUGAGUAGAGCGGAGG | 23 | 4959 |
| CCR5-2095 | + | GAACACCAGUGAGUAGAGCGGAGG | 24 | 4960 |
| CCR5-2096 | + | GGUAAAGAUGAUUCCUGG | 18 | 4961 |
| CCR5-2097 | + | UGGUAAAGAUGAUUCCUGG | 19 | 4962 |
| CCR5-2098 | + | CUGGUAAAGAUGAUUCCUGG | 20 | 4963 |
| CCR5-2099 | + | UCUGGUAAAGAUGAUUCCUGG | 21 | 4964 |
| CCR5-2100 | + | AUCUGGUAAAGAUGAUUCCUGG | 22 | 4965 |
| CCR5-2101 | + | GAUCUGGUAAAGAUGAUUCCUGG | 23 | 4966 |
| CCR5-2102 | + | AGAUCUGGUAAAGAUGAUUCCUGG | 24 | 4967 |
| CCR5-2103 | + | UUCACAUUGAUUUUUUGG | 18 | 4968 |
| CCR5-2104 | + | CUUCACAUUGAUUUUUUGG | 19 | 4969 |
| CCR5-885 | + | GCUUCACAUUGAUUUUUUGG | 20 | 4970 |
| CCR5-2105 | + | UGCUUCACAUUGAUUUUUUGG | 21 | 4971 |
| CCR5-2106 | + | UUGCUUCACAUUGAUUUUUUGG | 22 | 4972 |
| CCR5-2107 | + | UUUGCUUCACAUUGAUUUUUUGG | 23 | 4973 |
| CCR5-2108 | + | AUUUGCUUCACAUUGAUUUUUUGG | 24 | 4974 |
| CCR5-2109 | + | UCGAUUGUCAGGAGGAUG | 18 | 4975 |
| CCR5-2110 | + | AUCGAUUGUCAGGAGGAUG | 19 | 4976 |
| CCR5-856 | + | UAUCGAUUGUCAGGAGGAUG | 20 | 4977 |
| CCR5-2111 | + | CUAUCGAUUGUCAGGAGGAUG | 21 | 4978 |
| CCR5-2112 | + | CCUAUCGAUUGUCAGGAGGAUG | 22 | 4979 |
| CCR5-2113 | + | ACCUAUCGAUUGUCAGGAGGAUG | 23 | 4980 |
| CCR5-2114 | + | UACCUAUCGAUUGUCAGGAGGAUG | 24 | 4981 |
| CCR5-2115 | + | AUUGUCAGGAGGAUGAUG | 18 | 4982 |
| CCR5-2116 | + | GAUUGUCAGGAGGAUGAUG | 19 | 4983 |
| CCR5-857 | + | CGAUUGUCAGGAGGAUGAUG | 20 | 4984 |
| CCR5-2117 | + | UCGAUUGUCAGGAGGAUGAUG | 21 | 4985 |
| CCR5-2118 | + | AUCGAUUGUCAGGAGGAUGAUG | 22 | 4986 |
| CCR5-2119 | + | UAUCGAUUGUCAGGAGGAUGAUG | 23 | 4987 |
| CCR5-2120 | + | CUAUCGAUUGUCAGGAGGAUGAUG | 24 | 4988 |
| CCR5-2121 | + | CUGGUAAAGAUGAUUCCU | 18 | 4989 |
| CCR5-2122 | + | UCUGGUAAAGAUGAUUCCU | 19 | 4990 |
| CCR5-1877 | + | AUCUGGUAAAGAUGAUUCCU | 20 | 4991 |
| CCR5-2123 | + | GAUCUGGUAAAGAUGAUUCCU | 21 | 4992 |
| CCR5-2124 | + | AGAUCUGGUAAAGAUGAUUCCU | 22 | 4993 |
| CCR5-2125 | + | GAGAUCUGGUAAAGAUGAUUCCU | 23 | 4994 |
| CCR5-2126 | + | UGAGAUCUGGUAAAGAUGAUUCCU | 24 | 4995 |
| CCR5-2127 | + | AGCCCAGAAGGGGACAGU | 18 | 4996 |
| CCR5-2128 | + | GAGCCCAGAAGGGGACAGU | 19 | 4997 |
| CCR5-869 | + | UGAGCCCAGAAGGGGACAGU | 20 | 4998 |
| CCR5-2129 | + | GUGAGCCCAGAAGGGGACAGU | 21 | 4999 |
| CCR5-2130 | + | AGUGAGCCCAGAAGGGGACAGU | 22 | 5000 |
| CCR5-2131 | + | UAGUGAGCCCAGAAGGGGACAGU | 23 | 5001 |
| CCR5-2132 | + | AUAGUGAGCCCAGAAGGGGACAGU | 24 | 5002 |
| CCR5-2133 | + | UAAGAAGGAAAAACAGGU | 18 | 5003 |
| CCR5-2134 | + | GUAAGAAGGAAAAACAGGU | 19 | 5004 |
| CCR5-872 | + | AGUAAGAAGGAAAAACAGGU | 20 | 5005 |
| CCR5-2135 | + | CAGUAAGAAGGAAAAACAGGU | 21 | 5006 |
| CCR5-2136 | + | ACAGUAAGAAGGAAAAACAGGU | 22 | 5007 |
| CCR5-2137 | + | GACAGUAAGAAGGAAAAACAGGU | 23 | 5008 |
| CCR5-2138 | + | GGACAGUAAGAAGGAAAAACAGGU | 24 | 5009 |
| CCR5-2139 | + | CAGGUACCUAUCGAUUGU | 18 | 5010 |
| CCR5-2140 | + | CCAGGUACCUAUCGAUUGU | 19 | 5011 |
| CCR5-853 | + | GCCAGGUACCUAUCGAUUGU | 20 | 5012 |
| CCR5-2141 | + | AGCCAGGUACCUAUCGAUUGU | 21 | 5013 |
| CCR5-2142 | + | CAGCCAGGUACCUAUCGAUUGU | 22 | 5014 |
| CCR5-2143 | + | ACAGCCAGGUACCUAUCGAUUGU | 23 | 5015 |
| CCR5-2144 | + | GACAGCCAGGUACCUAUCGAUUGU | 24 | 5016 |
| CCR5-2145 | + | GUAAUGAAGACCUUCUUU | 18 | 5017 |
| CCR5-2146 | + | UGUAAUGAAGACCUUCUUU | 19 | 5018 |
| CCR5-1657 | + | GUGUAAUGAAGACCUUCUUU | 20 | 5019 |
| CCR5-2147 | − | UCUUUACCAGAUCUCAAA | 18 | 5020 |
| CCR5-2148 | − | AUCUUUACCAGAUCUCAAA | 19 | 5021 |
| CCR5-2149 | − | CAUCUUUACCAGAUCUCAAA | 20 | 5022 |
| CCR5-2150 | − | UCAUCUUUACCAGAUCUCAAA | 21 | 5023 |
| CCR5-2151 | − | AUCAUCUUUACCAGAUCUCAAA | 22 | 5024 |
| CCR5-2152 | − | AAUCAUCUUUACCAGAUCUCAAA | 23 | 5025 |
| CCR5-2153 | − | GAAUCAUCUUUACCAGAUCUCAAA | 24 | 5026 |
| CCR5-2154 | − | GACAUCAAUUAUUAUACA | 18 | 5027 |
| CCR5-2155 | − | UGACAUCAAUUAUUAUACA | 19 | 5028 |
| CCR5-812 | − | AUGACAUCAAUUAUUAUACA | 20 | 5029 |
| CCR5-2156 | − | UAUGACAUCAAUUAUUAUACA | 21 | 5030 |
| CCR5-2157 | − | CUAUGACAUCAAUUAUUAUACA | 22 | 5031 |
| CCR5-2158 | − | UCUAUGACAUCAAUUAUUAUACA | 23 | 5032 |
| CCR5-2159 | − | AUCUAUGACAUCAAUUAUUAUACA | 24 | 5033 |
| CCR5-2160 | − | UCACUAUGCUGCCGCCCA | 18 | 5034 |
| CCR5-2161 | − | CUCACUAUGCUGCCGCCCA | 19 | 5035 |
| CCR5-819 | − | GCUCACUAUGCUGCCGCCCA | 20 | 5036 |
| CCR5-2162 | − | GGCUCACUAUGCUGCCGCCCA | 21 | 5037 |
| CCR5-2163 | − | GGGCUCACUAUGCUGCCGCCCA | 22 | 5038 |
| CCR5-2164 | − | UGGGCUCACUAUGCUGCCGCCCA | 23 | 5039 |
| CCR5-2165 | − | CUGGGCUCACUAUGCUGCCGCCCA | 24 | 5040 |
| CCR5-2166 | − | CAAUGUGUCAACUCUUGA | 18 | 5041 |
| CCR5-2167 | − | ACAAUGUGUCAACUCUUGA | 19 | 5042 |
| CCR5-823 | − | UACAAUGUGUCAACUCUUGA | 20 | 5043 |
| CCR5-2168 | − | AUACAAUGUGUCAACUCUUGA | 21 | 5044 |
| CCR5-2169 | − | AAUACAAUGUGUCAACUCUUGA | 22 | 5045 |
| CCR5-2170 | − | AAAUACAAUGUGUCAACUCUUGA | 23 | 5046 |
| CCR5-2171 | − | GAAAUACAAUGUGUCAACUCUUGA | 24 | 5047 |
| CCR5-2172 | − | CUGUGUUUGCGUCUCUCC | 18 | 5048 |
| CCR5-2173 | − | GCUGUGUUUGCGUCUCUCC | 19 | 5049 |
| CCR5-830 | − | GGCUGUGUUUGCGUCUCUCC | 20 | 5050 |
| CCR5-2174 | − | UGGCUGUGUUUGCGUCUCUCC | 21 | 5051 |
| CCR5-2175 | − | GUGGCUGUGUUUGCGUCUCUCC | 22 | 5052 |
| CCR5-2176 | − | GGUGGCUGUGUUUGCGUCUCUCC | 23 | 5053 |
| CCR5-2177 | − | UGGUGGCUGUGUUUGCGUCUCUCC | 24 | 5054 |
| CCR5-2178 | − | UGUGUUUGCUUUAAAAGC | 18 | 5055 |
| CCR5-2179 | − | CUGUGUUUGCUUUAAAAGC | 19 | 5056 |
| CCR5-826 | − | GCUGUGUUUGCUUUAAAAGC | 20 | 5057 |
| CCR5-2180 | − | UGCUGUGUUUGCUUUAAAAGC | 21 | 5058 |
| CCR5-2181 | − | AUGCUGUGUUUGCUUUAAAAGC | 22 | 5059 |
| CCR5-2182 | − | CAUGCUGUGUUUGCUUUAAAAGC | 23 | 5060 |
| CCR5-2183 | − | CCAUGCUGUGUUUGCUUUAAAAGC | 24 | 5061 |
| CCR5-2184 | − | CACUAUGCUGCCGCCCAG | 18 | 5062 |
| CCR5-2185 | − | UCACUAUGCUGCCGCCCAG | 19 | 5063 |
| CCR5-74 | − | CUCACUAUGCUGCCGCCCAG | 20 | 5064 |
| CCR5-2186 | − | GCUCACUAUGCUGCCGCCCAG | 21 | 5065 |
| CCR5-2187 | − | GGCUCACUAUGCUGCCGCCCAG | 22 | 5066 |
| CCR5-2188 | − | GGGCUCACUAUGCUGCCGCCCAG | 23 | 5067 |
| CCR5-2189 | − | UGGGCUCACUAUGCUGCCGCCCAG | 24 | 5068 |
| CCR5-2190 | − | CUGAUAAACUGCAAAAGG | 18 | 5069 |
| CCR5-2191 | − | CCUGAUAAACUGCAAAAGG | 19 | 5070 |
| CCR5-816 | − | UCCUGAUAAACUGCAAAAGG | 20 | 5071 |
| CCR5-2192 | − | AUCCUGAUAAACUGCAAAAGG | 21 | 5072 |
| CCR5-2193 | − | CAUCCUGAUAAACUGCAAAAGG | 22 | 5073 |
| CCR5-2194 | − | UCAUCCUGAUAAACUGCAAAAGG | 23 | 5074 |
| CCR5-2195 | − | CUCAUCCUGAUAAACUGCAAAAGG | 24 | 5075 |
| CCR5-2196 | − | UUUUUAUUUAUGCACAGG | 18 | 5076 |
| CCR5-2197 | − | CUUUUUAUUUAUGCACAGG | 19 | 5077 |
| CCR5-2198 | − | ACUUUUUAUUUAUGCACAGG | 20 | 5078 |
| CCR5-2199 | − | UUUUAUUUAUGCACAGGG | 18 | 5079 |
| CCR5-2200 | − | UUUUUAUUUAUGCACAGGG | 19 | 5080 |
| CCR5-1876 | − | CUUUUUAUUUAUGCACAGGG | 20 | 5081 |
| CCR5-2201 | − | AUAAACUGCAAAAGGCUG | 18 | 5082 |
| CCR5-2202 | − | GAUAAACUGCAAAAGGCUG | 19 | 5083 |
| CCR5-817 | − | UGAUAAACUGCAAAAGGCUG | 20 | 5084 |
| CCR5-2203 | − | CUGAUAAACUGCAAAAGGCUG | 21 | 5085 |
| CCR5-2204 | − | CCUGAUAAACUGCAAAAGGCUG | 22 | 5086 |
| CCR5-2205 | − | UCCUGAUAAACUGCAAAAGGCUG | 23 | 5087 |
| CCR5-2206 | − | AUCCUGAUAAACUGCAAAAGGCUG | 24 | 5088 |
| CCR5-2207 | − | CCCUGCCAAAAAAUCAAU | 18 | 5089 |
| CCR5-2208 | − | GCCCUGCCAAAAAAUCAAU | 19 | 5090 |
| CCR5-814 | − | AGCCCUGCCAAAAAAUCAAU | 20 | 5091 |
| CCR5-2209 | − | GAGCCCUGCCAAAAAAUCAAU | 21 | 5092 |
| CCR5-2210 | − | GGAGCCCUGCCAAAAAAUCAAU | 22 | 5093 |
| CCR5-2211 | − | CGGAGCCCUGCCAAAAAAUCAAU | 23 | 5094 |
| CCR5-2212 | − | UCGGAGCCCUGCCAAAAAAUCAAU | 24 | 5095 |
| CCR5-2213 | − | ACAUCAAUUAUUAUACAU | 18 | 5096 |
| CCR5-2214 | − | GACAUCAAUUAUUAUACAU | 19 | 5097 |
| CCR5-67 | − | UGACAUCAAUUAUUAUACAU | 20 | 5098 |
| CCR5-2215 | − | AUGACAUCAAUUAUUAUACAU | 21 | 5099 |
| CCR5-2216 | − | UAUGACAUCAAUUAUUAUACAU | 22 | 5100 |
| CCR5-2217 | − | CUAUGACAUCAAUUAUUAUACAU | 23 | 5101 |
| CCR5-2218 | − | UCUAUGACAUCAAUUAUUAUACAU | 24 | 5102 |
| CCR5-2219 | − | CUGCCGCCCAGUGGGACU | 18 | 5103 |
| CCR5-2220 | − | GCUGCCGCCCAGUGGGACU | 19 | 5104 |
| CCR5-821 | − | UGCUGCCGCCCAGUGGGACU | 20 | 5105 |
| CCR5-2221 | − | AUGCUGCCGCCCAGUGGGACU | 21 | 5106 |
| CCR5-2222 | − | UAUGCUGCCGCCCAGUGGGACU | 22 | 5107 |
| CCR5-2223 | − | CUAUGCUGCCGCCCAGUGGGACU | 23 | 5108 |
| CCR5-2224 | − | ACUAUGCUGCCGCCCAGUGGGACU | 24 | 5109 |
| CCR5-2225 | − | AAGCCAGGACGGUCACCU | 18 | 5110 |
| CCR5-2226 | − | AAAGCCAGGACGGUCACCU | 19 | 5111 |
| CCR5-827 | − | AAAAGCCAGGACGGUCACCU | 20 | 5112 |
| CCR5-2227 | − | UAAAAGCCAGGACGGUCACCU | 21 | 5113 |
| CCR5-2228 | − | UUAAAAGCCAGGACGGUCACCU | 22 | 5114 |
| CCR5-2229 | − | UUUAAAAGCCAGGACGGUCACCU | 23 | 5115 |
| CCR5-2230 | − | CUUUAAAAGCCAGGACGGUCACCU | 24 | 5116 |
| CCR5-2231 | − | AUUUUAUAGGCUUCUUCU | 18 | 5117 |
| CCR5-2232 | − | UAUUUUAUAGGCUUCUUCU | 19 | 5118 |
| CCR5-824 | − | CUAUUUUAUAGGCUUCUUCU | 20 | 5119 |
| CCR5-2233 | − | UCUAUUUUAUAGGCUUCUUCU | 21 | 5120 |
| CCR5-2234 | − | CUCUAUUUUAUAGGCUUCUUCU | 22 | 5121 |
| CCR5-2235 | − | GCUCUAUUUUAUAGGCUUCUUCU | 23 | 5122 |
| CCR5-2236 | − | GGCUCUAUUUUAUAGGCUUCUUCU | 24 | 5123 |
| CCR5-2237 | − | UGCCGCCCAGUGGGACUU | 18 | 5124 |
| CCR5-2238 | − | CUGCCGCCCAGUGGGACUU | 19 | 5125 |
| CCR5-43 | − | GCUGCCGCCCAGUGGGACUU | 20 | 5126 |
| CCR5-2239 | − | UGCUGCCGCCCAGUGGGACUU | 21 | 5127 |
| CCR5-2240 | − | AUGCUGCCGCCCAGUGGGACUU | 22 | 5128 |
| CCR5-2241 | − | UAUGCUGCCGCCCAGUGGGACUU | 23 | 5129 |
| CCR5-2242 | − | CUAUGCUGCCGCCCAGUGGGACUU | 24 | 5130 |
| CCR5-2243 | − | CCUUCUUACUGUCCCCUU | 18 | 5131 |
| CCR5-2244 | − | UCCUUCUUACUGUCCCCUU | 19 | 5132 |
| CCR5-818 | − | UUCCUUCUUACUGUCCCCUU | 20 | 5133 |
| CCR5-2245 | − | UUUCCUUCUUACUGUCCCCUU | 21 | 5134 |
| CCR5-2246 | − | UUUUCCUUCUUACUGUCCCCUU | 22 | 5135 |
| CCR5-2247 | − | UUUUUCCUUCUUACUGUCCCCUU | 23 | 5136 |
| CCR5-2248 | − | GUUUUUCCUUCUUACUGUCCCCUU | 24 | 5137 |
| CCR5-2249 | − | GUGUUCAUCUUUGGUUUU | 18 | 5138 |
| CCR5-2250 | − | GGUGUUCAUCUUUGGUUUU | 19 | 5139 |
| CCR5-815 | − | UGGUGUUCAUCUUUGGUUUU | 20 | 5140 |
| CCR5-2251 | − | CUGGUGUUCAUCUUUGGUUUU | 21 | 5141 |
| CCR5-2252 | − | ACUGGUGUUCAUCUUUGGUUUU | 22 | 5142 |
| CCR5-2253 | − | CACUGGUGUUCAUCUUUGGUUUU | 23 | 5143 |
| CCR5-2254 | − | UCACUGGUGUUCAUCUUUGGUUUU | 24 | 5144 |

Table 3E provides exemplary targeting domains for knocking out the CCR5 gene selected according to the fifth tier parameters. The targeting domains fall in the coding sequence of the gene, downstream of the first 500 bp of coding sequence (e.g., anywhere from +500 (relative to the start codon) to the stop codon of the gene and PAM is NNGRRV. It is contemplated herein that in an embodiment the targeting domain hybridizes to the target domain through complementary base pairing. Any of the targeting domains in the table can be used with a S. aureus Cas9 molecule that generates a double stranded break (Cas9 nuclease) or a single-stranded break (Cas9 nickase).

TABLE 3E
5th Tier

| gRNA | DNA | | Target Site | SEQ ID |
|---|---|---|---|---|
| Name | Strand | Targeting Domain | Length | NO |
| CCR5-2339 | + | GAGCCUCUUGCUGGAAAA | 18 | 5145 |
| CCR5-2340 | + | GGAGCCUCUUGCUGGAAAA | 19 | 5146 |
| CCR5-1619 | + | GGGAGCCUCUUGCUGGAAAA | 20 | 5147 |
| CCR5-2341 | + | CGGGAGCCUCUUGCUGGAAAA | 21 | 5148 |
| CCR5-2342 | + | UCGGGAGCCUCUUGCUGGAAAA | 22 | 5149 |
| CCR5-2343 | + | CUCGGGAGCCUCUUGCUGGAAAA | 23 | 5150 |
| CCR5-2344 | + | GCUCGGGAGCCUCUUGCUGGAAAA | 24 | 5151 |
| CCR5-2345 | + | AUACUGACUGUAUGGAAA | 18 | 5152 |
| CCR5-2346 | + | GAUACUGACUGUAUGGAAA | 19 | 5153 |
| CCR5-1654 | + | UGAUACUGACUGUAUGGAAA | 20 | 5154 |
| CCR5-2347 | + | UUGAUACUGACUGUAUGGAAA | 21 | 5155 |
| CCR5-2348 | + | AUUGAUACUGACUGUAUGGAAA | 22 | 5156 |
| CCR5-2349 | + | AAUUGAUACUGACUGUAUGGAAA | 23 | 5157 |
| CCR5-2350 | + | GAAUUGAUACUGACUGUAUGGAAA | 24 | 5158 |
| CCR5-2351 | + | CAGUGGAUCGGGUGUAAA | 18 | 5159 |
| CCR5-2352 | + | CCAGUGGAUCGGGUGUAAA | 19 | 5160 |
| CCR5-1613 | + | CCCAGUGGAUCGGGUGUAAA | 20 | 5161 |
| CCR5-2353 | + | CCCCAGUGGAUCGGGUGUAAA | 21 | 5162 |
| CCR5-2354 | + | UCCCCAGUGGAUCGGGUGUAAA | 22 | 5163 |
| CCR5-2355 | + | CUCCCCAGUGGAUCGGGUGUAAA | 23 | 5164 |
| CCR5-2356 | + | GCUCCCCAGUGGAUCGGGUGUAAA | 24 | 5165 |
| CCR5-2357 | + | GUUGUAGGGAGCCCAGAA | 18 | 5166 |
| CCR5-2358 | + | UGUUGUAGGGAGCCCAGAA | 19 | 5167 |
| CCR5-1642 | + | AUGUUGUAGGGAGCCCAGAA | 20 | 5168 |
| CCR5-2359 | + | AAUGUUGUAGGGAGCCCAGAA | 21 | 5169 |
| CCR5-2360 | + | CAAUGUUGUAGGGAGCCCAGAA | 22 | 5170 |
| CCR5-2361 | + | ACAAUGUUGUAGGGAGCCCAGAA | 23 | 5171 |
| CCR5-2362 | + | GACAAUGUUGUAGGGAGCCCAGAA | 24 | 5172 |
| CCR5-2363 | + | CUUCUUCUCAUUUCGACA | 18 | 5173 |
| CCR5-2364 | + | UCUUCUUCUCAUUUCGACA | 19 | 5174 |
| CCR5-1644 | + | CUCUUCUUCUCAUUUCGACA | 20 | 5175 |
| CCR5-2365 | + | CCUCUUCUUCUCAUUUCGACA | 21 | 5176 |
| CCR5-2366 | + | GCCUCUUCUUCUCAUUUCGACA | 22 | 5177 |
| CCR5-2367 | + | UGCCUCUUCUUCUCAUUUCGACA | 23 | 5178 |
| CCR5-2368 | + | GUGCCUCUUCUUCUCAUUUCGACA | 24 | 5179 |
| CCR5-2369 | + | GAAUUGAUACUGACUGUA | 18 | 5180 |
| CCR5-2370 | + | AGAAUUGAUACUGACUGUA | 19 | 5181 |
| CCR5-699 | + | CAGAAUUGAUACUGACUGUA | 20 | 5182 |
| CCR5-2371 | + | CCAGAAUUGAUACUGACUGUA | 21 | 5183 |
| CCR5-2372 | + | UCCAGAAUUGAUACUGACUGUA | 22 | 5184 |
| CCR5-2373 | + | UUCCAGAAUUGAUACUGACUGUA | 23 | 5185 |
| CCR5-2374 | + | CUUCCAGAAUUGAUACUGACUGUA | 24 | 5186 |
| CCR5-2375 | + | AUGAGAGCUGCAGGUGUA | 18 | 5187 |
| CCR5-2376 | + | AAUGAGAGCUGCAGGUGUA | 19 | 5188 |
| CCR5-1656 | + | AAAUGAGAGCUGCAGGUGUA | 20 | 5189 |
| CCR5-2377 | + | AAAAUGAGAGCUGCAGGUGUA | 21 | 5190 |
| CCR5-2378 | + | GAAAAUGAGAGCUGCAGGUGUA | 22 | 5191 |
| CCR5-2379 | + | GGAAAAUGAGAGCUGCAGGUGUA | 23 | 5192 |
| CCR5-2380 | + | UGGAAAAUGAGAGCUGCAGGUGUA | 24 | 5193 |
| CCR5-2381 | + | GAGAAGGACAAUGUUGUA | 18 | 5194 |
| CCR5-2382 | + | GGAGAAGGACAAUGUUGUA | 19 | 5195 |
| CCR5-693 | + | AGGAGAAGGACAAUGUUGUA | 20 | 5196 |
| CCR5-2383 | + | CAGGAGAAGGACAAUGUUGUA | 21 | 5197 |
| CCR5-2384 | + | UCAGGAGAAGGACAAUGUUGUA | 22 | 5198 |
| CCR5-2385 | + | UUCAGGAGAAGGACAAUGUUGUA | 23 | 5199 |
| CCR5-2386 | + | GUUCAGGAGAAGGACAAUGUUGUA | 24 | 5200 |
| CCR5-2387 | + | ACAAUGUUGUAGGGAGCC | 18 | 5201 |
| CCR5-2388 | + | GACAAUGUUGUAGGGAGCC | 19 | 5202 |
| CCR5-1640 | + | GGACAAUGUUGUAGGGAGCC | 20 | 5203 |
| CCR5-2389 | + | AGGACAAUGUUGUAGGGAGCC | 21 | 5204 |
| CCR5-2390 | + | AAGGACAAUGUUGUAGGGAGCC | 22 | 5205 |
| CCR5-2391 | + | GAAGGACAAUGUUGUAGGGAGCC | 23 | 5206 |
| CCR5-2392 | + | AGAAGGACAAUGUUGUAGGGAGCC | 24 | 5207 |
| CCR5-2393 | + | UUCAGGCCAAAGAAUUCC | 18 | 5208 |
| CCR5-2394 | + | AUUCAGGCCAAAGAAUUCC | 19 | 5209 |
| CCR5-688 | + | UAUUCAGGCCAAAGAAUUCC | 20 | 5210 |
| CCR5-2395 | + | UUAUUCAGGCCAAAGAAUUCC | 21 | 5211 |
| CCR5-2396 | + | AUUAUUCAGGCCAAAGAAUUCC | 22 | 5212 |
| CCR5-2397 | + | AAUUAUUCAGGCCAAAGAAUUCC | 23 | 5213 |
| CCR5-2398 | + | CAAUUAUUCAGGCCAAAGAAUUCC | 24 | 5214 |
| CCR5-1999 | + | UCUGGUAAAGAUGAUUCC | 18 | 5215 |
| CCR5-2000 | + | AUCUGGUAAAGAUGAUUCC | 19 | 5216 |
| CCR5-1874 | + | GAUCUGGUAAAGAUGAUUCC | 20 | 5217 |
| CCR5-2001 | + | AGAUCUGGUAAAGAUGAUUCC | 21 | 5218 |
| CCR5-2002 | + | GAGAUCUGGUAAAGAUGAUUCC | 22 | 5219 |
| CCR5-2003 | + | UGAGAUCUGGUAAAGAUGAUUCC | 23 | 5220 |
| CCR5-2004 | + | UUGAGAUCUGGUAAAGAUGAUUCC | 24 | 5221 |
| CCR5-2399 | + | UAAACUGAGCUUGCUCGC | 18 | 5222 |
| CCR5-2400 | + | GUAAACUGAGCUUGCUCGC | 19 | 5223 |
| CCR5-1614 | + | UGUAAACUGAGCUUGCUCGC | 20 | 5224 |
| CCR5-2401 | + | GUGUAAACUGAGCUUGCUCGC | 21 | 5225 |
| CCR5-2402 | + | GGUGUAAACUGAGCUUGCUCGC | 22 | 5226 |
| CCR5-2403 | + | GGGUGUAAACUGAGCUUGCUCGC | 23 | 5227 |
| CCR5-2404 | + | CGGGUGUAAACUGAGCUUGCUCGC | 24 | 5228 |
| CCR5-2405 | + | CGCUCGGGAGCCUCUUGC | 18 | 5229 |
| CCR5-2406 | + | UCGCUCGGGAGCCUCUUGC | 19 | 5230 |
| CCR5-678 | + | CUCGCUCGGGAGCCUCUUGC | 20 | 5231 |
| CCR5-2407 | + | GCUCGCUCGGGAGCCUCUUGC | 21 | 5232 |
| CCR5-2408 | + | UGCUCGCUCGGGAGCCUCUUGC | 22 | 5233 |
| CCR5-2409 | + | UUGCUCGCUCGGGAGCCUCUUGC | 23 | 5234 |
| CCR5-2410 | + | CUUGCUCGCUCGGGAGCCUCUUGC | 24 | 5235 |
| CCR5-2411 | + | AACUGAGCUUGCUCGCUC | 18 | 5236 |
| CCR5-2412 | + | AAACUGAGCUUGCUCGCUC | 19 | 5237 |
| CCR5-677 | + | UAAACUGAGCUUGCUCGCUC | 20 | 5238 |
| CCR5-2413 | + | GUAAACUGAGCUUGCUCGCUC | 21 | 5239 |
| CCR5-2414 | + | UGUAAACUGAGCUUGCUCGCUC | 22 | 5240 |
| CCR5-2415 | + | GUGUAAACUGAGCUUGCUCGCUC | 23 | 5241 |
| CCR5-2416 | + | GGUGUAAACUGAGCUUGCUCGCUC | 24 | 5242 |
| CCR5-2417 | + | AUGACUAUCUUUAAUGUC | 18 | 5243 |
| CCR5-2418 | + | GAUGACUAUCUUUAAUGUC | 19 | 5244 |
| CCR5-698 | + | AGAUGACUAUCUUUAAUGUC | 20 | 5245 |
| CCR5-2419 | + | AAGAUGACUAUCUUUAAUGUC | 21 | 5246 |
| CCR5-2420 | + | CAAGAUGACUAUCUUUAAUGUC | 22 | 5247 |
| CCR5-2421 | + | CCAAGAUGACUAUCUUUAAUGUC | 23 | 5248 |
| CCR5-2422 | + | CCCAAGAUGACUAUCUUUAAUGUC | 24 | 5249 |
| CCR5-2423 | + | AUUCAGGCCAAAGAAUUC | 18 | 5250 |
| CCR5-2424 | + | UAUUCAGGCCAAAGAAUUC | 19 | 5251 |
| CCR5-1631 | + | UUAUUCAGGCCAAAGAAUUC | 20 | 5252 |
| CCR5-2425 | + | AUUAUUCAGGCCAAAGAAUUC | 21 | 5253 |
| CCR5-2426 | + | AAUUAUUCAGGCCAAAGAAUUC | 22 | 5254 |
| CCR5-2427 | + | CAAUUAUUCAGGCCAAAGAAUUC | 23 | 5255 |
| CCR5-2428 | + | GCAAUUAUUCAGGCCAAAGAAUUC | 24 | 5256 |
| CCR5-2029 | + | AUCUGGUAAAGAUGAUUC | 18 | 5257 |
| CCR5-2030 | + | GAUCUGGUAAAGAUGAUUC | 19 | 5258 |
| CCR5-2031 | + | AGAUCUGGUAAAGAUGAUUC | 20 | 5259 |
| CCR5-2032 | + | GAGAUCUGGUAAAGAUGAUUC | 21 | 5260 |
| CCR5-2033 | + | UGAGAUCUGGUAAAGAUGAUUC | 22 | 5261 |
| CCR5-2034 | + | UUGAGAUCUGGUAAAGAUGAUUC | 23 | 5262 |
| CCR5-2035 | + | UUUGAGAUCUGGUAAAGAUGAUUC | 24 | 5263 |
| CCR5-2429 | + | AAUUCCUGGAAGGUGUUC | 18 | 5264 |
| CCR5-2430 | + | GAAUUCCUGGAAGGUGUUC | 19 | 5265 |
| CCR5-690 | + | AGAAUUCCUGGAAGGUGUUC | 20 | 5266 |
| CCR5-2431 | + | AAGAAUUCCUGGAAGGUGUUC | 21 | 5267 |
| CCR5-2432 | + | AAAGAAUUCCUGGAAGGUGUUC | 22 | 5268 |
| CCR5-2433 | + | CAAAGAAUUCCUGGAAGGUGUUC | 23 | 5269 |
| CCR5-2434 | + | CCAAAGAAUUCCUGGAAGGUGUUC | 24 | 5270 |
| CCR5-2435 | + | AUGUUGUAGGGAGCCCAG | 18 | 5271 |
| CCR5-2436 | + | AAUGUUGUAGGGAGCCCAG | 19 | 5272 |
| CCR5-1641 | + | CAAUGUUGUAGGGAGCCCAG | 20 | 5273 |
| CCR5-2437 | + | ACAAUGUUGUAGGGAGCCCAG | 21 | 5274 |
| CCR5-2438 | + | GACAAUGUUGUAGGGAGCCCAG | 22 | 5275 |
| CCR5-2439 | + | GGACAAUGUUGUAGGGAGCCCAG | 23 | 5276 |
| CCR5-2440 | + | AGGACAAUGUUGUAGGGAGCCCAG | 24 | 5277 |
| CCR5-2441 | + | UUCCUGGAAGGUGUUCAG | 18 | 5278 |
| CCR5-2442 | + | AUUCCUGGAAGGUGUUCAG | 19 | 5279 |
| CCR5-1635 | + | AAUUCCUGGAAGGUGUUCAG | 20 | 5280 |
| CCR5-2443 | + | GAAUUCCUGGAAGGUGUUCAG | 21 | 5281 |
| CCR5-2444 | + | AGAAUUCCUGGAAGGUGUUCAG | 22 | 5282 |
| CCR5-2445 | + | AAGAAUUCCUGGAAGGUGUUCAG | 23 | 5283 |
| CCR5-2446 | + | AAAGAAUUCCUGGAAGGUGUUCAG | 24 | 5284 |
| CCR5-2447 | + | CUGGAAGGUGUUCAGGAG | 18 | 5285 |
| CCR5-2448 | + | CCUGGAAGGUGUUCAGGAG | 19 | 5286 |
| CCR5-1636 | + | UCCUGGAAGGUGUUCAGGAG | 20 | 5287 |
| CCR5-2449 | + | UUCCUGGAAGGUGUUCAGGAG | 21 | 5288 |
| CCR5-2450 | + | AUUCCUGGAAGGUGUUCAGGAG | 22 | 5289 |
| CCR5-2451 | + | AAUUCCUGGAAGGUGUUCAGGAG | 23 | 5290 |
| CCR5-2452 | + | GAAUUCCUGGAAGGUGUUCAGGAG | 24 | 5291 |
| CCR5-2453 | + | GACCAUGACAAGCAGCGG | 18 | 5292 |
| CCR5-2454 | + | UGACCAUGACAAGCAGCGG | 19 | 5293 |
| CCR5-1648 | + | AUGACCAUGACAAGCAGCGG | 20 | 5294 |
| CCR5-2455 | + | GAUGACCAUGACAAGCAGCGG | 21 | 5295 |
| CCR5-2456 | + | AGAUGACCAUGACAAGCAGCGG | 22 | 5296 |
| CCR5-2457 | + | CAGAUGACCAUGACAAGCAGCGG | 23 | 5297 |
| CCR5-2458 | + | GCAGAUGACCAUGACAAGCAGCGG | 24 | 5298 |
| CCR5-2096 | + | GGUAAAGAUGAUUCCUGG | 18 | 5299 |
| CCR5-2097 | + | UGGUAAAGAUGAUUCCUGG | 19 | 5300 |
| CCR5-2098 | + | CUGGUAAAGAUGAUUCCUGG | 20 | 5301 |
| CCR5-2099 | + | UCUGGUAAAGAUGAUUCCUGG | 21 | 5302 |
| CCR5-2100 | + | AUCUGGUAAAGAUGAUUCCUGG | 22 | 5303 |
| CCR5-2101 | + | GAUCUGGUAAAGAUGAUUCCUGG | 23 | 5304 |
| CCR5-2102 | + | AGAUCUGGUAAAGAUGAUUCCUGG | 24 | 5305 |
| CCR5-2459 | + | UUGGCAAUGUGCUUUUGG | 18 | 5306 |
| CCR5-2460 | + | UUUGGCAAUGUGCUUUUGG | 19 | 5307 |
| CCR5-1623 | + | GUUUGGCAAUGUGCUUUUGG | 20 | 5308 |
| CCR5-2461 | + | CGUUUGGCAAUGUGCUUUUGG | 21 | 5309 |
| CCR5-2462 | + | GCGUUUGGCAAUGUGCUUUUGG | 22 | 5310 |
| CCR5-2463 | + | AGCGUUUGGCAAUGUGCUUUUGG | 23 | 5311 |
| CCR5-2464 | + | AAGCGUUUGGCAAUGUGCUUUUGG | 24 | 5312 |
| CCR5-2465 | + | CCGACAAAGGCAUAGAUG | 18 | 5313 |
| CCR5-2466 | + | CCCGACAAAGGCAUAGAUG | 19 | 5314 |
| CCR5-1626 | + | CCCCGACAAAGGCAUAGAUG | 20 | 5315 |
| CCR5-2467 | + | UCCCCGACAAAGGCAUAGAUG | 21 | 5316 |
| CCR5-2468 | + | CUCCCCGACAAAGGCAUAGAUG | 22 | 5317 |
| CCR5-2469 | + | UCUCCCCGACAAAGGCAUAGAUG | 23 | 5318 |
| CCR5-2470 | + | UUCUCCCCGACAAAGGCAUAGAUG | 24 | 5319 |
| CCR5-2471 | + | AAAUAAACAAUCAUGAUG | 18 | 5320 |
| CCR5-2472 | + | AAAAUAAACAAUCAUGAUG | 19 | 5321 |
| CCR5-1643 | + | GAAAAUAAACAAUCAUGAUG | 20 | 5322 |
| CCR5-2473 | + | AGAAAAUAAACAAUCAUGAUG | 21 | 5323 |
| CCR5-2474 | + | GAGAAAAUAAACAAUCAUGAUG | 22 | 5324 |
| CCR5-2475 | + | AGAGAAAAUAAACAAUCAUGAUG | 23 | 5325 |
| CCR5-2476 | + | AAGAGAAAAUAAACAAUCAUGAUG | 24 | 5326 |
| CCR5-2477 | + | UCGCUCGGGAGCCUCUUG | 18 | 5327 |
| CCR5-2478 | + | CUCGCUCGGGAGCCUCUUG | 19 | 5328 |
| CCR5-1617 | + | GCUCGCUCGGGAGCCUCUUG | 20 | 5329 |
| CCR5-2479 | + | UGCUCGCUCGGGAGCCUCUUG | 21 | 5330 |
| CCR5-2480 | + | UUGCUCGCUCGGGAGCCUCUUG | 22 | 5331 |
| CCR5-2481 | + | CUUGCUCGCUCGGGAGCCUCUUG | 23 | 5332 |
| CCR5-2482 | + | GCUUGCUCGCUCGGGAGCCUCUUG | 24 | 5333 |
| CCR5-2483 | + | AGGAGAAGGACAAUGUUG | 18 | 5334 |
| CCR5-2484 | + | CAGGAGAAGGACAAUGUUG | 19 | 5335 |
| CCR5-1637 | + | UCAGGAGAAGGACAAUGUUG | 20 | 5336 |
| CCR5-2485 | + | UUCAGGAGAAGGACAAUGUUG | 21 | 5337 |
| CCR5-2486 | + | GUUCAGGAGAAGGACAAUGUUG | 22 | 5338 |
| CCR5-2487 | + | UGUUCAGGAGAAGGACAAUGUUG | 23 | 5339 |
| CCR5-2488 | + | GUGUUCAGGAGAAGGACAAUGUUG | 24 | 5340 |
| CCR5-2489 | + | AAAAUAGAACAGCAUUUG | 18 | 5341 |
| CCR5-2490 | + | GAAAAUAGAACAGCAUUUG | 19 | 5342 |
| CCR5-1620 | + | GGAAAAUAGAACAGCAUUUG | 20 | 5343 |
| CCR5-2491 | + | UGGAAAAUAGAACAGCAUUUG | 21 | 5344 |
| CCR5-2492 | + | CUGGAAAAUAGAACAGCAUUUG | 22 | 5345 |
| CCR5-2493 | + | GCUGGAAAAUAGAACAGCAUUUG | 23 | 5346 |
| CCR5-2494 | + | UGCUGGAAAAUAGAACAGCAUUUG | 24 | 5347 |
| CCR5-2495 | + | ACUGACUGUAUGGAAAAU | 18 | 5348 |
| CCR5-2496 | + | UACUGACUGUAUGGAAAAU | 19 | 5349 |
| CCR5-1655 | + | AUACUGACUGUAUGGAAAAU | 20 | 5350 |
| CCR5-2497 | + | GAUACUGACUGUAUGGAAAAU | 21 | 5351 |
| CCR5-2498 | + | UGAUACUGACUGUAUGGAAAAU | 22 | 5352 |
| CCR5-2499 | + | UUGAUACUGACUGUAUGGAAAAU | 23 | 5353 |
| CCR5-2500 | + | AUUGAUACUGACUGUAUGGAAAAU | 24 | 5354 |
| CCR5-2501 | + | UGCUUUUGGAAGAAGACU | 18 | 5355 |
| CCR5-2502 | + | GUGCUUUUGGAAGAAGACU | 19 | 5356 |
| CCR5-1624 | + | UGUGCUUUUGGAAGAAGACU | 20 | 5357 |
| CCR5-2503 | + | AUGUGCUUUUGGAAGAAGACU | 21 | 5358 |
| CCR5-2504 | + | AAUGUGCUUUUGGAAGAAGACU | 22 | 5359 |
| CCR5-2505 | + | CAAUGUGCUUUUGGAAGAAGACU | 23 | 5360 |
| CCR5-2506 | + | GCAAUGUGCUUUUGGAAGAAGACU | 24 | 5361 |
| CCR5-2121 | + | CUGGUAAAGAUGAUUCCU | 18 | 5362 |
| CCR5-2122 | + | UCUGGUAAAGAUGAUUCCU | 19 | 5363 |
| CCR5-1877 | + | AUCUGGUAAAGAUGAUUCCU | 20 | 5364 |
| CCR5-2123 | + | GAUCUGGUAAAGAUGAUUCCU | 21 | 5365 |
| CCR5-2124 | + | AGAUCUGGUAAAGAUGAUUCCU | 22 | 5366 |
| CCR5-2125 | + | GAGAUCUGGUAAAGAUGAUUCCU | 23 | 5367 |
| CCR5-2126 | + | UGAGAUCUGGUAAAGAUGAUUCCU | 24 | 5368 |
| CCR5-2507 | + | AAACUGAGCUUGCUCGCU | 18 | 5369 |
| CCR5-2508 | + | UAAACUGAGCUUGCUCGCU | 19 | 5370 |
| CCR5-676 | + | GUAAACUGAGCUUGCUCGCU | 20 | 5371 |
| CCR5-2509 | + | UGUAAACUGAGCUUGCUCGCU | 21 | 5372 |
| CCR5-2510 | + | GUGUAAACUGAGCUUGCUCGCU | 22 | 5373 |
| CCR5-2511 | + | GGUGUAAACUGAGCUUGCUCGCU | 23 | 5374 |
| CCR5-2512 | + | GGGUGUAAACUGAGCUUGCUCGCU | 24 | 5375 |
| CCR5-2513 | + | GAUGACUAUCUUUAAUGU | 18 | 5376 |
| CCR5-2514 | + | AGAUGACUAUCUUUAAUGU | 19 | 5377 |
| CCR5-1649 | + | AAGAUGACUAUCUUUAAUGU | 20 | 5378 |
| CCR5-2515 | + | CAAGAUGACUAUCUUUAAUGU | 21 | 5379 |
| CCR5-2516 | + | CCAAGAUGACUAUCUUUAAUGU | 22 | 5380 |
| CCR5-2517 | + | CCCAAGAUGACUAUCUUUAAUGU | 23 | 5381 |
| CCR5-2518 | + | CCCCAAGAUGACUAUCUUUAAUGU | 24 | 5382 |
| CCR5-2519 | + | AGAAUUGAUACUGACUGU | 18 | 5383 |
| CCR5-2520 | + | CAGAAUUGAUACUGACUGU | 19 | 5384 |
| CCR5-1652 | + | CCAGAAUUGAUACUGACUGU | 20 | 5385 |
| CCR5-2521 | + | UCCAGAAUUGAUACUGACUGU | 21 | 5386 |
| CCR5-2522 | + | UUCCAGAAUUGAUACUGACUGU | 22 | 5387 |
| CCR5-2523 | + | CUUCCAGAAUUGAUACUGACUGU | 23 | 5388 |
| CCR5-2524 | + | UCUUCCAGAAUUGAUACUGACUGU | 24 | 5389 |
| CCR5-2525 | + | UAGCUUGGUCCAACCUGU | 18 | 5390 |
| CCR5-2526 | + | AUAGCUUGGUCCAACCUGU | 19 | 5391 |
| CCR5-1629 | + | CAUAGCUUGGUCCAACCUGU | 20 | 5392 |
| CCR5-2527 | + | GCAUAGCUUGGUCCAACCUGU | 21 | 5393 |
| CCR5-2528 | + | UGCAUAGCUUGGUCCAACCUGU | 22 | 5394 |
| CCR5-2529 | + | CUGCAUAGCUUGGUCCAACCUGU | 23 | 5395 |
| CCR5-2530 | + | CCUGCAUAGCUUGGUCCAACCUGU | 24 | 5396 |
| CCR5-2531 | + | GGAGAAGGACAAUGUUGU | 18 | 5397 |
| CCR5-2532 | + | AGGAGAAGGACAAUGUUGU | 19 | 5398 |
| CCR5-692 | + | CAGGAGAAGGACAAUGUUGU | 20 | 5399 |
| CCR5-2533 | + | UCAGGAGAAGGACAAUGUUGU | 21 | 5400 |
| CCR5-2534 | + | UUCAGGAGAAGGACAAUGUUGU | 22 | 5401 |
| CCR5-2535 | + | GUUCAGGAGAAGGACAAUGUUGU | 23 | 5402 |
| CCR5-2536 | + | UGUUCAGGAGAAGGACAAUGUUGU | 24 | 5403 |
| CCR5-2537 | + | GCGUUUGGCAAUGUGCUU | 18 | 5404 |
| CCR5-2538 | + | AGCGUUUGGCAAUGUGCUU | 19 | 5405 |
| CCR5-1621 | + | AAGCGUUUGGCAAUGUGCUU | 20 | 5406 |
| CCR5-2539 | + | GAAGCGUUUGGCAAUGUGCUU | 21 | 5407 |
| CCR5-2540 | + | AGAAGCGUUUGGCAAUGUGCUU | 22 | 5408 |
| CCR5-2541 | + | CAGAAGCGUUUGGCAAUGUGCUU | 23 | 5409 |
| CCR5-2542 | + | GCAGAAGCGUUUGGCAAUGUGCUU | 24 | 5410 |
| CCR5-2543 | + | GAAUUCCUGGAAGGUGUU | 18 | 5411 |
| CCR5-2544 | + | AGAAUUCCUGGAAGGUGUU | 19 | 5412 |
| CCR5-1633 | + | AAGAAUUCCUGGAAGGUGUU | 20 | 5413 |
| CCR5-2545 | + | AAAGAAUUCCUGGAAGGUGUU | 21 | 5414 |
| CCR5-2546 | + | CAAAGAAUUCCUGGAAGGUGUU | 22 | 5415 |
| CCR5-2547 | + | CCAAAGAAUUCCUGGAAGGUGUU | 23 | 5416 |
| CCR5-2548 | + | GCCAAAGAAUUCCUGGAAGGUGUU | 24 | 5417 |
| CCR5-2549 | + | CGUUUGGCAAUGUGCUUU | 18 | 5418 |
| CCR5-2550 | + | GCGUUUGGCAAUGUGCUUU | 19 | 5419 |
| CCR5-680 | + | AGCGUUUGGCAAUGUGCUUU | 20 | 5420 |
| CCR5-2551 | + | AAGCGUUUGGCAAUGUGCUUU | 21 | 5421 |
| CCR5-2552 | + | GAAGCGUUUGGCAAUGUGCUUU | 22 | 5422 |
| CCR5-2553 | + | AGAAGCGUUUGGCAAUGUGCUUU | 23 | 5423 |
| CCR5-2554 | + | CAGAAGCGUUUGGCAAUGUGCUUU | 24 | 5424 |
| CCR5-2145 | + | GUAAUGAAGACCUUCUUU | 18 | 5425 |
| CCR5-2146 | + | UGUAAUGAAGACCUUCUUU | 19 | 5426 |
| CCR5-1657 | + | GUGUAAUGAAGACCUUCUUU | 20 | 5427 |
| CCR5-2555 | + | GGUGUAAUGAAGACCUUCUUU | 21 | 5428 |
| CCR5-2556 | + | AGGUGUAAUGAAGACCUUCUUU | 22 | 5429 |
| CCR5-2557 | + | CAGGUGUAAUGAAGACCUUCUUU | 23 | 5430 |
| CCR5-2558 | + | GCAGGUGUAAUGAAGACCUUCUUU | 24 | 5431 |
| CCR5-2559 | + | AAGACUAAGAGGUAGUUU | 18 | 5432 |
| CCR5-2560 | + | GAAGACUAAGAGGUAGUUU | 19 | 5433 |
| CCR5-1625 | + | AGAAGACUAAGAGGUAGUUU | 20 | 5434 |
| CCR5-2561 | + | AAGAAGACUAAGAGGUAGUUU | 21 | 5435 |
| CCR5-2562 | + | GAAGAAGACUAAGAGGUAGUUU | 22 | 5436 |
| CCR5-2563 | + | GGAAGAAGACUAAGAGGUAGUUU | 23 | 5437 |
| CCR5-2564 | + | UGGAAGAAGACUAAGAGGUAGUUU | 24 | 5438 |
| CCR5-2147 | − | UCUUUACCAGAUCUCAAA | 18 | 5439 |
| CCR5-2148 | − | AUCUUUACCAGAUCUCAAA | 19 | 5440 |
| CCR5-2149 | − | CAUCUUUACCAGAUCUCAAA | 20 | 5441 |
| CCR5-2150 | − | UCAUCUUUACCAGAUCUCAAA | 21 | 5442 |
| CCR5-2151 | − | AUCAUCUUUACCAGAUCUCAAA | 22 | 5443 |
| CCR5-2152 | − | AAUCAUCUUUACCAGAUCUCAAA | 23 | 5444 |
| CCR5-2153 | − | GAAUCAUCUUUACCAGAUCUCAAA | 24 | 5445 |
| CCR5-2565 | − | CUUGUGACACGGACUCAA | 18 | 5446 |
| CCR5-2566 | − | GCUUGUGACACGGACUCAA | 19 | 5447 |
| CCR5-963 | − | GGCUUGUGACACGGACUCAA | 20 | 5448 |
| CCR5-2567 | − | GGGCUUGUGACACGGACUCAA | 21 | 5449 |
| CCR5-2568 | − | UGGGCUUGUGACACGGACUCAA | 22 | 5450 |
| CCR5-2569 | − | GUGGGCUUGUGACACGGACUCAA | 23 | 5451 |
| CCR5-2570 | − | UGUGGGCUUGUGACACGGACUCAA | 24 | 5452 |
| CCR5-2571 | − | CUCUGCUUCGGUGUCGAA | 18 | 5453 |
| CCR5-2572 | − | ACUCUGCUUCGGUGUCGAA | 19 | 5454 |
| CCR5-931 | − | AACUCUGCUUCGGUGUCGAA | 20 | 5455 |
| CCR5-2573 | − | AAACUCUGCUUCGGUGUCGAA | 21 | 5456 |
| CCR5-2574 | − | AAAACUCUGCUUCGGUGUCGAA | 22 | 5457 |
| CCR5-2575 | − | AAAAACUCUGCUUCGGUGUCGAA | 23 | 5458 |
| CCR5-2576 | − | UAAAAACUCUGCUUCGGUGUCGAA | 24 | 5459 |
| CCR5-2577 | − | CAGUUUACACCCGAUCCA | 18 | 5460 |
| CCR5-2578 | − | UCAGUUUACACCCGAUCCA | 19 | 5461 |
| CCR5-955 | − | CUCAGUUUACACCCGAUCCA | 20 | 5462 |
| CCR5-2579 | − | GCUCAGUUUACACCCGAUCCA | 21 | 5463 |
| CCR5-2580 | − | AGCUCAGUUUACACCCGAUCCA | 22 | 5464 |
| CCR5-2581 | − | AAGCUCAGUUUACACCCGAUCCA | 23 | 5465 |
| CCR5-2582 | − | CAAGCUCAGUUUACACCCGAUCCA | 24 | 5466 |
| CCR5-2583 | − | AAAUGAGAAGAAGAGGCA | 18 | 5467 |
| CCR5-2584 | − | GAAAUGAGAAGAAGAGGCA | 19 | 5468 |
| CCR5-935 | − | CGAAAUGAGAAGAAGAGGCA | 20 | 5469 |
| CCR5-2585 | − | UCGAAAUGAGAAGAAGAGGCA | 21 | 5470 |
| CCR5-2586 | − | GUCGAAAUGAGAAGAAGAGGCA | 22 | 5471 |
| CCR5-2587 | − | UGUCGAAAUGAGAAGAAGAGGCA | 23 | 5472 |
| CCR5-2588 | − | GUGUCGAAAUGAGAAGAAGAGGCA | 24 | 5473 |
| CCR5-2589 | − | CCAGCAAGAGGCUCCCGA | 18 | 5474 |
| CCR5-2590 | − | UCCAGCAAGAGGCUCCCGA | 19 | 5475 |
| CCR5-954 | − | UUCCAGCAAGAGGCUCCCGA | 20 | 5476 |
| CCR5-2591 | − | UUUCCAGCAAGAGGCUCCCGA | 21 | 5477 |
| CCR5-2592 | − | UUUUCCAGCAAGAGGCUCCCGA | 22 | 5478 |
| CCR5-2593 | − | AUUUUCCAGCAAGAGGCUCCCGA | 23 | 5479 |
| CCR5-2594 | − | UAUUUUCCAGCAAGAGGCUCCCGA | 24 | 5480 |
| CCR5-2595 | − | ACCAAGCUAUGCAGGUGA | 18 | 5481 |
| CCR5-2596 | − | GACCAAGCUAUGCAGGUGA | 19 | 5482 |
| CCR5-943 | − | GGACCAAGCUAUGCAGGUGA | 20 | 5483 |
| CCR5-2597 | − | UGGACCAAGCUAUGCAGGUGA | 21 | 5484 |
| CCR5-2598 | − | UUGGACCAAGCUAUGCAGGUGA | 22 | 5485 |
| CCR5-2599 | − | GUUGGACCAAGCUAUGCAGGUGA | 23 | 5486 |
| CCR5-2600 | − | GGUUGGACCAAGCUAUGCAGGUGA | 24 | 5487 |
| CCR5-2601 | − | AGUUUACACCCGAUCCAC | 18 | 5488 |
| CCR5-2602 | − | CAGUUUACACCCGAUCCAC | 19 | 5489 |
| CCR5-178 | − | UCAGUUUACACCCGAUCCAC | 20 | 5490 |
| CCR5-2603 | − | CUCAGUUUACACCCGAUCCAC | 21 | 5491 |
| CCR5-2604 | − | GCUCAGUUUACACCCGAUCCAC | 22 | 5492 |
| CCR5-2605 | − | AGCUCAGUUUACACCCGAUCCAC | 23 | 5493 |
| CCR5-2606 | − | AAGCUCAGUUUACACCCGAUCCAC | 24 | 5494 |
| CCR5-2607 | − | UAUCUGUGGGCUUGUGAC | 18 | 5495 |
| CCR5-2608 | − | AUAUCUGUGGGCUUGUGAC | 19 | 5496 |
| CCR5-962 | − | AAUAUCUGUGGGCUUGUGAC | 20 | 5497 |
| CCR5-2609 | − | AAAUAUCUGUGGGCUUGUGAC | 21 | 5498 |
| CCR5-2610 | − | GAAAUAUCUGUGGGCUUGUGAC | 22 | 5499 |
| CCR5-2611 | − | GGAAAUAUCUGUGGGCUUGUGAC | 23 | 5500 |
| CCR5-2612 | − | AGGAAAUAUCUGUGGGCUUGUGAC | 24 | 5501 |
| CCR5-2613 | − | GUCAUGGUCAUCUGCUAC | 18 | 5502 |
| CCR5-2614 | − | UGUCAUGGUCAUCUGCUAC | 19 | 5503 |
| CCR5-927 | − | UUGUCAUGGUCAUCUGCUAC | 20 | 5504 |
| CCR5-2615 | − | CUUGUCAUGGUCAUCUGCUAC | 21 | 5505 |
| CCR5-2616 | − | GCUUGUCAUGGUCAUCUGCUAC | 22 | 5506 |
| CCR5-2617 | − | UGCUUGUCAUGGUCAUCUGCUAC | 23 | 5507 |
| CCR5-2618 | − | CUGCUUGUCAUGGUCAUCUGCUAC | 24 | 5508 |
| CCR5-2619 | − | GCUGUUCUAUUUUCCAGC | 18 | 5509 |
| CCR5-2620 | − | UGCUGUUCUAUUUUCCAGC | 19 | 5510 |
| CCR5-952 | − | AUGCUGUUCUAUUUUCCAGC | 20 | 5511 |
| CCR5-2621 | − | AAUGCUGUUCUAUUUUCCAGC | 21 | 5512 |
| CCR5-2622 | − | AAAUGCUGUUCUAUUUUCCAGC | 22 | 5513 |
| CCR5-2623 | − | CAAAUGCUGUUCUAUUUUCCAGC | 23 | 5514 |
| CCR5-2624 | − | GCAAAUGCUGUUCUAUUUUCCAGC | 24 | 5515 |
| CCR5-2625 | − | CCCGAUCCACUGGGGAGC | 18 | 5516 |
| CCR5-2626 | − | ACCCGAUCCACUGGGGAGC | 19 | 5517 |
| CCR5-181 | − | CACCCGAUCCACUGGGGAGC | 20 | 5518 |
| CCR5-2627 | − | ACACCCGAUCCACUGGGGAGC | 21 | 5519 |
| CCR5-2628 | − | UACACCCGAUCCACUGGGGAGC | 22 | 5520 |
| CCR5-2629 | − | UUACACCCGAUCCACUGGGGAGC | 23 | 5521 |
| CCR5-2630 | − | UUUACACCCGAUCCACUGGGGAGC | 24 | 5522 |
| CCR5-2631 | − | ACAUUAAAGAUAGUCAUC | 18 | 5523 |
| CCR5-2632 | − | GACAUUAAAGAUAGUCAUC | 19 | 5524 |
| CCR5-925 | − | AGACAUUAAAGAUAGUCAUC | 20 | 5525 |
| CCR5-2633 | − | CAGACAUUAAAGAUAGUCAUC | 21 | 5526 |
| CCR5-2634 | − | CCAGACAUUAAAGAUAGUCAUC | 22 | 5527 |
| CCR5-2635 | − | UCCAGACAUUAAAGAUAGUCAUC | 23 | 5528 |
| CCR5-2636 | − | UUCCAGACAUUAAAGAUAGUCAUC | 24 | 5529 |
| CCR5-2637 | − | UGCAGGUGACAGAGACUC | 18 | 5530 |
| CCR5-2638 | − | AUGCAGGUGACAGAGACUC | 19 | 5531 |
| CCR5-944 | − | UAUGCAGGUGACAGAGACUC | 20 | 5532 |
| CCR5-2639 | − | CUAUGCAGGUGACAGAGACUC | 21 | 5533 |
| CCR5-2640 | − | GCUAUGCAGGUGACAGAGACUC | 22 | 5534 |
| CCR5-2641 | − | AGCUAUGCAGGUGACAGAGACUC | 23 | 5535 |
| CCR5-2642 | − | AAGCUAUGCAGGUGACAGAGACUC | 24 | 5536 |
| CCR5-2643 | − | UUUUCCAGCAAGAGGCUC | 18 | 5537 |
| CCR5-2644 | − | AUUUUCCAGCAAGAGGCUC | 19 | 5538 |
| CCR5-953 | − | UAUUUUCCAGCAAGAGGCUC | 20 | 5539 |
| CCR5-2645 | − | CUAUUUUCCAGCAAGAGGCUC | 21 | 5540 |
| CCR5-2646 | − | UCUAUUUUCCAGCAAGAGGCUC | 22 | 5541 |
| CCR5-2647 | − | UUCUAUUUUCCAGCAAGAGGCUC | 23 | 5542 |
| CCR5-2648 | − | GUUCUAUUUUCCAGCAAGAGGCUC | 24 | 5543 |
| CCR5-2649 | − | UACAACAUUGUCCUUCUC | 18 | 5544 |
| CCR5-2650 | − | CUACAACAUUGUCCUUCUC | 19 | 5545 |
| CCR5-938 | − | CCUACAACAUUGUCCUUCUC | 20 | 5546 |
| CCR5-2651 | − | CCCUACAACAUUGUCCUUCUC | 21 | 5547 |
| CCR5-2652 | − | UCCCUACAACAUUGUCCUUCUC | 22 | 5548 |
| CCR5-2653 | − | CUCCCUACAACAUUGUCCUUCUC | 23 | 5549 |
| CCR5-2654 | − | GCUCCCUACAACAUUGUCCUUCUC | 24 | 5550 |
| CCR5-2655 | − | AUCAUCUAUGCCUUUGUC | 18 | 5551 |
| CCR5-2656 | − | CAUCAUCUAUGCCUUUGUC | 19 | 5552 |
| CCR5-175 | − | CCAUCAUCUAUGCCUUUGUC | 20 | 5553 |
| CCR5-2657 | − | CCCAUCAUCUAUGCCUUUGUC | 21 | 5554 |
| CCR5-2658 | − | CCCCAUCAUCUAUGCCUUUGUC | 22 | 5555 |
| CCR5-2659 | − | ACCCCAUCAUCUAUGCCUUUGUC | 23 | 5556 |
| CCR5-2660 | − | AACCCCAUCAUCUAUGCCUUUGUC | 24 | 5557 |
| CCR5-2661 | − | UACAGUCAGUAUCAAUUC | 18 | 5558 |
| CCR5-2662 | − | AUACAGUCAGUAUCAAUUC | 19 | 5559 |
| CCR5-152 | − | CAUACAGUCAGUAUCAAUUC | 20 | 5560 |
| CCR5-2663 | − | CCAUACAGUCAGUAUCAAUUC | 21 | 5561 |
| CCR5-2664 | − | UCCAUACAGUCAGUAUCAAUUC | 22 | 5562 |
| CCR5-2665 | − | UUCCAUACAGUCAGUAUCAAUUC | 23 | 5563 |
| CCR5-2666 | − | UUUCCAUACAGUCAGUAUCAAUUC | 24 | 5564 |
| CCR5-2667 | − | CUUCUCCUGAACACCUUC | 18 | 5565 |
| CCR5-2668 | − | CCUUCUCCUGAACACCUUC | 19 | 5566 |
| CCR5-939 | − | UCCUUCUCCUGAACACCUUC | 20 | 5567 |
| CCR5-2669 | − | GUCCUUCUCCUGAACACCUUC | 21 | 5568 |
| CCR5-2670 | − | UGUCCUUCUCCUGAACACCUUC | 22 | 5569 |
| CCR5-2671 | − | UUGUCCUUCUCCUGAACACCUUC | 23 | 5570 |
| CCR5-2672 | − | AUUGUCCUUCUCCUGAACACCUUC | 24 | 5571 |
| CCR5-2673 | − | CGGUGUCGAAAUGAGAAG | 18 | 5572 |
| CCR5-2674 | − | UCGGUGUCGAAAUGAGAAG | 19 | 5573 |
| CCR5-934 | − | UUCGGUGUCGAAAUGAGAAG | 20 | 5574 |
| CCR5-2675 | − | CUUCGGUGUCGAAAUGAGAAG | 21 | 5575 |
| CCR5-2676 | − | GCUUCGGUGUCGAAAUGAGAAG | 22 | 5576 |
| CCR5-2677 | − | UGCUUCGGUGUCGAAAUGAGAAG | 23 | 5577 |
| CCR5-2678 | − | CUGCUUCGGUGUCGAAAUGAGAAG | 24 | 5578 |
| CCR5-2679 | − | ACCCGAUCCACUGGGGAG | 18 | 5579 |
| CCR5-2680 | − | CACCCGAUCCACUGGGGAG | 19 | 5580 |
| CCR5-959 | − | ACACCCGAUCCACUGGGGAG | 20 | 5581 |
| CCR5-2681 | − | UACACCCGAUCCACUGGGGAG | 21 | 5582 |
| CCR5-2682 | − | UUACACCCGAUCCACUGGGGAG | 22 | 5583 |
| CCR5-2683 | − | UUUACACCCGAUCCACUGGGGAG | 23 | 5584 |
| CCR5-2684 | − | GUUUACACCCGAUCCACUGGGGAG | 24 | 5585 |
| CCR5-2685 | − | CUUCGGUGUCGAAAUGAG | 18 | 5586 |
| CCR5-2686 | − | GCUUCGGUGUCGAAAUGAG | 19 | 5587 |
| CCR5-933 | − | UGCUUCGGUGUCGAAAUGAG | 20 | 5588 |
| CCR5-2687 | − | CUGCUUCGGUGUCGAAAUGAG | 21 | 5589 |
| CCR5-2688 | − | UCUGCUUCGGUGUCGAAAUGAG | 22 | 5590 |
| CCR5-2689 | − | CUCUGCUUCGGUGUCGAAAUGAG | 23 | 5591 |
| CCR5-2690 | − | ACUCUGCUUCGGUGUCGAAAUGAG | 24 | 5592 |
| CCR5-2691 | − | UCAUCUAUGCCUUUGUCG | 18 | 5593 |
| CCR5-2692 | − | AUCAUCUAUGCCUUUGUCG | 19 | 5594 |
| CCR5-176 | − | CAUCAUCUAUGCCUUUGUCG | 20 | 5595 |
| CCR5-2693 | − | CCAUCAUCUAUGCCUUUGUCG | 21 | 5596 |
| CCR5-2694 | − | CCCAUCAUCUAUGCCUUUGUCG | 22 | 5597 |
| CCR5-2695 | − | CCCCAUCAUCUAUGCCUUUGUCG | 23 | 5598 |
| CCR5-2696 | − | ACCCCAUCAUCUAUGCCUUUGUCG | 24 | 5599 |
| CCR5-2697 | − | UGCAGUAGCUCUAACAGG | 18 | 5600 |
| CCR5-2698 | − | UUGCAGUAGCUCUAACAGG | 19 | 5601 |
| CCR5-942 | − | AUUGCAGUAGCUCUAACAGG | 20 | 5602 |
| CCR5-2699 | − | AAUUGCAGUAGCUCUAACAGG | 21 | 5603 |
| CCR5-2700 | − | UAAUUGCAGUAGCUCUAACAGG | 22 | 5604 |
| CCR5-2701 | − | AUAAUUGCAGUAGCUCUAACAGG | 23 | 5605 |
| CCR5-2702 | − | AAUAAUUGCAGUAGCUCUAACAGG | 24 | 5606 |
| CCR5-2703 | − | AUCUAUGCCUUUGUCGGG | 18 | 5607 |
| CCR5-2704 | − | CAUCUAUGCCUUUGUCGGG | 19 | 5608 |
| CCR5-950 | − | UCAUCUAUGCCUUUGUCGGG | 20 | 5609 |
| CCR5-2705 | − | AUCAUCUAUGCCUUUGUCGGG | 21 | 5610 |
| CCR5-2706 | − | CAUCAUCUAUGCCUUUGUCGGG | 22 | 5611 |
| CCR5-2707 | − | CCAUCAUCUAUGCCUUUGUCGGG | 23 | 5612 |
| CCR5-2708 | − | CCCAUCAUCUAUGCCUUUGUCGGG | 24 | 5613 |
| CCR5-2709 | − | UUUACACCCGAUCCACUG | 18 | 5614 |
| CCR5-2710 | − | GUUUACACCCGAUCCACUG | 19 | 5615 |
| CCR5-180 | − | AGUUUACACCCGAUCCACUG | 20 | 5616 |
| CCR5-2711 | − | CAGUUUACACCCGAUCCACUG | 21 | 5617 |
| CCR5-2712 | − | UCAGUUUACACCCGAUCCACUG | 22 | 5618 |
| CCR5-2713 | − | CUCAGUUUACACCCGAUCCACUG | 23 | 5619 |
| CCR5-2714 | − | GCUCAGUUUACACCCGAUCCACUG | 24 | 5620 |
| CCR5-2715 | − | AAAAACUCUGCUUCGGUG | 18 | 5621 |
| CCR5-2716 | − | UAAAAACUCUGCUUCGGUG | 19 | 5622 |
| CCR5-930 | − | CUAAAAACUCUGCUUCGGUG | 20 | 5623 |
| CCR5-2717 | − | CCUAAAAACUCUGCUUCGGUG | 21 | 5624 |
| CCR5-2718 | − | UCCUAAAAACUCUGCUUCGGUG | 22 | 5625 |
| CCR5-2719 | − | AUCCUAAAAACUCUGCUUCGGUG | 23 | 5626 |
| CCR5-2720 | − | AAUCCUAAAAACUCUGCUUCGGUG | 24 | 5627 |
| CCR5-2721 | − | CCAUCAUCUAUGCCUUUG | 18 | 5628 |
| CCR5-2722 | − | CCCAUCAUCUAUGCCUUUG | 19 | 5629 |
| CCR5-946 | − | CCCCAUCAUCUAUGCCUUUG | 20 | 5630 |
| CCR5-2723 | − | ACCCCAUCAUCUAUGCCUUUG | 21 | 5631 |
| CCR5-2724 | − | AACCCCAUCAUCUAUGCCUUUG | 22 | 5632 |
| CCR5-2725 | − | CAACCCCAUCAUCUAUGCCUUUG | 23 | 5633 |
| CCR5-2726 | − | UCAACCCCAUCAUCUAUGCCUUUG | 24 | 5634 |
| CCR5-2727 | − | CUGCUUCGGUGUCGAAAU | 18 | 5635 |
| CCR5-2728 | − | UCUGCUUCGGUGUCGAAAU | 19 | 5636 |
| CCR5-932 | − | CUCUGCUUCGGUGUCGAAAU | 20 | 5637 |
| CCR5-2729 | − | ACUCUGCUUCGGUGUCGAAAU | 21 | 5638 |
| CCR5-2730 | − | AACUCUGCUUCGGUGUCGAAAU | 22 | 5639 |
| CCR5-2731 | − | AAACUCUGCUUCGGUGUCGAAAU | 23 | 5640 |
| CCR5-2732 | − | AAAACUCUGCUUCGGUGUCGAAAU | 24 | 5641 |
| CCR5-2733 | − | GUUUACACCCGAUCCACU | 18 | 5642 |
| CCR5-2734 | − | AGUUUACACCCGAUCCACU | 19 | 5643 |
| CCR5-179 | − | CAGUUUACACCCGAUCCACU | 20 | 5644 |
| CCR5-2735 | − | UCAGUUUACACCCGAUCCACU | 21 | 5645 |
| CCR5-2736 | − | CUCAGUUUACACCCGAUCCACU | 22 | 5646 |
| CCR5-2737 | − | GCUCAGUUUACACCCGAUCCACU | 23 | 5647 |
| CCR5-2738 | − | AGCUCAGUUUACACCCGAUCCACU | 24 | 5648 |
| CCR5-2739 | − | UCAUGGUCAUCUGCUACU | 18 | 5649 |
| CCR5-2740 | − | GUCAUGGUCAUCUGCUACU | 19 | 5650 |
| CCR5-158 | − | UGUCAUGGUCAUCUGCUACU | 20 | 5651 |
| CCR5-2741 | − | UUGUCAUGGUCAUCUGCUACU | 21 | 5652 |
| CCR5-2742 | − | CUUGUCAUGGUCAUCUGCUACU | 22 | 5653 |
| CCR5-2743 | − | GCUUGUCAUGGUCAUCUGCUACU | 23 | 5654 |
| CCR5-2744 | − | UGCUUGUCAUGGUCAUCUGCUACU | 24 | 5655 |
| CCR5-2745 | − | AAGAAGAGGCACAGGGCU | 18 | 5656 |
| CCR5-2746 | − | GAAGAAGAGGCACAGGGCU | 19 | 5657 |
| CCR5-936 | − | AGAAGAAGAGGCACAGGGCU | 20 | 5658 |
| CCR5-2747 | − | GAGAAGAAGAGGCACAGGGCU | 21 | 5659 |
| CCR5-2748 | − | UGAGAAGAAGAGGCACAGGGCU | 22 | 5660 |
| CCR5-2749 | − | AUGAGAAGAAGAGGCACAGGGCU | 23 | 5661 |
| CCR5-2750 | − | AAUGAGAAGAAGAGGCACAGGGCU | 24 | 5662 |
| CCR5-2751 | − | CAUUAAAGAUAGUCAUCU | 18 | 5663 |
| CCR5-2752 | − | ACAUUAAAGAUAGUCAUCU | 19 | 5664 |
| CCR5-153 | − | GACAUUAAAGAUAGUCAUCU | 20 | 5665 |
| CCR5-2753 | − | AGACAUUAAAGAUAGUCAUCU | 21 | 5666 |
| CCR5-2754 | − | CAGACAUUAAAGAUAGUCAUCU | 22 | 5667 |
| CCR5-2755 | − | CCAGACAUUAAAGAUAGUCAUCU | 23 | 5668 |
| CCR5-2756 | − | UCCAGACAUUAAAGAUAGUCAUCU | 24 | 5669 |
| CCR5-2757 | − | GGGGAGCAGGAAAUAUCU | 18 | 5670 |
| CCR5-2758 | − | UGGGGAGCAGGAAAUAUCU | 19 | 5671 |
| CCR5-961 | − | CUGGGGAGCAGGAAAUAUCU | 20 | 5672 |
| CCR5-2759 | − | ACUGGGGAGCAGGAAAUAUCU | 21 | 5673 |
| CCR5-2760 | − | CACUGGGGAGCAGGAAAUAUCU | 22 | 5674 |
| CCR5-2761 | − | CCACUGGGGAGCAGGAAAUAUCU | 23 | 5675 |
| CCR5-2762 | − | UCCACUGGGGAGCAGGAAAUAUCU | 24 | 5676 |
| CCR5-2763 | − | CAUCAUCUAUGCCUUUGU | 18 | 5677 |
| CCR5-2764 | − | CCAUCAUCUAUGCCUUUGU | 19 | 5678 |
| CCR5-174 | − | CCCAUCAUCUAUGCCUUUGU | 20 | 5679 |
| CCR5-2765 | − | CCCCAUCAUCUAUGCCUUUGU | 21 | 5680 |
| CCR5-2766 | − | ACCCCAUCAUCUAUGCCUUUGU | 22 | 5681 |
| CCR5-2767 | − | AACCCCAUCAUCUAUGCCUUUGU | 23 | 5682 |
| CCR5-2768 | − | CAACCCCAUCAUCUAUGCCUUUGU | 24 | 5683 |
| CCR5-2769 | − | AUACAGUCAGUAUCAAUU | 18 | 5684 |
| CCR5-2770 | − | CAUACAGUCAGUAUCAAUU | 19 | 5685 |
| CCR5-922 | − | CCAUACAGUCAGUAUCAAUU | 20 | 5686 |
| CCR5-2771 | − | UCCAUACAGUCAGUAUCAAUU | 21 | 5687 |
| CCR5-2772 | − | UUCCAUACAGUCAGUAUCAAUU | 22 | 5688 |
| CCR5-2773 | − | UUUCCAUACAGUCAGUAUCAAUU | 23 | 5689 |
| CCR5-2774 | − | UUUUCCAUACAGUCAGUAUCAAUU | 24 | 5690 |
| CCR5-2775 | − | GAUUGUUUAUUUUCUCUU | 18 | 5691 |
| CCR5-2776 | − | UGAUUGUUUAUUUUCUCUU | 19 | 5692 |
| CCR5-937 | − | AUGAUUGUUUAUUUUCUCUU | 20 | 5693 |
| CCR5-2777 | − | CAUGAUUGUUUAUUUUCUCUU | 21 | 5694 |
| CCR5-2778 | − | UCAUGAUUGUUUAUUUUCUCUU | 22 | 5695 |
| CCR5-2779 | − | AUCAUGAUUGUUUAUUUUCUCUU | 23 | 5696 |
| CCR5-2780 | − | CAUCAUGAUUGUUUAUUUUCUCUU | 24 | 5697 |
| CCR5-2781 | − | CUUUGUCGGGGAGAAGUU | 18 | 5698 |
| CCR5-2782 | − | CCUUUGUCGGGGAGAAGUU | 19 | 5699 |
| CCR5-951 | − | GCCUUUGUCGGGGAGAAGUU | 20 | 5700 |
| CCR5-2783 | − | UGCCUUUGUCGGGGAGAAGUU | 21 | 5701 |
| CCR5-2784 | − | AUGCCUUUGUCGGGGAGAAGUU | 22 | 5702 |
| CCR5-2785 | − | UAUGCCUUUGUCGGGGAGAAGUU | 23 | 5703 |
| CCR5-2786 | − | CUAUGCCUUUGUCGGGGAGAAGUU | 24 | 5704 |

Table 4A provides exemplary targeting domains for knocking out the CCR5 gene selected according to the first tier parameters. The targeting domains bind within the first 500 bp of the coding sequence (e.g., with 500 bp downstream from the start codon) and have a high level of orthogonality. It is contemplated herein that in an embodiment the targeting domain hybridizes to the target domain through complementary base pairing. Any of the targeting domains in the table can be used with a N. meningitidis Cas9 molecule that generates a double stranded break (Cas9 nuclease) or a single-stranded break (Cas9 nickase).

TABLE 4A
1st Tier

| | | | Target | |
|---|---|---|---|---|
| gRNA | DNA | | Site | SEQ ID |
| Name | Strand | Targeting Domain | Length | NO |
| CCR5-2787 | − | UGCACAGGGUGGAACAA | 17 | 5705 |
| CCR5-1824 | + | GGCUGCGAUUUGCUUCA | 17 | 5706 |
| CCR5-1821 | + | GACGACAGCCAGGUACC | 17 | 5707 |
| CCR5-1823 | + | CGGAGGCAGGAGGCGGG | 17 | 5708 |
| CCR5-1825 | + | UGUAUAAUAAUUGAUGU | 17 | 5709 |
| CCR5-2788 | − | GCUGUCGUCCAUGCUGU | 17 | 5710 |
| CCR5-2789 | − | UGACAGGGCUCUAUUUU | 17 | 5711 |
| CCR5-2790 | − | UUAUGCACAGGGUGGAACAA | 20 | 5712 |
| CCR5-1819 | + | GCGGGCUGCGAUUUGCUUCA | 20 | 5713 |
| CCR5-1816 | + | AUGGACGACAGCCAGGUACC | 20 | 5714 |
| CCR5-1818 | + | GAGCGGAGGCAGGAGGCGGG | 20 | 5715 |
| CCR5-1820 | + | CGAUGUAUAAUAAUUGAUGU | 20 | 5716 |
| CCR5-2791 | − | UCUUGACAGGGCUCUAUUUU | 20 | 5717 |

Table 4B provides exemplary targeting domains for knocking out the CCR5 gene selected according to the second tier parameters. The targeting domains bind within the first 500 bp of the coding sequence (e.g., with 500 bp downstream from the start codon). It is contemplated herein that in an embodiment the targeting domain hybridizes to the target domain through complementary base pairing. Any of the targeting domains in the table can be used with a N. meningitidis Cas9 molecule that generates a double stranded break (Cas9 nuclease) or a single-stranded break (Cas9 nickase).

TABLE 4B
2nd Tier

| | | | Target | |
|---|---|---|---|---|
| gRNA | DNA | | Site | SEQ ID |
| Name | Strand | Targeting Domain | Length | NO |
| CCR5-2792 | + | UUUUUGAGAUCUGGUAA | 17 | 5718 |
| CCR5-1822 | + | UGUCAGGAGGAUGAUGA | 17 | 5719 |
| CCR5-2793 | + | GCAGGAGGCGGGCUGCG | 17 | 5720 |
| CCR5-2794 | + | ACCCCAAAGGUGACCGU | 17 | 5721 |
| CCR5-2795 | + | UUCUUUUUGAGAUCUGGUAA | 20 | 5722 |
| CCR5-1817 | + | GAUUGUCAGGAGGAUGAUGA | 20 | 5723 |
| CCR5-2796 | + | GAGGCAGGAGGCGGGCUGCG | 20 | 5724 |
| CCR5-2797 | + | ACCACCCCAAAGGUGACCGU | 20 | 5725 |
| CCR5-2798 | − | CUGGCUGUCGUCCAUGCUGU | 20 | 5726 |

Table 4C provides exemplary targeting domains for knocking out the CCR5 gene selected according to the third tier parameters. The targeting domains fall in the coding sequence of the gene, downstream of the first 500 bp of coding sequence (e.g., anywhere from +500 (relative to the start codon) to the stop codon of the gene. It is contemplated herein that in an embodiment the targeting domain hybridizes to the target domain through complementary base pairing. Any of the targeting domains in the table can be used with a N. meningitidis Cas9 molecule that generates a double stranded break (Cas9 nuclease) or a single-stranded break (Cas9 nickase).

TABLE 4C
3rd Tier

| | | | Target | |
|---|---|---|---|---|
| gRNA | DNA | | Site | SEQ ID |
| Name | Strand | Targeting Domain | Length | NO |
| CCR5-2799 | − | CUCGGGAAUCCUAAAAA | 17 | 5727 |
| CCR5-1771 | + | AGUGGAUCGGGUGUAAA | 17 | 5728 |
| CCR5-2792 | + | UUUUUGAGAUCUGGUAA | 17 | 5729 |
| CCR5-1841 | − | GAGGCUUAUCUUCACCA | 17 | 5730 |
| CCR5-2800 | + | UGCAGAAGCGUUUGGCA | 17 | 5731 |
| CCR5-2801 | − | UCCAAAAGCACAUUGCC | 17 | 5732 |
| CCR5-2802 | − | CUUGGGGCUGGUCCUGC | 17 | 5733 |
| CCR5-2803 | + | AGAGUCUCUGUCACCUG | 17 | 5734 |
| CCR5-2804 | − | GAAGAGGCACAGGGCUG | 17 | 5735 |
| CCR5-1250 | − | GGGAGCAGGAAAUAUCU | 17 | 5736 |
| CCR5-1863 | + | ACACCGAAGCAGAGUUU | 17 | 5737 |
| CCR5-2805 | − | CUACUCGGGAAUCCUAAAAA | 20 | 5738 |
| CCR5-1613 | + | CCCAGUGGAUCGGGUGUAAA | 20 | 5739 |
| CCR5-2795 | + | UUCUUUUUGAGAUCUGGUAA | 20 | 5740 |
| CCR5-1826 | − | UGUGAGGCUUAUCUUCACCA | 20 | 5741 |
| CCR5-2806 | + | AUUUGCAGAAGCGUUUGGCA | 20 | 5742 |
| CCR5-2807 | − | UCUUCCAAAAGCACAUUGCC | 20 | 5743 |
| CCR5-2808 | − | CAUCUUGGGGCUGGUCCUGC | 20 | 5744 |
| CCR5-2809 | + | CCAAGAGUCUCUGUCACCUG | 20 | 5745 |
| CCR5-2810 | − | GAAGAAGAGGCACAGGGCUG | 20 | 5746 |
| CCR5-961 | − | CUGGGGAGCAGGAAAUAUCU | 20 | 5747 |
| CCR5-1859 | + | UCGACACCGAAGCAGAGUUU | 20 | 5748 |

Table 5A provides exemplary targeting domains for knocking down the CCR5 gene selected according to the first tier parameters. The targeting domains bind within 500 bp (e.g., upstream or downstream) of a transcription start site (TSS) and have a high level of orthogonality. It is contemplated herein that in an embodiment the targeting domain hybridizes to the target domain through complementary base pairing. Any of the targeting domains in the table can be used with a S. pyogenes eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 5A
1st Tier

| | | | Target | |
|---|---|---|---|---|
| gRNA | DNA | | Site | SEQ ID |
| Name | Strand | Targeting Domain | Length | NO |
| CCR5-2811 | + | CUCAGAAGCUAACUAAC | 17 | 2217 |
| CCR5-2812 | + | UUACGGGCUUUUCUCAC | 17 | 2218 |
| CCR5-2813 | + | UGAGAGGUUACUUACCG | 17 | 2219 |
| CCR5-2814 | + | AGAAUAGAUCUCUGGUCUGA | 20 | 2220 |
| CCR5-2815 | + | CUGGUCUGAAGGUUUAUUUA | 20 | 2221 |
| CCR5-2816 | + | CAUCUCAGAAGCUAACUAAC | 20 | 2222 |
| CCR5-2817 | + | UGGUCUGAAGGUUUAUUUAC | 20 | 2223 |
| CCR5-2818 | − | CCCCUACAAGAAACUCUCCC | 20 | 2224 |
| CCR5-2819 | − | GAUAGGGGAUACGGGGAGAG | 20 | 2225 |
| CCR5-2820 | + | CCGGGGAGAGUUUCUUGUAG | 20 | 2226 |
| CCR5-2821 | + | AGCUGAGAGGUUACUUACCG | 20 | 2227 |
| CCR5-2822 | + | AAGAUAAUUGUAUGAGCACU | 20 | 2228 |
| CCR5-2823 | − | UCCCCCUCUACAUUUAAAGU | 20 | 2229 |

Table 5B provides exemplary targeting domains for knocking down the CCR5 gene selected according to the second tier parameters. The targeting domains bind within 500 bp (e.g., upstream or downstream) of a transcription start site (TSS). It is contemplated herein that in an embodiment the targeting domain hybridizes to the target domain through complementary base pairing. Any of the targeting domains in the table can be used with a S. pyogenes eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNA may be used to target an eiCas9 to the promoter region of the CCR5 gene.

TABLE 5B
2nd Tier

| | | | Target | |
|---|---|---|---|---|
| gRNA | DNA | | Site | SEQ ID |
| Name | Strand | Targeting Domain | Length | NO |
| CCR5-2824 | − | GGGAGAGUGGAGAAAAA | 17 | 2230 |
| CCR5-2825 | − | GGGGAGAGUGGAGAAAA | 17 | 2231 |
| CCR5-2826 | − | UCUUUAAGAUAAGGAAA | 17 | 2232 |
| CCR5-2827 | + | UCAACAGUAAGGCUAAA | 17 | 2233 |
| CCR5-2828 | − | GAGUGAAAGACUUUAAA | 17 | 2234 |
| CCR5-2829 | − | AUCUUUAAGAUAAGGAA | 17 | 2235 |
| CCR5-2830 | + | AGUUUCUUGUAGGGGAA | 17 | 2236 |
| CCR5-2831 | + | GAAAAUAUAAAGAAUAA | 17 | 2237 |
| CCR5-2832 | − | UGAGUGAAAGACUUUAA | 17 | 2238 |
| CCR5-2833 | − | GAGAAAAAGGGGACACA | 17 | 2239 |
| CCR5-2834 | + | AUUUGUACAAGAUCACA | 17 | 2240 |
| CCR5-2835 | − | UUGGAAUGAGUUUCAGA | 17 | 2241 |
| CCR5-2836 | + | AGGCAUCUCACUGGAGA | 17 | 2242 |
| CCR5-2837 | + | CCAACUUUAAAUGUAGA | 17 | 2243 |
| CCR5-2838 | + | CUGUUUCUUUUGAAGGA | 17 | 2244 |
| CCR5-2839 | + | AUAGAUCUCUGGUCUGA | 17 | 2245 |
| CCR5-2840 | + | AUCAUUAAGUGUAUUGA | 17 | 2246 |
| CCR5-2841 | + | AAUGCUGUUUCUUUUGA | 17 | 2247 |
| CCR5-2842 | − | AUAUAAUCUUUAAGAUA | 17 | 2248 |
| CCR5-2843 | − | GGGUGGGAUAGGGGAUA | 17 | 2249 |
| CCR5-2844 | − | GGGGUUGGGGUGGGAUA | 17 | 2250 |
| CCR5-2845 | − | AAUCUUAUCUUCUGCUA | 17 | 2251 |
| CCR5-2846 | + | UUGCCAAAUGUCUUCUA | 17 | 2252 |
| CCR5-2847 | + | AGGGCUUUUCAACAGUA | 17 | 2253 |
| CCR5-2848 | + | CUUUCUUUUGAGAGGUA | 17 | 2254 |
| CCR5-2849 | + | GGGGAGAGUUUCUUGUA | 17 | 2255 |
| CCR5-2850 | + | GUCUGAAGGUUUAUUUA | 17 | 2256 |
| CCR5-2851 | − | GGAGAAAAAGGGGACAC | 17 | 2257 |
| CCR5-2852 | + | GAUUUGUACAAGAUCAC | 17 | 2258 |
| CCR5-2853 | + | UUCAGAAGGCAUCUCAC | 17 | 2259 |
| CCR5-2854 | − | GGUGGGAUAGGGGAUAC | 17 | 2260 |
| CCR5-2855 | + | GCUGAGAGGUUACUUAC | 17 | 2261 |
| CCR5-2856 | + | UCUGAAGGUUUAUUUAC | 17 | 2262 |
| CCR5-2857 | − | UGAGUAAAAGACUUUAC | 17 | 2263 |
| CCR5-2858 | + | CUGAGAGGUUACUUACC | 17 | 2264 |
| CCR5-2859 | − | CUACAAGAAACUCUCCC | 17 | 2265 |
| CCR5-2860 | + | AAUGUAGAGGGGGAUCC | 17 | 2266 |
| CCR5-2861 | − | GGGUUAAUGUGAAGUCC | 17 | 2267 |
| CCR5-2862 | − | GAUUUGCACAGCUCAUC | 17 | 2268 |
| CCR5-2863 | + | GCUAGAGAAUAGAUCUC | 17 | 2269 |
| CCR5-2864 | + | GGAUGUCUCAGCUCUUC | 17 | 2270 |
| CCR5-2865 | − | GGAGAGUGGAGAAAAAG | 17 | 2271 |
| CCR5-2866 | − | AGGGGAUACGGGGAGAG | 17 | 2272 |
| CCR5-2867 | + | CAACUUUAAAUGUAGAG | 17 | 2273 |
| CCR5-2868 | + | AAGGCAUCUCACUGGAG | 17 | 2274 |
| CCR5-2869 | + | CAGGCCAAGCAGCUGAG | 17 | 2275 |
| CCR5-2870 | + | CAAAUCUUUCUUUUGAG | 17 | 2276 |
| CCR5-2871 | − | GGGUUGGGGUGGGAUAG | 17 | 2277 |
| CCR5-2872 | + | ACCAACUUUAAAUGUAG | 17 | 2278 |
| CCR5-2873 | − | UAACAGAUUCUGUGUAG | 17 | 2279 |
| CCR5-2874 | + | GGGAGAGUUUCUUGUAG | 17 | 2280 |
| CCR5-2875 | − | GUGGGAUAGGGGAUACG | 17 | 2281 |
| CCR5-2876 | + | GCUGUUUCUUUUGAAGG | 17 | 2282 |
| CCR5-2877 | + | AACUUUAAAUGUAGAGG | 17 | 2283 |
| CCR5-2878 | + | UUUCUUUUGAAGGAGGG | 17 | 2284 |
| CCR5-2879 | − | CUGUGUGGGGGUUGGGG | 17 | 2285 |
| CCR5-2880 | − | AGAACAAUAAUAUUGGG | 17 | 2286 |
| CCR5-2881 | − | GGUGAGCAUCUGUGUGG | 17 | 2287 |
| CCR5-2882 | − | UUUCUUUUACUAAAAUG | 17 | 2288 |
| CCR5-2883 | − | GGUGGUGAGCAUCUGUG | 17 | 2289 |
| CCR5-2884 | − | UGGUGAGCAUCUGUGUG | 17 | 2290 |
| CCR5-2885 | − | CAUCUGUGUGGGGGUUG | 17 | 2291 |
| CCR5-2886 | − | GGGGGUUGGGGUGGGAU | 17 | 2292 |
| CCR5-2887 | − | ACAGAGAACAAUAAUAU | 17 | 2293 |
| CCR5-2888 | + | UGCCAAAUGUCUUCUAU | 17 | 2294 |
| CCR5-2889 | + | AUAAUUGUAUGAGCACU | 17 | 2295 |
| CCR5-2890 | − | GUAACCUCUCAGCUGCU | 17 | 2296 |
| CCR5-2891 | − | ACAAAUCAUUUGCUUCU | 17 | 2297 |
| CCR5-2892 | + | AUAGACAGUAUAAAAGU | 17 | 2298 |
| CCR5-2893 | − | CCCUCUACAUUUAAAGU | 17 | 2299 |
| CCR5-2894 | − | UUAAAGUUGGUUUAAGU | 17 | 2300 |
| CCR5-2895 | − | AACAGAUUCUGUGUAGU | 17 | 2301 |
| CCR5-2896 | − | AGCAUCUGUGUGGGGGU | 17 | 2302 |
| CCR5-2897 | − | UGUGUGGGGGUUGGGGU | 17 | 2303 |
| CCR5-2898 | − | UUCUUUUACUAAAAUGU | 17 | 2304 |
| CCR5-2899 | − | GUGGUGAGCAUCUGUGU | 17 | 2305 |
| CCR5-2900 | + | CGGGGAGAGUUUCUUGU | 17 | 2306 |
| CCR5-2901 | − | AACCCAUAGAAGACAUU | 17 | 2307 |
| CCR5-2902 | − | CAGAGAACAAUAAUAUU | 17 | 2308 |
| CCR5-2903 | − | AGGAAAGGGUCACAGUU | 17 | 2309 |
| CCR5-2904 | − | GCAUCUGUGUGGGGGUU | 17 | 2310 |
| CCR5-2905 | − | ACGGGGAGAGUGGAGAAAAA | 20 | 2311 |
| CCR5-2906 | − | UACGGGGAGAGUGGAGAAAA | 20 | 2312 |
| CCR5-2907 | − | UAAUCUUUAAGAUAAGGAAA | 20 | 2313 |
| CCR5-2908 | + | UUUUCAACAGUAAGGCUAAA | 20 | 2314 |
| CCR5-2909 | − | UGUGAGUGAAAGACUUUAAA | 20 | 2315 |
| CCR5-2910 | − | AUAAUCUUUAAGAUAAGGAA | 20 | 2316 |
| CCR5-2911 | + | GAGAGUUUCUUGUAGGGGAA | 20 | 2317 |
| CCR5-2912 | + | UUAGAAAAUAUAAAGAAUAA | 20 | 2318 |
| CCR5-2913 | − | UUGUGAGUGAAAGACUUUAA | 20 | 2319 |
| CCR5-2914 | − | GUGGAGAAAAAGGGGACACA | 20 | 2320 |
| CCR5-2915 | + | AUGAUUUGUACAAGAUCACA | 20 | 2321 |
| CCR5-2916 | − | AGUUUGGAAUGAGUUUCAGA | 20 | 2322 |
| CCR5-2917 | + | AGAAGGCAUCUCACUGGAGA | 20 | 2323 |
| CCR5-2918 | + | AAACCAACUUUAAAUGUAGA | 20 | 2324 |
| CCR5-2919 | + | AUGCUGUUUCUUUUGAAGGA | 20 | 2325 |
| CCR5-2920 | + | UAAAUCAUUAAGUGUAUUGA | 20 | 2326 |
| CCR5-2921 | + | GGAAAUGCUGUUUCUUUUGA | 20 | 2327 |
| CCR5-2922 | − | AAAAUAUAAUCUUUAAGAUA | 20 | 2328 |
| CCR5-2923 | − | UUGGGGUGGGAUAGGGGAUA | 20 | 2329 |
| CCR5-2924 | − | GUGGGGGUUGGGGUGGGAUA | 20 | 2330 |
| CCR5-2925 | − | UGAAAUCUUAUCUUCUGCUA | 20 | 2331 |
| CCR5-2926 | + | UGUUUGCCAAAUGUCUUCUA | 20 | 2332 |
| CCR5-2927 | + | CACAGGGCUUUUCAACAGUA | 20 | 2333 |
| CCR5-2928 | + | AAUCUUUCUUUUGAGAGGUA | 20 | 2334 |
| CCR5-2929 | + | ACCGGGGAGAGUUUCUUGUA | 20 | 2335 |
| CCR5-2930 | − | AGUGGAGAAAAAGGGGACAC | 20 | 2336 |
| CCR5-2931 | + | AAUGAUUUGUACAAGAUCAC | 20 | 2337 |
| CCR5-2932 | + | AUAUUCAGAAGGCAUCUCAC | 20 | 2338 |
| CCR5-2933 | + | UAUUUACGGGCUUUUCUCAC | 20 | 2339 |
| CCR5-2934 | − | UGGGGUGGGAUAGGGGAUAC | 20 | 2340 |
| CCR5-2935 | + | GCAGCUGAGAGGUUACUUAC | 20 | 2341 |
| CCR5-2936 | − | AGAUGAGUAAAAGACUUUAC | 20 | 2342 |
| CCR5-2937 | + | CAGCUGAGAGGUUACUUACC | 20 | 2343 |
| CCR5-2938 | + | UUAAAUGUAGAGGGGGAUCC | 20 | 2344 |
| CCR5-2939 | − | ACAGGGUUAAUGUGAAGUCC | 20 | 2345 |
| CCR5-2940 | − | AUUGAUUUGCACAGCUCAUC | 20 | 2346 |
| CCR5-2941 | + | UAAGCUAGAGAAUAGAUCUC | 20 | 2347 |
| CCR5-2942 | + | AACGGAUGUCUCAGCUCUUC | 20 | 2348 |
| CCR5-2943 | − | CGGGGAGAGUGGAGAAAAAG | 20 | 2349 |
| CCR5-2944 | + | AACCAACUUUAAAUGUAGAG | 20 | 2350 |
| CCR5-2945 | + | CAGAAGGCAUCUCACUGGAG | 20 | 2351 |
| CCR5-2946 | + | UAACAGGCCAAGCAGCUGAG | 20 | 2352 |
| CCR5-2947 | + | CUGCAAAUCUUUCUUUUGAG | 20 | 2353 |
| CCR5-2948 | − | UGGGGGUUGGGGUGGGAUAG | 20 | 2354 |
| CCR5-2949 | + | UAAACCAACUUUAAAUGUAG | 20 | 2355 |
| CCR5-2950 | − | UUCUAACAGAUUCUGUGUAG | 20 | 2356 |
| CCR5-2951 | − | GGGGUGGGAUAGGGGAUACG | 20 | 2357 |
| CCR5-2952 | + | AAUGCUGUUUCUUUUGAAGG | 20 | 2358 |
| CCR5-2953 | + | ACCAACUUUAAAUGUAGAGG | 20 | 2359 |
| CCR5-2954 | + | CUGUUUCUUUUGAAGGAGGG | 20 | 2360 |
| CCR5-2955 | − | CAUCUGUGUGGGGGUUGGGG | 20 | 2361 |
| CCR5-2956 | − | CAGAGAACAAUAAUAUUGGG | 20 | 2362 |
| CCR5-2957 | − | GGUGGUGAGCAUCUGUGUGG | 20 | 2363 |
| CCR5-2958 | − | UAAUUUCUUUUACUAAAAUG | 20 | 2364 |
| CCR5-2959 | − | UUGGGUGGUGAGCAUCUGUG | 20 | 2365 |
| CCR5-2960 | − | GGGUGGUGAGCAUCUGUGUG | 20 | 2366 |
| CCR5-2961 | − | GAGCAUCUGUGUGGGGGUUG | 20 | 2367 |
| CCR5-2962 | − | UGUGGGGGUUGGGGUGGGAU | 20 | 2368 |
| CCR5-2963 | − | UUUACAGAGAACAAUAAUAU | 20 | 2369 |
| CCR5-2964 | + | GUUUGCCAAAUGUCUUCUAU | 20 | 2370 |
| CCR5-2965 | − | UAAGUAACCUCUCAGCUGCU | 20 | 2371 |
| CCR5-2966 | − | UGUACAAAUCAUUUGCUUCU | 20 | 2372 |
| CCR5-2967 | + | CAUAUAGACAGUAUAAAAGU | 20 | 2373 |
| CCR5-2968 | − | CAUUUAAAGUUGGUUUAAGU | 20 | 2374 |
| CCR5-2969 | − | UCUAACAGAUUCUGUGUAGU | 20 | 2375 |
| CCR5-2970 | − | GUGAGCAUCUGUGUGGGGGU | 20 | 2376 |
| CCR5-2971 | − | AUCUGUGUGGGGGUUGGGGU | 20 | 2377 |
| CCR5-2972 | − | AAUUUCUUUUACUAAAAUGU | 20 | 2378 |
| CCR5-2973 | − | UGGGUGGUGAGCAUCUGUGU | 20 | 2379 |
| CCR5-2974 | + | UACCGGGGAGAGUUUCUUGU | 20 | 2380 |
| CCR5-2975 | − | GGAAACCCAUAGAAGACAUU | 20 | 2381 |
| CCR5-2976 | − | UUACAGAGAACAAUAAUAUU | 20 | 2382 |
| CCR5-2977 | − | AUAAGGAAAGGGUCACAGUU | 20 | 2383 |
| CCR5-2978 | − | UGAGCAUCUGUGUGGGGGUU | 20 | 2384 |

Table 5C provides exemplary targeting domains for knocking down the CCR5 gene selected according to the third tier parameters. Within the additional 500 bp (e.g., upstream or downstream) of a transcription start site (TSS), e.g., extending to 1kb upstream and downstream of a TSS. It is contemplated herein that in an embodiment the targeting domain hybridizes to the target domain through complementary base pairing. Any of the targeting domains in the table can be used with a S. pyogenes eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 5C
3rd Tier

| | | | Target | |
|---|---|---|---|---|
| gRNA | DNA | | Site | SEQ ID |
| Name | Strand | Targeting Domain | Length | NO |
| CCR5-2979 | − | AGAGGGAAGCCUAAAAA | 17 | 2385 |
| CCR5-2980 | + | AUGCUUACUGGUUUGAA | 17 | 2386 |
| CCR5-2981 | − | GGAGUUUGAGACUCACA | 17 | 2387 |
| CCR5-2982 | + | UUUUUAUUCUAGAGCCA | 17 | 2388 |
| CCR5-2983 | − | GCCUAGUCUAAGGUGCA | 17 | 2389 |
| CCR5-2984 | − | UUUUAACUAUGGGCUCA | 17 | 2390 |
| CCR5-2985 | + | UUCUAGAGCCAAGGUCA | 17 | 2391 |
| CCR5-2986 | − | CUAAUAUAUCAGUUUCA | 17 | 2392 |
| CCR5-2987 | + | CUGGGUCCAGAAAAAGA | 17 | 2393 |
| CCR5-2988 | − | UUUUCCUCCAGACAAGA | 17 | 2394 |
| CCR5-2989 | − | GCUUGUGAUCUCUAAGA | 17 | 2395 |
| CCR5-2990 | + | GGUCACGGAAGCCCAGA | 17 | 2396 |
| CCR5-2991 | + | AAUGCUUACUGGUUUGA | 17 | 2397 |
| CCR5-2992 | − | CACAUGACAUAAGUAUA | 17 | 2398 |
| CCR5-2993 | − | CUAAAGAGUUUUAACUA | 17 | 2399 |
| CCR5-2994 | − | CUCAGCUGCCUAGUCUA | 17 | 2400 |
| CCR5-2995 | − | AAAAAUGAGCUUUUCUA | 17 | 2401 |
| CCR5-2996 | − | UAGUAUAUAAUUCUUUA | 17 | 2402 |
| CCR5-2997 | − | UCACGGGUGAGCUAAAC | 17 | 2403 |
| CCR5-2998 | + | AAAACUCUUUAGACAAC | 17 | 2404 |
| CCR5-2999 | − | GGGAGUUUGAGACUCAC | 17 | 2405 |
| CCR5-3000 | − | UUUAACUAUGGGCUCAC | 17 | 2406 |
| CCR5-3001 | + | UCCUCAUAAAUGCUUAC | 17 | 2407 |
| CCR5-3002 | − | CAUCUUUUUCUGGACCC | 17 | 2408 |
| CCR5-3003 | − | UCAUCUAUGACCUUCCC | 17 | 2409 |
| CCR5-3004 | + | AAUCCCCACUAAGAUCC | 17 | 2410 |
| CCR5-3005 | − | AGACUAGGCAAGACAGC | 17 | 2411 |
| CCR5-3006 | − | CCAGAUACAUAGGUGGC | 17 | 2412 |
| CCR5-3007 | − | UGCCUAGUCUAAGGUGC | 17 | 2413 |
| CCR5-3008 | + | UUCAGAUAGAUUAUAUC | 17 | 2414 |
| CCR5-3009 | + | CCUGCCACCUAUGUAUC | 17 | 2415 |
| CCR5-3010 | − | AGCCACAAGAUGCCCUC | 17 | 2416 |
| CCR5-3011 | + | AGGGCAUCUUGUGGCUC | 17 | 2417 |
| CCR5-3012 | − | GAAGUUGUGUCUAAGUC | 17 | 2418 |
| CCR5-3013 | + | UAGGCUUCCCUCUUGUC | 17 | 2419 |
| CCR5-3014 | + | AUGAAUGUCAUGCAUUC | 17 | 2420 |
| CCR5-3015 | − | AGUAUAUGGUCAAGUUC | 17 | 2421 |
| CCR5-3016 | − | GGUUUCCCAUCUUUUUC | 17 | 2422 |
| CCR5-3017 | − | UUUUUCCUCCAGACAAG | 17 | 2423 |
| CCR5-3018 | − | UGCCCCCAAUCCUACAG | 17 | 2424 |
| CCR5-3019 | + | AGGUCACGGAAGCCCAG | 17 | 2425 |
| CCR5-3020 | − | AAAAUGAGCUUUUCUAG | 17 | 2426 |
| CCR5-3021 | + | UGAAACUGAUAUAUUAG | 17 | 2427 |
| CCR5-3022 | − | UGGACCCAGGAUCUUAG | 17 | 2428 |
| CCR5-3023 | − | UAUGCCAGAUACAUAGG | 17 | 2429 |
| CCR5-3024 | + | GCUUCCCUCUUGUCUGG | 17 | 2430 |
| CCR5-3025 | − | AUGACAUUCAUCUGUGG | 17 | 2431 |
| CCR5-3026 | + | UGCCUCUGUAGGAUUGG | 17 | 2432 |
| CCR5-3027 | − | AUAUCAAGCUCUCUUGG | 17 | 2433 |
| CCR5-3028 | + | CAUAUACUUAUGUCAUG | 17 | 2434 |
| CCR5-3029 | − | ACCAGUAAGCAUUUAUG | 17 | 2435 |
| CCR5-3030 | − | UGCAUGACAUUCAUCUG | 17 | 2436 |
| CCR5-3031 | − | GACCCAGGAUCUUAGUG | 17 | 2437 |
| CCR5-3032 | − | ACUUCACAGAAAAUGUG | 17 | 2438 |
| CCR5-3033 | − | AUGACAACUCUUAAUUG | 17 | 2439 |
| CCR5-3034 | + | CUGCCUCUGUAGGAUUG | 17 | 2440 |
| CCR5-3035 | + | GCCCAGAGGGCAUCUUG | 17 | 2441 |
| CCR5-3036 | + | UUAGACACAACUUCUUG | 17 | 2442 |
| CCR5-3037 | + | CGUAAUUUUGCUGUUUG | 17 | 2443 |
| CCR5-3038 | − | UGUGAGGAUUUUACAAU | 17 | 2444 |
| CCR5-3039 | − | CACUAUGCCAGAUACAU | 17 | 2445 |
| CCR5-3040 | + | UGGGUCCAGAAAAAGAU | 17 | 2446 |
| CCR5-3041 | − | UAAAGAGUUUUAACUAU | 17 | 2447 |
| CCR5-3042 | − | CUGAACUUAAAUAGACU | 17 | 2448 |
| CCR5-3043 | + | UCCCUGCACCUUAGACU | 17 | 2449 |
| CCR5-3044 | − | CUGGGCUUCCGUGACCU | 17 | 2450 |
| CCR5-3045 | − | CAUCUAUGACCUUCCCU | 17 | 2451 |
| CCR5-3046 | + | AUCCCCACUAAGAUCCU | 17 | 2452 |
| CCR5-3047 | + | GAGGGCAUCUUGUGGCU | 17 | 2453 |
| CCR5-3048 | − | GCCACAAGAUGCCCUCU | 17 | 2454 |
| CCR5-3049 | − | GUCAUAUCAAGCUCUCU | 17 | 2455 |
| CCR5-3050 | + | UGAAUGUCAUGCAUUCU | 17 | 2456 |
| CCR5-3051 | − | UUUAUUAUAUUAUUUCU | 17 | 2457 |
| CCR5-3052 | − | UAAAAAUGAGCUUUUCU | 17 | 2458 |
| CCR5-3053 | − | GGACCCAGGAUCUUAGU | 17 | 2459 |
| CCR5-3054 | − | CAAGCUCUCUUGGCGGU | 17 | 2460 |
| CCR5-3055 | + | UAGACACAACUUCUUGU | 17 | 2461 |
| CCR5-3056 | + | UCUGCCUCUGUAGGAUU | 17 | 2462 |
| CCR5-3057 | + | UAGAGGAAAAUUUUAUU | 17 | 2463 |
| CCR5-3058 | − | UCUAGAAUAAAAAGCUU | 17 | 2464 |
| CCR5-3059 | − | UUAUUAUAUUAUUUCUU | 17 | 2465 |
| CCR5-3060 | + | CACGUAAUUUUGCUGUU | 17 | 2466 |
| CCR5-3061 | + | ACGUAAUUUUGCUGUUU | 17 | 2467 |
| CCR5-3062 | + | UAAUUUUGACCAUUUUU | 17 | 2468 |
| CCR5-3063 | − | ACAAGAGGGAAGCCUAAAAA | 20 | 2469 |
| CCR5-3064 | + | UAAAUGCUUACUGGUUUGAA | 20 | 2470 |
| CCR5-3065 | − | CAGGGAGUUUGAGACUCACA | 20 | 2471 |
| CCR5-3066 | + | AGCUUUUUAUUCUAGAGCCA | 20 | 2472 |
| CCR5-3067 | − | GCUGCCUAGUCUAAGGUGCA | 20 | 2473 |
| CCR5-3068 | − | GAGUUUUAACUAUGGGCUCA | 20 | 2474 |
| CCR5-3069 | + | UUAUUCUAGAGCCAAGGUCA | 20 | 2475 |
| CCR5-3070 | − | CCUCUAAUAUAUCAGUUUCA | 20 | 2476 |
| CCR5-3071 | + | AUCCUGGGUCCAGAAAAAGA | 20 | 2477 |
| CCR5-3072 | − | UCUUUUUCCUCCAGACAAGA | 20 | 2478 |
| CCR5-3073 | − | UUGGCUUGUGAUCUCUAAGA | 20 | 2479 |
| CCR5-3074 | + | CAAGGUCACGGAAGCCCAGA | 20 | 2480 |
| CCR5-3075 | + | AUAAAUGCUUACUGGUUUGA | 20 | 2481 |
| CCR5-3076 | − | UUCCACAUGACAUAAGUAUA | 20 | 2482 |
| CCR5-3077 | − | UGUCUAAAGAGUUUUAACUA | 20 | 2483 |
| CCR5-3078 | − | UCUCUCAGCUGCCUAGUCUA | 20 | 2484 |
| CCR5-3079 | − | AUUAAAAAUGAGCUUUUCUA | 20 | 2485 |
| CCR5-3080 | − | AGUUAGUAUAUAAUUCUUUA | 20 | 2486 |
| CCR5-3081 | − | GGCUCACGGGUGAGCUAAAC | 20 | 2487 |
| CCR5-3082 | + | GUUAAAACUCUUUAGACAAC | 20 | 2488 |
| CCR5-3083 | − | GCAGGGAGUUUGAGACUCAC | 20 | 2489 |
| CCR5-3084 | − | AGUUUUAACUAUGGGCUCAC | 20 | 2490 |
| CCR5-3085 | + | GAGUCCUCAUAAAUGCUUAC | 20 | 2491 |
| CCR5-3086 | − | UCCCAUCUUUUUCUGGACCC | 20 | 2492 |
| CCR5-3087 | − | UUGUCAUCUAUGACCUUCCC | 20 | 2493 |
| CCR5-3088 | + | GAAAAUCCCCACUAAGAUCC | 20 | 2494 |
| CCR5-3089 | − | AAUAGACUAGGCAAGACAGC | 20 | 2495 |
| CCR5-3090 | − | AUGCCAGAUACAUAGGUGGC | 20 | 2496 |
| CCR5-3091 | − | AGCUGCCUAGUCUAAGGUGC | 20 | 2497 |
| CCR5-3092 | + | AGCUUCAGAUAGAUUAUAUC | 20 | 2498 |
| CCR5-3093 | + | AAUCCUGCCACCUAUGUAUC | 20 | 2499 |
| CCR5-3094 | − | CCGAGCCACAAGAUGCCCUC | 20 | 2500 |
| CCR5-3095 | + | CAGAGGGCAUCUUGUGGCUC | 20 | 2501 |
| CCR5-3096 | − | CAAGAAGUUGUGUCUAAGUC | 20 | 2502 |
| CCR5-3097 | + | UUUUAGGCUUCCCUCUUGUC | 20 | 2503 |
| CCR5-3098 | + | CAGAUGAAUGUCAUGCAUUC | 20 | 2504 |
| CCR5-3099 | − | AUAAGUAUAUGGUCAAGUUC | 20 | 2505 |
| CCR5-3100 | − | ACAGGUUUCCCAUCUUUUUC | 20 | 2506 |
| CCR5-3101 | − | UUCUUUUUCCUCCAGACAAG | 20 | 2507 |
| CCR5-3102 | − | ACGUGCCCCCAAUCCUACAG | 20 | 2508 |
| CCR5-3103 | + | CCAAGGUCACGGAAGCCCAG | 20 | 2509 |
| CCR5-3104 | − | UUAAAAAUGAGCUUUUCUAG | 20 | 2510 |
| CCR5-3105 | + | CCAUGAAACUGAUAUAUUAG | 20 | 2511 |
| CCR5-3106 | − | UUCUGGACCCAGGAUCUUAG | 20 | 2512 |
| CCR5-3107 | − | CACUAUGCCAGAUACAUAGG | 20 | 2513 |
| CCR5-3108 | + | UAGGCUUCCCUCUUGUCUGG | 20 | 2514 |
| CCR5-3109 | − | UGCAUGACAUUCAUCUGUGG | 20 | 2515 |
| CCR5-3110 | − | GUCAUAUCAAGCUCUCUUGG | 20 | 2516 |
| CCR5-3111 | + | GACCAUAUACUUAUGUCAUG | 20 | 2517 |
| CCR5-3112 | − | CAAACCAGUAAGCAUUUAUG | 20 | 2518 |
| CCR5-3113 | − | GAAUGCAUGACAUUCAUCUG | 20 | 2519 |
| CCR5-3114 | − | CUGGACCCAGGAUCUUAGUG | 20 | 2520 |
| CCR5-3115 | − | CAAACUUCACAGAAAAUGUG | 20 | 2521 |
| CCR5-3116 | − | UGUAUGACAACUCUUAAUUG | 20 | 2522 |
| CCR5-3117 | + | GAAGCCCAGAGGGCAUCUUG | 20 | 2523 |
| CCR5-3118 | + | GACUUAGACACAACUUCUUG | 20 | 2524 |
| CCR5-3119 | + | GCACGUAAUUUUGCUGUUUG | 20 | 2525 |
| CCR5-3120 | − | AAAUGUGAGGAUUUUACAAU | 20 | 2526 |
| CCR5-3121 | − | UCACACUAUGCCAGAUACAU | 20 | 2527 |
| CCR5-3122 | + | UCCUGGGUCCAGAAAAAGAU | 20 | 2528 |
| CCR5-3123 | − | GUCUAAAGAGUUUUAACUAU | 20 | 2529 |
| CCR5-3124 | − | CAGCUGAACUUAAAUAGACU | 20 | 2530 |
| CCR5-3125 | + | AACUCCCUGCACCUUAGACU | 20 | 2531 |
| CCR5-3126 | − | CCUCUGGGCUUCCGUGACCU | 20 | 2532 |
| CCR5-3127 | − | UGUCAUCUAUGACCUUCCCU | 20 | 2533 |
| CCR5-3128 | + | AAAAUCCCCACUAAGAUCCU | 20 | 2534 |
| CCR5-3129 | + | CCAGAGGGCAUCUUGUGGCU | 20 | 2535 |
| CCR5-3130 | − | CGAGCCACAAGAUGCCCUCU | 20 | 2536 |
| CCR5-3131 | − | ACAGUCAUAUCAAGCUCUCU | 20 | 2537 |
| CCR5-3132 | + | AGAUGAAUGUCAUGCAUUCU | 20 | 2538 |
| CCR5-3133 | − | UUUUUUAUUAUAUUAUUUCU | 20 | 2539 |
| CCR5-3134 | − | AAUUAAAAAUGAGCUUUUCU | 20 | 2540 |
| CCR5-3135 | − | UCUGGACCCAGGAUCUUAGU | 20 | 2541 |
| CCR5-3136 | − | UAUCAAGCUCUCUUGGCGGU | 20 | 2542 |
| CCR5-3137 | + | ACUUAGACACAACUUCUUGU | 20 | 2543 |
| CCR5-3138 | + | UAUUAGAGGAAAAUUUUAUU | 20 | 2544 |
| CCR5-3139 | − | GGCUCUAGAAUAAAAAGCUU | 20 | 2545 |
| CCR5-3140 | − | UUUUUAUUAUAUUAUUUCUU | 20 | 2546 |
| CCR5-3141 | + | GGGCACGUAAUUUUGCUGUU | 20 | 2547 |
| CCR5-3142 | + | GGCACGUAAUUUUGCUGUUU | 20 | 2548 |
| CCR5-3143 | + | UAUUAAUUUUGACCAUUUUU | 20 | 2549 |

Table 6A provides exemplary targeting domains for knocking down the CCR5 gene selected according to the first tier parameters. The targeting domains bind within 500 bp (e.g., upstream or downstream) of a transcription start site (TSS), have a high level of orthogonality and PAM is NNGRRT. It is contemplated herein that in an embodiment the targeting domain hybridizes to the target domain through complementary base pairing. Any of the targeting domains in the table can be used with a S. aureus eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 6A
1st Tier

| gRNA | DNA | | Target Site | SEQ ID |
|---|---|---|---|---|
| Name | Strand | Targeting Domain | Length | NO |
| CCR5-3144 | + | AAGUGUAUUGAAGGCGAA | 18 | 2550 |
| CCR5-3145 | + | UAAGUGUAUUGAAGGCGAA | 19 | 2551 |
| CCR5-3146 | + | UUAAGUGUAUUGAAGGCGAA | 20 | 2552 |
| CCR5-3147 | + | AUUAAGUGUAUUGAAGGCGAA | 21 | 2553 |
| CCR5-3148 | + | CAUUAAGUGUAUUGAAGGCGAA | 22 | 2554 |
| CCR5-3149 | + | UCAUUAAGUGUAUUGAAGGCGAA | 23 | 2555 |
| CCR5-3150 | + | AUCAUUAAGUGUAUUGAAGGCGAA | 24 | 2556 |
| CCR5-3151 | + | UUCUCUGCUCAUCCCACUACA | 21 | 2557 |
| CCR5-3152 | + | GUUCUCUGCUCAUCCCACUACA | 22 | 2558 |
| CCR5-3153 | + | UGUUCUCUGCUCAUCCCACUACA | 23 | 2559 |
| CCR5-3154 | + | UUGUUCUCUGCUCAUCCCACUACA | 24 | 2560 |
| CCR5-3155 | + | AUUUACGGGCUUUUCUCA | 18 | 2561 |
| CCR5-3156 | + | UAUUUACGGGCUUUUCUCA | 19 | 2562 |
| CCR5-3157 | + | UUAUUUACGGGCUUUUCUCA | 20 | 2563 |
| CCR5-3158 | + | UUUAUUUACGGGCUUUUCUCA | 21 | 2564 |
| CCR5-3159 | + | GUUUAUUUACGGGCUUUUCUCA | 22 | 2565 |
| CCR5-3160 | + | GGUUUAUUUACGGGCUUUUCUCA | 23 | 2566 |
| CCR5-3161 | + | AGGUUUAUUUACGGGCUUUUCUCA | 24 | 2567 |
| CCR5-3162 | + | GGGAGAGUUUCUUGUAGGGGA | 21 | 2568 |
| CCR5-3163 | + | GGGGAGAGUUUCUUGUAGGGGA | 22 | 2569 |
| CCR5-3164 | + | CGGGGAGAGUUUCUUGUAGGGGA | 23 | 2570 |
| CCR5-3165 | + | CCGGGGAGAGUUUCUUGUAGGGGA | 24 | 2571 |
| CCR5-3166 | + | UUCAGAAGGCAUCUCACUGGA | 21 | 2572 |
| CCR5-3167 | + | AUUCAGAAGGCAUCUCACUGGA | 22 | 2573 |
| CCR5-3168 | + | UAUUCAGAAGGCAUCUCACUGGA | 23 | 2574 |
| CCR5-3169 | + | AUAUUCAGAAGGCAUCUCACUGGA | 24 | 2575 |
| CCR5-3170 | + | UGAGCUUAAAAUAAGCUA | 18 | 2576 |
| CCR5-3171 | + | UUGAGCUUAAAAUAAGCUA | 19 | 2577 |
| CCR5-3172 | + | GUUGAGCUUAAAAUAAGCUA | 20 | 2578 |
| CCR5-3173 | + | GAAAUGCUGUUUCUUUUGAAG | 21 | 2579 |
| CCR5-3174 | + | GGAAAUGCUGUUUCUUUUGAAG | 22 | 2580 |
| CCR5-3175 | + | AGGAAAUGCUGUUUCUUUUGAAG | 23 | 2581 |
| CCR5-3176 | + | UAGGAAAUGCUGUUUCUUUUGAAG | 24 | 2582 |
| CCR5-3177 | + | AAACCAACUUUAAAUGUAGAG | 21 | 2583 |
| CCR5-3178 | + | UAAACCAACUUUAAAUGUAGAG | 22 | 2584 |
| CCR5-3179 | + | UUAAACCAACUUUAAAUGUAGAG | 23 | 2585 |
| CCR5-3180 | + | CUUAAACCAACUUUAAAUGUAGAG | 24 | 2586 |
| CCR5-3181 | + | GCUGUUUCUUUUGAAGGAGGG | 21 | 2587 |
| CCR5-3182 | + | UGCUGUUUCUUUUGAAGGAGGG | 22 | 2588 |
| CCR5-3183 | + | AUGCUGUUUCUUUUGAAGGAGGG | 23 | 2589 |
| CCR5-3184 | + | AAUGCUGUUUCUUUUGAAGGAGGG | 24 | 2590 |
| CCR5-3185 | + | GCUGAGAGGUUACUUACCGGG | 21 | 2591 |
| CCR5-3186 | + | AGCUGAGAGGUUACUUACCGGG | 22 | 2592 |
| CCR5-3187 | + | CAGCUGAGAGGUUACUUACCGGG | 23 | 2593 |
| CCR5-3188 | + | GCAGCUGAGAGGUUACUUACCGGG | 24 | 2594 |
| CCR5-3189 | + | CAAAUCUUUCUUUUGAGAGGU | 21 | 2595 |
| CCR5-3190 | + | GCAAAUCUUUCUUUUGAGAGGU | 22 | 2596 |
| CCR5-3191 | + | UGCAAAUCUUUCUUUUGAGAGGU | 23 | 2597 |
| CCR5-3192 | + | CUGCAAAUCUUUCUUUUGAGAGGU | 24 | 2598 |
| CCR5-3193 | − | AGGAAAGGGUCACAGUUUGGA | 21 | 2599 |
| CCR5-3194 | − | AAGGAAAGGGUCACAGUUUGGA | 22 | 2600 |
| CCR5-3195 | − | UAAGGAAAGGGUCACAGUUUGGA | 23 | 2601 |
| CCR5-3196 | − | AUAAGGAAAGGGUCACAGUUUGGA | 24 | 2602 |
| CCR5-3197 | − | ACACAGGGUUAAUGUGAAGUC | 21 | 2603 |
| CCR5-3198 | − | GACACAGGGUUAAUGUGAAGUC | 22 | 2604 |
| CCR5-3199 | − | GGACACAGGGUUAAUGUGAAGUC | 23 | 2605 |
| CCR5-3200 | − | GGGACACAGGGUUAAUGUGAAGUC | 24 | 2606 |
| CCR5-3201 | − | GCCUGUUAGUUAGCUUCUGAG | 21 | 2607 |
| CCR5-3202 | − | GGCCUGUUAGUUAGCUUCUGAG | 22 | 2608 |
| CCR5-3203 | − | UGGCCUGUUAGUUAGCUUCUGAG | 23 | 2609 |
| CCR5-3204 | − | UUGGCCUGUUAGUUAGCUUCUGAG | 24 | 2610 |
| CCR5-3205 | − | AUGUGGGCUUUUGACUAG | 18 | 2611 |
| CCR5-3206 | − | AAUGUGGGCUUUUGACUAG | 19 | 2612 |
| CCR5-3207 | − | AAAUGUGGGCUUUUGACUAG | 20 | 2613 |
| CCR5-3208 | − | AAAAUGUGGGCUUUUGACUAG | 21 | 2614 |
| CCR5-3209 | − | UAAAAUGUGGGCUUUUGACUAG | 22 | 2615 |
| CCR5-3210 | − | CUAAAAUGUGGGCUUUUGACUAG | 23 | 2616 |
| CCR5-3211 | − | ACUAAAAUGUGGGCUUUUGACUAG | 24 | 2617 |
| CCR5-3212 | − | UUUCUAACAGAUUCUGUGUAG | 21 | 2618 |
| CCR5-3213 | − | UUUUCUAACAGAUUCUGUGUAG | 22 | 2619 |
| CCR5-3214 | − | AUUUUCUAACAGAUUCUGUGUAG | 23 | 2620 |
| CCR5-3215 | − | UAUUUUCUAACAGAUUCUGUGUAG | 24 | 2621 |
| CCR5-3216 | − | GGGUGGGAUAGGGGAUACGGG | 21 | 2622 |
| CCR5-3217 | − | GGGGUGGGAUAGGGGAUACGGG | 22 | 2623 |
| CCR5-3218 | − | UGGGGUGGGAUAGGGGAUACGGG | 23 | 2624 |
| CCR5-3219 | − | UUGGGGUGGGAUAGGGGAUACGGG | 24 | 2625 |
| CCR5-3220 | − | AGCAACUCUUAAGAUAAU | 18 | 2626 |
| CCR5-3221 | − | UAGCAACUCUUAAGAUAAU | 19 | 2627 |
| CCR5-3222 | − | AUAGCAACUCUUAAGAUAAU | 20 | 2628 |
| CCR5-3223 | − | AAUAGCAACUCUUAAGAUAAU | 21 | 2629 |
| CCR5-3224 | − | UAAUAGCAACUCUUAAGAUAAU | 22 | 2630 |
| CCR5-3225 | − | UUAAUAGCAACUCUUAAGAUAAU | 23 | 2631 |
| CCR5-3226 | − | AUUAAUAGCAACUCUUAAGAUAAU | 24 | 2632 |
| CCR5-3227 | − | GGUGAGCAUCUGUGUGGGGGU | 21 | 2633 |
| CCR5-3228 | − | UGGUGAGCAUCUGUGUGGGGGU | 22 | 2634 |
| CCR5-3229 | − | GUGGUGAGCAUCUGUGUGGGGGU | 23 | 2635 |
| CCR5-3230 | − | GGUGGUGAGCAUCUGUGUGGGGGU | 24 | 2636 |
| CCR5-3231 | − | UUGGGUGGUGAGCAUCUGUGU | 21 | 2637 |
| CCR5-3232 | − | AUUGGGUGGUGAGCAUCUGUGU | 22 | 2638 |
| CCR5-3233 | − | UAUUGGGUGGUGAGCAUCUGUGU | 23 | 2639 |
| CCR5-3234 | − | AUAUUGGGUGGUGAGCAUCUGUGU | 24 | 2640 |
| CCR5-3235 | − | UCAAAGAUACAAAACAUGAUU | 21 | 2641 |
| CCR5-3236 | − | AUCAAAGAUACAAAACAUGAUU | 22 | 2642 |
| CCR5-3237 | − | CAUCAAAGAUACAAAACAUGAUU | 23 | 2643 |
| CCR5-3238 | − | ACAUCAAAGAUACAAAACAUGAUU | 24 | 2644 |
| CCR5-3239 | − | CCCUCUCCAGUGAGAUGCCUU | 21 | 2645 |
| CCR5-3240 | − | ACCCUCUCCAGUGAGAUGCCUU | 22 | 2646 |
| CCR5-3241 | − | AACCCUCUCCAGUGAGAUGCCUU | 23 | 2647 |
| CCR5-3242 | − | AAACCCUCUCCAGUGAGAUGCCUU | 24 | 2648 |

Table 6B provides exemplary targeting domains for knocking down the CCR5 gene selected according to the second tier parameters. The targeting domains bind within 500 bp (e.g., upstream or downstream) of a transcription start site (TSS) and PAM is NNGRRT. It is contemplated herein that in an embodiment the targeting domain hybridizes to the target domain through complementary base pairing. Any of the targeting domains in the table can be used with a S. aureus eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 6B
2nd Tier

| gRNA | DNA | | Target Site | SEQ ID |
|---|---|---|---|---|
| Name | Strand | Targeting Domain | Length | NO |
| CCR5-3243 | + | UCUGCUCAUCCCACUACA | 18 | 2649 |
| CCR5-3244 | + | CUCUGCUCAUCCCACUACA | 19 | 2650 |
| CCR5-3245 | + | UCUCUGCUCAUCCCACUACA | 20 | 2651 |
| CCR5-3246 | + | AGAGUUUCUUGUAGGGGA | 18 | 2652 |
| CCR5-3247 | + | GAGAGUUUCUUGUAGGGGA | 19 | 2653 |
| CCR5-3248 | + | GGAGAGUUUCUUGUAGGGGA | 20 | 2654 |
| CCR5-3249 | + | AGAAGGCAUCUCACUGGA | 18 | 2655 |
| CCR5-3250 | + | CAGAAGGCAUCUCACUGGA | 19 | 2656 |
| CCR5-3251 | + | UCAGAAGGCAUCUCACUGGA | 20 | 2657 |
| CCR5-3252 | + | UAGAAAAUAUAAAGAAUA | 18 | 2658 |
| CCR5-3253 | + | UUAGAAAAUAUAAAGAAUA | 19 | 2659 |
| CCR5-3254 | + | GUUAGAAAAUAUAAAGAAUA | 20 | 2660 |
| CCR5-3255 | + | UGUUAGAAAAUAUAAAGAAUA | 21 | 2661 |
| CCR5-3256 | + | CUGUUAGAAAAUAUAAAGAAUA | 22 | 2662 |
| CCR5-3257 | + | UCUGUUAGAAAAUAUAAAGAAUA | 23 | 2663 |
| CCR5-3258 | + | AUCUGUUAGAAAAUAUAAAGAAUA | 24 | 2664 |
| CCR5-3259 | + | AAUCUGUUAGAAAAUAUA | 18 | 2665 |
| CCR5-3260 | + | GAAUCUGUUAGAAAAUAUA | 19 | 2666 |
| CCR5-3261 | + | AGAAUCUGUUAGAAAAUAUA | 20 | 2667 |
| CCR5-3262 | + | CAGAAUCUGUUAGAAAAUAUA | 21 | 2668 |
| CCR5-3263 | + | ACAGAAUCUGUUAGAAAAUAUA | 22 | 2669 |
| CCR5-3264 | + | CACAGAAUCUGUUAGAAAAUAUA | 23 | 2670 |
| CCR5-3265 | + | ACACAGAAUCUGUUAGAAAAUAUA | 24 | 2671 |
| CCR5-3266 | + | AGUUGAGCUUAAAAUAAGCUA | 21 | 2672 |
| CCR5-3267 | + | AAGUUGAGCUUAAAAUAAGCUA | 22 | 2673 |
| CCR5-3268 | + | UAAGUUGAGCUUAAAAUAAGCUA | 23 | 2674 |
| CCR5-3269 | + | UUAAGUUGAGCUUAAAAUAAGCUA | 24 | 2675 |
| CCR5-3270 | + | AUGCUGUUUCUUUUGAAG | 18 | 2676 |
| CCR5-3271 | + | AAUGCUGUUUCUUUUGAAG | 19 | 2677 |
| CCR5-3272 | + | AAAUGCUGUUUCUUUUGAAG | 20 | 2678 |
| CCR5-3273 | + | CCAACUUUAAAUGUAGAG | 18 | 2679 |
| CCR5-3274 | + | ACCAACUUUAAAUGUAGAG | 19 | 2680 |
| CCR5-2944 | + | AACCAACUUUAAAUGUAGAG | 20 | 2681 |
| CCR5-3275 | + | GUUUCUUUUGAAGGAGGG | 18 | 2682 |
| CCR5-3276 | + | UGUUUCUUUUGAAGGAGGG | 19 | 2683 |
| CCR5-2954 | + | CUGUUUCUUUUGAAGGAGGG | 20 | 2684 |
| CCR5-3277 | + | GAGAGGUUACUUACCGGG | 18 | 2685 |
| CCR5-3278 | + | UGAGAGGUUACUUACCGGG | 19 | 2686 |
| CCR5-3279 | + | CUGAGAGGUUACUUACCGGG | 20 | 2687 |
| CCR5-3280 | + | GUUUGCCAAAUGUCUUCU | 18 | 2688 |
| CCR5-3281 | + | UGUUUGCCAAAUGUCUUCU | 19 | 2689 |
| CCR5-3282 | + | GUGUUUGCCAAAUGUCUUCU | 20 | 2690 |
| CCR5-3283 | + | GGUGUUUGCCAAAUGUCUUCU | 21 | 2691 |
| CCR5-3284 | + | UGGUGUUUGCCAAAUGUCUUCU | 22 | 2692 |
| CCR5-3285 | + | UUGGUGUUUGCCAAAUGUCUUCU | 23 | 2693 |
| CCR5-3286 | + | CUUGGUGUUUGCCAAAUGUCUUCU | 24 | 2694 |
| CCR5-3287 | + | AUCUUUCUUUUGAGAGGU | 18 | 2695 |
| CCR5-3288 | + | AAUCUUUCUUUUGAGAGGU | 19 | 2696 |
| CCR5-3289 | + | AAAUCUUUCUUUUGAGAGGU | 20 | 2697 |
| CCR5-3290 | + | GAAAAUUCUGAUUAUCUU | 18 | 2698 |
| CCR5-3291 | + | AGAAAAUUCUGAUUAUCUU | 19 | 2699 |
| CCR5-3292 | + | AAGAAAAUUCUGAUUAUCUU | 20 | 2700 |
| CCR5-3293 | + | UAAGAAAAUUCUGAUUAUCUU | 21 | 2701 |
| CCR5-3294 | + | UUAAGAAAAUUCUGAUUAUCUU | 22 | 2702 |
| CCR5-3295 | + | GUUAAGAAAAUUCUGAUUAUCUU | 23 | 2703 |
| CCR5-3296 | + | GGUUAAGAAAAUUCUGAUUAUCUU | 24 | 2704 |
| CCR5-3297 | − | GUGGAGAAAAAGGGGACA | 18 | 2705 |
| CCR5-3298 | − | AGUGGAGAAAAAGGGGACA | 19 | 2706 |
| CCR5-3299 | − | GAGUGGAGAAAAAGGGGACA | 20 | 2707 |
| CCR5-3300 | − | AGAGUGGAGAAAAAGGGGACA | 21 | 2708 |
| CCR5-3301 | − | GAGAGUGGAGAAAAAGGGGACA | 22 | 2709 |
| CCR5-3302 | − | GGAGAGUGGAGAAAAAGGGGACA | 23 | 2710 |
| CCR5-3303 | − | GGGAGAGUGGAGAAAAAGGGGACA | 24 | 2711 |
| CCR5-3304 | − | UAAUCUUUAAGAUAAGGA | 18 | 2712 |
| CCR5-3305 | − | AUAAUCUUUAAGAUAAGGA | 19 | 2713 |
| CCR5-3306 | − | UAUAAUCUUUAAGAUAAGGA | 20 | 2714 |
| CCR5-3307 | − | AUAUAAUCUUUAAGAUAAGGA | 21 | 2715 |
| CCR5-3308 | − | AAUAUAAUCUUUAAGAUAAGGA | 22 | 2716 |
| CCR5-3309 | − | AAAUAUAAUCUUUAAGAUAAGGA | 23 | 2717 |
| CCR5-3310 | − | AAAAUAUAAUCUUUAAGAUAAGGA | 24 | 2718 |
| CCR5-3311 | − | AAAGGGUCACAGUUUGGA | 18 | 2719 |
| CCR5-3312 | − | GAAAGGGUCACAGUUUGGA | 19 | 2720 |
| CCR5-3313 | − | GGAAAGGGUCACAGUUUGGA | 20 | 2721 |
| CCR5-3314 | − | UUACAGAGAACAAUAAUA | 18 | 2722 |
| CCR5-3315 | − | UUUACAGAGAACAAUAAUA | 19 | 2723 |
| CCR5-3316 | − | GUUUACAGAGAACAAUAAUA | 20 | 2724 |
| CCR5-3317 | − | GGGGGUUGGGGUGGGAUA | 18 | 2725 |
| CCR5-3318 | − | UGGGGGUUGGGGUGGGAUA | 19 | 2726 |
| CCR5-2924 | − | GUGGGGGUUGGGGUGGGAUA | 20 | 2727 |
| CCR5-3319 | − | UGUGGGGGUUGGGGUGGGAUA | 21 | 2728 |
| CCR5-3320 | − | GUGUGGGGGUUGGGGUGGGAUA | 22 | 2729 |
| CCR5-3321 | − | UGUGUGGGGGUUGGGGUGGGAUA | 23 | 2730 |
| CCR5-3322 | − | CUGUGUGGGGGUUGGGGUGGGAUA | 24 | 2731 |
| CCR5-3323 | − | CAGGGUUAAUGUGAAGUC | 18 | 2732 |
| CCR5-3324 | − | ACAGGGUUAAUGUGAAGUC | 19 | 2733 |
| CCR5-3325 | − | CACAGGGUUAAUGUGAAGUC | 20 | 2734 |
| CCR5-3326 | − | GUACAAAUCAUUUGCUUC | 18 | 2735 |
| CCR5-3327 | − | UGUACAAAUCAUUUGCUUC | 19 | 2736 |
| CCR5-3328 | − | UUGUACAAAUCAUUUGCUUC | 20 | 2737 |
| CCR5-3329 | − | CUUGUACAAAUCAUUUGCUUC | 21 | 2738 |
| CCR5-3330 | − | UCUUGUACAAAUCAUUUGCUUC | 22 | 2739 |
| CCR5-3331 | − | AUCUUGUACAAAUCAUUUGCUUC | 23 | 2740 |
| CCR5-3332 | − | GAUCUUGUACAAAUCAUUUGCUUC | 24 | 2741 |
| CCR5-3333 | − | AGAAAGAUUUGCAGAGAG | 18 | 2742 |
| CCR5-3334 | − | AAGAAAGAUUUGCAGAGAG | 19 | 2743 |
| CCR5-3335 | − | AAAGAAAGAUUUGCAGAGAG | 20 | 2744 |
| CCR5-3336 | − | AAAAGAAAGAUUUGCAGAGAG | 21 | 2745 |
| CCR5-3337 | − | CAAAAGAAAGAUUUGCAGAGAG | 22 | 2746 |
| CCR5-3338 | − | UCAAAAGAAAGAUUUGCAGAGAG | 23 | 2747 |
| CCR5-3339 | − | CUCAAAAGAAAGAUUUGCAGAGAG | 24 | 2748 |
| CCR5-3340 | − | UGUUAGUUAGCUUCUGAG | 18 | 2749 |
| CCR5-3341 | − | CUGUUAGUUAGCUUCUGAG | 19 | 2750 |
| CCR5-3342 | − | CCUGUUAGUUAGCUUCUGAG | 20 | 2751 |
| CCR5-3343 | − | CUAACAGAUUCUGUGUAG | 18 | 2752 |
| CCR5-3344 | − | UCUAACAGAUUCUGUGUAG | 19 | 2753 |
| CCR5-2950 | − | UUCUAACAGAUUCUGUGUAG | 20 | 2754 |
| CCR5-3345 | − | UGGGAUAGGGGAUACGGG | 18 | 2755 |
| CCR5-3346 | − | GUGGGAUAGGGGAUACGGG | 19 | 2756 |
| CCR5-3347 | − | GGUGGGAUAGGGGAUACGGG | 20 | 2757 |
| CCR5-3348 | − | UCUGUGUGGGGGUUGGGG | 18 | 2758 |
| CCR5-3349 | − | AUCUGUGUGGGGGUUGGGG | 19 | 2759 |
| CCR5-2955 | − | CAUCUGUGUGGGGGUUGGGG | 20 | 2760 |
| CCR5-3350 | − | GCAUCUGUGUGGGGGUUGGGG | 21 | 2761 |
| CCR5-3351 | − | AGCAUCUGUGUGGGGGUUGGGG | 22 | 2762 |
| CCR5-3352 | − | GAGCAUCUGUGUGGGGGUUGGGG | 23 | 2763 |
| CCR5-3353 | − | UGAGCAUCUGUGUGGGGGUUGGGG | 24 | 2764 |
| CCR5-3354 | − | GAGCAUCUGUGUGGGGGU | 18 | 2765 |
| CCR5-3355 | − | UGAGCAUCUGUGUGGGGGU | 19 | 2766 |
| CCR5-2970 | − | GUGAGCAUCUGUGUGGGGGU | 20 | 2767 |
| CCR5-3356 | − | GGUGGUGAGCAUCUGUGU | 18 | 2768 |
| CCR5-3357 | − | GGGUGGUGAGCAUCUGUGU | 19 | 2769 |
| CCR5-2973 | − | UGGGUGGUGAGCAUCUGUGU | 20 | 2770 |
| CCR5-3358 | − | AAGAUACAAAACAUGAUU | 18 | 2771 |
| CCR5-3359 | − | AAAGAUACAAAACAUGAUU | 19 | 2772 |
| CCR5-3360 | − | CAAAGAUACAAAACAUGAUU | 20 | 2773 |
| CCR5-3361 | − | UCUCCAGUGAGAUGCCUU | 18 | 2774 |
| CCR5-3362 | − | CUCUCCAGUGAGAUGCCUU | 19 | 2775 |
| CCR5-3363 | − | CCUCUCCAGUGAGAUGCCUU | 20 | 2776 |
| CCR5-3364 | − | AAGGAAAGGGUCACAGUU | 18 | 2777 |
| CCR5-3365 | − | UAAGGAAAGGGUCACAGUU | 19 | 2778 |
| CCR5-2977 | − | AUAAGGAAAGGGUCACAGUU | 20 | 2779 |
| CCR5-3366 | − | GAUAAGGAAAGGGUCACAGUU | 21 | 2780 |
| CCR5-3367 | − | AGAUAAGGAAAGGGUCACAGUU | 22 | 2781 |
| CCR5-3368 | − | AAGAUAAGGAAAGGGUCACAGUU | 23 | 2782 |
| CCR5-3369 | − | UAAGAUAAGGAAAGGGUCACAGUU | 24 | 2783 |

Table 6C provides exemplary targeting domains for knocking down the CCR5 gene selected according to the third tier parameters. The targeting domains bind within 500 bp (e.g., upstream or downstream) of a transcription start site (TSS) and PAM is NNGRRV. It is contemplated herein that in an embodiment the targeting domain hybridizes to the target domain through complementary base pairing. Any of the targeting domains in the table can be used with a S. aureus eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 6C
3rd Tier

| gRNA | DNA | | Target Site | |
|---|---|---|---|---|
| Name | Strand | Targeting Domain | Length | SEQ ID NO |
| CCR5-4045 | + | GGGCAACAAAAUAGUGAA | 18 | 3483 |
| CCR5-4046 | + | AGGGCAACAAAAUAGUGAA | 19 | 3484 |
| CCR5-4047 | + | AAGGGCAACAAAAUAGUGAA | 20 | 3485 |
| CCR5-4048 | + | GAAGGGCAACAAAAUAGUGAA | 21 | 3486 |
| CCR5-4049 | + | UGAAGGGCAACAAAAUAGUGAA | 22 | 3487 |
| CCR5-4050 | + | UUGAAGGGCAACAAAAUAGUGAA | 23 | 3488 |
| CCR5-4051 | + | UUUGAAGGGCAACAAAAUAGUGAA | 24 | 3489 |
| CCR5-4052 | + | UUUUAAUUUUGAACCAUA | 18 | 3490 |
| CCR5-4053 | + | UUUUUAAUUUUGAACCAUA | 19 | 3491 |
| CCR5-4054 | + | AUUUUUAAUUUUGAACCAUA | 20 | 3492 |
| CCR5-4055 | + | CAUUUUUAAUUUUGAACCAUA | 21 | 3493 |
| CCR5-4056 | + | UCAUUUUUAAUUUUGAACCAUA | 22 | 3494 |
| CCR5-4057 | + | CUCAUUUUUAAUUUUGAACCAUA | 23 | 3495 |
| CCR5-4058 | + | GCUCAUUUUUAAUUUUGAACCAUA | 24 | 3496 |
| CCR5-4059 | + | AAAAUCCCCACUAAGAUC | 18 | 3497 |
| CCR5-4060 | + | GAAAAUCCCCACUAAGAUC | 19 | 3498 |
| CCR5-4061 | + | UGAAAAUCCCCACUAAGAUC | 20 | 3499 |
| CCR5-4062 | + | GUGAAAAUCCCCACUAAGAUC | 21 | 3500 |
| CCR5-4063 | + | AGUGAAAAUCCCCACUAAGAUC | 22 | 3501 |
| CCR5-4064 | + | GAGUGAAAAUCCCCACUAAGAUC | 23 | 3502 |
| CCR5-4065 | + | AGAGUGAAAAUCCCCACUAAGAUC | 24 | 3503 |
| CCR5-4066 | + | CUUCAGAUAGAUUAUAUC | 18 | 3504 |
| CCR5-4067 | + | GCUUCAGAUAGAUUAUAUC | 19 | 3505 |
| CCR5-3092 | + | AGCUUCAGAUAGAUUAUAUC | 20 | 3506 |
| CCR5-4068 | + | UAGCUUCAGAUAGAUUAUAUC | 21 | 3507 |
| CCR5-4069 | + | AUAGCUUCAGAUAGAUUAUAUC | 22 | 3508 |
| CCR5-4070 | + | CAUAGCUUCAGAUAGAUUAUAUC | 23 | 3509 |
| CCR5-4071 | + | UCAUAGCUUCAGAUAGAUUAUAUC | 24 | 3510 |
| CCR5-4072 | + | GAGGGCAUCUUGUGGCUC | 18 | 3511 |
| CCR5-4073 | + | AGAGGGCAUCUUGUGGCUC | 19 | 3512 |
| CCR5-3095 | + | CAGAGGGCAUCUUGUGGCUC | 20 | 3513 |
| CCR5-4074 | + | CCAGAGGGCAUCUUGUGGCUC | 21 | 3514 |
| CCR5-4075 | + | CCCAGAGGGCAUCUUGUGGCUC | 22 | 3515 |
| CCR5-4076 | + | GCCCAGAGGGCAUCUUGUGGCUC | 23 | 3516 |
| CCR5-4077 | + | AGCCCAGAGGGCAUCUUGUGGCUC | 24 | 3517 |
| CCR5-4078 | + | UUUCGUCUGCCACCACAG | 18 | 3518 |
| CCR5-4079 | + | GUUUCGUCUGCCACCACAG | 19 | 3519 |
| CCR5-4080 | + | UGUUUCGUCUGCCACCACAG | 20 | 3520 |
| CCR5-4081 | + | AUGUUUCGUCUGCCACCACAG | 21 | 3521 |
| CCR5-4082 | + | AAUGUUUCGUCUGCCACCACAG | 22 | 3522 |
| CCR5-4083 | + | AAAUGUUUCGUCUGCCACCACAG | 23 | 3523 |
| CCR5-4084 | + | AAAAUGUUUCGUCUGCCACCACAG | 24 | 3524 |
| CCR5-4085 | + | UAGAUUAUAUCUGGAGUG | 18 | 3525 |
| CCR5-4086 | + | AUAGAUUAUAUCUGGAGUG | 19 | 3526 |
| CCR5-4087 | + | GAUAGAUUAUAUCUGGAGUG | 20 | 3527 |
| CCR5-4088 | + | AGAUAGAUUAUAUCUGGAGUG | 21 | 3528 |
| CCR5-4089 | + | CAGAUAGAUUAUAUCUGGAGUG | 22 | 3529 |
| CCR5-4090 | + | UCAGAUAGAUUAUAUCUGGAGUG | 23 | 3530 |
| CCR5-4091 | + | UUCAGAUAGAUUAUAUCUGGAGUG | 24 | 3531 |
| CCR5-4092 | + | UUUCUCUUAUUAAACCCU | 18 | 3532 |
| CCR5-4093 | + | UUUUCUCUUAUUAAACCCU | 19 | 3533 |
| CCR5-4094 | + | AUUUUCUCUUAUUAAACCCU | 20 | 3534 |
| CCR5-4095 | + | AAUUUUCUCUUAUUAAACCCU | 21 | 3535 |
| CCR5-4096 | + | GAAUUUUCUCUUAUUAAACCCU | 22 | 3536 |
| CCR5-4097 | + | AGAAUUUUCUCUUAUUAAACCCU | 23 | 3537 |
| CCR5-4098 | + | GAGAAUUUUCUCUUAUUAAACCCU | 24 | 3538 |
| CCR5-4099 | + | AGUUCAGCUGCUCUAGCU | 18 | 3539 |
| CCR5-4100 | + | AAGUUCAGCUGCUCUAGCU | 19 | 3540 |
| CCR5-4101 | + | UAAGUUCAGCUGCUCUAGCU | 20 | 3541 |
| CCR5-4102 | + | UUAAGUUCAGCUGCUCUAGCU | 21 | 3542 |
| CCR5-4103 | + | UUUAAGUUCAGCUGCUCUAGCU | 22 | 3543 |
| CCR5-4104 | + | AUUUAAGUUCAGCUGCUCUAGCU | 23 | 3544 |
| CCR5-4105 | + | UAUUUAAGUUCAGCUGCUCUAGCU | 24 | 3545 |
| CCR5-4106 | + | CUAUGUAUCUGGCAUAGU | 18 | 3546 |
| CCR5-4107 | + | CCUAUGUAUCUGGCAUAGU | 19 | 3547 |
| CCR5-4108 | + | ACCUAUGUAUCUGGCAUAGU | 20 | 3548 |
| CCR5-4109 | + | CACCUAUGUAUCUGGCAUAGU | 21 | 3549 |
| CCR5-4110 | + | CCACCUAUGUAUCUGGCAUAGU | 22 | 3550 |
| CCR5-4111 | + | GCCACCUAUGUAUCUGGCAUAGU | 23 | 3551 |
| CCR5-4112 | + | UGCCACCUAUGUAUCUGGCAUAGU | 24 | 3552 |
| CCR5-4113 | + | UUCUGAGUUGCCACAAUU | 18 | 3553 |
| CCR5-4114 | + | UUUCUGAGUUGCCACAAUU | 19 | 3554 |
| CCR5-4115 | + | GUUUCUGAGUUGCCACAAUU | 20 | 3555 |
| CCR5-4116 | + | AGUUUCUGAGUUGCCACAAUU | 21 | 3556 |
| CCR5-4117 | + | UAGUUUCUGAGUUGCCACAAUU | 22 | 3557 |
| CCR5-4118 | + | GUAGUUUCUGAGUUGCCACAAUU | 23 | 3558 |
| CCR5-4119 | + | UGUAGUUUCUGAGUUGCCACAAUU | 24 | 3559 |
| CCR5-4120 | + | AGAUGAAUGUCAUGCAUU | 18 | 3560 |
| CCR5-4121 | + | CAGAUGAAUGUCAUGCAUU | 19 | 3561 |
| CCR5-4122 | + | ACAGAUGAAUGUCAUGCAUU | 20 | 3562 |
| CCR5-4123 | + | CACAGAUGAAUGUCAUGCAUU | 21 | 3563 |
| CCR5-4124 | + | CCACAGAUGAAUGUCAUGCAUU | 22 | 3564 |
| CCR5-4125 | + | ACCACAGAUGAAUGUCAUGCAUU | 23 | 3565 |
| CCR5-4126 | + | CACCACAGAUGAAUGUCAUGCAUU | 24 | 3566 |
| CCR5-4127 | + | GCACGUAAUUUUGCUGUU | 18 | 3567 |
| CCR5-4128 | + | GGCACGUAAUUUUGCUGUU | 19 | 3568 |
| CCR5-3141 | + | GGGCACGUAAUUUUGCUGUU | 20 | 3569 |
| CCR5-4129 | + | GGGGCACGUAAUUUUGCUGUU | 21 | 3570 |
| CCR5-4130 | + | GGGGGCACGUAAUUUUGCUGUU | 22 | 3571 |
| CCR5-4131 | + | UGGGGGCACGUAAUUUUGCUGUU | 23 | 3572 |
| CCR5-4132 | + | UUGGGGGCACGUAAUUUUGCUGUU | 24 | 3573 |
| CCR5-4133 | + | AGUUUGUGUUUGUAGUUU | 18 | 3574 |
| CCR5-4134 | + | AAGUUUGUGUUUGUAGUUU | 19 | 3575 |
| CCR5-4135 | + | GAAGUUUGUGUUUGUAGUUU | 20 | 3576 |
| CCR5-4136 | + | UGAAGUUUGUGUUUGUAGUUU | 21 | 3577 |
| CCR5-4137 | + | GUGAAGUUUGUGUUUGUAGUUU | 22 | 3578 |
| CCR5-4138 | + | UGUGAAGUUUGUGUUUGUAGUUU | 23 | 3579 |
| CCR5-4139 | + | CUGUGAAGUUUGUGUUUGUAGUUU | 24 | 3580 |
| CCR5-4140 | − | UGCCUAGUCUAAGGUGCA | 18 | 3581 |
| CCR5-4141 | − | CUGCCUAGUCUAAGGUGCA | 19 | 3582 |
| CCR5-3067 | − | GCUGCCUAGUCUAAGGUGCA | 20 | 3583 |
| CCR5-4142 | − | AGCUGCCUAGUCUAAGGUGCA | 21 | 3584 |
| CCR5-4143 | − | CAGCUGCCUAGUCUAAGGUGCA | 22 | 3585 |
| CCR5-4144 | − | UCAGCUGCCUAGUCUAAGGUGCA | 23 | 3586 |
| CCR5-4145 | − | CUCAGCUGCCUAGUCUAAGGUGCA | 24 | 3587 |
| CCR5-4146 | − | CAGGGAGUUUGAGACUCA | 18 | 3588 |
| CCR5-4147 | − | GCAGGGAGUUUGAGACUCA | 19 | 3589 |
| CCR5-4148 | − | UGCAGGGAGUUUGAGACUCA | 20 | 3590 |
| CCR5-4149 | − | GUGCAGGGAGUUUGAGACUCA | 21 | 3591 |
| CCR5-4150 | − | GGUGCAGGGAGUUUGAGACUCA | 22 | 3592 |
| CCR5-4151 | − | AGGUGCAGGGAGUUUGAGACUCA | 23 | 3593 |
| CCR5-4152 | − | AAGGUGCAGGGAGUUUGAGACUCA | 24 | 3594 |
| CCR5-4153 | − | CCCAUCUUUUUCUGGACC | 18 | 3595 |
| CCR5-4154 | − | UCCCAUCUUUUUCUGGACC | 19 | 3596 |
| CCR5-4155 | − | UUCCCAUCUUUUUCUGGACC | 20 | 3597 |
| CCR5-4156 | − | UUUCCCAUCUUUUUCUGGACC | 21 | 3598 |
| CCR5-4157 | − | GUUUCCCAUCUUUUUCUGGACC | 22 | 3599 |
| CCR5-4158 | − | GGUUUCCCAUCUUUUUCUGGACC | 23 | 3600 |
| CCR5-4159 | − | AGGUUUCCCAUCUUUUUCUGGACC | 24 | 3601 |
| CCR5-4160 | − | UUAUAAGACUAAACUACC | 18 | 3602 |
| CCR5-4161 | − | GUUAUAAGACUAAACUACC | 19 | 3603 |
| CCR5-4162 | − | GGUUAUAAGACUAAACUACC | 20 | 3604 |
| CCR5-4163 | − | UGGUUAUAAGACUAAACUACC | 21 | 3605 |
| CCR5-4164 | − | CUGGUUAUAAGACUAAACUACC | 22 | 3606 |
| CCR5-4165 | − | GCUGGUUAUAAGACUAAACUACC | 23 | 3607 |
| CCR5-4166 | − | AGCUGGUUAUAAGACUAAACUACC | 24 | 3608 |
| CCR5-4167 | − | AGUUUUAACUAUGGGCUC | 18 | 3609 |
| CCR5-4168 | − | GAGUUUUAACUAUGGGCUC | 19 | 3610 |
| CCR5-4169 | − | AGAGUUUUAACUAUGGGCUC | 20 | 3611 |
| CCR5-4170 | − | AAGAGUUUUAACUAUGGGCUC | 21 | 3612 |
| CCR5-4171 | − | AAAGAGUUUUAACUAUGGGCUC | 22 | 3613 |
| CCR5-4172 | − | UAAAGAGUUUUAACUAUGGGCUC | 23 | 3614 |
| CCR5-4173 | − | CUAAAGAGUUUUAACUAUGGGCUC | 24 | 3615 |
| CCR5-4174 | − | CUUCCGUGACCUUGGCUC | 18 | 3616 |
| CCR5-4175 | − | GCUUCCGUGACCUUGGCUC | 19 | 3617 |
| CCR5-4176 | − | GGCUUCCGUGACCUUGGCUC | 20 | 3618 |
| CCR5-4177 | − | GGGCUUCCGUGACCUUGGCUC | 21 | 3619 |
| CCR5-4178 | − | UGGGCUUCCGUGACCUUGGCUC | 22 | 3620 |
| CCR5-4179 | − | CUGGGCUUCCGUGACCUUGGCUC | 23 | 3621 |
| CCR5-4180 | − | UCUGGGCUUCCGUGACCUUGGCUC | 24 | 3622 |
| CCR5-4181 | − | UUUUUAUUAUAUUAUUUC | 18 | 3623 |
| CCR5-4182 | − | UUUUUUAUUAUAUUAUUUC | 19 | 3624 |
| CCR5-4183 | − | AUUUUUUAUUAUAUUAUUUC | 20 | 3625 |
| CCR5-4184 | − | CAUUUUUUAUUAUAUUAUUUC | 21 | 3626 |
| CCR5-4185 | − | ACAUUUUUUAUUAUAUUAUUUC | 22 | 3627 |
| CCR5-4186 | − | AACAUUUUUUAUUAUAUUAUUUC | 23 | 3628 |
| CCR5-4187 | − | AAACAUUUUUUAUUAUAUUAUUUC | 24 | 3629 |
| CCR5-4188 | − | UGCCAGAUACAUAGGUGG | 18 | 3630 |
| CCR5-4189 | − | AUGCCAGAUACAUAGGUGG | 19 | 3631 |
| CCR5-4190 | − | UAUGCCAGAUACAUAGGUGG | 20 | 3632 |
| CCR5-4191 | − | CUAUGCCAGAUACAUAGGUGG | 21 | 3633 |
| CCR5-4192 | − | ACUAUGCCAGAUACAUAGGUGG | 22 | 3634 |
| CCR5-4193 | − | CACUAUGCCAGAUACAUAGGUGG | 23 | 3635 |
| CCR5-4194 | − | ACACUAUGCCAGAUACAUAGGUGG | 24 | 3636 |
| CCR5-4195 | − | UGGACCCAGGAUCUUAGU | 18 | 3637 |
| CCR5-4196 | − | CUGGACCCAGGAUCUUAGU | 19 | 3638 |
| CCR5-3135 | − | UCUGGACCCAGGAUCUUAGU | 20 | 3639 |
| CCR5-4197 | − | UUCUGGACCCAGGAUCUUAGU | 21 | 3640 |
| CCR5-4198 | − | UUUCUGGACCCAGGAUCUUAGU | 22 | 3641 |
| CCR5-4199 | − | UUUUCUGGACCCAGGAUCUUAGU | 23 | 3642 |
| CCR5-4200 | − | UUUUUCUGGACCCAGGAUCUUAGU | 24 | 3643 |
| CCR5-4201 | − | AAACUUCACAGAAAAUGU | 18 | 3644 |
| CCR5-4202 | − | CAAACUUCACAGAAAAUGU | 19 | 3645 |
| CCR5-4203 | − | ACAAACUUCACAGAAAAUGU | 20 | 3646 |
| CCR5-4204 | − | CACAAACUUCACAGAAAAUGU | 21 | 3647 |
| CCR5-4205 | − | ACACAAACUUCACAGAAAAUGU | 22 | 3648 |
| CCR5-4206 | − | AACACAAACUUCACAGAAAAUGU | 23 | 3649 |
| CCR5-4207 | − | AAACACAAACUUCACAGAAAAUGU | 24 | 3650 |

Table 6D provides exemplary targeting domains for knocking down the CCR5 gene selected according to the fourth tier parameters. Within the additional 500 bp (e.g., upstream or downstream) of a transcription start site (TSS), e.g., extending to 1 kb upstream and downstream of a TSS and PAM is NNGRRT. It is contemplated herein that in an embodiment the targeting domain hybridizes to the target domain through complementary base pairing. Any of the targeting domains in the table can be used with a S. aureus eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 6D
4th Tier

| gRNA | DNA | | Target Site | |
|---|---|---|---|---|
| Name | Strand | Targeting Domain | Length | SEQ ID NO |
| CCR5-3370 | + | AAGCCCACAUUUUAGUAA | 18 | 2784 |
| CCR5-3371 | + | AAAGCCCACAUUUUAGUAA | 19 | 2785 |
| CCR5-3372 | + | AAAAGCCCACAUUUUAGUAA | 20 | 2786 |
| CCR5-3373 | + | CAAAAGCCCACAUUUUAGUAA | 21 | 2787 |
| CCR5-3374 | + | UCAAAAGCCCACAUUUUAGUAA | 22 | 2788 |
| CCR5-3375 | + | GUCAAAAGCCCACAUUUUAGUAA | 23 | 2789 |
| CCR5-3376 | + | AGUCAAAAGCCCACAUUUUAGUAA | 24 | 2790 |
| CCR5-3377 | + | UGAAGGCGAAAAGAAUCA | 18 | 2791 |
| CCR5-3378 | + | UUGAAGGCGAAAAGAAUCA | 19 | 2792 |
| CCR5-3379 | + | AUUGAAGGCGAAAAGAAUCA | 20 | 2793 |
| CCR5-3380 | + | UAUUGAAGGCGAAAAGAAUCA | 21 | 2794 |
| CCR5-3381 | + | GUAUUGAAGGCGAAAAGAAUCA | 22 | 2795 |
| CCR5-3382 | + | UGUAUUGAAGGCGAAAAGAAUCA | 23 | 2796 |
| CCR5-3383 | + | GUGUAUUGAAGGCGAAAAGAAUCA | 24 | 2797 |
| CCR5-3384 | + | AUGAUUUGUACAAGAUCA | 18 | 2798 |
| CCR5-3385 | + | AAUGAUUUGUACAAGAUCA | 19 | 2799 |
| CCR5-3386 | + | AAAUGAUUUGUACAAGAUCA | 20 | 2800 |
| CCR5-3387 | + | CAAAUGAUUUGUACAAGAUCA | 21 | 2801 |
| CCR5-3388 | + | GCAAAUGAUUUGUACAAGAUCA | 22 | 2802 |
| CCR5-3389 | + | AGCAAAUGAUUUGUACAAGAUCA | 23 | 2803 |
| CCR5-3390 | + | AAGCAAAUGAUUUGUACAAGAUCA | 24 | 2804 |
| CCR5-3391 | + | UAUUCAGAAGGCAUCUCA | 18 | 2805 |
| CCR5-3392 | + | AUAUUCAGAAGGCAUCUCA | 19 | 2806 |
| CCR5-3393 | + | CAUAUUCAGAAGGCAUCUCA | 20 | 2807 |
| CCR5-3394 | + | ACCAACUUUAAAUGUAGA | 18 | 2808 |
| CCR5-3395 | + | AACCAACUUUAAAUGUAGA | 19 | 2809 |
| CCR5-2918 | + | AAACCAACUUUAAAUGUAGA | 20 | 2810 |
| CCR5-3396 | + | UAAACCAACUUUAAAUGUAGA | 21 | 2811 |
| CCR5-3397 | + | UUAAACCAACUUUAAAUGUAGA | 22 | 2812 |
| CCR5-3398 | + | CUUAAACCAACUUUAAAUGUAGA | 23 | 2813 |
| CCR5-3399 | + | ACUUAAACCAACUUUAAAUGUAGA | 24 | 2814 |
| CCR5-3400 | + | AAAUGCUGUUUCUUUUGA | 18 | 2815 |
| CCR5-3401 | + | GAAAUGCUGUUUCUUUUGA | 19 | 2816 |
| CCR5-2921 | + | GGAAAUGCUGUUUCUUUUGA | 20 | 2817 |
| CCR5-3402 | + | AGGAAAUGCUGUUUCUUUUGA | 21 | 2818 |
| CCR5-3403 | + | UAGGAAAUGCUGUUUCUUUUGA | 22 | 2819 |
| CCR5-3404 | + | GUAGGAAAUGCUGUUUCUUUUGA | 23 | 2820 |
| CCR5-3405 | + | AGUAGGAAAUGCUGUUUCUUUUGA | 24 | 2821 |
| CCR5-3406 | + | AAACCAACUUUAAAUGUA | 18 | 2822 |
| CCR5-3407 | + | UAAACCAACUUUAAAUGUA | 19 | 2823 |
| CCR5-3408 | + | UUAAACCAACUUUAAAUGUA | 20 | 2824 |
| CCR5-3409 | + | CUUAAACCAACUUUAAAUGUA | 21 | 2825 |
| CCR5-3410 | + | ACUUAAACCAACUUUAAAUGUA | 22 | 2826 |
| CCR5-3411 | + | AACUUAAACCAACUUUAAAUGUA | 23 | 2827 |
| CCR5-3412 | + | CAACUUAAACCAACUUUAAAUGUA | 24 | 2828 |
| CCR5-3413 | + | GUUAAAUCAUUAAGUGUA | 18 | 2829 |
| CCR5-3414 | + | AGUUAAAUCAUUAAGUGUA | 19 | 2830 |
| CCR5-3415 | + | GAGUUAAAUCAUUAAGUGUA | 20 | 2831 |
| CCR5-3416 | + | GGAGUUAAAUCAUUAAGUGUA | 21 | 2832 |
| CCR5-3417 | + | UGGAGUUAAAUCAUUAAGUGUA | 22 | 2833 |
| CCR5-3418 | + | GUGGAGUUAAAUCAUUAAGUGUA | 23 | 2834 |
| CCR5-3419 | + | GGUGGAGUUAAAUCAUUAAGUGUA | 24 | 2835 |
| CCR5-3420 | + | CGGGGAGAGUUUCUUGUA | 18 | 2836 |
| CCR5-3421 | + | CCGGGGAGAGUUUCUUGUA | 19 | 2837 |
| CCR5-2929 | + | ACCGGGGAGAGUUUCUUGUA | 20 | 2838 |
| CCR5-3422 | + | UACCGGGGAGAGUUUCUUGUA | 21 | 2839 |
| CCR5-3423 | + | UUACCGGGGAGAGUUUCUUGUA | 22 | 2840 |
| CCR5-3424 | + | CUUACCGGGGAGAGUUUCUUGUA | 23 | 2841 |
| CCR5-3425 | + | ACUUACCGGGGAGAGUUUCUUGUA | 24 | 2842 |
| CCR5-3426 | + | CAGCUGAGAGGUUACUUA | 18 | 2843 |
| CCR5-3427 | + | GCAGCUGAGAGGUUACUUA | 19 | 2844 |
| CCR5-3428 | + | AGCAGCUGAGAGGUUACUUA | 20 | 2845 |
| CCR5-3429 | + | AAGCAGCUGAGAGGUUACUUA | 21 | 2846 |
| CCR5-3430 | + | CAAGCAGCUGAGAGGUUACUUA | 22 | 2847 |
| CCR5-3431 | + | CCAAGCAGCUGAGAGGUUACUUA | 23 | 2848 |
| CCR5-3432 | + | GCCAAGCAGCUGAGAGGUUACUUA | 24 | 2849 |
| CCR5-3433 | + | AUUCAGAAGGCAUCUCAC | 18 | 2850 |
| CCR5-3434 | + | UAUUCAGAAGGCAUCUCAC | 19 | 2851 |
| CCR5-2932 | + | AUAUUCAGAAGGCAUCUCAC | 20 | 2852 |
| CCR5-3435 | + | AGCUGAGAGGUUACUUAC | 18 | 2853 |
| CCR5-3436 | + | CAGCUGAGAGGUUACUUAC | 19 | 2854 |
| CCR5-2935 | + | GCAGCUGAGAGGUUACUUAC | 20 | 2855 |
| CCR5-3437 | + | AGCAGCUGAGAGGUUACUUAC | 21 | 2856 |
| CCR5-3438 | + | AAGCAGCUGAGAGGUUACUUAC | 22 | 2857 |
| CCR5-3439 | + | CAAGCAGCUGAGAGGUUACUUAC | 23 | 2858 |
| CCR5-3440 | + | CCAAGCAGCUGAGAGGUUACUUAC | 24 | 2859 |
| CCR5-3441 | + | GCUGAGAGGUUACUUACC | 18 | 2860 |
| CCR5-3442 | + | AGCUGAGAGGUUACUUACC | 19 | 2861 |
| CCR5-2937 | + | CAGCUGAGAGGUUACUUACC | 20 | 2862 |
| CCR5-3443 | + | GCAGCUGAGAGGUUACUUACC | 21 | 2863 |
| CCR5-3444 | + | AGCAGCUGAGAGGUUACUUACC | 22 | 2864 |
| CCR5-3445 | + | AAGCAGCUGAGAGGUUACUUACC | 23 | 2865 |
| CCR5-3446 | + | CAAGCAGCUGAGAGGUUACUUACC | 24 | 2866 |
| CCR5-3447 | + | UAAAAGAAAUUACUAUCC | 18 | 2867 |
| CCR5-3448 | + | GUAAAAGAAAUUACUAUCC | 19 | 2868 |
| CCR5-3449 | + | AGUAAAAGAAAUUACUAUCC | 20 | 2869 |
| CCR5-3450 | + | UAGUAAAAGAAAUUACUAUCC | 21 | 2870 |
| CCR5-3451 | + | UUAGUAAAAGAAAUUACUAUCC | 22 | 2871 |
| CCR5-3452 | + | UUUAGUAAAAGAAAUUACUAUCC | 23 | 2872 |
| CCR5-3453 | + | UUUUAGUAAAAGAAAUUACUAUCC | 24 | 2873 |
| CCR5-3454 | + | GUUGAGCUUAAAAUAAGC | 18 | 2874 |
| CCR5-3455 | + | AGUUGAGCUUAAAAUAAGC | 19 | 2875 |
| CCR5-3456 | + | AAGUUGAGCUUAAAAUAAGC | 20 | 2876 |
| CCR5-3457 | + | UAAGUUGAGCUUAAAAUAAGC | 21 | 2877 |
| CCR5-3458 | + | UUAAGUUGAGCUUAAAAUAAGC | 22 | 2878 |
| CCR5-3459 | + | UUUAAGUUGAGCUUAAAAUAAGC | 23 | 2879 |
| CCR5-3460 | + | UUUUAAGUUGAGCUUAAAAUAAGC | 24 | 2880 |
| CCR5-3461 | + | AAUAAAGGAUAUCAGAGC | 18 | 2881 |
| CCR5-3462 | + | GAAUAAAGGAUAUCAGAGC | 19 | 2882 |
| CCR5-3463 | + | AGAAUAAAGGAUAUCAGAGC | 20 | 2883 |
| CCR5-3464 | + | AAGAAUAAAGGAUAUCAGAGC | 21 | 2884 |
| CCR5-3465 | + | AAAGAAUAAAGGAUAUCAGAGC | 22 | 2885 |
| CCR5-3466 | + | UAAAGAAUAAAGGAUAUCAGAGC | 23 | 2886 |
| CCR5-3467 | + | AUAAAGAAUAAAGGAUAUCAGAGC | 24 | 2887 |
| CCR5-3468 | + | UAAAUGUAGAGGGGGAUC | 18 | 2888 |
| CCR5-3469 | + | UUAAAUGUAGAGGGGGAUC | 19 | 2889 |
| CCR5-3470 | + | UUUAAAUGUAGAGGGGGAUC | 20 | 2890 |
| CCR5-3471 | + | CUUUAAAUGUAGAGGGGGAUC | 21 | 2891 |
| CCR5-3472 | + | ACUUUAAAUGUAGAGGGGGAUC | 22 | 2892 |
| CCR5-3473 | + | AACUUUAAAUGUAGAGGGGGAUC | 23 | 2893 |
| CCR5-3474 | + | CAACUUUAAAUGUAGAGGGGGAUC | 24 | 2894 |
| CCR5-3475 | + | AUAUAGACAGUAUAAAAG | 18 | 2895 |
| CCR5-3476 | + | CAUAUAGACAGUAUAAAAG | 19 | 2896 |
| CCR5-3477 | + | UCAUAUAGACAGUAUAAAAG | 20 | 2897 |
| CCR5-3478 | + | AUCAUAUAGACAGUAUAAAAG | 21 | 2898 |
| CCR5-3479 | + | AAUCAUAUAGACAGUAUAAAAG | 22 | 2899 |
| CCR5-3480 | + | CAAUCAUAUAGACAGUAUAAAAG | 23 | 2900 |
| CCR5-3481 | + | UCAAUCAUAUAGACAGUAUAAAAG | 24 | 2901 |
| CCR5-3482 | + | UCAUUAAGUGUAUUGAAG | 18 | 2902 |
| CCR5-3483 | + | AUCAUUAAGUGUAUUGAAG | 19 | 2903 |
| CCR5-3484 | + | AAUCAUUAAGUGUAUUGAAG | 20 | 2904 |
| CCR5-3485 | + | AAAUCAUUAAGUGUAUUGAAG | 21 | 2905 |
| CCR5-3486 | + | UAAAUCAUUAAGUGUAUUGAAG | 22 | 2906 |
| CCR5-3487 | + | UUAAAUCAUUAAGUGUAUUGAAG | 23 | 2907 |
| CCR5-3488 | + | GUUAAAUCAUUAAGUGUAUUGAAG | 24 | 2908 |
| CCR5-3489 | + | ACAGUUCUUCUUUUUAAG | 18 | 2909 |
| CCR5-3490 | + | AACAGUUCUUCUUUUUAAG | 19 | 2910 |
| CCR5-3491 | + | GAACAGUUCUUCUUUUUAAG | 20 | 2911 |
| CCR5-3492 | + | AGAACAGUUCUUCUUUUUAAG | 21 | 2912 |
| CCR5-3493 | + | GAGAACAGUUCUUCUUUUUAAG | 22 | 2913 |
| CCR5-3494 | + | AGAGAACAGUUCUUCUUUUUAAG | 23 | 2914 |
| CCR5-3495 | + | CAGAGAACAGUUCUUCUUUUUAAG | 24 | 2915 |
| CCR5-3496 | + | CUCAGCUCUUCUGGCCAG | 18 | 2916 |
| CCR5-3497 | + | UCUCAGCUCUUCUGGCCAG | 19 | 2917 |
| CCR5-3498 | + | GUCUCAGCUCUUCUGGCCAG | 20 | 2918 |
| CCR5-3499 | + | UGUCUCAGCUCUUCUGGCCAG | 21 | 2919 |
| CCR5-3500 | + | AUGUCUCAGCUCUUCUGGCCAG | 22 | 2920 |
| CCR5-3501 | + | GAUGUCUCAGCUCUUCUGGCCAG | 23 | 2921 |
| CCR5-3502 | + | GGAUGUCUCAGCUCUUCUGGCCAG | 24 | 2922 |
| CCR5-3503 | + | AACUAACAGGCCAAGCAG | 18 | 2923 |
| CCR5-3504 | + | UAACUAACAGGCCAAGCAG | 19 | 2924 |
| CCR5-3505 | + | CUAACUAACAGGCCAAGCAG | 20 | 2925 |
| CCR5-3506 | + | GCUAACUAACAGGCCAAGCAG | 21 | 2926 |
| CCR5-3507 | + | AGCUAACUAACAGGCCAAGCAG | 22 | 2927 |
| CCR5-3508 | + | AAGCUAACUAACAGGCCAAGCAG | 23 | 2928 |
| CCR5-3509 | + | GAAGCUAACUAACAGGCCAAGCAG | 24 | 2929 |
| CCR5-3510 | + | AAAGGAUAUCAGAGCUAG | 18 | 2930 |
| CCR5-3511 | + | UAAAGGAUAUCAGAGCUAG | 19 | 2931 |
| CCR5-3512 | + | AUAAAGGAUAUCAGAGCUAG | 20 | 2932 |
| CCR5-3513 | + | AAUAAAGGAUAUCAGAGCUAG | 21 | 2933 |
| CCR5-3514 | + | GAAUAAAGGAUAUCAGAGCUAG | 22 | 2934 |
| CCR5-3515 | + | AGAAUAAAGGAUAUCAGAGCUAG | 23 | 2935 |
| CCR5-3516 | + | AAGAAUAAAGGAUAUCAGAGCUAG | 24 | 2936 |
| CCR5-3517 | + | AACCAACUUUAAAUGUAG | 18 | 2937 |
| CCR5-3518 | + | AAACCAACUUUAAAUGUAG | 19 | 2938 |
| CCR5-2949 | + | UAAACCAACUUUAAAUGUAG | 20 | 2939 |
| CCR5-3519 | + | UUAAACCAACUUUAAAUGUAG | 21 | 2940 |
| CCR5-3520 | + | CUUAAACCAACUUUAAAUGUAG | 22 | 2941 |
| CCR5-3521 | + | ACUUAAACCAACUUUAAAUGUAG | 23 | 2942 |
| CCR5-3522 | + | AACUUAAACCAACUUUAAAUGUAG | 24 | 2943 |
| CCR5-3523 | + | GGGGAGAGUUUCUUGUAG | 18 | 2944 |
| CCR5-3524 | + | CGGGGAGAGUUUCUUGUAG | 19 | 2945 |
| CCR5-2820 | + | CCGGGGAGAGUUUCUUGUAG | 20 | 2946 |
| CCR5-3525 | + | ACCGGGGAGAGUUUCUUGUAG | 21 | 2947 |
| CCR5-3526 | + | UACCGGGGAGAGUUUCUUGUAG | 22 | 2948 |
| CCR5-3527 | + | UUACCGGGGAGAGUUUCUUGUAG | 23 | 2949 |
| CCR5-3528 | + | CUUACCGGGGAGAGUUUCUUGUAG | 24 | 2950 |
| CCR5-3529 | + | GGGUUUAGUUCUCCUUAG | 18 | 2951 |
| CCR5-3530 | + | AGGGUUUAGUUCUCCUUAG | 19 | 2952 |
| CCR5-3531 | + | GAGGGUUUAGUUCUCCUUAG | 20 | 2953 |
| CCR5-3532 | + | AGAGGGUUUAGUUCUCCUUAG | 21 | 2954 |
| CCR5-3533 | + | GAGAGGGUUUAGUUCUCCUUAG | 22 | 2955 |
| CCR5-3534 | + | GGAGAGGGUUUAGUUCUCCUUAG | 23 | 2956 |
| CCR5-3535 | + | UGGAGAGGGUUUAGUUCUCCUUAG | 24 | 2957 |
| CCR5-3536 | + | CUGAGAGGUUACUUACCG | 18 | 2958 |
| CCR5-3537 | + | GCUGAGAGGUUACUUACCG | 19 | 2959 |
| CCR5-2821 | + | AGCUGAGAGGUUACUUACCG | 20 | 2960 |
| CCR5-3538 | + | CAGCUGAGAGGUUACUUACCG | 21 | 2961 |
| CCR5-3539 | + | GCAGCUGAGAGGUUACUUACCG | 22 | 2962 |
| CCR5-3540 | + | AGCAGCUGAGAGGUUACUUACCG | 23 | 2963 |
| CCR5-3541 | + | AAGCAGCUGAGAGGUUACUUACCG | 24 | 2964 |
| CCR5-3542 | + | UGUUUCUUUUGAAGGAGG | 18 | 2965 |
| CCR5-3543 | + | CUGUUUCUUUUGAAGGAGG | 19 | 2966 |
| CCR5-3544 | + | GCUGUUUCUUUUGAAGGAGG | 20 | 2967 |
| CCR5-3545 | + | UGCUGUUUCUUUUGAAGGAGG | 21 | 2968 |
| CCR5-3546 | + | AUGCUGUUUCUUUUGAAGGAGG | 22 | 2969 |
| CCR5-3547 | + | AAUGCUGUUUCUUUUGAAGGAGG | 23 | 2970 |
| CCR5-3548 | + | AAAUGCUGUUUCUUUUGAAGGAGG | 24 | 2971 |
| CCR5-3549 | + | UUAAACCAACUUUAAAUG | 18 | 2972 |
| CCR5-3550 | + | CUUAAACCAACUUUAAAUG | 19 | 2973 |
| CCR5-3551 | + | ACUUAAACCAACUUUAAAUG | 20 | 2974 |
| CCR5-3552 | + | AACUUAAACCAACUUUAAAUG | 21 | 2975 |
| CCR5-3553 | + | CAACUUAAACCAACUUUAAAUG | 22 | 2976 |
| CCR5-3554 | + | CCAACUUAAACCAACUUUAAAUG | 23 | 2977 |
| CCR5-3555 | + | GCCAACUUAAACCAACUUUAAAUG | 24 | 2978 |
| CCR5-3556 | + | UCAGAAGGCAUCUCACUG | 18 | 2979 |
| CCR5-3557 | + | UUCAGAAGGCAUCUCACUG | 19 | 2980 |
| CCR5-3558 | + | AUUCAGAAGGCAUCUCACUG | 20 | 2981 |
| CCR5-3559 | + | UAUUCAGAAGGCAUCUCACUG | 21 | 2982 |
| CCR5-3560 | + | AUAUUCAGAAGGCAUCUCACUG | 22 | 2983 |
| CCR5-3561 | + | CAUAUUCAGAAGGCAUCUCACUG | 23 | 2984 |
| CCR5-3562 | + | ACAUAUUCAGAAGGCAUCUCACUG | 24 | 2985 |
| CCR5-3563 | + | ACCGGGGAGAGUUUCUUG | 18 | 2986 |
| CCR5-3564 | + | UACCGGGGAGAGUUUCUUG | 19 | 2987 |
| CCR5-3565 | + | UUACCGGGGAGAGUUUCUUG | 20 | 2988 |
| CCR5-3566 | + | CUUACCGGGGAGAGUUUCUUG | 21 | 2989 |
| CCR5-3567 | + | ACUUACCGGGGAGAGUUUCUUG | 22 | 2990 |
| CCR5-3568 | + | UACUUACCGGGGAGAGUUUCUUG | 23 | 2991 |
| CCR5-3569 | + | UUACUUACCGGGGAGAGUUUCUUG | 24 | 2992 |
| CCR5-3570 | + | GAAAUGCUGUUUCUUUUG | 18 | 2993 |
| CCR5-3571 | + | GGAAAUGCUGUUUCUUUUG | 19 | 2994 |
| CCR5-3572 | + | AGGAAAUGCUGUUUCUUUUG | 20 | 2995 |
| CCR5-3573 | + | UAGGAAAUGCUGUUUCUUUUG | 21 | 2996 |
| CCR5-3574 | + | GUAGGAAAUGCUGUUUCUUUUG | 22 | 2997 |
| CCR5-3575 | + | AGUAGGAAAUGCUGUUUCUUUUG | 23 | 2998 |
| CCR5-3576 | + | AAGUAGGAAAUGCUGUUUCUUUUG | 24 | 2999 |
| CCR5-3577 | + | AUUGAAGGCGAAAAGAAU | 18 | 3000 |
| CCR5-3578 | + | UAUUGAAGGCGAAAAGAAU | 19 | 3001 |
| CCR5-3579 | + | GUAUUGAAGGCGAAAAGAAU | 20 | 3002 |
| CCR5-3580 | + | UGUAUUGAAGGCGAAAAGAAU | 21 | 3003 |
| CCR5-3581 | + | GUGUAUUGAAGGCGAAAAGAAU | 22 | 3004 |
| CCR5-3582 | + | AGUGUAUUGAAGGCGAAAAGAAU | 23 | 3005 |
| CCR5-3583 | + | AAGUGUAUUGAAGGCGAAAAGAAU | 24 | 3006 |
| CCR5-3584 | + | AUAAAGAAUAAAGGAUAU | 18 | 3007 |
| CCR5-3585 | + | UAUAAAGAAUAAAGGAUAU | 19 | 3008 |
| CCR5-3586 | + | AUAUAAAGAAUAAAGGAUAU | 20 | 3009 |
| CCR5-3587 | + | AAUAUAAAGAAUAAAGGAUAU | 21 | 3010 |
| CCR5-3588 | + | AAAUAUAAAGAAUAAAGGAUAU | 22 | 3011 |
| CCR5-3589 | + | AAAAUAUAAAGAAUAAAGGAUAU | 23 | 3012 |
| CCR5-3590 | + | GAAAAUAUAAAGAAUAAAGGAUAU | 24 | 3013 |
| CCR5-3591 | + | CUAACAGGCCAAGCAGCU | 18 | 3014 |
| CCR5-3592 | + | ACUAACAGGCCAAGCAGCU | 19 | 3015 |
| CCR5-3593 | + | AACUAACAGGCCAAGCAGCU | 20 | 3016 |
| CCR5-3594 | + | UAACUAACAGGCCAAGCAGCU | 21 | 3017 |
| CCR5-3595 | + | CUAACUAACAGGCCAAGCAGCU | 22 | 3018 |
| CCR5-3596 | + | GCUAACUAACAGGCCAAGCAGCU | 23 | 3019 |
| CCR5-3597 | + | AGCUAACUAACAGGCCAAGCAGCU | 24 | 3020 |
| CCR5-3598 | + | AAAGUCUUUUACUCAUCU | 18 | 3021 |
| CCR5-3599 | + | UAAAGUCUUUUACUCAUCU | 19 | 3022 |
| CCR5-3600 | + | GUAAAGUCUUUUACUCAUCU | 20 | 3023 |
| CCR5-3601 | + | UGUAAAGUCUUUUACUCAUCU | 21 | 3024 |
| CCR5-3602 | + | CUGUAAAGUCUUUUACUCAUCU | 22 | 3025 |
| CCR5-3603 | + | CCUGUAAAGUCUUUUACUCAUCU | 23 | 3026 |
| CCR5-3604 | + | UCCUGUAAAGUCUUUUACUCAUCU | 24 | 3027 |
| CCR5-3605 | + | UAUAGACAGUAUAAAAGU | 18 | 3028 |
| CCR5-3606 | + | AUAUAGACAGUAUAAAAGU | 19 | 3029 |
| CCR5-2967 | + | CAUAUAGACAGUAUAAAAGU | 20 | 3030 |
| CCR5-3607 | + | UCAUAUAGACAGUAUAAAAGU | 21 | 3031 |
| CCR5-3608 | + | AUCAUAUAGACAGUAUAAAAGU | 22 | 3032 |
| CCR5-3609 | + | AAUCAUAUAGACAGUAUAAAAGU | 23 | 3033 |
| CCR5-3610 | + | CAAUCAUAUAGACAGUAUAAAAGU | 24 | 3034 |
| CCR5-3611 | + | CUUUGAUGUUAUAACCGU | 18 | 3035 |
| CCR5-3612 | + | UCUUUGAUGUUAUAACCGU | 19 | 3036 |
| CCR5-3613 | + | AUCUUUGAUGUUAUAACCGU | 20 | 3037 |
| CCR5-3614 | + | UAUCUUUGAUGUUAUAACCGU | 21 | 3038 |
| CCR5-3615 | + | GUAUCUUUGAUGUUAUAACCGU | 22 | 3039 |
| CCR5-3616 | + | UGUAUCUUUGAUGUUAUAACCGU | 23 | 3040 |
| CCR5-3617 | + | UUGUAUCUUUGAUGUUAUAACCGU | 24 | 3041 |
| CCR5-3618 | + | AGAGAAUAGAUCUCUGGU | 18 | 3042 |
| CCR5-3619 | + | UAGAGAAUAGAUCUCUGGU | 19 | 3043 |
| CCR5-3620 | + | CUAGAGAAUAGAUCUCUGGU | 20 | 3044 |
| CCR5-3621 | + | GCUAGAGAAUAGAUCUCUGGU | 21 | 3045 |
| CCR5-3622 | + | AGCUAGAGAAUAGAUCUCUGGU | 22 | 3046 |
| CCR5-3623 | + | AAGCUAGAGAAUAGAUCUCUGGU | 23 | 3047 |
| CCR5-3624 | + | UAAGCUAGAGAAUAGAUCUCUGGU | 24 | 3048 |
| CCR5-3625 | + | CCACUACACAGAAUCUGU | 18 | 3049 |
| CCR5-3626 | + | CCCACUACACAGAAUCUGU | 19 | 3050 |
| CCR5-3627 | + | UCCCACUACACAGAAUCUGU | 20 | 3051 |
| CCR5-3628 | + | AUCCCACUACACAGAAUCUGU | 21 | 3052 |
| CCR5-3629 | + | CAUCCCACUACACAGAAUCUGU | 22 | 3053 |
| CCR5-3630 | + | UCAUCCCACUACACAGAAUCUGU | 23 | 3054 |
| CCR5-3631 | + | CUCAUCCCACUACACAGAAUCUGU | 24 | 3055 |
| CCR5-3632 | + | AUAUUUUAAGAUAAUUGU | 18 | 3056 |
| CCR5-3633 | + | UAUAUUUUAAGAUAAUUGU | 19 | 3057 |
| CCR5-3634 | + | UUAUAUUUUAAGAUAAUUGU | 20 | 3058 |
| CCR5-3635 | + | AUUAUAUUUUAAGAUAAUUGU | 21 | 3059 |
| CCR5-3636 | + | GAUUAUAUUUUAAGAUAAUUGU | 22 | 3060 |
| CCR5-3637 | + | AGAUUAUAUUUUAAGAUAAUUGU | 23 | 3061 |
| CCR5-3638 | + | AAGAUUAUAUUUUAAGAUAAUUGU | 24 | 3062 |
| CCR5-3639 | + | CCGGGGAGAGUUUCUUGU | 18 | 3063 |
| CCR5-3640 | + | ACCGGGGAGAGUUUCUUGU | 19 | 3064 |
| CCR5-2974 | + | UACCGGGGAGAGUUUCUUGU | 20 | 3065 |
| CCR5-3641 | + | UUACCGGGGAGAGUUUCUUGU | 21 | 3066 |
| CCR5-3642 | + | CUUACCGGGGAGAGUUUCUUGU | 22 | 3067 |
| CCR5-3643 | + | ACUUACCGGGGAGAGUUUCUUGU | 23 | 3068 |
| CCR5-3644 | + | UACUUACCGGGGAGAGUUUCUUGU | 24 | 3069 |
| CCR5-3645 | + | UCUCUGCAAAUCUUUCUU | 18 | 3070 |
| CCR5-3646 | + | CUCUCUGCAAAUCUUUCUU | 19 | 3071 |
| CCR5-3647 | + | UCUCUCUGCAAAUCUUUCUU | 20 | 3072 |
| CCR5-3648 | + | AUCUCUCUGCAAAUCUUUCUU | 21 | 3073 |
| CCR5-3649 | + | CAUCUCUCUGCAAAUCUUUCUU | 22 | 3074 |
| CCR5-3650 | + | UCAUCUCUCUGCAAAUCUUUCUU | 23 | 3075 |
| CCR5-3651 | + | CUCAUCUCUCUGCAAAUCUUUCUU | 24 | 3076 |
| CCR5-3652 | + | UAGGAAAUGCUGUUUCUU | 18 | 3077 |
| CCR5-3653 | + | GUAGGAAAUGCUGUUUCUU | 19 | 3078 |
| CCR5-3654 | + | AGUAGGAAAUGCUGUUUCUU | 20 | 3079 |
| CCR5-3655 | + | AAGUAGGAAAUGCUGUUUCUU | 21 | 3080 |
| CCR5-3656 | + | AAAGUAGGAAAUGCUGUUUCUU | 22 | 3081 |
| CCR5-3657 | + | AAAAGUAGGAAAUGCUGUUUCUU | 23 | 3082 |
| CCR5-3658 | + | UAAAAGUAGGAAAUGCUGUUUCUU | 24 | 3083 |
| CCR5-3659 | + | CAGUAAGGCUAAAAGGUU | 18 | 3084 |
| CCR5-3660 | + | ACAGUAAGGCUAAAAGGUU | 19 | 3085 |
| CCR5-3661 | + | AACAGUAAGGCUAAAAGGUU | 20 | 3086 |
| CCR5-3662 | + | CAACAGUAAGGCUAAAAGGUU | 21 | 3087 |
| CCR5-3663 | + | UCAACAGUAAGGCUAAAAGGUU | 22 | 3088 |
| CCR5-3664 | + | UUCAACAGUAAGGCUAAAAGGUU | 23 | 3089 |
| CCR5-3665 | + | UUUCAACAGUAAGGCUAAAAGGUU | 24 | 3090 |
| CCR5-3666 | + | UGGUCUGAAGGUUUAUUU | 18 | 3091 |
| CCR5-3667 | + | CUGGUCUGAAGGUUUAUUU | 19 | 3092 |
| CCR5-3668 | + | UCUGGUCUGAAGGUUUAUUU | 20 | 3093 |
| CCR5-3669 | + | CUCUGGUCUGAAGGUUUAUUU | 21 | 3094 |
| CCR5-3670 | + | UCUCUGGUCUGAAGGUUUAUUU | 22 | 3095 |
| CCR5-3671 | + | AUCUCUGGUCUGAAGGUUUAUUU | 23 | 3096 |
| CCR5-3672 | + | GAUCUCUGGUCUGAAGGUUUAUUU | 24 | 3097 |
| CCR5-3673 | + | UCUGCAAAUCUUUCUUUU | 18 | 3098 |
| CCR5-3674 | + | CUCUGCAAAUCUUUCUUUU | 19 | 3099 |
| CCR5-3675 | + | UCUCUGCAAAUCUUUCUUUU | 20 | 3100 |
| CCR5-3676 | + | CUCUCUGCAAAUCUUUCUUUU | 21 | 3101 |
| CCR5-3677 | + | UCUCUCUGCAAAUCUUUCUUUU | 22 | 3102 |
| CCR5-3678 | + | AUCUCUCUGCAAAUCUUUCUUUU | 23 | 3103 |
| CCR5-3679 | + | CAUCUCUCUGCAAAUCUUUCUUUU | 24 | 3104 |
| CCR5-3680 | − | GGGGAGAGUGGAGAAAAA | 18 | 3105 |
| CCR5-3681 | − | CGGGGAGAGUGGAGAAAAA | 19 | 3106 |
| CCR5-2905 | − | ACGGGGAGAGUGGAGAAAAA | 20 | 3107 |
| CCR5-3682 | − | UACGGGGAGAGUGGAGAAAAA | 21 | 3108 |
| CCR5-3683 | − | AUACGGGGAGAGUGGAGAAAAA | 22 | 3109 |
| CCR5-3684 | − | GAUACGGGGAGAGUGGAGAAAAA | 23 | 3110 |
| CCR5-3685 | − | GGAUACGGGGAGAGUGGAGAAAAA | 24 | 3111 |
| CCR5-3686 | − | CGGGGAGAGUGGAGAAAA | 18 | 3112 |
| CCR5-3687 | − | ACGGGGAGAGUGGAGAAAA | 19 | 3113 |
| CCR5-2906 | − | UACGGGGAGAGUGGAGAAAA | 20 | 3114 |
| CCR5-3688 | − | AUACGGGGAGAGUGGAGAAAA | 21 | 3115 |
| CCR5-3689 | − | GAUACGGGGAGAGUGGAGAAAA | 22 | 3116 |
| CCR5-3690 | − | GGAUACGGGGAGAGUGGAGAAAA | 23 | 3117 |
| CCR5-3691 | − | GGGAUACGGGGAGAGUGGAGAAAA | 24 | 3118 |
| CCR5-3692 | − | ACGGGGAGAGUGGAGAAA | 18 | 3119 |
| CCR5-3693 | − | UACGGGGAGAGUGGAGAAA | 19 | 3120 |
| CCR5-3694 | − | AUACGGGGAGAGUGGAGAAA | 20 | 3121 |
| CCR5-3695 | − | GAUACGGGGAGAGUGGAGAAA | 21 | 3122 |
| CCR5-3696 | − | GGAUACGGGGAGAGUGGAGAAA | 22 | 3123 |
| CCR5-3697 | − | GGGAUACGGGGAGAGUGGAGAAA | 23 | 3124 |
| CCR5-3698 | − | GGGGAUACGGGGAGAGUGGAGAAA | 24 | 3125 |
| CCR5-3699 | − | UUUUAAGCUCAACUUAAA | 18 | 3126 |
| CCR5-3700 | − | AUUUUAAGCUCAACUUAAA | 19 | 3127 |
| CCR5-3701 | − | UAUUUUAAGCUCAACUUAAA | 20 | 3128 |
| CCR5-3702 | − | UUAUUUUAAGCUCAACUUAAA | 21 | 3129 |
| CCR5-3703 | − | CUUAUUUUAAGCUCAACUUAAA | 22 | 3130 |
| CCR5-3704 | − | GCUUAUUUUAAGCUCAACUUAAA | 23 | 3131 |
| CCR5-3705 | − | AGCUUAUUUUAAGCUCAACUUAAA | 24 | 3132 |
| CCR5-3706 | − | UGAGUGAAAGACUUUAAA | 18 | 3133 |
| CCR5-3707 | − | GUGAGUGAAAGACUUUAAA | 19 | 3134 |
| CCR5-2909 | − | UGUGAGUGAAAGACUUUAAA | 20 | 3135 |
| CCR5-3708 | − | UUGUGAGUGAAAGACUUUAAA | 21 | 3136 |
| CCR5-3709 | − | AUUGUGAGUGAAAGACUUUAAA | 22 | 3137 |
| CCR5-3710 | − | GAUUGUGAGUGAAAGACUUUAAA | 23 | 3138 |
| CCR5-3711 | − | UGAUUGUGAGUGAAAGACUUUAAA | 24 | 3139 |
| CCR5-3712 | − | ACAAUCCUUACCUCUCAA | 18 | 3140 |
| CCR5-3713 | − | AACAAUCCUUACCUCUCAA | 19 | 3141 |
| CCR5-3714 | − | UAACAAUCCUUACCUCUCAA | 20 | 3142 |
| CCR5-3715 | − | CUAACAAUCCUUACCUCUCAA | 21 | 3143 |
| CCR5-3716 | − | ACUAACAAUCCUUACCUCUCAA | 22 | 3144 |
| CCR5-3717 | − | AACUAACAAUCCUUACCUCUCAA | 23 | 3145 |
| CCR5-3718 | − | UAACUAACAAUCCUUACCUCUCAA | 24 | 3146 |
| CCR5-3719 | − | AACUCCACCCUCCUUCAA | 18 | 3147 |
| CCR5-3720 | − | UAACUCCACCCUCCUUCAA | 19 | 3148 |
| CCR5-3721 | − | UUAACUCCACCCUCCUUCAA | 20 | 3149 |
| CCR5-3722 | − | UUUAACUCCACCCUCCUUCAA | 21 | 3150 |
| CCR5-3723 | − | AUUUAACUCCACCCUCCUUCAA | 22 | 3151 |
| CCR5-3724 | − | GAUUUAACUCCACCCUCCUUCAA | 23 | 3152 |
| CCR5-3725 | − | UGAUUUAACUCCACCCUCCUUCAA | 24 | 3153 |
| CCR5-3726 | − | GUGAGUGAAAGACUUUAA | 18 | 3154 |
| CCR5-3727 | − | UGUGAGUGAAAGACUUUAA | 19 | 3155 |
| CCR5-2913 | − | UUGUGAGUGAAAGACUUUAA | 20 | 3156 |
| CCR5-3728 | − | AUUGUGAGUGAAAGACUUUAA | 21 | 3157 |
| CCR5-3729 | − | GAUUGUGAGUGAAAGACUUUAA | 22 | 3158 |
| CCR5-3730 | − | UGAUUGUGAGUGAAAGACUUUAA | 23 | 3159 |
| CCR5-3731 | − | AUGAUUGUGAGUGAAAGACUUUAA | 24 | 3160 |
| CCR5-3732 | − | GACUUUACAGGAAACCCA | 18 | 3161 |
| CCR5-3733 | − | AGACUUUACAGGAAACCCA | 19 | 3162 |
| CCR5-3734 | − | AAGACUUUACAGGAAACCCA | 20 | 3163 |
| CCR5-3735 | − | AAAGACUUUACAGGAAACCCA | 21 | 3164 |
| CCR5-3736 | − | AAAAGACUUUACAGGAAACCCA | 22 | 3165 |
| CCR5-3737 | − | UAAAAGACUUUACAGGAAACCCA | 23 | 3166 |
| CCR5-3738 | − | GUAAAAGACUUUACAGGAAACCCA | 24 | 3167 |
| CCR5-3739 | − | CAAAAACAAAAUAAUCCA | 18 | 3168 |
| CCR5-3740 | − | ACAAAAACAAAAUAAUCCA | 19 | 3169 |
| CCR5-3741 | − | AACAAAAACAAAAUAAUCCA | 20 | 3170 |
| CCR5-3742 | − | GAACAAAAACAAAAUAAUCCA | 21 | 3171 |
| CCR5-3743 | − | AGAACAAAAACAAAAUAAUCCA | 22 | 3172 |
| CCR5-3744 | − | GAGAACAAAAACAAAAUAAUCCA | 23 | 3173 |
| CCR5-3745 | − | AGAGAACAAAAACAAAAUAAUCCA | 24 | 3174 |
| CCR5-3746 | − | AGAACUAAACCCUCUCCA | 18 | 3175 |
| CCR5-3747 | − | GAGAACUAAACCCUCUCCA | 19 | 3176 |
| CCR5-3748 | − | GGAGAACUAAACCCUCUCCA | 20 | 3177 |
| CCR5-3749 | − | AGGAGAACUAAACCCUCUCCA | 21 | 3178 |
| CCR5-3750 | − | AAGGAGAACUAAACCCUCUCCA | 22 | 3179 |
| CCR5-3751 | − | UAAGGAGAACUAAACCCUCUCCA | 23 | 3180 |
| CCR5-3752 | − | CUAAGGAGAACUAAACCCUCUCCA | 24 | 3181 |
| CCR5-3753 | − | UGUGUAGUGGGAUGAGCA | 18 | 3182 |
| CCR5-3754 | − | CUGUGUAGUGGGAUGAGCA | 19 | 3183 |
| CCR5-3755 | − | UCUGUGUAGUGGGAUGAGCA | 20 | 3184 |
| CCR5-3756 | − | UUCUGUGUAGUGGGAUGAGCA | 21 | 3185 |
| CCR5-3757 | − | AUUCUGUGUAGUGGGAUGAGCA | 22 | 3186 |
| CCR5-3758 | − | GAUUCUGUGUAGUGGGAUGAGCA | 23 | 3187 |
| CCR5-3759 | − | AGAUUCUGUGUAGUGGGAUGAGCA | 24 | 3188 |
| CCR5-3760 | − | UCAAAAGAAAGAUUUGCA | 18 | 3189 |
| CCR5-3761 | − | CUCAAAAGAAAGAUUUGCA | 19 | 3190 |
| CCR5-3762 | − | UCUCAAAAGAAAGAUUUGCA | 20 | 3191 |
| CCR5-3763 | − | CUCUCAAAAGAAAGAUUUGCA | 21 | 3192 |
| CCR5-3764 | − | CCUCUCAAAAGAAAGAUUUGCA | 22 | 3193 |
| CCR5-3765 | − | ACCUCUCAAAAGAAAGAUUUGCA | 23 | 3194 |
| CCR5-3766 | − | UACCUCUCAAAAGAAAGAUUUGCA | 24 | 3195 |
| CCR5-3767 | − | AUAGGGGAUACGGGGAGA | 18 | 3196 |
| CCR5-3768 | − | GAUAGGGGAUACGGGGAGA | 19 | 3197 |
| CCR5-3769 | − | GGAUAGGGGAUACGGGGAGA | 20 | 3198 |
| CCR5-3770 | − | GGGAUAGGGGAUACGGGGAGA | 21 | 3199 |
| CCR5-3771 | − | UGGGAUAGGGGAUACGGGGAGA | 22 | 3200 |
| CCR5-3772 | − | GUGGGAUAGGGGAUACGGGGAGA | 23 | 3201 |
| CCR5-3773 | − | GGUGGGAUAGGGGAUACGGGGAGA | 24 | 3202 |
| CCR5-3774 | − | GUGGGGGUUGGGGUGGGA | 18 | 3203 |
| CCR5-3775 | − | UGUGGGGGUUGGGGUGGGA | 19 | 3204 |
| CCR5-3776 | − | GUGUGGGGGUUGGGGUGGGA | 20 | 3205 |
| CCR5-3777 | − | UGUGUGGGGGUUGGGGUGGGA | 21 | 3206 |
| CCR5-3778 | − | CUGUGUGGGGGUUGGGGUGGGA | 22 | 3207 |
| CCR5-3779 | − | UCUGUGUGGGGGUUGGGGUGGGA | 23 | 3208 |
| CCR5-3780 | − | AUCUGUGUGGGGGUUGGGGUGGGA | 24 | 3209 |
| CCR5-3781 | − | UACAAAACAUGAUUGUGA | 18 | 3210 |
| CCR5-3782 | − | AUACAAAACAUGAUUGUGA | 19 | 3211 |
| CCR5-3783 | − | GAUACAAAACAUGAUUGUGA | 20 | 3212 |
| CCR5-3784 | − | AGAUACAAAACAUGAUUGUGA | 21 | 3213 |
| CCR5-3785 | − | AAGAUACAAAACAUGAUUGUGA | 22 | 3214 |
| CCR5-3786 | − | AAAGAUACAAAACAUGAUUGUGA | 23 | 3215 |
| CCR5-3787 | − | CAAAGAUACAAAACAUGAUUGUGA | 24 | 3216 |
| CCR5-3788 | − | AAUAUAAUCUUUAAGAUA | 18 | 3217 |
| CCR5-3789 | − | AAAUAUAAUCUUUAAGAUA | 19 | 3218 |
| CCR5-2922 | − | AAAAUAUAAUCUUUAAGAUA | 20 | 3219 |
| CCR5-3790 | − | UAAAAUAUAAUCUUUAAGAUA | 21 | 3220 |
| CCR5-3791 | − | UUAAAAUAUAAUCUUUAAGAUA | 22 | 3221 |
| CCR5-3792 | − | CUUAAAAUAUAAUCUUUAAGAUA | 23 | 3222 |
| CCR5-3793 | − | UCUUAAAAUAUAAUCUUUAAGAUA | 24 | 3223 |
| CCR5-3794 | − | GGGGUGGGAUAGGGGAUA | 18 | 3224 |
| CCR5-3795 | − | UGGGGUGGGAUAGGGGAUA | 19 | 3225 |
| CCR5-2923 | − | UUGGGGUGGGAUAGGGGAUA | 20 | 3226 |
| CCR5-3796 | − | GUUGGGGUGGGAUAGGGGAUA | 21 | 3227 |
| CCR5-3797 | − | GGUUGGGGUGGGAUAGGGGAUA | 22 | 3228 |
| CCR5-3798 | − | GGGUUGGGGUGGGAUAGGGGAUA | 23 | 3229 |
| CCR5-3799 | − | GGGGUUGGGGUGGGAUAGGGGAUA | 24 | 3230 |
| CCR5-3800 | − | AAAUCUUAUCUUCUGCUA | 18 | 3231 |
| CCR5-3801 | − | GAAAUCUUAUCUUCUGCUA | 19 | 3232 |
| CCR5-2925 | − | UGAAAUCUUAUCUUCUGCUA | 20 | 3233 |
| CCR5-3802 | − | UUGAAAUCUUAUCUUCUGCUA | 21 | 3234 |
| CCR5-3803 | − | CUUGAAAUCUUAUCUUCUGCUA | 22 | 3235 |
| CCR5-3804 | − | UCUUGAAAUCUUAUCUUCUGCUA | 23 | 3236 |
| CCR5-3805 | − | AUCUUGAAAUCUUAUCUUCUGCUA | 24 | 3237 |
| CCR5-3806 | − | UCUAACAGAUUCUGUGUA | 18 | 3238 |
| CCR5-3807 | − | UUCUAACAGAUUCUGUGUA | 19 | 3239 |
| CCR5-3808 | − | UUUCUAACAGAUUCUGUGUA | 20 | 3240 |
| CCR5-3809 | − | UUUUCUAACAGAUUCUGUGUA | 21 | 3241 |
| CCR5-3810 | − | AUUUUCUAACAGAUUCUGUGUA | 22 | 3242 |
| CCR5-3811 | − | UAUUUUCUAACAGAUUCUGUGUA | 23 | 3243 |
| CCR5-3812 | − | AUAUUUUCUAACAGAUUCUGUGUA | 24 | 3244 |
| CCR5-3813 | − | GAUGAGUAAAAGACUUUA | 18 | 3245 |
| CCR5-3814 | − | AGAUGAGUAAAAGACUUUA | 19 | 3246 |
| CCR5-3815 | − | GAGAUGAGUAAAAGACUUUA | 20 | 3247 |
| CCR5-3816 | − | UGAGAUGAGUAAAAGACUUUA | 21 | 3248 |
| CCR5-3817 | − | CUGAGAUGAGUAAAAGACUUUA | 22 | 3249 |
| CCR5-3818 | − | UCUGAGAUGAGUAAAAGACUUUA | 23 | 3250 |
| CCR5-3819 | − | UUCUGAGAUGAGUAAAAGACUUUA | 24 | 3251 |
| CCR5-3820 | − | UGUGAGUGAAAGACUUUA | 18 | 3252 |
| CCR5-3821 | − | UUGUGAGUGAAAGACUUUA | 19 | 3253 |
| CCR5-3822 | − | AUUGUGAGUGAAAGACUUUA | 20 | 3254 |
| CCR5-3823 | − | GAUUGUGAGUGAAAGACUUUA | 21 | 3255 |
| CCR5-3824 | − | UGAUUGUGAGUGAAAGACUUUA | 22 | 3256 |
| CCR5-3825 | − | AUGAUUGUGAGUGAAAGACUUUA | 23 | 3257 |
| CCR5-3826 | − | CAUGAUUGUGAGUGAAAGACUUUA | 24 | 3258 |
| CCR5-3827 | − | GUAAAUAAACCUUCAGAC | 18 | 3259 |
| CCR5-3828 | − | CGUAAAUAAACCUUCAGAC | 19 | 3260 |
| CCR5-3829 | − | CCGUAAAUAAACCUUCAGAC | 20 | 3261 |
| CCR5-3830 | − | CCCGUAAAUAAACCUUCAGAC | 21 | 3262 |
| CCR5-3831 | − | GCCCGUAAAUAAACCUUCAGAC | 22 | 3263 |
| CCR5-3832 | − | AGCCCGUAAAUAAACCUUCAGAC | 23 | 3264 |
| CCR5-3833 | − | AAGCCCGUAAAUAAACCUUCAGAC | 24 | 3265 |
| CCR5-3834 | − | GGGUGGGAUAGGGGAUAC | 18 | 3266 |
| CCR5-3835 | − | GGGGUGGGAUAGGGGAUAC | 19 | 3267 |
| CCR5-2934 | − | UGGGGUGGGAUAGGGGAUAC | 20 | 3268 |
| CCR5-3836 | − | UUGGGGUGGGAUAGGGGAUAC | 21 | 3269 |
| CCR5-3837 | − | GUUGGGGUGGGAUAGGGGAUAC | 22 | 3270 |
| CCR5-3838 | − | GGUUGGGGUGGGAUAGGGGAUAC | 23 | 3271 |
| CCR5-3839 | − | GGGUUGGGGUGGGAUAGGGGAUAC | 24 | 3272 |
| CCR5-3840 | − | AGACAUCCGUUCCCCUAC | 18 | 3273 |
| CCR5-3841 | − | GAGACAUCCGUUCCCCUAC | 19 | 3274 |
| CCR5-3842 | − | UGAGACAUCCGUUCCCCUAC | 20 | 3275 |
| CCR5-3843 | − | CUGAGACAUCCGUUCCCCUAC | 21 | 3276 |
| CCR5-3844 | − | GCUGAGACAUCCGUUCCCCUAC | 22 | 3277 |
| CCR5-3845 | − | AGCUGAGACAUCCGUUCCCCUAC | 23 | 3278 |
| CCR5-3846 | − | GAGCUGAGACAUCCGUUCCCCUAC | 24 | 3279 |
| CCR5-3847 | − | AUGAGUAAAAGACUUUAC | 18 | 3280 |
| CCR5-3848 | − | GAUGAGUAAAAGACUUUAC | 19 | 3281 |
| CCR5-2936 | − | AGAUGAGUAAAAGACUUUAC | 20 | 3282 |
| CCR5-3849 | − | GAGAUGAGUAAAAGACUUUAC | 21 | 3283 |
| CCR5-3850 | − | UGAGAUGAGUAAAAGACUUUAC | 22 | 3284 |
| CCR5-3851 | − | CUGAGAUGAGUAAAAGACUUUAC | 23 | 3285 |
| CCR5-3852 | − | UCUGAGAUGAGUAAAAGACUUUAC | 24 | 3286 |
| CCR5-3853 | − | UUGCACAGCUCAUCUGGC | 18 | 3287 |
| CCR5-3854 | − | UUUGCACAGCUCAUCUGGC | 19 | 3288 |
| CCR5-3855 | − | AUUUGCACAGCUCAUCUGGC | 20 | 3289 |
| CCR5-3856 | − | GAUUUGCACAGCUCAUCUGGC | 21 | 3290 |
| CCR5-3857 | − | UGAUUUGCACAGCUCAUCUGGC | 22 | 3291 |
| CCR5-3858 | − | UUGAUUUGCACAGCUCAUCUGGC | 23 | 3292 |
| CCR5-3859 | − | AUUGAUUUGCACAGCUCAUCUGGC | 24 | 3293 |
| CCR5-3860 | − | UGAGUCUUAGCUGAAAUC | 18 | 3294 |
| CCR5-3861 | − | AUGAGUCUUAGCUGAAAUC | 19 | 3295 |
| CCR5-3862 | − | GAUGAGUCUUAGCUGAAAUC | 20 | 3296 |
| CCR5-3863 | − | AGAUGAGUCUUAGCUGAAAUC | 21 | 3297 |
| CCR5-3864 | − | GAGAUGAGUCUUAGCUGAAAUC | 22 | 3298 |
| CCR5-3865 | − | AGAGAUGAGUCUUAGCUGAAAUC | 23 | 3299 |
| CCR5-3866 | − | GAGAGAUGAGUCUUAGCUGAAAUC | 24 | 3300 |
| CCR5-3867 | − | UAAGCUCAACUUAAAAAG | 18 | 3301 |
| CCR5-3868 | − | UUAAGCUCAACUUAAAAAG | 19 | 3302 |
| CCR5-3869 | − | UUUAAGCUCAACUUAAAAAG | 20 | 3303 |
| CCR5-3870 | − | UUUUAAGCUCAACUUAAAAAG | 21 | 3304 |
| CCR5-3871 | − | AUUUUAAGCUCAACUUAAAAAG | 22 | 3305 |
| CCR5-3872 | − | UAUUUUAAGCUCAACUUAAAAAG | 23 | 3306 |
| CCR5-3873 | − | UUAUUUUAAGCUCAACUUAAAAAG | 24 | 3307 |
| CCR5-3874 | − | AUCUUAUCUUCUGCUAAG | 18 | 3308 |
| CCR5-3875 | − | AAUCUUAUCUUCUGCUAAG | 19 | 3309 |
| CCR5-3876 | − | AAAUCUUAUCUUCUGCUAAG | 20 | 3310 |
| CCR5-3877 | − | GAAAUCUUAUCUUCUGCUAAG | 21 | 3311 |
| CCR5-3878 | − | UGAAAUCUUAUCUUCUGCUAAG | 22 | 3312 |
| CCR5-3879 | − | UUGAAAUCUUAUCUUCUGCUAAG | 23 | 3313 |
| CCR5-3880 | − | CUUGAAAUCUUAUCUUCUGCUAAG | 24 | 3314 |
| CCR5-3881 | − | CACAGCUCAUCUGGCCAG | 18 | 3315 |
| CCR5-3882 | − | GCACAGCUCAUCUGGCCAG | 19 | 3316 |
| CCR5-3883 | − | UGCACAGCUCAUCUGGCCAG | 20 | 3317 |
| CCR5-3884 | − | UUGCACAGCUCAUCUGGCCAG | 21 | 3318 |
| CCR5-3885 | − | UUUGCACAGCUCAUCUGGCCAG | 22 | 3319 |
| CCR5-3886 | − | AUUUGCACAGCUCAUCUGGCCAG | 23 | 3320 |
| CCR5-3887 | − | GAUUUGCACAGCUCAUCUGGCCAG | 24 | 3321 |
| CCR5-3888 | − | CUCAUCUGGCCAGAAGAG | 18 | 3322 |
| CCR5-3889 | − | GCUCAUCUGGCCAGAAGAG | 19 | 3323 |
| CCR5-3890 | − | AGCUCAUCUGGCCAGAAGAG | 20 | 3324 |
| CCR5-3891 | − | CAGCUCAUCUGGCCAGAAGAG | 21 | 3325 |
| CCR5-3892 | − | ACAGCUCAUCUGGCCAGAAGAG | 22 | 3326 |
| CCR5-3893 | − | CACAGCUCAUCUGGCCAGAAGAG | 23 | 3327 |
| CCR5-3894 | − | GCACAGCUCAUCUGGCCAGAAGAG | 24 | 3328 |
| CCR5-3895 | − | UAGGGGAUACGGGGAGAG | 18 | 3329 |
| CCR5-3896 | − | AUAGGGGAUACGGGGAGAG | 19 | 3330 |
| CCR5-2819 | − | GAUAGGGGAUACGGGGAGAG | 20 | 3331 |
| CCR5-3897 | − | GGAUAGGGGAUACGGGGAGAG | 21 | 3332 |
| CCR5-3898 | − | GGGAUAGGGGAUACGGGGAGAG | 22 | 3333 |
| CCR5-3899 | − | UGGGAUAGGGGAUACGGGGAGAG | 23 | 3334 |
| CCR5-3900 | − | GUGGGAUAGGGGAUACGGGGAGAG | 24 | 3335 |
| CCR5-3901 | − | UCUGUGUAGUGGGAUGAG | 18 | 3336 |
| CCR5-3902 | − | UUCUGUGUAGUGGGAUGAG | 19 | 3337 |
| CCR5-3903 | − | AUUCUGUGUAGUGGGAUGAG | 20 | 3338 |
| CCR5-3904 | − | GAUUCUGUGUAGUGGGAUGAG | 21 | 3339 |
| CCR5-3905 | − | AGAUUCUGUGUAGUGGGAUGAG | 22 | 3340 |
| CCR5-3906 | − | CAGAUUCUGUGUAGUGGGAUGAG | 23 | 3341 |
| CCR5-3907 | − | ACAGAUUCUGUGUAGUGGGAUGAG | 24 | 3342 |
| CCR5-3908 | − | CAGAGAGAUGAGUCUUAG | 18 | 3343 |
| CCR5-3909 | − | GCAGAGAGAUGAGUCUUAG | 19 | 3344 |
| CCR5-3910 | − | UGCAGAGAGAUGAGUCUUAG | 20 | 3345 |
| CCR5-3911 | − | UUGCAGAGAGAUGAGUCUUAG | 21 | 3346 |
| CCR5-3912 | − | UUUGCAGAGAGAUGAGUCUUAG | 22 | 3347 |
| CCR5-3913 | − | AUUUGCAGAGAGAUGAGUCUUAG | 23 | 3348 |
| CCR5-3914 | − | GAUUUGCAGAGAGAUGAGUCUUAG | 24 | 3349 |
| CCR5-3915 | − | GGUGGGAUAGGGGAUACG | 18 | 3350 |
| CCR5-3916 | − | GGGUGGGAUAGGGGAUACG | 19 | 3351 |
| CCR5-2951 | − | GGGGUGGGAUAGGGGAUACG | 20 | 3352 |
| CCR5-3917 | − | UGGGGUGGGAUAGGGGAUACG | 21 | 3353 |
| CCR5-3918 | − | UUGGGGUGGGAUAGGGGAUACG | 22 | 3354 |
| CCR5-3919 | − | GUUGGGGUGGGAUAGGGGAUACG | 23 | 3355 |
| CCR5-3920 | − | GGUUGGGGUGGGAUAGGGGAUACG | 24 | 3356 |
| CCR5-3921 | − | UGAGCAUCUGUGUGGGGG | 18 | 3357 |
| CCR5-3922 | − | GUGAGCAUCUGUGUGGGGG | 19 | 3358 |
| CCR5-3923 | − | GGUGAGCAUCUGUGUGGGGG | 20 | 3359 |
| CCR5-3924 | − | UGGUGAGCAUCUGUGUGGGGG | 21 | 3360 |
| CCR5-3925 | − | GUGGUGAGCAUCUGUGUGGGGG | 22 | 3361 |
| CCR5-3926 | − | GGUGGUGAGCAUCUGUGUGGGGG | 23 | 3362 |
| CCR5-3927 | − | GGGUGGUGAGCAUCUGUGUGGGGG | 24 | 3363 |
| CCR5-3928 | − | CAGAUUCUGUGUAGUGGG | 18 | 3364 |
| CCR5-3929 | − | ACAGAUUCUGUGUAGUGGG | 19 | 3365 |
| CCR5-3930 | − | AACAGAUUCUGUGUAGUGGG | 20 | 3366 |
| CCR5-3931 | − | UAACAGAUUCUGUGUAGUGGG | 21 | 3367 |
| CCR5-3932 | − | CUAACAGAUUCUGUGUAGUGGG | 22 | 3368 |
| CCR5-3933 | − | UCUAACAGAUUCUGUGUAGUGGG | 23 | 3369 |
| CCR5-3934 | − | UUCUAACAGAUUCUGUGUAGUGGG | 24 | 3370 |
| CCR5-3935 | − | AUCUGUGUGGGGGUUGGG | 18 | 3371 |
| CCR5-3936 | − | CAUCUGUGUGGGGGUUGGG | 19 | 3372 |
| CCR5-3937 | − | GCAUCUGUGUGGGGGUUGGG | 20 | 3373 |
| CCR5-3938 | − | AGCAUCUGUGUGGGGGUUGGG | 21 | 3374 |
| CCR5-3939 | − | GAGCAUCUGUGUGGGGGUUGGG | 22 | 3375 |
| CCR5-3940 | − | UGAGCAUCUGUGUGGGGGUUGGG | 23 | 3376 |
| CCR5-3941 | − | GUGAGCAUCUGUGUGGGGGUUGGG | 24 | 3377 |
| CCR5-3942 | − | AACCUUUUAGCCUUACUG | 18 | 3378 |
| CCR5-3943 | − | UAACCUUUUAGCCUUACUG | 19 | 3379 |
| CCR5-3944 | − | UUAACCUUUUAGCCUUACUG | 20 | 3380 |
| CCR5-3945 | − | CUUAACCUUUUAGCCUUACUG | 21 | 3381 |
| CCR5-3946 | − | UCUUAACCUUUUAGCCUUACUG | 22 | 3382 |
| CCR5-3947 | − | UUCUUAACCUUUUAGCCUUACUG | 23 | 3383 |
| CCR5-3948 | − | UUUCUUAACCUUUUAGCCUUACUG | 24 | 3384 |
| CCR5-3949 | − | GGGGAUACGGGGAGAGUG | 18 | 3385 |
| CCR5-3950 | − | AGGGGAUACGGGGAGAGUG | 19 | 3386 |
| CCR5-3951 | − | UAGGGGAUACGGGGAGAGUG | 20 | 3387 |
| CCR5-3952 | − | AUAGGGGAUACGGGGAGAGUG | 21 | 3388 |
| CCR5-3953 | − | GAUAGGGGAUACGGGGAGAGUG | 22 | 3389 |
| CCR5-3954 | − | GGAUAGGGGAUACGGGGAGAGUG | 23 | 3390 |
| CCR5-3955 | − | GGGAUAGGGGAUACGGGGAGAGUG | 24 | 3391 |
| CCR5-3956 | − | GAACAAUAAUAUUGGGUG | 18 | 3392 |
| CCR5-3957 | − | AGAACAAUAAUAUUGGGUG | 19 | 3393 |
| CCR5-3958 | − | GAGAACAAUAAUAUUGGGUG | 20 | 3394 |
| CCR5-3959 | − | AGAGAACAAUAAUAUUGGGUG | 21 | 3395 |
| CCR5-3960 | − | CAGAGAACAAUAAUAUUGGGUG | 22 | 3396 |
| CCR5-3961 | − | ACAGAGAACAAUAAUAUUGGGUG | 23 | 3397 |
| CCR5-3962 | − | UACAGAGAACAAUAAUAUUGGGUG | 24 | 3398 |
| CCR5-3963 | − | GGGUGGUGAGCAUCUGUG | 18 | 3399 |
| CCR5-3964 | − | UGGGUGGUGAGCAUCUGUG | 19 | 3400 |
| CCR5-2959 | − | UUGGGUGGUGAGCAUCUGUG | 20 | 3401 |
| CCR5-3965 | − | AUUGGGUGGUGAGCAUCUGUG | 21 | 3402 |
| CCR5-3966 | − | UAUUGGGUGGUGAGCAUCUGUG | 22 | 3403 |
| CCR5-3967 | − | AUAUUGGGUGGUGAGCAUCUGUG | 23 | 3404 |
| CCR5-3968 | − | AAUAUUGGGUGGUGAGCAUCUGUG | 24 | 3405 |
| CCR5-3969 | − | UCUCAAAAGAAAGAUUUG | 18 | 3406 |
| CCR5-3970 | − | CUCUCAAAAGAAAGAUUUG | 19 | 3407 |
| CCR5-3971 | − | CCUCUCAAAAGAAAGAUUUG | 20 | 3408 |
| CCR5-3972 | − | ACCUCUCAAAAGAAAGAUUUG | 21 | 3409 |
| CCR5-3973 | − | UACCUCUCAAAAGAAAGAUUUG | 22 | 3410 |
| CCR5-3974 | − | UUACCUCUCAAAAGAAAGAUUUG | 23 | 3411 |
| CCR5-3975 | − | CUUACCUCUCAAAAGAAAGAUUUG | 24 | 3412 |
| CCR5-3976 | − | AAUUUCUUUUACUAAAAU | 18 | 3413 |
| CCR5-3977 | − | UAAUUUCUUUUACUAAAAU | 19 | 3414 |
| CCR5-3978 | − | GUAAUUUCUUUUACUAAAAU | 20 | 3415 |
| CCR5-3979 | − | AGUAAUUUCUUUUACUAAAAU | 21 | 3416 |
| CCR5-3980 | − | UAGUAAUUUCUUUUACUAAAAU | 22 | 3417 |
| CCR5-3981 | − | AUAGUAAUUUCUUUUACUAAAAU | 23 | 3418 |
| CCR5-3982 | − | GAUAGUAAUUUCUUUUACUAAAAU | 24 | 3419 |
| CCR5-3983 | − | AGGGGACACAGGGUUAAU | 18 | 3420 |
| CCR5-3984 | − | AAGGGGACACAGGGUUAAU | 19 | 3421 |
| CCR5-3985 | − | AAAGGGGACACAGGGUUAAU | 20 | 3422 |
| CCR5-3986 | − | AAAAGGGGACACAGGGUUAAU | 21 | 3423 |
| CCR5-3987 | − | AAAAAGGGGACACAGGGUUAAU | 22 | 3424 |
| CCR5-3988 | − | GAAAAAGGGGACACAGGGUUAAU | 23 | 3425 |
| CCR5-3989 | − | AGAAAAAGGGGACACAGGGUUAAU | 24 | 3426 |
| CCR5-3990 | − | AAAUAUAAUCUUUAAGAU | 18 | 3427 |
| CCR5-3991 | − | AAAAUAUAAUCUUUAAGAU | 19 | 3428 |
| CCR5-3992 | − | UAAAAUAUAAUCUUUAAGAU | 20 | 3429 |
| CCR5-3993 | − | UUAAAAUAUAAUCUUUAAGAU | 21 | 3430 |
| CCR5-3994 | − | CUUAAAAUAUAAUCUUUAAGAU | 22 | 3431 |
| CCR5-3995 | − | UCUUAAAAUAUAAUCUUUAAGAU | 23 | 3432 |
| CCR5-3996 | − | AUCUUAAAAUAUAAUCUUUAAGAU | 24 | 3433 |
| CCR5-3997 | − | UGGGGUGGGAUAGGGGAU | 18 | 3434 |
| CCR5-3998 | − | UUGGGGUGGGAUAGGGGAU | 19 | 3435 |
| CCR5-3999 | − | GUUGGGGUGGGAUAGGGGAU | 20 | 3436 |
| CCR5-4000 | − | GGUUGGGGUGGGAUAGGGGAU | 21 | 3437 |
| CCR5-4001 | − | GGGUUGGGGUGGGAUAGGGGAU | 22 | 3438 |
| CCR5-4002 | − | GGGGUUGGGGUGGGAUAGGGGAU | 23 | 3439 |
| CCR5-4003 | − | GGGGGUUGGGGUGGGAUAGGGGAU | 24 | 3440 |
| CCR5-4004 | − | UGGGGGUUGGGGUGGGAU | 18 | 3441 |
| CCR5-4005 | − | GUGGGGGUUGGGGUGGGAU | 19 | 3442 |
| CCR5-2962 | − | UGUGGGGGUUGGGGUGGGAU | 20 | 3443 |
| CCR5-4006 | − | GUGUGGGGGUUGGGGUGGGAU | 21 | 3444 |
| CCR5-4007 | − | UGUGUGGGGGUUGGGGUGGGAU | 22 | 3445 |
| CCR5-4008 | − | CUGUGUGGGGGUUGGGGUGGGAU | 23 | 3446 |
| CCR5-4009 | − | UCUGUGUGGGGGUUGGGGUGGGAU | 24 | 3447 |
| CCR5-4010 | − | GAAAUCUUAUCUUCUGCU | 18 | 3448 |
| CCR5-4011 | − | UGAAAUCUUAUCUUCUGCU | 19 | 3449 |
| CCR5-4012 | − | UUGAAAUCUUAUCUUCUGCU | 20 | 3450 |
| CCR5-4013 | − | CUUGAAAUCUUAUCUUCUGCU | 21 | 3451 |
| CCR5-4014 | − | UCUUGAAAUCUUAUCUUCUGCU | 22 | 3452 |
| CCR5-4015 | − | AUCUUGAAAUCUUAUCUUCUGCU | 23 | 3453 |
| CCR5-4016 | − | AAUCUUGAAAUCUUAUCUUCUGCU | 24 | 3454 |
| CCR5-4017 | − | UAAGGAAAGGGUCACAGU | 18 | 3455 |
| CCR5-4018 | − | AUAAGGAAAGGGUCACAGU | 19 | 3456 |
| CCR5-4019 | − | GAUAAGGAAAGGGUCACAGU | 20 | 3457 |
| CCR5-4020 | − | AGAUAAGGAAAGGGUCACAGU | 21 | 3458 |
| CCR5-4021 | − | AAGAUAAGGAAAGGGUCACAGU | 22 | 3459 |
| CCR5-4022 | − | UAAGAUAAGGAAAGGGUCACAGU | 23 | 3460 |
| CCR5-4023 | − | UUAAGAUAAGGAAAGGGUCACAGU | 24 | 3461 |
| CCR5-4024 | − | AAAACAAAAUAAUCCAGU | 18 | 3462 |
| CCR5-4025 | − | AAAAACAAAAUAAUCCAGU | 19 | 3463 |
| CCR5-4026 | − | CAAAAACAAAAUAAUCCAGU | 20 | 3464 |
| CCR5-4027 | − | ACAAAAACAAAAUAAUCCAGU | 21 | 3465 |
| CCR5-4028 | − | AACAAAAACAAAAUAAUCCAGU | 22 | 3466 |
| CCR5-4029 | − | GAACAAAAACAAAAUAAUCCAGU | 23 | 3467 |
| CCR5-4030 | − | AGAACAAAAACAAAAUAAUCCAGU | 24 | 3468 |
| CCR5-4031 | − | UGGGUGGUGAGCAUCUGU | 18 | 3469 |
| CCR5-4032 | − | UUGGGUGGUGAGCAUCUGU | 19 | 3470 |
| CCR5-4033 | − | AUUGGGUGGUGAGCAUCUGU | 20 | 3471 |
| CCR5-4034 | − | UAUUGGGUGGUGAGCAUCUGU | 21 | 3472 |
| CCR5-4035 | − | AUAUUGGGUGGUGAGCAUCUGU | 22 | 3473 |
| CCR5-4036 | − | AAUAUUGGGUGGUGAGCAUCUGU | 23 | 3474 |
| CCR5-4037 | − | UAAUAUUGGGUGGUGAGCAUCUGU | 24 | 3475 |
| CCR5-4038 | − | UGGCCUGUUAGUUAGCUU | 18 | 3476 |
| CCR5-4039 | − | UUGGCCUGUUAGUUAGCUU | 19 | 3477 |
| CCR5-4040 | − | CUUGGCCUGUUAGUUAGCUU | 20 | 3478 |
| CCR5-4041 | − | GCUUGGCCUGUUAGUUAGCUU | 21 | 3479 |
| CCR5-4042 | − | UGCUUGGCCUGUUAGUUAGCUU | 22 | 3480 |
| CCR5-4043 | − | CUGCUUGGCCUGUUAGUUAGCUU | 23 | 3481 |
| CCR5-4044 | − | GCUGCUUGGCCUGUUAGUUAGCUU | 24 | 3482 |

Table 6E provides exemplary targeting domains for knocking down the CCR5 gene selected according to the fifth tier parameters. Within the additional 500 bp (e.g., upstream or downstream) of a transcription start site (TSS), e.g., extending to 1 kb upstream and downstream of a TSS and PAM is NNGRRV. It is contemplated herein that in an embodiment the targeting domain hybridizes to the target domain through complementary base pairing. Any of the targeting domains in the table can be used with a S. aureus eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 6E
5th Tier

| gRNA | DNA | | Target Site | |
|---|---|---|---|---|
| Name | Strand | Targeting Domain | Length | SEQ ID NO |
| CCR5-4208 | + | UGGAGGAAAAAGAAAAAA | 18 | 3651 |
| CCR5-4209 | + | CUGGAGGAAAAAGAAAAAA | 19 | 3652 |
| CCR5-4210 | + | UCUGGAGGAAAAAGAAAAAA | 20 | 3653 |
| CCR5-4211 | + | GUCUGGAGGAAAAAGAAAAAA | 21 | 3654 |
| CCR5-4212 | + | UGUCUGGAGGAAAAAGAAAAAA | 22 | 3655 |
| CCR5-4213 | + | UUGUCUGGAGGAAAAAGAAAAAA | 23 | 3656 |
| CCR5-4214 | + | CUUGUCUGGAGGAAAAAGAAAAAA | 24 | 3657 |
| CCR5-4215 | + | UCUGGAGGAAAAAGAAAA | 18 | 3658 |
| CCR5-4216 | + | GUCUGGAGGAAAAAGAAAA | 19 | 3659 |
| CCR5-4217 | + | UGUCUGGAGGAAAAAGAAAA | 20 | 3660 |
| CCR5-4218 | + | UUGUCUGGAGGAAAAAGAAAA | 21 | 3661 |
| CCR5-4219 | + | CUUGUCUGGAGGAAAAAGAAAA | 22 | 3662 |
| CCR5-4220 | + | UCUUGUCUGGAGGAAAAAGAAAA | 23 | 3663 |
| CCR5-4221 | + | CUCUUGUCUGGAGGAAAAAGAAAA | 24 | 3664 |
| CCR5-4222 | + | CCUCUUGUCUGGAGGAAA | 18 | 3665 |
| CCR5-4223 | + | CCCUCUUGUCUGGAGGAAA | 19 | 3666 |
| CCR5-4224 | + | UCCCUCUUGUCUGGAGGAAA | 20 | 3667 |
| CCR5-4225 | + | UUCCCUCUUGUCUGGAGGAAA | 21 | 3668 |
| CCR5-4226 | + | CUUCCCUCUUGUCUGGAGGAAA | 22 | 3669 |
| CCR5-4227 | + | GCUUCCCUCUUGUCUGGAGGAAA | 23 | 3670 |
| CCR5-4228 | + | GGCUUCCCUCUUGUCUGGAGGAAA | 24 | 3671 |
| CCR5-4229 | + | GAUGUCACCAACCGCCAA | 18 | 3672 |
| CCR5-4230 | + | AGAUGUCACCAACCGCCAA | 19 | 3673 |
| CCR5-4231 | + | CAGAUGUCACCAACCGCCAA | 20 | 3674 |
| CCR5-4232 | + | UCAGAUGUCACCAACCGCCAA | 21 | 3675 |
| CCR5-4233 | + | UUCAGAUGUCACCAACCGCCAA | 22 | 3676 |
| CCR5-4234 | + | UUUCAGAUGUCACCAACCGCCAA | 23 | 3677 |
| CCR5-4235 | + | UUUUCAGAUGUCACCAACCGCCAA | 24 | 3678 |
| CCR5-4236 | + | CAAGGUCACGGAAGCCCA | 18 | 3679 |
| CCR5-4237 | + | CCAAGGUCACGGAAGCCCA | 19 | 3680 |
| CCR5-4238 | + | GCCAAGGUCACGGAAGCCCA | 20 | 3681 |
| CCR5-4239 | + | AGCCAAGGUCACGGAAGCCCA | 21 | 3682 |
| CCR5-4240 | + | GAGCCAAGGUCACGGAAGCCCA | 22 | 3683 |
| CCR5-4241 | + | AGAGCCAAGGUCACGGAAGCCCA | 23 | 3684 |
| CCR5-4242 | + | UAGAGCCAAGGUCACGGAAGCCCA | 24 | 3685 |
| CCR5-4243 | + | AUUCUAGAGCCAAGGUCA | 18 | 3686 |
| CCR5-4244 | + | UAUUCUAGAGCCAAGGUCA | 19 | 3687 |
| CCR5-3069 | + | UUAUUCUAGAGCCAAGGUCA | 20 | 3688 |
| CCR5-4245 | + | UUUAUUCUAGAGCCAAGGUCA | 21 | 3689 |
| CCR5-4246 | + | UUUUAUUCUAGAGCCAAGGUCA | 22 | 3690 |
| CCR5-4247 | + | UUUUUAUUCUAGAGCCAAGGUCA | 23 | 3691 |
| CCR5-4248 | + | CUUUUUAUUCUAGAGCCAAGGUCA | 24 | 3692 |
| CCR5-4249 | + | CCUGGGUCCAGAAAAAGA | 18 | 3693 |
| CCR5-4250 | + | UCCUGGGUCCAGAAAAAGA | 19 | 3694 |
| CCR5-3071 | + | AUCCUGGGUCCAGAAAAAGA | 20 | 3695 |
| CCR5-4251 | + | GAUCCUGGGUCCAGAAAAAGA | 21 | 3696 |
| CCR5-4252 | + | AGAUCCUGGGUCCAGAAAAAGA | 22 | 3697 |
| CCR5-4253 | + | AAGAUCCUGGGUCCAGAAAAAGA | 23 | 3698 |
| CCR5-4254 | + | UAAGAUCCUGGGUCCAGAAAAAGA | 24 | 3699 |
| CCR5-4255 | + | AACAAAAUAGUGAACAGA | 18 | 3700 |
| CCR5-4256 | + | CAACAAAAUAGUGAACAGA | 19 | 3701 |
| CCR5-4257 | + | GCAACAAAAUAGUGAACAGA | 20 | 3702 |
| CCR5-4258 | + | GGCAACAAAAUAGUGAACAGA | 21 | 3703 |
| CCR5-4259 | + | GGGCAACAAAAUAGUGAACAGA | 22 | 3704 |
| CCR5-4260 | + | AGGGCAACAAAAUAGUGAACAGA | 23 | 3705 |
| CCR5-4261 | + | AAGGGCAACAAAAUAGUGAACAGA | 24 | 3706 |
| CCR5-4262 | + | AGAUAGAUUAUAUCUGGA | 18 | 3707 |
| CCR5-4263 | + | CAGAUAGAUUAUAUCUGGA | 19 | 3708 |
| CCR5-4264 | + | UCAGAUAGAUUAUAUCUGGA | 20 | 3709 |
| CCR5-4265 | + | UUCAGAUAGAUUAUAUCUGGA | 21 | 3710 |
| CCR5-4266 | + | CUUCAGAUAGAUUAUAUCUGGA | 22 | 3711 |
| CCR5-4267 | + | GCUUCAGAUAGAUUAUAUCUGGA | 23 | 3712 |
| CCR5-4268 | + | AGCUUCAGAUAGAUUAUAUCUGGA | 24 | 3713 |
| CCR5-4269 | + | CUUAGACUAGGCAGCUGA | 18 | 3714 |
| CCR5-4270 | + | CCUUAGACUAGGCAGCUGA | 19 | 3715 |
| CCR5-4271 | + | ACCUUAGACUAGGCAGCUGA | 20 | 3716 |
| CCR5-4272 | + | CACCUUAGACUAGGCAGCUGA | 21 | 3717 |
| CCR5-4273 | + | GCACCUUAGACUAGGCAGCUGA | 22 | 3718 |
| CCR5-4274 | + | UGCACCUUAGACUAGGCAGCUGA | 23 | 3719 |
| CCR5-4275 | + | CUGCACCUUAGACUAGGCAGCUGA | 24 | 3720 |
| CCR5-4276 | + | UUGAAGGGCAACAAAAUA | 18 | 3721 |
| CCR5-4277 | + | UUUGAAGGGCAACAAAAUA | 19 | 3722 |
| CCR5-4278 | + | GUUUGAAGGGCAACAAAAUA | 20 | 3723 |
| CCR5-4279 | + | GGUUUGAAGGGCAACAAAAUA | 21 | 3724 |
| CCR5-4280 | + | UGGUUUGAAGGGCAACAAAAUA | 22 | 3725 |
| CCR5-4281 | + | CUGGUUUGAAGGGCAACAAAAUA | 23 | 3726 |
| CCR5-4282 | + | ACUGGUUUGAAGGGCAACAAAAUA | 24 | 3727 |
| CCR5-4283 | + | GUAUAUAGUAUAGUCAUA | 18 | 3728 |
| CCR5-4284 | + | UGUAUAUAGUAUAGUCAUA | 19 | 3729 |
| CCR5-4285 | + | CUGUAUAUAGUAUAGUCAUA | 20 | 3730 |
| CCR5-4286 | + | ACUGUAUAUAGUAUAGUCAUA | 21 | 3731 |
| CCR5-4287 | + | GACUGUAUAUAGUAUAGUCAUA | 22 | 3732 |
| CCR5-4288 | + | UGACUGUAUAUAGUAUAGUCAUA | 23 | 3733 |
| CCR5-4289 | + | AUGACUGUAUAUAGUAUAGUCAUA | 24 | 3734 |
| CCR5-4290 | + | CAUGAAACUGAUAUAUUA | 18 | 3735 |
| CCR5-4291 | + | CCAUGAAACUGAUAUAUUA | 19 | 3736 |
| CCR5-4292 | + | GCCAUGAAACUGAUAUAUUA | 20 | 3737 |
| CCR5-4293 | + | UGCCAUGAAACUGAUAUAUUA | 21 | 3738 |
| CCR5-4294 | + | GUGCCAUGAAACUGAUAUAUUA | 22 | 3739 |
| CCR5-4295 | + | UGUGCCAUGAAACUGAUAUAUUA | 23 | 3740 |
| CCR5-4296 | + | CUGUGCCAUGAAACUGAUAUAUUA | 24 | 3741 |
| CCR5-4297 | + | AGUAUAGUCAUAAAGAAC | 18 | 3742 |
| CCR5-4298 | + | UAGUAUAGUCAUAAAGAAC | 19 | 3743 |
| CCR5-4299 | + | AUAGUAUAGUCAUAAAGAAC | 20 | 3744 |
| CCR5-4300 | + | UAUAGUAUAGUCAUAAAGAAC | 21 | 3745 |
| CCR5-4301 | + | AUAUAGUAUAGUCAUAAAGAAC | 22 | 3746 |
| CCR5-4302 | + | UAUAUAGUAUAGUCAUAAAGAAC | 23 | 3747 |
| CCR5-4303 | + | GUAUAUAGUAUAGUCAUAAAGAAC | 24 | 3748 |
| CCR5-4304 | + | CAGCUCUGCUGACAAUAC | 18 | 3749 |
| CCR5-4305 | + | UCAGCUCUGCUGACAAUAC | 19 | 3750 |
| CCR5-4306 | + | CUCAGCUCUGCUGACAAUAC | 20 | 3751 |
| CCR5-4307 | + | UCUCAGCUCUGCUGACAAUAC | 21 | 3752 |
| CCR5-4308 | + | UUCUCAGCUCUGCUGACAAUAC | 22 | 3753 |
| CCR5-4309 | + | CUUCUCAGCUCUGCUGACAAUAC | 23 | 3754 |
| CCR5-4310 | + | UCUUCUCAGCUCUGCUGACAAUAC | 24 | 3755 |
| CCR5-4311 | + | AACCUGUUUAGCUCACCC | 18 | 3756 |
| CCR5-4312 | + | AAACCUGUUUAGCUCACCC | 19 | 3757 |
| CCR5-4313 | + | GAAACCUGUUUAGCUCACCC | 20 | 3758 |
| CCR5-4314 | + | GGAAACCUGUUUAGCUCACCC | 21 | 3759 |
| CCR5-4315 | + | GGGAAACCUGUUUAGCUCACCC | 22 | 3760 |
| CCR5-4316 | + | UGGGAAACCUGUUUAGCUCACCC | 23 | 3761 |
| CCR5-4317 | + | AUGGGAAACCUGUUUAGCUCACCC | 24 | 3762 |
| CCR5-4318 | + | GAGUUGUCAUACAUACCC | 18 | 3763 |
| CCR5-4319 | + | AGAGUUGUCAUACAUACCC | 19 | 3764 |
| CCR5-4320 | + | AAGAGUUGUCAUACAUACCC | 20 | 3765 |
| CCR5-4321 | + | UAAGAGUUGUCAUACAUACCC | 21 | 3766 |
| CCR5-4322 | + | UUAAGAGUUGUCAUACAUACCC | 22 | 3767 |
| CCR5-4323 | + | AUUAAGAGUUGUCAUACAUACCC | 23 | 3768 |
| CCR5-4324 | + | AAUUAAGAGUUGUCAUACAUACCC | 24 | 3769 |
| CCR5-4325 | + | GCAGCUGAGAGAAGCCCC | 18 | 3770 |
| CCR5-4326 | + | GGCAGCUGAGAGAAGCCCC | 19 | 3771 |
| CCR5-4327 | + | AGGCAGCUGAGAGAAGCCCC | 20 | 3772 |
| CCR5-4328 | + | UAGGCAGCUGAGAGAAGCCCC | 21 | 3773 |
| CCR5-4329 | + | CUAGGCAGCUGAGAGAAGCCCC | 22 | 3774 |
| CCR5-4330 | + | ACUAGGCAGCUGAGAGAAGCCCC | 23 | 3775 |
| CCR5-4331 | + | GACUAGGCAGCUGAGAGAAGCCCC | 24 | 3776 |
| CCR5-4332 | + | GCCAAGGUCACGGAAGCC | 18 | 3777 |
| CCR5-4333 | + | AGCCAAGGUCACGGAAGCC | 19 | 3778 |
| CCR5-4334 | + | GAGCCAAGGUCACGGAAGCC | 20 | 3779 |
| CCR5-4335 | + | AGAGCCAAGGUCACGGAAGCC | 21 | 3780 |
| CCR5-4336 | + | UAGAGCCAAGGUCACGGAAGCC | 22 | 3781 |
| CCR5-4337 | + | CUAGAGCCAAGGUCACGGAAGCC | 23 | 3782 |
| CCR5-4338 | + | UCUAGAGCCAAGGUCACGGAAGCC | 24 | 3783 |
| CCR5-4339 | + | CAGAUGUCACCAACCGCC | 18 | 3784 |
| CCR5-4340 | + | UCAGAUGUCACCAACCGCC | 19 | 3785 |
| CCR5-4341 | + | UUCAGAUGUCACCAACCGCC | 20 | 3786 |
| CCR5-4342 | + | UUUCAGAUGUCACCAACCGCC | 21 | 3787 |
| CCR5-4343 | + | UUUUCAGAUGUCACCAACCGCC | 22 | 3788 |
| CCR5-4344 | + | AUUUUCAGAUGUCACCAACCGCC | 23 | 3789 |
| CCR5-4345 | + | GAUUUUCAGAUGUCACCAACCGCC | 24 | 3790 |
| CCR5-4346 | + | UUAUAUACUAACUGUGCC | 18 | 3791 |
| CCR5-4347 | + | AUUAUAUACUAACUGUGCC | 19 | 3792 |
| CCR5-4348 | + | AAUUAUAUACUAACUGUGCC | 20 | 3793 |
| CCR5-4349 | + | GAAUUAUAUACUAACUGUGCC | 21 | 3794 |
| CCR5-4350 | + | AGAAUUAUAUACUAACUGUGCC | 22 | 3795 |
| CCR5-4351 | + | AAGAAUUAUAUACUAACUGUGCC | 23 | 3796 |
| CCR5-4352 | + | AAAGAAUUAUAUACUAACUGUGCC | 24 | 3797 |
| CCR5-4353 | + | CAGAGGGCAUCUUGUGGC | 18 | 3798 |
| CCR5-4354 | + | CCAGAGGGCAUCUUGUGGC | 19 | 3799 |
| CCR5-4355 | + | CCCAGAGGGCAUCUUGUGGC | 20 | 3800 |
| CCR5-4356 | + | GCCCAGAGGGCAUCUUGUGGC | 21 | 3801 |
| CCR5-4357 | + | AGCCCAGAGGGCAUCUUGUGGC | 22 | 3802 |
| CCR5-4358 | + | AAGCCCAGAGGGCAUCUUGUGGC | 23 | 3803 |
| CCR5-4359 | + | GAAGCCCAGAGGGCAUCUUGUGGC | 24 | 3804 |
| CCR5-4360 | + | UAUUCUAGAGCCAAGGUC | 18 | 3805 |
| CCR5-4361 | + | UUAUUCUAGAGCCAAGGUC | 19 | 3806 |
| CCR5-4362 | + | UUUAUUCUAGAGCCAAGGUC | 20 | 3807 |
| CCR5-4363 | + | UUUUAUUCUAGAGCCAAGGUC | 21 | 3808 |
| CCR5-4364 | + | UUUUUAUUCUAGAGCCAAGGUC | 22 | 3809 |
| CCR5-4365 | + | CUUUUUAUUCUAGAGCCAAGGUC | 23 | 3810 |
| CCR5-4366 | + | GCUUUUUAUUCUAGAGCCAAGGUC | 24 | 3811 |
| CCR5-4367 | + | CCACUAAGAUCCUGGGUC | 18 | 3812 |
| CCR5-4368 | + | CCCACUAAGAUCCUGGGUC | 19 | 3813 |
| CCR5-4369 | + | CCCCACUAAGAUCCUGGGUC | 20 | 3814 |
| CCR5-4370 | + | UCCCCACUAAGAUCCUGGGUC | 21 | 3815 |
| CCR5-4371 | + | AUCCCCACUAAGAUCCUGGGUC | 22 | 3816 |
| CCR5-4372 | + | AAUCCCCACUAAGAUCCUGGGUC | 23 | 3817 |
| CCR5-4373 | + | AAAUCCCCACUAAGAUCCUGGGUC | 24 | 3818 |
| CCR5-4374 | + | UUAGGCUUCCCUCUUGUC | 18 | 3819 |
| CCR5-4375 | + | UUUAGGCUUCCCUCUUGUC | 19 | 3820 |
| CCR5-3097 | + | UUUUAGGCUUCCCUCUUGUC | 20 | 3821 |
| CCR5-4376 | + | UUUUUAGGCUUCCCUCUUGUC | 21 | 3822 |
| CCR5-4377 | + | AUUUUUAGGCUUCCCUCUUGUC | 22 | 3823 |
| CCR5-4378 | + | CAUUUUUAGGCUUCCCUCUUGUC | 23 | 3824 |
| CCR5-4379 | + | CCAUUUUUAGGCUUCCCUCUUGUC | 24 | 3825 |
| CCR5-4380 | + | AGCCAAAGCUUUUUAUUC | 18 | 3826 |
| CCR5-4381 | + | AAGCCAAAGCUUUUUAUUC | 19 | 3827 |
| CCR5-4382 | + | CAAGCCAAAGCUUUUUAUUC | 20 | 3828 |
| CCR5-4383 | + | ACAAGCCAAAGCUUUUUAUUC | 21 | 3829 |
| CCR5-4384 | + | CACAAGCCAAAGCUUUUUAUUC | 22 | 3830 |
| CCR5-4385 | + | UCACAAGCCAAAGCUUUUUAUUC | 23 | 3831 |
| CCR5-4386 | + | AUCACAAGCCAAAGCUUUUUAUUC | 24 | 3832 |
| CCR5-4387 | + | UCCUGGGUCCAGAAAAAG | 18 | 3833 |
| CCR5-4388 | + | AUCCUGGGUCCAGAAAAAG | 19 | 3834 |
| CCR5-4389 | + | GAUCCUGGGUCCAGAAAAAG | 20 | 3835 |
| CCR5-4390 | + | AGAUCCUGGGUCCAGAAAAAG | 21 | 3836 |
| CCR5-4391 | + | AAGAUCCUGGGUCCAGAAAAAG | 22 | 3837 |
| CCR5-4392 | + | UAAGAUCCUGGGUCCAGAAAAAG | 23 | 3838 |
| CCR5-4393 | + | CUAAGAUCCUGGGUCCAGAAAAAG | 24 | 3839 |
| CCR5-4394 | + | GCACCUUAGACUAGGCAG | 18 | 3840 |
| CCR5-4395 | + | UGCACCUUAGACUAGGCAG | 19 | 3841 |
| CCR5-4396 | + | CUGCACCUUAGACUAGGCAG | 20 | 3842 |
| CCR5-4397 | + | CCUGCACCUUAGACUAGGCAG | 21 | 3843 |
| CCR5-4398 | + | CCCUGCACCUUAGACUAGGCAG | 22 | 3844 |
| CCR5-4399 | + | UCCCUGCACCUUAGACUAGGCAG | 23 | 3845 |
| CCR5-4400 | + | CUCCCUGCACCUUAGACUAGGCAG | 24 | 3846 |
| CCR5-4401 | + | UAAGUUCAGCUGCUCUAG | 18 | 3847 |
| CCR5-4402 | + | UUAAGUUCAGCUGCUCUAG | 19 | 3848 |
| CCR5-4403 | + | UUUAAGUUCAGCUGCUCUAG | 20 | 3849 |
| CCR5-4404 | + | AUUUAAGUUCAGCUGCUCUAG | 21 | 3850 |
| CCR5-4405 | + | UAUUUAAGUUCAGCUGCUCUAG | 22 | 3851 |
| CCR5-4406 | + | CUAUUUAAGUUCAGCUGCUCUAG | 23 | 3852 |
| CCR5-4407 | + | UCUAUUUAAGUUCAGCUGCUCUAG | 24 | 3853 |
| CCR5-4408 | + | AUGAAACUGAUAUAUUAG | 18 | 3854 |
| CCR5-4409 | + | CAUGAAACUGAUAUAUUAG | 19 | 3855 |
| CCR5-3105 | + | CCAUGAAACUGAUAUAUUAG | 20 | 3856 |
| CCR5-4410 | + | GCCAUGAAACUGAUAUAUUAG | 21 | 3857 |
| CCR5-4411 | + | UGCCAUGAAACUGAUAUAUUAG | 22 | 3858 |
| CCR5-4412 | + | GUGCCAUGAAACUGAUAUAUUAG | 23 | 3859 |
| CCR5-4413 | + | UGUGCCAUGAAACUGAUAUAUUAG | 24 | 3860 |
| CCR5-4414 | + | GGCUUCCCUCUUGUCUGG | 18 | 3861 |
| CCR5-4415 | + | AGGCUUCCCUCUUGUCUGG | 19 | 3862 |
| CCR5-3108 | + | UAGGCUUCCCUCUUGUCUGG | 20 | 3863 |
| CCR5-4416 | + | UUAGGCUUCCCUCUUGUCUGG | 21 | 3864 |
| CCR5-4417 | + | UUUAGGCUUCCCUCUUGUCUGG | 22 | 3865 |
| CCR5-4418 | + | UUUUAGGCUUCCCUCUUGUCUGG | 23 | 3866 |
| CCR5-4419 | + | UUUUUAGGCUUCCCUCUUGUCUGG | 24 | 3867 |
| CCR5-4420 | + | CCAUAUACUUAUGUCAUG | 18 | 3868 |
| CCR5-4421 | + | ACCAUAUACUUAUGUCAUG | 19 | 3869 |
| CCR5-3111 | + | GACCAUAUACUUAUGUCAUG | 20 | 3870 |
| CCR5-4422 | + | UGACCAUAUACUUAUGUCAUG | 21 | 3871 |
| CCR5-4423 | + | UUGACCAUAUACUUAUGUCAUG | 22 | 3872 |
| CCR5-4424 | + | CUUGACCAUAUACUUAUGUCAUG | 23 | 3873 |
| CCR5-4425 | + | ACUUGACCAUAUACUUAUGUCAUG | 24 | 3874 |
| CCR5-4426 | + | AGGCUUCCCUCUUGUCUG | 18 | 3875 |
| CCR5-4427 | + | UAGGCUUCCCUCUUGUCUG | 19 | 3876 |
| CCR5-4428 | + | UUAGGCUUCCCUCUUGUCUG | 20 | 3877 |
| CCR5-4429 | + | UUUAGGCUUCCCUCUUGUCUG | 21 | 3878 |
| CCR5-4430 | + | UUUUAGGCUUCCCUCUUGUCUG | 22 | 3879 |
| CCR5-4431 | + | UUUUUAGGCUUCCCUCUUGUCUG | 23 | 3880 |
| CCR5-4432 | + | AUUUUUAGGCUUCCCUCUUGUCUG | 24 | 3881 |
| CCR5-4433 | + | UAAAUGCUUACUGGUUUG | 18 | 3882 |
| CCR5-4434 | + | AUAAAUGCUUACUGGUUUG | 19 | 3883 |
| CCR5-4435 | + | CAUAAAUGCUUACUGGUUUG | 20 | 3884 |
| CCR5-4436 | + | UCAUAAAUGCUUACUGGUUUG | 21 | 3885 |
| CCR5-4437 | + | CUCAUAAAUGCUUACUGGUUUG | 22 | 3886 |
| CCR5-4438 | + | CCUCAUAAAUGCUUACUGGUUUG | 23 | 3887 |
| CCR5-4439 | + | UCCUCAUAAAUGCUUACUGGUUUG | 24 | 3888 |
| CCR5-4440 | + | ACCAUAUACUUAUGUCAU | 18 | 3889 |
| CCR5-4441 | + | GACCAUAUACUUAUGUCAU | 19 | 3890 |
| CCR5-4442 | + | UGACCAUAUACUUAUGUCAU | 20 | 3891 |
| CCR5-4443 | + | UUGACCAUAUACUUAUGUCAU | 21 | 3892 |
| CCR5-4444 | + | CUUGACCAUAUACUUAUGUCAU | 22 | 3893 |
| CCR5-4445 | + | ACUUGACCAUAUACUUAUGUCAU | 23 | 3894 |
| CCR5-4446 | + | AACUUGACCAUAUACUUAUGUCAU | 24 | 3895 |
| CCR5-4447 | + | CUGGGUCCAGAAAAAGAU | 18 | 3896 |
| CCR5-4448 | + | CCUGGGUCCAGAAAAAGAU | 19 | 3897 |
| CCR5-3122 | + | UCCUGGGUCCAGAAAAAGAU | 20 | 3898 |
| CCR5-4449 | + | AUCCUGGGUCCAGAAAAAGAU | 21 | 3899 |
| CCR5-4450 | + | GAUCCUGGGUCCAGAAAAAGAU | 22 | 3900 |
| CCR5-4451 | + | AGAUCCUGGGUCCAGAAAAAGAU | 23 | 3901 |
| CCR5-4452 | + | AAGAUCCUGGGUCCAGAAAAAGAU | 24 | 3902 |
| CCR5-4453 | + | GCCAUGAAACUGAUAUAU | 18 | 3903 |
| CCR5-4454 | + | UGCCAUGAAACUGAUAUAU | 19 | 3904 |
| CCR5-4455 | + | GUGCCAUGAAACUGAUAUAU | 20 | 3905 |
| CCR5-4456 | + | UGUGCCAUGAAACUGAUAUAU | 21 | 3906 |
| CCR5-4457 | + | CUGUGCCAUGAAACUGAUAUAU | 22 | 3907 |
| CCR5-4458 | + | ACUGUGCCAUGAAACUGAUAUAU | 23 | 3908 |
| CCR5-4459 | + | AACUGUGCCAUGAAACUGAUAUAU | 24 | 3909 |
| CCR5-4460 | + | GCUUCAGAUAGAUUAUAU | 18 | 3910 |
| CCR5-4461 | + | AGCUUCAGAUAGAUUAUAU | 19 | 3911 |
| CCR5-4462 | + | UAGCUUCAGAUAGAUUAUAU | 20 | 3912 |
| CCR5-4463 | + | AUAGCUUCAGAUAGAUUAUAU | 21 | 3913 |
| CCR5-4464 | + | CAUAGCUUCAGAUAGAUUAUAU | 22 | 3914 |
| CCR5-4465 | + | UCAUAGCUUCAGAUAGAUUAUAU | 23 | 3915 |
| CCR5-4466 | + | CUCAUAGCUUCAGAUAGAUUAUAU | 24 | 3916 |
| CCR5-4467 | + | ACCUUAGACUAGGCAGCU | 18 | 3917 |
| CCR5-4468 | + | CACCUUAGACUAGGCAGCU | 19 | 3918 |
| CCR5-4469 | + | GCACCUUAGACUAGGCAGCU | 20 | 3919 |
| CCR5-4470 | + | UGCACCUUAGACUAGGCAGCU | 21 | 3920 |
| CCR5-4471 | + | CUGCACCUUAGACUAGGCAGCU | 22 | 3921 |
| CCR5-4472 | + | CCUGCACCUUAGACUAGGCAGCU | 23 | 3922 |
| CCR5-4473 | + | CCCUGCACCUUAGACUAGGCAGCU | 24 | 3923 |
| CCR5-4474 | + | AGAGGGCAUCUUGUGGCU | 18 | 3924 |
| CCR5-4475 | + | CAGAGGGCAUCUUGUGGCU | 19 | 3925 |
| CCR5-3129 | + | CCAGAGGGCAUCUUGUGGCU | 20 | 3926 |
| CCR5-4476 | + | CCCAGAGGGCAUCUUGUGGCU | 21 | 3927 |
| CCR5-4477 | + | GCCCAGAGGGCAUCUUGUGGCU | 22 | 3928 |
| CCR5-4478 | + | AGCCCAGAGGGCAUCUUGUGGCU | 23 | 3929 |
| CCR5-4479 | + | AAGCCCAGAGGGCAUCUUGUGGCU | 24 | 3930 |
| CCR5-4480 | + | GGGUCUCAUUUGCCUUCU | 18 | 3931 |
| CCR5-4481 | + | GGGGUCUCAUUUGCCUUCU | 19 | 3932 |
| CCR5-4482 | + | UGGGGUCUCAUUUGCCUUCU | 20 | 3933 |
| CCR5-4483 | + | UUGGGGUCUCAUUUGCCUUCU | 21 | 3934 |
| CCR5-4484 | + | UUUGGGGUCUCAUUUGCCUUCU | 22 | 3935 |
| CCR5-4485 | + | GUUUGGGGUCUCAUUUGCCUUCU | 23 | 3936 |
| CCR5-4486 | + | UGUUUGGGGUCUCAUUUGCCUUCU | 24 | 3937 |
| CCR5-4487 | + | AAAAUCCUCACAUUUUCU | 18 | 3938 |
| CCR5-4488 | + | UAAAAUCCUCACAUUUUCU | 19 | 3939 |
| CCR5-4489 | + | GUAAAAUCCUCACAUUUUCU | 20 | 3940 |
| CCR5-4490 | + | UGUAAAAUCCUCACAUUUUCU | 21 | 3941 |
| CCR5-4491 | + | UUGUAAAAUCCUCACAUUUUCU | 22 | 3942 |
| CCR5-4492 | + | AUUGUAAAAUCCUCACAUUUUCU | 23 | 3943 |
| CCR5-4493 | + | AAUUGUAAAAUCCUCACAUUUUCU | 24 | 3944 |
| CCR5-4494 | + | UCAUAAAUGCUUACUGGU | 18 | 3945 |
| CCR5-4495 | + | CUCAUAAAUGCUUACUGGU | 19 | 3946 |
| CCR5-4496 | + | CCUCAUAAAUGCUUACUGGU | 20 | 3947 |
| CCR5-4497 | + | UCCUCAUAAAUGCUUACUGGU | 21 | 3948 |
| CCR5-4498 | + | GUCCUCAUAAAUGCUUACUGGU | 22 | 3949 |
| CCR5-4499 | + | AGUCCUCAUAAAUGCUUACUGGU | 23 | 3950 |
| CCR5-4500 | + | GAGUCCUCAUAAAUGCUUACUGGU | 24 | 3951 |
| CCR5-4501 | + | GGCACGUAAUUUUGCUGU | 18 | 3952 |
| CCR5-4502 | + | GGGCACGUAAUUUUGCUGU | 19 | 3953 |
| CCR5-4503 | + | GGGGCACGUAAUUUUGCUGU | 20 | 3954 |
| CCR5-4504 | + | GGGGGCACGUAAUUUUGCUGU | 21 | 3955 |
| CCR5-4505 | + | UGGGGGCACGUAAUUUUGCUGU | 22 | 3956 |
| CCR5-4506 | + | UUGGGGGCACGUAAUUUUGCUGU | 23 | 3957 |
| CCR5-4507 | + | AUUGGGGGCACGUAAUUUUGCUGU | 24 | 3958 |
| CCR5-4508 | + | UUUAGGCUUCCCUCUUGU | 18 | 3959 |
| CCR5-4509 | + | UUUUAGGCUUCCCUCUUGU | 19 | 3960 |
| CCR5-4510 | + | UUUUUAGGCUUCCCUCUUGU | 20 | 3961 |
| CCR5-4511 | + | AUUUUUAGGCUUCCCUCUUGU | 21 | 3962 |
| CCR5-4512 | + | CAUUUUUAGGCUUCCCUCUUGU | 22 | 3963 |
| CCR5-4513 | + | CCAUUUUUAGGCUUCCCUCUUGU | 23 | 3964 |
| CCR5-4514 | + | ACCAUUUUUAGGCUUCCCUCUUGU | 24 | 3965 |
| CCR5-4515 | + | AAAAGCUCAUUUUUAAUU | 18 | 3966 |
| CCR5-4516 | + | GAAAAGCUCAUUUUUAAUU | 19 | 3967 |
| CCR5-4517 | + | AGAAAAGCUCAUUUUUAAUU | 20 | 3968 |
| CCR5-4518 | + | UAGAAAAGCUCAUUUUUAAUU | 21 | 3969 |
| CCR5-4519 | + | CUAGAAAAGCUCAUUUUUAAUU | 22 | 3970 |
| CCR5-4520 | + | CCUAGAAAAGCUCAUUUUUAAUU | 23 | 3971 |
| CCR5-4521 | + | CCCUAGAAAAGCUCAUUUUUAAUU | 24 | 3972 |
| CCR5-4522 | + | ACUUAGACACAACUUCUU | 18 | 3973 |
| CCR5-4523 | + | GACUUAGACACAACUUCUU | 19 | 3974 |
| CCR5-4524 | + | AGACUUAGACACAACUUCUU | 20 | 3975 |
| CCR5-4525 | + | CAGACUUAGACACAACUUCUU | 21 | 3976 |
| CCR5-4526 | + | CCAGACUUAGACACAACUUCUU | 22 | 3977 |
| CCR5-4527 | + | ACCAGACUUAGACACAACUUCUU | 23 | 3978 |
| CCR5-4528 | + | AACCAGACUUAGACACAACUUCUU | 24 | 3979 |
| CCR5-4529 | − | UAUGGUUCAAAAUUAAAA | 18 | 3980 |
| CCR5-4530 | − | UUAUGGUUCAAAAUUAAAA | 19 | 3981 |
| CCR5-4531 | − | UUUAUGGUUCAAAAUUAAAA | 20 | 3982 |
| CCR5-4532 | − | CUUUAUGGUUCAAAAUUAAAA | 21 | 3983 |
| CCR5-4533 | − | UCUUUAUGGUUCAAAAUUAAAA | 22 | 3984 |
| CCR5-4534 | − | UUCUUUAUGGUUCAAAAUUAAAA | 23 | 3985 |
| CCR5-4535 | − | AUUCUUUAUGGUUCAAAAUUAAAA | 24 | 3986 |
| CCR5-4536 | − | UCUUUUUCCUCCAGACAA | 18 | 3987 |
| CCR5-4537 | − | UUCUUUUUCCUCCAGACAA | 19 | 3988 |
| CCR5-4538 | − | UUUCUUUUUCCUCCAGACAA | 20 | 3989 |
| CCR5-4539 | − | UUUUCUUUUUCCUCCAGACAA | 21 | 3990 |
| CCR5-4540 | − | UUUUUCUUUUUCCUCCAGACAA | 22 | 3991 |
| CCR5-4541 | − | UUUUUUCUUUUUCCUCCAGACAA | 23 | 3992 |
| CCR5-4542 | − | CUUUUUUCUUUUUCCUCCAGACAA | 24 | 3993 |
| CCR5-4543 | − | UGAUCUCUAAGAAGGCAA | 18 | 3994 |
| CCR5-4544 | − | GUGAUCUCUAAGAAGGCAA | 19 | 3995 |
| CCR5-4545 | − | UGUGAUCUCUAAGAAGGCAA | 20 | 3996 |
| CCR5-4546 | − | UUGUGAUCUCUAAGAAGGCAA | 21 | 3997 |
| CCR5-4547 | − | CUUGUGAUCUCUAAGAAGGCAA | 22 | 3998 |
| CCR5-4548 | − | GCUUGUGAUCUCUAAGAAGGCAA | 23 | 3999 |
| CCR5-4549 | − | GGCUUGUGAUCUCUAAGAAGGCAA | 24 | 4000 |
| CCR5-4550 | − | ACUCACAGGGUUUAAUAA | 18 | 4001 |
| CCR5-4551 | − | GACUCACAGGGUUUAAUAA | 19 | 4002 |
| CCR5-4552 | − | AGACUCACAGGGUUUAAUAA | 20 | 4003 |
| CCR5-4553 | − | GAGACUCACAGGGUUUAAUAA | 21 | 4004 |
| CCR5-4554 | − | UGAGACUCACAGGGUUUAAUAA | 22 | 4005 |
| CCR5-4555 | − | UUGAGACUCACAGGGUUUAAUAA | 23 | 4006 |
| CCR5-4556 | − | UUUGAGACUCACAGGGUUUAAUAA | 24 | 4007 |
| CCR5-4557 | − | AGAGCUGAGAAGACAGCA | 18 | 4008 |
| CCR5-4558 | − | CAGAGCUGAGAAGACAGCA | 19 | 4009 |
| CCR5-4559 | − | GCAGAGCUGAGAAGACAGCA | 20 | 4010 |
| CCR5-4560 | − | AGCAGAGCUGAGAAGACAGCA | 21 | 4011 |
| CCR5-4561 | − | CAGCAGAGCUGAGAAGACAGCA | 22 | 4012 |
| CCR5-4562 | − | UCAGCAGAGCUGAGAAGACAGCA | 23 | 4013 |
| CCR5-4563 | − | GUCAGCAGAGCUGAGAAGACAGCA | 24 | 4014 |
| CCR5-4564 | − | CUACAAACACAAACUUCA | 18 | 4015 |
| CCR5-4565 | − | ACUACAAACACAAACUUCA | 19 | 4016 |
| CCR5-4566 | − | AACUACAAACACAAACUUCA | 20 | 4017 |
| CCR5-4567 | − | AAACUACAAACACAAACUUCA | 21 | 4018 |
| CCR5-4568 | − | GAAACUACAAACACAAACUUCA | 22 | 4019 |
| CCR5-4569 | − | AGAAACUACAAACACAAACUUCA | 23 | 4020 |
| CCR5-4570 | − | CAGAAACUACAAACACAAACUUCA | 24 | 4021 |
| CCR5-4571 | − | UUUUUCCUCCAGACAAGA | 18 | 4022 |
| CCR5-4572 | − | CUUUUUCCUCCAGACAAGA | 19 | 4023 |
| CCR5-3072 | − | UCUUUUUCCUCCAGACAAGA | 20 | 4024 |
| CCR5-4573 | − | UUCUUUUUCCUCCAGACAAGA | 21 | 4025 |
| CCR5-4574 | − | UUUCUUUUUCCUCCAGACAAGA | 22 | 4026 |
| CCR5-4575 | − | UUUUCUUUUUCCUCCAGACAAGA | 23 | 4027 |
| CCR5-4576 | − | UUUUUCUUUUUCCUCCAGACAAGA | 24 | 4028 |
| CCR5-4577 | − | UACGUGCCCCCAAUCCUA | 18 | 4029 |
| CCR5-4578 | − | UUACGUGCCCCCAAUCCUA | 19 | 4030 |
| CCR5-4579 | − | AUUACGUGCCCCCAAUCCUA | 20 | 4031 |
| CCR5-4580 | − | AAUUACGUGCCCCCAAUCCUA | 21 | 4032 |
| CCR5-4581 | − | AAAUUACGUGCCCCCAAUCCUA | 22 | 4033 |
| CCR5-4582 | − | AAAAUUACGUGCCCCCAAUCCUA | 23 | 4034 |
| CCR5-4583 | − | CAAAAUUACGUGCCCCCAAUCCUA | 24 | 4035 |
| CCR5-4584 | − | UCUGGACCCAGGAUCUUA | 18 | 4036 |
| CCR5-4585 | − | UUCUGGACCCAGGAUCUUA | 19 | 4037 |
| CCR5-4586 | − | UUUCUGGACCCAGGAUCUUA | 20 | 4038 |
| CCR5-4587 | − | UUUUCUGGACCCAGGAUCUUA | 21 | 4039 |
| CCR5-4588 | − | UUUUUCUGGACCCAGGAUCUUA | 22 | 4040 |
| CCR5-4589 | − | CUUUUUCUGGACCCAGGAUCUUA | 23 | 4041 |
| CCR5-4590 | − | UCUUUUUCUGGACCCAGGAUCUUA | 24 | 4042 |
| CCR5-4591 | − | UUUCUUUUUCCUCCAGAC | 18 | 4043 |
| CCR5-4592 | − | UUUUCUUUUUCCUCCAGAC | 19 | 4044 |
| CCR5-4593 | − | UUUUUCUUUUUCCUCCAGAC | 20 | 4045 |
| CCR5-4594 | − | UUUUUUCUUUUUCCUCCAGAC | 21 | 4046 |
| CCR5-4595 | − | CUUUUUUCUUUUUCCUCCAGAC | 22 | 4047 |
| CCR5-4596 | − | UCUUUUUUCUUUUUCCUCCAGAC | 23 | 4048 |
| CCR5-4597 | − | CUCUUUUUUCUUUUUCCUCCAGAC | 24 | 4049 |
| CCR5-4598 | − | GUCAUCUAUGACCUUCCC | 18 | 4050 |
| CCR5-4599 | − | UGUCAUCUAUGACCUUCCC | 19 | 4051 |
| CCR5-3087 | − | UUGUCAUCUAUGACCUUCCC | 20 | 4052 |
| CCR5-4600 | − | GUUGUCAUCUAUGACCUUCCC | 21 | 4053 |
| CCR5-4601 | − | UGUUGUCAUCUAUGACCUUCCC | 22 | 4054 |
| CCR5-4602 | − | CUGUUGUCAUCUAUGACCUUCCC | 23 | 4055 |
| CCR5-4603 | − | GCUGUUGUCAUCUAUGACCUUCCC | 24 | 4056 |
| CCR5-4604 | − | UGUCAUCUAUGACCUUCC | 18 | 4057 |
| CCR5-4605 | − | UUGUCAUCUAUGACCUUCC | 19 | 4058 |
| CCR5-4606 | − | GUUGUCAUCUAUGACCUUCC | 20 | 4059 |
| CCR5-4607 | − | UGUUGUCAUCUAUGACCUUCC | 21 | 4060 |
| CCR5-4608 | − | CUGUUGUCAUCUAUGACCUUCC | 22 | 4061 |
| CCR5-4609 | − | GCUGUUGUCAUCUAUGACCUUCC | 23 | 4062 |
| CCR5-4610 | − | GGCUGUUGUCAUCUAUGACCUUCC | 24 | 4063 |
| CCR5-4611 | − | UAAGAGAAAAUUCUCAGC | 18 | 4064 |
| CCR5-4612 | − | AUAAGAGAAAAUUCUCAGC | 19 | 4065 |
| CCR5-4613 | − | AAUAAGAGAAAAUUCUCAGC | 20 | 4066 |
| CCR5-4614 | − | UAAUAAGAGAAAAUUCUCAGC | 21 | 4067 |
| CCR5-4615 | − | UUAAUAAGAGAAAAUUCUCAGC | 22 | 4068 |
| CCR5-4616 | − | UUUAAUAAGAGAAAAUUCUCAGC | 23 | 4069 |
| CCR5-4617 | − | GUUUAAUAAGAGAAAAUUCUCAGC | 24 | 4070 |
| CCR5-4618 | − | CUGCCUAGUCUAAGGUGC | 18 | 4071 |
| CCR5-4619 | − | GCUGCCUAGUCUAAGGUGC | 19 | 4072 |
| CCR5-3091 | − | AGCUGCCUAGUCUAAGGUGC | 20 | 4073 |
| CCR5-4620 | − | CAGCUGCCUAGUCUAAGGUGC | 21 | 4074 |
| CCR5-4621 | − | UCAGCUGCCUAGUCUAAGGUGC | 22 | 4075 |
| CCR5-4622 | − | CUCAGCUGCCUAGUCUAAGGUGC | 23 | 4076 |
| CCR5-4623 | − | UCUCAGCUGCCUAGUCUAAGGUGC | 24 | 4077 |
| CCR5-4624 | − | GACAGCAGAGAGCUACUC | 18 | 4078 |
| CCR5-4625 | − | AGACAGCAGAGAGCUACUC | 19 | 4079 |
| CCR5-4626 | − | AAGACAGCAGAGAGCUACUC | 20 | 4080 |
| CCR5-4627 | − | GAAGACAGCAGAGAGCUACUC | 21 | 4081 |
| CCR5-4628 | − | AGAAGACAGCAGAGAGCUACUC | 22 | 4082 |
| CCR5-4629 | − | GAGAAGACAGCAGAGAGCUACUC | 23 | 4083 |
| CCR5-4630 | − | UGAGAAGACAGCAGAGAGCUACUC | 24 | 4084 |
| CCR5-4631 | − | AUUAAAAAUGAGCUUUUC | 18 | 4085 |
| CCR5-4632 | − | AAUUAAAAAUGAGCUUUUC | 19 | 4086 |
| CCR5-4633 | − | AAAUUAAAAAUGAGCUUUUC | 20 | 4087 |
| CCR5-4634 | − | AAAAUUAAAAAUGAGCUUUUC | 21 | 4088 |
| CCR5-4635 | − | CAAAAUUAAAAAUGAGCUUUUC | 22 | 4089 |
| CCR5-4636 | − | UCAAAAUUAAAAAUGAGCUUUUC | 23 | 4090 |
| CCR5-4637 | − | UUCAAAAUUAAAAAUGAGCUUUUC | 24 | 4091 |
| CCR5-4638 | − | CUUUUUCCUCCAGACAAG | 18 | 4092 |
| CCR5-4639 | − | UCUUUUUCCUCCAGACAAG | 19 | 4093 |
| CCR5-3101 | − | UUCUUUUUCCUCCAGACAAG | 20 | 4094 |
| CCR5-4640 | − | UUUCUUUUUCCUCCAGACAAG | 21 | 4095 |
| CCR5-4641 | − | UUUUCUUUUUCCUCCAGACAAG | 22 | 4096 |
| CCR5-4642 | − | UUUUUCUUUUUCCUCCAGACAAG | 23 | 4097 |
| CCR5-4643 | − | UUUUUUCUUUUUCCUCCAGACAAG | 24 | 4098 |
| CCR5-4644 | − | GCAGAGCUGAGAAGACAG | 18 | 4099 |
| CCR5-4645 | − | AGCAGAGCUGAGAAGACAG | 19 | 4100 |
| CCR5-4646 | − | CAGCAGAGCUGAGAAGACAG | 20 | 4101 |
| CCR5-4647 | − | UCAGCAGAGCUGAGAAGACAG | 21 | 4102 |
| CCR5-4648 | − | GUCAGCAGAGCUGAGAAGACAG | 22 | 4103 |
| CCR5-4649 | − | UGUCAGCAGAGCUGAGAAGACAG | 23 | 4104 |
| CCR5-4650 | − | UUGUCAGCAGAGCUGAGAAGACAG | 24 | 4105 |
| CCR5-4651 | − | AAUUCUCAGCUAGAGCAG | 18 | 4106 |
| CCR5-4652 | − | AAAUUCUCAGCUAGAGCAG | 19 | 4107 |
| CCR5-4653 | − | AAAAUUCUCAGCUAGAGCAG | 20 | 4108 |
| CCR5-4654 | − | GAAAAUUCUCAGCUAGAGCAG | 21 | 4109 |
| CCR5-4655 | − | AGAAAAUUCUCAGCUAGAGCAG | 22 | 4110 |
| CCR5-4656 | − | GAGAAAAUUCUCAGCUAGAGCAG | 23 | 4111 |
| CCR5-4657 | − | AGAGAAAAUUCUCAGCUAGAGCAG | 24 | 4112 |
| CCR5-4658 | − | AUUCAUCUGUGGUGGCAG | 18 | 4113 |
| CCR5-4659 | − | CAUUCAUCUGUGGUGGCAG | 19 | 4114 |
| CCR5-4660 | − | ACAUUCAUCUGUGGUGGCAG | 20 | 4115 |
| CCR5-4661 | − | GACAUUCAUCUGUGGUGGCAG | 21 | 4116 |
| CCR5-4662 | − | UGACAUUCAUCUGUGGUGGCAG | 22 | 4117 |
| CCR5-4663 | − | AUGACAUUCAUCUGUGGUGGCAG | 23 | 4118 |
| CCR5-4664 | − | CAUGACAUUCAUCUGUGGUGGCAG | 24 | 4119 |
| CCR5-4665 | − | AAUCUCAAGUAUUGUCAG | 18 | 4120 |
| CCR5-4666 | − | AAAUCUCAAGUAUUGUCAG | 19 | 4121 |
| CCR5-4667 | − | AAAAUCUCAAGUAUUGUCAG | 20 | 4122 |
| CCR5-4668 | − | GAAAAUCUCAAGUAUUGUCAG | 21 | 4123 |
| CCR5-4669 | − | UGAAAAUCUCAAGUAUUGUCAG | 22 | 4124 |
| CCR5-4670 | − | CUGAAAAUCUCAAGUAUUGUCAG | 23 | 4125 |
| CCR5-4671 | − | UCUGAAAAUCUCAAGUAUUGUCAG | 24 | 4126 |
| CCR5-4672 | − | CAAGUAUUGUCAGCAGAG | 18 | 4127 |
| CCR5-4673 | − | UCAAGUAUUGUCAGCAGAG | 19 | 4128 |
| CCR5-4674 | − | CUCAAGUAUUGUCAGCAGAG | 20 | 4129 |
| CCR5-4675 | − | UCUCAAGUAUUGUCAGCAGAG | 21 | 4130 |
| CCR5-4676 | − | AUCUCAAGUAUUGUCAGCAGAG | 22 | 4131 |
| CCR5-4677 | − | AAUCUCAAGUAUUGUCAGCAGAG | 23 | 4132 |
| CCR5-4678 | − | AAAUCUCAAGUAUUGUCAGCAGAG | 24 | 4133 |
| CCR5-4679 | − | CUGGACCCAGGAUCUUAG | 18 | 4134 |
| CCR5-4680 | − | UCUGGACCCAGGAUCUUAG | 19 | 4135 |
| CCR5-3106 | − | UUCUGGACCCAGGAUCUUAG | 20 | 4136 |
| CCR5-4681 | − | UUUCUGGACCCAGGAUCUUAG | 21 | 4137 |
| CCR5-4682 | − | UUUUCUGGACCCAGGAUCUUAG | 22 | 4138 |
| CCR5-4683 | − | UUUUUCUGGACCCAGGAUCUUAG | 23 | 4139 |
| CCR5-4684 | − | CUUUUUCUGGACCCAGGAUCUUAG | 24 | 4140 |
| CCR5-4685 | − | UUAACUAUGGGCUCACGG | 18 | 4141 |
| CCR5-4686 | − | UUUAACUAUGGGCUCACGG | 19 | 4142 |
| CCR5-4687 | − | UUUUAACUAUGGGCUCACGG | 20 | 4143 |
| CCR5-4688 | − | GUUUUAACUAUGGGCUCACGG | 21 | 4144 |
| CCR5-4689 | − | AGUUUUAACUAUGGGCUCACGG | 22 | 4145 |
| CCR5-4690 | − | GAGUUUUAACUAUGGGCUCACGG | 23 | 4146 |
| CCR5-4691 | − | AGAGUUUUAACUAUGGGCUCACGG | 24 | 4147 |
| CCR5-4692 | − | GCUGCCUAGUCUAAGGUG | 18 | 4148 |
| CCR5-4693 | − | AGCUGCCUAGUCUAAGGUG | 19 | 4149 |
| CCR5-4694 | − | CAGCUGCCUAGUCUAAGGUG | 20 | 4150 |
| CCR5-4695 | − | UCAGCUGCCUAGUCUAAGGUG | 21 | 4151 |
| CCR5-4696 | − | CUCAGCUGCCUAGUCUAAGGUG | 22 | 4152 |
| CCR5-4697 | − | UCUCAGCUGCCUAGUCUAAGGUG | 23 | 4153 |
| CCR5-4698 | − | CUCUCAGCUGCCUAGUCUAAGGUG | 24 | 4154 |
| CCR5-4699 | − | ACAAACUUCACAGAAAAU | 18 | 4155 |
| CCR5-4700 | − | CACAAACUUCACAGAAAAU | 19 | 4156 |
| CCR5-4701 | − | ACACAAACUUCACAGAAAAU | 20 | 4157 |
| CCR5-4702 | − | AACACAAACUUCACAGAAAAU | 21 | 4158 |
| CCR5-4703 | − | AAACACAAACUUCACAGAAAAU | 22 | 4159 |
| CCR5-4704 | − | CAAACACAAACUUCACAGAAAAU | 23 | 4160 |
| CCR5-4705 | − | ACAAACACAAACUUCACAGAAAAU | 24 | 4161 |
| CCR5-4706 | − | AGACUCACAGGGUUUAAU | 18 | 4162 |
| CCR5-4707 | − | GAGACUCACAGGGUUUAAU | 19 | 4163 |
| CCR5-4708 | − | UGAGACUCACAGGGUUUAAU | 20 | 4164 |
| CCR5-4709 | − | UUGAGACUCACAGGGUUUAAU | 21 | 4165 |
| CCR5-4710 | − | UUUGAGACUCACAGGGUUUAAU | 22 | 4166 |
| CCR5-4711 | − | GUUUGAGACUCACAGGGUUUAAU | 23 | 4167 |
| CCR5-4712 | − | AGUUUGAGACUCACAGGGUUUAAU | 24 | 4168 |
| CCR5-4713 | − | CUUGGCGGUUGGUGACAU | 18 | 4169 |
| CCR5-4714 | − | UCUUGGCGGUUGGUGACAU | 19 | 4170 |
| CCR5-4715 | − | CUCUUGGCGGUUGGUGACAU | 20 | 4171 |
| CCR5-4716 | − | UCUCUUGGCGGUUGGUGACAU | 21 | 4172 |
| CCR5-4717 | − | CUCUCUUGGCGGUUGGUGACAU | 22 | 4173 |
| CCR5-4718 | − | GCUCUCUUGGCGGUUGGUGACAU | 23 | 4174 |
| CCR5-4719 | − | AGCUCUCUUGGCGGUUGGUGACAU | 24 | 4175 |
| CCR5-4720 | − | UAAUCUAUCUGAAGCUAU | 18 | 4176 |
| CCR5-4721 | − | AUAAUCUAUCUGAAGCUAU | 19 | 4177 |
| CCR5-4722 | − | UAUAAUCUAUCUGAAGCUAU | 20 | 4178 |
| CCR5-4723 | − | AUAUAAUCUAUCUGAAGCUAU | 21 | 4179 |
| CCR5-4724 | − | GAUAUAAUCUAUCUGAAGCUAU | 22 | 4180 |
| CCR5-4725 | − | AGAUAUAAUCUAUCUGAAGCUAU | 23 | 4181 |
| CCR5-4726 | − | CAGAUAUAAUCUAUCUGAAGCUAU | 24 | 4182 |
| CCR5-4727 | − | ACUCCAGAUAUAAUCUAU | 18 | 4183 |
| CCR5-4728 | − | CACUCCAGAUAUAAUCUAU | 19 | 4184 |
| CCR5-4729 | − | UCACUCCAGAUAUAAUCUAU | 20 | 4185 |
| CCR5-4730 | − | UUCACUCCAGAUAUAAUCUAU | 21 | 4186 |
| CCR5-4731 | − | CUUCACUCCAGAUAUAAUCUAU | 22 | 4187 |
| CCR5-4732 | − | UCUUCACUCCAGAUAUAAUCUAU | 23 | 4188 |
| CCR5-4733 | − | UUCUUCACUCCAGAUAUAAUCUAU | 24 | 4189 |
| CCR5-4734 | − | AAACCAGUAAGCAUUUAU | 18 | 4190 |
| CCR5-4735 | − | CAAACCAGUAAGCAUUUAU | 19 | 4191 |
| CCR5-4736 | − | UCAAACCAGUAAGCAUUUAU | 20 | 4192 |
| CCR5-4737 | − | UUCAAACCAGUAAGCAUUUAU | 21 | 4193 |
| CCR5-4738 | − | CUUCAAACCAGUAAGCAUUUAU | 22 | 4194 |
| CCR5-4739 | − | CCUUCAAACCAGUAAGCAUUUAU | 23 | 4195 |
| CCR5-4740 | − | CCCUUCAAACCAGUAAGCAUUUAU | 24 | 4196 |
| CCR5-4741 | − | CUCUUAAUUGUGGCAACU | 18 | 4197 |
| CCR5-4742 | − | ACUCUUAAUUGUGGCAACU | 19 | 4198 |
| CCR5-4743 | − | AACUCUUAAUUGUGGCAACU | 20 | 4199 |
| CCR5-4744 | − | CAACUCUUAAUUGUGGCAACU | 21 | 4200 |
| CCR5-4745 | − | ACAACUCUUAAUUGUGGCAACU | 22 | 4201 |
| CCR5-4746 | − | GACAACUCUUAAUUGUGGCAACU | 23 | 4202 |
| CCR5-4747 | − | UGACAACUCUUAAUUGUGGCAACU | 24 | 4203 |
| CCR5-4748 | − | GUCUAAAGAGUUUUAACU | 18 | 4204 |
| CCR5-4749 | − | UGUCUAAAGAGUUUUAACU | 19 | 4205 |
| CCR5-4750 | − | UUGUCUAAAGAGUUUUAACU | 20 | 4206 |
| CCR5-4751 | − | GUUGUCUAAAGAGUUUUAACU | 21 | 4207 |
| CCR5-4752 | − | UGUUGUCUAAAGAGUUUUAACU | 22 | 4208 |
| CCR5-4753 | − | CUGUUGUCUAAAGAGUUUUAACU | 23 | 4209 |
| CCR5-4754 | − | CCUGUUGUCUAAAGAGUUUUAACU | 24 | 4210 |
| CCR5-4755 | − | CGAGCCACAAGAUGCCCU | 18 | 4211 |
| CCR5-4756 | − | CCGAGCCACAAGAUGCCCU | 19 | 4212 |
| CCR5-4757 | − | CCCGAGCCACAAGAUGCCCU | 20 | 4213 |
| CCR5-4758 | − | UCCCGAGCCACAAGAUGCCCU | 21 | 4214 |
| CCR5-4759 | − | CUCCCGAGCCACAAGAUGCCCU | 22 | 4215 |
| CCR5-4760 | − | ACUCCCGAGCCACAAGAUGCCCU | 23 | 4216 |
| CCR5-4761 | − | UACUCCCGAGCCACAAGAUGCCCU | 24 | 4217 |
| CCR5-4762 | − | UAUAAUCUAUCUGAAGCU | 18 | 4218 |
| CCR5-4763 | − | AUAUAAUCUAUCUGAAGCU | 19 | 4219 |
| CCR5-4764 | − | GAUAUAAUCUAUCUGAAGCU | 20 | 4220 |
| CCR5-4765 | − | AGAUAUAAUCUAUCUGAAGCU | 21 | 4221 |
| CCR5-4766 | − | CAGAUAUAAUCUAUCUGAAGCU | 22 | 4222 |
| CCR5-4767 | − | CCAGAUAUAAUCUAUCUGAAGCU | 23 | 4223 |
| CCR5-4768 | − | UCCAGAUAUAAUCUAUCUGAAGCU | 24 | 4224 |
| CCR5-4769 | − | AGUAUUGUCAGCAGAGCU | 18 | 4225 |
| CCR5-4770 | − | AAGUAUUGUCAGCAGAGCU | 19 | 4226 |
| CCR5-4771 | − | CAAGUAUUGUCAGCAGAGCU | 20 | 4227 |
| CCR5-4772 | − | UCAAGUAUUGUCAGCAGAGCU | 21 | 4228 |
| CCR5-4773 | − | CUCAAGUAUUGUCAGCAGAGCU | 22 | 4229 |
| CCR5-4774 | − | UCUCAAGUAUUGUCAGCAGAGCU | 23 | 4230 |
| CCR5-4775 | − | AUCUCAAGUAUUGUCAGCAGAGCU | 24 | 4231 |
| CCR5-4776 | − | CUUUGGCUUGUGAUCUCU | 18 | 4232 |
| CCR5-4777 | − | GCUUUGGCUUGUGAUCUCU | 19 | 4233 |
| CCR5-4778 | − | AGCUUUGGCUUGUGAUCUCU | 20 | 4234 |
| CCR5-4779 | − | AAGCUUUGGCUUGUGAUCUCU | 21 | 4235 |
| CCR5-4780 | − | AAAGCUUUGGCUUGUGAUCUCU | 22 | 4236 |
| CCR5-4781 | − | AAAAGCUUUGGCUUGUGAUCUCU | 23 | 4237 |
| CCR5-4782 | − | AAAAAGCUUUGGCUUGUGAUCUCU | 24 | 4238 |
| CCR5-4783 | − | UUAAAAAUGAGCUUUUCU | 18 | 4239 |
| CCR5-4784 | − | AUUAAAAAUGAGCUUUUCU | 19 | 4240 |
| CCR5-3134 | − | AAUUAAAAAUGAGCUUUUCU | 20 | 4241 |
| CCR5-4785 | − | AAAUUAAAAAUGAGCUUUUCU | 21 | 4242 |
| CCR5-4786 | − | AAAAUUAAAAAUGAGCUUUUCU | 22 | 4243 |
| CCR5-4787 | − | CAAAAUUAAAAAUGAGCUUUUCU | 23 | 4244 |
| CCR5-4788 | − | UCAAAAUUAAAAAUGAGCUUUUCU | 24 | 4245 |
| CCR5-4789 | − | GUCUAAGGUGCAGGGAGU | 18 | 4246 |
| CCR5-4790 | − | AGUCUAAGGUGCAGGGAGU | 19 | 4247 |
| CCR5-4791 | − | UAGUCUAAGGUGCAGGGAGU | 20 | 4248 |
| CCR5-4792 | − | CUAGUCUAAGGUGCAGGGAGU | 21 | 4249 |
| CCR5-4793 | − | CCUAGUCUAAGGUGCAGGGAGU | 22 | 4250 |
| CCR5-4794 | − | GCCUAGUCUAAGGUGCAGGGAGU | 23 | 4251 |
| CCR5-4795 | − | UGCCUAGUCUAAGGUGCAGGGAGU | 24 | 4252 |
| CCR5-4796 | − | UCAAACCAGUAAGCAUUU | 18 | 4253 |
| CCR5-4797 | − | UUCAAACCAGUAAGCAUUU | 19 | 4254 |
| CCR5-4798 | − | CUUCAAACCAGUAAGCAUUU | 20 | 4255 |
| CCR5-4799 | − | CCUUCAAACCAGUAAGCAUUU | 21 | 4256 |
| CCR5-4800 | − | CCCUUCAAACCAGUAAGCAUUU | 22 | 4257 |
| CCR5-4801 | − | GCCCUUCAAACCAGUAAGCAUUU | 23 | 4258 |
| CCR5-4802 | − | UGCCCUUCAAACCAGUAAGCAUUU | 24 | 4259 |
| CCR5-4803 | − | CAGGUUUCCCAUCUUUUU | 18 | 4260 |
| CCR5-4804 | − | ACAGGUUUCCCAUCUUUUU | 19 | 4261 |
| CCR5-4805 | − | AACAGGUUUCCCAUCUUUUU | 20 | 4262 |
| CCR5-4806 | − | AAACAGGUUUCCCAUCUUUUU | 21 | 4263 |
| CCR5-4807 | − | UAAACAGGUUUCCCAUCUUUUU | 22 | 4264 |
| CCR5-4808 | − | CUAAACAGGUUUCCCAUCUUUUU | 23 | 4265 |
| CCR5-4809 | − | GCUAAACAGGUUUCCCAUCUUUUU | 24 | 4266 |

Table 7A provides exemplary targeting domains for knocking down the CCR5 gene selected according to the first tier parameters. The targeting domains bind within 500 bp (e.g., upstream or downstream) of a transcription start site (TSS) and have a high level of orthogonality. It is contemplated herein that in an embodiment the targeting domain hybridizes to the target domain through complementary base pairing. Any of the targeting domains in the table can be used with a N. meningitidis eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 7A
1st Tier

| | | | Target | |
|---|---|---|---|---|
| gRNA | DNA | | Site | SEQ ID |
| Name | Strand | Targeting Domain | Length | NO |
| CCR5-4810 | − | AUCCUUACCUCUCAAAA | 17 | 4267 |
| CCR5-4811 | + | CUAAAAGGUUAAGAAAA | 17 | 4268 |
| CCR5-4812 | − | AGCUGCUUGGCCUGUUA | 17 | 4269 |
| CCR5-4813 | + | AUUACUAUCCAAGAAGC | 17 | 4270 |
| CCR5-4814 | − | GUGAUCUUGUACAAAUC | 17 | 4271 |
| CCR5-4815 | − | CCGGUAAGUAACCUCUC | 17 | 4272 |
| CCR5-4816 | + | AUUUACGGGCUUUUCUC | 17 | 4273 |
| CCR5-4817 | − | AGACCAGAGAUCUAUUC | 17 | 4274 |
| CCR5-4818 | + | GUUCUCCUUAGCAGAAG | 17 | 4275 |
| CCR5-4819 | + | AUCUUUCUUUUGAGAGG | 17 | 4276 |
| CCR5-4820 | − | UUUUAUACUGUCUAUAU | 17 | 4277 |
| CCR5-4821 | − | UUCGCCUUCAAUACACU | 17 | 4278 |
| CCR5-4822 | + | UGACCCUUUCCUUAUCU | 17 | 4279 |
| CCR5-4823 | − | CUACUUUUAUACUGUCU | 17 | 4280 |
| CCR5-4824 | − | UAAAAAGAAGAACUGUU | 17 | 4281 |
| CCR5-4825 | + | GGUCUGAAGGUUUAUUU | 17 | 4282 |
| CCR5-4826 | − | ACAAUCCUUACCUCUCAAAA | 20 | 4283 |
| CCR5-4827 | + | AGGCUAAAAGGUUAAGAAAA | 20 | 4284 |
| CCR5-4828 | − | UACAUUUAAAGUUGGUUUAA | 20 | 4285 |
| CCR5-4829 | − | CUCAGCUGCUUGGCCUGUUA | 20 | 4286 |
| CCR5-4830 | + | GAAAUUACUAUCCAAGAAGC | 20 | 4287 |
| CCR5-4831 | − | CCUGUGAUCUUGUACAAAUC | 20 | 4288 |
| CCR5-4832 | − | UCCCCGGUAAGUAACCUCUC | 20 | 4289 |
| CCR5-4833 | + | UUUAUUUACGGGCUUUUCUC | 20 | 4290 |
| CCR5-4834 | − | UUCAGACCAGAGAUCUAUUC | 20 | 4291 |
| CCR5-4835 | + | UUAGUUCUCCUUAGCAGAAG | 20 | 4292 |
| CCR5-3491 | + | GAACAGUUCUUCUUUUUAAG | 20 | 4293 |
| CCR5-4836 | + | CAAAUCUUUCUUUUGAGAGG | 20 | 4294 |
| CCR5-4837 | − | UACUUUUAUACUGUCUAUAU | 20 | 4295 |
| CCR5-4838 | − | CUUUUCGCCUUCAAUACACU | 20 | 4296 |
| CCR5-4839 | + | CUGUGACCCUUUCCUUAUCU | 20 | 4297 |
| CCR5-4840 | − | UUCCUACUUUUAUACUGUCU | 20 | 4298 |
| CCR5-4841 | + | CCUUAGCAGAAGAUAAGAUU | 20 | 4299 |
| CCR5-4842 | − | ACUUAAAAAGAAGAACUGUU | 20 | 4300 |
| CCR5-3668 | + | UCUGGUCUGAAGGUUUAUUU | 20 | 4301 |

Table 7B provides exemplary targeting domains for knocking down the CCR5 gene selected according to the second tier parameters. The targeting domains bind within 500 bp (e.g., upstream or downstream) of a transcription start site (TSS). It is contemplated herein that in an embodiment the targeting domain hybridizes to the target domain through complementary base pairing. Any of the targeting domains in the table can be used with a N. meningitidis eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 7B
2nd Tier

| | | | Target | |
|---|---|---|---|---|
| gRNA | DNA | | Site | SEQ ID |
| Name | Strand | Targeting Domain | Length | NO |
| CCR5-4843 | − | AACAUCAAAGAUACAAA | 17 | 4302 |
| CCR5-4844 | − | AUUUAAAGUUGGUUUAA | 17 | 4303 |
| CCR5-4845 | + | UGAUUUGUACAAGAUCA | 17 | 4304 |
| CCR5-4846 | + | CAGUUCUUCUUUUUAAG | 17 | 4305 |
| CCR5-4847 | − | AUUUCUUUUACUAAAAU | 17 | 4306 |
| CCR5-4848 | − | UAUUCUUUAUAUUUUCU | 17 | 4307 |
| CCR5-4849 | + | UAGCAGAAGAUAAGAUU | 17 | 4308 |
| CCR5-4850 | − | UAUAACAUCAAAGAUACAAA | 20 | 4309 |
| CCR5-3386 | + | AAAUGAUUUGUACAAGAUCA | 20 | 4310 |
| CCR5-3978 | − | GUAAUUUCUUUUACUAAAAU | 20 | 4311 |
| CCR5-4851 | − | CUUUAUUCUUUAUAUUUUCU | 20 | 4312 |

Table 7C provides exemplary targeting domains for knocking down the CCR5 gene selected according to the third tier parameters. Within the additional 500 bp (e.g., upstream or downstream) of a transcription start site (TSS), e.g., extending to 1 kb upstream and downstream of a TSS. It is contemplated herein that in an embodiment the targeting domain hybridizes to the target domain through complementary base pairing. Any of the targeting domains in the table can be used with a N. meningitidis eiCas9 molecule or eiCas9 fusion protein (e.g., an eiCas9 fused to a transcription repressor domain) to alter the CCR5 gene (e.g., reduce or eliminate CCR5 gene expression, CCR5 protein function, or the level of CCR5 protein). One or more gRNAs may be used to target an eiCas9 to the promoter region of the CCR5 gene.

TABLE 7C
3rd Tier

| | | | Target | |
|---|---|---|---|---|
| gRNA | DNA | | Site | SEQ ID |
| Name | Strand | Targeting Domain | Length | NO |
| CCR5-4852 | − | AUGGUUCAAAAUUAAAA | 17 | 4313 |
| CCR5-4853 | + | AUGUCACCAACCGCCAA | 17 | 4314 |
| CCR5-4854 | + | AAUUUCUCAUAGCUUCA | 17 | 4315 |
| CCR5-4855 | − | ACCUUGGCUCUAGAAUA | 17 | 4316 |
| CCR5-4856 | + | AGCUCUGCUGACAAUAC | 17 | 4317 |
| CCR5-4857 | − | GCUCUAGAAUAAAAAGC | 17 | 4318 |
| CCR5-4858 | + | UCUUAGAGAUCACAAGC | 17 | 4319 |
| CCR5-3022 | − | UGGACCCAGGAUCUUAG | 17 | 4320 |
| CCR5-4859 | − | AAACUUCACAGAAAAUG | 17 | 4321 |
| CCR5-4860 | − | UGCCAGAUACAUAGGUG | 17 | 4322 |
| CCR5-4861 | + | AUAGUGUGAGUCCUCAU | 17 | 4323 |
| CCR5-4862 | − | GAGCCACAAGAUGCCCU | 17 | 4324 |
| CCR5-4863 | + | UCAUGUGGAAAAUUUCU | 17 | 4325 |
| CCR5-3052 | − | UAAAAAUGAGCUUUUCU | 17 | 4326 |
| CCR5-4864 | + | AUUAAUUUUGACCAUUU | 17 | 4327 |
| CCR5-4531 | − | UUUAUGGUUCAAAAUUAAAA | 20 | 4328 |
| CCR5-4231 | + | CAGAUGUCACCAACCGCCAA | 20 | 4329 |
| CCR5-4865 | + | GAAAAUUUCUCAUAGCUUCA | 20 | 4330 |
| CCR5-4866 | − | GUGACCUUGGCUCUAGAAUA | 20 | 4331 |
| CCR5-4306 | + | CUCAGCUCUGCUGACAAUAC | 20 | 4332 |
| CCR5-4867 | − | UUGGCUCUAGAAUAAAAAGC | 20 | 4333 |
| CCR5-4868 | + | CCUUCUUAGAGAUCACAAGC | 20 | 4334 |
| CCR5-3106 | − | UUCUGGACCCAGGAUCUUAG | 20 | 4335 |
| CCR5-4869 | − | CACAAACUUCACAGAAAAUG | 20 | 4336 |
| CCR5-4870 | − | CUAUGCCAGAUACAUAGGUG | 20 | 4337 |
| CCR5-4871 | + | GGCAUAGUGUGAGUCCUCAU | 20 | 4338 |
| CCR5-4757 | − | CCCGAGCCACAAGAUGCCCU | 20 | 4339 |
| CCR5-4872 | + | AUGUCAUGUGGAAAAUUUCU | 20 | 4340 |
| CCR5-3134 | − | AAUUAAAAAUGAGCUUUUCU | 20 | 4341 |
| CCR5-4873 | + | AAUAUUAAUUUUGACCAUUU | 20 | 4342 |

### III. Cas9 Molecules

Cas9 molecules of a variety of species can be used in the methods and compositions described herein. While the S. pyogenes, S. aureus, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes and S. thermophilus Cas9 molecules, Cas9 molecules from the other species can replace them, e.g., Staphylococcus aureus and Neisseria meningitides Cas9 molecules. Additional Cas9 species include: Acidovorax avenae, Actinobacillus pleuropneumonias, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

A Cas9 molecule, or Cas9 polypeptide, as that term is used herein, refers to a molecule or polypeptide that can interact with a guide RNA (gRNA) molecule and, in concert with the gRNA molecule, home or localizes to a site which comprises a target domain and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule or a sequence of Table 8.

Cas9 Domains

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

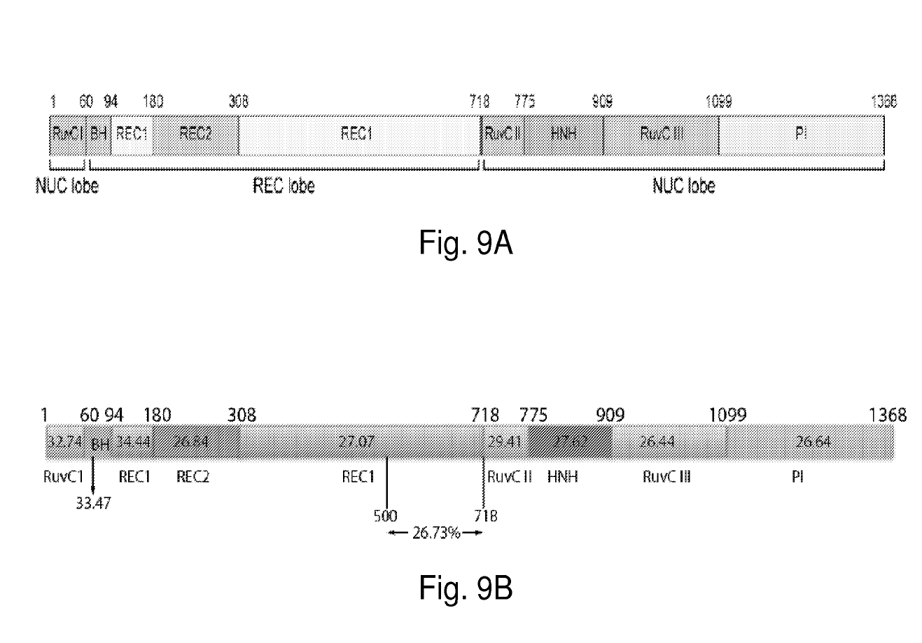

A naturally occurring Cas9 molecule comprises two lobes: a recognition (REC) lobe and a nuclease (NUC) lobe; each of which further comprises domains described herein. FIGS. 9A-9B provide a schematic of the organization of important Cas9 domains in the primary structure. The domain nomenclature and the numbering of the amino acid residues encompassed by each domain used throughout this disclosure is as described in Nishimasu et al. The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long a helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvCI, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

A RuvC-Like Domain and an HNH-Like Domain

In an embodiment, a Cas9 molecule or Cas9 polypeptide comprises an HNH-like domain and a RuvC-like domain. In an embodiment, cleavage activity is dependent on a RuvC-like domain and an HNH-like domain. A Cas9 molecule or Cas9 polypeptide, e.g., an eaCas9 molecule or eaCas9 polypeptide, can comprise one or more of the following domains: a RuvC-like domain and an HNH-like domain. In an embodiment, a Cas9 molecule or Cas9 polypeptide is an eaCas9 molecule or eaCas9 polypeptide and the eaCas9 molecule or eaCas9 polypeptide comprises a RuvC-like domain, e.g., a RuvC-like domain described below, and/or an HNH-like domain, e.g., an HNH-like domain described below.

RuvC-Like Domains

In an embodiment, a RuvC-like domain cleaves, a single strand, e.g., the non-complementary strand of the target nucleic acid molecule. The Cas9 molecule or Cas9 polypeptide can include more than one RuvC-like domain (e.g., one, two, three or more RuvC-like domains). In an embodiment, a RuvC-like domain is at least 5, 6, 7, 8 amino acids in length but not more than 20, 19, 18, 17, 16 or 15 amino acids in length. In an embodiment, the Cas9 molecule or Cas9 polypeptide comprises an N-terminal RuvC-like domain of about 10 to 20 amino acids, e.g., about 15 amino acids in length.

N-Terminal RuvC-Like Domains

Some naturally occurring Cas9 molecules comprise more than one RuvC-like domain with cleavage being dependent on the N-terminal RuvC-like domain. Accordingly, Cas9 molecules or Cas9 polypeptide can comprise an N-terminal RuvC-like domain. Exemplary N-terminal RuvC-like domains are described below.

In an embodiment, an eaCas9 molecule or eaCas9 polypeptide comprises an N-terminal RuvC-like domain comprising an amino acid sequence of formula I:

(SEQ ID NO: 8)

D-X1-G-X2-X3-X4-X5-G-X6-X7-X8-X9,

wherein,

X1 is selected from I, V, M, L and T (e.g., selected from I, V, and L);

X2 is selected from T, I, V, S, N, Y, E and L (e.g., selected from T, V, and I);

X3 is selected from N, S, G, A, D, T, R, M and F (e.g., A or N);

X4 is selected from S, Y, N and F (e.g., S);

X5 is selected from V, I, L, C, T and F (e.g., selected from V, I and L);

X6 is selected from W, F, V, Y, S and L (e.g., W);

X7 is selected from A, S, C, V and G (e.g., selected from A and S);

X8 is selected from V, I, L, A, M and H (e.g., selected from V, I, M and L); and

X9 is selected from any amino acid or is absent (e.g., selected from T, V, I, L, Δ, F, S, A, Y, M and R, or, e.g., selected from T, V, I, L and Δ).

In an embodiment, the N-terminal RuvC-like domain differs from a sequence of SEQ ID NO:8, by as many as 1 but no more than 2, 3, 4, or 5 residues.

In embodiment, the N-terminal RuvC-like domain is cleavage competent.

In embodiment, the N-terminal RuvC-like domain is cleavage incompetent.

In an embodiment, a eaCas9 molecule or eaCas9 polypeptide comprises an N-terminal RuvC-like domain comprising an amino acid sequence of formula II:

(SEQ ID NO: 9)

D-X1-G-X2-X3-S-X5-G-X6-X7-X8-X9,,

wherein

X1 is selected from I, V, M, L and T (e.g., selected from I, V, and L);

X2 is selected from T, I, V, S, N, Y, E and L (e.g., selected from T, V, and I);

X3 is selected from N, S, G, A, D, T, R, M and F (e.g., A or N);

X5 is selected from V, I, L, C, T and F (e.g., selected from V, I and L);

X6 is selected from W, F, V, Y, S and L (e.g., W);

X7 is selected from A, S, C, V and G (e.g., selected from A and S);

X8 is selected from V, I, L, A, M and H (e.g., selected from V, I, M and L); and

X9 is selected from any amino acid or is absent (e.g., selected from T, V, I, L, Δ, F, S, A, Y, M and R or selected from e.g., T, V, I, L and Δ).

In an embodiment, the N-terminal RuvC-like domain differs from a sequence of SEQ ID NO:9 by as many as 1 but no more than 2, 3, 4, or 5 residues.

In an embodiment, the N-terminal RuvC-like domain comprises an amino acid sequence of formula III:

(SEQ ID NO: 10)

D-I-G-X2-X3-S-V-G-W-A-X8-X9,

wherein

X2 is selected from T, I, V, S, N, Y, E and L (e.g., selected from T, V, and I);

X3 is selected from N, S, G, A, D, T, R, M and F (e.g., A or N);

X8 is selected from V, I, L, A, M and H (e.g., selected from V, I, M and L); and

X9 is selected from any amino acid or is absent (e.g., selected from T, V, I, L, Δ, F, S, A, Y, M and R or selected from e.g., T, V, I, L and Δ).

In an embodiment, the N-terminal RuvC-like domain differs from a sequence of SEQ ID NO:10 by as many as 1 but no more than, 2, 3, 4, or 5 residues.

In an embodiment, the N-terminal RuvC-like domain comprises an amino acid sequence of formula III:

(SEQ ID NO: 11)

D-I-G-T-N-S-V-G-W-A-V-X,

wherein

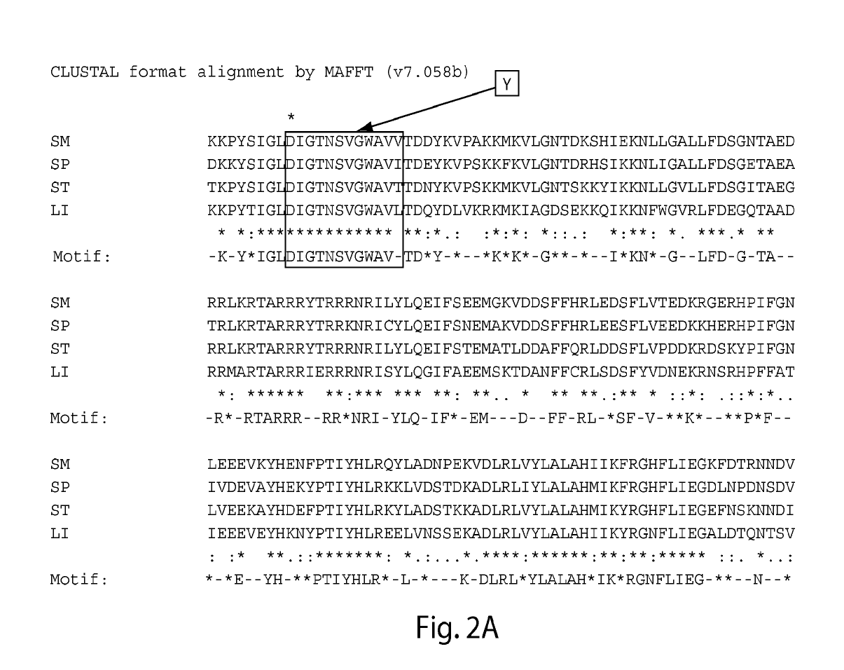

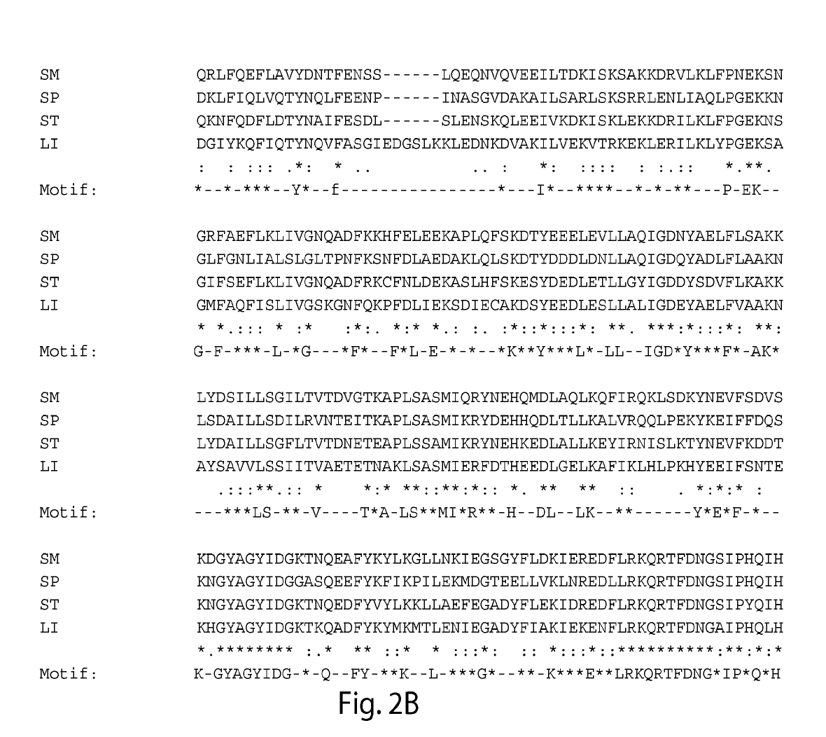

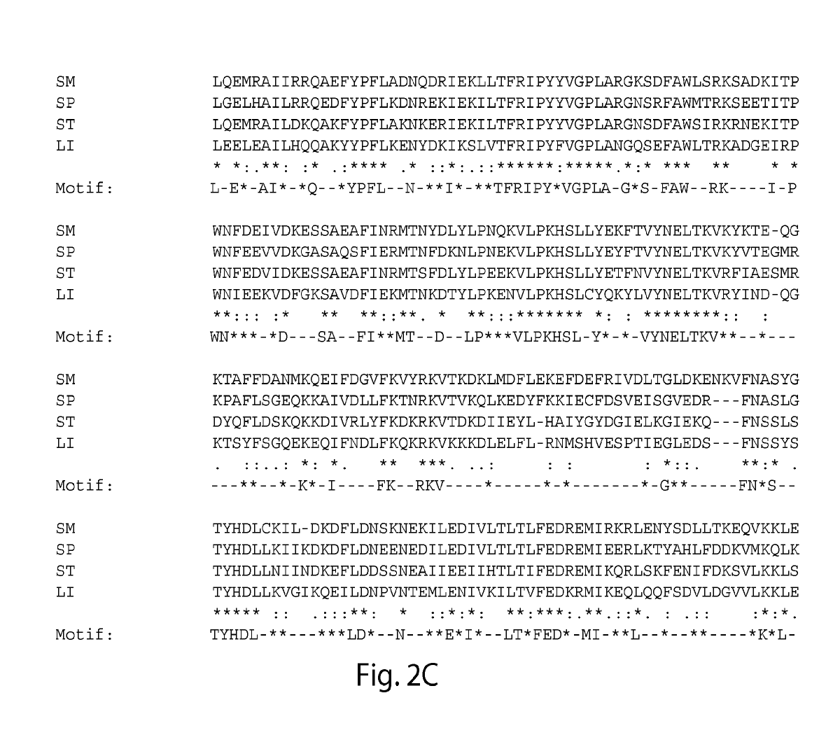

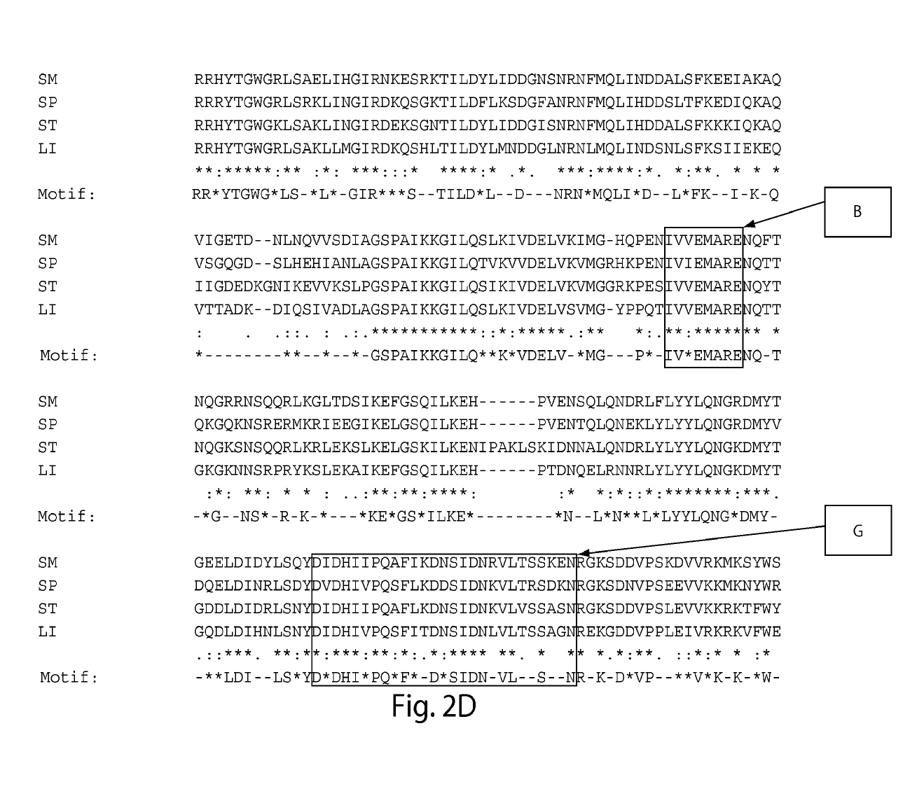

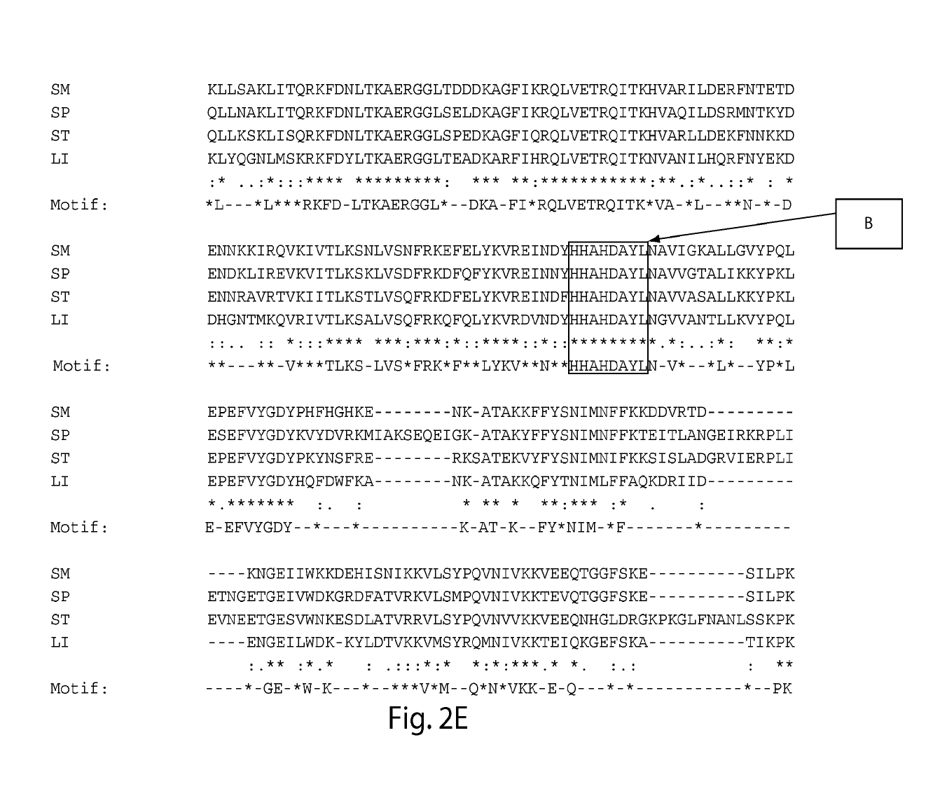

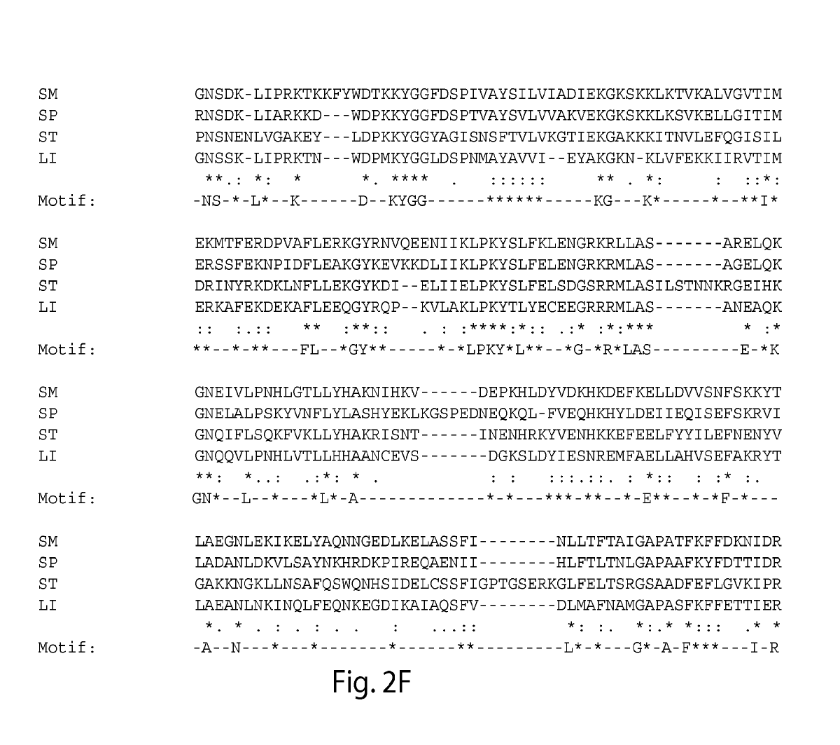

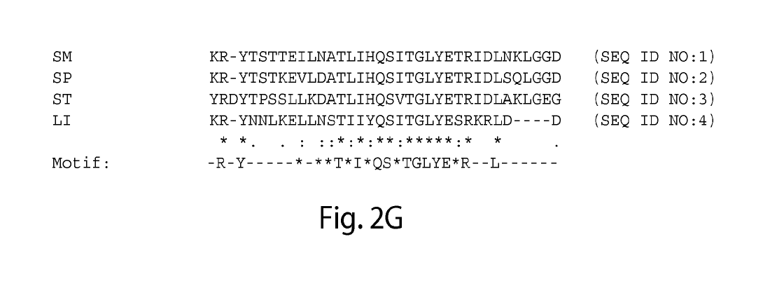

X is a non-polar alkyl amino acid or a hydroxyl amino acid, e.g., X is selected from V, I, L and T (e.g., the eaCas9 molecule can comprise an N-terminal RuvC-like domain shown in FIGS. 2A-2G (is depicted as Y)).

In an embodiment, the N-terminal RuvC-like domain differs from a sequence of SEQ ID NO:11 by as many as 1 but no more than, 2, 3, 4, or 5 residues.

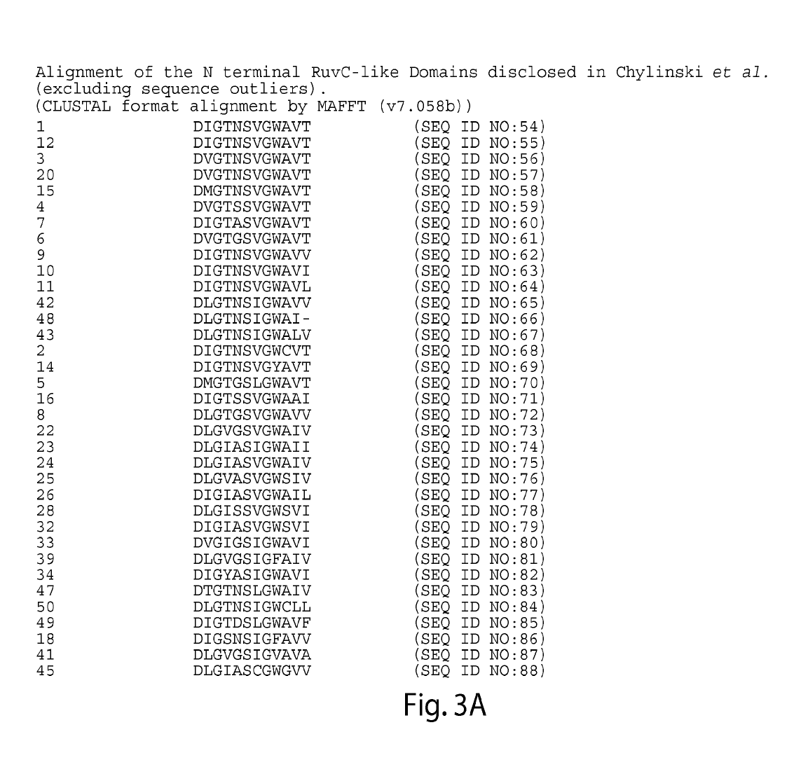

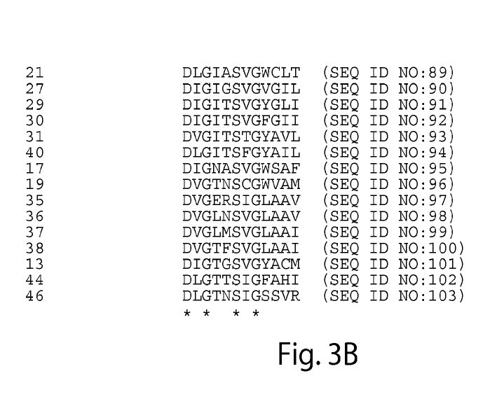

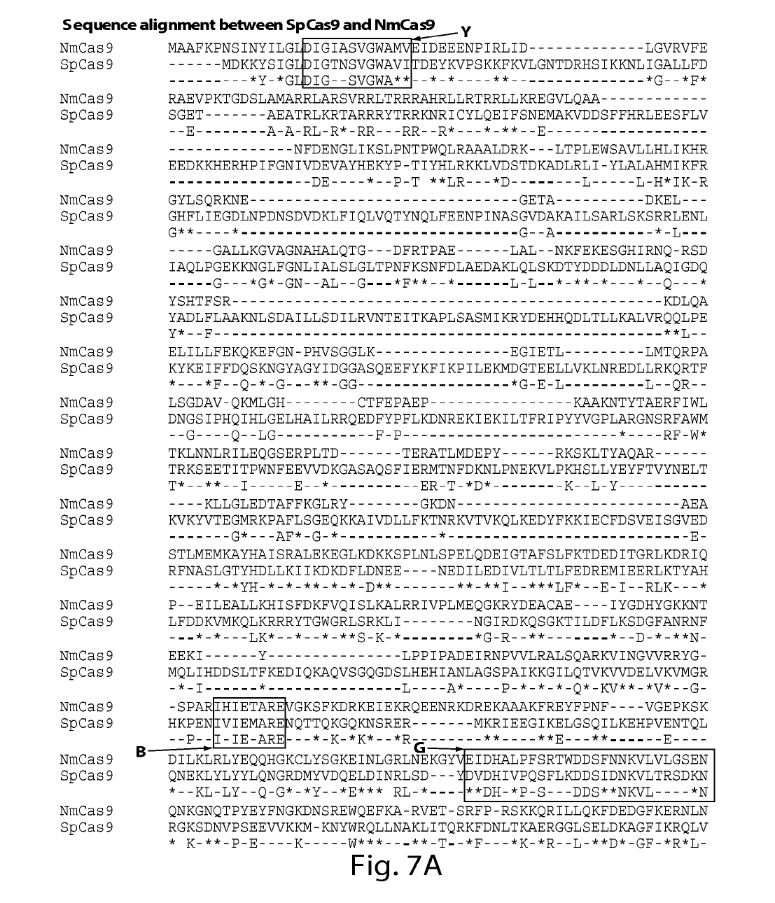

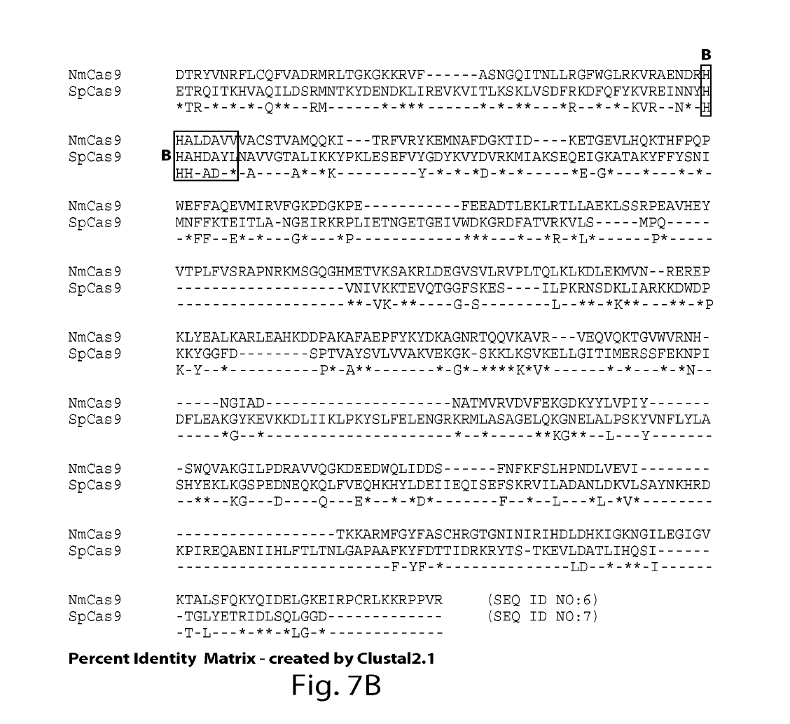

In an embodiment, the N-terminal RuvC-like domain differs from a sequence of an N-terminal RuvC like domain disclosed herein, e.g., in FIGS. 3A-3B or FIGS. 7A-7B, as many as 1 but no more than 2, 3, 4, or 5 residues. In an embodiment, 1, 2, 3 or all of the highly conserved residues identified in FIGS. 3A-3B or FIGS. 7A-7B are present.

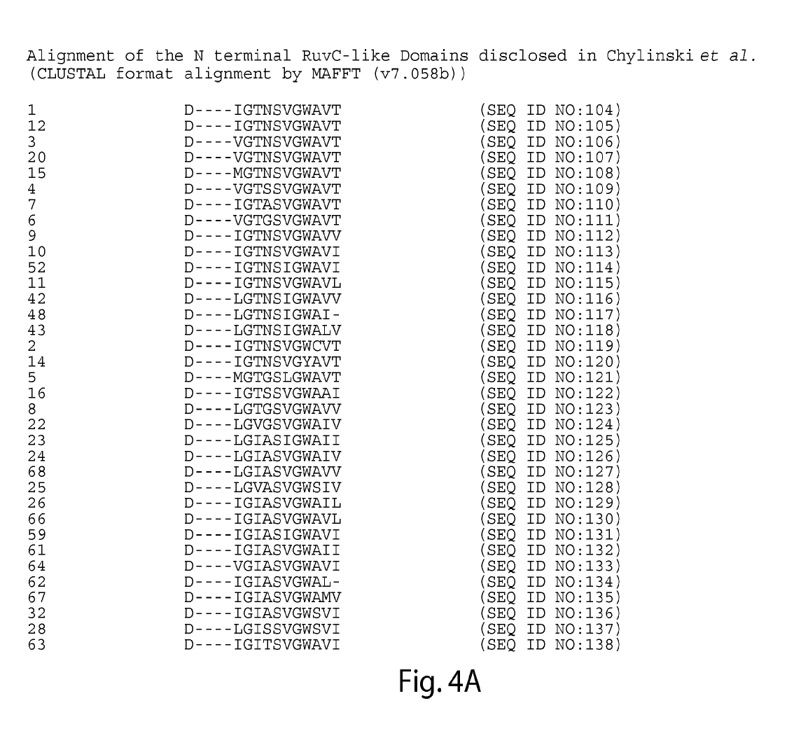

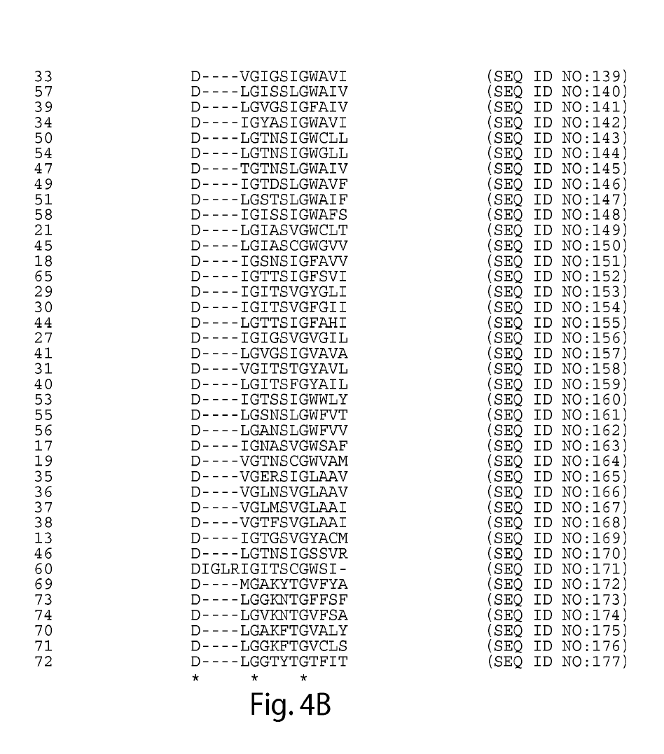

In an embodiment, the N-terminal RuvC-like domain differs from a sequence of an N-terminal RuvC-like domain disclosed herein, e.g., in FIGS. 4A-4B or FIGS. 7A-7B, as many as 1 but no more than 2, 3, 4, or 5 residues. In an embodiment, 1, 2, or all of the highly conserved residues identified in FIGS. 4A-4B or FIGS. 7A-7B are present.

Additional RuvC-Like Domains

In addition to the N-terminal RuvC-like domain, the Cas9 molecule or Cas9 polypeptide, e.g., an eaCas9 molecule or eaCas9 polypeptide, can comprise one or more additional RuvC-like domains. In an embodiment, the Cas9 molecule or Cas9 polypeptide can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

An additional RuvC-like domain can comprise an amino acid sequence:

I-X1-X2-E-X3-A-R-E (SEQ ID NO:12), wherein

X1 is V or H,

X2 is I, L or V (e.g., I or V); and

X3 is M or T.

In an embodiment, the additional RuvC-like domain comprises the amino acid sequence:

I—V-X2-E-M-A-R-E (SEQ ID NO:13), wherein

X2 is I, L or V (e.g., I or V) (e.g., the eaCas9 molecule or eaCas9 polypeptide can comprise an additional RuvC-like domain shown in FIG. 2A-2G or FIGS. 7A-7B (depicted as B)).

An additional RuvC-like domain can comprise an amino acid sequence:

H-H-A-X1-D-A-X2-X3 (SEQ ID NO: 14), wherein

X1 is H or L;

X2 is R or V; and

X3 is E or V.

In an embodiment, the additional RuvC-like domain comprises the amino acid sequence:

(SEQ ID NO: 15)

H-H-A-H-D-A-Y-L.

In an embodiment, the additional RuvC-like domain differs from a sequence of SEQ ID NO: 12, 13, 14 or 15 by as many as 1 but no more than 2, 3, 4, or 5 residues.

In some embodiments, the sequence flanking the N-terminal RuvC-like domain is a sequence of formula V:

(SEQ ID NO: 16)

K-X1′-Y-X2′-X3′-X4′-Z-T-D-X9′-Y,.

wherein

X1′ is selected from K and P,

X2′ is selected from V, L, I, and F (e.g., V, I and L);

X3′ is selected from G, A and S (e.g., G),

X4′ is selected from L, I, V and F (e.g., L);

X9′ is selected from D, E, N and Q; and

Z is an N-terminal RuvC-like domain, e.g., as described above.

HNH-Like Domains

In an embodiment, an HNH-like domain cleaves a single stranded complementary domain, e.g., a complementary strand of a double stranded nucleic acid molecule. In an embodiment, an HNH-like domain is at least 15, 20, 25 amino acids in length but not more than 40, 35 or 30 amino acids in length, e.g., 20 to 35 amino acids in length, e.g., 25 to 30 amino acids in length. Exemplary HNH-like domains are described below.

In an embodiment, an eaCas9 molecule or eaCas9 polypeptide comprises an HNH-like domain having an amino acid sequence of formula VI:

X1-X2-X3-H-X4-X5-P-X6-X7-X8-X9-X10-X11-X12-X13-X14-X15-N-X16-X17-X18-X19-X20-X21-X22-X23-N(SEQ ID NO: 17), wherein

X1 is selected from D, E, Q and N (e.g., D and E);

X2 is selected from L, I, R, Q, V, M and K;

X3 is selected from D and E;

X4 is selected from I, V, T, A and L (e.g., A, I and V);

X5 is selected from V, Y, I, L, F and W (e.g., V, I and L);

X6 is selected from Q, H, R, K, Y, I, L, F and W;

X7 is selected from S, A, D, T and K (e.g., S and A);

X8 is selected from F, L, V, K, Y, M, I, R, A, E, D and Q (e.g., F);

X9 is selected from L, R, T, I, V, S, C, Y, K, F and G;

X10 is selected from K, Q, Y, T, F, L, W, M, A, E, G, and S;

X11 is selected from D, S, N, R, L and T (e.g., D);

X12 is selected from D, N and S;

X13 is selected from S, A, T, G and R (e.g., S);

X14 is selected from I, L, F, S, R, Y, Q, W, D, K and H (e.g., I, L and F);

X15 is selected from D, S, I, N, E, A, H, F, L, Q, M, G, Y and V;

X16 is selected from K, L, R, M, T and F (e.g., L, R and K);

X17 is selected from V, L, I, A and T;

X18 is selected from L, I, V and A (e.g., L and I);

X19 is selected from T, V, C, E, S and A (e.g., T and V);

X20 is selected from R, F, T, W, E, L, N, C, K, V, S, Q, I, Y, H and A;

X21 is selected from S, P, R, K, N, A, H, Q, G and L;

X22 is selected from D, G, T, N, S, K, A, I, E, L, Q, R and Y; and

X23 is selected from K, V, A, E, Y, I, C, L, S, T, G, K, M, D and F.

In an embodiment, a HNH-like domain differs from a sequence of SEQ ID NO: 17 by at least one but no more than, 2, 3, 4, or 5 residues.

In an embodiment, the HNH-like domain is cleavage competent.

In an embodiment, the HNH-like domain is cleavage incompetent.

In an embodiment, an eaCas9 molecule or eaCas9 polypeptide comprises an HNH-like domain comprising an amino acid sequence of formula VII:

(SEQ ID NO: 18)

X1-X2-X3-H-X4-X5-P-X6-S-X8-X9-X10-D-D-S-X14-X15-N-
K-V-L-X19-X20-X21-X22-X23-N,

wherein

X1 is selected from D and E;

X2 is selected from L, I, R, Q, V, M and K;

X3 is selected from D and E;

X4 is selected from I, V, T, A and L (e.g., A, I and V);

X5 is selected from V, Y, I, L, F and W (e.g., V, I and L);

X6 is selected from Q, H, R, K, Y, I, L, F and W;

X8 is selected from F, L, V, K, Y, M, I, R, A, E, D and Q (e.g., F);

X9 is selected from L, R, T, I, V, S, C, Y, K, F and G;

X10 is selected from K, Q, Y, T, F, L, W, M, A, E, G, and S;

X14 is selected from I, L, F, S, R, Y, Q, W, D, K and H (e.g., I, L and F);

X15 is selected from D, S, I, N, E, A, H, F, L, Q, M, G, Y and V;

X19 is selected from T, V, C, E, S and A (e.g., T and V);

X20 is selected from R, F, T, W, E, L, N, C, K, V, S, Q, I, Y, H and A;

X21 is selected from S, P, R, K, N, A, H, Q, G and L;

X22 is selected from D, G, T, N, S, K, A, I, E, L, Q, R and Y; and

X23 is selected from K, V, A, E, Y, I, C, L, S, T, G, K, M, D and F.

In an embodiment, the HNH-like domain differs from a sequence of SEQ ID NO: 18 by 1, 2, 3, 4, or 5 residues.

In an embodiment, an eaCas9 molecule or eaCas9 polypeptide comprises an HNH-like domain comprising an amino acid sequence of formula VII:

(SEQ ID NO: 19)

X1-V-X3-H-I-V-P-X6-S-X8-X9-X10-D-D-S-X14-X15-N-K-
V-L-T-X20-X21-X22-X23-N,

wherein

X1 is selected from D and E;

X3 is selected from D and E;

X6 is selected from Q, H, R, K, Y, I, L and W;

X8 is selected from F, L, V, K, Y, M, I, R, A, E, D and Q (e.g., F);

X9 is selected from L, R, T, I, V, S, C, Y, K, F and G;

X10 is selected from K, Q, Y, T, F, L, W, M, A, E, G, and S;

X14 is selected from I, L, F, S, R, Y, Q, W, D, K and H (e.g., I, L and F);

X15 is selected from D, S, I, N, E, A, H, F, L, Q, M, G, Y and V;

X20 is selected from R, F, T, W, E, L, N, C, K, V, S, Q, I, Y, H and A;

X21 is selected from S, P, R, K, N, A, H, Q, G and L;

X22 is selected from D, G, T, N, S, K, A, I, E, L, Q, R and Y; and

X23 is selected from K, V, A, E, Y, I, C, L, S, T, G, K, M, D and F.

In an embodiment, the HNH-like domain differs from a sequence of SEQ ID NO: 19 by 1, 2, 3, 4, or 5 residues.

In an embodiment, an eaCas9 molecule or eaCas9 polypeptide comprises an HNH-like domain having an amino acid sequence of formula VIII:

(SEQ ID NO: 20)

D-X2-D-H-I-X5-P-Q-X7-F-X9-X10-D-X12-S-I-D-N-X16-V-
L-X19-X20-S-X22-X23-N,

wherein

X2 is selected from I and V;

X5 is selected from I and V;

X7 is selected from A and S;

X9 is selected from I and L;

X10 is selected from K and T;

X12 is selected from D and N;

X16 is selected from R, K and L; X19 is selected from T and V;

X20 is selected from S and R;

X22 is selected from K, D and A; and

X23 is selected from E, K, G and N (e.g., the eaCas9 molecule or eaCas9 polypeptide can comprise an HNH-like domain as described herein).

In an embodiment, the HNH-like domain differs from a sequence of SEQ ID NO: 20 by as many as 1 but no more than 2, 3, 4, or 5 residues.

In an embodiment, an eaCas9 molecule or eaCas9 polypeptide comprises the amino acid sequence of formula IX:

(SEQ ID NO: 21)

L-Y-Y-L-Q-N-G-X1′-D-M-Y-X2′-X3′-X4′-X5′-L-D-I-X6′-
X7′-L-S-X8′-Y-Z-N-R-X9′-K-X10′-D-X11′-V-P,

wherein

X1′ is selected from K and R;

X2′ is selected from V and T;

X3′ is selected from G and D;

X4′ is selected from E, Q and D;

X5′ is selected from E and D;

X6′ is selected from D, N and H;

X7′ is selected from Y, R and N;

X8′ is selected from Q, D and N; X9′ is selected from G and E;

X10′ is selected from S and G;

X11′ is selected from D and N; and

Z is an HNH-like domain, e.g., as described above.

In an embodiment, the eaCas9 molecule or eaCas9 polypeptide comprises an amino acid sequence that differs from a sequence of SEQ ID NO:21 by as many as 1 but no more than 2, 3, 4, or 5 residues.

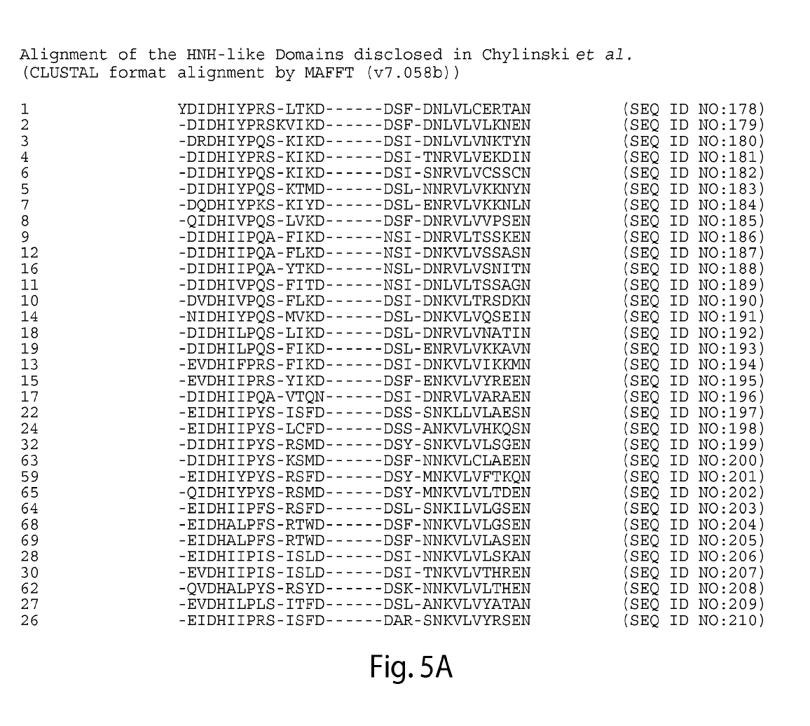

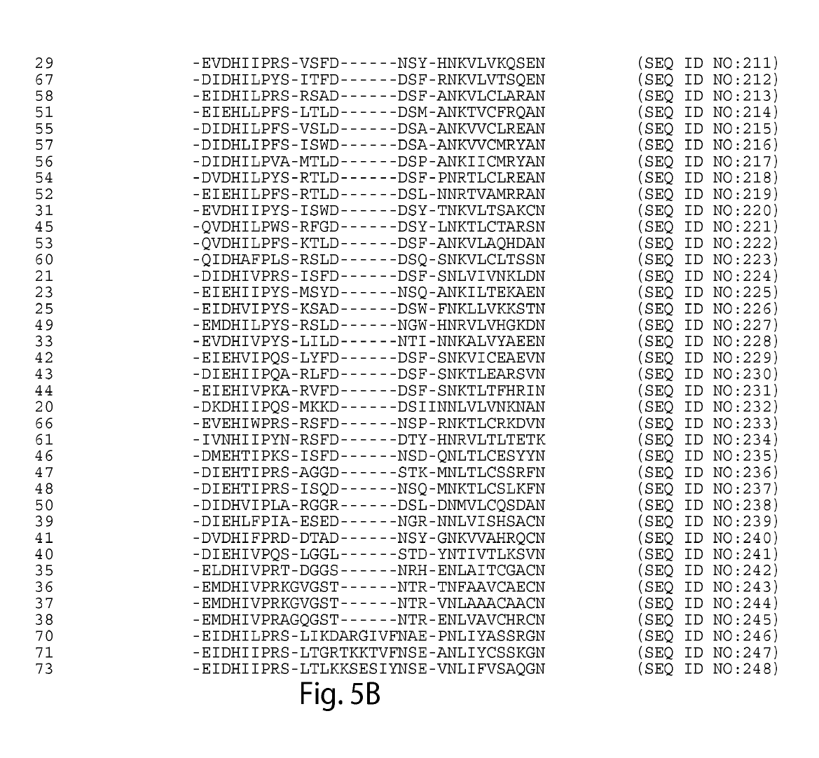

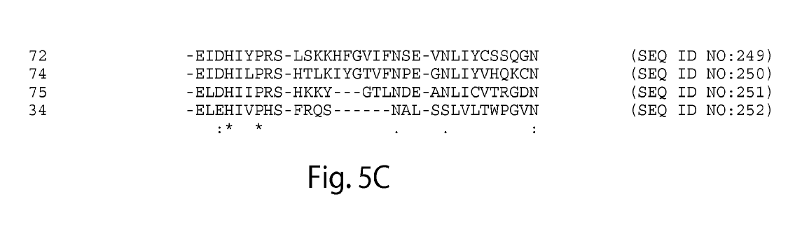

In an embodiment, the HNH-like domain differs from a sequence of an HNH-like domain disclosed herein, e.g., in FIGS. 5A-5C or FIGS. 7A-7B, as many as 1 but no more than 2, 3, 4, or 5 residues. In an embodiment, 1 or both of the highly conserved residues identified in FIGS. 5A-5C or FIGS. 7A-7B are present.

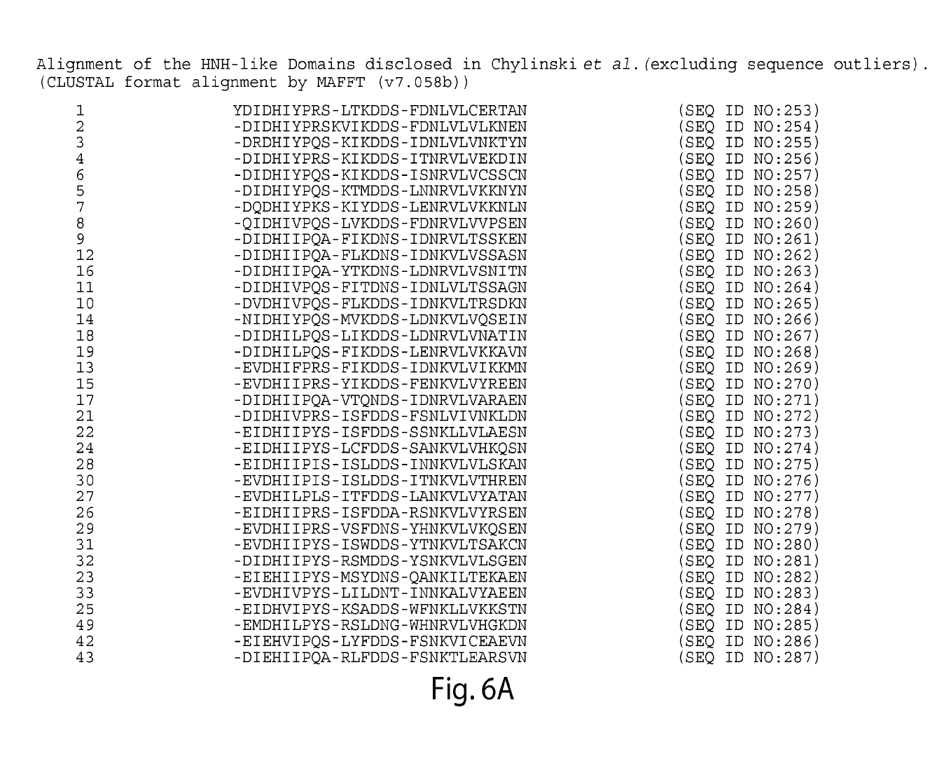

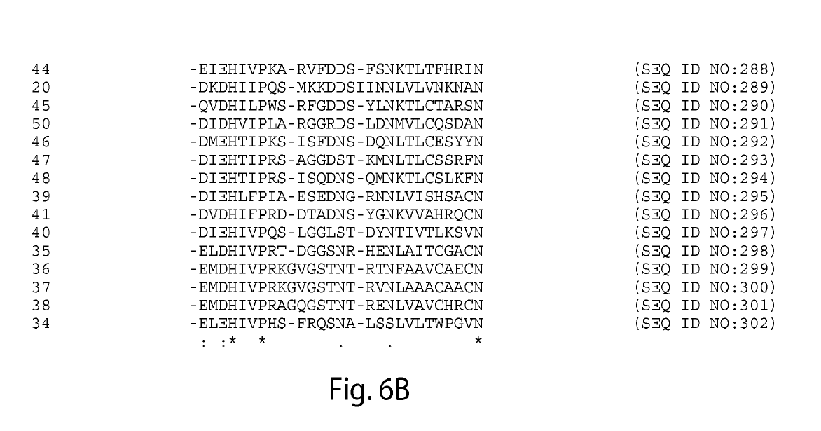

In an embodiment, the HNH-like domain differs from a sequence of an HNH-like domain disclosed herein, e.g., in FIGS. 6A-6B or FIGS. 7A-7B, as many as 1 but no more than 2, 3, 4, or 5 residues. In an embodiment, 1, 2, all 3 of the highly conserved residues identified in FIGS. 6A-6B or FIGS. 7A-7B are present.

Cas9 Activities

Nuclease and Helicase Activities

In an embodiment, the Cas9 molecule or Cas9 polypeptide is capable of cleaving a target nucleic acid molecule. Typically wild type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 polypeptide which is a nickase, or which lacks the ability to cleave target nucleic acid. A Cas9 molecule or Cas9 polypeptide that is capable of cleaving a target nucleic acid molecule is referred to herein as an eaCas9 (an enzymatically active Cas9) molecule or eaCas9 polypeptide. In an embodiment, an eaCas9 molecule or Cas9 polypeptide comprises one or more of the following activities:

a nickase activity, i.e., the ability to cleave a single strand, e.g., the non-complementary strand or the complementary strand, of a nucleic acid molecule;

a double stranded nuclease activity, i.e., the ability to cleave both strands of a double stranded nucleic acid and create a double stranded break, which in an embodiment is the presence of two nickase activities;

an endonuclease activity;

an exonuclease activity; and

a helicase activity, i.e., the ability to unwind the helical structure of a double stranded nucleic acid.

In an embodiment, an enzymatically active Cas9 or an eaCas9 molecule or an eaCas9 polypeptide cleaves both DNA strands and results in a double stranded break. In an embodiment, an eaCas9 molecule cleaves only one strand, e.g., the strand to which the gRNA hybridizes to, or the strand complementary to the strand the gRNA hybridizes with. In an embodiment, an eaCas9 molecule or eaCas9 polypeptide comprises cleavage activity associated with an HNH-like domain. In an embodiment, an eaCas9 molecule or eaCas9 polypeptide comprises cleavage activity associated with an N-terminal RuvC-like domain. In an embodiment, an eaCas9 molecule or eaCas9 polypeptide comprises cleavage activity associated with an HNH-like domain and cleavage activity associated with an N-terminal RuvC-like domain. In an embodiment, an eaCas9 molecule or eaCas9 polypeptide comprises an active, or cleavage competent, HNH-like domain and an inactive, or cleavage incompetent, N-terminal RuvC-like domain. In an embodiment, an eaCas9 molecule or eaCas9 polypeptide comprises an inactive, or cleavage incompetent, HNH-like domain and an active, or cleavage competent, N-terminal RuvC-like domain.

Some Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an eiCas9 molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

Targeting and PAMs

A Cas9 molecule or Cas9 polypeptide, is a polypeptide that can interact with a guide RNA (gRNA) molecule and, in concert with the gRNA molecule, localizes to a site which comprises a target domain and PAM sequence.

In an embodiment, the ability of an eaCas9 molecule or eaCas9 polypeptide to interact with and cleave a target nucleic acid is PAM sequence dependent. A PAM sequence is a sequence in the target nucleic acid. In an embodiment, cleavage of the target nucleic acid occurs upstream from the PAM sequence. EaCas9 molecules from different bacterial species can recognize different sequence motifs (e.g., PAM sequences). In an embodiment, an eaCas9 molecule of S. pyogenes recognizes the sequence motif NGG and directs cleavage of a target nucleic acid sequence 1 to 10, e.g., 3 to 5, base pairs upstream from that sequence. See, e.g., Mali et al., SCIENCE 2013; 339(6121): 823-826. In an embodiment, an eaCas9 molecule of S. thermophilus recognizes the sequence motif NGGNG and NNAGAAW (W=A or T) and directs cleavage of a core target nucleic acid sequence 1 to 10, e.g., 3 to 5, base pairs upstream from these sequences. See, e.g., Horvath et al., SCIENCE 2010; 327(5962):167-170, and Deveau et al., J BACTERIOL 2008; 190(4): 1390-1400. In an embodiment, an eaCas9 molecule of S. mutans recognizes the sequence motif NGG and/or NAAR (R=A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400. In an embodiment, an eaCas9 molecule of S. aureus recognizes the sequence motif NNGRR (R=A or G) and directs cleavage of a target nucleic acid sequence 1 to 10, e.g., 3 to 5, base pairs upstream from that sequence. In an embodiment, an eaCas9 molecule of S. aureus recognizes the sequence motif NNGRRN (R=A or G) and directs cleavage of a target nucleic acid sequence 1 to 10, e.g., 3 to 5, base pairs upstream from that sequence. In an embodiment, an eaCas9 molecule of S. aureus recognizes the sequence motif NNGRRT (R=A or G) and directs cleavage of a target nucleic acid sequence 1 to 10, e.g., 3 to 5, base pairs upstream from that sequence. In an embodiment, an eaCas9 molecule of S. aureus recognizes the sequence motif NNGRRV (R=A or G, V=A, G or C) and directs cleavage of a target nucleic acid sequence 1 to 10, e.g., 3 to 5, base pairs upstream from that sequence. In an embodiment, an eaCas9 molecule of Neisseria meningitidis recognizes the sequence motif NNNNGATT or NNNGCTT and directs cleavage of a target nucleic acid sequence 1 to 10, e.g., 3 to 5, base pairs upstream from that sequence. See, e.g., Hou et al., PNAS Early Edition 2013, 1-6. The ability of a Cas9 molecule to recognize a PAM sequence can be determined, e.g., using a transformation assay described in Jinek et al., SCIENCE 2012 337:816. In the aforementioned embodiments, N can be any nucleotide residue, e.g., any of A, G, C or T.

As is discussed herein, Cas9 molecules can be engineered to alter the PAM specificity of the Cas9 molecule.

Exemplary naturally occurring Cas9 molecules are described in Chylinski et al., RNA BIOLOGY 2013 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408). Additional exemplary Cas9 molecules are a Cas9 molecule of Neisseria meningitides (Hou et al., PNAS Early Edition 2013, 1-6 and a S. aureus cas9 molecule.

In an embodiment, a Cas9 molecule or Cas9 polypeptide, e.g., an eaCas9 molecule or eaCas9 polypeptide, comprises an amino acid sequence:

having 60%, 65%, 70%, 75%, 80%, 85%, 90%, 95%, 96%, 97%, 98% or 99% homology with;

differs at no more than, 2, 5, 10, 15, 20, 30, or 40% of the amino acid residues when compared with;

differs by at least 1, 2, 5, 10 or 20 amino acids, but by no more than 100, 80, 70, 60, 50, 40 or 30 amino acids from; or

is identical to any Cas9 molecule sequence described herein, or a naturally occurring Cas9 molecule sequence, e.g., a Cas9 molecule from a species listed herein or described in Chylinski et al., RNA BIOLOGY 2013 10:5, 727-737; Hou et al., PNAS Early Edition 2013, 1-6; SEQ ID NO:1-4. In an embodiment, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to localize to a target nucleic acid.

In an embodiment, a Cas9 molecule or Cas9 polypeptide comprises any of the amino acid sequence of the consensus sequence of FIGS. 2A-2G, wherein “*” indicates any amino acid found in the corresponding position in the amino acid sequence of a Cas9 molecule of S. pyogenes, S. thermophilus, S. mutans and L. innocua, and “-” indicates any amino acid. In an embodiment, a Cas9 molecule or Cas9 polypeptide differs from the sequence of the consensus sequence disclosed in FIGS. 2A-2G by at least 1, but no more than 2, 3, 4, 5, 6, 7, 8, 9, or 10 amino acid residues. In an embodiment, a Cas9 molecule or Cas9 polypeptide comprises the amino acid sequence of SEQ ID NO:7 of FIGS. 7A-7B, wherein “*” indicates any amino acid found in the corresponding position in the amino acid sequence of a Cas9 molecule of S. pyogenes, or N. meningitides, “-” indicates any amino acid, and “-” indicates any amino acid or absent. In an embodiment, a Cas9 molecule or Cas9 polypeptide differs from the sequence of SEQ ID NO:6 or 7 disclosed in FIGS. 7A-7B by at least 1, but no more than 2, 3, 4, 5, 6, 7, 8, 9, or 10 amino acid residues.

A comparison of the sequence of a number of Cas9 molecules indicate that certain regions are conserved. These are identified below as:

region 1 (residues 1 to 180, or in the case of region 1′ residues 120 to 180)

region 2 (residues 360 to 480);

region 3 (residues 660 to 720);

region 4 (residues 817 to 900); and

region 5 (residues 900 to 960);

In an embodiment, a Cas9 molecule or Cas9 polypeptide comprises regions 1-5, together with sufficient additional Cas9 molecule sequence to provide a biologically active molecule, e.g., a Cas9 molecule having at least one activity described herein. In an embodiment, each of regions 1-5, independently, have 50%, 60%, 70%, 80%, 85%, 90%, 95%, 96%, 97%, 98% or 99% homology with the corresponding residues of a Cas9 molecule or Cas9 polypeptide described herein, e.g., a sequence from FIGS. 2A-2G or from FIGS. 7A-7B.

In an embodiment, a Cas9 molecule or Cas9 polypeptide, e.g., an eaCas9 molecule or eaCas9 polypeptide, comprises an amino acid sequence referred to as region 1:

having 50%, 60%, 70%, 80%, 85%, 90%, 95%, 96%, 97%, 98% or 99% homology with amino acids 1-180 (the numbering is according to the motif sequence in FIG. 2; 52% of residues in the four Cas9 sequences in FIGS. 2A-2G are conserved) of the amino acid sequence of Cas9 of S. pyogenes;

differs by at least 1, 2, 5, 10 or 20 amino acids but by no more than 90, 80, 70, 60, 50, 40 or 30 amino acids from amino acids 1-180 of the amino acid sequence of Cas9 of S. pyogenes, S. thermophilus, S. mutans or Listeria innocua; or

is identical to 1-180 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 1′:

having 55%, 60%, 65%, 70%, 75%, 80%, 85%, 90%, 95%, 96%, 97%, 98% or 99% homology with amino acids 120-180 (55% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 120-180 of the amino acid sequence of Cas9 of S. pyogenes, S. thermophilus, S. mutans or L. innocua; or

is identical to 120-180 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 2:

having 50%, 55%, 60%, 65%, 70%, 75%, 80%, 85%, 90%, 95%, 96%, 97%, 98% or 99% homology with amino acids 360-480 (52% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 360-480 of the amino acid sequence of Cas9 of S. pyogenes, S. thermophilus, S. mutans or L. innocua; or

is identical to 360-480 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 3:

having 55%, 60%, 65%, 70%, 75%, 80%, 85%, 90%, 95%, 96%, 97%, 98%, or 99% homology with amino acids 660-720 (56% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 660-720 of the amino acid sequence of Cas9 of S. pyogenes, S. thermophilus, S. mutans or L. innocua; or

is identical to 660-720 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 4:

having 50%, 55%, 60%, 65%, 70%, 75%, 80%, 85%, 90%, 95%, 96%, 97%, 98%, or 99% homology with amino acids 817-900 (55% of residues in the four Cas9 sequences in FIGS. 2A-2G are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 817-900 of the amino acid sequence of Cas9 of S. pyogenes, S. thermophilus, S. mutans or L. innocua; or

is identical to 817-900 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 5:

having 50%, 55%, 60%, 65%, 70%, 75%, 80%, 85%, 90%, 95%, 96%, 97%, 98%, or 99% homology with amino acids 900-960 (60% of residues in the four Cas9 sequences in FIGS. 2A-2G are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 900-960 of the amino acid sequence of Cas9 of S. pyogenes, S. thermophilus, S. mutans or L. innocua; or

is identical to 900-960 of the amino acid sequence of Cas9 of S. pyogenes, S. thermophilus, S. mutans or L. innocua.

Engineered or Altered Cas9 Molecules and Cas9 Polypeptides

Cas9 molecules and Cas9 polypeptides described herein, e.g., naturally occurring Cas9 molecules, can possess any of a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In an embodiment, a Cas9 molecule or Cas9 polypeptide can include all or a subset of these properties. In typical embodiments, a Cas9 molecule or Cas9 polypeptide have the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (engineered, as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In an embodiment an engineered Cas9 molecule or Cas9 polypeptide can have an alteration that alters its size, e.g., a deletion of amino acid sequence that reduces its size, e.g., without significant effect on one or more, or any Cas9 activity. In an embodiment, an engineered Cas9 molecule or Cas9 polypeptide can comprise an alteration that affects PAM recognition. E.g., an engineered Cas9 molecule can be altered to recognize a PAM sequence other than that recognized by the endogenous wild-type PI domain. In an embodiment, a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring Cas9 molecules or Cas9 polypeptides to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In an embodiment, a Cas9 molecule or Cas9 polypeptide can comprises one or more mutations or differences, e.g., at least 1, 2, 3, 4, 5, 10, 15, 20, 30, 40 or 50 mutations, but less than 200, 100, or 80 mutations relative to a reference, e.g., a parental, Cas9 molecule.

In an embodiment, a mutation or mutations do not have a substantial effect on a Cas9 activity, e.g. a Cas9 activity described herein. In an embodiment, a mutation or mutations have a substantial effect on a Cas9 activity, e.g. a Cas9 activity described herein.

Non-Cleaving and Modified-Cleavage Cas9 Molecules and Cas9 Polypeptides

In an embodiment, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

Modified Cleavage eaCas9 Molecules and eaCas9 Polypeptides

In an embodiment, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In an embodiment, an eaCas9 molecule or eaCas9 polypeptide comprises an active, or cleavage competent, HNH-like domain (e.g., an HNH-like domain described herein, e.g., SEQ ID NO: 17, SEQ ID NO: 18, SEQ ID NO: 19, SEQ ID NO: 20 or SEQ ID NO: 21) and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIGS. 2A-2G or an aspartic acid at position 10 of SEQ ID NO: 7, e.g., can be substituted with an alanine. In an embodiment, the eaCas9 molecule or eaCas9 polypeptide differs from wild type in the N-terminal RuvC-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

In an embodiment, an eaCas9 molecule or eaCas9 polypeptide comprises an inactive, or cleavage incompetent, HNH domain and an active, or cleavage competent, N-terminal RuvC-like domain (e.g., a RuvC-like domain described herein, e.g., SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16). Exemplary inactive, or cleavage incompetent HNH-like domains can have a mutation at one or more of: a histidine in an HNH-like domain, e.g., a histidine shown at position 856 of the consensus sequence disclosed in FIGS. 2A-2G, e.g., can be substituted with an alanine; and one or more asparagines in an HNH-like domain, e.g., an asparagine shown at position 870 of the consensus sequence disclosed in FIGS. 2A-2G and/or at position 879 of the consensus sequence disclosed in FIGS. 2A-2G, e.g., can be substituted with an alanine. In an embodiment, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

Alterations in the Ability to Cleave One or Both Strands of a Target Nucleic Acid

In an embodiment, exemplary Cas9 activities comprise one or more of PAM specificity, cleavage activity, and helicase activity. A mutation(s) can be present, e.g., in one or more RuvC-like domain, e.g., an N-terminal RuvC-like domain; an HNH-like domain; a region outside the RuvC-like domains and the HNH-like domain. In some embodiments, a mutation(s) is present in a RuvC-like domain, e.g., an N-terminal RuvC-like domain. In some embodiments, a mutation(s) is present in an HNH-like domain. In some embodiments, mutations are present in both a RuvC-like domain, e.g., an N-terminal RuvC-like domain and an HNH-like domain.

Exemplary mutations that may be made in the RuvC domain or HNH domain with reference to the S. pyogenes sequence include: D10A, E762A, H840A, N854A, N863A and/or D986A.

In an embodiment, a Cas9 molecule or Cas9 polypeptide is an eiCas9 molecule or eiCas9 polypeptide comprising one or more differences in a RuvC domain and/or in an HNH domain as compared to a reference Cas9 molecule, and the eiCas9 molecule or eiCas9 polypeptide does not cleave a nucleic acid, or cleaves with significantly less efficiency than does wildtype, e.g., when compared with wild type in a cleavage assay, e.g., as described herein, cuts with less than 50, 25, 10, or 1% of a reference Cas9 molecule, as measured by an assay described herein.

Whether or not a particular sequence, e.g., a substitution, may affect one or more activity, such as targeting activity, cleavage activity, etc., can be evaluated or predicted, e.g., by evaluating whether the mutation is conservative or by the method described in Section IV. In an embodiment, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In an embodiment, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S aureus, S. pyogenes, or C. jejuni as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S aureus, S. pyogenes, or C. jejuni); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S aureus, S. pyogenes, or C. jejuni); or the ability to cleave a nucleic acid molecule, e.g., a double stranded or single stranded nucleic acid molecule, can be eliminated.

In an embodiment, the altered Cas9 molecule or Cas9 polypeptide is an eaCas9 molecule or eaCas9 polypeptide comprising one or more of the following activities: cleavage activity associated with a RuvC domain; cleavage activity associated with an HNH domain; cleavage activity associated with an HNH domain and cleavage activity associated with a RuvC domain.

In an embodiment, the altered Cas9 molecule or Cas9 polypeptide is an eiCas9 molecule or eiCas9 polypeptide which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can be a naturally occurring unmodified Cas9 molecule, e.g., a naturally occurring Cas9 molecule such as a Cas9 molecule of S. pyogenes, S. thermophilus, S. aureus, C. jejuni or N. meningitidis. In an embodiment, the reference Cas9 molecule is the naturally occurring Cas9 molecule having the closest sequence identity or homology. In an embodiment, the eiCas9 molecule or eiCas9 polypeptide lacks substantial cleavage activity associated with a RuvC domain and cleavage activity associated with an HNH domain.

In an embodiment, the altered Cas9 molecule or Cas9 polypeptide is an eaCas9 molecule or eaCas9 polypeptide comprising the fixed amino acid residues of S. pyogenes shown in the consensus sequence disclosed in FIGS. 2A-2G, and has one or more amino acids that differ from the amino acid sequence of S. pyogenes (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G or SEQ ID NO: 7.

In an embodiment, the altered Cas9 molecule or Cas9 polypeptide comprises a sequence in which:

the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;
the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an S. pyogenes Cas9 molecule; and, the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an S. pyogenes Cas9 molecule.

In an embodiment, the altered Cas9 molecule or Cas9 polypeptide is an eaCas9 molecule or eaCas9 polypeptide comprising the fixed amino acid residues of S. thermophilus shown in the consensus sequence disclosed in FIGS. 2A-2G, and has one or more amino acids that differ from the amino acid sequence of S. thermophilus (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G.

In an embodiment, the altered Cas9 molecule or Cas9 polypeptide comprises a sequence in which:

the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;

the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an S. thermophilus Cas9 molecule; and,

the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an S. thermophilus Cas9 molecule.

In an embodiment, the altered Cas9 molecule or Cas9 polypeptide is an eaCas9 molecule or eaCas9 polypeptide comprising the fixed amino acid residues of S. mutans shown in the consensus sequence disclosed in FIGS. 2A-2G, and has one or more amino acids that differ from the amino acid sequence of S. mutans (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G.

In an embodiment, the altered Cas9 molecule or Cas9 polypeptide comprises a sequence in which:

the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;

the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an S. mutans Cas9 molecule; and,

the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an S. mutans Cas9 molecule.

In an embodiment, the altered Cas9 molecule or Cas9 polypeptide is an eaCas9 molecule or eaCas9 polypeptide comprising the fixed amino acid residues of L. innocula shown in the consensus sequence disclosed in FIGS. 2A-2G, and has one or more amino acids that differ from the amino acid sequence of L. innocula (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G.

In an embodiment, the altered Cas9 molecule or Cas9 polypeptide comprises a sequence in which:

the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;

the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an L. innocula Cas9 molecule; and,

the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an L. innocula Cas9 molecule.

In an embodiment, the altered Cas9 molecule or Cas9 polypeptide, e.g., an eaCas9 molecule or eaCas9 polypeptide, can be a fusion, e.g., of two of more different Cas9 molecules, e.g., of two or more naturally occurring Cas9 molecules of different species. For example, a fragment of a naturally occurring Cas9 molecule of one species can be fused to a fragment of a Cas9 molecule of a second species. As an example, a fragment of a Cas9 molecule of S. pyogenes comprising an N-terminal RuvC-like domain can be fused to a fragment of Cas9 molecule of a species other than S. pyogenes (e.g., S. thermophilus) comprising an HNH-like domain.

Cas9 Molecules and Cas9 Polypeptides with Altered PAM Recognition or No PAM Recognition

Naturally occurring Cas9 molecules can recognize specific PAM sequences, for example, the PAM recognition sequences described above for S. pyogenes, S. thermophiles, S. mutans, S. aureus and N. meningitides.

In an embodiment, a Cas9 molecule or Cas9 polypeptide has the same PAM specificities as a naturally occurring Cas9 molecule. In other embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule recognizes to decrease off target sites and/or improve specificity; or eliminate a PAM recognition requirement. In an embodiment, a Cas9 molecule or Cas9 polypeptide can be altered, e.g., to increase length of PAM recognition sequence and/or improve Cas9 specificity to high level of identity (e.g., 98%, 99% or 100% match between gRNA and a PAM sequence), e.g., to decrease off target sites and increase specificity. In an embodiment, the length of the PAM recognition sequence is at least 4, 5, 6, 7, 8, 9, 10 or 15 amino acids in length. In an embodiment, the Cas9 specificity requires at least 90%, 95%, 96%, 97%, 98%, 99% or more homology between the gRNA and the PAM sequence. Cas9 molecules or Cas9 polypeptides that recognize different PAM sequences and/or have reduced off-target activity can be generated using directed evolution. Exemplary methods and systems that can be used for directed evolution of Cas9 molecules are described, e.g., in Esvelt et al. NATURE 2011, 472(7344): 499-503. Candidate Cas9 molecules can be evaluated, e.g., by methods described in Section IV.

Alterations of the PI domain, which mediates PAM recognition, are discussed below.

Synthetic Cas9 Molecules and Cas9 Polypeptides with Altered PI Domains

Current genome-editing methods are limited in the diversity of target sequences that can be targeted by the PAM sequence that is recognized by the Cas9 molecule utilized. A synthetic Cas9 molecule (or Syn-Cas9 molecule), or synthetic Cas9 polypeptide (or Syn-Cas9 polypeptide), as that term is used herein, refers to a Cas9 molecule or Cas9 polypeptide that comprises a Cas9 core domain from one bacterial species and a functional altered PI domain, i.e., a PI domain other than that naturally associated with the Cas9 core domain, e.g., from a different bacterial species.

In an embodiment, the altered PI domain recognizes a PAM sequence that is different from the PAM sequence recognized by the naturally-occurring Cas9 from which the Cas9 core domain is derived. In an embodiment, the altered PI domain recognizes the same PAM sequence recognized by the naturally-occurring Cas9 from which the Cas9 core domain is derived, but with different affinity or specificity. A Syn-Cas9 molecule or Syn-Cas9 polypeptide can be, respectively, a Syn-eaCas9 molecule or Syn-eaCas9 polypeptide or a Syn-eiCas9 molecule Syn-eiCas9 polypeptide.

An exemplary Syn-Cas9 molecule or Syn-Cas9 polypeptide comprises:

a) a Cas9 core domain, e.g., a Cas9 core domain from Table 8 or 9, e.g., a S. aureus, S. pyogenes, or C. jejuni Cas9 core domain; and

b) an altered PI domain from a species X Cas9 sequence selected from Tables 11 and 12.

In an embodiment, the RKR motif (the PAM binding motif) of said altered PI domain comprises: differences at 1, 2, or 3 amino acid residues; a difference in amino acid sequence at the first, second, or third position; differences in amino acid sequence at the first and second positions, the first and third positions, or the second and third positions; as compared with the sequence of the RKR motif of the native or endogenous PI domain associated with the Cas9 core domain.

In an embodiment, the Cas9 core domain comprises the Cas9 core domain from a species X Cas9 from Table 8 and said altered PI domain comprises a PI domain from a species Y Cas9 from Table 8.

In an embodiment, the RKR motif of the species X Cas9 is other than the RKR motif of the species Y Cas9.

In an embodiment, the RKR motif of the altered PI domain is selected from XXY, XNG, and XNQ.

In an embodiment, the altered PI domain has at least 60, 70, 80, 90, 95, or 100% homology with the amino acid sequence of a naturally occurring PI domain of said species Y from Table 8.

In an embodiment, the altered PI domain differs by no more than 50, 40, 30, 25, 20, 15, 10, 5, 4, 3, 2, or 1 amino acid residue from the amino acid sequence of a naturally occurring PI domain of said second species from Table 8.

In an embodiment, the Cas9 core domain comprises a S. aureus core domain and altered PI domain comprises: an A. denitrificans PI domain; a C. jejuni PI domain; a H. mustelae PI domain; or an altered PI domain of species X PI domain, wherein species X is selected from Table 12.

In an embodiment, the Cas9 core domain comprises a S. pyogenes core domain and the altered PI domain comprises: an A. denitrificans PI domain; a C. jejuni PI domain; a H. mustelae PI domain; or an altered PI domain of species X PI domain, wherein species X is selected from Table 12.

In an embodiment, the Cas9 core domain comprises a C. jejuni core domain and the altered PI domain comprises: an A. denitrificans PI domain; a H. mustelae PI domain; or an altered PI domain of species X PI domain, wherein species X is selected from Table 12.

In an embodiment, the Cas9 molecule or Cas9 polypeptide further comprises a linker disposed between said Cas9 core domain and said altered PI domain.

In an embodiment, the linker comprises: a linker described elsewhere herein disposed between the Cas9 core domain and the heterologous PI domain. Suitable linkers are further described in Section V.

Exemplary altered PI domains for use in Syn-Cas9 molecules are described in Tables 11 and 12. The sequences for the 83 Cas9 orthologs referenced in Tables 11 and 12 are provided in Table 8. Table 10 provides the Cas9 orthologs with known PAM sequences and the corresponding RKR motif.

In an embodiment, a Syn-Cas9 molecule or Syn-Cas9 polypeptide may also be size-optimized, e.g., the Syn-Cas9 molecule or Syn-Cas9 polypeptide comprises one or more deletions, and optionally one or more linkers disposed between the amino acid residues flanking the deletions. In an embodiment, a Syn-Cas9 molecule or Syn-Cas9 polypeptide comprises a REC deletion.

Size-Optimized Cas9 Molecules and Cas9 Polypeptides

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent to one of ordinary skill in the art upon review of this document.

A Cas9 molecule, e.g., a S. aureus, S. pyogenes, or C. jejuni, Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome-editing. A Cas9 molecule or Cas9 polypeptide can comprise one or more deletions that do not substantially affect or decrease the activity of the resultant Cas9 molecules or Cas9 polypeptides described herein. Activities that are retained in the Cas9 molecules or Cas9 polypeptides comprising a deletion as described herein include one or more of the following:

a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in an embodiment is the presence of two nickase activities;

an endonuclease activity;

an exonuclease activity;

a helicase activity, i.e., the ability to unwind the helical structure of a double stranded nucleic acid;

and recognition activity of a nucleic acid molecule, e.g., a target nucleic acid or a gRNA.

Activity of the Cas9 molecules or Cas9 polypeptides described herein can be assessed using the activity assays described herein or in the art.

Identifying Regions Suitable for Deletion

Suitable regions of Cas9 molecules for deletion can be identified by a variety of methods. Naturally-occurring orthologous Cas9 molecules from various bacterial species, e.g., any one of those listed in Table 8, can be modeled onto the crystal structure of S. pyogenes Cas9 (Nishimasu et al., Cell, 156:935-949, 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

REC-Optimized Cas9 Molecules and Cas9 Polypeptides

A REC-optimized Cas9 molecule, or a REC-optimized Cas9 polypeptide, as that term is used herein, refers to a Cas9 molecule or Cas9 polypeptide that comprises a deletion in one or both of the REC2 domain and the RE1_{CT} domain (collectively a REC deletion), wherein the deletion comprises at least 10% of the amino acid residues in the cognate domain. A REC-optimized Cas9 molecule or Cas9 polypeptide can be an eaCas9 molecule or eaCas9 polypeptide, or an eiCas9 molecule or eiCas9 polypeptide. An exemplary REC-optimized Cas9 molecule or REC-optimized Cas9 polypeptide comprises:

a) a deletion selected from:
i) a REC2 deletion;
ii) a REC1_{CT} deletion; or
iii) a REC1_{SUB} deletion.

Optionally, a linker is disposed between the amino acid residues that flank the deletion. In an embodiment, a Cas9 molecule or Cas9 polypeptide includes only one deletion, or only two deletions. A Cas9 molecule or Cas9 polypeptide can comprise a REC2 deletion and a REC1_{CT} deletion. A Cas9 molecule or Cas9 polypeptide can comprise a REC2 deletion and a REC1_{SUB} deletion.

Generally, the deletion will contain at least 10% of the amino acids in the cognate domain, e.g., a REC2 deletion will include at least 10% of the amino acids in the REC2 domain.

A deletion can comprise: at least 10, 20, 30, 40, 50, 60, 70, 80, or 90% of the amino acid residues of its cognate domain; all of the amino acid residues of its cognate domain; an amino acid residue outside its cognate domain; a plurality of amino acid residues outside its cognate domain; the amino acid residue immediately N terminal to its cognate domain; the amino acid residue immediately C terminal to its cognate domain; the amino acid residue immediately N terminal to its cognate and the amino acid residue immediately C terminal to its cognate domain; a plurality of, e.g., up to 5, 10, 15, or 20, amino acid residues N terminal to its cognate domain; a plurality of, e.g., up to 5, 10, 15, or 20, amino acid residues C terminal to its cognate domain; a plurality of, e.g., up to 5, 10, 15, or 20, amino acid residues N terminal to its cognate domain and a plurality of e.g., up to 5, 10, 15, or 20, amino acid residues C terminal to its cognate domain.

In an embodiment, a deletion does not extend beyond: its cognate domain; the N terminal amino acid residue of its cognate domain; the C terminal amino acid residue of its cognate domain.

A REC-optimized Cas9 molecule or REC-optimized Cas9 polypeptide can include a linker disposed between the amino acid residues that flank the deletion. Any linkers known in the art that maintain the conformation or native fold of the Cas9 molecule (thereby retaining Cas9 activity) can be used between the amino acid resides that flank a REC deletion in a REC-optimized Cas9 molecule or REC-optimized Cas9 polypeptide. Linkers for use in generating recombinant proteins, e.g., multi-domain proteins, are known in the art (Chen et al., Adv Drug Delivery Rev, 65:1357-69, 2013).

In an embodiment, a REC-optimized Cas9 molecule or REC-optimized Cas9 polypeptide comprises an amino acid sequence that, other than any REC deletion and associated linker, has at least 50, 55, 60, 65, 70, 75, 80, 85, 90, 95, 99, or 100% homology with the amino acid sequence of a naturally occurring Cas9, e.g., a Cas9 molecule described in Table 8, e.g., a S. aureus Cas9 molecule, a S. pyogenes Cas9 molecule, or a C. jejuni Cas9 molecule.

In an embodiment, a REC-optimized Cas9 molecule or REC-optimized Cas9 polypeptide comprises an amino acid sequence that, other than any REC deletion and associated linker, differs by no more than 1, 2, 3, 4, 5, 6, 7, 8, 9, 10, 15, 20, or 25, amino acid residues from the amino acid sequence of a naturally occurring Cas 9, e.g., a Cas9 molecule described in Table 8, e.g., a S. aureus Cas9 molecule, a S. pyogenes Cas9 molecule, or a C. jejuni Cas9 molecule.

In an embodiment, a REC-optimized Cas9 molecule or REC-optimized Cas9 polypeptide comprises an amino acid sequence that, other than any REC deletion and associate linker, differs by no more than 1, 2, 3, 4, 5, 6, 7, 8, 9, 10, 15, 20, or 25% of the, amino acid residues from the amino acid sequence of a naturally occurring Cas 9, e.g., a Cas9 molecule described in Table 8, e.g., a S. aureus Cas9 molecule, a S. pyogenes Cas9 molecule, or a C. jejuni Cas9 molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Sequence information for exemplary REC deletions are provided for 83 naturally-occurring Cas9 orthologs in Table 8.

The amino acid sequences of exemplary Cas9 molecules from different bacterial species are shown below.

TABLE 8
Amino Acid Sequence of Cas9 Orthologs

| | | REC2 | | | REC1_{CT} | | | Rec_{sub} | | |
|---|---|---|---|---|---|---|---|---|---|---|
| | Amino | start | stop | # AA | start | stop | # AA | start | stop | # AA |
| | acid | (AA | (AA | deleted | (AA | (AA | deleted | (AA | (AA | deleted |
| Species/Composite ID | sequence | pos) | pos) | (n) | pos) | pos) | (n) | pos) | pos) | (n) |
| Staphylococcus Aureus | SEQ ID | 126 | 166 | 41 | 296 | 352 | 57 | 296 | 352 | 57 |
| tr\|J7RUA5\|J7RUA5_STAAU | NO: 304 | | | | | | | | | |
| Streptococcus Pyogenes | SEQ ID | 176 | 314 | 139 | 511 | 592 | 82 | 511 | 592 | 82 |
| sp\|Q99ZW2\|CAS9_STRP1 | NO: 305 | | | | | | | | | |
| Campylobacter jejuni NCTC 11168 | SEQ ID | 137 | 181 | 45 | 316 | 360 | 45 | 316 | 360 | 45 |
| gi\|218563121\|ref\|YP_002344900.1 | NO: 306 | | | | | | | | | |
| Bacteroides fragilis NCTC 9343 | SEQ ID | 148 | 339 | 192 | 524 | 617 | 84 | 524 | 617 | 84 |
| gi\|60683389\|ref\|YP_213533.1\| | NO: 307 | | | | | | | | | |
| Bifidobacterium bifidum S17 | SEQ ID | 173 | 335 | 163 | 516 | 607 | 87 | 516 | 607 | 87 |
| gi\|310286728\|ref\|YP_003937986. | NO: 308 | | | | | | | | | |
| Veillonella atypica ACS-134-V-Col7a | SEQ ID | 185 | 339 | 155 | 574 | 663 | 79 | 574 | 663 | 79 |
| gi\|303229466\|ref\|ZP_07316256.1 | NO: 309 | | | | | | | | | |
| Lactobacillus rhamnosus GG | SEQ ID | 169 | 320 | 152 | 559 | 645 | 78 | 559 | 645 | 78 |
| gi\|258509199\|ref\|YP_003171950.1 | NO: 310 | | | | | | | | | |
| Filifactor alocis ATCC 35896 | SEQ ID | 166 | 314 | 149 | 508 | 592 | 76 | 508 | 592 | 76 |
| gi\|374307738\|ref\|YP_005054169.1 | NO: 311 | | | | | | | | | |
| Oenococcus kitaharae DSM 17330 | SEQ ID | 169 | 317 | 149 | 555 | 639 | 80 | 555 | 639 | 80 |
| gi\|366983953\|gb\|EHN59352.1\| | NO: 312 | | | | | | | | | |
| Fructobacillus fructosus KCTC 3544 | SEQ ID | 168 | 314 | 147 | 488 | 571 | 76 | 488 | 571 | 76 |
| gi\|339625081\|ref\|ZP_08660870.1 | NO: 313 | | | | | | | | | |
| Catenibacterium mitsuokai DSM 15897 | SEQ ID | 173 | 318 | 146 | 511 | 594 | 78 | 511 | 594 | 78 |
| gi\|224543312\|ref\|ZP_03683851.1 | NO: 314 | | | | | | | | | |
| Finegoldia magna ATCC 29328 | SEQ ID | 168 | 313 | 146 | 452 | 534 | 77 | 452 | 534 | 77 |
| gi\|169823755\|ref\|YP_001691366.1 | NO: 315 | | | | | | | | | |
| CoriobacteriumglomeransPW2 | SEQ ID | 175 | 318 | 144 | 511 | 592 | 82 | 511 | 592 | 82 |
| gi\|328956315\|ref\|YP_004373648.1 | NO: 316 | | | | | | | | | |
| Eubacterium yurii ATCC 43715 | SEQ ID | 169 | 310 | 142 | 552 | 633 | 76 | 552 | 633 | 76 |
| gi\|306821691\|ref\|ZP_07455288.1 | NO: 317 | | | | | | | | | |
| Peptoniphilus duerdenii ATCC BAA-1640 | SEQ ID | 171 | 311 | 141 | 535 | 615 | 76 | 535 | 615 | 76 |
| gi\|304438954\|ref\|ZP_07398877.1 | NO: 318 | | | | | | | | | |
| Acidaminococcus sp. D21 | SEQ ID | 167 | 306 | 140 | 511 | 591 | 75 | 511 | 591 | 75 |
| gi\|227824983\|ref\|ZP_03989815.1 | NO: 319 | | | | | | | | | |
| Lactobacillus farciminis KCTC 3681 | SEQ ID | 171 | 310 | 140 | 542 | 621 | 85 | 542 | 621 | 85 |
| gi\|336394882\|ref\|ZP_08576281.1 | NO: 320 | | | | | | | | | |
| Streptococcus sanguinis SK49 | SEQ ID | 185 | 324 | 140 | 411 | 490 | 85 | 411 | 490 | 85 |
| gi\|422884106\|ref\|ZP_16930555.1 | NO: 321 | | | | | | | | | |
| Coprococcus catus GD-7 | SEQ ID | 172 | 310 | 139 | 556 | 634 | 76 | 556 | 634 | 76 |
| gi\|291520705\|emb\|CBK78998.1\| | NO: 322 | | | | | | | | | |
| Streptococcus mutans UA159 | SEQ ID | 176 | 314 | 139 | 392 | 470 | 84 | 392 | 470 | 84 |
| gi\|24379809\|ref\|NP_721764.1\| | NO: 323 | | | | | | | | | |
| Streptococcus pyogenes M1 GAS | SEQ ID | 176 | 314 | 139 | 523 | 600 | 82 | 523 | 600 | 82 |
| gi\|13622193\|gb\|AAK33936.1\| | NO: 324 | | | | | | | | | |
| Streptococcus thermophilus LMD-9 | SEQ ID | 176 | 314 | 139 | 481 | 558 | 81 | 481 | 558 | 81 |
| gi\|116628213\|ref\|YP_820832.1\| | NO: 325 | | | | | | | | | |
| Fusobacteriumnucleatum ATCC49256 | SEQ ID | 171 | 308 | 138 | 537 | 614 | 76 | 537 | 614 | 76 |
| gi\|34762592\|ref\|ZP_00143587.1\| | NO: 326 | | | | | | | | | |
| Planococcus antarcticus DSM 14505 | SEQ ID | 162 | 299 | 138 | 538 | 614 | 94 | 538 | 614 | 94 |
| gi\|389815359\|ref\|ZP_10206685.1 | NO: 327 | | | | | | | | | |
| Treponema denticola ATCC 35405 | SEQ ID | 169 | 305 | 137 | 524 | 600 | 81 | 524 | 600 | 81 |
| gi\|42525843\|ref\|NP_970941.1\| | NO: 328 | | | | | | | | | |
| Solobacterium moorei F0204 | SEQ ID | 179 | 314 | 136 | 544 | 619 | 77 | 544 | 619 | 77 |
| gi\|320528778\|ref\|ZP_08029929.1 | NO: 329 | | | | | | | | | |
| Staphylococcus pseudintermedius ED99 | SEQ ID | 164 | 299 | 136 | 531 | 606 | 92 | 531 | 606 | 92 |
| gi\|323463801\|gb\|ADX75954.1\| | NO: 330 | | | | | | | | | |
| Flavobacterium branchiophilum FL-15 | SEQ ID | 162 | 286 | 125 | 538 | 613 | 63 | 538 | 613 | 63 |
| gi\|347536497\|ref\|YP_004843922.1 | NO: 331 | | | | | | | | | |
| Ignavibacterium album JCM 16511 | SEQ ID | 223 | 329 | 107 | 357 | 432 | 90 | 357 | 432 | 90 |
| gi\|385811609\|ref\|YP_005848005.1 | NO: 332 | | | | | | | | | |
| Bergeyella zoohelcum ATCC 43767 | SEQ ID | 165 | 261 | 97 | 529 | 604 | 56 | 529 | 604 | 56 |
| gi\|423317190\|ref\|ZP_17295095.1 | NO: 333 | | | | | | | | | |
| Nitrobacter hamburgensis X14 | SEQ ID | 169 | 253 | 85 | 536 | 611 | 48 | 536 | 611 | 48 |
| gi\|92109262\|ref\|YP_571550.1\| | NO: 334 | | | | | | | | | |
| Odoribacter laneus YIT 12061 | SEQ ID | 164 | 242 | 79 | 535 | 610 | 63 | 535 | 610 | 63 |
| gi\|374384763\|ref\|ZP_09642280.1 | NO: 335 | | | | | | | | | |
| Legionella pneumophila str. Paris | SEQ ID | 164 | 239 | 76 | 402 | 476 | 67 | 402 | 476 | 67 |
| gi\|54296138\|ref\|YP_122507.1\| | NO: 336 | | | | | | | | | |
| Bacteroides sp. 20 3 | SEQ ID | 198 | 269 | 72 | 530 | 604 | 83 | 530 | 604 | 83 |
| gi\|301311869\|ref\|ZP_07217791.1 | NO: 337 | | | | | | | | | |
| Akkermansia muciniphila ATCC BAA-835 | SEQ ID | 136 | 202 | 67 | 348 | 418 | 62 | 348 | 418 | 62 |
| gi\|187736489\|ref\|YP_001878601 | NO: 338 | | | | | | | | | |
| Prevotella sp. C561 | SEQ ID | 184 | 250 | 67 | 357 | 425 | 78 | 357 | 425 | 78 |
| gi\|345885718\|ref\|ZP_08837074.1 | NO: 339 | | | | | | | | | |
| Wolinella succinogenes DSM 1740 | SEQ ID | 157 | 218 | 36 | 401 | 468 | 60 | 401 | 468 | 60 |
| gi\|34557932\|ref\|NP_907747.1\| | NO: 340 | | | | | | | | | |
| Alicyclobacillus hesperidum URH17-3-68 | SEQ ID | 142 | 196 | 55 | 416 | 482 | 61 | 416 | 482 | 61 |
| gi\|403744858\|ref\|ZP_10953934.1 | NO: 341 | | | | | | | | | |
| Caenispirillum salinarum AK4 | SEQ ID | 161 | 214 | 54 | 330 | 393 | 68 | 330 | 393 | 68 |
| gi\|427429481\|ref\|ZP_18919511.1 | NO: 342 | | | | | | | | | |
| Eubacterium rectale ATCC 33656 | SEQ ID | 133 | 185 | 53 | 322 | 384 | 60 | 322 | 384 | 60 |
| gi\|238924075\|ref\|YP_002937591.1 | NO: 343 | | | | | | | | | |
| Mycoplasma synoviae 53 | SEQ ID | 187 | 239 | 53 | 319 | 381 | 80 | 319 | 381 | 80 |
| gi\|71894592\|ref\|YP_278700.1\| | NO: 344 | | | | | | | | | |
| Porphyromonas sp. oral taxon 279 str. F0450 | SEQ ID | 150 | 202 | 53 | 309 | 371 | 60 | 309 | 371 | 60 |
| gi\|402847315\|ref\|ZP_10895610.1 | NO: 345 | | | | | | | | | |
| Streptococcus thermophilus LMD-9 | SEQ ID | 127 | 178 | 139 | 424 | 486 | 81 | 424 | 486 | 81 |
| gi\|116627542\|ref\|YP_820161.1\| | NO: 346 | | | | | | | | | |
| Roseburia inulinivorans DSM 16841 | SEQ ID | 154 | 204 | 51 | 318 | 380 | 69 | 318 | 380 | 69 |
| gi\|225377804\|ref\|ZP_03755025.1 | NO: 347 | | | | | | | | | |
| Methylosinus trichosporium OB3b | SEQ ID | 144 | 193 | 50 | 426 | 488 | 64 | 426 | 488 | 64 |
| gi\|296446027\|ref\|ZP_06887976.1 | NO: 348 | | | | | | | | | |
| Ruminococcus albus 8 | SEQ ID | 139 | 187 | 49 | 351 | 412 | 55 | 351 | 412 | 55 |
| gi\|325677756\|ref\|ZP_08157403.1 | NO: 349 | | | | | | | | | |
| Bifidobacterium longum DJO10A | SEQ ID | 183 | 230 | 48 | 370 | 431 | 44 | 370 | 431 | 44 |
| gi\|189440764\|ref\|YP_001955845 | NO: 350 | | | | | | | | | |
| Enterococcus faecalis TX0012 | SEQ ID | 123 | 170 | 48 | 327 | 387 | 60 | 327 | 387 | 60 |
| gi\|315149830\|gb\|EFT93846.1\| | NO: 351 | | | | | | | | | |
| Mycoplasma mobile 163K | SEQ ID | 179 | 226 | 48 | 314 | 374 | 79 | 314 | 374 | 79 |
| gi\|47458868\|ref\|YP_015730.1\| | NO: 352 | | | | | | | | | |
| Actinomyces coleocanis DSM 15436 | SEQ ID | 147 | 193 | 47 | 358 | 418 | 40 | 358 | 418 | 40 |
| gi\|227494853\|ref\|ZP_03925169.1 | NO: 353 | | | | | | | | | |
| Dinoroseobacter shibae DFL 12 | SEQ ID | 138 | 184 | 47 | 338 | 398 | 48 | 338 | 398 | 48 |
| gi\|159042956\|ref\|YP_001531750.1 | NO: 354 | | | | | | | | | |
| Actinomyces sp. oral taxon 180 str. F0310 | SEQ ID | 183 | 228 | 46 | 349 | 409 | 40 | 349 | 409 | 40 |
| gi\|315605738\|ref\|ZP_07880770.1 | NO: 355 | | | | | | | | | |
| Alcanivorax sp. W11-5 | SEQ ID | 139 | 183 | 45 | 344 | 404 | 61 | 344 | 404 | 61 |
| gi\|407803669\|ref\|ZP_11150502.1 | NO: 356 | | | | | | | | | |
| Aminomonas paucivorans DSM 12260 | SEQ ID | 134 | 178 | 45 | 341 | 401 | 63 | 341 | 401 | 63 |
| gi\|312879015\|ref\|ZP_07738815.1 | NO: 357 | | | | | | | | | |
| Mycoplasma canis PG 14 | SEQ ID | 139 | 183 | 45 | 319 | 379 | 76 | 319 | 379 | 76 |
| gi\|384393286\|gb\|EIE39736.1\| | NO: 358 | | | | | | | | | |
| Lactobacillus coryniformis KCTC 3535 | SEQ ID | 141 | 184 | 44 | 328 | 387 | 61 | 328 | 387 | 61 |
| gi\|336393381\|ref\|ZP_08574780.1 | NO: 359 | | | | | | | | | |
| Elusimicrobium minutum Pei191 | SEQ ID | 177 | 219 | 43 | 322 | 381 | 47 | 322 | 381 | 47 |
| gi\|187250660\|ref\|YP_001875142.1 | NO: 360 | | | | | | | | | |
| Neisseria meningitidis Z2491 | SEQ ID | 147 | 189 | 43 | 360 | 419 | 61 | 360 | 419 | 61 |
| gi\|218767588\|ref\|YP_002342100.1 | NO: 361 | | | | | | | | | |
| Pasteurella multocida str. Pm70 | SEQ ID | 139 | 181 | 43 | 319 | 378 | 61 | 319 | 378 | 61 |
| gi\|15602992\|ref\|NP_246064.1\| | NO: 362 | | | | | | | | | |
| Rhodovulum sp. PH10 | SEQ ID | 141 | 183 | 43 | 319 | 378 | 48 | 319 | 378 | 48 |
| gi\|402849997\|ref\|ZP_10898214.1 | NO: 363 | | | | | | | | | |
| Eubacterium dolichum DSM 3991 | SEQ ID | 131 | 172 | 42 | 303 | 361 | 59 | 303 | 361 | 59 |
| gi\|160915782\|ref\|ZP_02077990.1 | NO: 364 | | | | | | | | | |
| Nitratifractor salsuginis DSM 16511 | SEQ ID | 143 | 184 | 42 | 347 | 404 | 61 | 347 | 404 | 61 |
| gi\|319957206\|ref\|YP_004168469.1 | NO: 365 | | | | | | | | | |
| Rhodospirillum rubrum ATCC 11170 | SEQ ID | 139 | 180 | 42 | 314 | 371 | 55 | 314 | 371 | 55 |
| gi\|83591793\|ref\|YP_425545.1\| | NO: 366 | | | | | | | | | |
| Clostridium cellulolyticum H10 | SEQ ID | 137 | 176 | 40 | 320 | 376 | 61 | 320 | 376 | 61 |
| gi\|220930482\|ref\|YP_002507391.1 | NO: 367 | | | | | | | | | |
| Helicobacter mustelae 12198 | SEQ ID | 148 | 187 | 40 | 298 | 354 | 48 | 298 | 354 | 48 |
| gi\|291276265\|ref\|YP_003516037.1 | NO: 368 | | | | | | | | | |
| Ilyobacter polytropus DSM 2926 | SEQ ID | 134 | 173 | 40 | 462 | 517 | 63 | 462 | 517 | 63 |
| gi\|310780384\|ref\|YP_003968716.1 | NO: 369 | | | | | | | | | |
| Sphaerochaeta globus str. Buddy | SEQ ID | 163 | 202 | 40 | 335 | 389 | 45 | 335 | 389 | 45 |
| gi\|325972003\|ref\|YP_004248194.1 | NO: 370 | | | | | | | | | |
| Staphylococcus lugdunensis M23590 | SEQ ID | 128 | 167 | 40 | 337 | 391 | 57 | 337 | 391 | 57 |
| gi\|315659848\|ref\|ZP_07912707.1 | NO: 371 | | | | | | | | | |
| Treponema sp. JC4 | SEQ ID | 144 | 183 | 40 | 328 | 382 | 63 | 328 | 382 | 63 |
| gi\|384109266\|ref\|ZP_10010146.1 | NO: 372 | | | | | | | | | |
| uncultured delta proteobacterium | SEQ ID | 154 | 193 | 40 | 313 | 365 | 55 | 313 | 365 | 55 |
| HF0070 07E19 | NO: 373 | | | | | | | | | |
| gi\|297182908\|gb\|ADI19058.1\| | | | | | | | | | | |
| Alicycliphilus denitrificans K601 | SEQ ID | 140 | 178 | 39 | 317 | 366 | 48 | 317 | 366 | 48 |
| gi\|330822845\|ref\|YP_004386148.1 | NO: 374 | | | | | | | | | |
| Azospirillum sp. B510 | SEQ ID | 205 | 243 | 39 | 342 | 389 | 46 | 342 | 389 | 46 |
| gi\|288957741\|ref\|YP_003448082.1 | NO: 375 | | | | | | | | | |
| Bradyrhizobium sp. BTAi1 | SEQ ID | 143 | 181 | 39 | 323 | 370 | 48 | 323 | 370 | 48 |
| gi\|148255343\|ref\|YP_001239928.1 | NO: 376 | | | | | | | | | |
| Parvibaculum lavamentivorans DS-1 | SEQ ID | 138 | 176 | 39 | 327 | 374 | 58 | 327 | 374 | 58 |
| gi\|154250555\|ref\|YP_001411379.1 | NO: 377 | | | | | | | | | |
| Prevotella timonensis CRIS 5C-B1 | SEQ ID | 170 | 208 | 39 | 328 | 375 | 61 | 328 | 375 | 61 |
| gi\|282880052\|ref\|ZP_06288774.1 | NO: 378 | | | | | | | | | |
| Bacillus smithii 7 3 47FAA | SEQ ID | 134 | 171 | 38 | 401 | 448 | 63 | 401 | 448 | 63 |
| gi\|365156657\|ref\|ZP_09352959.1 | NO: 379 | | | | | | | | | |
| Cand. Puniceispirillum marinum IMCC1322 | SEQ ID | 135 | 172 | 38 | 344 | 391 | 53 | 344 | 391 | 53 |
| gi\|294086111\|ref\|YP_003552871.1 | NO: 380 | | | | | | | | | |
| Barnesiella intestinihominis YIT 11860 | SEQ ID | 140 | 176 | 37 | 371 | 417 | 60 | 371 | 417 | 60 |
| gi\|404487228\|ref\|ZP_11022414.1 | NO: 381 | | | | | | | | | |
| Ralstonia syzygii R24 | SEQ ID | 140 | 176 | 37 | 395 | 440 | 50 | 395 | 440 | 50 |
| gi\|344171927\|emb\|CCA84553.1\| | NO: 382 | | | | | | | | | |
| Wolinella succinogenes DSM 1740 | SEQ ID | 145 | 180 | 36 | 348 | 392 | 60 | 348 | 392 | 60 |
| gi\|34557790\|ref\|NP_907605.1\| | NO: 383 | | | | | | | | | |
| Mycoplasma gallisepticum str. F | SEQ ID | 144 | 177 | 34 | 373 | 416 | 71 | 373 | 416 | 71 |
| gi\|284931710\|gb\|ADC31648.1\| | NO: 384 | | | | | | | | | |
| Acidothermus cellulolyticus 11B | SEQ ID | 150 | 182 | 33 | 341 | 380 | 58 | 341 | 380 | 58 |
| gi\|117929158\|ref\|YP_873709.1\| | NO: 385 | | | | | | | | | |
| Mycoplasma ovipneumoniae SC01 | SEQ ID | 156 | 184 | 29 | 381 | 420 | 62 | 381 | 420 | 62 |
| gi\|363542550\|ref\|ZP_09312133.1 | NO: 386 | | | | | | | | | |

TABLE 9
Amino Acid Sequence of Cas9 Core Domains

| | Cas9 Start (AA pos) | Cas9 Stop (AA pos) |
|---|---|---|
| | Start and Stop numbers refer to the | |
| Strain Name | sequence in Table 7 | |
| Staphylococcus Aureus | 1 | 772 |
| Streptococcus Pyogenes | 1 | 1099 |
| Campulobacter Jejuni | 1 | 741 |

TABLE 10
Identified PAM sequences and
corresponding RKR motifs

| | | RKR |
|---|---|---|
| | PAM sequence | motif |
| Strain Name | (NA) | (AA) |
| Streptococcus pyogenes | NGG | RKR |
| Streptococcus mutans | NGG | RKR |
| Streptococcus | NGGNG | RYR |
| thermophilus A | | |
| Treponema denticola | NAAAAN | VAK |
| Streptococcus | NNAAAAW | IYK |
| thermophilus B | | |
| Campylobacter jejuni | NNNNACA | NLK |
| Pasteurella multocida | GNNNCNNA | KDG |
| Neisseria meningitidis | NNNNGATT or | IGK |
| Staphylococcus aureus | NNGRRV (R = A or G; | NDK |
| | V = A, G or C) | |
| | NNGRRT (R = A or G) | |

PI domains are provided in Tables 11 and 12.

TABLE 11
Altered PI Domains

| | PI Start | PI Stop (AA | | |
|---|---|---|---|---|
| | (AA pos) | pos) | | |
| | Start and Stop numbers | | | |
| | refer to the sequences in | | Length of PI | RKR |
| Strain Name | Table 100 | | (AA) | motif (AA) |
| Alicycliphilus | 837 | 1029 | 193 | --Y |
| denitrificans | | | | |
| K601 | | | | |
| Campylobacter | 741 | 984 | 244 | -NG |
| jejuni NCTC | | | | |
| 11168 | | | | |
| Helicobacter | 771 | 1024 | 254 | -NQ |
| mustelae 12198 | | | | |

TABLE 12
Other Altered PI Domains

| | PI Start | PI Stop (AA | | |
|---|---|---|---|---|
| | (AA pos) | pos) | | |
| | Start and Stop numbers | | | |
| | refer to the sequences in | | Length of PI | |
| Strain Name | Table 7 | | (AA) | RKR motif (AA) |
| Akkermansia muciniphila ATCC BAA-835 | 871 | 1101 | 231 | ALK |
| Ralstonia syzygii R24 | 821 | 1062 | 242 | APY |
| Cand. Puniceispirillum marinum IMCC1322 | 815 | 1035 | 221 | AYK |
| Fructobacillus fructosus KCTC 3544 | 1074 | 1323 | 250 | DGN |
| Eubacterium yurii ATCC 43715 | 1107 | 1391 | 285 | DGY |
| Eubacterium dolichum DSM 3991 | 779 | 1096 | 318 | DKK |
| Dinoroseobacter shibae DFL 12 | 851 | 1079 | 229 | DPI |
| Clostridium cellulolyticum H10 | 767 | 1021 | 255 | EGK |
| Pasteurella multocida str. Pm70 | 815 | 1056 | 242 | ENN |
| Mycoplasma canis PG 14 | 907 | 1233 | 327 | EPK |
| Porphyromonas sp. oral taxon 279 str. F0450 | 935 | 1197 | 263 | EPT |
| Filifactor alocis ATCC 35896 | 1094 | 1365 | 272 | EVD |
| Aminomonas paucivorans DSM 12260 | 801 | 1052 | 252 | EVY |
| Wolinella succinogenes DSM 1740 | 1034 | 1409 | 376 | EYK |
| Oenococcus kitaharae DSM 17330 | 1119 | 1389 | 271 | GAL |
| Coriobacterium glomerans PW2 | 1126 | 1384 | 259 | GDR |
| Peptoniphilus duerdenii ATCC BAA-1640 | 1091 | 1364 | 274 | GDS |
| Bifidobacterium bifidum S17 | 1138 | 1420 | 283 | GGL |
| Alicyclobacillus hesperidum URH17-3-68 | 876 | 1146 | 271 | GGR |
| Roseburia inulinivorans DSM 16841 | 895 | 1152 | 258 | GGT |
| Actinomyces coleocanis DSM 15436 | 843 | 1105 | 263 | GKK |
| Odoribacter laneus YIT 12061 | 1103 | 1498 | 396 | GKV |
| Coprococcus catus GD-7 | 1063 | 1338 | 276 | GNQ |
| Enterococcus faecalis TX0012 | 829 | 1150 | 322 | GRK |
| Bacillus smithii 7 3 47FAA | 809 | 1088 | 280 | GSK |
| Legionella pneumophila str. Paris | 1021 | 1372 | 352 | GTM |
| Bacteroides fragilis NCTC 9343 | 1140 | 1436 | 297 | IPV |
| Mycoplasma ovipneumoniae SC01 | 923 | 1265 | 343 | IRI |
| Actinomyces sp. oral taxon 180 str. F0310 | 895 | 1181 | 287 | KEK |
| Treponema sp. JC4 | 832 | 1062 | 231 | KIS |
| Fusobacteriumnucleatum ATCC49256 | 1073 | 1374 | 302 | KKV |
| Lactobacillus farciminis KCTC 3681 | 1101 | 1356 | 256 | KKV |
| Nitratifractor salsuginis DSM 16511 | 840 | 1132 | 293 | KMR |
| Lactobacillus coryniformis KCTC 3535 | 850 | 1119 | 270 | KNK |
| Mycoplasma mobile 163K | 916 | 1236 | 321 | KNY |
| Flavobacterium branchiophilum FL-15 | 1182 | 1473 | 292 | KQK |
| Prevotella timonensis CRIS 5C-B1 | 957 | 1218 | 262 | KQQ |
| Methylosinus trichosporium OB3b | 830 | 1082 | 253 | KRP |
| Prevotella sp. C561 | 1099 | 1424 | 326 | KRY |
| Mycoplasma gallisepticum str. F | 911 | 1269 | 359 | KTA |
| Lactobacillus rhamnosus GG | 1077 | 1363 | 287 | KYG |
| Wolinella succinogenes DSM 1740 | 811 | 1059 | 249 | LPN |
| Streptococcus thermophilus LMD-9 | 1099 | 1388 | 290 | MLA |
| Treponema denticola ATCC 35405 | 1092 | 1395 | 304 | NDS |
| Bergeyella zoohelcum ATCC 43767 | 1098 | 1415 | 318 | NEK |
| Veillonella atypica ACS-134-V-Col7a | 1107 | 1398 | 292 | NGF |
| Neisseria meningitidis Z2491 | 835 | 1082 | 248 | NHN |
| Ignavibacterium album JCM 16511 | 1296 | 1688 | 393 | NKK |
| Ruminococcus albus 8 | 853 | 1156 | 304 | NNF |
| Streptococcus thermophilus LMD-9 | 811 | 1121 | 311 | NNK |
| Barnesiella intestinihominis YIT 11860 | 871 | 1153 | 283 | NPV |
| Azospirillum sp. B510 | 911 | 1168 | 258 | PFH |
| Rhodospirillum rubrum ATCC 11170 | 863 | 1173 | 311 | PRG |
| Planococcus antarcticus DSM 14505 | 1087 | 1333 | 247 | PYY |
| Staphylococcus pseudintermedius ED99 | 1073 | 1334 | 262 | QIV |
| Alcanivorax sp. W11-5 | 843 | 1113 | 271 | RIE |
| Bradyrhizobium sp. BTAi1 | 811 | 1064 | 254 | RIY |
| Streptococcus pyogenes M1 GAS | 1099 | 1368 | 270 | RKR |
| Streptococcus mutans UA159 | 1078 | 1345 | 268 | RKR |
| Streptococcus Pyogenes | 1099 | 1368 | 270 | RKR |
| Bacteroides sp. 20 3 | 1147 | 1517 | 371 | RNI |
| S. aureus | 772 | 1053 | 282 | RNK |
| Solobacterium moorei F0204 | 1062 | 1327 | 266 | RSG |
| Finegoldia magna ATCC 29328 | 1081 | 1348 | 268 | RTE |
| uncultured delta proteobacterium HF0070 07E19 | 770 | 1011 | 242 | SGG |
| Acidaminococcus sp. D21 | 1064 | 1358 | 295 | SIG |
| Eubacterium rectale ATCC 33656 | 824 | 1114 | 291 | SKK |
| Caenispirillum salinarum AK4 | 1048 | 1442 | 395 | SLV |
| Acidothermus cellulolyticus 11B | 830 | 1138 | 309 | SPS |
| Catenibacterium mitsuokai DSM 15897 | 1068 | 1329 | 262 | SPT |
| Parvibaculum lavamentivorans DS-1 | 827 | 1037 | 211 | TGN |
| Staphylococcus lugdunensis M23590 | 772 | 1054 | 283 | TKK |
| Streptococcus sanguinis SK49 | 1123 | 1421 | 299 | TRM |
| Elusimicrobium minutum Pei191 | 910 | 1195 | 286 | TTG |
| Nitrobacter hamburgensis X14 | 914 | 1166 | 253 | VAY |
| Mycoplasma synoviae 53 | 991 | 1314 | 324 | VGF |
| Sphaerochaeta globus str. Buddy | 877 | 1179 | 303 | VKG |
| Ilyobacter polytropus DSM 2926 | 837 | 1092 | 256 | VNG |
| Rhodovulum sp. PH10 | 821 | 1059 | 239 | VPY |
| Bifidobacterium longum DJO10A | 904 | 1187 | 284 | VRK |

### Amino Acid Sequences Described in Table 8:

SEQ ID NO: 304
MKRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEEDT
GNELSTKEQISRNSKALEEKYVAELQLERLKKDGEVRGSINRFKTSDYVKEAKQLLKVQKAYHQ
LDQSFIDTYIDLLETRRTYYEGPGEGSPFGWKDIKEWYEMLMGHCTYFPEELRSVKYAYNADLY
NALNDLNNLVITRDENEKLEYYEKFQIIENVFKQKKKPTLKQIAKEILVNEEDIKGYRVTSTGK
PEFTNLKVYHDIKDITARKEIIENAELLDQIAKILTIYQSSEDIQEELTNLNSELTQEEIEQIS
NLKGYTGTHNLSLKAINLILDELWHTNDNQIAIFNRLKLVPKKVDLSQQKEIPTTLVDDFILSP
VVKRSFIQSIKVINAIIKKYGLPNDIIIELAREKNSKDAQKMINEMQKRNRQTNERIEEIIRTT
GKENAKYLIEKIKLHDMQEGKCLYSLEAIPLEDLLNNPFNYEVDHIIPRSVSFDNSFNNKVLVK
QEENSKKGNRTPFQYLSSSDSKISYETFKKHILNLAKGKGRISKTKKEYLLEERDINRFSVQKD
FINRNLVDTRYATRGLMNLLRSYFRVNNLDVKVKSINGGFTSFLRRKWKFKKERNKGYKHHAED
ALIIANADFIFKEWKKLDKAKKVMENQMFEEKQAESMPEIETEQEYKEIFITPHQIKHIKDFKD
YKYSHRVDKKPNRELINDTLYSTRKDDKGNTLIVNNLNGLYDKDNDKLKKLINKSPEKLLMYHH
DPQTYQKLKLIMEQYGDEKNPLYKYYEETGNYLTKYSKKDNGPVIKKIKYYGNKLNAHLDITDD
YPNSRNKVVKLSLKPYRFDVYLDNGVYKFVTVKNLDVIKKENYYEVNSKCYEEAKKLKKISNQA
EFIASFYNNDLIKINGELYRVIGVNNDLLNRIEVNMIDITYREYLENMNDKRPPRIIKTIASKT
QSIKKYSTDILGNLYEVKSKKHPQIIKKG
SEQ ID NO: 305
MDKKYSIGLDIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD
SEQ ID NO: 306
MARILAFDIGISSIGWAFSENDELKDCGVRIFTKVENPKTGESLALPRRLARSARKRLARRKAR
LNHLKHLIANEFKLNYEDYQSFDESLAKAYKGSLISPYELRFRALNELLSKQDFARVILHIAKR
RGYDDIKNSDDKEKGAILKAIKQNEEKLANYQSVGEYLYKEYFQKFKENSKEFTNVRNKKESYE
RCIAQSFLKDELKLIFKKQREFGFSFSKKFEEEVLSVAFYKRALKDFSHLVGNCSFFTDEKRAP
KNSPLAFMFVALTRIINLLNNLKNTEGILYTKDDLNALLNEVLKNGTLTYKQTKKLLGLSDDYE
FKGEKGTYFIEFKKYKEFIKALGEHNLSQDDLNEIAKDITLIKDEIKLKKALAKYDLNQNQIDS
LSKLEFKDHLNISFKALKLVTPLMLEGKKYDEACNELNLKVAINEDKKDFLPAFNETYYKDEVT
NPVVLRAIKEYRKVLNALLKKYGKVHKINIELAREVGKNHSQRAKIEKEQNENYKAKKDAELEC
EKLGLKINSKNILKLRLFKEQKEFCAYSGEKIKISDLQDEKMLEIDHIYPYSRSFDDSYMNKVL
VFTKQNQEKLNQTPFEAFGNDSAKWQKIEVLAKNLPTKKQKRILDKNYKDKEQKNFKDRNLNDT
RYIARLVLNYTKDYLDFLPLSDDENTKLNDTQKGSKVHVEAKSGMLTSALRHTWGFSAKDRNNH
LHHAIDAVIIAYANNSIVKAFSDFKKEQESNSAELYAKKISELDYKNKRKFFEPFSGFRQKVLD
KIDEIFVSKPERKKPSGALHEETFRKEEEFYQSYGGKEGVLKALELGKIRKVNGKIVKNGDMFR
VDIFKHKKTNKFYAVPIYTMDFALKVLPNKAVARSKKGEIKDWILMDENYEFCFSLYKDSLILI
QTKDMQEPEFVYYNAFTSSTVSLIVSKHDNKFETLSKNQKILFKNANEKEVIAKSIGIQNLKVF
EKYIVSALGEVTKAEFRQREDFKK
SEQ ID NO: 307
MKRILGLDLGTNSIGWALVNEAENKDERSSIVKLGVRVNPLTVDELTNFEKGKSITTNADRTLK
RGMRRNLQRYKLRRETLTEVLKEHKLITEDTILSENGNRTTFETYRLRAKAVTEEISLEEFARV
LLMINKKRGYKSSRKAKGVEEGTLIDGMDIARELYNNNLTPGELCLQLLDAGKKFLPDFYRSDL
QNELDRIWEKQKEYYPEILTDVLKEELRGKKRDAVWAICAKYFVWKENYTEWNKEKGKTEQQER
EHKLEGIYSKRKRDEAKRENLQWRVNGLKEKLSLEQLVIVFQEMNTQINNSSGYLGAISDRSKE
LYFNKQTVGQYQMEMLDKNPNASLRNMVFYRQDYLDEFNMLWEKQAVYHKELTEELKKEIRDII
IFYQRRLKSQKGLIGFCEFESRQIEVDIDGKKKIKTVGNRVISRSSPLFQEFKIWQILNNIEVT
VVGKKRKRRKLKENYSALFEELNDAEQLELNGSRRLCQEEKELLAQELFIRDKMTKSEVLKLLF
DNPQELDLNFKTIDGNKTGYALFQAYSKMIEMSGHEPVDFKKPVEKVVEYIKAVFDLLNWNTDI
LGFNSNEELDNQPYYKLWHLLYSFEGDNTPTGNGRLIQKMTELYGFEKEYATILANVSFQDDYG
SLSAKAIHKILPHLKEGNRYDVACVYAGYRHSESSLTREEIANKVLKDRLMLLPKNSLHNPVVE
KILNQMVNVINVIIDIYGKPDEIRVELARELKKNAKEREELTKSIAQTTKAHEEYKTLLQTEFG
LTNVSRTDILRYKLYKELESCGYKTLYSNTYISREKLFSKEFDIEHIIPQARLFDDSFSNKTLE
ARSVNIEKGNKTAYDFVKEKFGESGADNSLEHYLNNIEDLFKSGKISKTKYNKLKMAEQDIPDG
FIERDLRNTQYIAKKALSMLNEISHRVVATSGSVTDKLREDWQLIDVMKELNWEKYKALGLVEY
FEDRDGRQIGRIKDWTKRNDHRHHAMDALTVAFTKDVFIQYFNNKNASLDPNANEHAIKNKYFQ
NGRAIAPMPLREFRAEAKKHLENTLISIKAKNKVITGNINKTRKKGGVNKNMQQTPRGQLHLET
IYGSGKQYLTKEEKVNASFDMRKIGTVSKSAYRDALLKRLYENDNDPKKAFAGKNSLDKQPIWL
DKEQMRKVPEKVKIVTLEAIYTIRKEISPDLKVDKVIDVGVRKILIDRLNEYGNDAKKAFSNLD
KNPIWLNKEKGISIKRVTISGISNAQSLHVKKDKDGKPILDENGRNIPVDFVNTGNNHHVAVYY
RPVIDKRGQLVVDEAGNPKYELEEVVVSFFEAVTRANLGLPIIDKDYKTTEGWQFLFSMKQNEY
FVFPNEKTGFNPKEIDLLDVENYGLISPNLFRVQKFSLKNYVFRHHLETTIKDTSSILRGITWI
DFRSSKGLDTIVKVRVNHIGQIVSVGEY
SEQ ID NO: 308
MSRKNYVDDYAISLDIGNASVGWSAFTPNYRLVRAKGHELIGVRLFDPADTAESRRMARTTRRR
YSRRRWRLRLLDALFDQALSEIDPSFLARRKYSWVHPDDENNADCWYGSVLFDSNEQDKRFYEK
YPTIYHLRKALMEDDSQHDIREIYLAIHHMVKYRGNFLVEGTLESSNAFKEDELLKLLGRITRY
EMSEGEQNSDIEQDDENKLVAPANGQLADALCATRGSRSMRVDNALEALSAVNDLSREQRAIVK
AIFAGLEGNKLDLAKIFVSKEFSSENKKILGIYFNKSDYEEKCVQIVDSGLLDDEEREFLDRMQ
GQYNAIALKQLLGRSTSVSDSKCASYDAHRANWNLIKLQLRTKENEKDINENYGILVGWKIDSG
QRKSVRGESAYENMRKKANVFFKKMIETSDLSETDKNRLIHDIEEDKLFPIQRDSDNGVIPHQL
HQNELKQIIKKQGKYYPFLLDAFEKDGKQINKIEGLLTFRVPYFVGPLVVPEDLQKSDNSENHW
MVRKKKGEITPWNFDEMVDKDASGRKFIERLVGTDSYLLGEPTLPKNSLLYQEYEVLNELNNVR
LSVRTGNHWNDKRRMRLGREEKTLLCQRLFMKGQTVTKRTAENLLRKEYGRTYELSGLSDESKF
TSSLSTYGKMCRIFGEKYVNEHRDLMEKIVELQTVFEDKETLLHQLRQLEGISEADCALLVNTH
YTGWGRLSRKLLTTKAGECKISDDFAPRKHSIIEIMRAEDRNLMEIITDKQLGFSDWIEQENLG
AENGSSLMEVVDDLRVSPKVKRGIIQSIRLIDDISKAVGKRPSRIFLELADDIQPSGRTISRKS
RLQDLYRNANLGKEFKGIADELNACSDKDLQDDRLFLYYTQLGKDMYTGEELDLDRLSSAYDID
HIIPQAVTQNDSIDNRVLVARAENARKTDSFTYMPQIADRMRNFWQILLDNGLISRVKFERLTR
QNEFSEREKERFVQRSLVETRQIMKNVATLMRQRYGNSAAVIGLNAELTKEMHRYLGFSHKNRD
INDYHHAQDALCVGIAGQFAANRGFFADGEVSDGAQNSYNQYLRDYLRGYREKLSAEDRKQGRA
FGFIVGSMRSQDEQKRVNPRTGEVVWSEEDKDYLRKVMNYRKMLVTQKVGDDFGALYDETRYAA
TDPKGIKGIPFDGAKQDTSLYGGFSSAKPAYAVLIESKGKTRLVNVTMQEYSLLGDRPSDDELR
KVLAKKKSEYAKANILLRHVPKMQLIRYGGGLMVIKSAGELNNAQQLWLPYEEYCYFDDLSQGK
GSLEKDDLKKLLDSILGSVQCLYPWHRFTEEELADLHVAFDKLPEDEKKNVITGIVSALHADAK
TANLSIVGMTGSWRRMNNKSGYTFSDEDEFIFQSPSGLFEKRVTVGELKRKAKKEVNSKYRTNE
KRLPTLSGASQP
SEQ ID NO: 309
METQTSNQLITSHLKDYPKQDYFVGLDIGTNSVGWAVTNTSYELLKFHSHKMWGSRLFEEGESA
VTRRGFRSMRRRLERRKLRLKLLEELFADAMAQVDSTFFIRLHESKYHYEDKTTGHSSKHILFI
DEDYTDQDYFTEYPTIYHLRKDLMENGTDDIRKLFLAVHHILKYRGNFLYEGATFNSNAFTFED
VLKQALVNITFNCFDTNSAISSISNILMESGKTKSDKAKAIERLVDTYTVFDEVNTPDKPQKEQ
VKEDKKTLKAFANLVLGLSANLIDLFGSVEDIDDDLKKLQIVGDTYDEKRDELAKVWGDEIHII
DDCKSVYDAIILMSIKEPGLTISQSKVKAFDKHKEDLVILKSLLKLDRNVYNEMFKSDKKGLHN
YVHYIKQGRTEETSCSREDFYKYTKKIVEGLADSKDKEYILNEIELQTLLPLQRIKDNGVIPYQ
LHLEELKVILDKCGPKFPFLHTVSDGFSVTEKLIKMLEFRIPYYVGPLNTHHNIDNGGFSWAVR
KQAGRVTPWNFEEKIDREKSAAAFIKNLTNKCTYLFGEDVLPKSSLLYSEFMLLNELNNVRIDG
KALAQGVKQHLIDSIFKQDHKKMTKNRIELFLKDNNYITKKHKPEITGLDGEIKNDLTSYRDMV
RILGNNFDVSMAEDIITDITIFGESKKMLRQTLRNKFGSQLNDETIKKLSKLRYRDWGRLSKKL
LKGIDGCDKAGNGAPKTIIELMRNDSYNLMEILGDKFSFMECIEEENAKLAQGQVVNPHDIIDE
LALSPAVKRAVWQALRIVDEVAHIKKALPSRIFVEVARTNKSEKKKKDSRQKRLSDLYSAIKKD
DVLQSGLQDKEFGALKSGLANYDDAALRSKKLYLYYTQMGRCAYTGNIIDLNQLNTDNYDIDHI
YPRSLTKDDSFDNLVLCERTANAKKSDIYPIDNRIQTKQKPFWAFLKHQGLISERKYERLTRIA
PLTADDLSGFIARQLVETNQSVKATTTLLRRLYPDIDVVFVKAENVSDFRHNNNFIKVRSLNHH
HHAKDAYLNIVVGNVYHEKFTRNFRLFFKKNGANRTYNLAKMFNYDVICTNAQDGKAWDVKTSM
NTVKKMMASNDVRVTRRLLEQSGALADATIYKASVAAKAKDGAYIGMKTKYSVFADVTKYGGMT
KIKNAYSIIVQYTGKKGEEIKEIVPLPIYLINRNATDIELIDYVKSVIPKAKDISIKYRKLCIN
QLVKVNGFYYYLGGKTNDKIYIDNAIELVVPHDIATYIKLLDKYDLLRKENKTLKASSITTSIY
NINTSTVVSLNKVGIDVFDYFMSKLRTPLYMKMKGNKVDELSSTGRSKFIKMTLEEQSIYLLEV
LNLLTNSKTTFDVKPLGITGSRSTIGVKIHNLDEFKIINESITGLYSNEVTIV
SEQ ID NO: 310
MTKLNQPYGIGLDIGSNSIGFAVVDANSHLLRLKGETAIGARLFREGQSAADRRGSRTTRRRLS
RTRWRLSFLRDFFAPHITKIDPDFFLRQKYSEISPKDKDRFKYEKRLFNDRTDAEFYEDYPSMY
HLRLHLMTHTHKADPREIFLAIHHILKSRGHFLTPGAAKDFNTDKVDLEDIFPALTEAYAQVYP
DLELTFDLAKADDFKAKLLDEQATPSDTQKALVNLLLSSDGEKEIVKKRKQVLTEFAKAITGLK
TKFNLALGTEVDEADASNWQFSMGQLDDKWSNIETSMTDQGTEIFEQIQELYRARLLNGIVPAG
MSLSQAKVADYGQHKEDLELFKTYLKKLNDHELAKTIRGLYDRYINGDDAKPFLREDFVKALTK
EVTAHPNEVSEQLLNRMGQANFMLKQRTKANGAIPIQLQQRELDQIIANQSKYYDWLAAPNPVE
AHRWKMPYQLDELLNFHIPYYVGPLITPKQQAESGENVFAWMVRKDPSGNITPYNFDEKVDREA
SANTFIQRMKTTDTYLIGEDVLPKQSLLYQKYEVLNELNNVRINNECLGTDQKQRLIREVFERH
SSVTIKQVADNLVAHGDFARRPEIRGLADEKRFLSSLSTYHQLKEILHEAIDDPTKLLDIENII
TWSTVFEDHTIFETKLAEIEWLDPKKINELSGIRYRGWGQFSRKLLDGLKLGNGHTVIQELMLS
NHNLMQILADETLKETMTELNQDKLKTDDIEDVINDAYTSPSNKKALRQVLRVVEDIKHAANGQ
DPSWLFIETADGTGTAGKRTQSRQKQIQTVYANAAQELIDSAVRGELEDKIADKASFTDRLVLY
FMQGGRDIYTGAPLNIDQLSHYDIDHILPQSLIKDDSLDNRVLVNATINREKNNVFASTLFAGK
MKATWRKWHEAGLISGRKLRNLMLRPDEIDKFAKGFVARQLVETRQIIKLTEQIAAAQYPNTKI
IAVKAGLSHQLREELDFPKNRDVNHYHHAFDAFLAARIGTYLLKRYPKLAPFFTYGEFAKVDVK
KFREFNFIGALTHAKKNIIAKDTGEIVWDKERDIRELDRIYNFKRMLITHEVYFETADLFKQTI
YAAKDSKERGGSKQLIPKKQGYPTQVYGGYTQESGSYNALVRVAEADTTAYQVIKISAQNASKI
ASANLKSREKGKQLLNEIVVKQLAKRRKNWKPSANSFKIVIPRFGMGTLFQNAKYGLFMVNSDT
YYRNYQELWLSRENQKLLKKLFSIKYEKTQMNHDALQVYKAIIDQVEKFFKLYDINQFRAKLSD
AIERFEKLPINTDGNKIGKTETLRQILIGLQANGTRSNVKNLGIKTDLGLLQVGSGIKLDKDTQ
IVYQSPSGLFKRRIPLADL
SEQ ID NO: 311
MTKEYYLGLDVGTNSVGWAVTDSQYNLCKFKKKDMWGIRLFESANTAKDRRLQRGNRRRLERKK
QRIDLLQEIFSPEICKIDPTFFIRLNESRLHLEDKSNDFKYPLFIEKDYSDIEYYKEFPTIFHL
RKHLIESEEKQDIRLIYLALHNIIKTRGHFLIDGDLQSAKQLRPILDTFLLSLQEEQNLSVSLS
ENQKDEYEEILKNRSIAKSEKVKKLKNLFEISDELEKEEKKAQSAVIENFCKFIVGNKGDVCKF
LRVSKEELEIDSFSFSEGKYEDDIVKNLEEKVPEKVYLFEQMKAMYDWNILVDILETEEYISFA
KVKQYEKHKTNLRLLRDIILKYCTKDEYNRMFNDEKEAGSYTAYVGKLKKNNKKYWIEKKRNPE
EFYKSLGKLLDKIEPLKEDLEVLTMMIEECKNHTLLPIQKNKDNGVIPHQVHEVELKKILENAK
KYYSFLTETDKDGYSVVQKIESIFRFRIPYYVGPLSTRHQEKGSNVWMVRKPGREDRIYPWNME
EIIDFEKSNENFITRMTNKCTYLIGEDVLPKHSLLYSKYMVLNELNNVKVRGKKLPTSLKQKVF
EDLFENKSKVTGKNLLEYLQIQDKDIQIDDLSGFDKDFKTSLKSYLDFKKQIFGEEIEKESIQN
MIEDIIKWITIYGNDKEMLKRVIRANYSNQLTEEQMKKITGFQYSGWGNFSKMFLKGISGSDVS
TGETFDIITAMWETDNNLMQILSKKFTFMDNVEDFNSGKVGKIDKITYDSTVKEMFLSPENKRA
VWQTIQVAEEIKKVMGCEPKKIFIEMARGGEKVKKRTKSRKAQLLELYAACEEDCRELIKEIED
RDERDFNSMKLFLYYTQFGKCMYSGDDIDINELIRGNSKWDRDHIYPQSKIKDDSIDNLVLVNK
TYNAKKSNELLSEDIQKKMHSFWLSLLNKKLITKSKYDRLTRKGDFTDEELSGFIARQLVETRQ
STKAIADIFKQIYSSEVVYVKSSLVSDFRKKPLNYLKSRRVNDYHHAKDAYLNIVVGNVYNKKF
TSNPIQWMKKNRDTNYSLNKVFEHDVVINGEVIWEKCTYHEDTNTYDGGTLDRIRKIVERDNIL
YTEYAYCEKGELFNATIQNKNGNSTVSLKKGLDVKKYGGYFSANTSYFSLIEFEDKKGDRARHI
IGVPIYIANMLEHSPSAFLEYCEQKGYQNVRILVEKIKKNSLLIINGYPLRIRGENEVDTSFKR
AIQLKLDQKNYELVRNIEKFLEKYVEKKGNYPIDENRDHITHEKMNQLYEVLLSKMKKFNKKGM
ADPSDRIEKSKPKFIKLEDLIDKINVINKMLNLLRCDNDTKADLSLIELPKNAGSFVVKKNTIG
KSKIILVNQSVTGLYENRREL
SEQ ID NO: 312
MARDYSVGLDIGTSSVGWAAIDNKYHLIRAKSKNLIGVRLFDSAVTAEKRRGYRTTRRRLSRRH
WRLRLLNDIFAGPLTDFGDENFLARLKYSWVHPQDQSNQAHFAAGLLFDSKEQDKDFYRKYPTI
YHLRLALMNDDQKHDLREVYLAIHHLVKYRGHFLIEGDVKADSAFDVHTFADAIQRYAESNNSD
ENLLGKIDEKKLSAALTDKHGSKSQRAETAETAFDILDLQSKKQIQAILKSVVGNQANLMAIFG
LDSSAISKDEQKNYKFSFDDADIDEKIADSEALLSDTEFEFLCDLKAAFDGLTLKMLLGDDKTV
SAAMVRRFNEHQKDWEYIKSHIRNAKNAGNGLYEKSKKFDGINAAYLALQSDNEDDRKKAKKIF
QDEISSADIPDDVKADFLKKIDDDQFLPIQRTKNNGTIPHQLHRNELEQIIEKQGIYYPFLKDT
YQENSHELNKITALINFRVPYYVGPLVEEEQKIADDGKNIPDPTNHWMVRKSNDTITPWNLSQV
VDLDKSGRRFIERLTGTDTYLIGEPTLPKNSLLYQKFDVLQELNNIRVSGRRLDIRAKQDAFEH
LFKVQKTVSATNLKDFLVQAGYISEDTQIEGLADVNGKNFNNALTTYNYLVSVLGREFVENPSN
EELLEEITELQTVFEDKKVLRRQLDQLDGLSDHNREKLSRKHYTGWGRISKKLLTTKIVQNADK
IDNQTFDVPRMNQSIIDTLYNTKMNLMEIINNAEDDFGVRAWIDKQNTTDGDEQDVYSLIDELA
GPKEIKRGIVQSFRILDDITKAVGYAPKRVYLEFARKTQESHLTNSRKNQLSTLLKNAGLSELV
TQVSQYDAAALQNDRLYLYFLQQGKDMYSGEKLNLDNLSNYDIDHIIPQAYTKDNSLDNRVLVS
NITNRRKSDSSNYLPALIDKMRPFWSVLSKQGLLSKHKFANLTRTRDFDDMEKERFIARSLVET
RQIIKNVASLIDSHFGGETKAVAIRSSLTADMRRYVDIPKNRDINDYHHAFDALLFSTVGQYTE
NSGLMKKGQLSDSAGNQYNRYIKEWIHAARLNAQSQRVNPFGFVVGSMRNAAPGKLNPETGEIT
PEENADWSIADLDYLHKVMNFRKITVTRRLKDQKGQLYDESRYPSVLHDAKSKASINFDKHKPV
DLYGGFSSAKPAYAALIKFKNKFRLVNVLRQWTYSDKNSEDYILEQIRGKYPKAEMVLSHIPYG
QLVKKDGALVTISSATELHNFEQLWLPLADYKLINTLLKTKEDNLVDILHNRLDLPEMTIESAF
YKAFDSILSFAFNRYALHQNALVKLQAHRDDFNALNYEDKQQTLERILDALHASPASSDLKKIN
LSSGFGRLFSPSHFTLADTDEFIFQSVTGLFSTQKTVAQLYQETK
SEQ ID NO: 313
MVYDVGLDIGTGSVGWVALDENGKLARAKGKNLVGVRLFDTAQTAADRRGFRTTRRRLSRRKWR
LRLLDELFSAEINEIDSSFFQRLKYSYVHPKDEENKAHYYGGYLFPTEEETKKFHRSYPTIYHL
RQELMAQPNKRFDIREIYLAIHHLVKYRGHFLSSQEKITIGSTYNPEDLANAIEVYADEKGLSW
ELNNPEQLTEIISGEAGYGLNKSMKADEALKLFEFDNNQDKVAIKTLLAGLTGNQIDFAKLFGK
DISDKDEAKLWKLKLDDEALEEKSQTILSQLTDEEIELFHAVVQAYDGFVLIGLLNGADSVSAA
MVQLYDQHREDRKLLKSLAQKAGLKHKRFSEIYEQLALATDEATIKNGISTARELVEESNLSKE
VKEDTLRRLDENEFLPKQRTKANSVIPHQLHLAELQKILQNQGQYYPFLLDTFEKEDGQDNKIE
ELLRFRIPYYVGPLVTKKDVEHAGGDADNHWVERNEGFEKSRVTPWNFDKVFNRDKAARDFIER
LTGNDTYLIGEKTLPQNSLRYQLFTVLNELNNVRVNGKKFDSKTKADLINDLFKARKTVSLSAL
KDYLKAQGKGDVTITGLADESKFNSSLSSYNDLKKTFDAEYLENEDNQETLEKIIEIQTVFEDS
KIASRELSKLPLDDDQVKKLSQTHYTGWGRLSEKLLDSKIIDERGQKVSILDKLKSTSQNFMSI
INNDKYGVQAWITEQNTGSSKLTFDEKVNELTTSPANKRGIKQSFAVLNDIKKAMKEEPRRVYL
EFAREDQTSVRSVPRYNQLKEKYQSKSLSEEAKVLKKTLDGNKNKMSDDRYFLYFQQQGKDMYT
GRPINFERLSQDYDIDHIIPQAFTKDDSLDNRVLVSRPENARKSDSFAYTDEVQKQDGSLWTSL
LKSGFINRKKYERLTKAGKYLDGQKTGFIARQLVETRQIIKNVASLIEGEYENSKAVAIRSEIT
ADMRLLVGIKKHREINSFHHAFDALLITAAGQYMQNRYPDRDSTNVYNEFDRYTNDYLKNLRQL
SSRDEVRRLKSFGFVVGTMRKGNEDWSEENTSYLRKVMMFKNILTTKKTEKDRGPLNKETIFSP
KSGKKLIPLNSKRSDTALYGGYSNVYSAYMTLVRANGKNLLIKIPISIANQIEVGNLKINDYIV
NNPAIKKFEKILISKLPLGQLVNEDGNLIYLASNEYRHNAKQLWLSTTDADKIASISENSSDEE
LLEAYDILTSENVKNRFPFFKKDIDKLSQVRDEFLDSDKRIAVIQTILRGLQIDAAYQAPVKII
SKKVSDWHKLQQSGGIKLSDNSEMIYQSATGIFETRVKISDLL
SEQ ID NO: 314
IVDYCIGLDLGTGSVGWAVVDMNHRLMKRNGKHLWGSRLFSNAETAANRRASRSIRRRYNKRRE
RIRLLRAILQDMVLEKDPTFFIRLEHTSFLDEEDKAKYLGTDYKDNYNLFIDEDFNDYTYYHKY
PTIYHLRKALCESTEKADPRLIYLALHHIVKYRGNFLYEGQKFNMDASNIEDKLSDIFTQFTSF
NNIPYEDDEKKNLEILEILKKPLSKKAKVDEVMTLIAPEKDYKSAFKELVTGIAGNKMNVTKMI
LCEPIKQGDSEIKLKFSDSNYDDQFSEVEKDLGEYVEFVDALHNVYSWVELQTIMGATHTDNAS
ISEAMVSRYNKHHDDLKLLKDCIKNNVPNKYFDMFRNDSEKSKGYYNYINRPSKAPVDEFYKYV
KKCIEKVDTPEAKQILNDIELENFLLKQNSRTNGSVPYQMQLDEMIKIIDNQAEYYPILKEKRE
QLLSILTFRIPYYFGPLNETSEHAWIKRLEGKENQRILPWNYQDIVDVDATAEGFIKRMRSYCT
YFPDEEVLPKNSLIVSKYEVYNELNKIRVDDKLLEVDVKNDIYNELFMKNKTVTEKKLKNWLVN
NQCCSKDAEIKGFQKENQFSTSLTPWIDFTNIFGKIDQSNFDLIENIIYDLTVFEDKKIMKRRL
KKKYALPDDKVKQILKLKYKDWSRLSKKLLDGIVADNRFGSSVTVLDVLEMSRLNLMEIINDKD
LGYAQMIEEATSCPEDGKFTYEEVERLAGSPALKRGIWQSLQIVEEITKVMKCRPKYIYIEFER
SEEAKERTESKIKKLENVYKDLDEQTKKEYKSVLEELKGFDNTKKISSDSLFLYFTQLGKCMYS
GKKLDIDSLDKYQIDHIVPQSLVKDDSFDNRVLVVPSENQRKLDDLVVPFDIRDKMYRFWKLLF
DHELISPKKFYSLIKTEYTERDEERFINRQLVETRQITKNVTQIIEDHYSTTKVAAIRANLSHE
FRVKNHIYKNRDINDYHHAHDAYIVALIGGFMRDRYPNMHDSKAVYSEYMKMFRKNKNDQKRWK
DGFVINSMNYPYEVDGKLIWNPDLINEIKKCFYYKDCYCTTKLDQKSGQLFNLTVLSNDAHADK
GVTKAVVPVNKNRSDVHKYGGFSGLQYTIVAIEGQKKKGKKTELVKKISGVPLHLKAASINEKI
NYIEEKEGLSDVRIIKDNIPVNQMIEMDGGEYLLTSPTEYVNARQLVLNEKQCALIADIYNAIY
KQDYDNLDDILMIQLYIELTNKMKVLYPAYRGIAEKFESMNENYVVISKEEKANIIKQMLIVMH
RGPQNGNIVYDDFKISDRIGRLKTKNHNLNNIVFISQSPTGIYTKKYKL
SEQ ID NO: 315
MKSEKKYYIGLDVGTNSVGWAVTDEFYNILRAKGKDLWGVRLFEKADTAANTRIFRSGRRRNDR
KGMRLQILREIFEDEIKKVDKDFYDRLDESKFWAEDKKVSGKYSLFNDKNFSDKQYFEKFPTIF
HLRKYLMEEHGKVDIRYYFLAINQMMKRRGHFLIDGQISHVTDDKPLKEQLILLINDLLKIELE
EELMDSIFEILADVNEKRTDKKNNLKELIKGQDFNKQEGNILNSIFESIVTGKAKIKNIISDED
ILEKIKEDNKEDFVLTGDSYEENLQYFEEVLQENITLFNTLKSTYDFLILQSILKGKSTLSDAQ
VERYDEHKKDLEILKKVIKKYDEDGKLFKQVFKEDNGNGYVSYIGYYLNKNKKITAKKKISNIE
FTKYVKGILEKQCDCEDEDVKYLLGKIEQENFLLKQISSINSVIPHQIHLFELDKILENLAKNY
PSFNNKKEEFTKIEKIRKTFTFRIPYYVGPLNDYHKNNGGNAWIFRNKGEKIRPWNFEKIVDLH
KSEEEFIKRMLNQCTYLPEETVLPKSSILYSEYMVLNELNNLRINGKPLDTDVKLKLIEELFKK
KTKVTLKSIRDYMVRNNFADKEDFDNSEKNLEIASNMKSYIDFNNILEDKFDVEMVEDLIEKIT
IHTGNKKLLKKYIEETYPDLSSSQIQKIINLKYKDWGRLSRKLLDGIKGTKKETEKTDTVINFL
RNSSDNLMQIIGSQNYSFNEYIDKLRKKYIPQEISYEVVENLYVSPSVKKMIWQVIRVTEEITK
VMGYDPDKIFIEMAKSEEEKKTTISRKNKLLDLYKAIKKDERDSQYEKLLTGLNKLDDSDLRSR
KLYLYYTQMGRDMYTGEKIDLDKLFDSTHYDKDHIIPQSMKKDDSIINNLVLVNKNANQTTKGN
IYPVPSSIRNNPKIYNYWKYLMEKEFISKEKYNRLIRNTPLTNEELGGFINRQLVETRQSTKAI
KELFEKFYQKSKIIPVKASLASDLRKDMNTLKSREVNDLHHAHDAFLNIVAGDVWNREFTSNPI
NYVKENREGDKVKYSLSKDFTRPRKSKGKVIWTPEKGRKLIVDTLNKPSVLISNESHVKKGELF
NATIAGKKDYKKGKIYLPLKKDDRLQDVSKYGGYKAINGAFFFLVEHTKSKKRIRSIELFPLHL
LSKFYEDKNTVLDYAINVLQLQDPKIIIDKINYRTEIIIDNFSYLISTKSNDGSITVKPNEQMY
WRVDEISNLKKIENKYKKDAILTEEDRKIMESYIDKIYQQFKAGKYKNRRTTDTIIEKYEIIDL
DTLDNKQLYQLLVAFISLSYKTSNNAVDFTVIGLGTECGKPRITNLPDNTYLVYKSITGIYEKR
IRIK
SEQ ID NO: 316
MKLRGIEDDYSIGLDMGTSSVGWAVTDERGTLAHFKRKPTWGSRLFREAQTAAVARMPRGQRRR
YVRRRWRLDLLQKLFEQQMEQADPDFFIRLRQSRLLRDDRAEEHADYRWPLFNDCKFTERDYYQ
RFPTIYHVRSWLMETDEQADIRLIYLALHNIVKHRGNFLREGQSLSAKSARPDEALNHLRETLR
VWSSERGFECSIADNGSILAMLTHPDLSPSDRRKKIAPLFDVKSDDAAADKKLGIALAGAVIGL
KTEFKNIFGDFPCEDSSIYLSNDEAVDAVRSACPDDCAELFDRLCEVYSAYVLQGLLSYAPGQT
ISANMVEKYRRYGEDLALLKKLVKIYAPDQYRMFFSGATYPGTGIYDAAQARGYTKYNLGPKKS
EYKPSESMQYDDFRKAVEKLFAKTDARADERYRMMMDRFDKQQFLRRLKTSDNGSIYHQLHLEE
LKAIVENQGRFYPFLKRDADKLVSLVSFRIPYYVGPLSTRNARTDQHGENRFAWSERKPGMQDE
PIFPWNWESIIDRSKSAEKFILRMTGMCTYLQQEPVLPKSSLLYEEFCVLNELNGAHWSIDGDD
EHRFDAADREGIIEELFRRKRTVSYGDVAGWMERERNQIGAHVCGGQGEKGFESKLGSYIFFCK
DVFKVERLEQSDYPMIERIILWNTLFEDRKILSQRLKEEYGSRLSAEQIKTICKKRFTGWGRLS
EKFLTGITVQVDEDSVSIMDVLREGCPVSGKRGRAMVMMEILRDEELGFQKKVDDFNRAFFAEN
AQALGVNELPGSPAVRRSLNQSIRIVDEIASIAGKAPANIFIEVTRDEDPKKKGRRTKRRYNDL
KDALEAFKKEDPELWRELCETAPNDMDERLSLYFMQRGKCLYSGRAIDIHQLSNAGIYEVDHII
PRTYVKDDSLENKALVYREENQRKTDMLLIDPEIRRRMSGYWRMLHEAKLIGDKKFRNLLRSRI
DDKALKGFIARQLVETGQMVKLVRSLLEARYPETNIISVKASISHDLRTAAELVKCREANDFHH
AHDAFLACRVGLFIQKRHPCVYENPIGLSQVVRNYVRQQADIFKRCRTIPGSSGFIVNSFMTSG
FDKETGEIFKDDWDAEAEVEGIRRSLNFRQCFISRMPFEDHGVFWDATIYSPRAKKTAALPLKQ
GLNPSRYGSFSREQFAYFFIYKARNPRKEQTLFEFAQVPVRLSAQIRQDENALERYARELAKDQ
GLEFIRIERSKILKNQLIEIDGDRLCITGKEEVRNACELAFAQDEMRVIRMLVSEKPVSRECVI
SLFNRILLHGDQASRRLSKQLKLALLSEAFSEASDNVQRNVVLGLIAIFNGSTNMVNLSDIGGS
KFAGNVRIKYKKELASPKVNVHLIDQSVTGMFERRTKIGL
SEQ ID NO: 317
MENKQYYIGLDVGTNSVGWAVTDTSYNLLRAKGKDMWGARLFEKANTAAERRTKRTSRRRSERE
KARKAMLKELFADEINRVDPSFFIRLEESKFFLDDRSENNRQRYTLFNDATFTDKDYYEKYKTI
FHLRSALINSDEKFDVRLVFLAILNLFSHRGHFLNASLKGDGDIQGMDVFYNDLVESCEYFEIE
LPRITNIDNFEKILSQKGKSRTKILEELSEELSISKKDKSKYNLIKLISGLEASVVELYNIEDI
QDENKKIKIGFRESDYEESSLKVKEIIGDEYFDLVERAKSVHDMGLLSNIIGNSKYLCEARVEA
YENHHKDLLKIKELLKKYDKKAYNDMFRKMTDKNYSAYVGSVNSNIAKERRSVDKRKIEDLYKY
IEDTALKNIPDDNKDKIEILEKIKLGEFLKKQLTASNGVIPNQLQSRELRAILKKAENYLPFLK
EKGEKNLTVSEMIIQLFEFQIPYYVGPLDKNPKKDNKANSWAKIKQGGRILPWNFEDKVDVKGS
RKEFIEKMVRKCTYISDEHTLPKQSLLYEKFMVLNEINNIKIDGEKISVEAKQKIYNDLFVKGK
KVSQKDIKKELISLNIMDKDSVLSGTDTVCNAYLSSIGKFTGVFKEEINKQSIVDMIEDIIFLK
TVYGDEKRFVKEEIVEKYGDEIDKDKIKRILGFKFSNWGNLSKSFLELEGADVGTGEVRSIIQS
LWETNFNLMELLSSRFTYMDELEKRVKKLEKPLSEWTIEDLDDMYLSSPVKRMIWQSMKIVDEI
QTVIGYAPKRIFVEMTRSEGEKVRTKSRKDRLKELYNGIKEDSKQWVKELDSKDESYFRSKKMY
LYYLQKGRCMYSGEVIELDKLMDDNLYDIDHIYPRSFVKDDSLDNLVLVKKEINNRKQNDPITP
QIQASCQGFWKILHDQGFMSNEKYSRLTRKTQEFSDEEKLSFINRQIVETGQATKCMAQILQKS
MGEDVDVVFSKARLVSEFRHKFELFKSRLINDFHHANDAYLNIVVGNSYFVKFTRNPANFIKDA
RKNPDNPVYKYHMDRFFERDVKSKSEVAWIGQSEGNSGTIVIVKKTMAKNSPLITKKVEEGHGS
ITKETIVGVKEIKFGRNKVEKADKTPKKPNLQAYRPIKTSDERLCNILRYGGRTSISISGYCLV
EYVKKRKTIRSLEAIPVYLGRKDSLSEEKLLNYFRYNLNDGGKDSVSDIRLCLPFISTNSLVKI
DGYLYYLGGKNDDRIQLYNAYQLKMKKEEVEYIRKIEKAVSMSKFDEIDREKNPVLTEEKNIEL
YNKIQDKFENTVFSKRMSLVKYNKKDLSFGDFLKNKKSKFEEIDLEKQCKVLYNIIFNLSNLKE
VDLSDIGGSKSTGKCRCKKNITNYKEFKLIQQSITGLYSCEKDLMTI
SEQ ID NO: 318
MKNLKEYYIGLDIGTASVGWAVTDESYNIPKFNGKKMWGVRLFDDAKTAEERRTQRGSRRRLNR
RKERINLLQDLFATEISKVDPNFFLRLDNSDLYREDKDEKLKSKYTLFNDKDFKDRDYHKKYPT
IHHLIMDLIEDEGKKDIRLLYLACHYLLKNRGHFIFEGQKFDTKNSFDKSINDLKIHLRDEYNI
DLEFNNEDLIEIITDTTLNKTNKKKELKNIVGDTKFLKAISAIMIGSSQKLVDLFEDGEFEETT
VKSVDFSTTAFDDKYSEYEEALGDTISLLNILKSIYDSSILENLLKDADKSKDGNKYISKAFVK
KFNKHGKDLKTLKRIIKKYLPSEYANIFRNKSINDNYVAYTKSNITSNKRTKASKFTKQEDFYK
FIKKHLDTIKETKLNSSENEDLKLIDEMLTDIEFKTFIPKLKSSDNGVIPYQLKLMELKKILDN
QSKYYDFLNESDEYGTVKDKVESIMEFRIPYYVGPLNPDSKYAWIKRENTKITPWNFKDIVDLD
SSREEFIDRLIGRCTYLKEEKVLPKASLIYNEFMVLNELNNLKLNEFLITEEMKKAIFEELFKT
KKKVTLKAVSNLLKKEFNLTGDILLSGTDGDFKQGLNSYIDFKNIIGDKVDRDDYRIKIEEIIK
LIVLYEDDKTYLKKKIKSAYKNDFTDDEIKKIAALNYKDWGRLSKRFLTGIEGVDKTTGEKGSI
IYFMREYNLNLMELMSGHYTFTEEVEKLNPVENRELCYEMVDELYLSPSVKRMLWQSLRVVDEI
KRIIGKDPKKIFIEMARAKEAKNSRKESRKNKLLEFYKFGKKAFINEIGEERYNYLLNEINSEE
ESKFRWDNLYLYYTQLGRCMYSLEPIDLADLKSNNIYDQDHIYPKSKIYDDSLENRVLVKKNLN
HEKGNQYPIPEKVLNKNAYGFWKILFDKGLIGQKKYTRLTRRTPFEERELAEFIERQIVETRQA
TKETANLLKNICQDSEIVYSKAENASRFRQEFDIIKCRTVNDLHHMHDAYLNIVVGNVYNTKFT
KNPLNFIKDKDNVRSYNLENMFKYDVVRGSYTAWIADDSEGNVKAATIKKVKRELEGKNYRFTR
MSYIGTGGLYDQNLMRKGKGQIPQKENTNKSNIEKYGGYNKASSAYFALIESDGKAGRERTLET
IPIMVYNQEKYGNTEAVDKYLKDNLELQDPKILKDKIKINSLIKLDGFLYNIKGKTGDSLSIAG
SVQLIVNKEEQKLIKKMDKFLVKKKDNKDIKVTSFDNIKEEELIKLYKTLSDKLNNGIYSNKRN
NQAKNISEALDKFKEISIEEKIDVLNQIILLFQSYNNGCNLKSIGLSAKTGVVFIPKKLNYKEC
KLINQSITGLFENEVDLLNL
SEQ ID NO: 319
MGKMYYLGLDIGTNSVGYAVTDPSYHLLKFKGEPMWGAHVFAAGNQSAERRSFRTSRRRLDRRQ
QRVKLVQEIFAPVISPIDPRFFIRLHESALWRDDVAETDKHIFFNDPTYTDKEYYSDYPTIHHL
IVDLMESSEKHDPRLVYLAVAWLVAHRGHFLNEVDKDNIGDVLSFDAFYPEFLAFLSDNGVSPW
VCESKALQATLLSRNSVNDKYKALKSLIFGSQKPEDNFDANISEDGLIQLLAGKKVKVNKLFPQ
ESNDASFTLNDKEDAIEEILGTLTPDECEWIAHIRRLFDWAIMKHALKDGRTISESKVKLYEQH
HHDLTQLKYFVKTYLAKEYDDIFRNVDSETTKNYVAYSYHVKEVKGTLPKNKATQEEFCKYVLG
KVKNIECSEADKVDFDEMIQRLTDNSFMPKQVSGENRVIPYQLYYYELKTILNKAASYLPFLTQ
CGKDAISNQDKLLSIMTFRIPYFVGPLRKDNSEHAWLERKAGKIYPWNFNDKVDLDKSEEAFIR
RMTNTCTYYPGEDVLPLDSLIYEKFMILNEINNIRIDGYPISVDVKQQVFGLFEKKRRVTVKDI
QNLLLSLGALDKHGKLTGIDTTIHSNYNTYHHFKSLMERGVLTRDDVERIVERMTYSDDTKRVR
LWLNNNYGTLTADDVKHISRLRKHDFGRLSKMFLTGLKGVHKETGERASILDFMWNTNDNLMQL
LSECYTFSDEITKLQEAYYAKAQLSLNDFLDSMYISNAVKRPIYRTLAVVNDIRKACGTAPKRI
FIEMARDGESKKKRSVTRREQIKNLYRSIRKDFQQEVDFLEKILENKSDGQLQSDALYLYFAQL
GRDMYTGDPIKLEHIKDQSFYNIDHIYPQSMVKDDSLDNKVLVQSEINGEKSSRYPLDAAIRNK
MKPLWDAYYNHGLISLKKYQRLTRSTPFTDDEKWDFINRQLVETRQSTKALAILLKRKFPDTEI
VYSKAGLSSDFRHEFGLVKSRNINDLHHAKDAFLAIVTGNVYHERFNRRWFMVNQPYSVKTKTL
FTHSIKNGNFVAWNGEEDLGRIVKMLKQNKNTIHFTRFSFDRKEGLFDIQPLKASTGLVPRKAG
LDVVKYGGYDKSTAAYYLLVRFTLEDKKTQHKLMMIPVEGLYKARIDHDKEFLTDYAQTTISEI
LQKDKQKVINIMFPMGTRHIKLNSMISIDGFYLSIGGKSSKGKSVLCHAMVPLIVPHKIECYIK
AMESFARKFKENNKLRIVEKFDKITVEDNLNLYELFLQKLQHNPYNKFFSTQFDVLTNGRSTFT
KLSPEEQVQTLLNILSIFKTCRSSGCDLKSINGSAQAARIMISADLTGLSKKYSDIRLVEQSAS
GLFVSKSQNLLEYL
SEQ ID NO: 320
MTKKEQPYNIGLDIGTSSVGWAVTNDNYDLLNIKKKNLWGVRLFEEAQTAKETRLNRSTRRRYR
RRKNRINWLNEIFSEELAKTDPSFLIRLQNSWVSKKDPDRKRDKYNLFIDGPYTDKEYYREFPT
IFHLRKELILNKDKADIRLIYLALHNILKYRGNFTYEHQKFNISNLNNNLSKELIELNQQLIKY
DISFPDDCDWNHISDILIGRGNATQKSSNILKDFTLDKETKKLLKEVINLILGNVAHLNTIFKT
SLTKDEEKLNFSGKDIESKLDDLDSILDDDQFTVLDAANRIYSTITLNEILNGESYFSMAKVNQ
YENHAIDLCKLRDMWHTTKNEEAVEQSRQAYDDYINKPKYGTKELYTSLKKFLKVALPTNLAKE
AEEKISKGTYLVKPRNSENGVVPYQLNKIEMEKIIDNQSQYYPFLKENKEKLLSILSFRIPYYV
GPLQSAEKNPFAWMERKSNGHARPWNFDEIVDREKSSNKFIRRMTVTDSYLVGEPVLPKNSLIY
QRYEVLNELNNIRITENLKTNPIGSRLTVETKQRIYNELFKKYKKVTVKKLTKWLIAQGYYKNP
ILIGLSQKDEFNSTLTTYLDMKKIFGSSFMEDNKNYDQIEELIEWLTIFEDKQILNEKLHSSKY
SYTPDQIKKISNMRYKGWGRLSKKILMDITTETNTPQLLQLSNYSILDLMWATNNNFISIMSND
KYDFKNYIENHNLNKNEDQNISDLVNDIHVSPALKRGITQSIKIVQEIVKFMGHAPKHIFIEVT
RETKKSEITTSREKRIKRLQSKLLNKANDFKPQLREYLVPNKKIQEELKKHKNDLSSERIMLYF
LQNGKSLYSEESLNINKLSDYQVDHILPRTYIPDDSLENKALVLAKENQRKADDLLLNSNVIDR
NLERWTYMLNNNMIGLKKFKNLTRRVITDKDKLGFIHRQLVQTSQMVKGVANILDNMYKNQGTT
CIQARANLSTAFRKALSGQDDTYHFKHPELVKNRNVNDFHHAQDAYLASFLGTYRLRRFPTNEM
LLMNGEYNKFYGQVKELYSKKKKLPDSRKNGFIISPLVNGTTQYDRNTGEIIWNVGFRDKILKI
FNYHQCNVTRKTEIKTGQFYDQTIYSPKNPKYKKLIAQKKDMDPNIYGGFSGDNKSSITIVKID
NNKIKPVAIPIRLINDLKDKKTLQNWLEENVKHKKSIQIIKNNVPIGQIIYSKKVGLLSLNSDR
EVANRQQLILPPEHSALLRLLQIPDEDLDQILAFYDKNILVEILQELITKMKKFYPFYKGEREF
LIANIENFNQATTSEKVNSLEELITLLHANSTSAHLIFNNIEKKAFGRKTHGLTLNNTDFIYQS
VTGLYETRIHIE
SEQ ID NO: 321
MTKFNKNYSIGLDIGVSSVGYAVVTEDYRVPAFKFKVLGNTEKEKIKKNLIGSTTFVSAQPAKG
TRVFRVNRRRIDRRNHRITYLRDIFQKEIEKVDKNFYRRLDESFRVLGDKSEDLQIKQPFFGDK
ELETAYHKKYPTIYHLRKHLADADKNSPVADIREVYMAISHILKYRGHFLTLDKINPNNINMQN
SWIDFIESCQEVFDLEISDESKNIADIFKSSENRQEKVKKILPYFQQELLKKDKSIFKQLLQLL
FGLKTKFKDCFELEEEPDLNFSKENYDENLENFLGSLEEDFSDVFAKLKVLRDTILLSGMLTYT
GATHARFSATMVERYEEHRKDLQRFKFFIKQNLSEQDYLDIFGRKTQNGFDVDKETKGYVGYIT
NKMVLTNPQKQKTIQQNFYDYISGKITGIEGAEYFLNKISDGTFLRKLRTSDNGAIPNQIHAYE
LEKIIERQGKDYPFLLENKDKLLSILTFKIPYYVGPLAKGSNSRFAWIKRATSSDILDDNDEDT
RNGKIRPWNYQKLINMDETRDAFITNLIGNDIILLNEKVLPKRSLIYEEVMLQNELTRVKYKDK
YGKAHFFDSELRQNIINGLFKNNSKRVNAKSLIKYLSDNHKDLNAIEIVSGVEKGKSFNSTLKT
YNDLKTIFSEELLDSEIYQKELEEIIKVITVFDDKKSIKNYLTKFFGHLEILDEEKINQLSKLR
YSGWGRYSAKLLLDIRDEDTGFNLLQFLRNDEENRNLTKLISDNTLSFEPKIKDIQSKSTIEDD
IFDEIKKLAGSPAIKRGILNSIKIVDELVQIIGYPPHNIVIEMARENMTTEEGQKKAKTRKTKL
ESALKNIENSLLENGKVPHSDEQLQSEKLYLYYLQNGKDMYTLDKTGSPAPLYLDQLDQYEVDH
IIPYSFLPIDSIDNKVLTHRENNQQKLNNIPDKETVANMKPFWEKLYNAKLISQTKYQRLTTSE
RTPDGVLTESMKAGFIERQLVETRQIIKHVARILDNRFSDTKIITLKSQLITNFRNTFHIAKIR
ELNDYHHAHDAYLAVVVGQTLLKVYPKLAPELIYGHHAHFNRHEENKATLRKHLYSNIMRFFNN
PDSKVSKDIWDCNRDLPIIKDVIYNSQINFVKRTMIKKGAFYNQNPVGKFNKQLAANNRYPLKT
KALCLDTSIYGGYGPMNSALSIIIIAERFNEKKGKIETVKEFHDIFIIDYEKFNNNPFQFLNDT
SENGFLKKNNINRVLGFYRIPKYSLMQKIDGTRMLFESKSNLHKATQFKLTKTQNELFFHMKRL
LTKSNLMDLKSKSAIKESQNFILKHKEEFDNISNQLSAFSQKMLGNTTSLKNLIKGYNERKIKE
IDIRDETIKYFYDNFIKMFSFVKSGAPKDINDFFDNKCTVARMRPKPDKKLLNATLIHQSITGL
YETRIDLSKLGED
SEQ ID NO: 322
MKQEYFLGLDMGTGSLGWAVTDSTYQVMRKHGKALWGTRLFESASTAEERRMFRTARRRLDRRN
WRIQVLQEIFSEEISKVDPGFFLRMKESKYYPEDKRDAEGNCPELPYALFVDDNYTDKNYHKDY
PTIYHLRKMLMETTEIPDIRLVYLVLHHMMKHRGHFLLSGDISQIKEFKSTFEQLIQNIQDEEL
EWHISLDDAAIQFVEHVLKDRNLTRSTKKSRLIKQLNAKSACEKAILNLLSGGTVKLSDIFNNK
ELDESERPKVSFADSGYDDYIGIVEAELAEQYYIIASAKAVYDWSVLVEILGNSVSISEAKIKV
YQKHQADLKTLKKIVRQYMTKEDYKRVFVDTEEKLNNYSAYIGMTKKNGKKVDLKSKQCTQADF
YDFLKKNVIKVIDHKEITQEIESEIEKENFLPKQVTKDNGVIPYQVHDYELKKILDNLGTRMPF
IKENAEKIQQLFEFRIPYYVGPLNRVDDGKDGKFTWSVRKSDARIYPWNFTEVIDVEASAEKFI
RRMTNKCTYLVGEDVLPKDSLVYSKFMVLNELNNLRLNGEKISVELKQRIYEELFCKYRKVTRK
KLERYLVIEGIAKKGVEITGIDGDFKASLTAYHDFKERLTDVQLSQRAKEAIVLNVVLFGDDKK
LLKQRLSKMYPNLTTGQLKGICSLSYQGWGRLSKTFLEEITVPAPGTGEVWNIMTALWQTNDNL
MQLLSRNYGFTNEVEEFNTLKKETDLSYKTVDELYVSPAVKRQIWQTLKVVKEIQKVMGNAPKR
VFVEMAREKQEGKRSDSRKKQLVELYRACKNEERDWITELNAQSDQQLRSDKLFLYYIQKGRCM
YSGETIQLDELWDNTKYDIDHIYPQSKTMDDSLNNRVLVKKNYNAIKSDTYPLSLDIQKKMMSF
WKMLQQQGFITKEKYVRLVRSDELSADELAGFIERQIVETRQSTKAVATILKEALPDTEIVYVK
AGNVSNFRQTYELLKVREMNDLHHAKDAYLNIVVGNAYFVKFTKNAAWFIRNNPGRSYNLKRMF
EFDIERSGEIAWKAGNKGSIVTVKKVMQKNNILVTRKAYEVKGGLFDQQIMKKGKGQVPIKGND
ERLADIEKYGGYNKAAGTYFMLVKSLDKKGKEIRTIEFVPLYLKNQIEINHESAIQYLAQERGL
NSPEILLSKIKIDTLFKVDGFKMWLSGRTGNQLIFKGANQLILSHQEAAILKGVVKYVNRKNEN
KDAKLSERDGMTEEKLLQLYDTFLDKLSNTVYSIRLSAQIKTLTEKRAKFIGLSNEDQCIVLNE
ILHMFQCQSGSANLKLIGGPGSAGILVMNNNITACKQISVINQSPTGIYEKEIDLIKL
SEQ ID NO: 323
MKKPYSIGLDIGTNSVGWAVVTDDYKVPAKKMKVLGNTDKSHIEKNLLGALLFDSGNTAEDRRL
KRTARRRYTRRRNRILYLQEIFSEEMGKVDDSFFHRLEDSFLVTEDKRGERHPIFGNLEEEVKY
HENFPTIYHLRQYLADNPEKVDLRLVYLALAHIIKFRGHFLIEGKFDTRNNDVQRLFQEFLAVY
DNTFENSSLQEQNVQVEEILTDKISKSAKKDRVLKLFPNEKSNGRFAEFLKLIVGNQADFKKHF
ELEEKAPLQFSKDTYEEELEVLLAQIGDNYAELFLSAKKLYDSILLSGILTVTDVGTKAPLSAS
MIQRYNEHQMDLAQLKQFIRQKLSDKYNEVFSDVSKDGYAGYIDGKTNQEAFYKYLKGLLNKIE
GSGYFLDKIEREDFLRKQRTFDNGSIPHQIHLQEMRAIIRRQAEFYPFLADNQDRIEKLLTFRI
PYYVGPLARGKSDFAWLSRKSADKITPWNFDEIVDKESSAEAFINRMTNYDLYLPNQKVLPKHS
LLYEKFTVYNELTKVKYKTEQGKTAFFDANMKQEIFDGVFKVYRKVTKDKLMDFLEKEFDEFRI
VDLTGLDKENKVFNASYGTYHDLCKILDKDFLDNSKNEKILEDIVLTLTLFEDREMIRKRLENY
SDLLTKEQVKKLERRHYTGWGRLSAELIHGIRNKESRKTILDYLIDDGNSNRNFMQLINDDALS
FKEEIAKAQVIGETDNLNQVVSDIAGSPAIKKGILQSLKIVDELVKIMGHQPENIVVEMARENQ
FTNQGRRNSQQRLKGLTDSIKEFGSQILKEHPVENSQLQNDRLFLYYLQNGRDMYTGEELDIDY
LSQYDIDHIIPQAFIKDNSIDNRVLTSSKENRGKSDDVPSKDVVRKMKSYWSKLLSAKLITQRK
FDNLTKAERGGLTDDDKAGFIKRQLVETRQITKHVARILDERFNTETDENNKKIRQVKIVTLKS
NLVSNFRKEFELYKVREINDYHHAHDAYLNAVIGKALLGVYPQLEPEFVYGDYPHFHGHKENKA
TAKKFFYSNIMNFFKKDDVRTDKNGEIIWKKDEHISNIKKVLSYPQVNIVKKVEEQTGGFSKES
ILPKGNSDKLIPRKTKKFYWDTKKYGGFDSPIVAYSILVIADIEKGKSKKLKTVKALVGVTIME
KMTFERDPVAFLERKGYRNVQEENIIKLPKYSLFKLENGRKRLLASARELQKGNEIVLPNHLGT
LLYHAKNIHKVDEPKHLDYVDKHKDEFKELLDVVSNFSKKYTLAEGNLEKIKELYAQNNGEDLK
ELASSFINLLTFTAIGAPATFKFFDKNIDRKRYTSTTEILNATLIHQSITGLYETRIDLNKLGG
D
SEQ ID NO: 324
MDKKYSIGLDIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD
SEQ ID NO: 325
MTKPYSIGLDIGTNSVGWAVTTDNYKVPSKKMKVLGNTSKKYIKKNLLGVLLFDSGITAEGRRL
KRTARRRYTRRRNRILYLQEIFSTEMATLDDAFFQRLDDSFLVPDDKRDSKYPIFGNLVEEKAY
HDEFPTIYHLRKYLADSTKKADLRLVYLALAHMIKYRGHFLIEGEFNSKNNDIQKNFQDFLDTY
NAIFESDLSLENSKQLEEIVKDKISKLEKKDRILKLFPGEKNSGIFSEFLKLIVGNQADFRKCF
NLDEKASLHFSKESYDEDLETLLGYIGDDYSDVFLKAKKLYDAILLSGFLTVTDNETEAPLSSA
MIKRYNEHKEDLALLKEYIRNISLKTYNEVFKDDTKNGYAGYIDGKTNQEDFYVYLKKLLAEFE
GADYFLEKIDREDFLRKQRTFDNGSIPYQIHLQEMRAILDKQAKFYPFLAKNKERIEKILTFRI
PYYVGPLARGNSDFAWSIRKRNEKITPWNFEDVIDKESSAEAFINRMTSFDLYLPEEKVLPKHS
LLYETFNVYNELTKVRFIAESMRDYQFLDSKQKKDIVRLYFKDKRKVTDKDIIEYLHAIYGYDG
IELKGIEKQFNSSLSTYHDLLNIINDKEFLDDSSNEAIIEEIIHTLTIFEDREMIKQRLSKFEN
IFDKSVLKKLSRRHYTGWGKLSAKLINGIRDEKSGNTILDYLIDDGISNRNFMQLIHDDALSFK
KKIQKAQIIGDEDKGNIKEVVKSLPGSPAIKKGILQSIKIVDELVKVMGGRKPESIVVEMAREN
QYTNQGKSNSQQRLKRLEKSLKELGSKILKENIPAKLSKIDNNALQNDRLYLYYLQNGKDMYTG
DDLDIDRLSNYDIDHIIPQAFLKDNSIDNKVLVSSASNRGKSDDVPSLEVVKKRKTFWYQLLKS
KLISQRKFDNLTKAERGGLSPEDKAGFIQRQLVETRQITKHVARLLDEKFNNKKDENNRAVRTV
KIITLKSTLVSQFRKDFELYKVREINDFHHAHDAYLNAVVASALLKKYPKLEPEFVYGDYPKYN
SFRERKSATEKVYFYSNIMNIFKKSISLADGRVIERPLIEVNEETGESVWNKESDLATVRRVLS
YPQVNVVKKVEEQNHGLDRGKPKGLFNANLSSKPKPNSNENLVGAKEYLDPKKYGGYAGISNSF
TVLVKGTIEKGAKKKITNVLEFQGISILDRINYRKDKLNFLLEKGYKDIELIIELPKYSLFELS
DGSRRMLASILSTNNKRGEIHKGNQIFLSQKFVKLLYHAKRISNTINENHRKYVENHKKEFEEL
FYYILEFNENYVGAKKNGKLLNSAFQSWQNHSIDELCSSFIGPTGSERKGLFELTSRGSAADFE
FLGVKIPRYRDYTPSSLLKDATLIHQSVTGLYETRIDLAKLGEG
SEQ ID NO: 326
MKKQKFSDYYLGFDIGTNSVGWCVTDLDYNVLRFNKKDMWGSRLFDEAKTAAERRVQRNSRRRL
KRRKWRLNLLEEIFSDEIMKIDSNFFRRLKESSLWLEDKNSKEKFTLFNDDNYKDYDFYKQYPT
IFHLRDELIKNPEKKDIRLIYLALHSIFKSRGHFLFEGQNLKEIKNFETLYNNLISFLEDNGIN
KSIDKDNIEKLEKIICDSGKGLKDKEKEFKGIFNSDKQLVAIFKLSVGSSVSLNDLFDTDEYKK
EEVEKEKISFREQIYEDDKPIYYSILGEKIELLDIAKSFYDFMVLNNILSDSNYISEAKVKLYE
EHKKDLKNLKYIIRKYNKENYDKLFKDKNENNYPAYIGLNKEKDKKEVVEKSRLKIDDLIKVIK
GYLPKPERIEEKDKTIFNEILNKIELKTILPKQRISDNGTLPYQIHEVELEKILENQSKYYDFL
NYEENGVSTKDKLLKTFKFRIPYYVGPLNSYHKDKGGNSWIVRKEEGKILPWNFEQKVDIEKSA
EEFIKRMTNKCTYLNGEDVIPKDSFLYSEYIILNELNKVQVNDEFLNEENKRKIIDELFKENKK
VSEKKFKEYLLVNQIANRTVELKGIKDSFNSNYVSYIKFKDIFGEKLNLDIYKEISEKSILWKC
LYGDDKKIFEKKIKNEYGDILNKDEIKKINSFKFNTWGRLSEKLLTGIEFINLETGECYSSVME
ALRRTNYNLMELLSSKFTLQESIDNENKEMNEVSYRDLIEESYVSPSLKRAILQTLKIYEEIKK
ITGRVPKKVFIEMARGGDESMKNKKIPARQEQLKKLYDSCGNDIANFSIDIKEMKNSLSSYDNN
SLRQKKLYLYYLQFGKCMYTGREIDLDRLLQNNDTYDIDHIYPRSKVIKDDSFDNLVLVLKNEN
AEKSNEYPVKKEIQEKMKSFWRFLKEKNFISDEKYKRLTGKDDFELRGFMARQLVNVRQTTKEV
GKILQQIEPEIKIVYSKAEIASSFREMFDFIKVRELNDTHHAKDAYLNIVAGNVYNTKFTEKPY
RYLQEIKENYDVKKIYNYDIKNAWDKENSLEIVKKNMEKNTVNITRFIKEEKGELFNLNPIKKG
ETSNEIISIKPKLYDGKDNKLNEKYGYYTSLKAAYFIYVEHEKKNKKVKTFERITRIDSTLIKN
EKNLIKYLVSQKKLLNPKIIKKIYKEQTLIIDSYPYTFTGVDSNKKVELKNKKQLYLEKKYEQI
LKNALKFVEDNQGETEENYKFIYLKKRNNNEKNETIDAVKERYNIEFNEMYDKFLEKLSSKDYK
NYINNKLYTNFLNSKEKFKKLKLWEKSLILREFLKIFNKNTYGKYEIKDSQTKEKLFSFPEDTG
RIRLGQSSLGNNKELLEESVTGLFVKKIKL
SEQ ID NO: 327
MKNYTIGLDIGVASVGWVCIDENYKILNYNNRHAFGVHEFESAESAAGRRLKRGMRRRYNRRKK
RLQLLQSLFDSYITDSGFFSKTDSQHFWKNNNEFENRSLTEVLSSLRISSRKYPTIYHLRSDLI
ESNKKMDLRLVYLALHNLVKYRGHFLQEGNWSEAASAEGMDDQLLELVTRYAELENLSPLDLSE
SQWKAAETLLLNRNLTKTDQSKELTAMFGKEYEPFCKLVAGLGVSLHQLFPSSEQALAYKETKT
KVQLSNENVEEVMELLLEEESALLEAVQPFYQQVVLYELLKGETYVAKAKVSAFKQYQKDMASL
KNLLDKTFGEKVYRSYFISDKNSQREYQKSHKVEVLCKLDQFNKEAKFAETFYKDLKKLLEDKS
KTSIGTTEKDEMLRIIKAIDSNQFLQKQKGIQNAAIPHQNSLYEAEKILRNQQAHYPFITTEWI
EKVKQILAFRIPYYIGPLVKDTTQSPFSWVERKGDAPITPWNFDEQIDKAASAEAFISRMRKTC
TYLKGQEVLPKSSLTYERFEVLNELNGIQLRTTGAESDFRHRLSYEMKCWIIDNVFKQYKTVST
KRLLQELKKSPYADELYDEHTGEIKEVFGTQKENAFATSLSGYISMKSILGAVVDDNPAMTEEL
IYWIAVFEDREILHLKIQEKYPSITDVQRQKLALVKLPGWGRFSRLLIDGLPLDEQGQSVLDHM
EQYSSVFMEVLKNKGFGLEKKIQKMNQHQVDGTKKIRYEDIEELAGSPALKRGIWRSVKIVEEL
VSIFGEPANIVLEVAREDGEKKRTKSRKDQWEELTKTTLKNDPDLKSFIGEIKSQGDQRFNEQR
FWLYVTQQGKCLYTGKALDIQNLSMYEVDHILPQNFVKDDSLDNLALVMPEANQRKNQVGQNKM
PLEIIEANQQYAMRTLWERLHELKLISSGKLGRLKKPSFDEVDKDKFIARQLVETRQIIKHVRD
LLDERFSKSDIHLVKAGIVSKFRRFSEIPKIRDYNNKHHAMDALFAAALIQSILGKYGKNFLAF
DLSKKDRQKQWRSVKGSNKEFFLFKNFGNLRLQSPVTGEEVSGVEYMKHVYFELPWQTTKMTQT
GDGMFYKESIFSPKVKQAKYVSPKTEKFVHDEVKNHSICLVEFTFMKKEKEVQETKFIDLKVIE
HHQFLKEPESQLAKFLAEKETNSPIIHARIIRTIPKYQKIWIEHFPYYFISTRELHNARQFEIS
YELMEKVKQLSERSSVEELKIVFGLLIDQMNDNYPIYTKSSIQDRVQKFVDTQLYDFKSFEIGF
EELKKAVAANAQRSDTFGSRISKKPKPEEVAIGYESITGLKYRKPRSVVGTKR
SEQ ID NO: 328
MKKEIKDYFLGLDVGTGSVGWAVTDTDYKLLKANRKDLWGMRCFETAETAEVRRLHRGARRRIE
RRKKRIKLLQELFSQEIAKTDEGFFQRMKESPFYAEDKTILQENTLFNDKDFADKTYHKAYPTI
NHLIKAWIENKVKPDPRLLYLACHNIIKKRGHFLFEGDFDSENQFDTSIQALFEYLREDMEVDI
DADSQKVKEILKDSSLKNSEKQSRLNKILGLKPSDKQKKAITNLISGNKINFADLYDNPDLKDA
EKNSISFSKDDFDALSDDLASILGDSFELLLKAKAVYNCSVLSKVIGDEQYLSFAKVKIYEKHK
TDLTKLKNVIKKHFPKDYKKVFGYNKNEKNNNNYSGYVGVCKTKSKKLIINNSVNQEDFYKFLK
TILSAKSEIKEVNDILTEIETGTFLPKQISKSNAEIPYQLRKMELEKILSNAEKHFSFLKQKDE
KGLSHSEKIIMLLTFKIPYYIGPINDNHKKFFPDRCWVVKKEKSPSGKTTPWNFFDHIDKEKTA
EAFITSRTNFCTYLVGESVLPKSSLLYSEYTVLNEINNLQIIIDGKNICDIKLKQKIYEDLFKK
YKKITQKQISTFIKHEGICNKTDEVIILGIDKECTSSLKSYIELKNIFGKQVDEISTKNMLEEI
IRWATIYDEGEGKTILKTKIKAEYGKYCSDEQIKKILNLKFSGWGRLSRKFLETVTSEMPGFSE
PVNIITAMRETQNNLMELLSSEFTFTENIKKINSGFEDAEKQFSYDGLVKPLFLSPSVKKMLWQ
TLKLVKEISHITQAPPKKIFIEMAKGAELEPARTKTRLKILQDLYNNCKNDADAFSSEIKDLSG
KIENEDNLRLRSDKLYLYYTQLGKCMYCGKPIEIGHVFDTSNYDIDHIYPQSKIKDDSISNRVL
VCSSCNKNKEDKYPLKSEIQSKQRGFWNFLQRNNFISLEKLNRLTRATPISDDETAKFIARQLV
ETRQATKVAAKVLEKMFPETKIVYSKAETVSMFRNKFDIVKCREINDFHHAHDAYLNIVVGNVY
NTKFTNNPWNFIKEKRDNPKIADTYNYYKVFDYDVKRNNITAWEKGKTIITVKDMLKRNTPIYT
RQAACKKGELFNQTIMKKGLGQHPLKKEGPFSNISKYGGYNKVSAAYYTLIEYEEKGNKIRSLE
TIPLYLVKDIQKDQDVLKSYLTDLLGKKEFKILVPKIKINSLLKINGFPCHITGKTNDSFLLRP
AVQFCCSNNEVLYFKKIIRFSEIRSQREKIGKTISPYEDLSFRSYIKENLWKKTKNDEIGEKEF
YDLLQKKNLEIYDMLLTKHKDTIYKKRPNSATIDILVKGKEKFKSLIIENQFEVILEILKLFSA
TRNVSDLQHIGGSKYSGVAKIGNKISSLDNCILIYQSITGIFEKRIDLLKV
SEQ ID NO: 329
MEGQMKNNGNNLQQGNYYLGLDVGTSSVGWAVTDTDYNVLKFRGKSMWGARLFDEASTAEERRT
HRGNRRRLARRKYRLLLLEQLFEKEIRKIDDNFFVRLHESNLWADDKSKPSKFLLFNDTNFTDK
DYLKKYPTIYHLRSDLIHNSTEHDIRLVFLALHHLIKYRGHFIYDNSANGDVKTLDEAVSDFEE
YLNENDIEFNIENKKEFINVLSDKHLTKKEKKISLKKLYGDITDSENINISVLIEMLSGSSISL
SNLFKDIEFDGKQNLSLDSDIEETLNDVVDILGDNIDLLIHAKEVYDIAVLTSSLGKHKYLCDA
KVELFEKNKKDLMILKKYIKKNHPEDYKKIFSSPTEKKNYAAYSQTNSKNVCSQEEFCLFIKPY
IRDMVKSENEDEVRIAKEVEDKSFLTKLKGTNNSVVPYQIHERELNQILKNIVAYLPFMNDEQE
DISVVDKIKLIFKFKIPYYVGPLNTKSTRSWVYRSDEKIYPWNFSNVIDLDKTAHEFMNRLIGR
CTYTNDPVLPMDSLLYSKYNVLNEINPIKVNGKAIPVEVKQAIYTDLFENSKKKVTRKSIYIYL
LKNGYIEKEDIVSGIDIEIKSKLKSHHDFTQIVQENKCTPEEIERIIKGILVYSDDKSMLRRWL
KNNIKGLSENDVKYLAKLNYKEWGRLSKTLLTDIYTINPEDGEACSILDIMWNTNATLMEILSN
EKYQFKQNIENYKAENYDEKQNLHEELDDMYISPAARRSIWQALRIVDEIVDIKKSAPKKIFIE
MAREKKSAMKKKRTESRKDTLLELYKSCKSQADGFYDEELFEKLSNESNSRLRRDQLYLYYTQM
GRSMYTGKRIDFDKLINDKNTYDIDHIYPRSKIKDDSITNRVLVEKDINGEKTDIYPISEDIRQ
KMQPFWKILKEKGLINEEKYKRLTRNYELTDEELSSFVARQLVETQQSTKALATLLKKEYPSAK
IVYSKAGNVSEFRNRKDKELPKFREINDLHHAKDAYLNIVVGNVYDTKFTEKFFNNIRNENYSL
KRVFDFSVPGAWDAKGSTFNTIKKYMAKNNPIIAFAPYEVKGELFDQQIVPKGKGQFPIKQGKD
IEKYGGYNKLSSAFLFAVEYKGKKARERSLETVYIKDVELYLQDPIKYCESVLGLKEPQIIKPK
ILMGSLFSINNKKLVVTGRSGKQYVCHHIYQLSINDEDSQYLKNIAKYLQEEPDGNIERQNILN
ITSVNNIKLFDVLCTKFNSNTYEIILNSLKNDVNEGREKFSELDILEQCNILLQLLKAFKCNRE
SSNLEKLNNKKQAGVIVIPHLFTKCSVFKVIHQSITGLFEKEMDLLK
SEQ ID NO: 330
MGRKPYILSLDIGTGSVGYACMDKGFNVLKYHDKDALGVYLFDGALTAQERRQFRTSRRRKNRR
IKRLGLLQELLAPLVQNPNFYQFQRQFAWKNDNMDFKNKSLSEVLSFLGYESKKYPTIYHLQEA
LLLKDEKFDPELIYMALYHLVKYRGHFLFDHLKIENLTNNDNMHDFVELIETYENLNNIKLNLD
YEKTKVIYEILKDNEMTKNDRAKRVKNMEKKLEQFSIMLLGLKFNEGKLFNHADNAEELKGANQ
SHTFADNYEENLTPFLTVEQSEFIERANKIYLSLTLQDILKGKKSMAMSKVAAYDKFRNELKQV
KDIVYKADSTRTQFKKIFVSSKKSLKQYDATPNDQTFSSLCLFDQYLIRPKKQYSLLIKELKKI
IPQDSELYFEAENDTLLKVLNTTDNASIPMQINLYEAETILRNQQKYHAEITDEMIEKVLSLIQ
FRIPYYVGPLVNDHTASKFGWMERKSNESIKPWNFDEVVDRSKSATQFIRRMTNKCSYLINEDV
LPKNSLLYQEMEVLNELNATQIRLQTDPKNRKYRMMPQIKLFAVEHIFKKYKTVSHSKFLEIML
NSNHRENFMNHGEKLSIFGTQDDKKFASKLSSYQDMTKIFGDIEGKRAQIEEIIQWITIFEDKK
ILVQKLKECYPELTSKQINQLKKLNYSGWGRLSEKLLTHAYQGHSIIELLRHSDENFMEILTND
VYGFQNFIKEENQVQSNKIQHQDIANLTTSPALKKGIWSTIKLVRELTSIFGEPEKIIMEFATE
DQQKGKKQKSRKQLWDDNIKKNKLKSVDEYKYIIDVANKLNNEQLQQEKLWLYLSQNGKCMYSG
QSIDLDALLSPNATKHYEVDHIFPRSFIKDDSIDNKVLVIKKMNQTKGDQVPLQFIQQPYERIA
YWKSLNKAGLISDSKLHKLMKPEFTAMDKEGFIQRQLVETRQISVHVRDFLKEEYPNTKVIPMK
AKMVSEFRKKFDIPKIRQMNDAHHAIDAYLNGVVYHGAQLAYPNVDLFDFNFKWEKVREKWKAL
GEFNTKQKSRELFFFKKLEKMEVSQGERLISKIKLDMNHFKINYSRKLANIPQQFYNQTAVSPK
TAELKYESNKSNEVVYKGLTPYQTYVVAIKSVNKKGKEKMEYQMIDHYVFDFYKFQNGNEKELA
LYLAQRENKDEVLDAQIVYSLNKGDLLYINNHPCYFVSRKEVINAKQFELTVEQQLSLYNVMNN
KETNVEKLLIEYDFIAEKVINEYHHYLNSKLKEKRVRTFFSESNQTHEDFIKALDELFKVVTAS
ATRSDKIGSRKNSMTHRAFLGKGKDVKIAYTSISGLKTTKPKSLFKLAESRNEL
SEQ ID NO: 331
MAKILGLDLGTNSIGWAVVERENIDFSLIDKGVRIFSEGVKSEKGIESSRAAERTGYRSARKIK
YRRKLRKYETLKVLSLNRMCPLSIEEVEEWKKSGFKDYPLNPEFLKWLSTDEESNVNPYFFRDR
ASKHKVSLFELGRAFYHIAQRRGFLSNRLDQSAEGILEEHCPKIEAIVEDLISIDEISTNITDY
FFETGILDSNEKNGYAKDLDEGDKKLVSLYKSLLAILKKNESDFENCKSEIIERLNKKDVLGKV
KGKIKDISQAMLDGNYKTLGQYFYSLYSKEKIRNQYTSREEHYLSEFITICKVQGIDQINEEEK
INEKKFDGLAKDLYKAIFFQRPLKSQKGLIGKCSFEKSKSRCAISHPDFEEYRMWTYLNTIKIG
TQSDKKLRFLTQDEKLKLVPKFYRKNDFNFDVLAKELIEKGSSFGFYKSSKKNDFFYWFNYKPT
DTVAACQVAASLKNAIGEDWKTKSFKYQTINSNKEQVSRTVDYKDLWHLLTVATSDVYLYEFAI
DKLGLDEKNAKAFSKTKLKKDFASLSLSAINKILPYLKEGLLYSHAVFVANIENIVDENIWKDE
KQRDYIKTQISEIIENYTLEKSRFEIINGLLKEYKSENEDGKRVYYSKEAEQSFENDLKKKLVL
FYKSNEIENKEQQETIFNELLPIFIQQLKDYEFIKIQRLDQKVLIFLKGKNETGQIFCTEEKGT
AEEKEKKIKNRLKKLYHPSDIEKFKKKIIKDEFGNEKIVLGSPLTPSIKNPMAMRALHQLRKVL
NALILEGQIDEKTIIHIEMARELNDANKRKGIQDYQNDNKKFREDAIKEIKKLYFEDCKKEVEP
TEDDILRYQLWMEQNRSEIYEEGKNISICDIIGSNPAYDIEHTIPRSRSQDNSQMNKTLCSQRF
NREVKKQSMPIELNNHLEILPRIAHWKEEADNLTREIEIISRSIKAAATKEIKDKKIRRRHYLT
LKRDYLQGKYDRFIWEEPKVGFKNSQIPDTGIITKYAQAYLKSYFKKVESVKGGMVAEFRKIWG
IQESFIDENGMKHYKVKDRSKHTHHTIDAITIACMTKEKYDVLAHAWTLEDQQNKKEARSIIEA
SKPWKTFKEDLLKIEEEILVSHYTPDNVKKQAKKIVRVRGKKQFVAEVERDVNGKAVPKKAASG
KTIYKLDGEGKKLPRLQQGDTIRGSLHQDSIYGAIKNPLNTDEIKYVIRKDLESIKGSDVESIV
DEVVKEKIKEAIANKVLLLSSNAQQKNKLVGTVWMNEEKRIAINKVRIYANSVKNPLHIKEHSL
LSKSKHVHKQKVYGQNDENYAMAIYELDGKRDFELINIFNLAKLIKQGQGFYPLHKKKEIKGKI
VFVPIEKRNKRDVVLKRGQQVVFYDKEVENPKDISEIVDFKGRIYIIEGLSIQRIVRPSGKVDE
YGVIMLRYFKEARKADDIKQDNFKPDGVFKLGENKPTRKMNHQFTAFVEGIDFKVLPSGKFEKI
SEQ ID NO: 332
MEFKKVLGLDIGTNSIGCALLSLPKSIQDYGKGGRLEWLTSRVIPLDADYMKAFIDGKNGLPQV
ITPAGKRRQKRGSRRLKHRYKLRRSRLIRVFKTLNWLPEDFPLDNPKRIKETISTEGKFSFRIS
DYVPISDESYREFYREFGYPENEIEQVIEEINFRRKTKGKNKNPMIKLLPEDWVVYYLRKKALI
KPTTKEELIRIIYLFNQRRGFKSSRKDLTETAILDYDEFAKRLAEKEKYSAENYETKFVSITKV
KEVVELKTDGRKGKKRFKVILEDSRIEPYEIERKEKPDWEGKEYTFLVTQKLEKGKFKQNKPDL
PKEEDWALCTTALDNRMGSKHPGEFFFDELLKAFKEKRGYKIRQYPVNRWRYKKELEFIWTKQC
QLNPELNNLNINKEILRKLATVLYPSQSKFFGPKIKEFENSDVLHIISEDIIYYQRDLKSQKSL
ISECRYEKRKGIDGEIYGLKCIPKSSPLYQEFRIWQDIHNIKVIRKESEVNGKKKINIDETQLY
INENIKEKLFELFNSKDSLSEKDILELISLNIINSGIKISKKEEETTHRINLFANRKELKGNET
KSRYRKVFKKLGFDGEYILNHPSKLNRLWHSDYSNDYADKEKTEKSILSSLGWKNRNGKWEKSK
NYDVFNLPLEVAKAIANLPPLKKEYGSYSALAIRKMLVVMRDGKYWQHPDQIAKDQENTSLMLF
DKNLIQLTNNQRKVLNKYLLTLAEVQKRSTLIKQKLNEIEHNPYKLELVSDQDLEKQVLKSFLE
KKNESDYLKGLKTYQAGYLIYGKHSEKDVPIVNSPDELGEYIRKKLPNNSLRNPIVEQVIRETI
FIVRDVWKSFGIIDEIHIELGRELKNNSEERKKTSESQEKNFQEKERARKLLKELLNSSNFEHY
DENGNKIFSSFTVNPNPDSPLDIEKFRIWKNQSGLTDEELNKKLKDEKIPTEIEVKKYILWLTQ
KCRSPYTGKIIPLSKLFDSNVYEIEHIIPRSKMKNDSTNNLVICELGVNKAKGDRLAANFISES
NGKCKFGEVEYTLLKYGDYLQYCKDTFKYQKAKYKNLLATEPPEDFIERQINDTRYIGRKLAEL
LTPVVKDSKNIIFTIGSITSELKITWGLNGVWKDILRPRFKRLESIINKKLIFQDEDDPNKYHF
DLSINPQLDKEGLKRLDHRHHALDATIIAATTREHVRYLNSLNAADNDEEKREYFLSLCNHKIR
DFKLPWENFTSEVKSKLLSCVVSYKESKPILSDPFNKYLKWEYKNGKWQKVFAIQIKNDRWKAV
RRSMFKEPIGTVWIKKIKEVSLKEAIKIQAIWEEVKNDPVRKKKEKYIYDDYAQKVIAKIVQEL
GLSSSMRKQDDEKLNKFINEAKVSAGVNKNLNTTNKTIYNLEGRFYEKIKVAEYVLYKAKRMPL
NKKEYIEKLSLQKMFNDLPNFILEKSILDNYPEILKELESDNKYIIEPHKKNNPVNRLLLEHIL
EYHNNPKEAFSTEGLEKLNKKAINKIGKPIKYITRLDGDINEEEIFRGAVFETDKGSNVYFVMY
ENNQTKDREFLKPNPSISVLKAIEHKNKIDFFAPNRLGFSRIILSPGDLVYVPTNDQYVLIKDN
SSNETIINWDDNEFISNRIYQVKKFTGNSCYFLKNDIASLILSYSASNGVGEFGSQNISEYSVD
DPPIRIKDVCIKIRVDRLGNVRPL
SEQ ID NO: 333
MKHILGLDLGTNSIGWALIERNIEEKYGKIIGMGSRIVPMGAELSKFEQGQAQTKNADRRTNRG
ARRLNKRYKQRRNKLIYILQKLDMLPSQIKLKEDFSDPNKIDKITILPISKKQEQLTAFDLVSL
RVKALTEKVGLEDLGKIIYKYNQLRGYAGGSLEPEKEDIFDEEQSKDKKNKSFIAFSKIVFLGE
PQEEIFKNKKLNRRAIIVETEEGNFEGSTFLENIKVGDSLELLINISASKSGDTITIKLPNKTN
WRKKMENIENQLKEKSKEMGREFYISEFLLELLKENRWAKIRNNTILRARYESEFEAIWNEQVK
HYPFLENLDKKTLIEIVSFIFPGEKESQKKYRELGLEKGLKYIIKNQVVFYQRELKDQSHLISD
CRYEPNEKAIAKSHPVFQEYKVWEQINKLIVNTKIEAGTNRKGEKKYKYIDRPIPTALKEWIFE
ELQNKKEITFSAIFKKLKAEFDLREGIDFLNGMSPKDKLKGNETKLQLQKSLGELWDVLGLDSI
NRQIELWNILYNEKGNEYDLTSDRTSKVLEFINKYGNNIVDDNAEETAIRISKIKFARAYSSLS
LKAVERILPLVRAGKYFNNDFSQQLQSKILKLLNENVEDPFAKAAQTYLDNNQSVLSEGGVGNS
IATILVYDKHTAKEYSHDELYKSYKEINLLKQGDLRNPLVEQIINEALVLIRDIWKNYGIKPNE
IRVELARDLKNSAKERATIHKRNKDNQTINNKIKETLVKNKKELSLANIEKVKLWEAQRHLSPY
TGQPIPLSDLFDKEKYDVDHIIPISRYFDDSFTNKVISEKSVNQEKANRTAMEYFEVGSLKYSI
FTKEQFIAHVNEYFSGVKRKNLLATSIPEDPVQRQIKDTQYIAIRVKEELNKIVGNENVKTTTG
SITDYLRNHWGLTDKFKLLLKERYEALLESEKFLEAEYDNYKKDFDSRKKEYEEKEVLFEEQEL
TREEFIKEYKENYIRYKKNKLIIKGWSKRIDHRHHAIDALIVACTEPAHIKRLNDLNKVLQDWL
VEHKSEFMPNFEGSNSELLEEILSLPENERTEIFTQIEKFRAIEMPWKGFPEQVEQKLKEIIIS
HKPKDKLLLQYNKAGDRQIKLRGQLHEGTLYGISQGKEAYRIPLTKFGGSKFATEKNIQKIVSP
FLSGFIANHLKEYNNKKEEAFSAEGIMDLNNKLAQYRNEKGELKPHTPISTVKIYYKDPSKNKK
KKDEEDLSLQKLDREKAFNEKLYVKTGDNYLFAVLEGEIKTKKTSQIKRLYDIISFFDATNFLK
EEFRNAPDKKTFDKDLLFRQYFEERNKAKLLFTLKQGDFVYLPNENEEVILDKESPLYNQYWGD
LKERGKNIYVVQKFSKKQIYFIKHTIADIIKKDVEFGSQNCYETVEGRSIKENCFKLEIDRLGN
IVKVIKR
SEQ ID NO: 334
MHVEIDFPHFSRGDSHLAMNKNEILRGSSVLYRLGLDLGSNSLGWFVTHLEKRGDRHEPVALGP
GGVRIFPDGRDPQSGTSNAVDRRMARGARKRRDRFVERRKELIAALIKYNLLPDDARERRALEV
LDPYALRKTALTDTLPAHHVGRALFHLNQRRGFQSNRKTDSKQSEDGAIKQAASRLATDKGNET
LGVFFADMHLRKSYEDRQTAIRAELVRLGKDHLTGNARKKIWAKVRKRLFGDEVLPRADAPHGV
RARATITGTKASYDYYPTRDMLRDEFNAIWAGQSAHHATITDEARTEIEHIIFYQRPLKPAIVG
KCTLDPATRPFKEDPEGYRAPWSHPLAQRFRILSEARNLEIRDTGKGSRRLTKEQSDLVVAALL
ANREVKFDKLRTLLKLPAEARFNLESDRRAALDGDQTAARLSDKKGFNKAWRGFPPERQIAIVA
RLEETEDENELIAWLEKECALDGAAAARVANTTLPDGHCRLGLRAIKKIVPIMQDGLDEDGVAG
AGYHIAAKRAGYDHAKLPTGEQLGRLPYYGQWLQDAVVGSGDARDQKEKQYGQFPNPTVHIGLG
QLRRVVNDLIDKYGPPTEISIEFTRALKLSEQQKAERQREQRRNQDKNKARAEELAKFGRPANP
RNLLKMRLWEELAHDPLDRKCVYTGEQISIERLLSDEVDIDHILPVAMTLDDSPANKIICMRYA
NRHKRKQTPSEAFGSSPTLQGHRYNWDDIAARATGLPRNKRWRFDANAREEFDKRGGFLARQLN
ETGWLARLAKQYLGAVTDPNQIWVVPGRLTSMLRGKWGLNGLLPSDNYAGVQDKAEEFLASTDD
MEFSGVKNRADHRHHAIDGLVTALTDRSLLWKMANAYDEEHEKFVIEPPWPTMRDDLKAALEKM
VVSHKPDHGIEGKLHEDSAYGFVKPLDATGLKEEEAGNLVYRKAIESLNENEVDRIRDIQLRTI
VRDHVNVEKTKGVALADALRQLQAPSDDYPQFKHGLRHVRILKKEKGDYLVPIANRASGVAYKA
YSAGENFCVEVFETAGGKWDGEAVRRFDANKKNAGPKIAHAPQWRDANEGAKLVMRIHKGDLIR
LDHEGRARIMVVHRLDAAAGRFKLADHNETGNLDKRHATNNDIDPFRWLMASYNTLKKLAAVPV
RVDELGRVWRVMPN
SEQ ID NO: 335
METTLGIDLGTNSIGLALVDQEEHQILYSGVRIFPEGINKDTIGLGEKEESRNATRRAKRQMRR
QYFRKKLRKAKLLELLIAYDMCPLKPEDVRRWKNWDKQQKSTVRQFPDTPAFREWLKQNPYELR
KQAVTEDVTRPELGRILYQMIQRRGFLSSRKGKEEGKIFTGKDRMVGIDETRKNLQKQTLGAYL
YDIAPKNGEKYRFRTERVRARYTLRDMYIREFEIIWQRQAGHLGLAHEQATRKKNIFLEGSATN
VRNSKLITHLQAKYGRGHVLIEDTRITVTFQLPLKEVLGGKIEIEEEQLKFKSNESVLFWQRPL
RSQKSLLSKCVFEGRNFYDPVHQKWIIAGPTPAPLSHPEFEEFRAYQFINNIIYGKNEHLTAIQ
REAVFELMCTESKDFNFEKIPKHLKLFEKFNFDDTTKVPACTTISQLRKLFPHPVWEEKREEIW
HCFYFYDDNTLLFEKLQKDYALQTNDLEKIKKIRLSESYGNVSLKAIRRINPYLKKGYAYSTAV
LLGGIRNSFGKRFEYFKEYEPEIEKAVCRILKEKNAEGEVIRKIKDYLVHNRFGFAKNDRAFQK
LYHHSQAITTQAQKERLPETGNLRNPIVQQGLNELRRTVNKLLATCREKYGPSFKFDHIHVEMG
RELRSSKTEREKQSRQIRENEKKNEAAKVKLAEYGLKAYRDNIQKYLLYKEIEEKGGTVCCPYT
GKTLNISHTLGSDNSVQIEHIIPYSISLDDSLANKTLCDATFNREKGELTPYDFYQKDPSPEKW
GASSWEEIEDRAFRLLPYAKAQRFIRRKPQESNEFISRQLNDTRYISKKAVEYLSAICSDVKAF
PGQLTAELRHLWGLNNILQSAPDITFPLPVSATENHREYYVITNEQNEVIRLFPKQGETPRTEK
GELLLTGEVERKVFRCKGMQEFQTDVSDGKYWRRIKLSSSVTWSPLFAPKPISADGQIVLKGRI
EKGVFVCNQLKQKLKTGLPDGSYWISLPVISQTFKEGESVNNSKLTSQQVQLFGRVREGIFRCH
NYQCPASGADGNFWCTLDTDTAQPAFTPIKNAPPGVGGGQIILTGDVDDKGIFHADDDLHYELP
ASLPKGKYYGIFTVESCDPTLIPIELSAPKTSKGENLIEGNIWVDEHTGEVRFDPKKNREDQRH
HAIDAIVIALSSQSLFQRLSTYNARRENKKRGLDSTEHFPSPWPGFAQDVRQSVVPLLVSYKQN
PKTLCKISKTLYKDGKKIHSCGNAVRGQLHKETVYGQRTAPGATEKSYHIRKDIRELKTSKHIG
KVVDITIRQMLLKHLQENYHIDITQEFNIPSNAFFKEGVYRIFLPNKHGEPVPIKKIRMKEELG
NAERLKDNINQYVNPRNNHHVMIYQDADGNLKEEIVSFWSVIERQNQGQPIYQLPREGRNIVSI
LQINDTFLIGLKEEEPEVYRNDLSTLSKHLYRVQKLSGMYYTFRHHLASTLNNEREEFRIQSLE
AWKRANPVKVQIDEIGRITFLNGPLC
SEQ ID NO: 336
MESSQILSPIGIDLGGKFTGVCLSHLEAFAELPNHANTKYSVILIDHNNFQLSQAQRRATRHRV
RNKKRNQFVKRVALQLFQHILSRDLNAKEETALCHYLNNRGYTYVDTDLDEYIKDETTINLLKE
LLPSESEHNFIDWFLQKMQSSEFRKILVSKVEEKKDDKELKNAVKNIKNFITGFEKNSVEGHRH
RKVYFENIKSDITKDNQLDSIKKKIPSVCLSNLLGHLSNLQWKNLHRYLAKNPKQFDEQTFGNE
FLRMLKNFRHLKGSQESLAVRNLIQQLEQSQDYISILEKTPPEITIPPYEARTNTGMEKDQSLL
LNPEKLNNLYPNWRNLIPGIIDAHPFLEKDLEHTKLRDRKRIISPSKQDEKRDSYILQRYLDLN
KKIDKFKIKKQLSFLGQGKQLPANLIETQKEMETHFNSSLVSVLIQIASAYNKEREDAAQGIWF
DNAFSLCELSNINPPRKQKILPLLVGAILSEDFINNKDKWAKFKIFWNTHKIGRTSLKSKCKEI
EEARKNSGNAFKIDYEEALNHPEHSNNKALIKIIQTIPDIIQAIQSHLGHNDSQALIYHNPFSL
SQLYTILETKRDGFHKNCVAVTCENYWRSQKTEIDPEISYASRLPADSVRPFDGVLARMMQRLA
YEIAMAKWEQIKHIPDNSSLLIPIYLEQNRFEFEESFKKIKGSSSDKTLEQAIEKQNIQWEEKF
QRIINASMNICPYKGASIGGQGEIDHIYPRSLSKKHFGVIFNSEVNLIYCSSQGNREKKEEHYL
LEHLSPLYLKHQFGTDNVSDIKNFISQNVANIKKYISFHLLTPEQQKAARHALFLDYDDEAFKT
ITKFLMSQQKARVNGTQKFLGKQIMEFLSTLADSKQLQLEFSIKQITAEEVHDHRELLSKQEPK
LVKSRQQSFPSHAIDATLTMSIGLKEFPQFSQELDNSWFINHLMPDEVHLNPVRSKEKYNKPNI
SSTPLFKDSLYAERFIPVWVKGETFAIGFSEKDLFEIKPSNKEKLFTLLKTYSTKNPGESLQEL
QAKSKAKWLYFPINKTLALEFLHHYFHKEIVTPDDTTVCHFINSLRYYTKKESITVKILKEPMP
VLSVKFESSKKNVLGSFKHTIALPATKDWERLFNHPNFLALKANPAPNPKEFNEFIRKYFLSDN
NPNSDIPNNGHNIKPQKHKAVRKVFSLPVIPGNAGTMMRIRRKDNKGQPLYQLQTIDDTPSMGI
QINEDRLVKQEVLMDAYKTRNLSTIDGINNSEGQAYATFDNWLTLPVSTFKPEIIKLEMKPHSK
TRRYIRITQSLADFIKTIDEALMIKPSDSIDDPLNMPNEIVCKNKLFGNELKPRDGKMKIVSTG
KIVTYEFESDSTPQWIQTLYVTQLKKQP
SEQ ID NO: 337
MKKIVGLDLGTNSIGWALINAYINKEHLYGIEACGSRIIPMDAAILGNFDKGNSISQTADRTSY
RGIRRLRERHLLRRERLHRILDLLGFLPKHYSDSLNRYGKFLNDIECKLPWVKDETGSYKFIFQ
ESFKEMLANFTEHHPILIANNKKVPYDWTIYYLRKKALTQKISKEELAWILLNFNQKRGYYQLR
GEEEETPNKLVEYYSLKVEKVEDSGERKGKDTWYNVHLENGMIYRRTSNIPLDWEGKTKEFIVT
TDLEADGSPKKDKEGNIKRSFRAPKDDDWTLIKKKTEADIDKIKMTVGAYIYDTLLQKPDQKIR
GKLVRTIERKYYKNELYQILKTQSEFHEELRDKQLYIACLNELYPNNEPRRNSISTRDFCHLFI
EDIIFYQRPLKSKKSLIDNCPYEENRYIDKESGEIKHASIKCIAKSHPLYQEFRLWQFIVNLRI
YRKETDVDVTQELLPTEADYVTLFEWLNEKKEIDQKAFFKYPPFGFKKTTSNYRWNYVEDKPYP
CNETHAQIIARLGKAHIPKAFLSKEKEETLWHILYSIEDKQEIEKALHSFANKNNLSEEFIEQF
KNFPPFKKEYGSYSAKAIKKLLPLMRMGKYWSIENIDNGTRIRINKIIDGEYDENIRERVRQKA
INLTDITHFRALPLWLACYLVYDRHSEVKDIVKWKTPKDIDLYLKSFKQHSLRNPIVEQVITET
LRTVRDIWQQVGHIDEIHIELGREMKNPADKRARMSQQMIKNENTNLRIKALLTEFLNPEFGIE
NVRPYSPSQQDLLRIYEEGVLNSILELPEDIGIILGKFNQTDTLKRPTRSEILRYKLWLEQKYR
SPYTGEMIPLSKLFTPAYEIEHIIPQSRYFDDSLSNKVICESEINKLKDRSLGYEFIKNHHGEK
VELAFDKPVEVLSVEAYEKLVHESYSHNRSKMKKLLMEDIPDQFIERQLNDSRYISKVVKSLLS
NIVREENEQEAISKNVIPCTGGITDRLKKDWGINDVWNKIVLPRFIRLNELTESTRFTSINTNN
TMIPSMPLELQKGFNKKRIDHRHHAMDAIIIACANRNIVNYLNNVSASKNTKITRRDLQTLLCH
KDKTDNNGNYKWVIDKPWETFTQDTLTALQKITVSFKQNLRVINKTTNHYQHYENGKKIVSNQS
KGDSWAIRKSMHKETVHGEVNLRMIKTVSFNEALKKPQAIVEMDLKKKILAMLELGYDTKRIKN
YFEENKDTWQDINPSKIKVYYFTKETKDRYFAVRKPIDTSFDKKKIKESITDTGIQQIMLRHLE
TKDNDPTLAFSPDGIDEMNRNILILNKGKKHQPIYKVRVYEKAEKFTVGQKGNKRTKFVEAAKG
TNLFFAIYETEEIDKDTKKVIRKRSYSTIPLNVVIERQKQGLSSAPEDENGNLPKYILSPNDLV
YVPTQEEINKGEVVMPIDRDRIYKMVDSSGITANFIPASTANLIFALPKATAEIYCNGENCIQN
EYGIGSPQSKNQKAITGEMVKEICFPIKVDRLGNIIQVGSCILTN
SEQ ID NO: 338
MSRSLTFSFDIGYASIGWAVIASASHDDADPSVCGCGTVLFPKDDCQAFKRREYRRLRRNIRSR
RVRIERIGRLLVQAQIITPEMKETSGHPAPFYLASEALKGHRTLAPIELWHVLRWYAHNRGYDN
NASWSNSLSEDGGNGEDTERVKHAQDLMDKHGTATMAETICRELKLEEGKADAPMEVSTPAYKN
LNTAFPRLIVEKEVRRILELSAPLIPGLTAEIIELIAQHHPLTTEQRGVLLQHGIKLARRYRGS
LLFGQLIPRFDNRIISRCPVTWAQVYEAELKKGNSEQSARERAEKLSKVPTANCPEFYEYRMAR
ILCNIRADGEPLSAEIRRELMNQARQEGKLTKASLEKAISSRLGKETETNVSNYFTLHPDSEEA
LYLNPAVEVLQRSGIGQILSPSVYRIAANRLRRGKSVTPNYLLNLLKSRGESGEALEKKIEKES
KKKEADYADTPLKPKYATGRAPYARTVLKKVVEEILDGEDPTRPARGEAHPDGELKAHDGCLYC
LLDTDSSVNQHQKERRLDTMTNNHLVRHRMLILDRLLKDLIQDFADGQKDRISRVCVEVGKELT
TFSAMDSKKIQRELTLRQKSHTDAVNRLKRKLPGKALSANLIRKCRIAMDMNWTCPFTGATYGD
HELENLELEHIVPHSFRQSNALSSLVLTWPGVNRMKGQRTGYDFVEQEQENPVPDKPNLHICSL
NNYRELVEKLDDKKGHEDDRRRKKKRKALLMVRGLSHKHQSQNHEAMKEIGMTEGMMTQSSHLM
KLACKSIKTSLPDAHIDMIPGAVTAEVRKAWDVFGVFKELCPEAADPDSGKILKENLRSLTHLH
HALDACVLGLIPYIIPAHHNGLLRRVLAMRRIPEKLIPQVRPVANQRHYVLNDDGRMMLRDLSA
SLKENIREQLMEQRVIQHVPADMGGALLKETMQRVLSVDGSGEDAMVSLSKKKDGKKEKNQVKA
SKLVGVFPEGPSKLKALKAAIEIDGNYGVALDPKPVVIRHIKVFKRIMALKEQNGGKPVRILKK
GMLIHLTSSKDPKHAGVWRIESIQDSKGGVKLDLQRAHCAVPKNKTHECNWREVDLISLLKKYQ
MKRYPTSYTGTPR
SEQ ID NO: 339
MTQKVLGLDLGTNSIGSAVRNLDLSDDLQWQLEFFSSDIFRSSVNKESNGREYSLAAQRSAHRR
SRGLNEVRRRRLWATLNLLIKHGFCPMSSESLMRWCTYDKRKGLFREYPIDDKDFNAWILLDFN
GDGRPDYSSPYQLRRELVTRQFDFEQPIERYKLGRALYHIAQHRGFKSSKGETLSQQETNSKPS
STDEIPDVAGAMKASEEKLSKGLSTYMKEHNLLTVGAAFAQLEDEGVRVRNNNDYRAIRSQFQH
EIETIFKFQQGLSVESELYERLISEKKNVGTIFYKRPLRSQRGNVGKCTLERSKPRCAIGHPLF
EKFRAWTLINNIKVRMSVDTLDEQLPMKLRLDLYNECFLAFVRTEFKFEDIRKYLEKRLGIHFS
YNDKTINYKDSTSVAGCPITARFRKMLGEEWESFRVEGQKERQAHSKNNISFHRVSYSIEDIWH
FCYDAEEPEAVLAFAQETLRLERKKAEELVRIWSAMPQGYAMLSQKAIRNINKILMLGLKYSDA
VILAKVPELVDVSDEELLSIAKDYYLVEAQVNYDKRINSIVNGLIAKYKSVSEEYRFADHNYEY
LLDESDEKDIIRQIENSLGARRWSLMDANEQTDILQKVRDRYQDFFRSHERKFVESPKLGESFE
NYLTKKFPMVEREQWKKLYHPSQITIYRPVSVGKDRSVLRLGNPDIGAIKNPTVLRVLNTLRRR
VNQLLDDGVISPDETRVVVETARELNDANRKWALDTYNRIRHDENEKIKKILEEFYPKRDGIST
DDIDKARYVIDQREVDYFTGSKTYNKDIKKYKFWLEQGGQCMYTGRTINLSNLFDPNAFDIEHT
IPESLSFDSSDMNLTLCDAHYNRFIKKNHIPTDMPNYDKAITIDGKEYPAITSQLQRWVERVER
LNRNVEYWKGQARRAQNKDRKDQCMREMHLWKMELEYWKKKLERFTVTEVTDGFKNSQLVDTRV
ITRHAVLYLKSIFPHVDVQRGDVTAKFRKILGIQSVDEKKDRSLHSHHAIDATTLTIIPVSAKR
DRMLELFAKIEEINKMLSFSGSEDRTGLIQELEGLKNKLQMEVKVCRIGHNVSEIGTFINDNII
VNHHIKNQALTPVRRRLRKKGYIVGGVDNPRWQTGDALRGEIHKASYYGAITQFAKDDEGKVLM
KEGRPQVNPTIKFVIRRELKYKKSAADSGFASWDDLGKAIVDKELFALMKGQFPAETSFKDACE
QGIYMIKKGKNGMPDIKLHHIRHVRCEAPQSGLKIKEQTYKSEKEYKRYFYAAVGDLYAMCCYT
NGKIREFRIYSLYDVSCHRKSDIEDIPEFITDKKGNRLMLDYKLRTGDMILLYKDNPAELYDLD
NVNLSRRLYKINRFESQSNLVLMTHHLSTSKERGRSLGKTVDYQNLPESIRSSVKSLNFLIMGE
NRDFVIKNGKIIFNHR
SEQ ID NO: 340
MLVSPISVDLGGKNTGFFSFTDSLDNSQSGTVIYDESFVLSQVGRRSKRHSKRNNLRNKLVKRL
FLLILQEHHGLSIDVLPDEIRGLFNKRGYTYAGFELDEKKKDALESDTLKEFLSEKLQSIDRDS
DVEDFLNQIASNAESFKDYKKGFEAVFASATHSPNKKLELKDELKSEYGENAKELLAGLRVTKE
ILDEFDKQENQGNLPRAKYFEELGEYIATNEKVKSFFDSNSLKLTDMTKLIGNISNYQLKELRR
YFNDKEMEKGDIWIPNKLHKITERFVRSWHPKNDADRQRRAELMKDLKSKEIMELLTTTEPVMT
IPPYDDMNNRGAVKCQTLRLNEEYLDKHLPNWRDIAKRLNHGKFNDDLADSTVKGYSEDSTLLH
RLLDTSKEIDIYELRGKKPNELLVKTLGQSDANRLYGFAQNYYELIRQKVRAGIWVPVKNKDDS
LNLEDNSNMLKRCNHNPPHKKNQIHNLVAGILGVKLDEAKFAEFEKELWSAKVGNKKLSAYCKN
IEELRKTHGNTFKIDIEELRKKDPAELSKEEKAKLRLTDDVILNEWSQKIANFFDIDDKHRQRF
NNLFSMAQLHTVIDTPRSGFSSTCKRCTAENRFRSETAFYNDETGEFHKKATATCQRLPADTQR
PFSGKIERYIDKLGYELAKIKAKELEGMEAKEIKVPIILEQNAFEYEESLRKSKTGSNDRVINS
KKDRDGKKLAKAKENAEDRLKDKDKRIKAFSSGICPYCGDTIGDDGEIDHILPRSHTLKIYGTV
FNPEGNLIYVHQKCNQAKADSIYKLSDIKAGVSAQWIEEQVANIKGYKTFSVLSAEQQKAFRYA
LFLQNDNEAYKKVVDWLRTDQSARVNGTQKYLAKKIQEKLTKMLPNKHLSFEFILADATEVSEL
RRQYARQNPLLAKAEKQAPSSHAIDAVMAFVARYQKVFKDGTPPNADEVAKLAMLDSWNPASNE
PLTKGLSTNQKIEKMIKSGDYGQKNMREVFGKSIFGENAIGERYKPIVVQEGGYYIGYPATVKK
GYELKNCKVVTSKNDIAKLEKIIKNQDLISLKENQYIKIFSINKQTISELSNRYFNMNYKNLVE
RDKEIVGLLEFIVENCRYYTKKVDVKFAPKYIHETKYPFYDDWRRFDEAWRYLQENQNKTSSKD
RFVIDKSSLNEYYQPDKNEYKLDVDTQPIWDDFCRWYFLDRYKTANDKKSIRIKARKTFSLLAE
SGVQGKVFRAKRKIPTGYAYQALPMDNNVIAGDYANILLEANSKTLSLVPKSGISIEKQLDKKL
DVIKKTDVRGLAIDNNSFFNADFDTHGIRLIVENTSVKVGNFPISAIDKSAKRMIFRALFEKEK
GKRKKKTTISFKESGPVQDYLKVFLKKIVKIQLRTDGSISNIVVRKNAADFTLSFRSEHIQKLL
K
SEQ ID NO: 341
MAYRLGLDIGITSVGWAVVALEKDESGLKPVRIQDLGVRIFDKAEDSKTGASLALPRREARSAR
RRTRRRRHRLWRVKRLLEQHGILSMEQIEALYAQRTSSPDVYALRVAGLDRCLIAEEIARVLIH
IAHRRGFQSNRKSEIKDSDAGKLLKAVQENENLMQSKGYRTVAEMLVSEATKTDAEGKLVHGKK
HGYVSNVRNKAGEYRHTVSRQAIVDEVRKIFAAQRALGNDVMSEELEDSYLKILCSQRNFDDGP
GGDSPYGHGSVSPDGVRQSIYERMVGSCTFETGEKRAPRSSYSFERFQLLTKVVNLRIYRQQED
GGRYPCELTQTERARVIDCAYEQTKITYGKLRKLLDMKDTESFAGLTYGLNRSRNKTEDTVFVE
MKFYHEVRKALQRAGVFIQDLSIETLDQIGWILSVWKSDDNRRKKLSTLGLSDNVIEELLPLNG
SKFGHLSLKAIRKILPFLEDGYSYDVACELAGYQFQGKTEYVKQRLLPPLGEGEVTNPVVRRAL
SQAIKVVNAVIRKHGSPESIHIELARELSKNLDERRKIEKAQKENQKNNEQIKDEIREILGSAH
VTGRDIVKYKLFKQQQEFCMYSGEKLDVTRLFEPGYAEVDHIIPYGISFDDSYDNKVLVKTEQN
RQKGNRTPLEYLRDKPEQKAKFIALVESIPLSQKKKNHLLMDKRAIDLEQEGFRERNLSDTRYI
TRALMNHIQAWLLFDETASTRSKRVVCVNGAVTAYMRARWGLTKDRDAGDKHHAADAVVVACIG
DSLIQRVTKYDKFKRNALADRNRYVQQVSKSEGITQYVDKETGEVFTWESFDERKFLPNEPLEP
WPFFRDELLARLSDDPSKNIRAIGLLTYSETEQIDPIFVSRMPTRKVTGAAHKETIRSPRIVKV
DDNKGTEIQVVVSKVALTELKLTKDGEIKDYFRPEDDPRLYNTLRERLVQFGGDAKAAFKEPVY
KISKDGSVRTPVRKVKIQEKLTLGVPVHGGRGIAENGGMVRIDVFAKGGKYYFVPIYVADVLKR
ELPNRLATAHKPYSEWRVVDDSYQFKFSLYPNDAVMIKPSREVDITYKDRKEPVGCRIMYFVSA
NIASASISLRTHDNSGELEGLGIQGLEVFEKYVVGPLGDTHPVYKERRMPFRVERKMN
SEQ ID NO: 342
MPVLSPLSPNAAQGRRRWSLALDIGEGSIGWAVAEVDAEGRVLQLTGTGVTLFPSAWSNENGTY
VAHGAADRAVRGQQQRHDSRRRRLAGLARLCAPVLERSPEDLKDLTRTPPKADPRAIFFLRADA
ARRPLDGPELFRVLHHMAAHRGIRLAELQEVDPPPESDADDAAPAATEDEDGTRRAAADERAFR
RLMAEHMHRHGTQPTCGEIMAGRLRETPAGAQPVTRARDGLRVGGGVAVPTRALIEQEFDAIRA
IQAPRHPDLPWDSLRRLVLDQAPIAVPPATPCLFLEELRRRGETFQGRTITREAIDRGLTVDPL
IQALRIRETVGNLRLHERITEPDGRQRYVPRAMPELGLSHGELTAPERDTLVRALMHDPDGLAA
KDGRIPYTRLRKLIGYDNSPVCFAQERDTSGGGITVNPTDPLMARWIDGWVDLPLKARSLYVRD
VVARGADSAALARLLAEGAHGVPPVAAAAVPAATAAILESDIMQPGRYSVCPWAAEAILDAWAN
APTEGFYDVTRGLFGFAPGEIVLEDLRRARGALLAHLPRTMAAARTPNRAAQQRGPLPAYESVI
PSQLITSLRRAHKGRAADWSAADPEERNPFLRTWTGNAATDHILNQVRKTANEVITKYGNRRGW
DPLPSRITVELAREAKHGVIRRNEIAKENRENEGRRKKESAALDTFCQDNTVSWQAGGLPKERA
ALRLRLAQRQEFFCPYCAERPKLRATDLFSPAETEIDHVIERRMGGDGPDNLVLAHKDCNNAKG
KKTPHEHAGDLLDSPALAALWQGWRKENADRLKGKGHKARTPREDKDFMDRVGWRFEEDARAKA
EENQERRGRRMLHDTARATRLARLYLAAAVMPEDPAEIGAPPVETPPSPEDPTGYTAIYRTISR
VQPVNGSVTHMLRQRLLQRDKNRDYQTHHAEDACLLLLAGPAVVQAFNTEAAQHGADAPDDRPV
DLMPTSDAYHQQRRARALGRVPLATVDAALADIVMPESDRQDPETGRVHWRLTRAGRGLKRRID
DLTRNCVILSRPRRPSETGTPGALHNATHYGRREITVDGRTDTVVTQRMNARDLVALLDNAKIV
PAARLDAAAPGDTILKEICTEIADRHDRVVDPEGTHARRWISARLAALVPAHAEAVARDIAELA
DLDALADADRTPEQEARRSALRQSPYLGRAISAKKADGRARAREQEILTRALLDPHWGPRGLRH
LIMREARAPSLVRIRANKTDAFGRPVPDAAVWVKTDGNAVSQLWRLTSVVTDDGRRIPLPKPIE
KRIEISNLEYARLNGLDEGAGVTGNNAPPRPLRQDIDRLTPLWRDHGTAPGGYLGTAVGELEDK
ARSALRGKAMRQTLTDAGITAEAGWRLDSEGAVCDLEVAKGDTVKKDGKTYKVGVITQGIFGMP
VDAAGSAPRTPEDCEKFEEQYGIKPWKAKGIPLA
SEQ ID NO: 343
MNYTEKEKLFMKYILALDIGIASVGWAILDKESETVIEAGSNIFPEASAADNQLRRDMRGAKRN
NRRLKTRINDFIKLWENNNLSIPQFKSTEIVGLKVRAITEEITLDELYLILYSYLKHRGISYLE
DALDDTVSGSSAYANGLKLNAKELETHYPCEIQQERLNTIGKYRGQSQIINENGEVLDLSNVFT
IGAYRKEIQRVFEIQKKYHPELTDEFCDGYMLIFNRKRKYYEGPGNEKSRTDYGRFTTKLDANG
NYITEDNIFEKLIGKCSVYPDELRAAAASYTAQEYNVLNDLNNLTINGRKLEENEKHEIVERIK
SSNTINMRKIISDCMGENIDDFAGARIDKSGKEIFHKFEVYNKMRKALLEIGIDISNYSREELD
EIGYIMTINTDKEAMMEAFQKSWIDLSDDVKQCLINMRKTNGALFNKWQSFSLKIMNELIPEMY
AQPKEQMTLLTEMGVTKGTQEEFAGLKYIPVDVVSEDIFNPVVRRSVRISFKILNAVLKKYKAL
DTIVIEMPRDRNSEEQKKRINDSQKLNEKEMEYIEKKLAVTYGIKLSPSDFSSQKQLSLKLKLW
NEQDGICLYSGKTIDPNDIINNPQLFEIDHIIPRSISFDDARSNKVLVYRSENQKKGNQTPYYY
LTHSHSEWSFEQYKATVMNLSKKKEYAISRKKIQNLLYSEDITKMDVLKGFINRNINDTSYASR
LVLNTIQNFFMANEADTKVKVIKGSYTHQMRCNLKLDKNRDESYSHHAVDAMLIGYSELGYEAY
HKLQGEFIDFETGEILRKDMWDENMSDEVYADYLYGKKWANIRNEVVKAEKNVKYWHYVMRKSN
RGLCNQTIRGTREYDGKQYKINKLDIRTKEGIKVFAKLAFSKKDSDRERLLVYLNDRRTFDDLC
KIYEDYSDAANPFVQYEKETGDIIRKYSKKHNGPRIDKLKYKDGEVGACIDISHKYGFEKGSKK
VILESLVPYRMDVYYKEENHSYYLVGVKQSDIKFEKGRNVIDEEAYARILVNEKMIQPGQSRAD
LENLGFKFKLSFYKNDIIEYEKDGKIYTERLVSRTMPKQRNYIETKPIDKAKFEKQNLVGLGKT
KFIKKYRYDILGNKYSCSEEKFTSFC
SEQ ID NO: 344
MLRLYCANNLVLNNVQNLWKYLLLLIFDKKIIFLFKIKVILIRRYMENNNKEKIVIGFDLGVAS
VGWSIVNAETKEVIDLGVRLFSEPEKADYRRAKRTTRRLLRRKKFKREKFHKLILKNAEIFGLQ
SRNEILNVYKDQSSKYRNILKLKINALKEEIKPSELVWILRDYLQNRGYFYKNEKLTDEFVSNS
FPSKKLHEHYEKYGFFRGSVKLDNKLDNKKDKAKEKDEEEESDAKKESEELIFSNKQWINEIVK
VFENQSYLTESFKEEYLKLFNYVRPFNKGPGSKNSRTAYGVFSTDIDPETNKFKDYSNIWDKTI
GKCSLFEEEIRAPKNLPSALIFNLQNEICTIKNEFTEFKNWWLNAEQKSEILKFVFTELFNWKD
KKYSDKKFNKNLQDKIKKYLLNFALENFNLNEEILKNRDLENDTVLGLKGVKYYEKSNATADAA
LEFSSLKPLYVFIKFLKEKKLDLNYLLGLENTEILYFLDSIYLAISYSSDLKERNEWFKKLLKE
LYPKIKNNNLEIIENVEDIFEITDQEKFESFSKTHSLSREAFNHIIPLLLSNNEGKNYESLKHS
NEELKKRTEKAELKAQQNQKYLKDNFLKEALVPLSVKTSVLQAIKIFNQIIKNFGKKYEISQVV
IEMARELTKPNLEKLLNNATNSNIKILKEKLDQTEKFDDFTKKKFIDKIENSVVFRNKLFLWFE
QDRKDPYTQLDIKINEIEDETEIDHVIPYSKSADDSWFNKLLVKKSTNQLKKNKTVWEYYQNES
DPEAKWNKFVAWAKRIYLVQKSDKESKDNSEKNSIFKNKKPNLKFKNITKKLFDPYKDLGFLAR
NLNDTRYATKVFRDQLNNYSKHHSKDDENKLFKVVCMNGSITSFLRKSMWRKNEEQVYRFNFWK
KDRDQFFHHAVDASIIAIFSLLTKTLYNKLRVYESYDVQRREDGVYLINKETGEVKKADKDYWK
DQHNFLKIRENAIEIKNVLNNVDFQNQVRYSRKANTKLNTQLFNETLYGVKEFENNFYKLEKVN
LFSRKDLRKFILEDLNEESEKNKKNENGSRKRILTEKYIVDEILQILENEEFKDSKSDINALNK
YMDSLPSKFSEFFSQDFINKCKKENSLILTFDAIKHNDPKKVIKIKNLKFFREDATLKNKQAVH
KDSKNQIKSFYESYKCVGFIWLKNKNDLEESIFVPINSRVIHFGDKDKDIFDFDSYNKEKLLNE
INLKRPENKKFNSINEIEFVKFVKPGALLLNFENQQIYYISTLESSSLRAKIKLLNKMDKGKAV
SMKKITNPDEYKIIEHVNPLGINLNWTKKLENNN
SEQ ID NO: 345
MLMSKHVLGLDLGVGSIGWCLIALDAQGDPAEILGMGSRVVPLNNATKAIEAFNAGAAFTASQE
RTARRTMRRGFARYQLRRYRLRRELEKVGMLPDAALIQLPLLELWELRERAATAGRRLTLPELG
RVLCHINQKRGYRHVKSDAAAIVGDEGEKKKDSNSAYLAGIRANDEKLQAEHKTVGQYFAEQLR
QNQSESPTGGISYRIKDQIFSRQCYIDEYDQIMAVQRVHYPDILTDEFIRMLRDEVIFMQRPLK
SCKHLVSLCEFEKQERVMRVQQDDGKGGWQLVERRVKFGPKVAPKSSPLFQLCCIYEAVNNIRL
TRPNGSPCDITPEERAKIVAHLQSSASLSFAALKKLLKEKALIADQLTSKSGLKGNSTRVALAS
ALQPYPQYHHLLDMELETRMMTVQLTDEETGEVTEREVAVVTDSYVRKPLYRLWHILYSIEERE
AMRRALITQLGMKEEDLDGGLLDQLYRLDFVKPGYGNKSAKFICKLLPQLQQGLGYSEACAAVG
YRHSNSPTSEEITERTLLEKIPLLQRNELRQPLVEKILNQMINLVNALKAEYGIDEVRVELARE
LKMSREERERMARNNKDREERNKGVAAKIRECGLYPTKPRIQKYMLWKEAGRQCLYCGRSIEEE
QCLREGGMEVEHIIPKSVLYDDSYGNKTCACRRCNKEKGNRTALEYIRAKGREAEYMKRINDLL
KEKKISYSKHQRLRWLKEDIPSDFLERQLRLTQYISRQAMAILQQGIRRVSASEGGVTARLRSL
WGYGKILHTLNLDRYDSMGETERVSREGEATEELHITNWSKRMDHRHHAIDALVVACTRQSYIQ
RLNRLSSEFGREDKKKEDQEAQEQQATETGRLSNLERWLTQRPHFSVRTVSDKVAEILISYRPG
QRVVTRGRNIYRKKMADGREVSCVQRGVLVPRGELMEASFYGKILSQGRVRIVKRYPLHDLKGE
VVDPHLRELITTYNQELKSREKGAPIPPLCLDKDKKQEVRSVRCYAKTLSLDKAIPMCFDEKGE
PTAFVKSASNHHLALYRTPKGKLVESIVTFWDAVDRARYGIPLVITHPREVMEQVLQRGDIPEQ
VLSLLPPSDWVFVDSLQQDEMVVIGLSDEELQRALEAQNYRKISEHLYRVQKMSSSYYVFRYHL
ETSVADDKNTSGRIPKFHRVQSLKAYEERNIRKVRVDLLGRISLL
SEQ ID NO: 346
MSDLVLGLDIGIGSVGVGILNKVTGEIIHKNSRIFPAAQAENNLVRRTNRQGRRLARRKKHRRV
RLNRLFEESGLITDFTKISINLNPYQLRVKGLTDELSNEELFIALKNMVKHRGISYLDDASDDG
NSSVGDYAQIVKENSKQLETKTPGQIQLERYQTYGQLRGDFTVEKDGKKHRLINVFPTSAYRSE
ALRILQTQQEFNPQITDEFINRYLEILTGKRKYYHGPGNEKSRTDYGRYRTSGETLDNIFGILI
GKCTFYPDEFRAAKASYTAQEFNLLNDLNNLTVPTETKKLSKEQKNQIINYVKNEKAMGPAKLF
KYIAKLLSCDVADIKGYRIDKSGKAEIHTFEAYRKMKTLETLDIEQMDRETLDKLAYVLTLNTE
REGIQEALEHEFADGSFSQKQVDELVQFRKANSSIFGKGWHNFSVKLMMELIPELYETSEEQMT
ILTRLGKQKTTSSSNKTKYIDEKLLTEEIYNPVVAKSVRQAIKIVNAAIKEYGDFDNIVIEMAR
ETNEDDEKKAIQKIQKANKDEKDAAMLKAANQYNGKAELPHSVFHGHKQLATKIRLWHQQGERC
LYTGKTISIHDLINNSNQFEVDHILPLSITFDDSLANKVLVYATANQEKGQRTPYQALDSMDDA
WSFRELKAFVRESKTLSNKKKEYLLTEEDISKFDVRKKFIERNLVDTRYASRVVLNALQEHFRA
HKIDTKVSVVRGQFTSQLRRHWGIEKTRDTYHHHAVDALIIAASSQLNLWKKQKNTLVSYSEDQ
LLDIETGELISDDEYKESVFKAPYQHFVDTLKSKEFEDSILFSYQVDSKFNRKISDATIYATRQ
AKVGKDKADETYVLGKIKDIYTQDGYDAFMKIYKKDKSKFLMYRHDPQTFEKVIEPILENYPNK
QINEKGKEVPCNPFLKYKEEHGYIRKYSKKGNGPEIKSLKYYDSKLGNHIDITPKDSNNKVVLQ
SVSPWRADVYFNKTTGKYEILGLKYADLQFEKGTGTYKISQEKYNDIKKKEGVDSDSEFKFTLY
KNDLLLVKDTETKEQQLFRFLSRTMPKQKHYVELKPYDKQKFEGGEALIKVLGNVANSGQCKKG
LGKSNISIYKVRTDVLGNQHIIKNEGDKPKLDF
SEQ ID NO: 347
MNAEHGKEGLLIMEENFQYRIGLDIGITSVGWAVLQNNSQDEPVRITDLGVRIFDVAENPKNGD
ALAAPRRDARTTRRRLRRRRHRLERIKFLLQENGLIEMDSFMERYYKGNLPDVYQLRYEGLDRK
LKDEELAQVLIHIAKHRGFRSTRKAETKEKEGGAVLKATTENQKIMQEKGYRTVGEMLYLDEAF
HTECLWNEKGYVLTPRNRPDDYKHTILRSMLVEEVHAIFAAQRAHGNQKATEGLEEAYVEIMTS
QRSFDMGPGLQPDGKPSPYAMEGFGDRVGKCTFEKDEYRAPKATYTAELFVALQKINHTKLIDE
FGTGRFFSEEERKTIIGLLLSSKELKYGTIRKKLNIDPSLKFNSLNYSAKKEGETEEERVLDTE
KAKFASMFWTYEYSKCLKDRTEEMPVGEKADLFDRIGEILTAYKNDDSRSSRLKELGLSGEEID
GLLDLSPAKYQRVSLKAMRKMQPYLEDGLIYDKACEAAGYDFRALNDGNKKHLLKGEEINAIVN
DITNPVVKRSVSQTIKVINAIIQKYGSPQAVNIELAREMSKNFQDRTNLEKEMKKRQQENERAK
QQIIELGKQNPTGQDILKYRLWNDQGGYCLYSGKKIPLEELFDGGYDIDHILPYSITFDDSYRN
KVLVTAQENRQKGNRTPYEYFGADEKRWEDYEASVRLLVRDYKKQQKLLKKNFTEEERKEFKER
NLNDTKYITRVVYNMIRQNLELEPFNHPEKKKQVWAVNGAVTSYLRKRWGLMQKDRSTDRHHAM
DAVVIACCTDGMIHKISRYMQGRELAYSRNFKFPDEETGEILNRDNFTREQWDEKFGVKVPLPW
NSFRDELDIRLLNEDPKNFLLTHADVQRELDYPGWMYGEEESPIEEGRYINYIRPLFVSRMPNH
KVTGSAHDATIRSARDYETRGVVITKVPLTDLKLNKDNEIEGYYDKDSDRLLYQALVRQLLLHG
NDGKKAFAEDFHKPKADGTEGPVVRKVKIEKKQTSGVMVRGGTGIAANGEMVRIDVFRENGKYY
FVPVYTADVVRKVLPNRAATHTKPYSEWRVMDDANFVFSLYSRDLIHVKSKKDIKTNLVNGGLL
LQKEIFAYYTGADIATASIAGFANDSNFKFRGLGIQSLEIFEKCQVDILGNISVVRHENRQEFH
SEQ ID NO: 348
MRVLGLDAGIASLGWALIEIEESNRGELSQGTIIGAGTWMFDAPEEKTQAGAKLKSEQRRTFRG
QRRVVRRRRQRMNEVRRILHSHGLLPSSDRDALKQPGLDPWRIRAEALDRLLGPVELAVALGHI
ARHRGFKSNSKGAKTNDPADDTSKMKRAVNETREKLARFGSAAKMLVEDESFVLRQTPTKNGAS
EIVRRFRNREGDYSRSLLRDDLAAEMRALFTAQARFQSAIATADLQTAFTKAAFFQRPLQDSEK
LVGPCPFEVDEKRAPKRGYSFELFRFLSRLNHVTLRDGKQERTLTRDELALAAADFGAAAKVSF
TALRKKLKLPETTVFVGVKADEESKLDVVARSGKAAEGTARLRSVIVDALGELAWGALLCSPEK
LDKIAEVISFRSDIGRISEGLAQAGCNAPLVDALTAAASDGRFDPFTGAGHISSKAARNILSGL
RQGMTYDKACCAADYDHTASRERGAFDVGGHGREALKRILQEERISRELVGSPTARKALIESIK
QVKAIVERYGVPDRIHVELARDVGKSIEEREEITRGIEKRNRQKDKLRGLFEKEVGRPPQDGAR
GKEELLRFELWSEQMGRCLYTDDYISPSQLVATDDAVQVDHILPWSRFADDSYANKTLCMAKAN
QDKKGRTPYEWFKAEKTDTEWDAFIVRVEALADMKGFKKRNYKLRNAEEAAAKFRNRNLNDTRW
ACRLLAEALKQLYPKGEKDKDGKERRRVFSRPGALTDRLRRAWGLQWMKKSTKGDRIPDDRHHA
LDAIVIAATTESLLQRATREVQEIEDKGLHYDLVKNVTPPWPGFREQAVEAVEKVFVARAERRR
ARGKAHDATIRHIAVREGEQRVYERRKVAELKLADLDRVKDAERNARLIEKLRNWIEAGSPKDD
PPLSPKGDPIFKVRLVTKSKVNIALDTGNPKRPGTVDRGEMARVDVFRKASKKGKYEYYLVPIY
PHDIATMKTPPIRAVQAYKPEDEWPEMDSSYEFCWSLVPMTYLQVISSKGEIFEGYYRGMNRSV
GAIQLSAHSNSSDVVQGIGARTLTEFKKFNVDRFGRKHEVERELRTWRGETWRGKAYI
SEQ ID NO: 349
MGNYYLGLDVGIGSIGWAVINIEKKRIEDFNVRIFKSGEIQEKNRNSRASQQCRRSRGLRRLYR
RKSHRKLRLKNYLSIIGLTTSEKIDYYYETADNNVIQLRNKGLSEKLTPEEIAACLIHICNNRG
YKDFYEVNVEDIEDPDERNEYKEEHDSIVLISNLMNEGGYCTPAEMICNCREFDEPNSVYRKFH
NSAASKNHYLITRHMLVKEVDLILENQSKYYGILDDKTIAKIKDIIFAQRDFEIGPGKNERFRR
FTGYLDSIGKCQFFKDQERGSRFTVIADIYAFVNVLSQYTYTNNRGESVFDTSFANDLINSALK
NGSMDKRELKAIAKSYHIDISDKNSDTSLTKCFKYIKVVKPLFEKYGYDWDKLIENYTDTDNNV
LNRIGIVLSQAQTPKRRREKLKALNIGLDDGLINELTKLKLSGTANVSYKYMQGSIEAFCEGDL
YGKYQAKFNKEIPDIDENAKPQKLPPFKNEDDCEFFKNPVVFRSINETRKLINAIIDKYGYPAA
VNIETADELNKTFEDRAIDTKRNNDNQKENDRIVKEIIECIKCDEVHARHLIEKYKLWEAQEGK
CLYSGETITKEDMLRDKDKLFEVDHIVPYSLILDNTINNKALVYAEENQKKGQRTPLMYMNEAQ
AADYRVRVNTMFKSKKCSKKKYQYLMLPDLNDQELLGGWRSRNLNDTRYICKYLVNYLRKNLRF
DRSYESSDEDDLKIRDHYRVFPVKSRFTSMFRRWWLNEKTWGRYDKAELKKLTYLDHAADAIII
ANCRPEYVVLAGEKLKLNKMYHQAGKRITPEYEQSKKACIDNLYKLFRMDRRTAEKLLSGHGRL
TPIIPNLSEEVDKRLWDKNIYEQFWKDDKDKKSCEELYRENVASLYKGDPKFASSLSMPVISLK
PDHKYRGTITGEEAIRVKEIDGKLIKLKRKSISEITAESINSIYTDDKILIDSLKTIFEQADYK
DVGDYLKKTNQHFFTTSSGKRVNKVTVIEKVPSRWLRKEIDDNNFSLLNDSSYYCIELYKDSKG
DNNLQGIAMSDIVHDRKTKKLYLKPDFNYPDDYYTHVMYIFPGDYLRIKSTSKKSGEQLKFEGY
FISVKNVNENSFRFISDNKPCAKDKRVSITKKDIVIKLAVDLMGKVQGENNGKGISCGEPLSLL
KEKN
SEQ ID NO: 350
MLSRQLLGASHLARPVSYSYNVQDNDVHCSYGERCFMRGKRYRIGIDVGLNSVGLAAVEVSDEN
SPVRLLNAQSVIHDGGVDPQKNKEAITRKNMSGVARRTRRMRRRKRERLHKLDMLLGKFGYPVI
EPESLDKPFEEWHVRAELATRYIEDDELRRESISIALRHMARHRGWRNPYRQVDSLISDNPYSK
QYGELKEKAKAYNDDATAAEEESTPAQLVVAMLDAGYAEAPRLRWRTGSKKPDAEGYLPVRLMQ
EDNANELKQIFRVQRVPADEWKPLFRSVFYAVSPKGSAEQRVGQDPLAPEQARALKASLAFQEY
RIANVITNLRIKDASAELRKLTVDEKQSIYDQLVSPSSEDITWSDLCDFLGFKRSQLKGVGSLT
EDGEERISSRPPRLTSVQRIYESDNKIRKPLVAWWKSASDNEHEAMIRLLSNTVDIDKVREDVA
YASAIEFIDGLDDDALTKLDSVDLPSGRAAYSVETLQKLTRQMLTTDDDLHEARKTLFNVTDSW
RPPADPIGEPLGNPSVDRVLKNVNRYLMNCQQRWGNPVSVNIEHVRSSFSSVAFARKDKREYEK
NNEKRSIFRSSLSEQLRADEQMEKVRESDLRRLEAIQRQNGQCLYCGRTITFRTCEMDHIVPRK
GVGSTNTRTNFAAVCAECNRMKSNTPFAIWARSEDAQTRGVSLAEAKKRVTMFTFNPKSYAPRE
VKAFKQAVIARLQQTEDDAAIDNRSIESVAWMADELHRRIDWYFNAKQYVNSASIDDAEAETMK
TTVSVFQGRVTASARRAAGIEGKIHFIGQQSKTRLDRRHHAVDASVIAMMNTAAAQTLMERESL
RESQRLIGLMPGERSWKEYPYEGTSRYESFHLWLDNMDVLLELLNDALDNDRIAVMQSQRYVLG
NSIAHDATIHPLEKVPLGSAMSADLIRRASTPALWCALTRLPDYDEKEGLPEDSHREIRVHDTR
YSADDEMGFFASQAAQIAVQEGSADIGSAIHHARVYRCWKTNAKGVRKYFYGMIRVFQTDLLRA
CHDDLFTVPLPPQSISMRYGEPRVVQALQSGNAQYLGSLVVGDEIEMDFSSLDVDGQIGEYLQF
FSQFSGGNLAWKHWVVDGFFNQTQLRIRPRYLAAEGLAKAFSDDVVPDGVQKIVTKQGWLPPVN
TASKTAVRIVRRNAFGEPRLSSAHHMPCSWQWRHE
SEQ ID NO: 351
MYSIGLDLGISSVGWSVIDERTGNVIDLGVRLFSAKNSEKNLERRTNRGGRRLIRRKTNRLKDA
KKILAAVGFYEDKSLKNSCPYQLRVKGLTEPLSRGEIYKVTLHILKKRGISYLDEVDTEAAKES
QDYKEQVRKNAQLLTKYTPGQIQLQRLKENNRVKTGINAQGNYQLNVFKVSAYANELATILKTQ
QAFYPNELTDDWIALFVQPGIAEEAGLIYRKRPYYHGPGNEANNSPYGRWSDFQKTGEPATNIF
DKLIGKDFQGELRASGLSLSAQQYNLLNDLTNLKIDGEVPLSSEQKEYILTELMTKEFTRFGVN
DVVKLLGVKKERLSGWRLDKKGKPEIHTLKGYRNWRKIFAEAGIDLATLPTETIDCLAKVLTLN
TEREGIENTLAFELPELSESVKLLVLDRYKELSQSISTQSWHRFSLKTLHLLIPELMNATSEQN
TLLEQFQLKSDVRKRYSEYKKLPTKDVLAEIYNPTVNKTVSQAFKVIDALLVKYGKEQIRYITI
EMPRDDNEEDEKKRIKELHAKNSQRKNDSQSYFMQKSGWSQEKFQTTIQKNRRFLAKLLYYYEQ
DGICAYTGLPISPELLVSDSTEIDHIIPISISLDDSINNKVLVLSKANQVKGQQTPYDAWMDGS
FKKINGKFSNWDDYQKWVESRHFSHKKENNLLETRNIFDSEQVEKFLARNLNDTRYASRLVLNT
LQSFFTNQETKVRVVNGSFTHTLRKKWGADLDKTRETHHHHAVDATLCAVTSFVKVSRYHYAVK
EETGEKVMREIDFETGEIVNEMSYWEFKKSKKYERKTYQVKWPNFREQLKPVNLHPRIKFSHQV
DRKANRKLSDATIYSVREKTEVKTLKSGKQKITTDEYTIGKIKDIYTLDGWEAFKKKQDKLLMK
DLDEKTYERLLSIAETTPDFQEVEEKNGKVKRVKRSPFAVYCEENDIPAIQKYAKKNNGPLIRS
LKYYDGKLNKHINITKDSQGRPVEKTKNGRKVTLQSLKPYRYDIYQDLETKAYYTVQLYYSDLR
FVEGKYGITEKEYMKKVAEQTKGQVVRFCFSLQKNDGLEIEWKDSQRYDVRFYNFQSANSINFK
GLEQEMMPAENQFKQKPYNNGAINLNIAKYGKEGKKLRKFNTDILGKKHYLFYEKEPKNIIK
SEQ ID NO: 352
MYFYKNKENKLNKKVVLGLDLGIASVGWCLTDISQKEDNKFPIILHGVRLFETVDDSDDKLLNE
TRRKKRGQRRRNRRLFTRKRDFIKYLIDNNIIELEFDKNPKILVRNFIEKYINPFSKNLELKYK
SVTNLPIGFHNLRKAAINEKYKLDKSELIVLLYFYLSLRGAFFDNPEDTKSKEMNKNEIEIFDK
NESIKNAEFPIDKIIEFYKISGKIRSTINLKFGHQDYLKEIKQVFEKQNIDFMNYEKFAMEEKS
FFSRIRNYSEGPGNEKSFSKYGLYANENGNPELIINEKGQKIYTKIFKTLWESKIGKCSYDKKL
YRAPKNSFSAKVFDITNKLTDWKHKNEYISERLKRKILLSRFLNKDSKSAVEKILKEENIKFEN
LSEIAYNKDDNKINLPIINAYHSLTTIFKKHLINFENYLISNENDLSKLMSFYKQQSEKLFVPN
EKGSYEINQNNNVLHIFDAISNILNKFSTIQDRIRILEGYFEFSNLKKDVKSSEIYSEIAKLRE
FSGTSSLSFGAYYKFIPNLISEGSKNYSTISYEEKALQNQKNNFSHSNLFEKTWVEDLIASPTV
KRSLRQTMNLLKEIFKYSEKNNLEIEKIVVEVTRSSNNKHERKKIEGINKYRKEKYEELKKVYD
LPNENTTLLKKLWLLRQQQGYDAYSLRKIEANDVINKPWNYDIDHIVPRSISFDDSFSNLVIVN
KLDNAKKSNDLSAKQFIEKIYGIEKLKEAKENWGNWYLRNANGKAFNDKGKFIKLYTIDNLDEF
DNSDFINRNLSDTSYITNALVNHLTFSNSKYKYSVVSVNGKQTSNLRNQIAFVGIKNNKETERE
WKRPEGFKSINSNDFLIREEGKNDVKDDVLIKDRSFNGHHAEDAYFITIISQYFRSFKRIERLN
VNYRKETRELDDLEKNNIKFKEKASFDNFLLINALDELNEKLNQMRFSRMVITKKNTQLFNETL
YSGKYDKGKNTIKKVEKLNLLDNRTDKIKKIEEFFDEDKLKENELTKLHIFNHDKNLYETLKII
WNEVKIEIKNKNLNEKNYFKYFVNKKLQEGKISFNEWVPILDNDFKIIRKIRYIKFSSEEKETD
EIIFSQSNFLKIDQRQNFSFHNTLYWVQIWVYKNQKDQYCFISIDARNSKFEKDEIKINYEKLK
TQKEKLQIINEEPILKINKGDLFENEEKELFYIVGRDEKPQKLEIKYILGKKIKDQKQIQKPVK
KYFPNWKKVNLTYMGEIFKK
SEQ ID NO: 353
MDNKNYRIGIDVGLNSIGFCAVEVDQHDTPLGFLNLSVYRHDAGIDPNGKKTNTTRLAMSGVAR
RTRRLFRKRKRRLAALDRFIEAQGWTLPDHADYKDPYTPWLVRAELAQTPIRDENDLHEKLAIA
VRHIARHRGWRSPWVPVRSLHVEQPPSDQYLALKERVEAKTLLQMPEGATPAEMVVALDLSVDV
NLRPKNREKTDTRPENKKPGFLGGKLMQSDNANELRKIAKIQGLDDALLRELIELVFAADSPKG
ASGELVGYDVLPGQHGKRRAEKAHPAFQRYRIASIVSNLRIRHLGSGADERLDVETQKRVFEYL
LNAKPTADITWSDVAEEIGVERNLLMGTATQTADGERASAKPPVDVTNVAFATCKIKPLKEWWL
NADYEARCVMVSALSHAEKLTEGTAAEVEVAEFLQNLSDEDNEKLDSFSLPIGRAAYSVDSLER
LTKRMIENGEDLFEARVNEFGVSEDWRPPAEPIGARVGNPAVDRVLKAVNRYLMAAEAEWGAPL
SVNIEHVREGFISKRQAVEIDRENQKRYQRNQAVRSQIADHINATSGVRGSDVTRYLAIQRQNG
ECLYCGTAITFVNSEMDHIVPRAGLGSTNTRDNLVATCERCNKSKSNKPFAVWAAECGIPGVSV
AEALKRVDFWIADGFASSKEHRELQKGVKDRLKRKVSDPEIDNRSMESVAWMARELAHRVQYYF
DEKHTGTKVRVFRGSLTSAARKASGFESRVNFIGGNGKTRLDRRHHAMDAATVAMLRNSVAKTL
VLRGNIRASERAIGAAETWKSFRGENVADRQIFESWSENMRVLVEKFNLALYNDEVSIFSSLRL
QLGNGKAHDDTITKLQMHKVGDAWSLTEIDRASTPALWCALTRQPDFTWKDGLPANEDRTIIVN
GTHYGPLDKVGIFGKAAASLLVRGGSVDIGSAIHHARIYRIAGKKPTYGMVRVFAPDLLRYRNE
DLFNVELPPQSVSMRYAEPKVREAIREGKAEYLGWLVVGDELLLDLSSETSGQIAELQQDFPGT
THWTVAGFFSPSRLRLRPVYLAQEGLGEDVSEGSKSIIAGQGWRPAVNKVFGSAMPEVIRRDGL
GRKRRFSYSGLPVSWQG
SEQ ID NO: 354
MRLGLDIGTSSIGWWLYETDGAGSDARITGVVDGGVRIFSDGRDPKSGASLAVDRRAARAMRRR
RDRYLRRRATLMKVLAETGLMPADPAEAKALEALDPFALRAAGLDEPLPLPHLGRALFHLNQRR
GFKSNRKTDRGDNESGKIKDATARLDMEMMANGARTYGEFLHKRRQKATDPRHVPSVRTRLSIA
NRGGPDGKEEAGYDFYPDRRHLEEEFHKLWAAQGAHHPELTETLRDLLFEKIFFQRPLKEPEVG
LCLFSGHHGVPPKDPRLPKAHPLTQRRVLYETVNQLRVTADGREARPLTREERDQVIHALDNKK
PTKSLSSMVLKLPALAKVLKLRDGERFTLETGVRDAIACDPLRASPAHPDRFGPRWSILDADAQ
WEVISRIRRVQSDAEHAALVDWLTEAHGLDRAHAEATAHAPLPDGYGRLGLTATTRILYQLTAD
VVTYADAVKACGWHHSDGRTGECFDRLPYYGEVLERHVIPGSYHPDDDDITRFGRITNPTVHIG
LNQLRRLVNRIIETHGKPHQIVVELARDLKKSEEQKRADIKRIRDTTEAAKKRSEKLEELEIED
NGRNRMLLRLWEDLNPDDAMRRFCPYTGTRISAAMIFDGSCDVDHILPYSRTLDDSFPNRTLCL
REANRQKRNQTPWQAWGDTPHWHAIAANLKNLPENKRWRFAPDAMTRFEGENGFLDRALKDTQY
LARISRSYLDTLFTKGGHVWVVPGRFTEMLRRHWGLNSLLSDAGRGAVKAKNRTDHRHHAIDAA
VIAATDPGLLNRISRAAGQGEAAGQSAELIARDTPPPWEGFRDDLRVRLDRIIVSHRADHGRID
HAARKQGRDSTAGQLHQETAYSIVDDIHVASRTDLLSLKPAQLLDEPGRSGQVRDPQLRKALRV
ATGGKTGKDFENALRYFASKPGPYQAIRRVRIIKPLQAQARVPVPAQDPIKAYQGGSNHLFEIW
RLPDGEIEAQVITSFEAHTLEGEKRPHPAAKRLLRVHKGDMVALERDGRRVVGHVQKMDIANGL
FIVPHNEANADTRNNDKSDPFKWIQIGARPAIASGIRRVSVDEIGRLRDGGTRPI
SEQ ID NO: 355
MLHCIAVIRVPPSEEPGFFETHADSCALCHHGCMTYAANDKAIRYRVGIDVGLRSIGFCAVEVD
DEDHPIRILNSVVHVHDAGTGGPGETESLRKRSGVAARARRRGRAEKQRLKKLDVLLEELGWGV
SSNELLDSHAPWHIRKRLVSEYIEDETERRQCLSVAMAHIARHRGWRNSFSKVDTLLLEQAPSD
RMQGLKERVEDRTGLQFSEEVTQGELVATLLEHDGDVTIRGFVRKGGKATKVHGVLEGKYMQSD
LVAELRQICRTQRVSETTFEKLVLSIFHSKEPAPSAARQRERVGLDELQLALDPAAKQPRAERA
HPAFQKFKVVATLANMRIREQSAGERSLTSEELNRVARYLLNHTESESPTWDDVARKLEVPRHR
LRGSSRASLETGGGLTYPPVDDTTVRVMSAEVDWLADWWDCANDESRGHMIDAISNGCGSEPDD
VEDEEVNELISSATAEDMLKLELLAKKLPSGRVAYSLKTLREVTAAILETGDDLSQAITRLYGV
DPGWVPTPAPIEAPVGNPSVDRVLKQVARWLKFASKRWGVPQTVNIEHTREGLKSASLLEEERE
RWERFEARREIRQKEMYKRLGISGPFRRSDQVRYEILDLQDCACLYCGNEINFQTFEVDHIIPR
VDASSDSRRTNLAAVCHSCNSAKGGLAFGQWVKRGDCPSGVSLENAIKRVRSWSKDRLGLTEKA
MGKRKSEVISRLKTEMPYEEFDGRSMESVAWMAIELKKRIEGYFNSDRPEGCAAVQVNAYSGRL
TACARRAAHVDKRVRLIRLKGDDGHHKNRFDRRNHAMDALVIALMTPAIARTIAVREDRREAQQ
LTRAFESWKNFLGSEERMQDRWESWIGDVEYACDRLNELIDADKIPVTENLRLRNSGKLHADQP
ESLKKARRGSKRPRPQRYVLGDALPADVINRVTDPGLWTALVRAPGFDSQLGLPADLNRGLKLR
GKRISADFPIDYFPTDSPALAVQGGYVGLEFHHARLYRIIGPKEKVKYALLRVCAIDLCGIDCD
DLFEVELKPSSISMRTADAKLKEAMGNGSAKQIGWLVLGDEIQIDPTKFPKQSIGKFLKECGPV
SSWRVSALDTPSKITLKPRLLSNEPLLKTSRVGGHESDLVVAECVEKIMKKTGWVVEINALCQS
GLIRVIRRNALGEVRTSPKSGLPISLNLR
SEQ ID NO: 356
MRYRVGLDLGTASVGAAVFSMDEQGNPMELIWHYERLFSEPLVPDMGQLKPKKAARRLARQQRR
QIDRRASRLRRIAIVSRRLGIAPGRNDSGVHGNDVPTLRAMAVNERIELGQLRAVLLRMGKKRG
YGGTFKAVRKVGEAGEVASGASRLEEEMVALASVQNKDSVTVGEYLAARVEHGLPSKLKVAANN
EYYAPEYALFRQYLGLPAIKGRPDCLPNMYALRHQIEHEFERIWATQSQFHDVMKDHGVKEEIR
NAIFFQRPLKSPADKVGRCSLQTNLPRAPRAQIAAQNFRIEKQMADLRWGMGRRAEMLNDHQKA
VIRELLNQQKELSFRKIYKELERAGCPGPEGKGLNMDRAALGGRDDLSGNTTLAAWRKLGLEDR
WQELDEVTQIQVINFLADLGSPEQLDTDDWSCRFMGKNGRPRNFSDEFVAFMNELRMTDGFDRL
SKMGFEGGRSSYSIKALKALTEWMIAPHWRETPETHRVDEEAAIRECYPESLATPAQGGRQSKL
EPPPLTGNEVVDVALRQVRHTINMMIDDLGSVPAQIVVEMAREMKGGVTRRNDIEKQNKRFASE
RKKAAQSIEENGKTPTPARILRYQLWIEQGHQCPYCESNISLEQALSGAYTNFEHILPRTLTQI
GRKRSELVLAHRECNDEKGNRTPYQAFGHDDRRWRIVEQRANALPKKSSRKTRLLLLKDFEGEA
LTDESIDEFADRQLHESSWLAKVTTQWLSSLGSDVYVSRGSLTAELRRRWGLDTVIPQVRFESG
MPVVDEEGAEITPEEFEKFRLQWEGHRVTREMRTDRRPDKRIDHRHHLVDAIVTALTSRSLYQQ
YAKAWKVADEKQRHGRVDVKVELPMPILTIRDIALEAVRSVRISHKPDRYPDGRFFEATAYGIA
QRLDERSGEKVDWLVSRKSLTDLAPEKKSIDVDKVRANISRIVGEAIRLHISNIFEKRVSKGMT
PQQALREPIEFQGNILRKVRCFYSKADDCVRIEHSSRRGHHYKMLLNDGFAYMEVPCKEGILYG
VPNLVRPSEAVGIKRAPESGDFIRFYKGDTVKNIKTGRVYTIKQILGDGGGKLILTPVTETKPA
DLLSAKWGRLKVGGRNIHLLRLCAE
SEQ ID NO: 357
MIGEHVRGGCLFDDHWTPNWGAFRLPNTVRTFTKAENPKDGSSLAEPRRQARGLRRRLRRKTQR
LEDLRRLLAKEGVLSLSDLETLFRETPAKDPYQLRAEGLDRPLSFPEWVRVLYHITKHRGFQSN
RRNPVEDGQERSRQEEEGKLLSGVGENERLLREGGYRTAGEMLARDPKFQDHRRNRAGDYSHTL
SRSLLLEEARRLFQSQRTLGNPHASSNLEEAFLHLVAFQNPFASGEDIRNKAGHCSLEPDQIRA
PRRSASAETFMLLQKTGNLRLIHRRTGEERPLTDKEREQIHLLAWKQEKVTHKTLRRHLEIPEE
WLFTGLPYHRSGDKAEEKLFVHLAGIHEIRKALDKGPDPAVWDTLRSRRDLLDSIADTLTFYKN
EDEILPRLESLGLSPENARALAPLSFSGTAHLSLSALGKLLPHLEEGKSYTQARADAGYAAPPP
DRHPKLPPLEEADWRNPVVFRALTQTRKVVNALVRRYGPPWCIHLETARELSQPAKVRRRIETE
QQANEKKKQQAEREFLDIVGTAPGPGDLLKMRLWREQGGFCPYCEEYLNPTRLAEPGYAEMDHI
LPYSRSLDNGWHNRVLVHGKDNRDKGNRTPFEAFGGDTARWDRLVAWVQASHLSAPKKRNLLRE
DFGEEAERELKDRNLTDTRFITKTAATLLRDRLTFHPEAPKDPVMTLNGRLTAFLRKQWGLHKN
RKNGDLHHALDAAVLAVASRSFVYRLSSHNAAWGELPRGREAENGFSLPYPAFRSEVLARLCPT
REEILLRLDQGGVGYDEAFRNGLRPVFVSRAPSRRLRGKAHMETLRSPKWKDHPEGPRTASRIP
LKDLNLEKLERMVGKDRDRKLYEALRERLAAFGGNGKKAFVAPFRKPCRSGEGPLVRSLRIFDS
GYSGVELRDGGEVYAVADHESMVRVDVYAKKNRFYLVPVYVADVARGIVKNRAIVAHKSEEEWD
LVDGSFDFRFSLFPGDLVEIEKKDGAYLGYYKSCHRGDGRLLLDRHDRMPRESDCGTFYVSTRK
DVLSMSKYQVDPLGEIRLVGSEKPPFVL
SEQ ID NO: 358
MEKKRKVTLGFDLGIASVGWAIVDSETNQVYKLGSRLFDAPDTNLERRTQRGTRRLLRRRKYRN
QKFYNLVKRTEVFGLSSREAIENRFRELSIKYPNIIELKTKALSQEVCPDEIAWILHDYLKNRG
YFYDEKETKEDFDQQTVESMPSYKLNEFYKKYGYFKGALSQPTESEMKDNKDLKEAFFFDFSNK
EWLKEINYFFNVQKNILSETFIEEFKKIFSFTRDISKGPGSDNMPSPYGIFGEFGDNGQGGRYE
HIWDKNIGKCSIFTNEQRAPKYLPSALIFNFLNELANIRLYSTDKKNIQPLWKLSSVDKLNILL
NLFNLPISEKKKKLTSTNINDIVKKESIKSIMISVEDIDMIKDEWAGKEPNVYGVGLSGLNIEE
SAKENKFKFQDLKILNVLINLLDNVGIKFEFKDRNDIIKNLELLDNLYLFLIYQKESNNKDSSI
DLFIAKNESLNIENLKLKLKEFLLGAGNEFENHNSKTHSLSKKAIDEILPKLLDNNEGWNLEAI
KNYDEEIKSQIEDNSSLMAKQDKKYLNDNFLKDAILPPNVKVTFQQAILIFNKIIQKFSKDFEI
DKVVIELAREMTQDQENDALKGIAKAQKSKKSLVEERLEANNIDKSVFNDKYEKLIYKIFLWIS
QDFKDPYTGAQISVNEIVNNKVEIDHIIPYSLCFDDSSANKVLVHKQSNQEKSNSLPYEYIKQG
HSGWNWDEFTKYVKRVFVNNVDSILSKKERLKKSENLLTASYDGYDKLGFLARNLNDTRYATIL
FRDQLNNYAEHHLIDNKKMFKVIAMNGAVTSFIRKNMSYDNKLRLKDRSDFSHHAYDAAIIALF
SNKTKTLYNLIDPSLNGIISKRSEGYWVIEDRYTGEIKELKKEDWTSIKNNVQARKIAKEIEEY
LIDLDDEVFFSRKTKRKTNRQLYNETIYGIATKTDEDGITNYYKKEKFSILDDKDIYLRLLRER
EKFVINQSNPEVIDQIIEIIESYGKENNIPSRDEAINIKYTKNKINYNLYLKQYMRSLTKSLDQ
FSEEFINQMIANKTFVLYNPTKNTTRKIKFLRLVNDVKINDIRKNQVINKFNGKNNEPKAFYEN
INSLGAIVFKNSANNFKTLSINTQIAIFGDKNWDIEDFKTYNMEKIEKYKEIYGIDKTYNFHSF
IFPGTILLDKQNKEFYYISSIQTVRDIIEIKFLNKIEFKDENKNQDTSKTPKRLMFGIKSIMNN
YEQVDISPFGINKKIFE
SEQ ID NO: 359
MGYRIGLDVGITSTGYAVLKTDKNGLPYKILTLDSVIYPRAENPQTGASLAEPRRIKRGLRRRT
RRTKFRKQRTQQLFIHSGLLSKPEIEQILATPQAKYSVYELRVAGLDRRLTNSELFRVLYFFIG
HRGFKSNRKAELNPENEADKKQMGQLLNSIEEIRKAIAEKGYRTVGELYLKDPKYNDHKRNKGY
IDGYLSTPNRQMLVDEIKQILDKQRELGNEKLTDEFYATYLLGDENRAGIFQAQRDFDEGPGAG
PYAGDQIKKMVGKDIFEPTEDRAAKATYTFQYFNLLQKMTSLNYQNTTGDTWHTLNGLDRQAII
DAVFAKAEKPTKTYKPTDFGELRKLLKLPDDARFNLVNYGSLQTQKEIETVEKKTRFVDFKAYH
DLVKVLPEEMWQSRQLLDHIGTALTLYSSDKRRRRYFAEELNLPAELIEKLLPLNFSKFGHLSI
KSMQNIIPYLEMGQVYSEATTNTGYDFRKKQISKDTIREEITNPVVRRAVTKTIKIVEQIIRRY
GKPDGINIELARELGRNFKERGDIQKRQDKNRQTNDKIAAELTELGIPVNGQNIIRYKLHKEQN
GVDPYTGDQIPFERAFSEGYEVDHIIPYSISWDDSYTNKVLTSAKCNREKGNRIPMVYLANNEQ
RLNALTNIADNIIRNSRKRQKLLKQKLSDEELKDWKQRNINDTRFITRVLYNYFRQAIEFNPEL
EKKQRVLPLNGEVTSKIRSRWGFLKVREDGDLHHAIDATVIAAITPKFIQQVTKYSQHQEVKNN
QALWHDAEIKDAEYAAEAQRMDADLFNKIFNGFPLPWPEFLDELLARISDNPVEMMKSRSWNTY
TPIEIAKLKPVFVVRLANHKISGPAHLDTIRSAKLFDEKGIVLSRVSITKLKINKKGQVATGDG
IYDPENSNNGDKVVYSAIRQALEAHNGSGELAFPDGYLEYVDHGTKKLVRKVRVAKKVSLPVRL
KNKAAADNGSMVRIDVFNTGKKFVFVPIYIKDTVEQVLPNKAIARGKSLWYQITESDQFCFSLY
PGDMVHIESKTGIKPKYSNKENNTSVVPIKNFYGYFDGADIATASILVRAHDSSYTARSIGIAG
LLKFEKYQVDYFGRYHKVHEKKRQLFVKRDE
SEQ ID NO: 360
MQKNINTKQNHIYIKQAQKIKEKLGDKPYRIGLDLGVGSIGFAIVSMEENDGNVLLPKEIIMVG
SRIFKASAGAADRKLSRGQRNNHRHTRERMRYLWKVLAEQKLALPVPADLDRKENSSEGETSAK
RFLGDVLQKDIYELRVKSLDERLSLQELGYVLYHIAGHRGSSAIRTFENDSEEAQKENTENKKI
AGNIKRLMAKKNYRTYGEYLYKEFFENKEKHKREKISNAANNHKFSPTRDLVIKEAEAILKKQA
GKDGFHKELTEEYIEKLTKAIGYESEKLIPESGFCPYLKDEKRLPASHKLNEERRLWETLNNAR
YSDPIVDIVTGEITGYYEKQFTKEQKQKLFDYLLTGSELTPAQTKKLLGLKNTNFEDIILQGRD
KKAQKIKGYKLIKLESMPFWARLSEAQQDSFLYDWNSCPDEKLLTEKLSNEYHLTEEEIDNAFN
EIVLSSSYAPLGKSAMLIILEKIKNDLSYTEAVEEALKEGKLTKEKQAIKDRLPYYGAVLQEST
QKIIAKGFSPQFKDKGYKTPHTNKYELEYGRIANPVVHQTLNELRKLVNEIIDILGKKPCEIGL
ETARELKKSAEDRSKLSREQNDNESNRNRIYEIYIRPQQQVIITRRENPRNYILKFELLEEQKS
QCPFCGGQISPNDIINNQADIEHLFPIAESEDNGRNNLVISHSACNADKAKRSPWAAFASAAKD
SKYDYNRILSNVKENIPHKAWRFNQGAFEKFIENKPMAARFKTDNSYISKVAHKYLACLFEKPN
IICVKGSLTAQLRMAWGLQGLMIPFAKQLITEKESESFNKDVNSNKKIRLDNRHHALDAIVIAY
ASRGYGNLLNKMAGKDYKINYSERNWLSKILLPPNNIVWENIDADLESFESSVKTALKNAFISV
KHDHSDNGELVKGTMYKIFYSERGYTLTTYKKLSALKLTDPQKKKTPKDFLETALLKFKGRESE
MKNEKIKSAIENNKRLFDVIQDNLEKAKKLLEEENEKSKAEGKKEKNINDASIYQKAISLSGDK
YVQLSKKEPGKFFAISKPTPTTTGYGYDTGDSLCVDLYYDNKGKLCGEIIRKIDAQQKNPLKYK
EQGFTLFERIYGGDILEVDFDIHSDKNSFRNNTGSAPENRVFIKVGTFTEITNNNIQIWFGNII
KSTGGQDDSFTINSMQQYNPRKLILSSCGFIKYRSPILKNKEG
SEQ ID NO: 361
MAAFKPNPINYILGLDIGIASVGWAMVEIDEDENPICLIDLGVRVFERAEVPKTGDSLAMARRL
ARSVRRLTRRRAHRLLRARRLLKREGVLQAADFDENGLIKSLPNTPWQLRAAALDRKLTPLEWS
AVLLHLIKHRGYLSQRKNEGETADKELGALLKGVADNAHALQTGDFRTPAELALNKFEKESGHI
RNQRGDYSHTFSRKDLQAELILLFEKQKEFGNPHVSGGLKEGIETLLMTQRPALSGDAVQKMLG
HCTFEPAEPKAAKNTYTAERFIWLTKLNNLRILEQGSERPLTDTERATLMDEPYRKSKLTYAQA
RKLLGLEDTAFFKGLRYGKDNAEASTLMEMKAYHAISRALEKEGLKDKKSPLNLSPELQDEIGT
AFSLFKTDEDITGRLKDRIQPEILEALLKHISFDKFVQISLKALRRIVPLMEQGKRYDEACAEI
YGDHYGKKNTEEKIYLPPIPADEIRNPVVLRALSQARKVINGVVRRYGSPARIHIETAREVGKS
FKDRKEIEKRQEENRKDREKAAAKFREYFPNFVGEPKSKDILKLRLYEQQHGKCLYSGKEINLG
RLNEKGYVEIDHALPFSRTWDDSFNNKVLVLGSENQNKGNQTPYEYFNGKDNSREWQEFKARVE
TSRFPRSKKQRILLQKFDEDGFKERNLNDTRYVNRFLCQFVADRMRLTGKGKKRVFASNGQITN
LLRGFWGLRKVRAENDRHHALDAVVVACSTVAMQQKITRFVRYKEMNAFDGKTIDKETGEVLHQ
KTHFPQPWEFFAQEVMIRVFGKPDGKPEFEEADTPEKLRTLLAEKLSSRPEAVHEYVTPLFVSR
APNRKMSGQGHMETVKSAKRLDEGVSVLRVPLTQLKLKDLEKMVNREREPKLYEALKARLEAHK
DDPAKAFAEPFYKYDKAGNRTQQVKAVRVEQVQKTGVWVRNHNGIADNATMVRVDVFEKGDKYY
LVPIYSWQVAKGILPDRAVVQGKDEEDWQLIDDSFNFKFSLHPNDLVEVITKKARMFGYFASCH
RGTGNINIRIHDLDHKIGKNGILEGIGVKTALSFQKYQIDELGKEIRPCRLKKRPPVR
SEQ ID NO: 362
MQTTNLSYILGLDLGIASVGWAVVEINENEDPIGLIDVGVRIFERAEVPKTGESLALSRRLARS
TRRLIRRRAHRLLLAKRFLKREGILSTIDLEKGLPNQAWELRVAGLERRLSAIEWGAVLLHLIK
HRGYLSKRKNESQTNNKELGALLSGVAQNHQLLQSDDYRTPAELALKKFAKEEGHIRNQRGAYT
HTFNRLDLLAELNLLFAQQHQFGNPHCKEHIQQYMTELLMWQKPALSGEAILKMLGKCTHEKNE
FKAAKHTYSAERFVWLTKLNNLRILEDGAERALNEEERQLLINHPYEKSKLTYAQVRKLLGLSE
QAIFKHLRYSKENAESATFMELKAWHAIRKALENQGLKDTWQDLAKKPDLLDEIGTAFSLYKTD
EDIQQYLTNKVPNSVINALLVSLNFDKFIELSLKSLRKILPLMEQGKRYDQACREIYGHHYGEA
NQKTSQLLPAIPAQEIRNPVVLRTLSQARKVINAIIRQYGSPARVHIETGRELGKSFKERREIQ
KQQEDNRTKRESAVQKFKELFSDFSSEPKSKDILKFRLYEQQHGKCLYSGKEINIHRLNEKGYV
EIDHALPFSRTWDDSFNNKVLVLASENQNKGNQTPYEWLQGKINSERWKNFVALVLGSQCSAAK
KQRLLTQVIDDNKFIDRNLNDTRYIARFLSNYIQENLLLVGKNKKNVFTPNGQITALLRSRWGL
IKARENNNRHHALDAIVVACATPSMQQKITRFIRFKEVHPYKIENRYEMVDQESGEIISPHFPE
PWAYFRQEVNIRVFDNHPDTVLKEMLPDRPQANHQFVQPLFVSRAPTRKMSGQGHMETIKSAKR
LAEGISVLRIPLTQLKPNLLENMVNKEREPALYAGLKARLAEFNQDPAKAFATPFYKQGGQQVK
AIRVEQVQKSGVLVRENNGVADNASIVRTDVFIKNNKFFLVPIYTWQVAKGILPNKAIVAHKNE
DEWEEMDEGAKFKFSLFPNDLVELKTKKEYFFGYYIGLDRATGNISLKEHDGEISKGKDGVYRV
GVKLALSFEKYQVDELGKNRQICRPQQRQPVR
SEQ ID NO: 363
MGIRFAFDLGTNSIGWAVWRTGPGVFGEDTAASLDGSGVLIFKDGRNPKDGQSLATMRRVPRQS
RKRRDRFVLRRRDLLAALRKAGLFPVDVEEGRRLAATDPYHLRAKALDESLTPHEMGRVIFHLN
QRRGFRSNRKADRQDREKGKIAEGSKRLAETLAATNCRTLGEFLWSRHRGTPRTRSPTRIRMEG
EGAKALYAFYPTREMVRAEFERLWTAQSRFAPDLLTPERHEEIAGILFRQRDLAPPKIGCCTFE
PSERRLPRALPSVEARGIYERLAHLRITTGPVSDRGLTRPERDVLASALLAGKSLTFKAVRKTL
KILPHALVNFEEAGEKGLDGALTAKLLSKPDHYGAAWHGLSFAEKDTFVGKLLDEADEERLIRR
LVTENRLSEDAARRCASIPLADGYGRLGRTANTEILAALVEETDETGTVVTYAEAVRRAGERTG
RNWHHSDERDGVILDRLPYYGEILQRHVVPGSGEPEEKNEAARWGRLANPTVHIGLNQLRKVVN
RLIAAHGRPDQIVVELARELKLNREQKERLDRENRKNREENERRTAILAEHGQRDTAENKIRLR
LFEEQARANAGIALCPYTGRAIGIAELFTSEVEIDHILPVSLTLDDSLANRVLCRREANREKRR
QTPFQAFGATPAWNDIVARAAKLPPNKRWRFDPAALERFEREGGFLGRQLNETKYLSRLAKIYL
GKICDPDRVYVTPGTLTGLLRARWGLNSILSDSNFKNRSDHRHHAVDAVVIGVLTRGMIQRIAH
DAARAEDQDLDRVFRDVPVPFEDFRDHVRERVSTITVAVKPEHGKGGALHEDTSYGLVPDTDPN
AALGNLVVRKPIRSLTAGEVDRVRDRALRARLGALAAPFRDESGRVRDAKGLAQALEAFGAENG
IRRVRILKPDASVVTIADRRTGVPYRAVAPGENHHVDIVQMRDGSWRGFAASVFEVNRPGWRPE
WEVKKLGGKLVMRLHKGDMVELSDKDGQRRVKVVQQIEISANRVRLSPHNDGGKLQDRHADADD
PFRWDLATIPLLKDRGCVAVRVDPIGVVTLRRSNV
SEQ ID NO: 364
MMEVFMGRLVLGLDIGITSVGFGIIDLDESEIVDYGVRLFKEGTAAENETRRTKRGGRRLKRRR
VTRREDMLHLLKQAGIISTSFHPLNNPYDVRVKGLNERLNGEELATALLHLCKHRGSSVETIED
DEAKAKEAGETKKVLSMNDQLLKSGKYVCEIQKERLRTNGHIRGHENNFKTRAYVDEAFQILSH
QDLSNELKSAIITIISRKRMYYDGPGGPLSPTPYGRYTYFGQKEPIDLIEKMRGKCSLFPNEPR
APKLAYSAELFNLLNDLNNLSIEGEKLTSEQKAMILKIVHEKGKITPKQLAKEVGVSLEQIRGF
RIDTKGSPLLSELTGYKMIREVLEKSNDEHLEDHVFYDEIAEILTKTKDIEGRKKQISELSSDL
NEESVHQLAGLTKFTAYHSLSFKALRLINEEMLKTELNQMQSITLFGLKQNNELSVKGMKNIQA
DDTAILSPVAKRAQRETFKVVNRLREIYGEFDSIVVEMAREKNSEEQRKAIRERQKFFEMRNKQ
VADIIGDDRKINAKLREKLVLYQEQDGKTAYSLEPIDLKLLIDDPNAYEVDHIIPISISLDDSI
TNKVLVTHRENQEKGNLTPISAFVKGRFTKGSLAQYKAYCLKLKEKNIKTNKGYRKKVEQYLLN
ENDIYKYDIQKEFINRNLVDTSYASRVVLNTLTTYFKQNEIPTKVFTVKGSLTNAFRRKINLKK
DRDEDYGHHAIDALIIASMPKMRLLSTIFSRYKIEDIYDESTGEVFSSGDDSMYYDDRYFAFIA
SLKAIKVRKFSHKIDTKPNRSVADETIYSTRVIDGKEKVVKKYKDIYDPKFTALAEDILNNAYQ
EKYLMALHDPQTFDQIVKVVNYYFEEMSKSEKYFTKDKKGRIKISGMNPLSLYRDEHGMLKKYS
KKGDGPAITQMKYFDGVLGNHIDISAHYQVRDKKVVLQQISPYRTDFYYSKENGYKFVTIRYKD
VRWSEKKKKYVIDQQDYAMKKAEKKIDDTYEFQFSMHRDELIGITKAEGEALIYPDETWHNFNF
FFHAGETPEILKFTATNNDKSNKIEVKPIHCYCKMRLMPTISKKIVRIDKYATDVVGNLYKVKK
NTLKFEFD
SEQ ID NO: 365
MKKILGVDLGITSFGYAILQETGKDLYRCLDNSVVMRNNPYDEKSGESSQSIRSTQKSMRRLIE
KRKKRIRCVAQTMERYGILDYSETMKINDPKNNPIKNRWQLRAVDAWKRPLSPQELFAIFAHMA
KHRGYKSIATEDLIYELELELGLNDPEKESEKKADERRQVYNALRHLEELRKKYGGETIAQTIH
RAVEAGDLRSYRNHDDYEKMIRREDIEEEIEKVLLRQAELGALGLPEEQVSELIDELKACITDQ
EMPTIDESLFGKCTFYKDELAAPAYSYLYDLYRLYKKLADLNIDGYEVTQEDREKVIEWVEKKI
AQGKNLKKITHKDLRKILGLAPEQKIFGVEDERIVKGKKEPRTFVPFFFLADIAKFKELFASIQ
KHPDALQIFRELAEILQRSKTPQEALDRLRALMAGKGIDTDDRELLELFKNKRSGTRELSHRYI
LEALPLFLEGYDEKEVQRILGFDDREDYSRYPKSLRHLHLREGNLFEKEENPINNHAVKSLASW
ALGLIADLSWRYGPFDEIILETTRDALPEKIRKEIDKAMREREKALDKIIGKYKKEFPSIDKRL
ARKIQLWERQKGLDLYSGKVINLSQLLDGSADIEHIVPQSLGGLSTDYNTIVTLKSVNAAKGNR
LPGDWLAGNPDYRERIGMLSEKGLIDWKKRKNLLAQSLDEIYTENTHSKGIRATSYLEALVAQV
LKRYYPFPDPELRKNGIGVRMIPGKVTSKTRSLLGIKSKSRETNFHHAEDALILSTLTRGWQNR
LHRMLRDNYGKSEAELKELWKKYMPHIEGLTLADYIDEAFRRFMSKGEESLFYRDMFDTIRSIS
YWVDKKPLSASSHKETVYSSRHEVPTLRKNILEAFDSLNVIKDRHKLTTEEFMKRYDKEIRQKL
WLHRIGNTNDESYRAVEERATQIAQILTRYQLMDAQNDKEIDEKFQQALKELITSPIEVTGKLL
RKMRFVYDKLNAMQIDRGLVETDKNMLGIHISKGPNEKLIFRRMDVNNAHELQKERSGILCYLN
EMLFIFNKKGLIHYGCLRSYLEKGQGSKYIALFNPRFPANPKAQPSKFTSDSKIKQVGIGSATG
IIKAHLDLDGHVRSYEVFGTLPEGSIEWFKEESGYGRVEDDPHH
SEQ ID NO: 366
MRPIEPWILGLDIGTDSLGWAVFSCEEKGPPTAKELLGGGVRLFDSGRDAKDHTSRQAERGAFR
RARRQTRTWPWRRDRLIALFQAAGLTPPAAETRQIALALRREAVSRPLAPDALWAALLHLAHHR
GFRSNRIDKRERAAAKALAKAKPAKATAKATAPAKEADDEAGFWEGAEAALRQRMAASGAPTVG
ALLADDLDRGQPVRMRYNQSDRDGVVAPTRALIAEELAEIVARQSSAYPGLDWPAVTRLVLDQR
PLRSKGAGPCAFLPGEDRALRALPTVQDFIIRQTLANLRLPSTSADEPRPLTDEEHAKALALLS
TARFVEWPALRRALGLKRGVKFTAETERNGAKQAARGTAGNLTEAILAPLIPGWSGWDLDRKDR
VFSDLWAARQDRSALLALIGDPRGPTRVTEDETAEAVADAIQIVLPTGRASLSAKAARAIAQAM
APGIGYDEAVTLALGLHHSHRPRQERLARLPYYAAALPDVGLDGDPVGPPPAEDDGAAAEAYYG
RIGNISVHIALNETRKIVNALLHRHGPILRLVMVETTRELKAGADERKRMIAEQAERERENAEI
DVELRKSDRWMANARERRQRVRLARRQNNLCPYTSTPIGHADLLGDAYDIDHVIPLARGGRDSL
DNMVLCQSDANKTKGDKTPWEAFHDKPGWIAQRDDFLARLDPQTAKALAWRFADDAGERVARKS
AEDEDQGFLPRQLTDTGYIARVALRYLSLVTNEPNAVVATNGRLTGLLRLAWDITPGPAPRDLL
PTPRDALRDDTAARRFLDGLTPPPLAKAVEGAVQARLAALGRSRVADAGLADALGLTLASLGGG
GKNRADHRHHFIDAAMIAVTTRGLINQINQASGAGRILDLRKWPRTNFEPPYPTFRAEVMKQWD
HIHPSIRPAHRDGGSLHAATVFGVRNRPDARVLVQRKPVEKLFLDANAKPLPADKIAEIIDGFA
SPRMAKRFKALLARYQAAHPEVPPALAALAVARDPAFGPRGMTANTVIAGRSDGDGEDAGLITP
FRANPKAAVRTMGNAVYEVWEIQVKGRPRWTHRVLTRFDRTQPAPPPPPENARLVMRLRRGDLV
YWPLESGDRLFLVKKMAVDGRLALWPARLATGKATALYAQLSCPNINLNGDQGYCVQSAEGIRK
EKIRTTSCTALGRLRLSKKAT
SEQ ID NO: 367
MKYTLGLDVGIASVGWAVIDKDNNKIIDLGVRCFDKAEESKTGESLATARRIARGMRRRISRRS
QRLRLVKKLFVQYEIIKDSSEFNRIFDTSRDGWKDPWELRYNALSRILKPYELVQVLTHITKRR
GFKSNRKEDLSTTKEGVVITSIKNNSEMLRTKNYRTIGEMIFMETPENSNKRNKVDEYIHTIAR
EDLLNEIKYIFSIQRKLGSPFVTEKLEHDFLNIWEFQRPFASGDSILSKVGKCTLLKEELRAPT
SCYTSEYFGLLQSINNLVLVEDNNTLTLNNDQRAKIIEYAHFKNEIKYSEIRKLLDIEPEILFK
AHNLTHKNPSGNNESKKFYEMKSYHKLKSTLPTDIWGKLHSNKESLDNLFYCLTVYKNDNEIKD
YLQANNLDYLIEYIAKLPTFNKFKHLSLVAMKRIIPFMEKGYKYSDACNMAELDFTGSSKLEKC
NKLTVEPIIENVTNPVVIRALTQARKVINAIIQKYGLPYMVNIELAREAGMTRQDRDNLKKEHE
NNRKAREKISDLIRQNGRVASGLDILKWRLWEDQGGRCAYSGKPIPVCDLLNDSLTQIDHIYPY
SRSMDDSYMNKVLVLTDENQNKRSYTPYEVWGSTEKWEDFEARIYSMHLPQSKEKRLLNRNFIT
KDLDSFISRNLNDTRYISRFLKNYIESYLQFSNDSPKSCVVCVNGQCTAQLRSRWGLNKNREES
DLHHALDAAVIACADRKIIKEITNYYNERENHNYKVKYPLPWHSFRQDLMETLAGVFISRAPRR
KITGPAHDETIRSPKHFNKGLTSVKIPLTTVTLEKLETMVKNTKGGISDKAVYNVLKNRLIEHN
NKPLKAFAEKIYKPLKNGTNGAIIRSIRVETPSYTGVFRNEGKGISDNSLMVRVDVFKKKDKYY
LVPIYVAHMIKKELPSKAIVPLKPESQWELIDSTHEFLFSLYQNDYLVIKTKKGITEGYYRSCH
RGTGSLSLMPHFANNKNVKIDIGVRTAISIEKYNVDILGNKSIVKGEPRRGMEKYNSFKSN
SEQ ID NO: 368
MIRTLGIDIGIASIGWAVIEGEYTDKGLENKEIVASGVRVFTKAENPKNKESLALPRTLARSAR
RRNARKKGRIQQVKHYLSKALGLDLECFVQGEKLATLFQTSKDFLSPWELRERALYRVLDKEEL
ARVILHIAKRRGYDDITYGVEDNDSGKIKKAIAENSKRIKEEQCKTIGEMMYKLYFQKSLNVRN
KKESYNRCVGRSELREELKTIFQIQQELKSPWVNEELIYKLLGNPDAQSKQEREGLIFYQRPLK
GFGDKIGKCSHIKKGENSPYRACKHAPSAEEFVALTKSINFLKNLTNRHGLCFSQEDMCVYLGK
ILQEAQKNEKGLTYSKLKLLLDLPSDFEFLGLDYSGKNPEKAVFLSLPSTFKLNKITQDRKTQD
KIANILGANKDWEAILKELESLQLSKEQIQTIKDAKLNFSKHINLSLEALYHLLPLMREGKRYD
EGVEILQERGIFSKPQPKNRQLLPPLSELAKEESYFDIPNPVLRRALSEFRKVVNALLEKYGGF
HYFHIELTRDVCKAKSARMQLEKINKKNKSENDAASQLLEVLGLPNTYNNRLKCKLWKQQEEYC
LYSGEKITIDHLKDQRALQIDHAFPLSRSLDDSQSNKVLCLTSSNQEKSNKTPYEWLGSDEKKW
DMYVGRVYSSNFSPSKKRKLTQKNFKERNEEDFLARNLVDTGYIGRVTKEYIKHSLSFLPLPDG
KKEHIRIISGSMTSTMRSFWGVQEKNRDHHLHHAQDAIIIACIEPSMIQKYTTYLKDKETHRLK
SHQKAQILREGDHKLSLRWPMSNFKDKIQESIQNIIPSHHVSHKVTGELHQETVRTKEFYYQAF
GGEEGVKKALKFGKIREINQGIVDNGAMVRVDIFKSKDKGKFYAVPIYTYDFAIGKLPNKAIVQ
GKKNGIIKDWLEMDENYEFCFSLFKNDCIKIQTKEMQEAVLAIYKSTNSAKATIELEHLSKYAL
KNEDEEKMFTDTDKEKNKTMTRESCGIQGLKVFQKVKLSVLGEVLEHKPRNRQNIALKTTPKHV
SEQ ID NO: 369
MKYSIGLDIGIASVGWSVINKDKERIEDMGVRIFQKAENPKDGSSLASSRREKRGSRRRNRRKK
HRLDRIKNILCESGLVKKNEIEKIYKNAYLKSPWELRAKSLEAKISNKEIAQILLHIAKRRGFK
SFRKTDRNADDTGKLLSGIQENKKIMEEKGYLTIGDMVAKDPKFNTHVRNKAGSYLFSFSRKLL
EDEVRKIQAKQKELGNTHFTDDVLEKYIEVFNSQRNFDEGPSKPSPYYSEIGQIAKMIGNCTFE
SSEKRTAKNTWSGERFVFLQKLNNFRIVGLSGKRPLTEEERDIVEKEVYLKKEVRYEKLRKILY
LKEEERFGDLNYSKDEKQDKKTEKTKFISLIGNYTIKKLNLSEKLKSEIEEDKSKLDKIIEILT
FNKSDKTIESNLKKLELSREDIEILLSEEFSGTLNLSLKAIKKILPYLEKGLSYNEACEKADYD
YKNNGIKFKRGELLPVVDKDLIANPVVLRAISQTRKVVNAIIRKYGTPHTIHVEVARDLAKSYD
DRQTIIKENKKRELENEKTKKFISEEFGIKNVKGKLLLKYRLYQEQEGRCAYSRKELSLSEVIL
DESMTDIDHIIPYSRSMDDSYSNKVLVLSGENRKKSNLLPKEYFDRQGRDWDTFVLNVKAMKIH
PRKKSNLLKEKFTREDNKDWKSRALNDTRYISRFVANYLENALEYRDDSPKKRVFMIPGQLTAQ
LRARWRLNKVRENGDLHHALDAAVVAVTDQKAINNISNISRYKELKNCKDVIPSIEYHADEETG
EVYFEEVKDTRFPMPWSGFDLELQKRLESENPREEFYNLLSDKRYLGWFNYEEGFIEKLRPVFV
SRMPNRGVKGQAHQETIRSSKKISNQIAVSKKPLNSIKLKDLEKMQGRDTDRKLYEALKNRLEE
YDDKPEKAFAEPFYKPTNSGKRGPLVRGIKVEEKQNVGVYVNGGQASNGSMVRIDVFRKNGKFY
TVPIYVHQTLLKELPNRAINGKPYKDWDLIDGSFEFLYSFYPNDLIEIEFGKSKSIKNDNKLTK
TEIPEVNLSEVLGYYRGMDTSTGAATIDTQDGKIQMRIGIKTVKNIKKYQVDVLGNVYKVKREK
RQTF
SEQ ID NO: 370
MSKKVSRRYEEQAQEICQRLGSRPYSIGLDLGVGSIGVAVAAYDPIKKQPSDLVFVSSRIFIPS
TGAAERRQKRGQRNSLRHRANRLKFLWKLLAERNLMLSYSEQDVPDPARLRFEDAVVRANPYEL
RLKGLNEQLTLSELGYALYHIANHRGSSSVRTFLDEEKSSDDKKLEEQQAMTEQLAKEKGISTF
IEVLTAFNTNGLIGYRNSESVKSKGVPVPTRDIISNEIDVLLQTQKQFYQEILSDEYCDRIVSA
ILFENEKIVPEAGCCPYFPDEKKLPRCHFLNEERRLWEAINNARIKMPMQEGAAKRYQSASFSD
EQRHILFHIARSGTDITPKLVQKEFPALKTSIIVLQGKEKAIQKIAGFRFRRLEEKSFWKRLSE
EQKDDFFSAWTNTPDDKRLSKYLMKHLLLTENEVVDALKTVSLIGDYGPIGKTATQLLMKHLED
GLTYTEALERGMETGEFQELSVWEQQSLLPYYGQILTGSTQALMGKYWHSAFKEKRDSEGFFKP
NTNSDEEKYGRIANPVVHQTLNELRKLMNELITILGAKPQEITVELARELKVGAEKREDIIKQQ
TKQEKEAVLAYSKYCEPNNLDKRYIERFRLLEDQAFVCPYCLEHISVADIAAGRADVDHIFPRD
DTADNSYGNKVVAHRQCNDIKGKRTPYAAFSNTSAWGPIMHYLDETPGMWRKRRKFETNEEEYA
KYLQSKGFVSRFESDNSYIAKAAKEYLRCLFNPNNVTAVGSLKGMETSILRKAWNLQGIDDLLG
SRHWSKDADTSPTMRKNRDDNRHHGLDAIVALYCSRSLVQMINTMSEQGKRAVEIEAMIPIPGY
ASEPNLSFEAQRELFRKKILEFMDLHAFVSMKTDNDANGALLKDTVYSILGADTQGEDLVFVVK
KKIKDIGVKIGDYEEVASAIRGRITDKQPKWYPMEMKDKIEQLQSKNEAALQKYKESLVQAAAV
LEESNRKLIESGKKPIQLSEKTISKKALELVGGYYYLISNNKRTKTFVVKEPSNEVKGFAFDTG
SNLCLDFYHDAQGKLCGEIIRKIQAMNPSYKPAYMKQGYSLYVRLYQGDVCELRASDLTEAESN
LAKTTHVRLPNAKPGRTFVIIITFTEMGSGYQIYFSNLAKSKKGQDTSFTLTTIKNYDVRKVQL
SSAGLVRYVSPLLVDKIEKDEVALCGE
SEQ ID NO: 371
MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHKIDVIDSND
DVGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAYSAD
LFNALNDLNNLVIQRDGLSKLEYHEKYHIIENVFKQKKKPTLKQIANEINVNPEDIKGYRITKS
GKPQFTEFKLYHDLKSVLFDQSILENEDVLDQIAEILTIYQDKDSIKSKLTELDILLNEEDKEN
IAQLTGYTGTHRLSLKCIRLVLEEQWYSSRNQMEIFTHLNIKPKKINLTAANKIPKAMIDEFIL
SPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKVL
VKQSENSKKSNLTPYQYFNSGKSKLSYNQFKQHILNLSKSQDRISKKKKEYLLEERDINKFEVQ
KEFINRNLVDTRYATRELTNYLKAYFSANNMNVKVKTINGSFTDYLRKVWKFKKERNHGYKHHA
EDALIIANADFLFKENKKLKAVNSVLEKPEIESKQLDIQVDSEDNYSEMFIIPKQVQDIKDFRN
FKYSHRVDKKPNRQLINDTLYSTRKKDNSTYIVQTIKDIYAKDNTTLKKQFDKSPEKFLMYQHD
PRTFEKLEVIMKQYANEKNPLAKYHEETGEYLTKYSKKNNGPIVKSLKYIGNKLGSHLDVTHQF
KSSTKKLVKLSIKPYRFDVYLTDKGYKFITISYLDVLKKDNYYYIPEQKYDKLKLGKAIDKNAK
FIASFYKNDLIKLDGEIYKIIGVNSDTRNMIELDLPDIRYKEYCELNNIKGEPRIKKTIGKKVN
SIEKLTTDVLGNVFTNTQYTKPQLLFKRGN
SEQ ID NO: 372
MIMKLEKWRLGLDLGTNSIGWSVFSLDKDNSVQDLIDMGVRIFSDGRDPKTKEPLAVARRTARS
QRKLIYRRKLRRKQVFKFLQEQGLFPKTKEECMTLKSLNPYELRIKALDEKLEPYELGRALFNL
AVRRGFKSNRKDGSREEVSEKKSPDEIKTQADMQTHLEKAIKENGCRTITEFLYKNQGENGGIR
FAPGRMTYYPTRKMYEEEFNLIRSKQEKYYPQVDWDDIYKAIFYQRPLKPQQRGYCIYENDKER
TFKAMPCSQKLRILQDIGNLAYYEGGSKKRVELNDNQDKVLYELLNSKDKVTFDQMRKALCLAD
SNSFNLEENRDFLIGNPTAVKMRSKNRFGKLWDEIPLEEQDLIIETIITADEDDAVYEVIKKYD
LTQEQRDFIVKNTILQSGTSMLCKEVSEKLVKRLEEIADLKYHEAVESLGYKFADQTVEKYDLL
PYYGKVLPGSTMEIDLSAPETNPEKHYGKISNPTVHVALNQTRVVVNALIKEYGKPSQIAIELS
RDLKNNVEKKAEIARKQNQRAKENIAINDTISALYHTAFPGKSFYPNRNDRMKYRLWSELGLGN
KCIYCGKGISGAELFTKEIEIEHILPFSRTLLDAESNLTVAHSSCNAFKAERSPFEAFGTNPSG
YSWQEIIQRANQLKNTSKKNKFSPNAMDSFEKDSSFIARQLSDNQYIAKAALRYLKCLVENPSD
VWTTNGSMTKLLRDKWEMDSILCRKFTEKEVALLGLKPEQIGNYKKNRFDHRHHAIDAVVIGLT
DRSMVQKLATKNSHKGNRIEIPEFPILRSDLIEKVKNIVVSFKPDHGAEGKLSKETLLGKIKLH
GKETFVCRENIVSLSEKNLDDIVDEIKSKVKDYVAKHKGQKIEAVLSDFSKENGIKKVRCVNRV
QTPIEITSGKISRYLSPEDYFAAVIWEIPGEKKTFKAQYIRRNEVEKNSKGLNVVKPAVLENGK
PHPAAKQVCLLHKDDYLEFSDKGKMYFCRIAGYAATNNKLDIRPVYAVSYCADWINSTNETMLT
GYWKPTPTQNWVSVNVLFDKQKARLVTVSPIGRVFRK
SEQ ID NO: 373
MSSKAIDSLEQLDLFKPQEYTLGLDLGIKSIGWAILSGERIANAGVYLFETAEELNSTGNKLIS
KAAERGRKRRIRRMLDRKARRGRHIRYLLEREGLPTDELEEVVVHQSNRTLWDVRAEAVERKLT
KQELAAVLFHLVRHRGYFPNTKKLPPDDESDSADEEQGKINRATSRLREELKASDCKTIGQFLA
QNRDRQRNREGDYSNLMARKLVFEEALQILAFQRKQGHELSKDFEKTYLDVLMGQRSGRSPKLG
NCSLIPSELRAPSSAPSTEWFKFLQNLGNLQISNAYREEWSIDAPRRAQIIDACSQRSTSSYWQ
IRRDFQIPDEYRFNLVNYERRDPDVDLQEYLQQQERKTLANFRNWKQLEKIIGTGHPIQTLDEA
ARLITLIKDDEKLSDQLADLLPEASDKAITQLCELDFTTAAKISLEAMYRILPHMNQGMGFFDA
CQQESLPEIGVPPAGDRVPPFDEMYNPVVNRVLSQSRKLINAVIDEYGMPAKIRVELARDLGKG
RELRERIKLDQLDKSKQNDQRAEDFRAEFQQAPRGDQSLRYRLWKEQNCTCPYSGRMIPVNSVL
SEDTQIDHILPISQSFDNSLSNKVLCFTEENAQKSNRTPFEYLDAADFQRLEAISGNWPEAKRN
KLLHKSFGKVAEEWKSRALNDTRYLTSALADHLRHHLPDSKIQTVNGRITGYLRKQWGLEKDRD
KHTHHAVDAIVVACTTPAIVQQVTLYHQDIRRYKKLGEKRPTPWPETFRQDVLDVEEEIFITRQ
PKKVSGGIQTKDTLRKHRSKPDRQRVALTKVKLADLERLVEKDASNRNLYEHLKQCLEESGDQP
TKAFKAPFYMPSGPEAKQRPILSKVTLLREKPEPPKQLTELSGGRRYDSMAQGRLDIYRYKPGG
KRKDEYRVVLQRMIDLMRGEENVHVFQKGVPYDQGPEIEQNYTFLFSLYFDDLVEFQRSADSEV
IRGYYRTFNIANGQLKISTYLEGRQDFDFFGANRLAHFAKVQVNLLGKVIK
SEQ ID NO: 374
MRSLRYRLALDLGSTSLGWALFRLDACNRPTAVIKAGVRIFSDGRNPKDGSSLAVTRRAARAMR
RRRDRLLKRKTRMQAKLVEHGFFPADAGKRKALEQLNPYALRAKGLQEALLPGEFARALFHINQ
RRGFKSNRKTDKKDNDSGVLKKAIGQLRQQMAEQGSRTVGEYLWTRLQQGQGVRARYREKPYTT
EEGKKRIDKSYDLYIDRAMIEQEFDALWAAQAAFNPTLFHEAARADLKDTLLHQRPLRPVKPGR
CTLLPEEERAPLALPSTQRFRIHQEVNHLRLLDENLREVALTLAQRDAVVTALETKAKLSFEQI
RKLLKLSGSVQFNLEDAKRTELKGNATSAALARKELFGAAWSGFDEALQDEIVWQLVTEEGEGA
LIAWLQTHTGVDEARAQAIVDVSLPEGYGNLSRKALARIVPALRAAVITYDKAVQAAGFDHHSQ
LGFEYDASEVEDLVHPETGEIRSVFKQLPYYGKALQRHVAFGSGKPEDPDEKRYGKIANPTVHI
GLNQVRMVVNALIRRYGRPTEVVIELARDLKQSREQKVEAQRRQADNQRRNARIRRSIAEVLGI
GEERVRGSDIQKWICWEELSFDAADRRCPYSGVQISAAMLLSDEVEVEHILPFSKTLDDSLNNR
TVAMRQANRIKRNRTPWDARAEFEAQGWSYEDILQRAERMPLRKRYRFAPDGYERWLGDDKDFL
ARALNDTRYLSRVAAEYLRLVCPGTRVIPGQLTALLRGKFGLNDVLGLDGEKNRNDHRHHAVDA
CVIGVTDQGLMQRFATASAQARGDGLTRLVDGMPMPWPTYRDHVERAVRHIWVSHRPDHGFEGA
MMEETSYGIRKDGSIKQRRKADGSAGREISNLIRIHEATQPLRHGVSADGQPLAYKGYVGGSNY
CIEITVNDKGKWEGEVISTFRAYGVVRAGGMGRLRNPHEGQNGRKLIMRLVIGDSVRLEVDGAE
RTMRIVKISGSNGQIFMAPIHEANVDARNTDKQDAFTYTSKYAGSLQKAKTRRVTISPIGEVRD
PGFKG
SEQ ID NO: 375
MARPAFRAPRREHVNGWTPDPHRISKPFFILVSWHLLSRVVIDSSSGCFPGTSRDHTDKFAEWE
CAVQPYRLSFDLGTNSIGWGLLNLDRQGKPREIRALGSRIFSDGRDPQDKASLAVARRLARQMR
RRRDRYLTRRTRLMGALVRFGLMPADPAARKRLEVAVDPYLARERATRERLEPFEIGRALFHLN
QRRGYKPVRTATKPDEEAGKVKEAVERLEAAIAAAGAPTLGAWFAWRKTRGETLRARLAGKGKE
AAYPFYPARRMLEAEFDTLWAEQARHHPDLLTAEAREILRHRIFHQRPLKPPPVGRCTLYPDDG
RAPRALPSAQRLRLFQELASLRVIHLDLSERPLTPAERDRIVAFVQGRPPKAGRKPGKVQKSVP
FEKLRGLLELPPGTGFSLESDKRPELLGDETGARIAPAFGPGWTALPLEEQDALVELLLTEAEP
ERAIAALTARWALDEATAAKLAGATLPDFHGRYGRRAVAELLPVLERETRGDPDGRVRPIRLDE
AVKLLRGGKDHSDFSREGALLDALPYYGAVLERHVAFGTGNPADPEEKRVGRVANPTVHIALNQ
LRHLVNAILARHGRPEEIVIELARDLKRSAEDRRREDKRQADNQKRNEERKRLILSLGERPTPR
NLLKLRLWEEQGPVENRRCPYSGETISMRMLLSEQVDIDHILPFSVSLDDSAANKVVCLREANR
IKRNRSPWEAFGHDSERWAGILARAEALPKNKRWRFAPDALEKLEGEGGLRARHLNDTRHLSRL
AVEYLRCVCPKVRVSPGRLTALLRRRWGIDAILAEADGPPPEVPAETLDPSPAEKNRADHRHHA
LDAVVIGCIDRSMVQRVQLAAASAEREAAAREDNIRRVLEGFKEEPWDGFRAELERRARTIVVS
HRPEHGIGGALHKETAYGPVDPPEEGFNLVVRKPIDGLSKDEINSVRDPRLRRALIDRLAIRRR
DANDPATALAKAAEDLAAQPASRGIRRVRVLKKESNPIRVEHGGNPSGPRSGGPFHKLLLAGEV
HHVDVALRADGRRWVGHWVTLFEAHGGRGADGAAAPPRLGDGERFLMRLHKGDCLKLEHKGRVR
VMQVVKLEPSSNSVVVVEPHQVKTDRSKHVKISCDQLRARGARRVTVDPLGRVRVHAPGARVGI
GGDAGRTAMEPAEDIS
SEQ ID NO: 376
MKRTSLRAYRLGVDLGANSLGWFVVWLDDHGQPEGLGPGGVRIFPDGRNPQSKQSNAAGRRLAR
SARRRRDRYLQRRGKLMGLLVKHGLMPADEPARKRLECLDPYGLRAKALDEVLPLHHVGRALFH
LNQRRGLFANRAIEQGDKDASAIKAAAGRLQTSMQACGARTLGEFLNRRHQLRATVRARSPVGG
DVQARYEFYPTRAMVDAEFEAIWAAQAPHHPTMTAEAHDTIREAIFSQRAMKRPSIGKCSLDPA
TSQDDVDGFRCAWSHPLAQRFRIWQDVRNLAVVETGPTSSRLGKEDQDKVARALLQTDQLSFDE
IRGLLGLPSDARFNLESDRRDHLKGDATGAILSARRHFGPAWHDRSLDRQIDIVALLESALDEA
AIIASLGTTHSLDEAAAQRALSALLPDGYCRLGLRAIKRVLPLMEAGRTYAEAASAAGYDHALL
PGGKLSPTGYLPYYGQWLQNDVVGSDDERDTNERRWGRLPNPTVHIGIGQLRRVVNELIRWHGP
PAEITVELTRDLKLSPRRLAELEREQAENQRKNDKRTSLLRKLGLPASTHNLLKLRLWDEQGDV
ASECPYTGEAIGLERLVSDDVDIDHLIPFSISWDDSAANKVVCMRYANREKGNRTPFEAFGHRQ
GRPYDWADIAERAARLPRGKRWRFGPGARAQFEELGDFQARLLNETSWLARVAKQYLAAVTHPH
RIHVLPGRLTALLRATWELNDLLPGSDDRAAKSRKDHRHHAIDALVAALTDQALLRRMANAHDD
TRRKIEVLLPWPTFRIDLETRLKAMLVSHKPDHGLQARLHEDTAYGTVEHPETEDGANLVYRKT
FVDISEKEIDRIRDRRLRDLVRAHVAGERQQGKTLKAAVLSFAQRRDIAGHPNGIRHVRLTKSI
KPDYLVPIRDKAGRIYKSYNAGENAFVDILQAESGRWIARATTVFQANQANESHDAPAAQPIMR
VFKGDMLRIDHAGAEKFVKIVRLSPSNNLLYLVEHHQAGVFQTRHDDPEDSFRWLFASFDKLRE
WNAELVRIDTLGQPWRRKRGLETGSEDATRIGWTRPKKWP
SEQ ID NO: 377
MERIFGFDIGTTSIGFSVIDYSSTQSAGNIQRLGVRIFPEARDPDGTPLNQQRRQKRMMRRQLR
RRRIRRKALNETLHEAGFLPAYGSADWPVVMADEPYELRRRGLEEGLSAYEFGRAIYHLAQHRH
FKGRELEESDTPDPDVDDEKEAANERAATLKALKNEQTTLGAWLARRPPSDRKRGIHAHRNVVA
EEFERLWEVQSKFHPALKSEEMRARISDTIFAQRPVFWRKNTLGECRFMPGEPLCPKGSWLSQQ
RRMLEKLNNLAIAGGNARPLDAEERDAILSKLQQQASMSWPGVRSALKALYKQRGEPGAEKSLK
FNLELGGESKLLGNALEAKLADMFGPDWPAHPRKQEIRHAVHERLWAADYGETPDKKRVIILSE
KDRKAHREAAANSFVADFGITGEQAAQLQALKLPTGWEPYSIPALNLFLAELEKGERFGALVNG
PDWEGWRRTNFPHRNQPTGEILDKLPSPASKEERERISQLRNPTVVRTQNELRKVVNNLIGLYG
KPDRIRIEVGRDVGKSKREREEIQSGIRRNEKQRKKATEDLIKNGIANPSRDDVEKWILWKEGQ
ERCPYTGDQIGFNALFREGRYEVEHIWPRSRSFDNSPRNKTLCRKDVNIEKGNRMPFEAFGHDE
DRWSAIQIRLQGMVSAKGGTGMSPGKVKRFLAKTMPEDFAARQLNDTRYAAKQILAQLKRLWPD
MGPEAPVKVEAVTGQVTAQLRKLWTLNNILADDGEKTRADHRHHAIDALTVACTHPGMTNKLSR
YWQLRDDPRAEKPALTPPWDTIRADAEKAVSEIVVSHRVRKKVSGPLHKETTYGDTGTDIKTKS
GTYRQFVTRKKIESLSKGELDEIRDPRIKEIVAAHVAGRGGDPKKAFPPYPCVSPGGPEIRKVR
LTSKQQLNLMAQTGNGYADLGSNHHIAIYRLPDGKADFEIVSLFDASRRLAQRNPIVQRTRADG
ASFVMSLAAGEAIMIPEGSKKGIWIVQGVWASGQVVLERDTDADHSTTTRPMPNPILKDDAKKV
SIDPIGRVRPSND
SEQ ID NO: 378
MNKRILGLDTGTNSLGWAVVDWDEHAQSYELIKYGDVIFQEGVKIEKGIESSKAAERSGYKAIR
KQYFRRRLRKIQVLKVLVKYHLCPYLSDDDLRQWHLQKQYPKSDELMLWQRTSDEEGKNPYYDR
HRCLHEKLDLTVEADRYTLGRALYHLTQRRGFLSNRLDTSADNKEDGVVKSGISQLSTEMEEAG
CEYLGDYFYKLYDAQGNKVRIRQRYTDRNKHYQHEFDAICEKQELSSELIEDLQRAIFFQLPLK
SQRHGVGRCTFERGKPRCADSHPDYEEFRMLCFVNNIQVKGPHDLELRPLTYEEREKIEPLFFR
KSKPNFDFEDIAKALAGKKNYAWIHDKEERAYKFNYRMTQGVPGCPTIAQLKSIFGDDWKTGIA
ETYTLIQKKNGSKSLQEMVDDVWNVLYSFSSVEKLKEFAHHKLQLDEESAEKFAKIKLSHSFAA
LSLKAIRKFLPFLRKGMYYTHASFFANIPTIVGKEIWNKEQNRKYIMENVGELVFNYQPKHREV
QGTIEMLIKDFLANNFELPAGATDKLYHPSMIETYPNAQRNEFGILQLGSPRTNAIRNPMAMRS
LHILRRVVNQLLKESIIDENTEVHVEYARELNDANKRRAIADRQKEQDKQHKKYGDEIRKLYKE
ETGKDIEPTQTDVLKFQLWEEQNHHCLYTGEQIGITDFIGSNPKFDIEHTIPQSVGGDSTQMNL
TLCDNRFNREVKKAKLPTELANHEEILTRIEPWKNKYEQLVKERDKQRTFAGMDKAVKDIRIQK
RHKLQMEIDYWRGKYERFTMTEVPEGFSRRQGTGIGLISRYAGLYLKSLFHQADSRNKSNVYVV
KGVATAEFRKMWGLQSEYEKKCRDNHSHHCMDAITIACIGKREYDLMAEYYRMEETFKQGRGSK
PKFSKPWATFTEDVLNIYKNLLVVHDTPNNMPKHTKKYVQTSIGKVLAQGDTARGSLHLDTYYG
AIERDGEIRYVVRRPLSSFTKPEELENIVDETVKRTIKEAIADKNFKQAIAEPIYMNEEKGILI
KKVRCFAKSVKQPINIRQHRDLSKKEYKQQYHVMNENNYLLAIYEGLVKNKVVREFEIVSYIEA
AKYYKRSQDRNIFSSIVPTHSTKYGLPLKTKLLMGQLVLMFEENPDEIQVDNTKDLVKRLYKVV
GIEKDGRIKFKYHQEARKEGLPIFSTPYKNNDDYAPIFRQSINNINILVDGIDFTIDILGKVTL
KE
SEQ ID NO: 379
MNYKMGLDIGIASVGWAVINLDLKRIEDLGVRIFDKAEHPQNGESLALPRRIARSARRRLRRRK
HRLERIRRLLVSENVLTKEEMNLLFKQKKQIDVWQLRVDALERKLNNDELARVLLHLAKRRGFK
SNRKSERNSKESSEFLKNIEENQSILAQYRSVGEMIVKDSKFAYHKRNKLDSYSNMIARDDLER
EIKLIFEKQREFNNPVCTERLEEKYLNIWSSQRPFASKEDIEKKVGFCTFEPKEKRAPKATYTF
QSFIVWEHINKLRLVSPDETRALTEIERNLLYKQAFSKNKMTYYDIRKLLNLSDDIHFKGLLYD
PKSSLKQIENIRFLELDSYHKIRKCIENVYGKDGIRMFNETDIDTFGYALTIFKDDEDIVAYLQ
NEYITKNGKRVSNLANKVYDKSLIDELLNLSFSKFAHLSMKAIRNILPYMEQGEIYSKACELAG
YNFTGPKKKEKALLLPVIPNIANPVVMRALTQSRKVVNAIIKKYGSPVSIHIELARDLSHSFDE
RKKIQKDQTENRKKNETAIKQLIEYELTKNPTGLDIVKFKLWSEQQGRCMYSLKPIELERLLEP
GYVEVDHILPYSRSLDDSYANKVLVLTKENREKGNHTPVEYLGLGSERWKKFEKFVLANKQFSK
KKKQNLLRLRYEETEEKEFKERNLNDTRYISKFFANFIKEHLKFADGDGGQKVYTINGKITAHL
RSRWDFNKNREESDLHHAVDAVIVACATQGMIKKITEFYKAREQNKESAKKKEPIFPQPWPHFA
DELKARLSKFPQESIEAFALGNYDRKKLESLRPVFVSRMPKRSVTGAAHQETLRRCVGIDEQSG
KIQTAVKTKLSDIKLDKDGHFPMYQKESDPRTYEAIRQRLLEHNNDPKKAFQEPLYKPKKNGEP
GPVIRTVKIIDTKNKVVHLDGSKTVAYNSNIVRTDVFEKDGKYYCVPVYTMDIMKGTLPNKAIE
ANKPYSEWKEMTEEYTFQFSLFPNDLVRIVLPREKTIKTSTNEEIIIKDIFAYYKTIDSATGGL
ELISHDRNFSLRGVGSKTLKRFEKYQVDVLGNIHKVKGEKRVGLAAPTNQKKGKTVDSLQSVSD
SEQ ID NO: 380
MRRLGLDLGTNSIGWCLLDLGDDGEPVSIFRTGARIFSDGRDPKSLGSLKATRREARLTRRRRD
RFIQRQKNLINALVKYGLMPADEIQRQALAYKDPYPIRKKALDEAIDPYEMGRAIFHINQRRGF
KSNRKSADNEAGVVKQSIADLEMKLGEAGARTIGEFLADRQATNDTVRARRLSGTNALYEFYPD
RYMLEQEFDTLWAKQAAFNPSLYIEAARERLKEIVFFQRKLKPQEVGRCIFLSDEDRISKALPS
FQRFRIYQELSNLAWIDHDGVAHRITASLALRDHLFDELEHKKKLTFKAMRAILRKQGVVDYPV
GFNLESDNRDHLIGNLTSCIMRDAKKMIGSAWDRLDEEEQDSFILMLQDDQKGDDEVRSILTQQ
YGLSDDVAEDCLDVRLPDGHGSLSKKAIDRILPVLRDQGLIYYDAVKEAGLGEANLYDPYAALS
DKLDYYGKALAGHVMGASGKFEDSDEKRYGTISNPTVHIALNQVRAVVNELIRLHGKPDEVVIE
IGRDLPMGADGKRELERFQKEGRAKNERARDELKKLGHIDSRESRQKFQLWEQLAKEPVDRCCP
FTGKMMSISDLFSDKVEIEHLLPFSLTLDDSMANKTVCFRQANRDKGNRAPFDAFGNSPAGYDW
QEILGRSQNLPYAKRWRFLPDAMKRFEADGGFLERQLNDTRYISRYTTEYISTIIPKNKIWVVT
GRLTSLLRGFWGLNSILRGHNTDDGTPAKKSRDDHRHHAIDAIVVGMTSRGLLQKVSKAARRSE
DLDLTRLFEGRIDPWDGFRDEVKKHIDAIIVSHRPRKKSQGALHNDTAYGIVEHAENGASTVVH
RVPITSLGKQSDIEKVRDPLIKSALLNETAGLSGKSFENAVQKWCADNSIKSLRIVETVSIIPI
TDKEGVAYKGYKGDGNAYMDIYQDPTSSKWKGEIVSRFDANQKGFIPSWQSQFPTARLIMRLRI
NDLLKLQDGEIEEIYRVQRLSGSKILMAPHTEANVDARDRDKNDTFKLTSKSPGKLQSASARKV
HISPTGLIREG
SEQ ID NO: 381
MKNILGLDLGLSSIGWSVIRENSEEQELVAMGSRVVSLTAAELSSFTQGNGVSINSQRTQKRTQ
RKGYDRYQLRRTLLRNKLDTLGMLPDDSLSYLPKLQLWGLRAKAVTQRIELNELGRVLLHLNQK
RGYKSIKSDFSGDKKITDYVKTVKTRYDELKEMRLTIGELFFRRLTENAFFRCKEQVYPRQAYV
EEFDCIMNCQRKFYPDILTDETIRCIRDEIIYYQRPLKSCKYLVSRCEFEKRFYLNAAGKKTEA
GPKVSPRTSPLFQVCRLWESINNIVVKDRRNEIVFISAEQRAALFDFLNTHEKLKGSDLLKLLG
LSKTYGYRLGEQFKTGIQGNKTRVEIERALGNYPDKKRLLQFNLQEESSSMVNTETGEIIPMIS
LSFEQEPLYRLWHVLYSIDDREQLQSVLRQKFGIDDDEVLERLSAIDLVKAGFGNKSSKAIRRI
LPFLQLGMNYAEACEAAGYNHSNNYTKAENEARALLDRLPAIKKNELRQPVVEKILNQMVNVVN
ALMEKYGRFDEIRVELARELKQSKEERSNTYKSINKNQRENEQIAKRIVEYGVPTRSRIQKYKM
WEESKHCCIYCGQPVDVGDFLRGFDVEVEHIIPKSLYFDDSFANKVCSCRSCNKEKNNRTAYDY
MKSKGEKALSDYVERVNTMYTNNQISKTKWQNLLTPVDKISIDFIDRQLRESQYIARKAKEILT
SICYNVTATSGSVTSFLRHVWGWDTVLHDLNFDRYKKVGLTEVIEVNHRGSVIRREQIKDWSKR
FDHRHHAIDALTIACTKQAYIQRLNNLRAEEGPDFNKMSLERYIQSQPHFSVAQVREAVDRILV
SFRAGKRAVTPGKRYIRKNRKRISVQSVLIPRGALSEESVYGVIHVWEKDEQGHVIQKQRAVMK
YPITSINREMLDKEKVVDKRIHRILSGRLAQYNDNPKEAFAKPVYIDKECRIPIRTVRCFAKPA
INTLVPLKKDDKGNPVAWVNPGNNHHVAIYRDEDGKYKERTVTFWEAVDRCRVGIPAIVTQPDT
IWDNILQRNDISENVLESLPDVKWQFVLSLQQNEMFILGMNEEDYRYAMDQQDYALLNKYLYRV
QKLSKSDYSFRYHTETSVEDKYDGKPNLKLSMQMGKLKRVSIKSLLGLNPHKVHISVLGEIKEI
S
SEQ ID NO: 382
MAEKQHRWGLDIGTNSIGWAVIALIEGRPAGLVATGSRIFSDGRNPKDGSSLAVERRGPRQMRR
RRDRYLRRRDRFMQALINVGLMPGDAAARKALVTENPYVLRQRGLDQALTLPEFGRALFHLNQR
RGFQSNRKTDRATAKESGKVKNAIAAFRAGMGNARTVGEALARRLEDGRPVRARMVGQGKDEHY
ELYIAREWIAQEFDALWASQQRFHAEVLADAARDRLRAILLFQRKLLPVPVGKCFLEPNQPRVA
AALPSAQRFRLMQELNHLRVMTLADKRERPLSFQERNDLLAQLVARPKCGFDMLRKIVFGANKE
AYRFTIESERRKELKGCDTAAKLAKVNALGTRWQALSLDEQDRLVCLLLDGENDAVLADALREH
YGLTDAQIDTLLGLSFEDGHMRLGRSALLRVLDALESGRDEQGLPLSYDKAVVAAGYPAHTADL
ENGERDALPYYGELLWRYTQDAPTAKNDAERKFGKIANPTVHIGLNQLRKLVNALIQRYGKPAQ
IVVELARNLKAGLEEKERIKKQQTANLERNERIRQKLQDAGVPDNRENRLRMRLFEELGQGNGL
GTPCIYSGRQISLQRLFSNDVQVDHILPFSKTLDDSFANKVLAQHDANRYKGNRGPFEAFGANR
DGYAWDDIRARAAVLPRNKRNRFAETAMQDWLHNETDFLARQLTDTAYLSRVARQYLTAICSKD
DVYVSPGRLTAMLRAKWGLNRVLDGVMEEQGRPAVKNRDDHRHHAIDAVVIGATDRAMLQQVAT
LAARAREQDAERLIGDMPTPWPNFLEDVRAAVARCVVSHKPDHGPEGGLHNDTAYGIVAGPFED
GRYRVRHRVSLFDLKPGDLSNVRCDAPLQAELEPIFEQDDARAREVALTALAERYRQRKVWLEE
LMSVLPIRPRGEDGKTLPDSAPYKAYKGDSNYCYELFINERGRWDGELISTFRANQAAYRRFRN
DPARFRRYTAGGRPLLMRLCINDYIAVGTAAERTIFRVVKMSENKITLAEHFEGGTLKQRDADK
DDPFKYLTKSPGALRDLGARRIFVDLIGRVLDPGIKGD
SEQ ID NO: 383
MIERILGVDLGISSLGWAIVEYDKDDEAANRIIDCGVRLFTAAETPKKKESPNKARREARGIRR
VLNRRRVRMNMIKKLFLRAGLIQDVDLDGEGGMFYSKANRADVWELRHDGLYRLLKGDELARVL
IHIAKHRGYKFIGDDEADEESGKVKKAGVVLRQNFEAAGCRTVGEWLWRERGANGKKRNKHGDY
EISIHRDLLVEEVEAIFVAQQEMRSTIATDALKAAYREIAFFVRPMQRIEKMVGHCTYFPEERR
APKSAPTAEKFIAISKFFSTVIIDNEGWEQKIIERKTLEELLDFAVSREKVEFRHLRKFLDLSD
NEIFKGLHYKGKPKTAKKREATLFDPNEPTELEFDKVEAEKKAWISLRGAAKLREALGNEFYGR
FVALGKHADEATKILTYYKDEGQKRRELTKLPLEAEMVERLVKIGFSDFLKLSLKAIRDILPAM
ESGARYDEAVLMLGVPHKEKSAILPPLNKTDIDILNPTVIRAFAQFRKVANALVRKYGAFDRVH
FELAREINTKGEIEDIKESQRKNEKERKEAADWIAETSFQVPLTRKNILKKRLYIQQDGRCAYT
GDVIELERLFDEGYCEIDHILPRSRSADDSFANKVLCLARANQQKTDRTPYEWFGHDAARWNAF
ETRTSAPSNRVRTGKGKIDRLLKKNFDENSEMAFKDRNLNDTRYMARAIKTYCEQYWVFKNSHT
KAPVQVRSGKLTSVLRYQWGLESKDRESHTHHAVDAIIIAFSTQGMVQKLSEYYRFKETHREKE
RPKLAVPLANFRDAVEEATRIENTETVKEGVEVKRLLISRPPRARVTGQAHEQTAKPYPRIKQV
KNKKKWRLAPIDEEKFESFKADRVASANQKNFYETSTIPRVDVYHKKGKFHLVPIYLHEMVLNE
LPNLSLGTNPEAMDENFFKFSIFKDDLISIQTQGTPKKPAKIIMGYFKNMHGANMVLSSINNSP
CEGFTCTPVSMDKKHKDKCKLCPEENRIAGRCLQGFLDYWSQEGLRPPRKEFECDQGVKFALDV
KKYQIDPLGYYYEVKQEKRLGTIPQMRSAKKLVKK
SEQ ID NO: 384
MNNSIKSKPEVTIGLDLGVGSVGWAIVDNETNIIHHLGSRLFSQAKTAEDRRSFRGVRRLIRRR
KYKLKRFVNLIWKYNSYFGFKNKEDILNNYQEQQKLHNTVLNLKSEALNAKIDPKALSWILHDY
LKNRGHFYEDNRDFNVYPTKELAKYFDKYGYYKGIIDSKEDNDNKLEEELTKYKFSNKHWLEEV
KKVLSNQTGLPEKFKEEYESLFSYVRNYSEGPGSINSVSPYGIYHLDEKEGKVVQKYNNIWDKT
IGKCNIFPDEYRAPKNSPIAMIFNEINELSTIRSYSIYLTGWFINQEFKKAYLNKLLDLLIKTN
GEKPIDARQFKKLREETIAESIGKETLKDVENEEKLEKEDHKWKLKGLKLNTNGKIQYNDLSSL
AKFVHKLKQHLKLDFLLEDQYATLDKINFLQSLFVYLGKHLRYSNRVDSANLKEFSDSNKLFER
ILQKQKDGLFKLFEQTDKDDEKILAQTHSLSTKAMLLAITRMTNLDNDEDNQKNNDKGWNFEAI
KNFDQKFIDITKKNNNLSLKQNKRYLDDRFINDAILSPGVKRILREATKVFNAILKQFSEEYDV
TKVVIELARELSEEKELENTKNYKKLIKKNGDKISEGLKALGISEDEIKDILKSPTKSYKFLLW
LQQDHIDPYSLKEIAFDDIFTKTEKFEIDHIIPYSISFDDSSSNKLLVLAESNQAKSNQTPYEF
ISSGNAGIKWEDYEAYCRKFKDGDSSLLDSTQRSKKFAKMMKTDTSSKYDIGFLARNLNDTRYA
TIVFRDALEDYANNHLVEDKPMFKVVCINGSVTSFLRKNFDDSSYAKKDRDKNIHHAVDASIIS
IFSNETKTLFNQLTQFADYKLFKNTDGSWKKIDPKTGVVTEVTDENWKQIRVRNQVSEIAKVIE
KYIQDSNIERKARYSRKIENKTNISLFNDTVYSAKKVGYEDQIKRKNLKTLDIHESAKENKNSK
VKRQFVYRKLVNVSLLNNDKLADLFAEKEDILMYRANPWVINLAEQIFNEYTENKKIKSQNVFE
KYMLDLTKEFPEKFSEFLVKSMLRNKTAIIYDDKKNIVHRIKRLKMLSSELKENKLSNVIIRSK
NQSGTKLSYQDTINSLALMIMRSIDPTAKKQYIRVPLNTLNLHLGDHDFDLHNMDAYLKKPKFV
KYLKANEIGDEYKPWRVLTSGTLLIHKKDKKLMYISSFQNLNDVIEIKNLIETEYKENDDSDSK
KKKKANRFLMTLSTILNDYILLDAKDNFDILGLSKNRIDEILNSKLGLDKIVK
SEQ ID NO: 385
MGGSEVGTVPVTWRLGVDVGERSIGLAAVSYEEDKPKEILAAVSWIHDGGVGDERSGASRLALR
GMARRARRLRRFRRARLRDLDMLLSELGWTPLPDKNVSPVDAWLARKRLAEEYVVDETERRRLL
GYAVSHMARHRGWRNPWTTIKDLKNLPQPSDSWERTRESLEARYSVSLEPGTVGQWAGYLLQRA
PGIRLNPTQQSAGRRAELSNATAFETRLRQEDVLWELRCIADVQGLPEDVVSNVIDAVFCQKRP
SVPAERIGRDPLDPSQLRASRACLEFQEYRIVAAVANLRIRDGSGSRPLSLEERNAVIEALLAQ
TERSLTWSDIALEILKLPNESDLTSVPEEDGPSSLAYSQFAPFDETSARIAEFIAKNRRKIPTF
AQWWQEQDRTSRSDLVAALADNSIAGEEEQELLVHLPDAELEALEGLALPSGRVAYSRLTLSGL
TRVMRDDGVDVHNARKTCFGVDDNWRPPLPALHEATGHPVVDRNLAILRKFLSSATMRWGPPQS
IVVELARGASESRERQAEEEAARRAHRKANDRIRAELRASGLSDPSPADLVRARLLELYDCHCM
YCGAPISWENSELDHIVPRTDGGSNRHENLAITCGACNKEKGRRPFASWAETSNRVQLRDVIDR
VQKLKYSGNMYWTRDEFSRYKKSVVARLKRRTSDPEVIQSIESTGYAAVALRDRLLSYGEKNGV
AQVAVFRGGVTAEARRWLDISIERLFSRVAIFAQSTSTKRLDRRHHAVDAVVLTTLTPGVAKTL
ADARSRRVSAEFWRRPSDVNRHSTEEPQSPAYRQWKESCSGLGDLLISTAARDSIAVAAPLRLR
PTGALHEETLRAFSEHTVGAAWKGAELRRIVEPEVYAAFLALTDPGGRFLKVSPSEDVLPADEN
RHIVLSDRVLGPRDRVKLFPDDRGSIRVRGGAAYIASFHHARVFRWGSSHSPSFALLRVSLADL
AVAGLLRDGVDVFTAELPPWTPAWRYASIALVKAVESGDAKQVGWLVPGDELDFGPEGVTTAAG
DLSMFLKYFPERHWVVTGFEDDKRINLKPAFLSAEQAEVLRTERSDRPDTLTEAGEILAQFFPR
CWRATVAKVLCHPGLTVIRRTALGQPRWRRGHLPYSWRPWSADPWSGGTP
SEQ ID NO: 386
MHNKKNITIGFDLGIASIGWAIIDSTTSKILDWGTRTFEERKTANERRAFRSTRRNIRRKAYRN
QRFINLILKYKDLFELKNISDIQRANKKDTENYEKIISFFTEIYKKCAAKHSNILEVKVKALDS
KIEKLDLIWILHDYLENRGFFYDLEEENVADKYEGIEHPSILLYDFFKKNGFFKSNSSIPKDLG
GYSFSNLQWVNEIKKLFEVQEINPEFSEKFLNLFTSVRDYAKGPGSEHSASEYGIFQKDEKGKV
FKKYDNIWDKTIGKCSFFVEENRSPVNYPSYEIFNLLNQLINLSTDLKTTNKKIWQLSSNDRNE
LLDELLKVKEKAKIISISLKKNEIKKIILKDFGFEKSDIDDQDTIEGRKIIKEEPTTKLEVTKH
LLATIYSHSSDSNWININNILEFLPYLDAICIILDREKSRGQDEVLKKLTEKNIFEVLKIDREK
QLDFVKSIFSNTKFNFKKIGNFSLKAIREFLPKMFEQNKNSEYLKWKDEEIRRKWEEQKSKLGK
TDKKTKYLNPRIFQDEIISPGTKNTFEQAVLVLNQIIKKYSKENIIDAIIIESPREKNDKKTIE
EIKKRNKKGKGKTLEKLFQILNLENKGYKLSDLETKPAKLLDRLRFYHQQDGIDLYTLDKINID
QLINGSQKYEIEHIIPYSMSYDNSQANKILTEKAENLKKGKLIASEYIKRNGDEFYNKYYEKAK
ELFINKYKKNKKLDSYVDLDEDSAKNRFRFLTLQDYDEFQVEFLARNLNDTRYSTKLFYHALVE
HFENNEFFTYIDENSSKHKVKISTIKGHVTKYFRAKPVQKNNGPNENLNNNKPEKIEKNRENNE
HHAVDAAIVAIIGNKNPQIANLLTLADNKTDKKFLLHDENYKENIETGELVKIPKFEVDKLAKV
EDLKKIIQEKYEEAKKHTAIKFSRKTRTILNGGLSDETLYGFKYDEKEDKYFKIIKKKLVTSKN
EELKKYFENPFGKKADGKSEYTVLMAQSHLSEFNKLKEIFEKYNGFSNKTGNAFVEYMNDLALK
EPTLKAEIESAKSVEKLLYYNFKPSDQFTYHDNINNKSFKRFYKNIRIIEYKSIPIKFKILSKH
DGGKSFKDTLFSLYSLVYKVYENGKESYKSIPVTSQMRNFGIDEFDFLDENLYNKEKLDIYKSD
FAKPIPVNCKPVFVLKKGSILKKKSLDIDDFKETKETEEGNYYFISTISKRFNRDTAYGLKPLK
LSVVKPVAEPSTNPIFKEYIPIHLDELGNEYPVKIKEHTDDEKLMCTIK

Nucleic Acids Encoding Cas9 Molecules

Nucleic acids encoding the Cas9 molecules or Cas9 polypeptides, e.g., an eaCas9 molecule or eaCas9 polypeptides are provided herein.

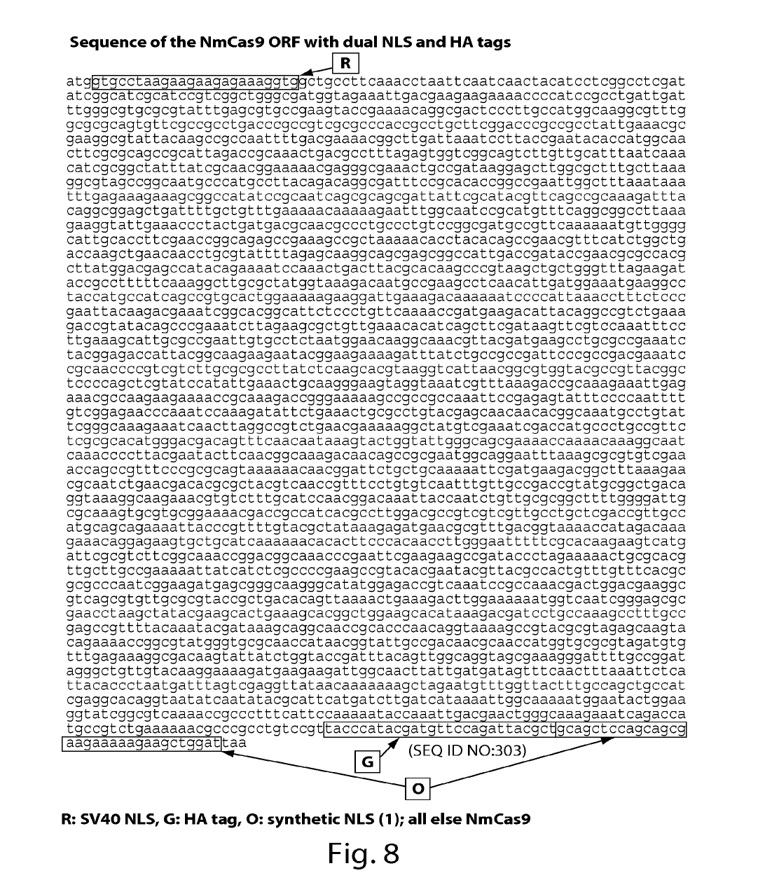

Exemplary nucleic acids encoding Cas9 molecules or Cas9 polypeptides are described in Cong et al., SCIENCE 2013, 399(6121):819-823; Wang et al., CELL 2013, 153(4):910-918; Mali et al., SCIENCE 2013, 399(6121):823-826; Jinek et al., SCIENCE 2012, 337(6096):816-821. Another exemplary nucleic acid encoding a Cas9 molecule or Cas9 polypeptide is shown in FIG. 8.

In an embodiment, a nucleic acid encoding a Cas9 molecule or Cas9 polypeptide can be a synthetic nucleic acid sequence. For example, the synthetic nucleic acid molecule can be chemically modified, e.g., as described in Section VIII. In an embodiment, the Cas9 mRNA has one or more (e.g., all of the following properties: it is capped, polyadenylated, substituted with 5-methylcytidine and/or pseudouridine.

In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule or Cas9 polypeptide may comprise a nuclear localization sequence (NLS). Nuclear localization sequences are known in the art.

Provided below is an exemplary codon optimized nucleic acid sequence encoding a Cas9 molecule of S. pyogenes.

(SEQ ID NO: 22)

ATGGATAAAA AGTACAGCAT CGGGCTGGAC ATCGGTACAA
ACTCAGTGGG GTGGGCCGTG ATTACGGACG AGTACAAGGT
ACCCTCCAAA AAATTTAAAG TGCTGGGTAA CACGGACAGA
CACTCTATAA AGAAAAATCT TATTGGAGCC TTGCTGTTCG
ACTCAGGCGA GACAGCCGAA GCCACAAGGT TGAAGCGGAC
CGCCAGGAGG CGGTATACCA GGAGAAAGAA CCGCATATGC
TACCTGCAAG AAATCTTCAG TAACGAGATG GCAAAGGTTG
ACGATAGCTT TTTCCATCGC CTGGAAGAAT CCTTTCTTGT
TGAGGAAGAC AAGAAGCACG AACGGCACCC CATCTTTGGC
AATATTGTCG ACGAAGTGGC ATATCACGAA AAGTACCCGA
CTATCTACCA CCTCAGGAAG AAGCTGGTGG ACTCTACCGA
TAAGGCGGAC CTCAGACTTA TTTATTTGGC ACTCGCCCAC
ATGATTAAAT TTAGAGGACA TTTCTTGATC GAGGGCGACC
TGAACCCGGA CAACAGTGAC GTCGATAAGC TGTTCATCCA
ACTTGTGCAG ACCTACAATC AACTGTTCGA AGAAAACCCT
ATAAATGCTT CAGGAGTCGA CGCTAAAGCA ATCCTGTCCG
CGCGCCTCTC AAAATCTAGA AGACTTGAGA ATCTGATTGC
TCAGTTGCCC GGGGAAAAGA AAAATGGATT GTTTGGCAAC
CTGATCGCCC TCAGTCTCGG ACTGACCCCA AATTTCAAAA
GTAACTTCGA CCTGGCCGAA GACGCTAAGC TCCAGCTGTC
CAAGGACACA TACGATGACG ACCTCGACAA TCTGCTGGCC
CAGATTGGGG ATCAGTACGC CGATCTCTTT TTGGCAGCAA
AGAACCTGTC CGACGCCATC CTGTTGAGCG ATATCTTGAG
AGTGAACACC GAAATTACTA AAGCACCCCT TAGCGCATCT
ATGATCAAGC GGTACGACGA GCATCATCAG GATCTGACCC
TGCTGAAGGC TCTTGTGAGG CAACAGCTCC CCGAAAAATA
CAAGGAAATC TTCTTTGACC AGAGCAAAAA CGGCTACGCT
GGCTATATAG ATGGTGGGGC CAGTCAGGAG GAATTCTATA
AATTCATCAA GCCCATTCTC GAGAAAATGG ACGGCACAGA
GGAGTTGCTG GTCAAACTTA ACAGGGAGGA CCTGCTGCGG
AAGCAGCGGA CCTTTGACAA CGGGTCTATC CCCCACCAGA
TTCATCTGGG CGAACTGCAC GCAATCCTGA GGAGGCAGGA
GGATTTTTAT CCTTTTCTTA AAGATAACCG CGAGAAAATA
GAAAAGATTC TTACATTCAG GATCCCGTAC TACGTGGGAC
CTCTCGCCCG GGGCAATTCA CGGTTTGCCT GGATGACAAG
GAAGTCAGAG GAGACTATTA CACCTTGGAA CTTCGAAGAA
GTGGTGGACA AGGGTGCATC TGCCCAGTCT TTCATCGAGC
GGATGACAAA TTTTGACAAG AACCTCCCTA ATGAGAAGGT
GCTGCCCAAA CATTCTCTGC TCTACGAGTA CTTTACCGTC
TACAATGAAC TGACTAAAGT CAAGTACGTC ACCGAGGGAA
TGAGGAAGCC GGCATTCCTT AGTGGAGAAC AGAAGAAGGC
GATTGTAGAC CTGTTGTTCA AGACCAACAG GAAGGTGACT
GTGAAGCAAC TTAAAGAAGA CTACTTTAAG AAGATCGAAT
GTTTTGACAG TGTGGAAATT TCAGGGGTTG AAGACCGCTT
CAATGCGTCA TTGGGGACTT ACCATGATCT TCTCAAGATC
ATAAAGGACA AAGACTTCCT GGACAACGAA GAAAATGAGG
ATATTCTCGA AGACATCGTC CTCACCCTGA CCCTGTTCGA
AGACAGGGAA ATGATAGAAG AGCGCTTGAA AACCTATGCC
CACCTCTTCG ACGATAAAGT TATGAAGCAG CTGAAGCGCA
GGAGATACAC AGGATGGGGA AGATTGTCAA GGAAGCTGAT
CAATGGAATT AGGGATAAAC AGAGTGGCAA GACCATACTG
GATTTCCTCA AATCTGATGG CTTCGCCAAT AGGAACTTCA
TGCAACTGAT TCACGATGAC TCTCTTACCT TCAAGGAGGA
CATTCAAAAG GCTCAGGTGA GCGGGCAGGG AGACTCCCTT
CATGAACACA TCGCGAATTT GGCAGGTTCC CCCGCTATTA
AAAAGGGCAT CCTTCAAACT GTCAAGGTGG TGGATGAATT
GGTCAAGGTA ATGGGCAGAC ATAAGCCAGA AAATATTGTG
ATCGAGATGG CCCGCGAAAA CCAGACCACA CAGAAGGGCC
AGAAAAATAG TAGAGAGCGG ATGAAGAGGA TCGAGGAGGG
CATCAAAGAG CTGGGATCTC AGATTCTCAA AGAACACCCC
GTAGAAAACA CACAGCTGCA GAACGAAAAA TTGTACTTGT
ACTATCTGCA GAACGGCAGA GACATGTACG TCGACCAAGA
ACTTGATATT AATAGACTGT CCGACTATGA CGTAGACCAT
ATCGTGCCCC AGTCCTTCCT GAAGGACGAC TCCATTGATA
ACAAAGTCTT GACAAGAAGC GACAAGAACA GGGGTAAAAG
TGATAATGTG CCTAGCGAGG AGGTGGTGAA AAAAATGAAG
AACTACTGGC GACAGCTGCT TAATGCAAAG CTCATTACAC
AACGGAAGTT CGATAATCTG ACGAAAGCAG AGAGAGGTGG
CTTGTCTGAG TTGGACAAGG CAGGGTTTAT TAAGCGGCAG
CTGGTGGAAA CTAGGCAGAT CACAAAGCAC GTGGCGCAGA
TTTTGGACAG CCGGATGAAC ACAAAATACG ACGAAAATGA
TAAACTGATA CGAGAGGTCA AAGTTATCAC GCTGAAAAGC
AAGCTGGTGT CCGATTTTCG GAAAGACTTC CAGTTCTACA
AAGTTCGCGA GATTAATAAC TACCATCATG CTCACGATGC
GTACCTGAAC GCTGTTGTCG GGACCGCCTT GATAAAGAAG
TACCCAAAGC TGGAATCCGA GTTCGTATAC GGGGATTACA
AAGTGTACGA TGTGAGGAAA ATGATAGCCA AGTCCGAGCA
GGAGATTGGA AAGGCCACAG CTAAGTACTT CTTTTATTCT
AACATCATGA ATTTTTTTAA GACGGAAATT ACCCTGGCCA
ACGGAGAGAT CAGAAAGCGG CCCCTTATAG AGACAAATGG
TGAAACAGGT GAAATCGTCT GGGATAAGGG CAGGGATTTC
GCTACTGTGA GGAAGGTGCT GAGTATGCCA CAGGTAAATA
TCGTGAAAAA AACCGAAGTA CAGACCGGAG GATTTTCCAA
GGAAAGCATT TTGCCTAAAA GAAACTCAGA CAAGCTCATC
GCCCGCAAGA AAGATTGGGA CCCTAAGAAA TACGGGGGAT
TTGACTCACC CACCGTAGCC TATTCTGTGC TGGTGGTAGC
TAAGGTGGAA AAAGGAAAGT CTAAGAAGCT GAAGTCCGTG
AAGGAACTCT TGGGAATCAC TATCATGGAA AGATCATCCT
TTGAAAAGAA CCCTATCGAT TTCCTGGAGG CTAAGGGTTA
CAAGGAGGTC AAGAAAGACC TCATCATTAA ACTGCCAAAA
TACTCTCTCT TCGAGCTGGA AAATGGCAGG AAGAGAATGT
TGGCCAGCGC CGGAGAGCTG CAAAAGGGAA ACGAGCTTGC
TCTGCCCTCC AAATATGTTA ATTTTCTCTA TCTCGCTTCC
CACTATGAAA AGCTGAAAGG GTCTCCCGAA GATAACGAGC
AGAAGCAGCT GTTCGTCGAA CAGCACAAGC ACTATCTGGA
TGAAATAATC GAACAAATAA GCGAGTTCAG CAAAAGGGTT
ATCCTGGCGG ATGCTAATTT GGACAAAGTA CTGTCTGCTT
ATAACAAGCA CCGGGATAAG CCTATTAGGG AACAAGCCGA
GAATATAATT CACCTCTTTA CACTCACGAA TCTCGGAGCC
CCCGCCGCCT TCAAATACTT TGATACGACT ATCGACCGGA
AACGGTATAC CAGTACCAAA GAGGTCCTCG ATGCCACCCT
CATCCACCAG TCAATTACTG GCCTGTACGA AACACGGATC
GACCTCTCTC AACTGGGCGG CGACTAG

Provided below is the corresponding amino acid sequence of a S. pyogenes Cas9 molecule.

(SEQ ID NO: 23)

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD*

Provided below is an exemplary codon optimized nucleic acid sequence encoding a Cas9 molecule of N. meningitides.

SEQ ID NO: 24)

ATGGCCGCCTTCAAGCCCAACCCCATCAACTACATCCTGGGCCTGGACAT
CGGCATCGCCAGCGTGGGCTGGGCCATGGTGGAGATCGACGAGGACGAGA
ACCCCATCTGCCTGATCGACCTGGGTGTGCGCGTGTTCGAGCGCGCTGAG
GTGCCCAAGACTGGTGACAGTCTGGCTATGGCTCGCCGGCTTGCTCGCTC
TGTTCGGCGCCTTACTCGCCGGCGCGCTCACCGCCTTCTGCGCGCTCGCC
GCCTGCTGAAGCGCGAGGGTGTGCTGCAGGCTGCCGACTTCGACGAGAAC
GGCCTGATCAAGAGCCTGCCCAACACTCCTTGGCAGCTGCGCGCTGCCGC
TCTGGACCGCAAGCTGACTCCTCTGGAGTGGAGCGCCGTGCTGCTGCACC
TGATCAAGCACCGCGGCTACCTGAGCCAGCGCAAGAACGAGGGCGAGACC
GCCGACAAGGAGCTGGGTGCTCTGCTGAAGGGCGTGGCCGACAACGCCCA
CGCCCTGCAGACTGGTGACTTCCGCACTCCTGCTGAGCTGGCCCTGAACA
AGTTCGAGAAGGAGAGCGGCCACATCCGCAACCAGCGCGGCGACTACAGC
CACACCTTCAGCCGCAAGGACCTGCAGGCCGAGCTGATCCTGCTGTTCGA
GAAGCAGAAGGAGTTCGGCAACCCCCACGTGAGCGGCGGCCTGAAGGAGG
GCATCGAGACCCTGCTGATGACCCAGCGCCCCGCCCTGAGCGGCGACGCC
GTGCAGAAGATGCTGGGCCACTGCACCTTCGAGCCAGCCGAGCCCAAGGC
CGCCAAGAACACCTACACCGCCGAGCGCTTCATCTGGCTGACCAAGCTGA
ACAACCTGCGCATCCTGGAGCAGGGCAGCGAGCGCCCCCTGACCGACACC
GAGCGCGCCACCCTGATGGACGAGCCCTACCGCAAGAGCAAGCTGACCTA
CGCCCAGGCCCGCAAGCTGCTGGGTCTGGAGGACACCGCCTTCTTCAAGG
GCCTGCGCTACGGCAAGGACAACGCCGAGGCCAGCACCCTGATGGAGATG
AAGGCCTACCACGCCATCAGCCGCGCCCTGGAGAAGGAGGGCCTGAAGGA
CAAGAAGAGTCCTCTGAACCTGAGCCCCGAGCTGCAGGACGAGATCGGCA
CCGCCTTCAGCCTGTTCAAGACCGACGAGGACATCACCGGCCGCCTGAAG
GACCGCATCCAGCCCGAGATCCTGGAGGCCCTGCTGAAGCACATCAGCTT
CGACAAGTTCGTGCAGATCAGCCTGAAGGCCCTGCGCCGCATCGTGCCCC
TGATGGAGCAGGGCAAGCGCTACGACGAGGCCTGCGCCGAGATCTACGGC
GACCACTACGGCAAGAAGAACACCGAGGAGAAGATCTACCTGCCTCCTAT
CCCCGCCGACGAGATCCGCAACCCCGTGGTGCTGCGCGCCCTGAGCCAGG
CCCGCAAGGTGATCAACGGCGTGGTGCGCCGCTACGGCAGCCCCGCCCGC
ATCCACATCGAGACCGCCCGCGAGGTGGGCAAGAGCTTCAAGGACCGCAA
GGAGATCGAGAAGCGCCAGGAGGAGAACCGCAAGGACCGCGAGAAGGCCG
CCGCCAAGTTCCGCGAGTACTTCCCCAACTTCGTGGGCGAGCCCAAGAGC
AAGGACATCCTGAAGCTGCGCCTGTACGAGCAGCAGCACGGCAAGTGCCT
GTACAGCGGCAAGGAGATCAACCTGGGCCGCCTGAACGAGAAGGGCTACG
TGGAGATCGACCACGCCCTGCCCTTCAGCCGCACCTGGGACGACAGCTTC
AACAACAAGGTGCTGGTGCTGGGCAGCGAGAACCAGAACAAGGGCAACCA
GACCCCCTACGAGTACTTCAACGGCAAGGACAACAGCCGCGAGTGGCAGG
AGTTCAAGGCCCGCGTGGAGACCAGCCGCTTCCCCCGCAGCAAGAAGCAG
CGCATCCTGCTGCAGAAGTTCGACGAGGACGGCTTCAAGGAGCGCAACCT
GAACGACACCCGCTACGTGAACCGCTTCCTGTGCCAGTTCGTGGCCGACC
GCATGCGCCTGACCGGCAAGGGCAAGAAGCGCGTGTTCGCCAGCAACGGC
CAGATCACCAACCTGCTGCGCGGCTTCTGGGGCCTGCGCAAGGTGCGCGC
CGAGAACGACCGCCACCACGCCCTGGACGCCGTGGTGGTGGCCTGCAGCA
CCGTGGCCATGCAGCAGAAGATCACCCGCTTCGTGCGCTACAAGGAGATG
AACGCCTTCGACGGTAAAACCATCGACAAGGAGACCGGCGAGGTGCTGCA
CCAGAAGACCCACTTCCCCCAGCCCTGGGAGTTCTTCGCCCAGGAGGTGA
TGATCCGCGTGTTCGGCAAGCCCGACGGCAAGCCCGAGTTCGAGGAGGCC
GACACCCCCGAGAAGCTGCGCACCCTGCTGGCCGAGAAGCTGAGCAGCCG
CCCTGAGGCCGTGCACGAGTACGTGACTCCTCTGTTCGTGAGCCGCGCCC
CCAACCGCAAGATGAGCGGTCAGGGTCACATGGAGACCGTGAAGAGCGCC
AAGCGCCTGGACGAGGGCGTGAGCGTGCTGCGCGTGCCCCTGACCCAGCT
GAAGCTGAAGGACCTGGAGAAGATGGTGAACCGCGAGCGCGAGCCCAAGC
TGTACGAGGCCCTGAAGGCCCGCCTGGAGGCCCACAAGGACGACCCCGCC
AAGGCCTTCGCCGAGCCCTTCTACAAGTACGACAAGGCCGGCAACCGCAC
CCAGCAGGTGAAGGCCGTGCGCGTGGAGCAGGTGCAGAAGACCGGCGTGT
GGGTGCGCAACCACAACGGCATCGCCGACAACGCCACCATGGTGCGCGTG
GACGTGTTCGAGAAGGGCGACAAGTACTACCTGGTGCCCATCTACAGCTG
GCAGGTGGCCAAGGGCATCCTGCCCGACCGCGCCGTGGTGCAGGGCAAGG
ACGAGGAGGACTGGCAGCTGATCGACGACAGCTTCAACTTCAAGTTCAGC
CTGCACCCCAACGACCTGGTGGAGGTGATCACCAAGAAGGCCCGCATGTT
CGGCTACTTCGCCAGCTGCCACCGCGGCACCGGCAACATCAACATCCGCA
TCCACGACCTGGACCACAAGATCGGCAAGAACGGCATCCTGGAGGGCATC
GGCGTGAAGACCGCCCTGAGCTTCCAGAAGTACCAGATCGACGAGCTGGG
CAAGGAGATCCGCCCCTGCCGCCTGAAGAAGCGCCCTCCTGTGCGCTAA

Provided below is the corresponding amino acid sequence of a N. meningitides Cas9 molecule.

(SEQ ID NO: 25)

MAAFKPNPINYILGLDIGIASVGWAMVEIDEDENPICLIDLGVRVFERAE
VPKTGDSLAMARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNAHALQTGDFRTPAELALNKFEKESGHIRNQRGDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSPELQDEIGTAFSLFKTDEDITGRLK
DRIQPEILEALLKHISFDKFVQISLKALRRIVPLMEQGKRYDEACAEIYG
DHYGKKNTEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQ
RILLQKFDEDGFKERNLNDTRYVNRFLCQFVADRMRLTGKGKKRVFASNG
QITNLLRGFWGLRKVRAENDRHHALDAVVVACSTVAMQQKITRFVRYKEM
NAFDGKTIDKETGEVLHQKTHFPQPWEFFAQEVMIRVFGKPDGKPEFEEA
DTPEKLRTLLAEKLSSRPEAVHEYVTPLFVSRAPNRKMSGQGHMETVKSA
KRLDEGVSVLRVPLTQLKLKDLEKMVNREREPKLYEALKARLEAHKDDPA
KAFAEPFYKYDKAGNRTQQVKAVRVEQVQKTGVWVRNHNGIADNATMVRV
DVFEKGDKYYLVPIYSWQVAKGILPDRAVVQGKDEEDWQLIDDSFNFKFS
LHPNDLVEVITKKARMFGYFASCHRGTGNINIRIHDLDHKIGKNGILEGI
GVKTALSFQKYQIDELGKEIRPCRLKKRPPVR*

Provided below is an amino acid sequence of a S. aureus Cas9 molecule.

(SEQ ID NO: 26)

MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG*

Provided below is an exemplary codon optimized nucleic acid sequence encoding a Cas9 molecule of S. aureus Cas9.

(SEQ ID NO: 39)

ATGAAAAGGAACTACATTCTGGGGCTGGACATCGGGATTACAAGCGTGGG
GTATGGGATTATTGACTATGAAACAAGGGACGTGATCGACGCAGGCGTCA
GACTGTTCAAGGAGGCCAACGTGGAAAACAATGAGGGACGGAGAAGCAAG
AGGGGAGCCAGGCGCCTGAAACGACGGAGAAGGCACAGAATCCAGAGGGT
GAAGAAACTGCTGTTCGATTACAACCTGCTGACCGACCATTCTGAGCTGA
GTGGAATTAATCCTTATGAAGCCAGGGTGAAAGGCCTGAGTCAGAAGCTG
TCAGAGGAAGAGTTTTCCGCAGCTCTGCTGCACCTGGCTAAGCGCCGAGG
AGTGCATAACGTCAATGAGGTGGAAGAGGACACCGGCAACGAGCTGTCTA
CAAAGGAACAGATCTCACGCAATAGCAAAGCTCTGGAAGAGAAGTATGTC
GCAGAGCTGCAGCTGGAACGGCTGAAGAAAGATGGCGAGGTGAGAGGGTC
AATTAATAGGTTCAAGACAAGCGACTACGTCAAAGAAGCCAAGCAGCTGC
TGAAAGTGCAGAAGGCTTACCACCAGCTGGATCAGAGCTTCATCGATACT
TATATCGACCTGCTGGAGACTCGGAGAACCTACTATGAGGGACCAGGAGA
AGGGAGCCCCTTCGGATGGAAAGACATCAAGGAATGGTACGAGATGCTGA
TGGGACATTGCACCTATTTTCCAGAAGAGCTGAGAAGCGTCAAGTACGCT
TATAACGCAGATCTGTACAACGCCCTGAATGACCTGAACAACCTGGTCAT
CACCAGGGATGAAAACGAGAAACTGGAATACTATGAGAAGTTCCAGATCA
TCGAAAACGTGTTTAAGCAGAAGAAAAAGCCTACACTGAAACAGATTGCT
AAGGAGATCCTGGTCAACGAAGAGGACATCAAGGGCTACCGGGTGACAAG
CACTGGAAAACCAGAGTTCACCAATCTGAAAGTGTATCACGATATTAAGG
ACATCACAGCACGGAAAGAAATCATTGAGAACGCCGAACTGCTGGATCAG
ATTGCTAAGATCCTGACTATCTACCAGAGCTCCGAGGACATCCAGGAAGA
GCTGACTAACCTGAACAGCGAGCTGACCCAGGAAGAGATCGAACAGATTA
GTAATCTGAAGGGGTACACCGGAACACACAACCTGTCCCTGAAAGCTATC
AATCTGATTCTGGATGAGCTGTGGCATACAAACGACAATCAGATTGCAAT
CTTTAACCGGCTGAAGCTGGTCCCAAAAAAGGTGGACCTGAGTCAGCAGA
AAGAGATCCCAACCACACTGGTGGACGATTTCATTCTGTCACCCGTGGTC
AAGCGGAGCTTCATCCAGAGCATCAAAGTGATCAACGCCATCATCAAGAA
GTACGGCCTGCCCAATGATATCATTATCGAGCTGGCTAGGGAGAAGAACA
GCAAGGACGCACAGAAGATGATCAATGAGATGCAGAAACGAAACCGGCAG
ACCAATGAACGCATTGAAGAGATTATCCGAACTACCGGGAAAGAGAACGC
AAAGTACCTGATTGAAAAAATCAAGCTGCACGATATGCAGGAGGGAAAGT
GTCTGTATTCTCTGGAGGCCATCCCCCTGGAGGACCTGCTGAACAATCCA
TTCAACTACGAGGTCGATCATATTATCCCCAGAAGCGTGTCCTTCGACAA
TTCCTTTAACAACAAGGTGCTGGTCAAGCAGGAAGAGAACTCTAAAAAGG
GCAATAGGACTCCTTTCCAGTACCTGTCTAGTTCAGATTCCAAGATCTCT
TACGAAACCTTTAAAAAGCACATTCTGAATCTGGCCAAAGGAAAGGGCCG
CATCAGCAAGACCAAAAAGGAGTACCTGCTGGAAGAGCGGGACATCAACA
GATTCTCCGTCCAGAAGGATTTTATTAACCGGAATCTGGTGGACACAAGA
TACGCTACTCGCGGCCTGATGAATCTGCTGCGATCCTATTTCCGGGTGAA
CAATCTGGATGTGAAAGTCAAGTCCATCAACGGCGGGTTCACATCTTTTC
TGAGGCGCAAATGGAAGTTTAAAAAGGAGCGCAACAAAGGGTACAAGCAC
CATGCCGAAGATGCTCTGATTATCGCAAATGCCGACTTCATCTTTAAGGA
GTGGAAAAAGCTGGACAAAGCCAAGAAAGTGATGGAGAACCAGATGTTCG
AAGAGAAGCAGGCCGAATCTATGCCCGAAATCGAGACAGAACAGGAGTAC
AAGGAGATTTTCATCACTCCTCACCAGATCAAGCATATCAAGGATTTCAA
GGACTACAAGTACTCTCACCGGGTGGATAAAAAGCCCAACAGAGAGCTGA
TCAATGACACCCTGTATAGTACAAGAAAAGACGATAAGGGGAATACCCTG
ATTGTGAACAATCTGAACGGACTGTACGACAAAGATAATGACAAGCTGAA
AAAGCTGATCAACAAAAGTCCCGAGAAGCTGCTGATGTACCACCATGATC
CTCAGACATATCAGAAACTGAAGCTGATTATGGAGCAGTACGGCGACGAG
AAGAACCCACTGTATAAGTACTATGAAGAGACTGGGAACTACCTGACCAA
GTATAGCAAAAAGGATAATGGCCCCGTGATCAAGAAGATCAAGTACTATG
GGAACAAGCTGAATGCCCATCTGGACATCACAGACGATTACCCTAACAGT
CGCAACAAGGTGGTCAAGCTGTCACTGAAGCCATACAGATTCGATGTCTA
TCTGGACAACGGCGTGTATAAATTTGTGACTGTCAAGAATCTGGATGTCA
TCAAAAAGGAGAACTACTATGAAGTGAATAGCAAGTGCTACGAAGAGGCT
AAAAAGCTGAAAAAGATTAGCAACCAGGCAGAGTTCATCGCCTCCTTTTA
CAACAACGACCTGATTAAGATCAATGGCGAACTGTATAGGGTCATCGGGG
TGAACAATGATCTGCTGAACCGCATTGAAGTGAATATGATTGACATCACT
TACCGAGAGTATCTGGAAAACATGAATGATAAGCGCCCCCCTCGAATTAT
CAAAACAATTGCCTCTAAGACTCAGAGTATCAAAAAGTACTCAACCGACA
TTCTGGGAAACCTGTATGAGGTGAAGAGCAAAAAGCACCCTCAGATTATC
AAAAAGGGC

If any of the above Cas9 sequences are fused with a peptide or polypeptide at the C-terminus, it is understood that the stop codon will be removed.

Other Cas Molecules and Cas Polypeptides

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLoS COMPUTATIONAL BIOLOGY 2005, 1(6): e60 and Makarova et al., NATURE REVIEW MICROBIOLOGY 2011, 9:467-477, the contents of both references are incorporated herein by reference in their entirety. Exemplary Cas molecules (and Cas systems) are also shown in Table 13.

TABLE 13
Cas Systems

| | | | Structure of | Families (and | |
|---|---|---|---|---|---|
| | | | encoded | superfamily) of | |
| Gene | System type | Name from | protein (PDB | encoded | |
| name^{‡} | or subtype | Haft et al.^{§} | accessions)^{¶} | protein^{#}** | Representatives |
| cas1 | Type I | cas1 | 3GOD, 3LFX | COG1518 | SERP2463, SPy1047 |
| | Type II | | and 2YZS | | and ygbT |
| | Type III | | | | |
| cas2 | Type I | cas2 | 2IVY, 2I8E and | COG1343 and | SERP2462, SPy1048, |
| | Type II | | 3EXC | COG3512 | SPy1723 (N-terminal |
| | Type III | | | | domain) and ygbF |
| cas3′ | Type I^{‡‡} | cas3 | NA | COG1203 | APE1232 and ygcB |
| cas3″ | Subtype I-A | NA | NA | COG2254 | APE1231 and |
| | Subtype I-B | | | | BH0336 |
| cas4 | Subtype I-A | cas4 and csa1 | NA | COG1468 | APE1239 and |
| | Subtype I-B | | | | BH0340 |
| | Subtype I-C | | | | |
| | Subtype I-D | | | | |
| | Subtype II-B | | | | |
| cas5 | Subtype I-A | cas5a, cas5d, | 3KG4 | COG1688 | APE1234, BH0337, |
| | Subtype I-B | cas5e, cas5h, | | (RAMP) | devS and ygcI |
| | Subtype I-C | cas5p, cas5t | | | |
| | Subtype I-E | and cmx5 | | | |
| cas6 | Subtype I-A | cas6 and cmx6 | 3I4H | COG1583 and | PF1131 and slr7014 |
| | Subtype I-B | | | COG5551 | |
| | Subtype I-D | | | (RAMP) | |
| | Subtype III- | | | | |
| | A Subtype | | | | |
| | III-B | | | | |
| cas6e | Subtype I-E | cse3 | 1WJ9 | (RAMP) | ygcH |
| cas6f | Subtype I-F | csy4 | 2XLJ | (RAMP) | y1727 |
| cas7 | Subtype I-A | csa2, csd2, | NA | COG1857 and | devR and ygcJ |
| | Subtype I-B | cse4, csh2, | | COG3649 | |
| | Subtype I-C | csp1 and cst2 | | (RAMP) | |
| | Subtype I-E | | | | |
| cas8a1 | Subtype I- | cmx1, cst1, | NA | BH0338-like | LA3191^{§§} and |
| | A^{‡‡} | csx8, csx13 | | | PG2018^{§§} |
| | | and CXXC- | | | |
| | | CXXC | | | |
| cas8a2 | Subtype I- | csa4 and csx9 | NA | PH0918 | AF0070, AF1873, |
| | A^{‡‡} | | | | MJ0385, PF0637, |
| | | | | | PH0918 and |
| | | | | | SSO1401 |
| cas8b | Subtype I- | csh1 and | NA | BH0338-like | MTH1090 and |
| | B^{‡‡} | TM1802 | | | TM1802 |
| cas8c | Subtype I- | csd1 and csp2 | NA | BH0338-like | BH0338 |
| | C^{‡‡} | | | | |
| cas9 | Type II^{‡‡} | csn1 and csx12 | NA | COG3513 | FTN_0757 and |
| | | | | | SPy1046 |
| cas10 | Type III^{‡‡} | cmr2, csm1 | NA | COG1353 | MTH326, Rv2823c^{§§} |
| | | and csx11 | | | and TM1794^{§§} |
| cas10d | Subtype I- | csc3 | NA | COG1353 | slr7011 |
| | D^{‡‡} | | | | |
| csy1 | Subtype I- | csy1 | NA | y1724-like | y1724 |
| | F^{‡‡} | | | | |
| csy2 | Subtype I-F | csy2 | NA | (RAMP) | y1725 |
| csy3 | Subtype I-F | csy3 | NA | (RAMP) | y1726 |
| cse1 | Subtype I- | cse1 | NA | YgcL-like | ygcL |
| | E^{‡‡} | | | | |
| cse2 | Subtype I-E | cse2 | 2ZCA | YgcK-like | ygcK |
| csc1 | Subtype I-D | csc1 | NA | alr1563-like | alr1563 |
| | | | | (RAMP) | |
| csc2 | Subtype I-D | csc1 and csc2 | NA | COG1337 | slr7012 |
| | | | | (RAMP) | |
| csa5 | Subtype I-A | csa5 | NA | AF1870 | AF1870, MJ0380, |
| | | | | | PF0643 and |
| | | | | | SSO1398 |
| csn2 | Subtype II-A | csn2 | NA | SPy1049-like | SPy1049 |
| csm2 | Subtype III- | csm2 | NA | COG1421 | MTH1081 and |
| | A^{‡‡} | | | | SERP2460 |
| csm3 | Subtype III-A | csc2 and csm3 | NA | COG1337 | MTH1080 and |
| | | | | (RAMP) | SERP2459 |
| csm4 | Subtype III-A | csm4 | NA | COG1567 | MTH1079 and |
| | | | | (RAMP) | SERP2458 |
| csm5 | Subtype III-A | csm5 | NA | COG1332 | MTH1078 and |
| | | | | (RAMP) | SERP2457 |
| csm6 | Subtype III-A | APE2256 and | 2WTE | COG1517 | APE2256 and |
| | | csm6 | | | SSO1445 |
| cmr1 | Subtype III-B | cmr1 | NA | COG1367 | PF1130 |
| | | | | (RAMP) | |
| cmr3 | Subtype III-B | cmr3 | NA | COG1769 | PF1128 |
| | | | | (RAMP) | |
| cmr4 | Subtype III-B | cmr4 | NA | COG1336 | PF1126 |
| | | | | (RAMP) | |
| cmr5 | Subtype III- | cmr5 | 2ZOP and | COG3337 | MTH324 and |
| | B^{‡‡} | | 2OEB | | PF1125 |
| cmr6 | Subtype III-B | cmr6 | NA | COG1604 | PF1124 |
| | | | | (RAMP) | |
| csb1 | Subtype I-U | GSU0053 | NA | (RAMP) | Balac_1306 and |
| | | | | | GSU0053 |
| csb2 | Subtype I- | NA | NA | (RAMP) | Balac_1305 and |
| | U^{§§} | | | | GSU0054 |
| csb3 | Subtype I-U | NA | NA | (RAMP) | Balac_1303^{§§} |
| csx17 | Subtype I-U | NA | NA | NA | Btus_2683 |
| csx14 | Subtype I-U | NA | NA | NA | GSU0052 |
| csx10 | Subtype I-U | csx10 | NA | (RAMP) | Caur_2274 |
| csx16 | Subtype III-U | VVA1548 | NA | NA | VVA1548 |
| csaX | Subtype III-U | csaX | NA | NA | SSO1438 |
| csx3 | Subtype III-U | csx3 | NA | NA | AF1864 |
| csx1 | Subtype III-U | csa3, csx1, | 1XMX and | COG1517 and | MJ1666, NE0113, |
| | | csx2, DXTHG, | 2I71 | COG4006 | PF1127 and TM1812 |
| | | NE0113 and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

### IV. Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule are described, e.g., in Jinek et al., SCIENCE 2012, 337(6096):816-821.

Binding and Cleavage Assay: Testing the Endonuclease Activity of Cas9 Molecule

The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and ˜3-6 pmol (˜20-40 mCi) [γ-32P]-ATP in 1×T4 polynucleotide kinase reaction buffer at 37° C. for 30 min, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl2, 1 mM DTT, 5% glycerol) in a total volume of 9 μl. Reactions are initiated by the addition of 1 μl target DNA (10 nM) and incubated for 1 h at 37° C. Reactions are quenched by the addition of 20 μl of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphor imaging. The resulting cleavage products indicate that whether the complementary strand, the non-complementary strand, or both, are cleaved.

One or both of these assays can be used to evaluate the suitability of a candidate gRNA molecule or candidate Cas9 molecule.

Binding Assay: Testing the Binding of Cas9 Molecule to Target DNA

Exemplary methods for evaluating the binding of Cas9 molecule to target DNA are described, e.g., in Jinek et al., SCIENCE 2012; 337(6096):816-821.

For example, in an electrophoretic mobility shift assay, target DNA duplexes are formed by mixing of each strand (10 nmol) in deionized water, heating to 95° C. for 3 min and slow cooling to room temperature. All DNAs are purified on 8% native gels containing 1×TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-32P]-ATP using T4 polynucleotide kinase for 30 min at 37° C. Polynucleotide kinase is heat denatured at 65° C. for 20 min, and unincorporated radiolabel is removed using a column. Binding assays are performed in buffer containing 20 mM HEPES pH 7.5, 100 mM KCl, 5 mM MgCl₂, 1 mM DTT and 10% glycerol in a total volume of 10 μl. Cas9 protein molecule is programmed with equimolar amounts of pre-annealed gRNA molecule and titrated from 100 pM to 1 μM. Radiolabeled DNA is added to a final concentration of 20 pM. Samples are incubated for 1 h at 37° C. and resolved at 4° C. on an 8% native polyacrylamide gel containing 1×TBE and 5 mM MgCl₂. Gels are dried and DNA visualized by phosphor imaging.

Differential Scanning Flourimetry (DSF)

The thermostability of Cas9-gRNA ribonucleoprotein (RNP) complexes can be measured via DSF. This technique measures the thermostability of a protein, which can increase under favorable conditions such as the addition of a binding RNA molecule, e.g., a gRNA.

The assay is performed using two different protocols, one to test the best stoichiometric ratio of gRNA:Cas9 protein and another to determine the best solution conditions for RNP formation.

To determine the best solution to form RNP complexes, a 2 uM solution of Cas9 in water+10× SYPRO Orange® (Life Technologies cat#S-6650) and dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

The second assay consists of mixing various concentrations of gRNA with 2 uM Cas9 in optimal buffer from assay 1 above and incubating at RT for 10′ in a 384 well plate. An equal volume of optimal buffer+10× SYPRO Orange® (Life Technologies cat#S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

### V. Genome Editing Approaches

Described herein are methods for targeted knockout of the CCR5 gene, e.g., one or both alleles of the CCR5 gene, e.g., using one or more of the approaches or pathways described herein, e.g., using NHEJ. Described herein are also methods for targeted knockdown of the CCR5 gene.

V.1 NHEJ Approaches for Gene Targeting

As described herein, nuclease-induced non-homologous end-joining (NHEJ) can be used to target gene-specific knockouts. Nuclease-induced NHEJ can also be used to remove (e.g., delete) sequence insertions in a gene of interest.

While not wishing to be bound by theory, it is believed that, in an embodiment, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein.

The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to the early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

Placement of Double Strand or Single Strand Breaks Relative to the Target Position

In an embodiment, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In an embodiment, the cleavage site is between 0-30 bp away from the target position (e.g., less than 30, 25, 20, 15, 10, 9, 8, 7, 6, 5, 4, 3, 2 or 1 bp from the target position).

In an embodiment, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In an embodiment, the gRNAs are configured to position cuts at the same position, or within a few nucleotides of one another, on different strands, essentially mimicking a double strand break. In an embodiment, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In an embodiment, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In one embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

V.2 Single-Strand Annealing

Single strand annealing (SSA) is another DNA repair process that repairs a double-strand break between two repeat sequences present in a target nucleic acid. Repeat sequences utilized by the SSA pathway are generally greater than 30 nucleotides in length. Resection at the break ends occurs to reveal repeat sequences on both strands of the target nucleic acid. After resection, single strand overhangs containing the repeat sequences are coated with RPA protein to prevent the repeats sequences from inappropriate annealing, e.g., to themselves. RAD52 binds to and each of the repeat sequences on the overhangs and aligns the sequences to enable the annealing of the complementary repeat sequences. After annealing, the single-strand flaps of the overhangs are cleaved. New DNA synthesis fills in any gaps, and ligation restores the DNA duplex. As a result of the processing, the DNA sequence between the two repeats is deleted. The length of the deletion can depend on many factors including the location of the two repeats utilized, and the pathway or processivity of the resection.

In contrast to HDR pathways, SSA does not require a template nucleic acid to alter or correct a target nucleic acid sequence. Instead, the complementary repeat sequence is utilized.

V.3 Other DNA Repair Pathways

SSBR (Single Strand Break Repair)

Single-stranded breaks (SSB) in the genome are repaired by the SSBR pathway, which is a distinct mechanism from the DSB repair mechanisms discussed above. The SSBR pathway has four major stages: SSB detection, DNA end processing, DNA gap filling, and DNA ligation. A more detailed explanation is given in Caldecott, Nature Reviews Genetics 9, 619-631 (August 2008), and a summary is given here.

In the first stage, when a SSB forms, PARP1 and/or PARP2 recognize the break and recruit repair machinery. The binding and activity of PARP1 at DNA breaks is transient and it seems to accelerate SSBr by promoting the focal accumulation or stability of SSBr protein complexes at the lesion. Arguably the most important of these SSBr proteins is XRCC1, which functions as a molecular scaffold that interacts with, stabilizes, and stimulates multiple enzymatic components of the SSBr process including the protein responsible for cleaning the DNA 3′ and 5′ ends. For instance, XRCC1 interacts with several proteins (DNA polymerase beta, PNK, and three nucleases, APE1, APTX, and APLF) that promote end processing. APE1 has endonuclease activity. APLF exhibits endonuclease and 3′ to 5′ exonuclease activities. APTX has endonuclease and 3′ to 5′ exonuclease activity.

This end processing is an important stage of SSBR since the 3′- and/or 5′-termini of most, if not all, SSBs are ‘damaged’. End processing generally involves restoring a damaged 3′-end to a hydroxylated state and and/or a damaged 5′ end to a phosphate moiety, so that the ends become ligation-competent. Enzymes that can process damaged 3′ termini include PNKP, APE1, and TDP1. Enzymes that can process damaged 5′ termini include PNKP, DNA polymerase beta, and APTX. LIG3 (DNA ligase III) can also participate in end processing. Once the ends are cleaned, gap filling can occur.

At the DNA gap filling stage, the proteins typically present are PARP1, DNA polymerase beta, XRCC1, FEN1 (flap endonuclease 1), DNA polymerase delta/epsilon, PCNA, and LIG1. There are two ways of gap filling, the short patch repair and the long patch repair. Short patch repair involves the insertion of a single nucleotide that is missing. At some SSBs, “gap filling” might continue displacing two or more nucleotides (displacement of up to 12 bases have been reported). FEN1 is an endonuclease that removes the displaced 5′-residues. Multiple DNA polymerases, including Pol β, are involved in the repair of SSBs, with the choice of DNA polymerase influenced by the source and type of SSB.

In the fourth stage, a DNA ligase such as LIG1 (Ligase I) or LIG3 (Ligase III) catalyzes joining of the ends. Short patch repair uses Ligase III and long patch repair uses Ligase I.

Sometimes, SSBR is replication-coupled. This pathway can involve one or more of CtIP, MRN, ERCC1, and FEN1. Additional factors that may promote SSBR include: aPARP, PARP1, PARP2, PARG, XRCC1, DNA polymerase b, DNA polymerase d, DNA polymerase e, PCNA, LIG1, PNK, PNKP, APE1, APTX, APLF, TDP1, LIG3, FEN1, CtIP, MRN, and ERCC1.

MMR (Mismatch Repair)

Cells contain three excision repair pathways: MMR, BER, and NER. The excision repair pathways have a common feature in that they typically recognize a lesion on one strand of the DNA, then exo/endonucleases remove the lesion and leave a 1-30 nucleotide gap that is sub-sequentially filled in by DNA polymerase and finally sealed with ligase. A more complete picture is given in Li, Cell Research (2008) 18:85-98, and a summary is provided here.

Mismatch repair (MMR) operates on mispaired DNA bases.

The MSH2/6 or MSH2/3 complexes both have ATPases activity that plays an important role in mismatch recognition and the initiation of repair. MSH2/6 preferentially recognizes base-base mismatches and identifies mispairs of 1 or 2 nucleotides, while MSH2/3 preferentially recognizes larger ID mispairs.

hMLH1 heterodimerizes with hPMS2 to form hMutL α which possesses an ATPase activity and is important for multiple steps of MMR. It possesses a PCNA/replication factor C (RFC)-dependent endonuclease activity which plays an important role in 3′ nick-directed MMR involving EXO1. (EXO1 is a participant in both HR and MMR.) It regulates termination of mismatch-provoked excision. Ligase I is the relevant ligase for this pathway. Additional factors that may promote MMR include: EXO1, MSH2, MSH3, MSH6, MLH1, PMS2, MLH3, DNA Pol d, RPA, HMGB1, RFC, and DNA ligase I.

Base Excision Repair (BER)

The base excision repair (BER) pathway is active throughout the cell cycle; it is responsible primarily for removing small, non-helix-distorting base lesions from the genome. In contrast, the related Nucleotide Excision Repair pathway (discussed in the next section) repairs bulky helix-distorting lesions. A more detailed explanation is given in Caldecott, Nature Reviews Genetics 9, 619-631 (August 2008), and a summary is given here.

Upon DNA base damage, base excision repair (BER) is initiated and the process can be simplified into five major steps: (a) removal of the damaged DNA base; (b) incision of the subsequent a basic site; (c) clean-up of the DNA ends; (d) insertion of the correct nucleotide into the repair gap; and (e) ligation of the remaining nick in the DNA backbone. These last steps are similar to the SSBR.

In the first step, a damage-specific DNA glycosylase excises the damaged base through cleavage of the N-glycosidic bond linking the base to the sugar phosphate backbone. Then AP endonuclease-1 (APE1) or bifunctional DNA glycosylases with an associated lyase activity incised the phosphodiester backbone to create a DNA single strand break (SSB). The third step of BER involves cleaning-up of the DNA ends. The fourth step in BER is conducted by Pol that adds a new complementary nucleotide into the repair gap and in the final step XRCC1/Ligase III seals the remaining nick in the DNA backbone. This completes the short-patch BER pathway in which the majority (˜80%) of damaged DNA bases are repaired. However, if the 5′-ends in step 3 are resistant to end processing activity, following one nucleotide insertion by Pol β there is then a polymerase switch to the replicative DNA polymerases, Pol δ/ε, which then add ˜2-8 more nucleotides into the DNA repair gap. This creates a 5′-flap structure, which is recognized and excised by flap endonuclease-1 (FEN-1) in association with the processivity factor proliferating cell nuclear antigen (PCNA). DNA ligase I then seals the remaining nick in the DNA backbone and completes long-patch BER. Additional factors that may promote the BER pathway include: DNA glycosylase, APE1, Polb, Pold, Pole, XRCC1, Ligase III, FEN-1, PCNA, RECQL4, WRN, MYH, PNKP, and APTX.

Nucleotide Excision Repair (NER)

Nucleotide excision repair (NER) is an important excision mechanism that removes bulky helix-distorting lesions from DNA. Additional details about NER are given in Marteijn et al., Nature Reviews Molecular Cell Biology 15, 465-481 (2014), and a summary is given here. NER a broad pathway encompassing two smaller pathways: global genomic NER (GG-NER) and transcription coupled repair NER (TC-NER). GG-NER and TC-NER use different factors for recognizing DNA damage. However, they utilize the same machinery for lesion incision, repair, and ligation.

Once damage is recognized, the cell removes a short single-stranded DNA segment that contains the lesion. Endonucleases XPF/ERCC1 and XPG (encoded by ERCC5) remove the lesion by cutting the damaged strand on either side of the lesion, resulting in a single-strand gap of 22-30 nucleotides. Next, the cell performs DNA gap filling synthesis and ligation. Involved in this process are: PCNA, RFC, DNA Pol δ, DNA Pol ε or DNA Pol κ, and DNA ligase I or XRCC1/Ligase III. Replicating cells tend to use DNA pol ε and DNA ligase I, while non-replicating cells tend to use DNA Pol δ, DNA Pol κ, and the XRCC1/Ligase III complex to perform the ligation step.

NER can involve the following factors: XPA-G, POLH, XPF, ERCC1, XPA-G, and LIG1. Transcription-coupled NER (TC-NER) can involve the following factors: CSA, CSB, XPB, XPD, XPG, ERCC1, and TTDA. Additional factors that may promote the NER repair pathway include XPA-G, POLH, XPF, ERCC1, XPA-G, LIG1, CSA, CSB, XPA, XPB, XPC, XPD, XPF, XPG, TTDA, UVSSA, USP7, CETN2, RAD23B, UV-DDB, CAK subcomplex, RPA, and PCNA.

Interstrand Crosslink (ICL)

A dedicated pathway called the ICL repair pathway repairs interstrand crosslinks. Interstrand crosslinks, or covalent crosslinks between bases in different DNA strand, can occur during replication or transcription. ICL repair involves the coordination of multiple repair processes, in particular, nucleolytic activity, translesion synthesis (TLS), and HDR. Nucleases are recruited to excise the ICL on either side of the crosslinked bases, while TLS and HDR are coordinated to repair the cut strands. ICL repair can involve the following factors: endonucleases, e.g., XPF and RAD51C, endonucleases such as RAD51, translesion polymerases, e.g., DNA polymerase zeta and Rev1), and the Fanconi anemia (FA) proteins, e.g., FancJ.

Other Pathways

Several other DNA repair pathways exist in mammals.

Translesion synthesis (TLS) is a pathway for repairing a single stranded break left after a defective replication event and involves translesion polymerases, e.g., DNA polζ and Rev1.

Error-free postreplication repair (PRR) is another pathway for repairing a single stranded break left after a defective replication event.

V.4 Targeted Knockdown

Unlike CRISPR/Cas-mediated gene knockout, which permanently eliminates expression by mutating the gene at the DNA level, CRISPR/Cas knockdown allows for temporary reduction of gene expression through the use of artificial transcription factors. Mutating key residues in both DNA cleavage domains of the Cas9 protein (e.g. the D10A and H840A mutations) results in the generation of a catalytically inactive Cas9 (eiCas9 which is also known as dead Cas9 or dCas9) molecule. A catalytically inactive Cas9 complexes with a gRNA and localizes to the DNA sequence specified by that gRNA's targeting domain, however, it does not cleave the target DNA. Fusion of the dCas9 to an effector domain, e.g., a transcription repression domain, enables recruitment of the effector to any DNA site specified by the gRNA. Although an enzymatically inactive (eiCas9) Cas9 molecule itself can block transcription when recruited to early regions in the coding sequence, more robust repression can be achieved by fusing a transcriptional repression domain (for example KRAB, SID or ERD) to the Cas9 and recruiting it to the target knockdown position, e.g., within 1000 bp of sequence 3′ of the start codon or within 500 bp of a promoter region 5′ of the start codon of a gene. It is likely that targeting DNAseI hypersensitive sites (DHSs) of the promoter may yield more efficient gene repression or activation because these regions are more likely to be accessible to the Cas9 protein and are also more likely to harbor sites for endogenous transcription factors. Especially for gene repression, it is contemplated herein that blocking the binding site of an endogenous transcription factor would aid in downregulating gene expression. In an embodiment, one or more eiCas9 molecules may be used to block binding of one or more endogenous transcription factors. In another embodiment, an eiCas9 molecule can be fused to a chromatin modifying protein. Altering chromatin status can result in decreased expression of the target gene. One or more eiCas9 molecules fused to one or more chromatin modifying proteins may be used to alter chromatin status.

In an embodiment, a gRNA molecule can be targeted to a known transcription response elements (e.g., promoters, enhancers, etc.), a known upstream activating sequences (UAS), and/or sequences of unknown or known function that are suspected of being able to control expression of the target DNA.

CRISPR/Cas-mediated gene knockdown can be used to reduce expression of an unwanted allele or transcript. Contemplated herein are scenarios wherein permanent destruction of the gene is not ideal. In these scenarios, site-specific repression may be used to temporarily reduce or eliminate expression. It is also contemplated herein that the off-target effects of a Cas-repressor may be less severe than those of a Cas-nuclease as a nuclease can cleave any DNA sequence and cause mutations whereas a Cas-repressor may only have an effect if it targets the promoter region of an actively transcribed gene. However, while nuclease-mediated knockout is permanent, repression may only persist as long as the Cas-repressor is present in the cells. Once the repressor is no longer present, it is likely that endogenous transcription factors and gene regulatory elements would restore expression to its natural state.

V.5 Examples of gRNAs in Genome Editing Methods

gRNA molecules as described herein can be used with Cas9 molecules that generate a double strand break or a single strand break to alter the sequence of a target nucleic acid, e.g., a target position or target genetic signature. gRNA molecules useful in these methods are described below.

In an embodiment, the gRNA, e.g., a chimeric gRNA, is configured such that it comprises one or more of the following properties;

a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;

b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

c)
(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or
(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In an embodiment, the gRNA is configured such that it comprises properties: a and b(i).

In an embodiment, the gRNA is configured such that it comprises properties: a and b(ii).

In an embodiment, the gRNA is configured such that it comprises properties: a and b(iii).

In an embodiment, the gRNA is configured such that it comprises properties: a and b(iv).

In an embodiment, the gRNA is configured such that it comprises properties: a and b(v).

In an embodiment, the gRNA is configured such that it comprises properties: a and b(vi).

In an embodiment, the gRNA is configured such that it comprises properties: a and b(vii).

In an embodiment, the gRNA is configured such that it comprises properties: a and b(viii).

In an embodiment, the gRNA is configured such that it comprises properties: a and b(ix).

In an embodiment, the gRNA is configured such that it comprises properties: a and b(x).

In an embodiment, the gRNA is configured such that it comprises properties: a and b(xi).

In an embodiment, the gRNA is configured such that it comprises properties: a and c.

In an embodiment, the gRNA is configured such that in comprises properties: a, b, and c.

In an embodiment, the gRNA is configured such that in comprises properties: a(i), b(i), and c(i).

In an embodiment, the gRNA is configured such that in comprises properties: a(i), b(i), and c(ii).

In an embodiment, the gRNA is configured such that in comprises properties: a(i), b(ii), and c(i).

In an embodiment, the gRNA is configured such that in comprises properties: a(i), b(ii), and c(ii).

In an embodiment, the gRNA is configured such that in comprises properties: a(i), b(iii), and c(i).

In an embodiment, the gRNA is configured such that in comprises properties: a(i), b(iii), and c(ii).

In an embodiment, the gRNA is configured such that in comprises properties: a(i), b(iv), and c(i).

In an embodiment, the gRNA is configured such that in comprises properties: a(i), b(iv), and c(ii).

In an embodiment, the gRNA is configured such that in comprises properties: a(i), b(v), and c(i).

In an embodiment, the gRNA is configured such that in comprises properties: a(i), b(v), and c(ii).

In an embodiment, the gRNA is configured such that in comprises properties: a(i), b(vi), and c(i).

In an embodiment, the gRNA is configured such that in comprises properties: a(i), b(vi), and c(ii).

In an embodiment, the gRNA is configured such that in comprises properties: a(i), b(vii), and c(i).

In an embodiment, the gRNA is configured such that in comprises properties: a(i), b(vii), and c(ii).

In an embodiment, the gRNA is configured such that in comprises properties: a(i), b(viii), and c(i).

In an embodiment, the gRNA is configured such that in comprises properties: a(i), b(viii), and c(ii).

In an embodiment, the gRNA is configured such that in comprises properties: a(i), b(ix), and c(i).

In an embodiment, the gRNA is configured such that in comprises properties: a(i), b(ix), and c(ii).

In an embodiment, the gRNA is configured such that in comprises properties: a(i), b(x), and c(i).

In an embodiment, the gRNA is configured such that in comprises properties: a(i), b(x), and c(ii).

In an embodiment, the gRNA is configured such that in comprises properties: a(i), b(xi), and c(i).

In an embodiment, the gRNA is configured such that in comprises properties: a(i), b(xi), and c(ii).

In an embodiment, the gRNA, e.g., a chimeric gRNA, is configured such that it comprises one or more of the following properties;

a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

c)
(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or
(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In an embodiment, the gRNA is configured such that it comprises properties: a and b(i).

In an embodiment, the gRNA is configured such that it comprises properties: a and b(ii).

In an embodiment, the gRNA is configured such that it comprises properties: a and b(iii).

In an embodiment, the gRNA is configured such that it comprises properties: a and b(iv).

In an embodiment, the gRNA is configured such that it comprises properties: a and b(v).

In an embodiment, the gRNA is configured such that it comprises properties: a and b(vi).

In an embodiment, the gRNA is configured such that it comprises properties: a and b(vii).

In an embodiment, the gRNA is configured such that it comprises properties: a and b(viii).

In an embodiment, the gRNA is configured such that it comprises properties: a and b(ix).

In an embodiment, the gRNA is configured such that it comprises properties: a and b(x).

In an embodiment, the gRNA is configured such that it comprises properties: a and b(xi).

In an embodiment, the gRNA is configured such that it comprises properties: a and c.

In an embodiment, the gRNA is configured such that in comprises properties: a, b, and c.

In an embodiment, the gRNA is configured such that in comprises properties: a(i), b(i), and c(i).

In an embodiment, the gRNA is configured such that in comprises properties: a(i), b(i), and c(ii).

In an embodiment, the gRNA is configured such that in comprises properties: a(i), b(ii), and c(i).

In an embodiment, the gRNA is configured such that in comprises properties: a(i), b(ii), and c(ii).

In an embodiment, the gRNA is configured such that in comprises properties: a(i), b(iii), and c(i).

In an embodiment, the gRNA is configured such that in comprises properties: a(i), b(iii), and c(ii).

In an embodiment, the gRNA is configured such that in comprises properties: a(i), b(iv), and c(i).

In an embodiment, the gRNA is configured such that in comprises properties: a(i), b(iv), and c(ii).

In an embodiment, the gRNA is configured such that in comprises properties: a(i), b(v), and c(i).

In an embodiment, the gRNA is configured such that in comprises properties: a(i), b(v), and c(ii).

In an embodiment, the gRNA is configured such that in comprises properties: a(i), b(vi), and c(i).

In an embodiment, the gRNA is configured such that in comprises properties: a(i), b(vi), and c(ii).

In an embodiment, the gRNA is configured such that in comprises properties: a(i), b(vii), and c(i).

In an embodiment, the gRNA is configured such that in comprises properties: a(i), b(vii), and c(ii).

In an embodiment, the gRNA is configured such that in comprises properties: a(i), b(viii), and c(i).

In an embodiment, the gRNA is configured such that in comprises properties: a(i), b(viii), and c(ii).

In an embodiment, the gRNA is configured such that in comprises properties: a(i), b(ix), and c(i).

In an embodiment, the gRNA is configured such that in comprises properties: a(i), b(ix), and c(ii).

In an embodiment, the gRNA is configured such that in comprises properties: a(i), b(x), and c(i).

In an embodiment, the gRNA is configured such that in comprises properties: a(i), b(x), and c(ii).

In an embodiment, the gRNA is configured such that in comprises properties: a(i), b(xi), and c(i).

In an embodiment, the gRNA is configured such that in comprises properties: a(i), b(xi), and c(ii).

In an embodiment, the gRNA is used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNA is used with a Cas9 nickase molecule having RuvC activity, e.g., a Cas9 molecule having the HNH activity inactivated, e.g., a Cas9 molecule having a mutation at H840, e.g., the H840A.

In an embodiment, the gRNAs are used with a Cas9 nickase molecule having RuvC activity, e.g., a Cas9 molecule having the HNH activity inactivated, e.g., a Cas9 molecule having a mutation at H863, e.g., the N863A.

In an embodiment, a pair of gRNAs, e.g., a pair of chimeric gRNAs, comprising a first and a second gRNA, is configured such that they comprises one or more of the following properties;

a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides;

c) for one or both:
(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, or a sequence that differs by no more than 1, 2, 3, 4, 5; 6, 7, 8, 9 or 10 nucleotides therefrom; or
(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain;

d) the gRNAs are configured such that, when hybridized to target nucleic acid, they are separated by 0-50, 0-100, 0-200, at least 10, at least 20, at least 30 or at least 50 nucleotides;

e) the breaks made by the first gRNA and second gRNA are on different strands; and

f) the PAMs are facing outwards.

In an embodiment, one or both of the gRNAs is configured such that it comprises properties: a and b(i).

In an embodiment, one or both of the gRNAs is configured such that it comprises properties: a and b(ii).

In an embodiment, one or both of the gRNAs is configured such that it comprises properties: a and b(iii).

In an embodiment, one or both of the gRNAs is configured such that it comprises properties: a and b(iv).

In an embodiment, one or both of the gRNAs is configured such that it comprises properties: a and b(v).

In an embodiment, one or both of the gRNAs is configured such that it comprises properties: a and b(vi).

In an embodiment, one or both of the gRNAs is configured such that it comprises properties: a and b(vii).

In an embodiment, one or both of the gRNAs is configured such that it comprises properties: a and b(viii).

In an embodiment, one or both of the gRNAs is configured such that it comprises properties: a and b(ix).

In an embodiment, one or both of the gRNAs is configured such that it comprises properties: a and b(x).

In an embodiment, one or both of the gRNAs is configured such that it comprises properties: a and b(xi).

In an embodiment, one or both of the gRNAs configured such that it comprises properties: a and c.

In an embodiment, one or both of the gRNAs is configured such that it comprises properties: a, b, and c.

In an embodiment, one or both of the gRNAs is configured such that it comprises properties: a(i), b(i), and c(i).

In an embodiment, one or both of the gRNAs is configured such that it comprises properties: a(i), b(i), and c(ii).

In an embodiment, one or both of the gRNAs is configured such that it comprises properties: a(i), b(i), c, and d.

In an embodiment, one or both of the gRNAs is configured such that it comprises properties: a(i), b(i), c, and e.

In an embodiment, one or both of the gRNAs is configured such that it comprises properties: a(i), b(i), c, d, and e.

In an embodiment, one or both of the gRNAs is configured such that it comprises properties: a(i), b(ii), and c(i).

In an embodiment, one or both of the gRNAs is configured such that it comprises properties: a(i), b(ii), and c(ii).

In an embodiment, one or both of the gRNAs is configured such that it comprises properties: a(i), b(ii), c, and d.

In an embodiment, one or both of the gRNAs is configured such that it comprises properties: a(i), b(ii), c, and e.

In an embodiment, one or both of the gRNAs is configured such that it comprises properties: a(i), b(ii), c, d, and e.

In an embodiment, one or both of the gRNAs is configured such that it comprises properties: a(i), b(iii), and c(i).

In an embodiment, one or both of the gRNAs is configured such that it comprises properties: a(i), b(iii), and c(ii).

In an embodiment, one or both of the gRNAs is configured such that it comprises properties: a(i), b(iii), c, and d.

In an embodiment, one or both of the gRNAs is configured such that it comprises properties: a(i), b(iii), c, and e.

In an embodiment, one or both of the gRNAs is configured such that it comprises properties: a(i), b(iii), c, d, and e.

In an embodiment, one or both of the gRNAs is configured such that it comprises properties: a(i), b(iv), and c(i).

In an embodiment, one or both of the gRNAs is configured such that it comprises properties: a(i), b(iv), and c(ii).

In an embodiment, one or both of the gRNAs is configured such that it comprises properties: a(i), b(iv), c, and d.

In an embodiment, one or both of the gRNAs is configured such that it comprises properties: a(i), b(iv), c, and e.

In an embodiment, one or both of the gRNAs is configured such that it comprises properties: a(i), b(iv), c, d, and e.

In an embodiment, one or both of the gRNAs is configured such that it comprises properties: a(i), b(v), and c(i).

In an embodiment, one or both of the gRNAs is configured such that it comprises properties: a(i), b(v), and c(ii).

In an embodiment, one or both of the gRNAs is configured such that it comprises properties: a(i), b(v), c, and d.

In an embodiment, one or both of the gRNAs is configured such that it comprises properties: a(i), b(v), c, and e.

In an embodiment, one or both of the gRNAs is configured such that it comprises properties: a(i), b(v), c, d, and e.

In an embodiment, one or both of the gRNAs is configured such that it comprises properties: a(i), b(vi), and c(i).

In an embodiment, one or both of the gRNAs is configured such that it comprises properties: a(i), b(vi), and c(ii).

In an embodiment, one or both of the gRNAs is configured such that it comprises properties: a(i), b(vi), c, and d.

In an embodiment, one or both of the gRNAs is configured such that it comprises properties: a(i), b(vi), c, and e.

In an embodiment, one or both of the gRNAs is configured such that it comprises properties: a(i), b(vi), c, d, and e.

In an embodiment, one or both of the gRNAs is configured such that it comprises properties: a(i), b(vii), and c(i).

In an embodiment, one or both of the gRNAs is configured such that it comprises properties: a(i), b(vii), and c(ii).

In an embodiment, one or both of the gRNAs is configured such that it comprises properties: a(i), b(vii), c, and d.

In an embodiment, one or both of the gRNAs is configured such that it comprises properties: a(i), b(vii), c, and e.

In an embodiment, one or both of the gRNAs is configured such that it comprises properties: a(i), b(vii), c, d, and e.

In an embodiment, one or both of the gRNAs is configured such that it comprises properties: a(i), b(viii), and c(i).

In an embodiment, one or both of the gRNAs is configured such that it comprises properties: a(i), b(viii), and c(ii).

In an embodiment, one or both of the gRNAs is configured such that it comprises properties: a(i), b(viii), c, and d.

In an embodiment, one or both of the gRNAs is configured such that it comprises properties: a(i), b(viii), c, and e.

In an embodiment, one or both of the gRNAs is configured such that it comprises properties: a(i), b(viii), c, d, and e.

In an embodiment, one or both of the gRNAs is configured such that it comprises properties: a(i), b(ix), and c(i).

In an embodiment, one or both of the gRNAs is configured such that it comprises properties: a(i), b(ix), and c(ii).

In an embodiment, one or both of the gRNAs is configured such that it comprises properties: a(i), b(ix), c, and d.

In an embodiment, one or both of the gRNAs is configured such that it comprises properties: a(i), b(ix), c, and e.

In an embodiment, one or both of the gRNAs is configured such that it comprises properties: a(i), b(ix), c, d, and e.

In an embodiment, one or both of the gRNAs is configured such that it comprises properties: a(i), b(x), and c(i).

In an embodiment, one or both of the gRNAs is configured such that it comprises properties: a(i), b(x), and c(ii).

In an embodiment, one or both of the gRNAs is configured such that it comprises properties: a(i), b(x), c, and d.

In an embodiment, one or both of the gRNAs is configured such that it comprises properties: a(i), b(x), c, and e.

In an embodiment, one or both of the gRNAs is configured such that it comprises properties: a(i), b(x), c, d, and e.

In an embodiment, one or both of the gRNAs is configured such that it comprises properties: a(i), b(xi), and c(i).

In an embodiment, one or both of the gRNAs is configured such that it comprises properties: a(i), b(xi), and c(ii).

In an embodiment, one or both of the gRNAs is configured such that it comprises properties: a(i), b(xi), c, and d.

In an embodiment, one or both of the gRNAs is configured such that it comprises properties: a(i), b(xi), c, and e.

In an embodiment, one or both of the gRNAs is configured such that it comprises properties: a(i), b(xi), c, d, and e.

In an embodiment, the gRNAs are used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNAs are used with a Cas9 nickase molecule having RuvC activity, e.g., a Cas9 molecule having the HNH activity inactivated, e.g., a Cas9 molecule having a mutation at H840, e.g., the H840A.

In an embodiment, the gRNAs are used with a Cas9 nickase molecule having RuvC activity, e.g., a Cas9 molecule having the HNH activity inactivated, e.g., a Cas9 molecule having a mutation at N863, e.g., the N863A.

### VI. Target Cells

Cas9 molecules and gRNA molecules, e.g., a Cas9 molecule/gRNA molecule complex, can be used to manipulate a cell, e.g., to edit a target nucleic acid, in a wide variety of cells.

In an embodiment, a cell is manipulated by altering or editing (e.g., introducing a mutation in) the CCR5 target gene, e.g., as described herein. In an embodiment, the expression of the CCR5target gene is altered or modulated, e.g., in vivo. In another embodiment, the expression of the CCR5 target gene is altered or modulated, e.g., ex vivo.

The Cas9 and gRNA molecules described herein can be delivered to a target cell. In an embodiment, the target cell is a circulating blood cell, e.g., a T cell (e.g., a CD4+ T cell, a CD8+ T cell, a helper T cell, a regulatory T cell, a cytotoxic T cell, a memory T cell, a T cell precursor or a natural killer T cell), a B cell (e.g., a progenitor B cell, a Pre B cell, a Pro B cell, a memory B cell, a plasma B cell), a monocyte, a megakaryocyte, a neutrophil, an eosinophil, a basophil, a mast cell, a reticulocyte, a lymphoid progenitor cell, a myeloid progenitor cell, a gut-associated lymphoid tissue (GALT) cell, a dendritic cell, a macrophage, a microglial cell, or a hematopoietic stem cell. In an embodiment, the target cell is a bone marrow cell, (e.g., a lymphoid progenitor cell, a myeloid progenitor cell, an erythroid progenitor cell, a hematopoietic stem cell, or a mesenchymal stem cell). In an embodiment, the target cell is a CD4+ T cell. In an embodiment, the target cell is a lymphoid progenitor cell (e.g. a common lymphoid progenitor (CLP) cell). In an embodiment, the target cell is a myeloid progenitor cell (e.g. a common myeloid progenitor (CMP) cell). In an embodiment, the target cell is a hematopoietic stem cell (e.g. a long term hematopoietic stem cell (LT-HSC), a short term hematopoietic stem cell (ST-HSC), a multipotent progenitor (MPP) cell, a lineage restricted progenitor (LRP) cell).

In an embodiment, the target cell is manipulated ex vivo by editing (e.g., introducing a mutation in) the CCR5 target gene and/or modulating the expression of the CCR5 target gene, and administered to the subject. Sources of target cells for ex vivo manipulation may include, by way of example, the subject's blood, the subject's cord blood, or the subject's bone marrow. Sources of target cells for ex vivo manipulation may also include, by way of example, heterologous donor blood, cord blood, or bone marrow.

In an embodiment, a CD4+T cell is removed from the subject, manipulated ex vivo as described above, and the CD4+T cell is returned to the subject. In an embodiment, a lymphoid progenitor cell is removed from the subject, manipulated ex vivo as described above, and the lymphoid progenitor cell is returned to the subject. In an embodiment, a myeloid progenitor cell is removed from the subject, manipulated ex vivo as described above, and the myeloid progenitor cell is returned to the subject. In an embodiment, a hematopoietic stem cell is removed from the subject, manipulated ex vivo as described above, and the hematopoietic stem cell is returned to the subject.

A suitable cell can also include a stem cell such as, by way of example, an embryonic stem cell, an induced pluripotent stem cell, a hematopoietic stem cell, a neuronal stem cell and a mesenchymal stem cell. In an embodiment, the cell is an induced pluripotent stem cells (iPS) cell or a cell derived from an iPS cell, e.g., an iPS cell generated from the subject, modified to correct the mutation and differentiated into a clinically relevant cell such as e.g, a CD4+ T cell, a lymphoid progenitor cell, myeloid progenitor cell, a macrophage, dendritic cell, gut associated lymphoid tissue or a hematopoietic stem cell. In an embodiment, AAV is used to transduce the target cells, e.g., the target cells described herein.

### VII. Delivery, Formulations and Routes of Administration

The components, e.g., a Cas9 molecule and gRNA molecule can be delivered or formulated in a variety of forms, see, e.g., Tables 14 and 15. In an embodiment, one Cas9 molecule and two or more (e.g., 2, 3, 4, or more) different gRNA molecules are delivered, e.g., by an AAV vector. In an embodiment, the sequence encoding the Cas9 molecule and the sequence(s) encoding the two or more (e.g., 2, 3, 4, or more) different gRNA molecules are present on the same nucleic acid molecule, e.g., an AAV vector. When a Cas9 or gRNA component is encoded as DNA for delivery, the DNA will typically but not necessarily include a control region, e.g., comprising a promoter, to effect expression. Useful promoters for Cas9 molecule sequences include CMV, EFS, EF-1a, MSCV, PGK, CAG control promoters. In an embodiment, the promoter is a constitutive promoter. In another embodiment, the promoter is a tissue specific promoter. Useful promoters for gRNAs include H1, 7SK, tRNA, and U6 promoters. Promoters with similar or dissimilar strengths can be selected to tune the expression of components. Sequences encoding a Cas9 molecule can comprise a nuclear localization signal (NLS), e.g., an SV40 NLS. In an embodiment, the sequence encoding a Cas9 molecule comprises at least two nuclear localization signals. In an embodiment a promoter for a Cas9 molecule or a gRNA molecule can be, independently, inducible, tissue specific, or cell specific.

Table 14 provides examples of how the components can be formulated, delivered, or administered.

TABLE 14
Elements

| Cas9 | gRNA | |
|---|---|---|
| Mole- | mole- | |
| cule(s) | cule(s) | Comments |
| DNA | DNA | In this embodiment, a Cas9 molecule, typically |
| | | an eaCas9 molecule, and a gRNA are transcribed |
| | | from DNA. In this embodiment, they are |
| | | encoded on separate molecules. |
| DNA | | In this embodiment, a Cas9 molecule, typically |
| | | an eaCas9 molecule, and a gRNA are transcribed |
| | | from DNA, here from a single molecule. |
| DNA | RNA | In this embodiment, a Cas9 molecule, typically |
| | | an eaCas9 molecule, is transcribed from |
| | | DNA, and a gRNA is provided as in vitro |
| | | transcribed or synthesized RNA |
| mRNA | RNA | In this embodiment, a Cas9 molecule, typically |
| | | an eaCas9 molecule, is translated from in vitro |
| | | transcribed mRNA, and a gRNA is provided as |
| | | in vitro transcribed or synthesized RNA. |
| mRNA | DNA | In this embodiment, a Cas9 molecule, typically |
| | | an eaCas9 molecule, is translated from in vitro |
| | | transcribed mRNA, and a gRNA is transcribed |
| | | from DNA. |
| Protein | DNA | In this embodiment, a Cas9 molecule, typically |
| | | an eaCas9 molecule, is provided as a protein, |
| | | and a gRNA is transcribed from DNA. |
| Protein | RNA | In this embodiment, an eaCas9 molecule is |
| | | provided as a protein, and a gRNA is provided |
| | | as transcribed or synthesized RNA. |

Table 15 summarizes various delivery methods for the components of a Cas system, e.g., the Cas9 molecule component and the gRNA molecule component, as described herein.

TABLE 15

| | | Delivery | | | |
|---|---|---|---|---|---|
| | | into Non- | Duration | | Type of |
| | | Dividing | of | Genome | Molecule |
| Delivery Vector/Mode | | Cells | Expression | Integration | Delivered |
| Physical (e.g., | | YES | Transient | NO | Nucleic |
| electroporation, particle gun, | | | | | Acids and |
| Calcium Phosphate | | | | | Proteins |
| transfection, cell compression | | | | | |
| or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic |
| | Liposomes | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated | YES | Transient | NO | Nucleic |
| Non-Viral | Bacteria | | | | Acids |
| Delivery | Engineered | YES | Transient | NO | Nucleic |
| Vehicles | Bacteriophages | | | | Acids |
| | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

DNA-Based Delivery of a Cas9 Molecule and or One or More gRNA Molecule

Nucleic acids encoding Cas9 molecules (e.g., eaCas9 molecules) and/or gRNA molecules, can be administered to subjects or delivered into cells by art-known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof.

DNA encoding Cas9 molecules (e.g., eaCas9 molecules) and/or gRNA molecules can be conjugated to molecules (e.g., N-acetylgalactosamine) promoting uptake by the target cells (e.g., the target cells described herein).

In some embodiments, the Cas9- and/or gRNA-encoding DNA is delivered by a vector (e.g., viral vector/virus or plasmid).

A vector can comprise a sequence that encodes a Cas9 molecule and/or a gRNA molecule. A vector can also comprise a sequence encoding a signal peptide (e.g., for nuclear localization, nucleolar localization, mitochondrial localization), fused, e.g., to a Cas9 molecule sequence. For example, ae vector can comprise a nuclear localization sequence (e.g., from SV40) fused to the sequence encoding the Cas9 molecule.

One or more regulatory/control elements, e.g., a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is recognized by RNA polymerase II (e.g., a CMV promoter). In other embodiments, the promoter is recognized by RNA polymerase III (e.g., a U6 promoter). In some embodiments, the promoter is a regulated promoter (e.g., inducible promoter). In other embodiments, the promoter is a constitutive promoter. In some embodiments, the promoter is a tissue specific promoter. In some embodiments, the promoter is a viral promoter. In other embodiments, the promoter is a non-viral promoter.

In some embodiments, the vector or delivery vehicle is a viral vector (e.g., for generation of recombinant viruses). In some embodiments, the virus is a DNA virus (e.g., dsDNA or ssDNA virus). In other embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in human. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the Cas9 molecule and/or the gRNA molecule. In other embodiments, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In an embodiment, the viral vector recognizes a specific cell type or tissue. For example, the viral vector can be pseudotyped with a different/alternative viral envelope glycoprotein; engineered with a cell type-specific receptor (e.g., genetic modification(s) of one or more viral envelope glycoproteins to incorporate a targeting ligand such as a peptide ligand, a single chain antibody, or a growth factor); and/or engineered to have a molecular bridge with dual specificities with one end recognizing a viral glycoprotein and the other end recognizing a moiety of the target cell surface (e.g., a ligand-receptor, monoclonal antibody, avidin-biotin and chemical conjugation).

Exemplary viral vectors/viruses include, e.g., retroviruses, lentiviruses, adenovirus, adeno-associated virus (AAV), vaccinia viruses, poxviruses, and herpes simplex viruses.

In some embodiments, the Cas9- and/or gRNA-encoding DNA is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the Cas9- and/or gRNA-encoding DNA is delivered by a recombinant lentivirus. For example, the lentivirus is replication-defective, e.g., does not comprise one or more genes required for viral replication.

In some embodiments, the Cas9- and/or gRNA-encoding DNA is delivered by a recombinant adenovirus. In some embodiments, the adenovirus is engineered to have reduced immunity in human.

In some embodiments, the Cas9- and/or gRNA-encoding DNA is delivered by a recombinant AAV. In some embodiments, the AAV does not incorporate its genome into that of a host cell, e.g., a target cell as describe herein. In some embodiments, the AAV can incorporate at least part of its genome into that of a host cell, e.g., a target cell as described herein. In some embodiments, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods. In an embodiment, an AAV capsid that can be used in the methods described herein is a capsid sequence from serotype AAV1, AAV2, AAV3, AAV4, AAV5, AAV6, AAV7, AAV8, AAV9, AAV.rh8, AAV.rh10, AAV.rh32/33, AAV.rh43, AAV.rh64R1, or AAV7m8.

In an embodiment, the Cas9- and/or gRNA-encoding DNA is delivered in a re-engineered AAV capsid, e.g., with 50% or greater, e.g., 60% or greater, 70% or greater, 80% or greater, 90% or greater, or 95% or greater, sequence homology with a capsid sequence from serotypes AAV1, AAV2, AAV3, AAV4, AAV5, AAV6, AAV7, AAV8, AAV9, AAV.rh8, AAV.rh10, AAV.rh32/33, AAV.rh43, or AAV.rh64R1.

In an embodiment, the Cas9- and/or gRNA-encoding DNA is delivered by a chimeric AAV capsid. Exemplary chimeric AAV capsids include, but are not limited to, AAV9i1, AAV2i8, AAV-DJ, AAV2G9, AAV2i8G9, or AAV8G9.

In an embodiment, the AAV is a self-complementary adeno-associated virus (scAAV), e.g., a scAAV that packages both strands which anneal together to form double stranded DNA.

In some embodiments, the Cas9- and/or gRNA-encoding DNA is delivered by a hybrid virus, e.g., a hybrid of one or more of the viruses described herein. In an embodiment, the hybrid virus is hybrid of an AAV (e.g., of any AAV serotype), with a Bocavirus, B19 virus, porcine AAV, goose AAV, feline AAV, canine AAV, or MVM.

A Packaging cell is used to form a virus particle that is capable of infecting a target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, eg. Cas9. For example, an AAV vector used in gene therapy typically only possesses inverted terminal repeat (ITR) sequences from the AAV genome which are required for packaging and gene expression in the host or target cell. The missing viral functions can be supplied in trans by the packaging cell line and/or plasmid containing E2A, E4, and VA genes from adenovirus, and plasmid encoding Rep and Cap genes from AAV, as described in “Triple Transfection Protocol.” Henceforth, the viral DNA is packaged in a cell line, which contains a helper plasmid encoding the other AAV genes, namely rep and cap, but lacking ITR sequences. In embodiment, the viral DNA is packaged in a producer cell line, which contains E1A and/or E1B genes from adenovirus. The cell line is also infected with adenovirus as a helper. The helper virus (e.g., adenovirus or HSV) or helper plasmid promotes replication of the AAV vector and expression of AAV genes from the helper plasmid with ITRs. The helper plasmid is not packaged in significant amounts due to a lack of ITR sequences. Contamination with adenovirus can be reduced by, e.g., heat treatment to which adenovirus is more sensitive than AAV.

In an embodiment, the viral vector has the ability of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In an embodiment, the viral vector achieves cell type specific expression. For example, a tissue-specific promoter can be constructed to restrict expression of the transgene (Cas 9 and gRNA) in only the target cell. The specificity of the vector can also be mediated by microRNA-dependent control of transgene expression. In an embodiment, the viral vector has increased efficiency of fusion of the viral vector and a target cell membrane. For example, a fusion protein such as fusion-competent hemagglutin (HA) can be incorporated to increase viral uptake into cells. In an embodiment, the viral vector has the ability of nuclear localization. For example, a virus that requires the breakdown of the cell wall (during cell division) and therefore will not infect a non-diving cell can be altered to incorporate a nuclear localization peptide in the matrix protein of the virus thereby enabling the transduction of non-proliferating cells.

In some embodiments, the Cas9- and/or gRNA-encoding DNA is delivered by a non-vector based method (e.g., using naked DNA or DNA complexes). For example, the DNA can be delivered, e.g., by organically modified silica or silicate (Ormosil), electroporation, transient cell compression or squeezing (e.g., as described in Lee, et al, 2012, Nano Lett 12: 6322-27), gene gun, sonoporation, magnetofection, lipid-mediated transfection, dendrimers, inorganic nanoparticles, calcium phosphates, or a combination thereof.

In an embodiment, delivery via electroporation comprises mixing the cells with the Cas9- and/or gRNA-encoding DNA in a cartridge, chamber or cuvette and applying one or more electrical impulses of defined duration and amplitude. In an embodiment, delivery via electroporation is performed using a system in which cells are mixed with the Cas9- and/or gRNA-encoding DNA in a vessel connected to a device (e.g, a pump) which feeds the mixture into a cartridge, chamber or cuvette wherein one or more electrical impulses of defined duration and amplitude are applied, after which the cells are delivered to a second vessel.

In some embodiments, the Cas9- and/or gRNA-encoding DNA is delivered by a combination of a vector and a non-vector based method. For example, a virosome comprises a liposome combined with an inactivated virus (e.g., HIV or influenza virus), which can result in more efficient gene transfer, e.g., in a respiratory epithelial cell than either a viral or a liposomal method alone.

In an embodiment, the delivery vehicle is a non-viral vector. In an embodiment, the non-viral vector is an inorganic nanoparticle. Exemplary inorganic nanoparticles include, e.g., magnetic nanoparticles (e.g., Fe₃MnO₂) silica The outer surface of the nanoparticle can be conjugated with a positively charged polymer (e.g., polyethylenimine, polylysine, polyserine) which allows for attachment (e.g., conjugation or entrapment) of payload. In an embodiment, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids for gene transfer are shown below in Table 16.

TABLE 16
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Exemplary polymers for gene transfer are shown below in Table 17.

TABLE 17
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethyleneimine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In an embodiment, the vehicle has targeting modifications to increase target cell update of nanoparticles and liposomes, e.g., cell specific antigens, monoclonal antibodies, single chain antibodies, aptamers, polymers, sugars, and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimuli-cleavable polymer is used, e.g., for release in a cellular compartment. For example, disulfide-based cationic polymers that are cleaved in the reducing cellular environment can be used.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In an embodiment, the vehicle is a genetically modified bacteriophage (e.g., engineered phages having large packaging capacity, less immunogenic, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovescicle (30-100 nm) of endocytic origin (e.g., can be produced from various cell types and can therefore be taken up by cells without the need of for targeting ligands).

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the components of the Cas system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In an embodiment, the nucleic acid molecule is delivered by a different means than one or more of the components of the Cas system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component and/or the gRNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

Delivery of RNA Encoding a Cas9 Molecule

RNA encoding Cas9 molecules (e.g., eaCas9 molecules or eiCas9 molecules) and/or gRNA molecules, can be delivered into cells, e.g., target cells described herein, by art-known methods or as described herein. For example, Cas9-encoding and/or gRNA-encoding RNA can be delivered, e.g., by microinjection, electroporation, transient cell compression or squeezing (e.g., as described in Lee, et al., 2012, Nano Lett 12: 6322-27), lipid-mediated transfection, peptide-mediated delivery, or a combination thereof. Cas9-encoding and/or gRNA-encoding RNA can be conjugated to molecules to promote uptake by the target cells (e.g., target cells described herein).

In an embodiment, delivery via electroporation comprises mixing the cells with the RNA encoding Cas9 molecules (e.g., eaCas9 molecules, eiCas9 molecules or eiCas9 fusion proteins) and/or gRNA molecules in a cartridge, chamber or cuvette and applying one or more electrical impulses of defined duration and amplitude. In an embodiment, delivery via electroporation is performed using a system in which cells are mixed with the RNA encoding Cas9 molecules (e.g., eaCas9 molecules, eiCas9 molecules or eiCas9 fusion proteins) and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

Delivery Cas9 Molecule Protein

Cas9 molecules (e.g., eaCas9 molecules or eiCas9 molecules) can be delivered into cells by art-known methods or as described herein. For example, Cas9 protein molecules can be delivered, e.g., by microinjection, electroporation, transient cell compression or squeezing (e.g., as described in Lee, et al, 2012, Nano Lett 12: 6322-27), lipid-mediated transfection, peptide-mediated delivery, or a combination thereof. Delivery can be accompanied by DNA encoding a gRNA or by a gRNA. Cas9 protein can be conjugated to molecules promoting uptake by the target cells (e.g., target cells described herein).

In an embodiment, delivery via electroporation comprises mixing the cells with the Cas9 molecules (e.g., eaCas9 molecules, eiCas9 molecules or eiCas9 fusion proteins) with or without gRNA molecules in a cartridge, chamber or cuvette and applying one or more electrical impulses of defined duration and amplitude. In an embodiment, delivery via electroporation is performed using a system in which cells are mixed with the Cas9 molecules (e.g., eaCas9 molecules, eiCas9 molecules or eiCas9 fusion proteins) with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

Route of Administration

Systemic modes of administration include oral and parenteral routes. Parenteral routes include, by way of example, intravenous, intrarterial, intraosseous, intramuscular, intradermal, subcutaneous, intranasal and intraperitoneal routes. Components administered systemically may be modified or formulated to target the components to cells of the blood and bone marrow.

Local modes of administration include, by way of example, intra-bone marrow, intrathecal, and intra-cerebroventricular routes. In an embodiment, significantly smaller amounts of the components (compared with systemic approaches) may exert an effect when administered locally (for example, intra-bone marrow) compared to when administered systemically (for example, intravenously). Local modes of administration can reduce or eliminate the incidence of potentially toxic side effects that may occur when therapeutically effective amounts of a component are administered systemically.

In an embodiment, components described herein are delivered by intra-bone marrow injection. Injections may be made directly into the bone marrow compartment of one or more than one bone. In an embodiment, nanoparticle or viral, e.g., AAV vector, delivery is via intra-bone marrow injection.

Administration may be provided as a periodic bolus or as continuous infusion from an internal reservoir or from an external reservoir (for example, from an intravenous bag). Components may be administered locally, for example, by continuous release from a sustained release drug delivery device.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used for intraocular injection. Typically the microspheres are composed of a polymer of lactic acid and glycolic acid, which are structured to form hollow spheres. The spheres can be approximately 15-30 microns in diameter and can be loaded with components described herein.

Bi-Modal or Differential Delivery of Components

Separate delivery of the components of a Cas system, e.g., the Cas9 molecule component and the gRNA molecule component, and more particularly, delivery of the components by differing modes, can enhance performance, e.g., by improving tissue specificity and safety.

In an embodiment, the Cas9 molecule and the gRNA molecule are delivered by different modes, or as sometimes referred to herein as differential modes. Different or differential modes, as used herein, refer modes of delivery that confer different pharmacodynamic or pharmacokinetic properties on the subject component molecule, e.g., a Cas9 molecule or gRNA molecule. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., adeno-associated virus or lentivirus, delivery.

By way of example, the components, e.g., a Cas9 molecule and a gRNA molecule, can be delivered by modes that differ in terms of resulting half-life or persistent of the delivered component the body, or in a particular compartment, tissue or organ. In an embodiment, a gRNA molecule can be delivered by such modes. The Cas9 molecule component can be delivered by a mode which results in less persistence or less exposure to the body or a particular compartment or tissue or organ.

More generally, in an embodiment, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In an embodiment, the first pharmacodynamic or pharmacokinetic property, e.g., distribution, persistence or exposure, is more limited than the second pharmacodynamic or pharmacokinetic property.

In an embodiment, the first mode of delivery is selected to optimize, e.g., minimize, a pharmacodynamic or pharmacokinetic property, e.g., distribution, persistence or exposure.

In an embodiment, the second mode of delivery is selected to optimize, e.g., maximize, a pharmacodynamic or pharmcokinetic property, e.g., distribution, persistence or exposure.

In an embodiment, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In an embodiment, the second mode of delivery comprises a relatively transient element, e.g., an RNA or protein.

In an embodiment, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a Cas9 molecule, is delivered in a transient manner, for example as mRNA or as protein, ensuring that the full Cas9 molecule/gRNA molecule complex is only present and active for a short period of time.

Furthermore, the components can be delivered in different molecular form or with different delivery vectors that complement one another to enhance safety and tissue specificity.

Use of differential delivery modes can enhance performance, safety and efficacy. E.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MEW molecules. A two-part delivery system can alleviate these drawbacks.

Differential delivery modes can be used to deliver components to different, but overlapping target regions. The formation active complex is minimized outside the overlap of the target regions. Thus, in an embodiment, a first component, e.g., a gRNA molecule is delivered by a first delivery mode that results in a first spatial, e.g., tissue, distribution. A second component, e.g., a Cas9 molecule is delivered by a second delivery mode that results in a second spatial, e.g., tissue, distribution. In an embodiment, the first mode comprises a first element selected from a liposome, nanoparticle, e.g., polymeric nanoparticle, and a nucleic acid, e.g., viral vector. The second mode comprises a second element selected from the group. In an embodiment, the first mode of delivery comprises a first targeting element, e.g., a cell specific receptor or an antibody, and the second mode of delivery does not include that element. In embodiment, the second mode of delivery comprises a second targeting element, e.g., a second cell specific receptor or second antibody.

When the Cas9 molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule and the Cas9 molecule are packaged in separated delivery vehicles with distinct but overlapping tissue tropism, the fully functional complex is only be formed in the tissue that is targeted by both vectors.

### Ex Vivo Delivery

In some embodiments, components described in Table 14 are introduced into cells which are then introduced into the subject, e.g., cells are removed from a subject, manipulated ex vivo and then introduced into the subject. Methods of introducing the components can include, e.g., any of the delivery methods described in Table 15.

### VIII. Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein, “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:

(i) alteration, e.g., replacement, of one or both of the non-linking phosphate oxygens and/or of one or more of the linking phosphate oxygens in the phosphodiester backbone linkage;

(ii) alteration, e.g., replacement, of a constituent of the ribose sugar, e.g., of the 2′ hydroxyl on the ribose sugar;

(iii) wholesale replacement of the phosphate moiety with “dephospho” linkers;

(iv) modification or replacement of a naturally occurring nucleobase;

(v) replacement or modification of the ribose-phosphate backbone;

(vi) modification of the 3′ end or 5′ end of the oligonucleotide, e.g., removal, modification or replacement of a terminal phosphate group or conjugation of a moiety; and

(vii) modification of the sugar.

The modifications listed above can be combined to provide modified nucleosides and nucleotides that can have two, three, four, or more modifications. For example, a modified nucleoside or nucleotide can have a modified sugar and a modified nucleobase. In an embodiment, every base of a gRNA is modified, e.g., all bases have a modified phosphate group, e.g., all are phosphorothioate groups. In an embodiment, all, or substantially all, of the phosphate groups of a unimolecular or modular gRNA molecule are replaced with phosphorothioate groups.

In an embodiment, modified nucleotides, e.g., nucleotides having modifications as described herein, can be incorporated into a nucleic acid, e.g., a “modified nucleic acid.” In some embodiments, the modified nucleic acids comprise one, two, three or more modified nucleotides. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in one aspect the modified nucleic acids described herein can contain one or more modified nucleosides or nucleotides, e.g., to introduce stability toward nucleases.

In some embodiments, the modified nucleosides, modified nucleotides, and modified nucleic acids described herein can exhibit a reduced innate immune response when introduced into a population of cells, both in vivo and ex vivo. The term “innate immune response” includes a cellular response to exogenous nucleic acids, including single stranded nucleic acids, generally of viral or bacterial origin, which involves the induction of cytokine expression and release, particularly the interferons, and cell death. In some embodiments, the modified nucleosides, modified nucleotides, and modified nucleic acids described herein can disrupt binding of a major groove interacting partner with the nucleic acid. In some embodiments, the modified nucleosides, modified nucleotides, and modified nucleic acids described herein can exhibit a reduced innate immune response when introduced into a population of cells, both in vivo and ex vivo, and also disrupt binding of a major groove interacting partner with the nucleic acid.

Definitions of Chemical Groups

As used herein, “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 12, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “alkenyl” refers to an aliphatic group containing at least one double bond.

As used herein, “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl.

As used herein, “arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

As used herein, “cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl.

As used herein, “heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl.

As used herein, “heteroaryl” refers to a monovalent radical of a heteroaromatic ring system. Examples of heteroaryl moieties include, but are not limited to, imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

Phosphate Backbone Modifications

The Phosphate Group

In some embodiments, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In some embodiments, the modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

Replacement of the Phosphate Group

The phosphate group can be replaced by non-phosphorus containing connectors. In some embodiments, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Replacement of the Ribophosphate Backbone

Scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include, without limitation, the morpholino, cyclobutyl, pyrrolidine and peptide nucleic acid (PNA) nucleoside surrogates.

Sugar Modifications

The modified nucleosides and modified nucleotides can include one or more modifications to the sugar group. For example, the 2′ hydroxyl group (OH) can be modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative).

“Deoxy” modifications can include hydrogen (i.e. deoxyribose sugars, e.g., at the overhang portions of partially ds RNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the 1′ position on the sugar, e.g., alpha-nucleosides. The modified nucleic acids can also include “abasic” sugars, which lack a nucleobase at C-1′. These abasic sugars can also be further modified at one or more of the constituent sugar atoms. The modified nucleic acids can also include one or more sugars that are in the L form, e.g. L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replaced with α-L-threofuranosyl-(3′→2′)).

Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In some embodiments, the nucleobase can include, for example, naturally-occurring and synthetic derivatives of a base.

Uracil

In some embodiments, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τcm⁵U), 1-taurinomethyl-pseudouridine, 5-taurinomethyl-2-thio-uridine(τm⁵s2U), 1-taurinomethyl-4-thio-pseudouridine, 5-methyl-uridine (m⁵U, i.e., having the nucleobase deoxythymine), 1-methyl-pseudouridine 5-methyl-2-thio-uridine (m⁵s2U), 1-methyl-4-thio-pseudouridine m¹s⁴ψ) 4-thio-1-methyl-pseudouridine, 3-methyl-pseudouridine (m³Ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s2U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s2Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

Cytosine

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m³C), N4-acetyl-cytidine (act), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k²C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

Adenine

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenosine, 7-deaza-8-aza-adenosine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenosine (m²A), N6-methyl-adenosine (m⁶A), 2-methylthio-N6-methyl-adenosine (ms2 m⁶A), N6-isopentenyl-adenosine (i⁶A), 2-methylthio-N6-isopentenyl-adenosine (ms²i⁶A), N6-(cis-hydroxyisopentanyl)adenosine (io⁶A), 2-methylthio-N6-(cis-hydroxyisopentanyl)adenosine (ms2io⁶A), N6-glycinylcarbamoyl-adenosine (g⁶A), N6-threonylcarbamoyl-adenosine (t⁶A), (t⁶A), N6-methyl-N6-threonylcarbamoyl-adenosine 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms2hn⁶A), N6-acetyl-adenosine (ac⁶A), 7-methyl-adenosine, 2-methylthio-adenosine, 2-methoxy-adenosine, α-thio-adenosine, 2′-O-methyl-adenosine (Am), N⁶,2′-O-dimethyl-adenosine (m⁶Am), N⁶-Methyl-2′-deoxyadenosine, N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

Guanine

In some embodiments, the modified nucleobase is a modified guanine. Exemplary nucleobases and nucleosides having a modified guanine include without limitation inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m′G), N2-methyl-guanosine (m²G), N2,N2-dimethyl-guanosine (m²₂G), N2,7-dimethyl-guanosine (m²,7G), N2, N2,7-dimethyl-guanosine (m²,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m′Gm), N2,7-dimethyl-2′-O-methyl-guanosine (m²,7Gm), 2′-O-methyl-inosine (Im), 1,2′-O-dimethyl-inosine (m′Im), O⁶-phenyl-2′-deoxyinosine, 2′-O-ribosylguanosine (phosphate) (Gr(p)), 1-thio-guanosine, O⁶-methyl-guanosine, O⁶-Methyl-2′-deoxyguanosine, 2′-F-ara-guanosine, and 2′-F-guanosine.

Exemplary Modified gRNAs

In some embodiments, the modified nucleic acids can be modified gRNAs. It is to be understood that any of the gRNAs described herein can be modified in accordance with this section, including any gRNA that comprises a targeting domain from Tables 1A-1F, 2A-2C, 3A-3E, 4A-4C, 5A-5C, 6A-6E, 7A-7C, or 18.

As discussed above, transiently expressed or delivered nucleic acids can be prone to degradation by, e.g., cellular nucleases. Accordingly, in one aspect the modified gRNAs described herein can contain one or more modified nucleosides or nucleotides which introduce stability toward nucleases. While not wishing to be bound by theory it is also believed that certain modified gRNAs described herein can exhibit a reduced innate immune response when introduced into a population of cells, particularly the cells of the present invention. As noted above, the term “innate immune response” includes a cellular response to exogenous nucleic acids, including single stranded nucleic acids, generally of viral or bacterial origin, which involves the induction of cytokine expression and release, particularly the interferons, and cell death.

While some of the exemplary modification discussed in this section may be included at any position within the gRNA sequence, in some embodiments, a gRNA comprises a modification at or near its 5′ end (e.g., within 1-10, 1-5, or 1-2 nucleotides of its 5′ end). In some embodiments, a gRNA comprises a modification at or near its 3′ end (e.g., within 1-10, 1-5, or 1-2 nucleotides of its 3′ end). In some embodiments, a gRNA comprises both a modification at or near its 5′ end and a modification at or near its 3′ end.

In an embodiment, the 5′ end of a gRNA is modified by the inclusion of a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)). The cap or cap analog can be included during either chemical synthesis or in vitro transcription of the gRNA.

In an embodiment, an in vitro transcribed gRNA is modified by treatment with a phosphatase (e.g., calf intestinal alkaline phosphatase) to remove the 5′ triphosphate group.

In an embodiment, the 3′ end of a gRNA is modified by the addition of one or more (e.g., 25-200) adenine (A) residues. The polyA tract can be contained in the nucleic acid (e.g., plasmid, PCR product, viral genome) encoding the gRNA, or can be added to the gRNA during chemical synthesis, or following in vitro transcription using a polyadenosine polymerase (e.g., E. coli Poly(A)Polymerase).

In an embodiment, in vitro transcribed gRNA contains both a 5′ cap structure or cap analog and a 3′ polyA tract. In an embodiment, an in vitro transcribed gRNA is modified by treatment with a phosphatase (e.g., calf intestinal alkaline phosphatase) to remove the 5′ triphosphate group and comprises a 3′ polyA tract.

In some embodiments, gRNAs can be modified at a 3′ terminal U ribose. For example, the two terminal hydroxyl groups of the U ribose can be oxidized to aldehyde groups and a concomitant opening of the ribose ring to afford a modified nucleoside as shown below:

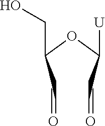

wherein “U” can be an unmodified or modified uridine.

In another embodiment, the 3′ terminal U can be modified with a 2′3′ cyclic phosphate as shown below:

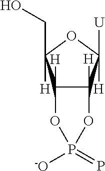

wherein “U” can be an unmodified or modified uridine.

In some embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In some embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In some embodiments, a gRNA can include “locked” nucleic acids (LNA) in which the 2′ OH-group can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In some embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNA molecules include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In an embodiment, a gRNA comprises a 4′-S, 4′-Se or a 4′-C-aminomethyl-2′-O-Me modification.

In some embodiments, deaza nucleotides, e.g., 7-deaza-adenosine, can be incorporated into the gRNA. In some embodiments, 0- and N-alkylated nucleotides, e.g., N6-methyl adenosine, can be incorporated into the gRNA. In some embodiments, one or more or all of the nucleotides in a gRNA molecule are deoxynucleotides.

miRNA Binding Sites

microRNAs (or miRNAs) are naturally occurring cellular 19-25 nucleotide long noncoding RNAs. They bind to nucleic acid molecules having an appropriate miRNA binding site, e.g., in the 3′ UTR of an mRNA, and down-regulate gene expression. While not wishing to be bound by theory it is believed that the down regulation is either by reducing nucleic acid molecule stability or by inhibiting translation. An RNA species disclosed herein, e.g., an mRNA encoding Cas9 can comprise an miRNA binding site, e.g., in its 3′UTR. The miRNA binding site can be selected to promote down regulation of expression is a selected cell type. By way of example, the incorporation of a binding site for miR-122, a microRNA abundant in liver, can inhibit the expression of the gene of interest in the liver.

### EXAMPLES

The following Examples are merely illustrative and are not intended to limit the scope or content of the invention in any way.

### Example 1

### Evaluation of Candidate Guide RNAs (gRNAs)

The suitability of candidate gRNAs can be evaluated as described in this example. Although described for a chimeric gRNA, the approach can also be used to evaluate modular gRNAs.

Cloning gRNAs into Vectors

For each gRNA, a pair of overlapping oligonucleotides is designed and obtained. Oligonucleotides are annealed and ligated into a digested vector backbone containing an upstream U6 promoter and the remaining sequence of a long chimeric gRNA. Plasmid is sequence-verified and prepped to generate sufficient amounts of transfection-quality DNA. Alternate promoters maybe used to drive in vivo transcription (e.g. H1 promoter) or for in vitro transcription (e.g., a T7 promoter).

Cloning gRNAs in Linear dsDNA Molecule (STITCHR)

For each gRNA, a single oligonucleotide is designed and obtained. The U6 promoter and the gRNA scaffold (e.g. including everything except the targeting domain, e.g., including sequences derived from the crRNA and tracrRNA, e.g., including a first complementarity domain; a linking domain; a second complementarity domain; a proximal domain; and a tail domain) are separately PCR amplified and purified as dsDNA molecules. The gRNA-specific oligonucleotide is used in a PCR reaction to stitch together the U6 and the gRNA scaffold, linked by the targeting domain specified in the oligonucleotide. Resulting dsDNA molecule (STITCHR product) is purified for transfection. Alternate promoters may be used to drive in vivo transcription (e.g., H1 promoter) or for in vitro transcription (e.g., T7 promoter). Any gRNA scaffold may be used to create gRNAs compatible with Cas9s from any bacterial species.

Initial gRNA Screen

Each gRNA to be tested is transfected, along with a plasmid expressing Cas9 and a small amount of a GFP-expressing plasmid into human cells. In preliminary experiments, these cells can be immortalized human cell lines such as 293T, K562 or U2OS. Alternatively, primary human cells may be used. In this case, cells may be relevant to the eventual therapeutic cell target (e.g., a circulating blood cell, e.g., a T cell (e.g., a CD4+ T cell, a CD8+ T cell, a helper T cell, a regulatory T cell, a cytotoxic T cell, a memory T cell, a T cell precursor or a natural killer T cell)). The use of primary cells similar to the potential therapeutic target cell population may provide important information on gene targeting rates in the context of endogenous chromatin and gene expression.

Transfection may be performed using lipid transfection (such as Lipofectamine or Fugene) or by electroporation (such as Lonza Nucleofection). Following transfection, GFP expression can be determined either by fluorescence microscopy or by flow cytometry to confirm consistent and high levels of transfection. These preliminary transfections can comprise different gRNAs and different targeting approaches (17-mers, 20-mers, nuclease, dual-nickase, etc.) to determine which gRNAs/combinations of gRNAs give the greatest activity.

Efficiency of cleavage with each gRNA may be assessed by measuring NHEJ-induced indel formation at the target locus by a T7E1-type assay or by sequencing. Alternatively, other mismatch-sensitive enzymes, such as Cell/Surveyor nuclease, may also be used.

For the T7E1 assay, PCR amplicons are approximately 500-700 bp with the intended cut site placed asymmetrically in the amplicon. Following amplification, purification and size-verification of PCR products, DNA is denatured and re-hybridized by heating to 95° C. and then slowly cooling. Hybridized PCR products are then digested with T7 Endonuclease I (or other mismatch-sensitive enzyme) which recognizes and cleaves non-perfectly matched DNA. If indels are present in the original template DNA, when the amplicons are denatured and re-annealed, this results in the hybridization of DNA strands harboring different indels and therefore lead to double-stranded DNA that is not perfectly matched. Digestion products may be visualized by gel electrophoresis or by capillary electrophoresis. The fraction of DNA that is cleaved (density of cleavage products divided by the density of cleaved and uncleaved) may be used to estimate a percent NHEJ using the following equation: % NHEJ=(1−(1−fraction cleaved)^{1/2}). The T7E1 assay is sensitive down to about 2-5% NHEJ.

Sequencing may be used instead of, or in addition to, the T7E1 assay. For Sanger sequencing, purified PCR amplicons are cloned into a plasmid backbone, transformed, miniprepped and sequenced with a single primer. Sanger sequencing may be used for determining the exact nature of indels after determining the NHEJ rate by T7E1.

Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). This method allows for detection of very low NHEJ rates.

### Example 2

### Assessment of Gene Targeting by NHEJ

The gRNAs that induce the greatest levels of NHEJ in initial tests can be selected for further evaluation of gene targeting efficiency. In this case, cells are derived from disease subjects and, therefore, harbor the relevant mutation.

Following transfection (usually 2-3 days post-transfection) genomic DNA may be isolated from a bulk population of transfected cells and PCR may be used to amplify the target region. Following PCR, gene targeting efficiency to generate the desired mutations (either knockout of a target gene or removal of a target sequence motif) may be determined by sequencing. For Sanger sequencing, PCR amplicons may be 500-700 bp long. For next generation sequencing, PCR amplicons may be 300-500 bp long. If the goal is to knockout gene function, sequencing may be used to assess what percent of alleles have undergone NHEJ-induced indels that result in a frameshift or large deletion or insertion that would be expected to destroy gene function. If the goal is to remove a specific sequence motif, sequencing may be used to assess what percent of alleles have undergone NHEJ-induced deletions that span this sequence.

### Example 3

### Screening of gRNAs for CCR5

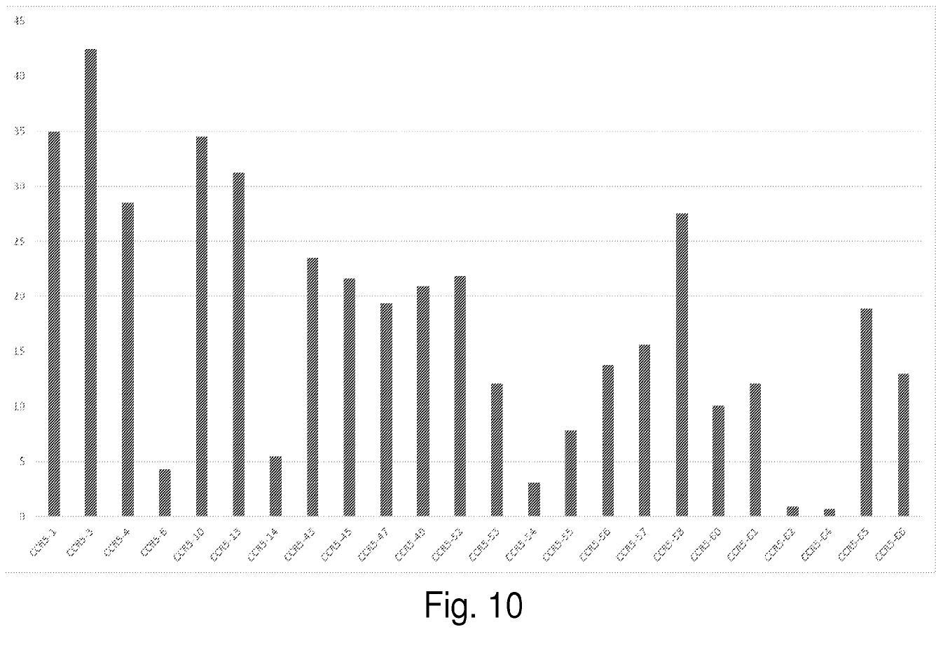

In order to identify gRNAs with the highest on target NHEJ efficiency, 24 S. pyogenes gRNAs were selected for testing (Table 18). A DNA plasmid comprised of an exemplary gRNA (including the target region and appropriate TRACR sequence) under the control of a U6 promoter was generated by restriction enzyme cloning. This DNA template was subsequently transfected into 293 cells using Lipofectamine 3000 along with a DNA plasmid encoding the appropriate Cas9 downstream of a CMV promoter. Genomic DNA was isolated from the cells 48-72 hours post transfection. To determine the rate of modification at the CCR5 gene, the target region was amplified using a locus PCR with the following primers (CCR5 exon 3 5′ primer: TATCAAGTGTCAAGTCCAATCTATGACATC (SEQ ID NO: 5752); CCR5 exon 3 3′ primer: GGAAATTCTTCCAGAATTGATACTGACTG (SEQ ID NO: 5753). After PCR amplification, a T7E1 assay was performed on the PCR product. Briefly, this assay involves melting the PCR product followed by a re-annealing step. If gene modification has occurred, there will exist double stranded products that are not perfect matches due to some frequency of insertions or deletions. These double stranded products are sensitive to cleavage by a T7 endonuclease 1 enzyme at the site of mismatch. Therefore, the efficiency of cutting by the Cas9/gRNA complex can be determined by analyzing the amount of T7E1 cleavage. The formula that is used to provide a measure of % NHEJ from the T7E1 cutting is the following: 100*(1−((1−(fraction cleaved))̂0.5)). The results of this analysis are shown in FIG. 10.

TABLE 18

| gRNA | Targeting Domain Sequence | SEQ ID NO |
|---|---|---|
| CCR5-1 | GCCUCCGCUCUACUCAC | 396 |
| CCR5-3 | GCCGCCCAGUGGGACUU | 397 |
| CCR5-4 | GCAUAGUGAGCCCAGAA | 401 |
| CCR5-6 | GCCUUUUGCAGUUUAUC | 409 |
| CCR5-10 | GACAAUCGAUAGGUACC | 399 |
| CCR5-13 | GACAAGUGUGAUCACUU | 404 |
| CCR5-14 | GGUACCUAUCGAUUGUC | 402 |
| CCR5-43 | GCUGCCGCCCAGUGGGACUU | 388 |
| CCR5-45 | GGUACCUAUCGAUUGUCAGG | 394 |
| CCR5-47 | GCAGCAUAGUGAGCCCAGAA | 393 |
| CCR5-49 | GUGAGUAGAGCGGAGGCAGG | 395 |
| CCR5-52 | AUGUGUCAACUCUUGAC | 398 |
| CCR5-53 | UUGACAGGGCUCUAUUUUAU | 499 |
| CCR5-54 | ACAGGGCUCUAUUUUAU | 5749 |
| CCR5-55 | UCAUCCUCCUGACAAUCGAU | 477 |
| CCR5-56 | UCCUCCUGACAAUCGAU | 5750 |
| CCR5-57 | CCUGACAAUCGAUAGGUACC | 463 |
| CCR5-58 | GGUGACAAGUGUGAUCACUU | 4469 |
| CCR5-60 | CCAGGUACCUAUCGAUUGUC | 391 |
| CCR5-61 | ACCUAUCGAUUGUCAGG | 5751 |
| CCR5-62 | UCAGCCUUUUGCAGUUUAUC | 476 |
| CCR5-64 | CACAUUGAUUUUUUGGC | 400 |
| CCR5-65 | AGUAGAGCGGAGGCAGG | 442 |
| CCR5-66 | CCUGCCUCCGCUCUACUCAC | 387 |

### Example 4

### Assessment of Gene Targeting in Hematopoietic Stem Cells

Transplantation of autologous CD34⁺ hematopoietic stem cells (HSCs) that have been genetically modified to prevent expression of the wild-type CCR5 gene product prevents entry of the HIV virus HSC progeny that are normally susceptible to HIV infection (e.g., macrophages and CD4 T-lymphocytes). Clinically, transplantation of HSCs that contain a genetic mutation in the coding sequence for the CCR5 chemokine receptor has been shown to control HIV infection long-term (Witter et. al, New England Journal of Medicine, 2009; 360(7):692-698). Genome editing with the CRISPR/Cas9 platform precisely alters endogenous gene targets by creating an indel at the targeted cut site that can lead to knock down of gene expression at the edited locus. In this Example, genome editing in human mobilized peripheral blood CD34⁺ HSCs after co-delivery of Cas9 with gRNA targeting the CCR5 locus was evaluated to induce gene editing in CD34⁺ cells.

Human CD34⁺ HSCs cells from mobilized peripheral blood (AllCells) were thawed into StemSpan Serum-Free Expansion Medium (SFEM™, StemCell Technologies) containing 100 ng/mL each of the following cytokines: human stem cell factor (SCF), thrombopoietin (TPO), and flt-3 ligand (FL) (all from Peprotech). Cells were grown for 3 days in a humidified incubator and 5% CO₂ 20% O₂. On day 3, media was replaced with fresh Stemspan-SFEM™ supplemented with human SCF, TPO, FL and 40 nM of the small molecule UM171 (Xcess Bio), a human HSC self-renewal agonist which has been shown to support robust expansion of human HSCs (Fares et. al, Science, 2014; 345(6203):1509-1512). The published use of UM171 involved prolonged exposure of HSCs to the small molecule for ex vivo expansion of HSCs. In the current experiment, HSCs were exposed to UM171 for 2 hours before and 24 hours after delivery of Cas9 and gRNA plasmid DNA. This UM171 treatment protocol was based on the pilot studies that indicated acute pre-treatment with UM171 before lentivirus vector mediated gene delivery improved HSC viability compared to HSCs treated with vehicle (dimethylsulfoxide, DMSO, Sigma) alone. After the 2-hour pretreatment with UM171, 1 million CD34⁺ HSCs were Nucleofected™ with the Amaxa™ 4D Nucleofector™ device (Lonza), Program EO100 using components of the P3 Primary Cell 4D-Nucleofector Kit™ (Lonza) according to the manufacturer's instructions. Briefly, one million cells were suspended in Nucleofector™ solution and the following amounts of plasmid DNA were added to the cell suspension: 1250 ng plasmid expressing CCR5 gRNA (CCR5-43) from the human U6 promoter and 3750 ng plasmid expressing wild-type S. pyogenes Cas 9 transcriptionally regulated by the CMV promoter. After Nucleofection™, cells were plated into Stemspan-SFEM™ supplemented with SCF, TPO, FL and 40 nM UM171. After overnight incubation, HSCs were plated in Stemspan-SFEM™ plus cytokines without UM171. At 96 hours after Nucleofection™, CD34⁺ cells were counted for by trypan blue exclusion and divided into 3 portions for the following analyses: a) flow cytometry analysis for assessment of viability by co-staining with 7-Aminoactinomycin-D (7-AAD) and allophycocyanin (APC)-conjugated Annexin-V antibody (ebioscience); b) flow cytometry analysis for maintenance of HSC phenotype (after co-staining with phycoerythrin (PE)-conjugated anti-human CD34 antibody and fluorescein isothicyanate (FITC)-conjugated anti-human CD90, both from BD Bioscience; c) hematopoietic colony forming cell (CFC) analysis by plating 1500 cells in semi-solid methylcellulose based Methocult medium (StemCell Technologies) that supports differentiation of erythroid and myeloid blood cell colonies from HSCs and serves as a surrogate assay to evaluate HSC multipotency and differentiation potential ex vivo; d) genomic DNA analysis for detection of editing at the CCR5 locus. Genomic DNA was extracted from HSCs 96 hours after Nucleofection™, and CCR5 locus-specific PCR reactions were performed.

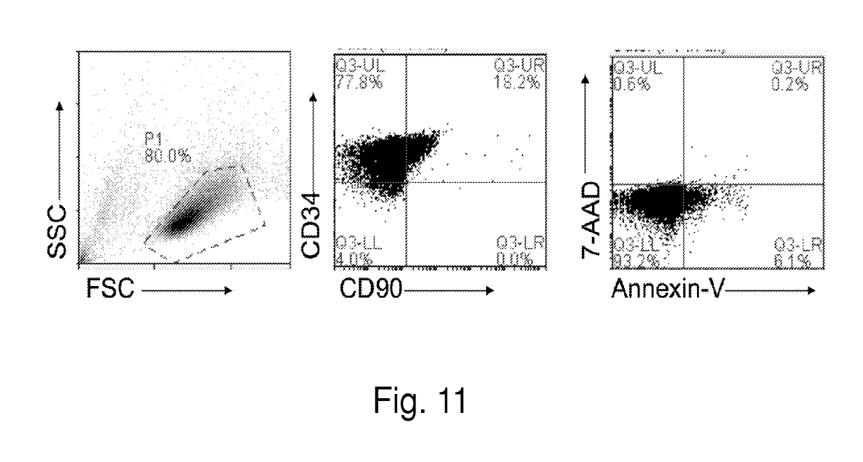

HSCs that were Nucleofected™ with Cas9 and CCR5 gRNA plasmids after pre-treatment with UM171 exhibited >93% viability (7-AAD⁻ AnnexinV⁻) and maintained co-expression of CD34 and CD90, as determined by flow cytometry analysis (FIG. 11). In addition, the UM171-treated Nucleofected™ cells were able to divide, as there was an increase in cell number with a fold-expansion similar to the level achieved win unelectroporated HSCS (Table 19). In contrast, HSCs Nucleofected™ without UM171 pre-treatment had decreased viability and cell did not expand in culture.

Table 19 shows that UM171 preserved CD34+ HSC viability after Nucleofection™ with wild type Cas9 and CCR5-43 gRNA plasmid DNA (96 hours)

TABLE 19

| | Fold expansion of |
|---|---|
| Condition | CD34⁺ cells (96 hours) |
| No Nucleofection ™ | 1.6 |
| Nucleofection ™ + UM171 treatment | 1.5 |
| Nucleofection ™ + vehicle treatment | 0.6 |

In order to detect indels at the CCR5 locus, T7E1 assays were performed on CCR5 locus-specific PCR products that were amplified from genomic DNA samples from Nucleofected™ CD34⁺ HSCs and then percentage of indels detected at the CCR5 locus was calculated. Twenty percent indels was detected in the genomic DNA from CD34⁺ HSCs Nucleofected™ with Cas9 and CCR5 gRNA plasmids after pre-treatment with UM171.

To evaluate maintenance of HSC potency and differentiation potential, two weeks after plating CD34⁺ HSCs in CFC assays, hematopoietic activity was quantified based on scoring the HSC progeny by enumerating the total number of hematopoietic colony forming units (CFU) and the frequencies of specific blood cell phenotypes, including: mixed myeloid/erythroid (Granulocyte-erythroid-monocyte macrophage, CFU-GEMM), myeloid (CFU-macrophage (M), granulocyte-macrophage (CFU-GM)) and erythroid (CFU-E) colonies. CD34⁺ HSCs that were Nucleofected™ after UM171 pre-treatment maintained CFC potential compared to un-Nucleofected™ HSCs (Table 20). In contrast, CD34⁺ HSCs that were Nucleofected™ without UM171 pre-treatment had reduced CFC potential (lower total CFC counts and reduced numbers of mixed-phenotype colonies (CFU-GEMM) and erythroid colonies (CFU-E)) in comparison to un-Nucleofected™ CD34⁺ HSCs.

Table 20 shows that UM171 preserved CD34+ HSC viability after Nucleofection™ with wild-type Cas9 and CCR5-43 gRNA plasmid DNA (two weeks).

TABLE 20

| | Number of colony forming units per 1500 | | | | | |
|---|---|---|---|---|---|---|
| | CD34⁺ HSCs plated | | | | | |
| Condition | E | G | M | GM | GEMM | Total |
| No Nucleofection ™ | 64 | 3 | 88 | 5 | 11 | 171 |
| Nucleofection ™ + UM171 | 92 | 40 | 64 | 32 | 20 | 228 |
| Nucleofection ™ + vehicle | 18 | 22 | 6 | 1 | 1 | 28 |

Delivery of co-delivery wild-type S. pyogenes Cas9 and a single CCR5 gRNA plasmid DNA supported 20% genome editing of CD34⁺ HSCs, without loss of cell viability, multipotency, self-renewal and differentiation potential. Pre-treatment and short-term (24-hour) co-culture with the HSC self-renewal agonist UM171 was critical for maintenance of HSC survival and proliferation after Nucleofection™ with Cas9/gRNA DNA. Clinically, transplantation of HSCs that contain a genetic mutation in the CCR5 gene generated by CRISPR/Cas9 related methods can be used to achieve long term control of HIV infection.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

Other embodiments are within the following claims.

What is claimed is:

1. A CRISPR/Cas system, comprising:
a gRNA molecule comprising a targeting domain which is complementary with a target sequence of a C-C chemokine receptor type 5 (CCR5) gene; and
a Cas9 molecule.

2. The system of claim 1, wherein said system is configured to forma double strand break or a single strand break within 500 bp, 450 bp, 400 bp, 350 bp, 300 bp, 250 bp, 200 bp, 150 bp, 100 bp, 50 bp, 25 bp, or 10 bp of a CCR5 target position, thereby altering said CCR5 gene.

3. The system of claim 2, wherein said CCR5 target position is selected from the group consisting of CCR5 target knockout positions, CCR5 target knockdown positions, CCR5 target point positions, and CCR5 target hotspot mutations.

4. The system of claim 1, wherein said Cas9 molecule is selected from the group consisting of an enzymatically active Cas9 (eaCas9) molecule, an enzymatically inactive Cas9 (eiCas9) molecule, and an eiCas9 fusion protein.

5. The system of claim 4, wherein said eaCas9 molecule comprises HNH-like domain cleavage activity but has no, or no significant, N-terminal RuvC-like domain cleavage activity.

6. The system of claim 4, wherein said eaCas9 molecule is an HNH-like domain nickase.

7. The system of claim 4, wherein said eaCas9 molecule comprises a mutation at D10.

8. The system of claim 4, wherein said eaCas9 molecule comprises N-terminal RuvC-like domain cleavage activity but has no, or no significant, HNH-like domain cleavage activity.

9. The system of claim 4, wherein said eaCas9 molecule is an N-terminal RuvC-like domain nickase.

10. The system of claim 4, wherein said eaCas9 molecule comprises a mutation at H840 or N863.

11. The system of claim 4, wherein said eiCas9 fusion protein is an eiCas9-transcription repressor domain fusion.

12. The system of claim 1, wherein said Cas9 molecule is an S. aureus Cas9 molecule, an S. pyogenes Cas9 molecule, or a N. meningitidis Cas9 molecule.

13. The system of claim 2, wherein said altering said CCR5 gene comprises knocking out said CCR5 gene, or knocking down said CCR5 gene.

14. The system of claim 1, wherein said targeting domain is configured to target a coding region or a non-coding region of said CCR5 gene, wherein said non-coding region comprises a promoter region, an enhancer region, an intron, the 3′ UTR, the 5′ UTR, or a polyadenylation signal region of said CCR5 gene; and said coding region comprises an exon of said CCR5 gene.

15. The system of claim 1, wherein said targeting domain comprises or consists of a nucleotide sequence that is the same as, or differs by no more than 3 nucleotides from, a targeting domain sequence selected from the targeting domain sequences disclosed in Tables 1A-1F, 2A-2C, 3A-3E, 4A-4C, 5A-5C, 6A-6E, 7A-7C, and 18.

16. The system of claim 1, wherein said gRNA is a modular gRNA molecule or a chimeric gRNA molecule.

17. The system of claim 1, wherein said targeting domain has a length of 16, 17, 18, 19, 20, 21, 22, 23, 24, 25, or 26 nucleotides.

18. The system of claim 1, wherein said gRNA molecule comprises from 5′ to 3′:
a targeting domain;
a first complementarity domain;
a linking domain;
a second complementarity domain;
a proximal domain; and
a tail domain.

19. The system of claim 18, wherein said linking domain is no more than 25 nucleotides in length.

20. The system of claim 18, wherein said proximal and tail domain, taken together, are at least 20, at least 25, at least 30, or at least 40 nucleotides in length.

21. A cell transfected with the CRISPR/Cas system of claim 1.

22. A gRNA molecule comprising a targeting domain which is complementary with a target sequence of a CCR5 gene.

23. The gRNA molecule of claim 22, wherein said targeting domain comprises or consists of a nucleotide sequence that is the same as, or differs by no more than 3 nucleotides from, a targeting domain sequence selected from the targeting domain sequences disclosed in Tables 1A-1F, 2A-2C, 3A-3E, 4A-4C, 5A-5C, 6A-6E, 7A-7C, and 18.

24. A composition comprising the gRNA molecule of claim 22.

25. The composition of claim 24, further comprising a Cas9 molecule.

26. A nucleic acid composition that comprises: (a) a first nucleotide sequence that encodes a gRNA molecule comprising a targeting domain that is complementary with a target sequence of a CCR5 gene.

27. The nucleic acid composition of claim 26, further comprising: (b) a second nucleotide sequence that encodes a Cas9 molecule.

28. The nucleic acid of claim 27, wherein said Cas9 molecule is selected from the group consisting of an eaCas9 molecule, an eiCas9 molecule, and an eiCas9 fusion protein.

29. The nucleic acid of claim 27, wherein said Cas9 molecule is an S. aureus Cas9 molecule, an S. pyogenes Cas9 molecule, or a N. meningitidis Cas9 molecule.

30. The nucleic acid composition of claim 27, wherein (a) and (b) are present on one nucleic acid molecule; or (a) is present on a first nucleic acid molecule and (b) is present on a second nucleic acid molecule.

31. The nucleic acid composition of claim 30, wherein each of said nucleic acid molecule, said first nucleic acid molecule, and said second nucleic acid molecule is a DNA plasmid.

32. The nucleic acid composition of claim 26, further comprising: (c) a third nucleotide sequence that encodes a second gRNA molecule comprising a targeting domain that is complementary with a second target sequence of said CCR5 gene.

33. A cell transfected with the nucleic acid composition of claim 26.

34. A method of altering a CCR5 gene in a cell, comprising administering to said cell:
(i) a CRISPR/Cas system comprising: (a) a gRNA molecule comprising a targeting domain which is complementary with a target domain sequence of said CCR5 gene and (b) a Cas9 molecule; or
(ii) a nucleic acid composition that comprises: (a) a first nucleotide sequence encoding a gRNA molecule comprising a targeting domain that is complementary with a target sequence of a CCR5 gene and (b) a second nucleotide sequence encoding a Cas9 molecule.

35. The method of claim 34, wherein said alteration comprises knockout of said CCR5 gene or knockdown of said CCR5 gene.

36. The method of claim 35, wherein said knockout of said CCR5 gene comprises:
(a) insertion or deletion of one or more nucleotides in close proximity to or within the early coding region of said CCR5 gene, or
(b) deletion of a genomic sequence comprising at least a portion of said CCR5 gene.

37. The method of claim 35, wherein said alteration comprises knockdown of said CCR5 gene and said Cas9 molecule is an eiCas9 molecule or an eiCas9 fusion protein.

38. The method of claim 34, wherein said alteration of said CCR5 gene results in reduction or elimination of (a) expression of said CCR5 gene, (b) CCR5 protein function, and/or (c) level of CCR5 protein.

39. The method of claim 34, wherein said cell is from a subject suffering from or at risk for HIV infection or AIDS.

40. The method of claim 34, wherein said cell is selected from the group consisting of a stem cell, a progenitor cell, a T cell, a B cell, and a blood cell.

41. The method of claim 34, wherein said cell is a hematopoietic stem cell.
